(19) United States
(12) Patent Application Publication
(10) Pub. No.: US 2024/0410018 A1
(43) Pub. Date: Dec. 12, 2024

(54) NOVEL KINASE FUSIONS DETECTED BY LIQUID BIOPSY

(71) Applicant: FOUNDATION MEDICINE, INC., Boston, MA (US)

(72) Inventors: Jessica K. LEE, Somerville, MA (US); Alexa B. SCHROCK, Okemos, MI (US); Mehlika HAZAR-RETHINAM, Brisbane (AU); Geoffrey R. OXNARD, Arlington, MA (US)

(73) Assignee: Foundation Medicine, Inc., Boston, MA (US)

(21) Appl. No.: 18/702,751

(22) PCT Filed: Oct. 28, 2022
(86) PCT No.: PCT/US2022/078934

§ 371 Date: Apr. 18, 2024

Related U.S. Application Data
(60) Provisional application No. 63/273,794, filed on Oct. 29, 2021.

(51) Int. Cl. C12Q 1/6886

(52) U.S. Cl. CPC C12Q 1/6886; C12Q 2600/156; G01N 2800/52; G01N 2800/54

(57) ABSTRACT

Provided herein are kinase fusion nucleic acid molecules and polypeptides, methods related to detecting kinase fusion nucleic acid molecules and polypeptides in cancer, as well as methods of treatment and uses related thereto. Detection of a kinase fusion nucleic acid molecule or polypeptide can be used to identify individuals that may benefit from treatment with an anti-cancer therapy.

### CROSS-REFERENCE TO RELATED APPLICATIONS

This application claims the benefit of U.S. Provisional Application No. 63/273,794, filed Oct. 29, 2021, which is hereby incorporated by reference in its entirety.

### REFERENCE TO AN ELECTRONIC SEQUENCE LISTING

The contents of the electronic sequence listing (197102007640seglist.xml; Size: 78,680 bytes; and Date of Creation: Oct. 26, 2022) are herein incorporated by reference in their entirety.

### TECHNICAL FIELD

Provided herein are kinase fusion nucleic acid molecules and polypeptides, methods related to detecting such kinase fusion nucleic acid molecules and polypeptides, as well as methods of diagnosis/treatment and uses related thereto.

### BACKGROUND

Kinases activated by gene fusions are established oncogenic drivers and therapeutic targets, and have been associated with both hematopoietic malignancies and solid tumors. For example, a number of tyrosine kinase gene fusions (e.g., of the NTRK family) have been identified across several cancers. Recently, approvals of NTRK inhibitors have led to routine diagnostic testing for NTRK fusions across many cancer types (Cocco et al. (2018) Nat Rev Clin Oncol, 15:731-747).

Kinase fusions have also been observed in patients following initial treatment with targeted therapies, suggesting that kinase fusions may be an acquired resistance (AR) mechanism, and that patients with such fusions could benefit from strategies that target the acquired kinase fusion. See, e.g, Xu et al., Cancer Manag Res (2019) 11:6343-51; Piotrowska et al., Cancer Discov (2018) 8(12):1529-39; Schrock et al., J Thorac Oncol (2018) 13(9):1312-23; and Schrock et al., J Thorac Oncol 2019; 14(2):255-64).

Liquid biopsies for genomic profiling have the advantage of being less invasive than traditional tissue biopsies, while potentially generating insights into tumor heterogeneity (Bettegowda et al. (2014) Sci Transl Med, 6:224ra24; Gerlinger et al. (2012) N Engl J Med, 366:883-892; Piotrowska et al. (2015) Cancer Discov, 5:713-722; Diaz et al. (2012) Nature, 486:537-540; Kwak et al. (2015) Cancer Discov, 5:1271-1281; and Russo et al. (2016) Cancer Discov, 6:147-153). However, kinase fusions can be challenging to detect in liquid biopsies, e.g., in circulating tumor (ctDNA), and tissue-liquid concordance varies widely (see, e.g., Paweletz et al. (2016) Clin Cancer Res, 22:915-922; Muller et al. (2017) J Thorac Oncol, 12:1503-1511; Supplee et al. (2019) Lung Cancer, 134:96-99; and Gupta et al. (2020) Oncologist, 25: 235-243).

Thus, there is a need in the art for characterizing the pan-cancer landscape of kinase fusions, and for developing methods, compositions, and assays for evaluating and treating patients with such fusions, e.g., detected through liquid biopsies (e.g., in ctDNA) and/or tissue biopsies.

All references cited herein, including patents, patent applications and publications, are hereby incorporated by reference in their entirety. To the extent that any reference incorporated by reference conflicts with the instant disclosure, the instant disclosure shall control.

### SUMMARY OF THE INVENTION

In some aspects, provided herein is a method of identifying an individual having a cancer who may benefit from a treatment comprising an anti-cancer therapy, the method comprising detecting in a sample from the individual a fusion nucleic acid molecule, or a fusion polypeptide encoded by the fusion nucleic acid molecule, wherein: (a) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or (b) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2; wherein detection of the fusion nucleic acid molecule or fusion polypeptide encoded by the fusion nucleic acid molecule in the sample identifies the individual as one who may benefit from the treatment comprising the anti-cancer therapy.

In another aspect, provided herein is a method of selecting a treatment for an individual having a cancer, the method comprising detecting in a sample from the individual a fusion nucleic acid molecule, or a fusion polypeptide encoded by the fusion nucleic acid molecule, wherein: (a) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or (b) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2, wherein detection of the fusion nucleic acid molecule or fusion polypeptide encoded by the fusion nucleic acid molecule in the sample identifies the individual as one who may benefit from a treatment comprising an anti-cancer therapy.

In another aspect, provided herein is a method of identifying one or more treatment options for an individual having a cancer, the method comprising: (a) detecting in a sample from the individual a fusion nucleic acid molecule, or a fusion polypeptide encoded by the fusion nucleic acid molecule, wherein: (i) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or (ii) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2; and (b) generating a report comprising one or more treatment options identified for the individual based at least in part on detection of the fusion nucleic acid molecule or fusion polypeptide encoded by the fusion nucleic acid molecule in the sample, wherein the one or more treatment options comprise an anti-cancer therapy.

In another aspect, provided herein is a method of identifying one or more treatment options for an individual having a cancer, the method comprising: (a) acquiring knowledge of a fusion nucleic acid molecule, or a fusion polypeptide encoded by the fusion nucleic acid molecule, in a sample from the individual, wherein: (i) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or (ii) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2; and (b) generating a report comprising one or more treatment options identified for the individual based at least in part on said knowledge, wherein the one or more treatment options comprise an anti-cancer therapy.

In another aspect, provided herein is a method of selecting a treatment for an individual having cancer, comprising acquiring knowledge of a fusion nucleic acid molecule, or a fusion polypeptide encoded by the fusion nucleic acid molecule, in a sample from the individual, wherein: (a) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or (b) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2; wherein responsive to the acquisition of said knowledge: (i) the individual is classified as a candidate to receive a treatment comprising an anti-cancer therapy; and/or (ii) the individual is identified as likely to respond to a treatment that comprises an anti-cancer therapy.

In another aspect, provided herein is a method of predicting survival of an individual having a cancer, comprising acquiring knowledge of a fusion nucleic acid molecule, or a fusion polypeptide encoded by the fusion nucleic acid molecule, in a sample from the individual, wherein: (a) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or (b) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2, wherein responsive to the acquisition of said knowledge, the individual is predicted to have longer survival when treated with a treatment comprising an anti-cancer therapy, as compared to survival of an individual whose cancer does not comprise the fusion nucleic acid molecule or fusion polypeptide encoded by the fusion nucleic acid molecule.

In another aspect, provided herein is a method of predicting survival of an individual having a cancer treated with a treatment comprising an anti-cancer therapy, the method comprising acquiring knowledge of a fusion nucleic acid molecule, or a fusion polypeptide encoded by the fusion nucleic acid molecule, in a sample from the individual, wherein: (a) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or (b) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2, wherein responsive to the acquisition of said knowledge, the individual is predicted to have longer survival when treated with a treatment comprising an anti-cancer therapy, as compared to an individual whose cancer does not exhibit the fusion nucleic acid molecule or fusion polypeptide encoded by the fusion nucleic acid molecule.

In another aspect, provided herein is a method of treating or delaying progression of cancer, comprising: (a) acquiring knowledge of a fusion nucleic acid molecule, or a fusion polypeptide encoded by the fusion nucleic acid molecule, in a sample from an individual having a cancer, wherein: (i) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or (ii) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2; and (b) responsive to said knowledge, administering to the individual an effective amount of a treatment that comprises an anti-cancer therapy.

In another aspect, provided herein is a method of treating or delaying progression of cancer, comprising administering to an individual having cancer an effective amount of a treatment that comprises an anti-cancer therapy, wherein the anti-cancer therapy is administered responsive to acquiring knowledge of a fusion nucleic acid molecule, or a fusion polypeptide encoded by the fusion nucleic acid molecule, in a sample from the individual, wherein: (a) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or (b) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2.

In another aspect, provided herein is a method of monitoring, evaluating or screening an individual having a cancer, comprising acquiring knowledge of a fusion nucleic acid molecule, or a fusion polypeptide encoded by the fusion nucleic acid molecule, in a sample from the individual, wherein: (a) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or (b) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2; wherein responsive to the acquisition of said knowledge, the individual is predicted to have acquired resistance to a prior anti-cancer therapy administered to the individual, the individual is predicted to respond to an anti-cancer therapy, and/or the individual is predicted to have poor prognosis, as compared to an individual whose cancer does not comprise the fusion nucleic acid molecule or fusion polypeptide encoded by the fusion nucleic acid molecule.

In another aspect, provided herein is a method of assessing a fusion nucleic acid molecule or a fusion polypeptide in a cancer in an individual, the method comprising: (a) detecting in a sample from the individual a fusion nucleic acid molecule, or a fusion polypeptide encoded by the fusion nucleic acid molecule, wherein: (i) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or (ii) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2; and (b) providing an assessment of the fusion nucleic acid molecule or fusion polypeptide encoded by the fusion nucleic acid molecule.

In another aspect, provided herein is a method of detecting a fusion nucleic acid molecule or a fusion polypeptide, the method comprising detecting in a sample from an individual having a cancer a fusion nucleic acid molecule, or a fusion polypeptide encoded by the fusion nucleic acid molecule, wherein: (a) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or (b) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2.

In another aspect, provided herein is a method of detecting the presence or absence of a cancer in an individual, the method comprising: (a) detecting the presence or absence of a cancer in a sample from the individual; and (b) detecting in a sample from the individual the presence or absence of a fusion nucleic acid molecule, or a fusion polypeptide encoded by the fusion nucleic acid molecule, wherein: (i) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or (ii) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2. In some embodiments, the method comprises detecting the presence of the cancer in a sample from the individual. In some embodiments, the method comprises detecting the presence of the fusion nucleic acid molecule, or the fusion polypeptide encoded by the fusion nucleic acid molecule, in a sample from the individual.

In another aspect, provided herein is a method for monitoring progression or recurrence of a cancer in an individual, the method comprising: (a) detecting, in a first sample obtained from the individual at a first time point, the presence or absence of a fusion nucleic acid molecule, or a fusion polypeptide encoded by the fusion nucleic acid molecule; (b) detecting, in a second sample obtained from the individual at a second time point after the first time point, the presence or absence of a fusion nucleic acid molecule, or a fusion polypeptide encoded by the fusion nucleic acid molecule; and (c) providing an assessment of cancer progression or cancer recurrence in the individual based, at least in part, on the presence or absence of the fusion nucleic acid molecule or fusion polypeptide encoded by the fusion nucleic acid molecule in the first sample and/or in the second sample; wherein: (i) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or (ii) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2. In some embodiments, the presence of the fusion nucleic acid molecule or fusion polypeptide encoded by the fusion nucleic acid molecule in the first sample and/or in the second sample identifies the individual as having increased risk of cancer progression or cancer recurrence. In some embodiments, the method further comprises selecting a treatment, administering a treatment, adjusting a treatment, adjusting the dose of a treatment, or applying a treatment to the individual based, at least in part, on detecting the presence of the fusion nucleic acid molecule or fusion polypeptide encoded by the fusion nucleic acid molecule in the first sample and/or in the second sample, wherein the treatment comprises an anti-cancer therapy.

In another aspect, provided herein is a method of detecting a fusion nucleic acid molecule, the method comprising: (a) providing a plurality of nucleic acid molecules obtained from a sample from an individual having a cancer, wherein the plurality of nucleic acid molecules comprises nucleic acid molecules corresponding to a fusion nucleic acid molecule, wherein: (i) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or (ii) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2; (b) optionally, ligating one or more adapters onto one or more nucleic acid molecules from the plurality of nucleic acid molecules; (c) optionally, amplifying the one or more ligated nucleic acid molecules from the plurality of nucleic acid molecules; (d) optionally, capturing amplified nucleic acid molecules from the amplified nucleic acid molecules; (e) sequencing, by a sequencer, the captured nucleic acid molecules to obtain a plurality of sequence reads that represent the captured nucleic acid molecules, wherein one or more of the plurality of sequence reads correspond to the fusion nucleic acid molecule; (f) analyzing the plurality of sequence reads; and (g) based on the analysis, detecting the presence or absence of the fusion nucleic acid molecule in the sample. In some embodiments, the method further comprises receiving, at one or more processors, sequence read data for the plurality of sequence reads. In some embodiments, the analyzing the plurality of sequence reads comprises identifying, using the one or more processors, the presence or absence of sequence reads corresponding to the fusion nucleic acid molecule. In some embodiments, the amplified nucleic acid molecules are captured by hybridization with one or more bait molecules.

In another aspect, provided herein is a method of detecting a fusion nucleic acid molecule, the method comprising: (a) providing a sample from an individual having a cancer, wherein the sample comprises a plurality of nucleic acid molecules; (b) preparing a nucleic acid sequencing library from the plurality of nucleic acid molecules in the sample; (c) amplifying said library; (d) selectively enriching for one or more nucleic acid molecules comprising nucleotide sequences corresponding to a fusion nucleic acid molecule in said library to produce an enriched sample, wherein: (i) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or (ii) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2; (e) sequencing the enriched sample, thereby producing a plurality of sequence reads; (f) analyzing the plurality of sequence reads for the presence of the fusion nucleic acid molecule; (g) detecting, based on the analyzing step, the presence or absence of the fusion nucleic acid molecule in the sample from the individual.

In some embodiments, which may be combined with any of the preceding aspects or embodiments, the plurality of nucleic acid molecules comprises a mixture of cancer nucleic acid molecules and non-cancer nucleic acid molecules. In some embodiments, the cancer nucleic acid molecules are derived from a tumor portion of a heterogeneous tissue biopsy sample, and the non-cancer nucleic acid molecules are derived from a normal portion of the heterogeneous tissue biopsy sample. In some embodiments, the sample comprises a liquid biopsy sample, and wherein the cancer nucleic acid molecules are derived from a circulating tumor DNA (ctDNA) fraction of the liquid biopsy sample, and the non-cancer nucleic acid molecules are derived from a non-tumor and/or cell-free DNA (cfDNA) fraction of the liquid biopsy sample. In some embodiments, the one or more adapters comprise amplification primers, flow cell adaptor sequences, substrate adapter sequences, or sample index sequences. In some embodiments, the selectively enriching comprises: (a) combining one or more bait molecules with the library, thereby hybridizing the one or more bait molecules to one or more nucleic acid molecules comprising nucleotide sequences corresponding to the fusion nucleic acid molecule and producing nucleic acid hybrids; and (b) isolating the nucleic acid hybrids to produce the enriched sample. In some embodiments, the captured nucleic acid molecules are captured from the amplified nucleic acid molecules by hybridization to one or more bait molecules. In some embodiments, the amplifying comprises performing a polymerase chain reaction (PCR) amplification technique, a non-PCR amplification technique, or an isothermal amplification technique. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the sequencing comprises a massively parallel sequencing technique, and the massively parallel sequencing technique comprises next generation sequencing (NGS). In some embodiments, the sequencer comprises a next generation sequencer. In some embodiments, the method further comprises generating a genomic profile for the individual, based, at least in part, on detecting the presence or absence of the fusion nucleic acid molecule. In some embodiments, the genomic profile for the individual further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the genomic profile for the individual further comprises results from a nucleic acid sequencing-based test. In some embodiments, the method further comprises selecting a treatment, administering a treatment, or applying a treatment to the individual based on the generated genomic profile, wherein the treatment comprises an anti-cancer therapy. In some embodiments, the method further comprises generating a report indicating the presence or absence of the fusion nucleic acid molecule in the sample. In some embodiments, the method further comprises generating, by the one or more processors, a report indicating the presence or absence of the fusion nucleic acid molecule in the sample. In some embodiments, the method further comprises transmitting the report to a healthcare provider. In some embodiments, the report is transmitted via a computer network or a peer-to-peer connection.

In another aspect, provided herein is a method of identifying a candidate treatment for a cancer in an individual in need thereof, comprising performing DNA sequencing on a sample obtained from the individual to determine a sequencing mutation profile on a group of genes comprising one or more of ALK, BRAF, EGFR, ERBB2, FGFR1, FGFR2, FGFR3, MET, NTRK1, RAF1, RET, or ROS1, or any combination thereof, wherein the sequencing mutation profile identifies the presence or absence of a fusion nucleic acid molecule, wherein: (a) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or (b) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2. In some embodiments, the candidate treatment comprises an anti-cancer therapy. In some embodiments, the presence of the fusion nucleic acid molecule in the sample identifies the individual as one who may benefit from a treatment comprising an anti-cancer therapy. In some embodiments, the presence of the fusion nucleic acid molecule in the sample predicts the individual to have longer survival when treated with a treatment comprising an anti-cancer therapy, as compared to survival of an individual whose cancer does not comprise the fusion nucleic acid molecule. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the sequencing comprises a massively parallel sequencing technique, and the massively parallel sequencing technique comprises next generation sequencing (NGS). In some embodiments, the sequencing mutation profile identifies the presence or absence of a fragment of the fusion nucleic acid molecule comprising a breakpoint or fusion junction.

In another aspect, provided herein is a method of treating or delaying progression of cancer, comprising: (a) detecting in a sample from an individual having a cancer a fusion nucleic acid molecule, or a fusion polypeptide encoded by the fusion nucleic acid molecule, wherein: (i) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or (ii) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2; and (b) administering to the individual an effective amount of a treatment that comprises an anti-cancer therapy.

In some embodiments, which may be combined with any of the preceding aspects or embodiments, the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, comprising or resulting from a Breakpoint 1 and/or a Breakpoint 2 corresponding to the ALK, BRAF, EGFR, ERBB2, FGFR1, FGFR2, FGFR3, MET, RAF1, RET, or ROS1 fusion nucleic acid molecule as listed in Table 3.

In some embodiments, which may be combined with any of the preceding aspects or embodiments, the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, and wherein the cancer is a carcinoma, a sarcoma, a lymphoma, a leukemia, a myeloma, a germ cell cancer, or a blastoma.

In some embodiments, which may be combined with any of the preceding aspects or embodiments, the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, and wherein the cancer is a solid tumor.

In some embodiments, which may be combined with any of the preceding aspects or embodiments, the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, and wherein the cancer is a hematologic malignancy.

In some embodiments, which may be combined with any of the preceding aspects or embodiments, the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, and wherein the cancer is a B cell cancer, a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In some embodiments, which may be combined with any of the preceding aspects or embodiments, the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, and wherein the cancer is appendix adenocarcinoma, bladder adenocarcinoma, bladder urothelial (transitional cell) carcinoma, breast cancer not otherwise specified (NOS), breast carcinoma NOS, breast invasive ductal carcinoma (IDC), breast invasive lobular carcinoma (ILC), cervix squamous cell carcinoma (SCC), colon adenocarcinoma (CRC), esophagus adenocarcinoma, esophagus carcinoma NOS, esophagus squamous cell carcinoma (SCC), eye intraocular melanoma, gallbladder adenocarcinoma, gastroesophageal junction adenocarcinoma, intra-hepatic cholangiocarcinoma, kidney cancer NOS, liver hepatocellular carcinoma (HCC), lung cancer NOS, lung adenocarcinoma, lung large cell carcinoma, lung non-small cell lung carcinoma (NSCLC) NOS, lung small cell undifferentiated carcinoma, lung squamous cell carcinoma (SCC), ovary cancer NOS, pancreas cancer NOS, pancreas ductal adenocarcinoma, pancreatobiliary carcinoma, prostate cancer NOS, prostate acinar adenocarcinoma, prostate ductal adenocarcinoma, rectum adenocarcinoma (CRC), skin melanoma, small intestine adenocarcinoma, soft tissue sarcoma NOS, stomach adenocarcinoma NOS, unknown primary cancer NOS, unknown primary adenocarcinoma, unknown primary carcinoma (CUP) NOS, unknown primary neuroendocrine tumor, unknown primary squamous cell carcinoma (SCC), or uterus endometrial adenocarcinoma NOS.

In some embodiments, which may be combined with any of the preceding aspects or embodiments, the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, and the cancer is the cancer corresponding to the ALK, BRAF, EGFR, ERBB2, FGFR1, FGFR2, FGFR3, MET, RAF1, RET, or ROS1 fusion nucleic acid molecule as listed in Table 4.

In some embodiments, which may be combined with any of the preceding aspects or embodiments, the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, the cancer is the cancer corresponding to the ALK, BRAF, EGFR, ERBB2, FGFR1, FGFR2, FGFR3, MET, RAF1, RET, or ROS1 fusion nucleic acid molecule as listed in Table 5, and the fusion nucleic acid molecule comprises or results from a Breakpoint 1 and/or a Breakpoint 2 corresponding to the ALK, BRAF, EGFR, ERBB2, FGFR1, FGFR2, FGFR3, MET, RAF1, RET, or ROS1 fusion nucleic acid molecule as listed in Table 5.

In some embodiments, which may be combined with any of the preceding aspects or embodiments, the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2; and the fusion nucleic acid molecule comprises or results from a Breakpoint 1 and/or a Breakpoint 2 corresponding to the ALK, BRAF, ERBB2, FGFR1, FGFR2, FGFR3, MET, NTRK1, RAF1, RET, or ROS1 fusion nucleic acid molecule as listed in Table 6.

In some embodiments, which may be combined with any of the preceding aspects or embodiments, the fusion polypeptide encoded by the fusion nucleic acid molecule is oncogenic. In some embodiments, which may be combined with any of the preceding aspects or embodiments, the fusion polypeptide encoded by the fusion nucleic acid molecule promotes cancer cell survival, angiogenesis, cancer cell proliferation, and any combination thereof.

In some embodiments, which may be combined with any of the preceding aspects or embodiments, the anti-cancer therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for cancer comprising the fusion nucleic acid molecule or the fusion polypeptide encoded by the fusion nucleic acid molecule, a treatment for cancer being tested in a clinical trial, a targeted therapy, a treatment being tested in a clinical trial for cancer comprising the fusion nucleic acid molecule or fusion polypeptide encoded by the fusion nucleic acid molecule, or any combination thereof. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy. In some embodiments, the nucleic acid inhibits the expression of the fusion nucleic acid molecule or fusion polypeptide encoded by the fusion nucleic acid molecule. In some embodiments, the nucleic acid comprises a double-stranded RNA (dsRNA), a small interfering RNA (siRNA), or a small hairpin RNA (shRNA).

In some embodiments, which may be combined with any of the preceding aspects or embodiments, the anti-cancer therapy is a kinase inhibitor. In some embodiments, the kinase inhibitor is a multi-kinase inhibitor or an ALK-, BRAF-, EGFR-, ERBB2-, FGFR1-, FGFR2-, FGFR3-, MET-, RAF1-, NTRK1-, RET-, or ROS1-specific inhibitor.

In some embodiments, which may be combined with any of the preceding aspects or embodiments, the method further comprises acquiring knowledge of or detecting in a sample from the individual a base substitution, a short insertion/deletion (indel), a copy number alteration, or a genomic rearrangement in one or more genes.

In some embodiments, which may be combined with any of the preceding aspects or embodiments, the individual has received a prior anti-cancer treatment or is being treated with an anti-cancer treatment. In some embodiments, the fusion nucleic acid molecule, and/or the fusion polypeptide encoded by the fusion nucleic acid molecule, confers resistance of the cancer to the anti-cancer treatment. In some embodiments, the anti-cancer treatment is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for cancer being tested in a clinical trial, an immunotherapy, a chemotherapy, a targeted therapy, or any combination thereof. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy. In some embodiments, the nucleic acid comprises a double-stranded RNA (dsRNA), a small interfering RNA (siRNA), or a small hairpin RNA (shRNA).

In some embodiments, which may be combined with any of the preceding aspects or embodiments, the fusion nucleic acid molecule is an ALK fusion nucleic acid molecule as listed in any of Tables 1-6. In some embodiments, the ALK fusion nucleic acid molecule encodes an ALK fusion polypeptide. In some embodiments, the encoded ALK fusion polypeptide comprises an ALK kinase domain, or a fragment of an ALK kinase domain having ALK kinase activity. In some embodiments, the encoded ALK fusion polypeptide has ALK kinase activity, optionally wherein the ALK kinase activity is constitutive. In some embodiments, the encoded ALK fusion polypeptide is oncogenic. In some embodiments, the encoded ALK fusion polypeptide promotes cancer cell survival, angiogenesis, cancer cell proliferation, and any combination thereof. In some embodiments, the method further comprises acquiring knowledge of or detecting, in a sample from the individual, the presence of: (a) a mutation in an EGFR gene; optionally wherein the mutation is a deletion of exon 19 of EGFR or a portion thereof, a mutation resulting in an L858R, R748K, T790M, C797S, and/or D761N amino acid substitution in an encoded EGFR polypeptide, an EGFR gene amplification, or any combination thereof; (b) a mutation in a BRAF gene; optionally wherein the mutation is a mutation resulting in a V600E amino acid substitution in an encoded BRAF polypeptide; (c) a mutation in an NRAS gene; optionally wherein the mutation is a mutation resulting in a Q61H amino acid substitution in an encoded NRAS polypeptide; (d) a mutation in a MET gene; optionally wherein the mutation is a MET gene amplification, a mutation resulting in a D1228H amino acid substitution in an encoded MET polypeptide, or both; (e) a mutation in an NF1 gene; optionally wherein the mutation is an NF1 truncation; (f) a wild type KRAS gene, or a mutation in a KRAS gene; optionally wherein the mutation results in a G12V and/or A146P amino acid substitution in an encoded KRAS polypeptide; (g) a mutation in a MAP2K1 gene; optionally wherein the mutation is a mutation resulting in a I103_K104del mutation in an encoded MAP2K1 polypeptide; (h) an ALK mutation; optionally wherein the ALK mutation is an ALK resistance mutation, and optionally wherein the ALK resistance mutation results in a G1269A, G1202R, 11171S, 11171T, L1196M, T1151M, S1206Y, 11171N, D1203N, F1174C, L1152R, F1174L, L1198F, C1156Y, T1151_L1152insT, V1180L, G1202L, and/or S1206A amino acid substitution in an encoded ALK polypeptide, or any combination thereof; or any combination of (a)-(h). In some embodiments, the method further comprises acquiring knowledge of or detecting, in a sample from the individual, the presence of a mutation in an EGFR gene, optionally wherein the mutation results in an L858R amino acid substitution in an encoded EGFR polypeptide; wherein the ALK fusion nucleic acid molecule is an ALK-PLEKHA7 fusion nucleic acid molecule as listed in Tables 2 or 6. In some embodiments, the cancer is a non-small cell lung carcinoma (NSCLC). In some embodiments, which may be combined with any of the preceding aspects or embodiments, the individual was previously treated for cancer with erlotinib, afatinib, and/or osimertinib. In some embodiments, the individual exhibited a partial response to treatment with erlotinib; and/or wherein the individual exhibited a partial response to treatment with osimertinib. In some embodiments, the fusion nucleic acid molecule, and/or the fusion polypeptide encoded by the fusion nucleic acid molecule, confers resistance of the cancer to an EGFR-targeted anti-cancer therapy, optionally wherein the EGFR-targeted anti-cancer therapy is a first-, second-, or third-generation EGFR tyrosine kinase inhibitor. In some embodiments, the fusion nucleic acid molecule, and/or the fusion polypeptide encoded by the fusion nucleic acid molecule, confers resistance of the cancer to an EGFR-targeted anti-cancer therapy, optionally wherein the EGFR-targeted anti-cancer therapy is cetuximab, panitumumab, lapatinib, gefitinib, vandetanib, dacomitinib, icotinib, osimertinib (AZD9291), afatanib, olmutinib, EGF816 (nazartinib), avitinib (AC0010), rociletinib (CO-1686), BMS-690514, YH5448, PF-06747775, ASP8273, PF299804, AP26113, or erlotinib. In some embodiments, the fusion nucleic acid molecule, and/or the fusion polypeptide encoded by the fusion nucleic acid molecule, confers resistance of the cancer to an NF1-targeted anti-cancer therapy. In some embodiments, the method further comprises acquiring knowledge of or detecting, in a sample from the individual, the presence of: (a) an ALK resistance mutation; optionally wherein the ALK resistance mutation results in a V1180L, 11171N, L1196M, D1203N, or 11171T amino acid substitution in an encoded ALK polypeptide, or any combination thereof; and/or (b) a mutation in a KRAS gene; optionally wherein the mutation results in a G12V amino acid substitution in an encoded KRAS polypeptide; wherein the ALK fusion nucleic acid molecule is an ALK-HIP1 fusion nucleic acid molecule as listed in Tables 2 or 6. In some embodiments, the sample comprises one or more ALK gene mutations that result in a V1180L and I1171N amino acid substitution in an encoded ALK polypeptide; or a D1203N and I1171T amino acid substitution in an encoded ALK polypeptide. In some embodiments, the sample comprises a mutation in a KRAS gene; optionally wherein the mutation results in a G12V amino acid substitution in an encoded KRAS polypeptide. In some embodiments, the cancer is an unknown primary carcinoma. In some embodiments, the anti-cancer therapy is an ALK-targeted therapy. In some embodiments, the ALK-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for ALK-positive or ALK-rearranged cancer, an ALK-targeted therapy being tested in a clinical trial, a treatment for ALK-positive or ALK-rearranged cancer being tested in a clinical trial, or any combination thereof. In some embodiments, the ALK-targeted therapy is a kinase inhibitor. In some embodiments, the ALK-targeted therapy is a tyrosine kinase inhibitor. In some embodiments, the ALK-targeted therapy is a multi-kinase inhibitor or an ALK-specific inhibitor. In some embodiments, the kinase inhibitor inhibits a kinase activity of an ALK polypeptide. In some embodiments, the ALK-targeted therapy comprises one or more of crizotinib, alectinib, ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, TAE684 (NVP-TAE684), CT-707, WX-0593, alkotinib, SIM1803-1A, PLB1003, SAF-189s, PF03446962, TQ-B3101, APG-2449, X-376, CEP-28122, and GSK1838705A. In some embodiments, the nucleic acid inhibits the expression of the ALK fusion nucleic acid molecule or fusion polypeptide encoded by the fusion nucleic acid molecule. In some embodiments, the nucleic acid is a double-stranded RNA (dsRNA), a small interfering RNA (siRNA), or a small hairpin RNA (shRNA). In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

In some embodiments, which may be combined with any of the preceding aspects or embodiments, the fusion nucleic acid molecule is a BRAF fusion nucleic acid molecule as listed in any of Tables 1-6. In some embodiments, the BRAF fusion nucleic acid molecule encodes a BRAF fusion polypeptide. In some embodiments, the encoded BRAF fusion polypeptide comprises a BRAF kinase domain, or a fragment of a BRAF kinase domain having BRAF kinase activity. In some embodiments, the encoded BRAF fusion polypeptide has BRAF kinase activity, optionally wherein the BRAF kinase activity is constitutive. In some embodiments, the encoded BRAF fusion polypeptide is oncogenic. In some embodiments, the encoded BRAF fusion polypeptide promotes cancer cell survival, angiogenesis, cancer cell proliferation, and any combination thereof. In some embodiments, the method further comprises acquiring knowledge of or detecting, in a sample from the individual, the presence of: (a) a mutation in an EGFR gene; optionally wherein the mutation is an EGFR gene amplification, and/or a mutation resulting in a V441G, S492R, and/or G465E/R amino acid substitution in an encoded EGFR polypeptide; (b) a wild type KRAS gene, or a mutation in a KRAS gene; optionally wherein the mutation results in a G12F, G12V, G12C, G13D and/or Q61H amino acid substitution in an encoded KRAS polypeptide; (c) a mutation in an NRAS gene; optionally wherein the mutation results in a G13D and/or Q61K/L amino acid substitution in an encoded NRAS polypeptide; (d) a mutation in a MET gene, optionally where the mutation is a MET gene amplification; (e) a mutation in a MAP2K1 gene, optionally wherein the mutation results in a Q58del or E102_I103del mutation and/or 1111T or K57T amino acid substitution in an encoded MAP2K1 polypeptide; (f) a mutation in a MAP2K2 gene, optionally wherein the mutation results in a F57V amino acid substitution in an encoded MAP2K2 polypeptide; (g) a mutation in an NF1 gene, optionally wherein the mutation is a F945fs*9 mutation; (h) a mutation in a BRAF gene, optionally wherein the mutation results in a V600E amino acid substitution in an encoded BRAF polypeptide; and/or (i) a mutation in an HRAS gene, optionally wherein the mutation results in a Q61L amino acid substitution in an encoded HRAS polypeptide. In some embodiments, the method further comprises acquiring knowledge of or detecting, in a sample from the individual, the presence of: an EGFR gene amplification; and a wild type KRAS gene, or a KRAS gene mutation resulting in a G12F and/or Q61H amino acid substitution in an encoded KRAS polypeptide; wherein the BRAF fusion nucleic acid molecule is a BRAF-SND1 fusion nucleic acid molecule listed in Tables 2 or 6. In some embodiments, the method further comprises acquiring knowledge of or detecting, in a sample from the individual, the presence of: a mutation in an EGFR gene resulting in a V441G and/or G465E/R amino acid substitution in an encoded EGFR polypeptide; a wild type KRAS gene, or a KRAS gene mutation resulting in a G12C amino acid substitution in an encoded KRAS polypeptide; a mutation in an NRAS gene resulting in a G13D and/or Q61K amino acid substitution in an encoded NRAS polypeptide; and a MET gene amplification, wherein the BRAF fusion nucleic acid molecule is a BRAF-ZC3HAV1 fusion nucleic acid molecule listed in Tables 2 or 6. In some embodiments, the method further comprises acquiring knowledge of or detecting, in a sample from the individual, the presence of: a mutation in an EGFR gene resulting in an S492R amino acid substitution in an encoded EGFR polypeptide; a wild type KRAS gene, or a mutation in a KRAS gene; optionally wherein the mutation results in a G12V and/or Q61H amino acid substitution in an encoded KRAS polypeptide; a mutation in an NRAS gene resulting in a Q61K/L amino acid substitution in an encoded NRAS polypeptide; a mutation in a MAP2K1 gene resulting in a Q58del mutation and/or I1111T amino acid substitution in an encoded MAP2K1 polypeptide; a mutation in a MAP2K2 gene resulting in a F57V amino acid substitution in an encoded MAP2K2 polypeptide; and a F945fs*9 mutation in an NF1 gene, wherein the BRAF fusion nucleic acid molecule is an BRAF-MKRN1 fusion nucleic acid molecule listed in Tables 2 or 6. In some embodiments, the method further comprises acquiring knowledge of or detecting, in a sample from the individual, the presence of: a mutation in a KRAS gene resulting in a G13D amino acid substitution in an encoded KRAS polypeptide; wherein the BRAF fusion nucleic acid molecule is a BRAF-DENND2A fusion nucleic acid molecule listed in Tables 2 or 6. In some embodiments, the cancer was previously treated with folinic acid, fluorouracil (5-FU), and oxaliplatin (FOLFOX); 5-FU; folinic acid, 5-FU, and irinotecan (FOLFIRI); and/or regorafenib. In some embodiments, the method further comprises acquiring knowledge of or detecting, in a sample from the individual, the presence of: a wild type KRAS gene; and a mutation in an NRAS gene resulting in a Q61K amino acid substitution in an encoded NRAS polypeptide; wherein the BRAF fusion nucleic acid molecule is an BRAF-TRIM24 fusion nucleic acid molecule listed in Tables 2 or 6. In some embodiments, the cancer is a colorectal cancer. In some embodiments, the method further comprises acquiring knowledge of or detecting, in a sample from the individual, the presence of: a mutation in a BRAF gene resulting in an V600E amino acid substitution in an encoded BRAF polypeptide; a mutation in an EGFR gene resulting in a S492R and/or V441G amino acid substitution in an encoded EGFR polypeptide; a wild type KRAS gene; a mutation in an HRAS gene resulting in an Q61L amino acid substitution in an encoded HRAS polypeptide; a mutation in a MAP2K1 gene resulting in a E102_I103del mutation and/or a K57T amino acid substitution in an encoded MAP2K1 polypeptide; and a mutation in an NRAS gene resulting in a Q61K amino acid substitution in an encoded NRAS polypeptide, wherein the BRAF fusion nucleic acid molecule is a BRAF-GOLGA3 fusion nucleic acid molecule as listed in any of Tables 1 and 3-5. In some embodiments, the cancer is a colorectal cancer. In some embodiments, the cancer was previously treated with 5-FU; folinic acid, 5-FU, and irinotecan (FOLFIRI) in combination with bevacizumab; FOLFIRI in combination with cetuximab; folinic acid, 5-FU, and oxaliplatin (FOLFOX) in combination with bevacizumab; and/or pembrolizumab in combination with regorafenib. In some embodiments, the method further comprises acquiring knowledge of or detecting, in a sample from the individual, the presence of: a mutation in a KRAS gene resulting in a G12C and/or G13D amino acid substitution in an encoded KRAS polypeptide; a mutation in a MAP2K1 gene resulting in a E102_I103del mutation in an encoded MAP2K1 polypeptide; and a mutation in an NRAS gene resulting in an Q61K amino acid substitution in an encoded NRAS polypeptide, wherein the BRAF fusion nucleic acid molecule is an BRAF-AKAP9 fusion nucleic acid molecule as listed in Tables 2 or 6. In some embodiments, the cancer is a colorectal cancer. In some embodiments, the cancer was previously treated with adagrasib or adagrasib in combination with cetuximab. In some embodiments, the fusion nucleic acid molecule, and/or the fusion polypeptide encoded by the fusion nucleic acid molecule, confers resistance of the cancer to an EGFR-targeted anti-cancer therapy, optionally wherein the EGFR-targeted anti-cancer therapy is a first-, second-, or third-generation EGFR tyrosine kinase inhibitor. In some embodiments, the fusion nucleic acid molecule, and/or the fusion polypeptide encoded by the fusion nucleic acid molecule, confers resistance of the cancer to an EGFR-targeted anti-cancer therapy, optionally wherein the EGFR-targeted anti-cancer therapy is cetuximab, panitumumab, lapatinib, gefitinib, vandetanib, dacomitinib, icotinib, osimertinib (AZD9291), afatanib, olmutinib, EGF816 (nazartinib), avitinib (AC0010), rociletinib (CO-1686), BMS-690514, YH5448, PF-06747775, ASP8273, PF299804, AP26113, or erlotinib. In some embodiments, the anti-cancer therapy is a BRAF-targeted therapy. In some embodiments, the BRAF-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for BRAF-rearranged cancer, a BRAF-targeted therapy being tested in a clinical trial, a treatment for BRAF-rearranged cancer being tested in a clinical trial, or any combination thereof. In some embodiments, the BRAF-targeted therapy is a kinase inhibitor. In some embodiments, the BRAF-targeted therapy is a serine/threonine kinase inhibitor. In some embodiments, the BRAF-targeted therapy is a multi-kinase inhibitor or a BRAF-specific inhibitor. In some embodiments, the kinase inhibitor inhibits a kinase activity of a BRAF polypeptide. In some embodiments, the BRAF-targeted therapy comprises one or more of sorafenib, PLX4720, PLX-3603, dabrafenib (GSK2118436), encorafenib (LGX818), GDC-0879, RAF265, XL281, ARQ736, BAY73-4506, vemurafenib, cobimetinib, binimetinib, regorafenib, selumetinib, trametinib, or BAY 43-9006. In some embodiments, the nucleic acid inhibits the expression of the BRAF fusion nucleic acid molecule or fusion polypeptide encoded by the fusion nucleic acid molecule. In some embodiments, the nucleic acid is a double-stranded RNA (dsRNA), a small interfering RNA (siRNA), or a small hairpin RNA (shRNA). In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

In some embodiments, which may be combined with any of the preceding aspects or embodiments, the fusion nucleic acid molecule is an EGFR fusion nucleic acid molecule as listed in any of Tables 1 and 3-5. In some embodiments, the EGFR fusion nucleic acid molecule encodes an EGFR fusion polypeptide. In some embodiments, the encoded EGFR fusion polypeptide comprises an EGFR kinase domain, or a fragment of an EGFR kinase domain having EGFR kinase activity. In some embodiments, the encoded EGFR fusion polypeptide has EGFR kinase activity, optionally wherein the EGFR kinase activity is constitutive. In some embodiments, the encoded EGFR fusion polypeptide is oncogenic. In some embodiments, the encoded EGFR fusion polypeptide promotes cancer cell survival, angiogenesis, cancer cell proliferation, and any combination thereof. In some embodiments, the method further comprises acquiring knowledge of or detecting, in a sample from the individual, the presence of: (a) a wild type KRAS gene, or a mutation in a KRAS gene; optionally wherein the mutation results in a G12A, and/or Q61H amino acid substitution in an encoded KRAS polypeptide; (b) a mutation in an NRAS gene; optionally wherein the mutation results in a G12D amino acid substitution in an encoded NRAS polypeptide; and/or (c) a mutation in a MAP2K1 gene, optionally wherein the mutation results in a E102_I103del mutation in an encoded MAP2K1 polypeptide. In some embodiments, the method further comprises acquiring knowledge of or detecting, in a sample from the individual, the presence of: a wild type KRAS gene, or a mutation in a KRAS gene resulting in a G12A, and/or Q61H amino acid substitution in an encoded KRAS polypeptide; a mutation in an NRAS gene resulting in a G12D amino acid substitution in an encoded NRAS polypeptide; and/or a mutation in a MAP2K1 gene resulting in a E102_I103del mutation in an encoded MAP2K1 polypeptide, wherein the fusion nucleic acid molecule is an EGFR-PDE7A fusion nucleic acid molecule listed in any of Tables 1 and 3-5. In some embodiments, the anti-cancer therapy is an EGFR-targeted therapy. In some embodiments, the EGFR-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for an EGFR-rearranged cancer, an EGFR-targeted therapy being tested in a clinical trial, a treatment for EGFR-rearranged cancer being tested in a clinical trial, or any combination thereof. In some embodiments, the EGFR-targeted therapy is a kinase inhibitor. In some embodiments, the EGFR-targeted therapy is a tyrosine kinase inhibitor. In some embodiments, the EGFR-targeted therapy is a multi-kinase inhibitor or an EGFR-specific inhibitor. In some embodiments, the kinase inhibitor inhibits a kinase activity of an EGFR polypeptide. In some embodiments, the EGFR-targeted therapy comprises one or more of cetuximab, panitumumab, lapatinib, gefitinib, vandetanib, dacomitinib, icotinib, osimertinib (AZD9291), afatanib, olmutinib, EGF816 (nazartinib), avitinib (AC0010), rociletinib (CO-1686), BMS-690514, YH5448, PF-06747775, ASP8273, PF299804, AP26113, necitumumab, or erlotinib. In some embodiments, the nucleic acid inhibits the expression of the EGFR fusion nucleic acid molecule or fusion polypeptide encoded by the fusion nucleic acid molecule. In some embodiments, the nucleic acid is a double-stranded RNA (dsRNA), a small interfering RNA (siRNA), or a small hairpin RNA (shRNA). In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

In some embodiments, which may be combined with any of the preceding aspects or embodiments, the fusion nucleic acid molecule is an ERBB2 fusion nucleic acid molecule as listed in any of Tables 1-6. In some embodiments, the ERBB2 fusion nucleic acid molecule encodes an ERBB2 fusion polypeptide. In some embodiments, the encoded ERBB2 fusion polypeptide comprises an ERBB2 kinase domain, or a fragment of an ERBB2 kinase domain having ERBB2 kinase activity. In some embodiments, the encoded ERBB2 fusion polypeptide has ERBB2 kinase activity, optionally wherein the ERBB2 kinase activity is constitutive. In some embodiments, the encoded ERBB2 fusion polypeptide is oncogenic. In some embodiments, the encoded ERBB2 fusion polypeptide promotes cancer cell survival, angiogenesis, cancer cell proliferation, and any combination thereof. In some embodiments, the anti-cancer therapy is an ERBB2-targeted therapy. In some embodiments, the ERBB2-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for an ERBB2-rearranged cancer, an ERBB2-targeted therapy being tested in a clinical trial, a treatment for ERBB2-rearranged cancer being tested in a clinical trial, or any combination thereof. In some embodiments, the ERBB2-targeted therapy is a kinase inhibitor. In some embodiments, the ERBB2-targeted therapy is a tyrosine kinase inhibitor. In some embodiments, the ERBB2-targeted therapy is a multi-kinase inhibitor or an ERBB2-specific inhibitor. In some embodiments, the kinase inhibitor inhibits a kinase activity of an ERBB2 polypeptide. In some embodiments, the ERBB2-targeted therapy comprises one or more of afatinib, TAK-285, neratinib, dacomitinib, BMS-690514, BMS-599626, pelitinib, CP-724714, lapatinib, TAK-165, ARRY-380, AZD8931, AV-203, AMG-888, MM-111, MM-121, MM-141, LJM716, REGN1400, MEHD7945A, RG7116, trastuzumab, trastuzumab emtansine (T-DM1), pertuzumab, or APC 8024. In some embodiments, the nucleic acid inhibits the expression of the ERBB2 fusion nucleic acid molecule or fusion polypeptide encoded by the fusion nucleic acid molecule. In some embodiments, the nucleic acid is a double-stranded RNA (dsRNA), a small interfering RNA (siRNA), or a small hairpin RNA (shRNA). In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

In some embodiments, which may be combined with any of the preceding aspects or embodiments, the fusion nucleic acid molecule is an FGFR1 fusion nucleic acid molecule as listed in any of Tables 1-6. In some embodiments, the FGFR1 fusion nucleic acid molecule encodes an FGFR1 fusion polypeptide. In some embodiments, the encoded FGFR1 fusion polypeptide comprises an FGFR1 kinase domain, or a fragment of an FGFR1 kinase domain having FGFR1 kinase activity. In some embodiments, the encoded FGFR1 fusion polypeptide has FGFR1 kinase activity, optionally wherein the FGFR1 kinase activity is constitutive. In some embodiments, the encoded FGFR1 fusion polypeptide is oncogenic. In some embodiments, the encoded FGFR1 fusion polypeptide promotes cancer cell survival, angiogenesis, cancer cell proliferation, and any combination thereof. In some embodiments, the anti-cancer therapy is an FGFR1-targeted therapy. In some embodiments, the FGFR1-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for an FGFR1-rearranged cancer, an FGFR1-targeted therapy being tested in a clinical trial, a treatment for FGFR1-rearranged cancer being tested in a clinical trial, or any combination thereof. In some embodiments, the FGFR1-targeted therapy is a kinase inhibitor. In some embodiments, the FGFR1-targeted therapy is a tyrosine kinase inhibitor. In some embodiments, the FGFR1-targeted therapy is a multi-kinase inhibitor or an FGFR1-specific inhibitor. In some embodiments, the kinase inhibitor inhibits a kinase activity of an FGFR1 polypeptide. In some embodiments, the FGFR1-targeted therapy comprises one or more of E3810 (lucitanib), AZD4547, Dovitinib (TKI258), Ponatinib, Derazantinib (ARQ 087), Nintendanib (BIBF1120), Rogaratinib (BAY 1163877), 3D185, SOMCL-085, brivanib (BMS582664), lenvatinib (E7080), orantinib (TSU-68), PRN1371, XL-228, AZ12908010 (AZ8010), Debio-1347 (CH5183284), FIIN-2, LY2874455, Infigratinib (BGJ398, NVP-BGJ398), Pemigatinib, Erdafitinib (JNJ-42756493), ASP5878, TAS-120, PRN1371, pazopanib, regorafenib, or PKC412. In some embodiments, the nucleic acid inhibits the expression of the FGFR1 fusion nucleic acid molecule or fusion polypeptide encoded by the fusion nucleic acid molecule. In some embodiments, the nucleic acid is a double-stranded RNA (dsRNA), a small interfering RNA (siRNA), or a small hairpin RNA (shRNA). In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

In some embodiments, which may be combined with any of the preceding aspects or embodiments, the fusion nucleic acid molecule is an FGFR2 fusion nucleic acid molecule as listed in any of Tables 1-6. In some embodiments, the FGFR2 fusion nucleic acid molecule encodes an FGFR2 fusion polypeptide. In some embodiments, the encoded FGFR2 fusion polypeptide comprises an FGFR2 kinase domain, or a fragment of an FGFR2 kinase domain having FGFR2 kinase activity. In some embodiments, the encoded FGFR2 fusion polypeptide has FGFR2 kinase activity, optionally wherein the FGFR2 kinase activity is constitutive. In some embodiments, the encoded FGFR2 fusion polypeptide is oncogenic. In some embodiments, the encoded FGFR2 fusion polypeptide promotes cancer cell survival, angiogenesis, cancer cell proliferation, and any combination thereof. In some embodiments, the method further comprises acquiring knowledge of or detecting, in a sample from the individual, the presence of an EGFR gene mutation; optionally wherein the EGFR gene mutation results in an L858R, L833V, and/or T790M amino acid substitution in an encoded EGFR polypeptide. In some embodiments, the individual has been previously treated for cancer with erlotinib. In some embodiments, the fusion nucleic acid molecule, and/or the fusion polypeptide encoded by the fusion nucleic acid molecule, confers resistance of the cancer to an EGFR-targeted anti-cancer therapy, optionally wherein the EGFR-targeted anti-cancer therapy is a first-, second-, or third-generation EGFR tyrosine kinase inhibitor. In some embodiments, the fusion nucleic acid molecule, and/or the encoded fusion polypeptide, confers resistance to an EGFR-targeted anti-cancer therapy, optionally wherein the EGFR-targeted anti-cancer therapy is cetuximab, panitumumab, lapatinib, gefitinib, vandetanib, dacomitinib, icotinib, osimertinib (AZD9291), afatanib, olmutinib, EGF816 (nazartinib), avitinib (AC00010), rociletinib (CO-1686), BMS-690514, YH5448, PF-06747775, ASP8273, PF299804, AP26113, or erlotinib. In some embodiments, the anti-cancer therapy is an FGFR2-targeted therapy. In some embodiments, the FGFR2-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for an FGFR2-rearranged cancer, an FGFR2-targeted therapy being tested in a clinical trial, a treatment for FGFR2-rearranged cancer being tested in a clinical trial, or any combination thereof. In some embodiments, the FGFR2-targeted therapy is a kinase inhibitor. In some embodiments, the FGFR2-targeted therapy is a tyrosine kinase inhibitor. In some embodiments, the FGFR2-targeted therapy is a multi-kinase inhibitor or an FGFR2-specific inhibitor. In some embodiments, the kinase inhibitor inhibits a kinase activity of an FGFR2 polypeptide. In some embodiments, the FGFR2-targeted therapy comprises one or more of E3810 (lucitanib), AZD4547, Dovitinib (TKI258), Ponatinib, Derazantinib (ARQ 087), Nintendanib (BIBF1120), Rogaratinib (BAY 1163877), 3D185, SOMCL-085, brivanib (BMS582664), lenvatinib (E7080), orantinib (TSU-68), PRN1371, XL-228, AZ12908010 (AZ8010), Debio-1347 (CH5183284), FIIN-2, LY2874455, Infigratinib (BGJ398, NVP-BGJ398), Pemigatinib, Erdafitinib, ASP5878, TAS-120, PRN1371, formononetin, R04383596, Ki23057, SU5402, RLY-4008, pazopanib, regorafenib, or PKC412. In some embodiments, the nucleic acid inhibits the expression of the FGFR2 fusion nucleic acid molecule or fusion polypeptide encoded by the fusion nucleic acid molecule. In some embodiments, the nucleic acid is a double-stranded RNA (dsRNA), a small interfering RNA (siRNA), or a small hairpin RNA (shRNA). In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

In some embodiments, which may be combined with any of the preceding aspects or embodiments, the fusion nucleic acid molecule is an FGFR3 fusion nucleic acid molecule as listed in any of Tables 1-6. In some embodiments, the FGFR3 fusion nucleic acid molecule encodes an FGFR3 fusion polypeptide. In some embodiments, the encoded FGFR3 fusion polypeptide comprises an FGFR3 kinase domain, or a fragment of an FGFR3 kinase domain having FGFR3 kinase activity. In some embodiments, the encoded FGFR3 fusion polypeptide has FGFR3 kinase activity, optionally wherein the FGFR3 kinase activity is constitutive. In some embodiments, the encoded FGFR3 fusion polypeptide is oncogenic. In some embodiments, the encoded FGFR3 fusion polypeptide promotes cancer cell survival, angiogenesis, cancer cell proliferation, and any combination thereof. In some embodiments, the method further comprises acquiring knowledge of or detecting, in a sample from the individual, the presence of: (a) a mutation in an EGFR gene; optionally wherein the mutation is a deletion of exon 19 of EGFR or a portion thereof, an EGFR gene amplification, or a mutation resulting in a T790M, C797G, V441G, G465R, E709K or L858R amino acid substitution in an encoded EGFR polypeptide, or any combination thereof; (b) a mutation in a BRAF gene; optionally wherein the mutation results in a V600E amino acid substitution in an encoded BRAF polypeptide; (c) a wild type KRAS gene, or a mutation in a KRAS gene; optionally wherein the mutation results in a Q61H amino acid substitution in an encoded KRAS polypeptide; (d) a mutation in an ESR1 gene; optionally wherein the mutation results in a Y537N and/or D538G amino acid substitution in an encoded ESR1 polypeptide; (e) a mutation in an AKT1 gene; optionally wherein the mutation results in an E17K amino acid substitution in an encoded AKT1 polypeptide; or any combination of (a)-(e). In some embodiments, the method further comprises acquiring knowledge of or detecting, in a sample from the individual, the presence of: (a) a mutation in an EGFR gene; optionally wherein the mutation is a deletion of exon 19 of EGFR or a portion thereof, an EGFR gene amplification, or a mutation resulting in a S492R, V441G, G465R, E709K or L858R amino acid substitution in an encoded EGFR polypeptide, or any combination thereof; (b) a wild type KRAS gene, or a mutation in a KRAS gene; optionally wherein the mutation results in a G12C, G13D, and/or Q61H amino acid substitution in an encoded KRAS polypeptide; (c) a mutation in an ESR1 gene; optionally wherein the mutation results in a Y537N and/or D538G amino acid substitution in an encoded ESR1 polypeptide; (d) a mutation in an AKT1 gene; optionally wherein the mutation results in an E17K amino acid substitution in an encoded AKT1 polypeptide; (e) a mutation in a BRAF gene; optionally wherein the mutation results in an V600E amino acid substitution in an encoded BRAF polypeptide; (f) a mutation in an HRAS gene; optionally wherein the mutation results in an Q61L amino acid substitution in an encoded HRAS polypeptide; (g) a mutation in a MAP2K1 gene; optionally wherein the mutation results in a E102_I103del mutation and/or a K57T amino acid substitution in an encoded MAP2K1 polypeptide; (h) a mutation in an NRAS gene; optionally wherein the mutation results in an Q61K amino acid substitution in an encoded NRAS polypeptide; or any combination of (a)-(h); wherein the FGFR3 fusion nucleic acid molecule is an FGFR3-TACC3 fusion nucleic acid molecule as listed in Tables 2 or 6. In some embodiments, the cancer is a colorectal cancer, a non-small cell lung cancer, or a breast cancer. In some embodiments, the sample comprises a deletion of exon 19 of EGFR or a portion thereof. In some embodiments, the sample comprises EGFR gene mutations resulting in an L858R and/or E709K amino acid substitution in an encoded EGFR polypeptide. In some embodiments, the individual was previously treated for cancer with afatinib and/or cetuximab. In some embodiments, the individual experienced stable disease during or after treatment with afatinib and cetuximab. In some embodiments, the method further comprises acquiring knowledge of or detecting, in a sample from the individual, the presence of: a mutation in a BRAF gene resulting in an V600E amino acid substitution in an encoded BRAF polypeptide; a mutation in an EGFR gene resulting in a S492R and/or V441G amino acid substitution in an encoded EGFR polypeptide; a wild type KRAS gene; a mutation in an HRAS gene resulting in an Q61L amino acid substitution in an encoded HRAS polypeptide; a mutation in a MAP2K1 gene resulting in a E102_I103del mutation and/or a K57T amino acid substitution in an encoded MAP2K1 polypeptide; and a mutation in an NRAS gene resulting in an Q61K amino acid substitution in an encoded NRAS polypeptide, wherein the FGFR3 fusion nucleic acid molecule is an FGFR3-TACC3 fusion nucleic acid molecule as listed in Tables 2 or 6. In some embodiments, the cancer is a colorectal cancer. In some embodiments, the cancer was previously treated with 5-FU; folinic acid, 5-FU, and irinotecan (FOLFIRI) in combination with bevacizumab; FOLFIRI in combination with cetuximab; folinic acid, 5-FU, and oxaliplatin (FOLFOX) in combination with bevacizumab; and/or pembrolizumab in combination with regorafenib. In some embodiments, the method further comprises acquiring knowledge of or detecting, in a sample from the individual, the presence of: a mutation in a KRAS gene resulting in a G12C and/or G13D amino acid substitution in an encoded KRAS polypeptide; a mutation in a MAP2K1 gene resulting in a E102_I103del mutation in an encoded MAP2K1 polypeptide; and a mutation in an NRAS gene resulting in a Q61K amino acid substitution in an encoded NRAS polypeptide, wherein the FGFR3 fusion nucleic acid molecule is an FGFR3-TACC3 fusion nucleic acid molecule as listed in Tables 2 or 6. In some embodiments, the cancer is a colorectal cancer. In some embodiments, the cancer was previously treated with adagrasib or adagrasib in combination with cetuximab. In some embodiments, the sample comprises an EGFR gene amplification, EGFR gene mutations resulting in a V441G and/or G465R amino acid substitution in an encoded EGFR polypeptide, and a KRAS gene mutation resulting in a Q61H amino acid substitution in an encoded KRAS polypeptide, and wherein the cancer is a colorectal cancer. In some embodiments, the sample comprises an EGFR gene amplification, EGFR gene mutations resulting in a V441G and/or G465R amino acid substitution in an encoded EGFR polypeptide, and a wild type KRAS gene, and wherein the cancer is a colorectal cancer. In some embodiments, the individual was previously treated for cancer with FOLFOXIRI (fluorouracil, leucovorin, oxaliplatin, and irinotecan), bevacizumab, and/or panitumumab. In some embodiments, the method further comprises acquiring knowledge of or detecting, in a sample from the individual, the presence of an SNRNP70-MET gene fusion. In some embodiments, the sample comprises ESR1 gene mutations resulting in a Y537N and/or D538G amino acid substitution in an encoded ESR1 polypeptide, and AKT1 gene mutations resulting in an E17K amino acid substitution in an encoded AKT1 polypeptide, and wherein the cancer is a breast cancer. In some embodiments, the cancer is estrogen receptor-positive (ER+) and/or progesterone receptor-positive (PR+). In some embodiments, the cancer was previously treated with everolimus, denosumab, and/or fulvestrant. In some embodiments, the fusion nucleic acid molecule, and/or the fusion polypeptide encoded by the fusion nucleic acid molecule, confers resistance of the cancer to hormonal anti-cancer therapy. In some embodiments, the method further comprises acquiring knowledge of or detecting, in a sample from the individual, the presence of (a) a mutation in an EGFR gene; optionally wherein the mutation is a deletion of exon 19 of EGFR or a portion thereof, or a mutation resulting in a T790M and/or C797G amino acid substitution in an encoded EGFR polypeptide, or any combination thereof; (b) a mutation in a BRAF gene; optionally wherein the mutation results in a V600E amino acid substitution in an encoded BRAF polypeptide; or both (a) and (b); wherein the FGFR3 fusion nucleic acid molecule is an FGFR3-ADD1 fusion nucleic acid molecule as listed in Tables 2 or 6. In some embodiments, the cancer is a non-small cell lung carcinoma (NSCLC). In some embodiments, the sample comprises a deletion of exon 19 of EGFR or a portion thereof, an EGFR gene mutation resulting in a T790M and/or C797G amino acid substitution in an encoded EGFR polypeptide, and a BRAF gene mutation resulting in a V600E amino acid substitution in an encoded BRAF polypeptide. In some embodiments, the individual was previously treated for cancer with osimertinib. In some embodiments, the individual experienced stable disease during or after treatment with osimertinib. In some embodiments, the fusion nucleic acid molecule, and/or the fusion polypeptide encoded by the fusion nucleic acid molecule, confers resistance to an EGFR-targeted anti-cancer therapy, optionally wherein the EGFR-targeted anti-cancer therapy is a first-, second-, or third-generation EGFR tyrosine kinase inhibitor. In some embodiments, the fusion nucleic acid molecule, and/or the fusion polypeptide encoded by the fusion nucleic acid molecule, confers resistance of the cancer to an EGFR-targeted anti-cancer therapy, optionally wherein the EGFR-targeted anti-cancer therapy is cetuximab, panitumumab, lapatinib, gefitinib, vandetanib, dacomitinib, icotinib, osimertinib (AZD9291), afatanib, olmutinib, EGF816 (nazartinib), avitinib (AC00010), rociletinib (CO-1686), BMS-690514, YH5448, PF-06747775, ASP8273, PF299804, AP26113, or erlotinib. In some embodiments, the anti-cancer therapy is an FGFR3-targeted therapy. In some embodiments, the FGFR3-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for an FGFR3-rearranged cancer, an FGFR3-targeted therapy being tested in a clinical trial, a treatment for FGFR3-rearranged cancer being tested in a clinical trial, or any combination thereof. In some embodiments, the FGFR3-targeted therapy is a kinase inhibitor. In some embodiments, the FGFR3-targeted therapy is a tyrosine kinase inhibitor. In some embodiments, the FGFR3-targeted therapy is a multi-kinase inhibitor or an FGFR3-specific inhibitor. In some embodiments, the kinase inhibitor inhibits a kinase activity of an FGFR3 polypeptide. In some embodiments, the FGFR3-targeted therapy comprises one or more of E3810 (lucitanib), AZD4547, Dovitinib (TKI258), Ponatinib, Derazantinib (ARQ 087), Nintendanib (BIBF1120), Rogaratinib (BAY 1163877), 3D185, SOMCL-085, brivanib (BMS582664), lenvatinib (E7080), orantinib (TSU-68), PRN1371, XL-228, AZ12908010 (AZ8010), Debio-1347 (CH5183284), FIIN-2, LY2874455, Infigratinib (BGJ398, NVP-BGJ398), Pemigatinib, Erdafitinib, ASP5878, TAS-120, PRN1371, PKC412, Vofatamab (B-70), pazopanib, or MFGR1877S. In some embodiments, the nucleic acid inhibits the expression of the FGFR3 fusion nucleic acid molecule or fusion polypeptide encoded by the fusion nucleic acid molecule. In some embodiments, the nucleic acid is a double-stranded RNA (dsRNA), a small interfering RNA (siRNA), or a small hairpin RNA (shRNA). In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

In some embodiments, which may be combined with any of the preceding aspects or embodiments, the fusion nucleic acid molecule is a MET fusion nucleic acid molecule as listed in any of Tables 1-6. In some embodiments, the MET fusion nucleic acid molecule encodes a MET fusion polypeptide. In some embodiments, the encoded MET fusion polypeptide comprises a MET kinase domain, or a fragment of a MET kinase domain having MET kinase activity. In some embodiments, the encoded MET fusion polypeptide has MET kinase activity, optionally wherein the MET kinase activity is constitutive. In some embodiments, the encoded MET fusion polypeptide is oncogenic. In some embodiments, the encoded MET fusion polypeptide promotes cancer cell survival, angiogenesis, cancer cell proliferation, and any combination thereof. In some embodiments, the method further comprises acquiring knowledge of or detecting, in a sample from the individual, the presence of: (a) a mutation in an EGFR gene; optionally wherein the mutation is an EGFR gene amplification, or a mutation resulting in a V441G and/or G465R amino acid substitution in an encoded EGFR polypeptide, or any combination thereof; and/or (b) a wild type KRAS gene, or a mutation in a KRAS gene; optionally wherein the mutation results in a Q61H amino acid substitution in an encoded KRAS polypeptide. In some embodiments, the method further comprises acquiring knowledge of or detecting, in a sample from the individual, the presence of: an EGFR gene amplification; EGFR gene mutations resulting in a V441G and/or G465R amino acid substitution in an encoded EGFR polypeptide; and a wild type KRAS gene, or a KRAS gene mutation resulting in a Q61H amino acid substitution in an encoded KRAS polypeptide; wherein the MET fusion nucleic acid molecule is a MET-SNRNP70 fusion nucleic acid molecule listed in any of Tables 1 and 3-5. In some embodiments, the method further comprises acquiring knowledge of or detecting, in a sample from the individual, the presence of an FGFR3-TACC3 gene fusion. In some embodiments, the individual was previously treated for cancer with FOLFOXIRI (fluorouracil, leucovorin, oxaliplatin, and irinotecan), bevacizumab, and/or panitumumab. In some embodiments, the method further comprises acquiring knowledge of or detecting, in a sample from the individual, the presence of an EGFR gene amplification, and a wild type KRAS gene, or a KRAS gene mutation resulting in a Q61H amino acid substitution in an encoded KRAS polypeptide; wherein the MET fusion nucleic acid molecule is a MET-CAPZA2 fusion nucleic acid molecule listed in Tables 2 or 6. In some embodiments, the cancer is a colorectal cancer. In some embodiments, the fusion nucleic acid molecule, and/or the fusion polypeptide encoded by the fusion nucleic acid molecule, confers resistance of the cancer to an EGFR-targeted anti-cancer therapy, optionally wherein the EGFR-targeted anti-cancer therapy is a first-, second-, or third-generation EGFR tyrosine kinase inhibitor. In some embodiments, the fusion nucleic acid molecule, and/or the fusion polypeptide encoded by the fusion nucleic acid molecule, confers resistance of the cancer to an EGFR-targeted anti-cancer therapy, optionally wherein the EGFR-targeted anti-cancer therapy is cetuximab, panitumumab, lapatinib, gefitinib, vandetanib, dacomitinib, icotinib, osimertinib (AZD9291), afatanib, olmutinib, EGF816 (nazartinib), avitinib (AC0010), rociletinib (CO-1686), BMS-690514, YH5448, PF-06747775, ASP8273, PF299804, AP26113, or erlotinib. In some embodiments, the anti-cancer therapy is a MET-targeted therapy. In some embodiments, the MET-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for a MET-rearranged cancer, a MET-targeted therapy being tested in a clinical trial, a treatment for MET-rearranged cancer being tested in a clinical trial, or any combination thereof. In some embodiments, the MET-targeted therapy is a kinase inhibitor. In some embodiments, the MET-targeted therapy is a tyrosine kinase inhibitor. In some embodiments, the MET-targeted therapy is a multi-kinase inhibitor or a MET-specific inhibitor. In some embodiments, the kinase inhibitor inhibits a kinase activity of a MET polypeptide. In some embodiments, the MET-targeted therapy comprises PHA-665752, crizotinib, cabozantinib, or capmatinib (INC280). In some embodiments, the nucleic acid inhibits the expression of the MET fusion nucleic acid molecule or fusion polypeptide encoded by the fusion nucleic acid molecule. In some embodiments, the nucleic acid is a double-stranded RNA (dsRNA), a small interfering RNA (siRNA), or a small hairpin RNA (shRNA). In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

In some embodiments, which may be combined with any of the preceding aspects or embodiments, the fusion nucleic acid molecule is a RAF1 fusion nucleic acid molecule as listed in any of Tables 1-6. In some embodiments, the RAF1 fusion nucleic acid molecule encodes a RAF1 fusion polypeptide. In some embodiments, the encoded RAF1 fusion polypeptide comprises a RAF1 kinase domain, or a fragment of a RAF1 kinase domain having RAF1 kinase activity. In some embodiments, the encoded RAF1 fusion polypeptide has RAF1 kinase activity, optionally wherein the RAF1 kinase activity is constitutive. In some embodiments, the encoded RAF1 fusion polypeptide is oncogenic. In some embodiments, the encoded RAF1 fusion polypeptide promotes cancer cell survival, angiogenesis, cancer cell proliferation, and any combination thereof. In some embodiments, the method further comprises acquiring knowledge of or detecting, in a sample from the individual, the presence of: (a) a mutation in a BRAF gene, optionally wherein the mutation results in a V600E amino acid substitution in an encoded BRAF polypeptide; (b) a mutation in an EGFR gene, optionally wherein the mutation results in a S492R and/or V441G amino acid substitution in an encoded EGFR polypeptide; (c) a wild type KRAS gene, or a mutation in a KRAS gene, optionally wherein the mutation results in a G12C and/or G13D amino acid substitution in an encoded KRAS polypeptide; (d) a mutation in an HRAS gene, optionally wherein the mutation results in a Q61L amino acid substitution in an encoded HRAS polypeptide; (e) a mutation in a MAP2K1 gene, optionally wherein the mutation results in a E102_I103del mutation and/or a K57T amino acid substitution in an encoded MAP2K1 polypeptide; and/or (f) a mutation in an NRAS gene, optionally wherein the mutation results in a Q61K amino acid substitution in an encoded NRAS polypeptide. In some embodiments, the method further comprises acquiring knowledge of or detecting, in a sample from the individual, the presence of: a mutation in a BRAF gene resulting in an V600E amino acid substitution in an encoded BRAF polypeptide; a mutation in an EGFR gene resulting in a S492R and/or V441G amino acid substitution in an encoded EGFR polypeptide; a wild type KRAS gene; a mutation in an HRAS gene resulting in an Q61L amino acid substitution in an encoded HRAS polypeptide; a mutation in a MAP2K1 gene resulting in a E102_I103del mutation and/or a K57T amino acid substitution in an encoded MAP2K1 polypeptide; and a mutation in an NRAS gene resulting in an Q61K amino acid substitution in an encoded NRAS polypeptide, wherein the RAF1 fusion nucleic acid molecule is a RAF1-SYN2 fusion nucleic acid molecule as listed in any of Tables 1 and 3-5. In some embodiments, the cancer is a colorectal cancer. In some embodiments, the cancer was previously treated with 5-FU; folinic acid, 5-FU, and irinotecan (FOLFIRI) in combination with bevacizumab; FOLFIRI in combination with cetuximab; folinic acid, 5-FU, and oxaliplatin (FOLFOX) in combination with bevacizumab; and/or pembrolizumab in combination with regorafenib. In some embodiments, the method further comprises acquiring knowledge of or detecting, in a sample from the individual, the presence of: a mutation in a KRAS gene resulting in a G12C and/or G13D amino acid substitution in an encoded KRAS polypeptide; a mutation in a MAP2K1 gene resulting in a E102_I103del mutation in an encoded MAP2K1 polypeptide; and a mutation in an NRAS gene resulting in an Q61K amino acid substitution in an encoded NRAS polypeptide, wherein the RAF1 fusion nucleic acid molecule is a RAF1-TRAK1 fusion nucleic acid molecule as listed in Tables 2 or 6. In some embodiments, the cancer is a colorectal cancer. In some embodiments, the cancer was previously treated with adagrasib or adagrasib in combination with cetuximab. In some embodiments, the anti-cancer therapy is a RAF1-targeted therapy. In some embodiments, the RAF1-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for a RAF1-rearranged cancer, a RAF1-targeted therapy being tested in a clinical trial, a treatment for RAF1-rearranged cancer being tested in a clinical trial, or any combination thereof. In some embodiments, the RAF1-targeted therapy is a kinase inhibitor. In some embodiments, the RAF1-targeted therapy is a serine/threonine kinase inhibitor. In some embodiments, the RAF1-targeted therapy is a multi-kinase inhibitor or a RAF1-specific inhibitor. In some embodiments, the kinase inhibitor inhibits a kinase activity of a RAF1 polypeptide. In some embodiments, the RAF1-targeted therapy comprises one or more of Sorafenib (BAY49-9006), Binimetinib, Cobimetinib, Regorafenib, Trametinib, or RAF265. In some embodiments, the nucleic acid inhibits the expression of the RAF1 fusion nucleic acid molecule or fusion polypeptide encoded by the fusion nucleic acid molecule. In some embodiments, the nucleic acid is a double-stranded RNA (dsRNA), a small interfering RNA (siRNA), or a small hairpin RNA (shRNA). In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

In some embodiments, which may be combined with any of the preceding aspects or embodiments, the fusion nucleic acid molecule is a RET fusion nucleic acid molecule as listed in any of Tables 1-6. In some embodiments, the RET fusion nucleic acid molecule encodes a RET fusion polypeptide. In some embodiments, the encoded RET fusion polypeptide comprises a RET kinase domain, or a fragment of a RET kinase domain having RET kinase activity. In some embodiments, the encoded RET fusion polypeptide has RET kinase activity, optionally wherein the RET kinase activity is constitutive. In some embodiments, the encoded RET fusion polypeptide is oncogenic. In some embodiments, the encoded RET fusion polypeptide promotes cancer cell survival, angiogenesis, cancer cell proliferation, and any combination thereof. In some embodiments, the method further comprises acquiring knowledge of or detecting, in a sample from the individual, the presence of: (a) a mutation in an EGFR gene; optionally wherein the mutation is a deletion of exon 19 of EGFR or a portion thereof, or a mutation resulting in a T790M amino acid substitution in an encoded EGFR polypeptide, or both; (b) a mutation in a PIK3CA gene; optionally wherein the mutation results in an E542K amino acid substitution in an encoded PIK3CA polypeptide; (c) a mutation in a KRAS gene; optionally wherein the mutation results in a G12C amino acid substitution in an encoded KRAS polypeptide; (d) a mutation in an ESR1 gene; optionally wherein the mutation results in an E380Q amino acid substitution in an encoded ESR1 polypeptide; (e) a mutation in a PTEN gene; optionally wherein the mutation results in a S59* and/or M134I amino acid substitution in an encoded PTEN polypeptide; or any combination of (a)-(e). In some embodiments, the method further comprises acquiring knowledge of or detecting, in a sample from the individual, the presence of a deletion of exon 19 of EGFR or a portion thereof; wherein the RET fusion nucleic acid molecule is a RET-ERC1 fusion nucleic acid molecule as listed in Tables 2 or 6. In some embodiments, the method further comprises acquiring knowledge of or detecting, in a sample from the individual, the presence of a deletion of exon 19 of EGFR or a portion thereof, and an EGFR gene mutation resulting in a T790M amino acid substitution in an encoded EGFR polypeptide; wherein the RET fusion nucleic acid molecule is a RET-NCOA4 fusion nucleic acid molecule as listed in Tables 2 or 6. In some embodiments, the individual was previously treated with osimertinib. In some embodiments, the fusion nucleic acid molecule, and/or the fusion polypeptide encoded by the fusion nucleic acid molecule, confers resistance of the cancer to an EGFR-targeted anti-cancer therapy, optionally wherein the EGFR-targeted anti-cancer therapy is a first-, second-, or third-generation EGFR tyrosine kinase inhibitor. In some embodiments, the fusion nucleic acid molecule, and/or the fusion polypeptide encoded by the fusion nucleic acid molecule, confers resistance of the cancer to an EGFR-targeted anti-cancer therapy, optionally wherein the EGFR-targeted anti-cancer therapy is cetuximab, panitumumab, lapatinib, gefitinib, vandetanib, dacomitinib, icotinib, osimertinib (AZD9291), afatanib, olmutinib, EGF816 (nazartinib), avitinib (AC00010), rociletinib (CO-1686), BMS-690514, YH5448, PF-06747775, ASP8273, PF299804, AP26113, or erlotinib. In some embodiments, the method further comprises acquiring knowledge of or detecting, in a sample from the individual, the presence of: a PIK3CA gene mutation resulting in an E542K amino acid substitution in an encoded PIK3CA polypeptide, an ESR1 gene mutation resulting in a E380Q amino acid substitution in an encoded ESR1 polypeptide, a KRAS gene mutation resulting in a G12C amino acid substitution in an encoded KRAS polypeptide, and a PTEN gene mutation resulting in a S59* and/or M134I amino acid substitution in an encoded PTEN polypeptide; wherein the RET fusion nucleic acid molecule is a RET-BAIAP2L1 fusion nucleic acid molecule as listed in any of Tables 1 and 3-5. In some embodiments, the cancer is a breast cancer. In some embodiments, the fusion nucleic acid molecule, and/or the fusion polypeptide encoded by the fusion nucleic acid molecule, confers resistance of the cancer to a PI3K-targeted therapy. In some embodiments, the method further comprises acquiring knowledge of or detecting, in a sample from the individual, the presence of an EGFR gene mutation resulting in a T790M and/or L858R amino acid substitution in an encoded EGFR polypeptide; wherein the RET fusion nucleic acid molecule is a RET-CCDC6 fusion nucleic acid molecule as listed in Tables 2 or 6. In some embodiments, the anti-cancer therapy is a RET-targeted therapy. In some embodiments, the RET-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for a RET-rearranged cancer, a RET-targeted therapy being tested in a clinical trial, a treatment for RET-rearranged cancer being tested in a clinical trial, or any combination thereof. In some embodiments, the RET-targeted therapy is a kinase inhibitor. In some embodiments, the RET-targeted therapy is a tyrosine kinase inhibitor. In some embodiments, the RET-targeted therapy is a multi-kinase inhibitor or a RET-specific inhibitor. In some embodiments, the kinase inhibitor inhibits a kinase activity of a RET polypeptide. In some embodiments, the RET-targeted therapy comprises one or more of Selpercatinib, Pralsetinib, Alectinib, Cabozantinib, Lenvatinib, Ponatinib, Regorafenib, Sorafenib, Sunitinib, or Vandetanib. In some embodiments, the nucleic acid inhibits the expression of the RET fusion nucleic acid molecule or fusion polypeptide encoded by the fusion nucleic acid molecule. In some embodiments, the nucleic acid is a double-stranded RNA (dsRNA), a small interfering RNA (siRNA), or a small hairpin RNA (shRNA). In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

In some embodiments, which may be combined with any of the preceding aspects or embodiments, the fusion nucleic acid molecule is a ROS1 fusion nucleic acid molecule as listed in any of Tables 1-6. In some embodiments, the ROS1 fusion nucleic acid molecule encodes a ROS1 fusion polypeptide. In some embodiments, the encoded ROS1 fusion polypeptide comprises a ROS1 kinase domain, or a fragment of a ROS1 kinase domain having ROS1 kinase activity. In some embodiments, the encoded ROS1 fusion polypeptide has ROS1 kinase activity, optionally wherein the ROS1 kinase activity is constitutive. In some embodiments, the encoded ROS1 fusion polypeptide is oncogenic. In some embodiments, the encoded ROS1 fusion polypeptide promotes cancer cell survival, angiogenesis, cancer cell proliferation, and any combination thereof. In some embodiments, the method further comprises acquiring knowledge of or detecting, in a sample from the individual, the presence of a PIK3CA gene mutation; optionally wherein the mutation results in an E545K amino acid substitution in an encoded PIK3CA polypeptide. In some embodiments, the ROS1 fusion nucleic acid molecule is a ROS1-GOPC fusion nucleic acid molecule listed Tables 2 or 6, and wherein the sample comprises a PIK3CA gene mutation resulting in an E545K amino acid substitution in an encoded PIK3CA polypeptide. In some embodiments, the fusion nucleic acid molecule, and/or the fusion polypeptide encoded by the fusion nucleic acid molecule, confers resistance of the cancer to a PI3K-targeted therapy. In some embodiments, the anti-cancer therapy is a ROS1-targeted therapy. In some embodiments, the ROS1-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for a ROS1-rearranged cancer, a ROS1-targeted therapy being tested in a clinical trial, a treatment for ROS1-rearranged cancer being tested in a clinical trial, or any combination thereof. In some embodiments, the ROS1-targeted therapy is a kinase inhibitor. In some embodiments, the ROS1-targeted therapy is a tyrosine kinase inhibitor. In some embodiments, the ROS1-targeted therapy is a multi-kinase inhibitor or a ROS1-specific inhibitor. In some embodiments, the kinase inhibitor inhibits a kinase activity of a ROS1 polypeptide. In some embodiments, the ROS1-targeted therapy comprises one or more of crizotinib, lorlatinib, TQ-B3139, repotrectinib (TPX-0005), brigatinib, cabozantinib, ceritinib, or entrectinib. In some embodiments, the nucleic acid inhibits the expression of the ROS1 fusion nucleic acid molecule or fusion polypeptide encoded by the fusion nucleic acid molecule. In some embodiments, the nucleic acid is a double-stranded RNA (dsRNA), a small interfering RNA (siRNA), or a small hairpin RNA (shRNA). In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

In some embodiments, which may be combined with any of the preceding aspects or embodiments, the fusion nucleic acid molecule is an NTRK1 fusion nucleic acid molecule as listed in any of Tables 2 and 6. In some embodiments, the NTRK1 fusion nucleic acid molecule encodes an NTRK1 fusion polypeptide. In some embodiments, the encoded NTRK1 fusion polypeptide comprises an NTRK1 kinase domain, or a fragment of an NTRK1 kinase domain having NTRK1 kinase activity. In some embodiments, the encoded NTRK1 fusion polypeptide has NTRK1 kinase activity, optionally wherein the NTRK1 kinase activity is constitutive. In some embodiments, the encoded NTRK1 fusion polypeptide is oncogenic. In some embodiments, the encoded NTRK1 fusion polypeptide promotes cancer cell survival, angiogenesis, cancer cell proliferation, and any combination thereof. In some embodiments, the anti-cancer therapy is an NTRK1-targeted therapy. In some embodiments, the NTRK1-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for an NTRK1-rearranged cancer, an NTRK1-targeted therapy being tested in a clinical trial, a treatment for NTRK1-rearranged cancer being tested in a clinical trial, or any combination thereof. In some embodiments, the NTRK1-targeted therapy is a kinase inhibitor. In some embodiments, the NTRK1-targeted therapy is a tyrosine kinase inhibitor. In some embodiments, the NTRK1-targeted therapy is a multi-kinase inhibitor or an NTRK1-specific inhibitor. In some embodiments, the kinase inhibitor inhibits a kinase activity of an NTRK1 polypeptide. In some embodiments, the NTRK1-targeted therapy comprises one or more of altiratinib (DCC-2701), AG 879 (Tyrphostin AG 879), an anti-TrK antibody, ARRY 954, AR523, AZ-23, AZ623, a benzotriazole, CEP-2563, danusertib (PHA-739358), entrectinib, DS-6051, GNF 5837, GW 441756, indenopyrrolocarboazole 12a, isothiazole 5n, larotrectinib, lestaurtinib (CEP-701), selitrectinib (LOXO-195), a macrocyclic compound, ONO-5390556, oxindole 3, pegcantratinib (SNA-120), PHA-848125, PLX7486, a pyrazole derivative, a pyrazolo[1; 5a]pyrimidine, a pyridocarbazole, a pyridoquinazolinyl, a pyridotriazole, a pyrrolidinyl thiourea, a pyrrolidinyl urea, a pyrrolo[2; 3-d]pyrimidine, a quinazolinyl, repotrectinib (TPX-0005), Ro 08-2750, a substituted pyrazolo[1; 5a]pyrimidine, sitravatinib (MGCD516), SNA-125, tavilermide, thiazole 20 h, F17752, cabozantinib (XL184), merestinib (LY2801653), belizatinib (TSR-011), dovitinib, ONO-7579, or VMD-928. In some embodiments, the nucleic acid inhibits the expression of the NTRK1 fusion nucleic acid molecule or fusion polypeptide encoded by the fusion nucleic acid molecule. In some embodiments, the nucleic acid is a double-stranded RNA (dsRNA), a small interfering RNA (siRNA), or a small hairpin RNA (shRNA). In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

In some embodiments, which may be combined with any of the preceding aspects or embodiments, the treatment or the one or more treatment options further comprise an additional anti-cancer therapy. In some embodiments, the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy. In some embodiments, the nucleic acid comprises a double-stranded RNA (dsRNA), a small interfering RNA (siRNA), or a small hairpin RNA (shRNA).

In some embodiments, which may be combined with any of the preceding aspects or embodiments, the method further comprises obtaining the sample from the individual. In some embodiments, which may be combined with any of the preceding aspects or embodiments, the sample is obtained from the cancer. In some embodiments, which may be combined with any of the preceding aspects or embodiments, the sample comprises a tissue biopsy sample, a liquid biopsy sample, or a normal control. In some embodiments, the sample is from a tumor biopsy, tumor specimen, or circulating tumor cell. In some embodiments, the sample is a liquid biopsy sample and comprises blood, plasma, cerebrospinal fluid, sputum, stool, urine, or saliva. In some embodiments, the sample comprises cells and/or nucleic acids from the cancer. In some embodiments, the sample comprises mRNA, DNA, circulating tumor DNA (ctDNA), cell-free DNA, or cell-free RNA from the cancer. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof.

In some embodiments, which may be combined with any of the preceding aspects or embodiments, the method comprises acquiring knowledge of or detecting the fusion nucleic acid molecule or the fusion polypeptide encoded by the fusion nucleic acid molecule in a tissue biopsy sample, in a liquid biopsy sample, or in both a tissue biopsy sample and a liquid biopsy sample, from the individual.

In some embodiments, which may be combined with any of the preceding aspects or embodiments, the acquiring knowledge comprises detecting the fusion nucleic acid molecule, or the polypeptide encoded by the fusion nucleic acid molecule, in the sample.

In some embodiments, which may be combined with any of the preceding aspects or embodiments, the detecting comprises detecting a fragment of the fusion nucleic acid molecule comprising a breakpoint or fusion junction.

In some embodiments, which may be combined with any of the preceding aspects or embodiments, the fusion nucleic acid molecule is detected in the sample by one or more of: a nucleic acid hybridization assay, an amplification-based assay, a polymerase chain reaction-restriction fragment length polymorphism (PCR-RFLP) assay, real-time PCR, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), mass-spectrometric genotyping, or sequencing. In some embodiments, the sequencing comprises a massively parallel sequencing (MPS) technique, whole genome sequencing (WGS), whole exome sequencing, targeted sequencing, direct sequencing, or a Sanger sequencing technique; and optionally wherein the massively parallel sequencing (MPS) technique comprises next-generation sequencing (NGS).

In some embodiments, which may be combined with any of the preceding aspects or embodiments, detecting the fusion polypeptide encoded by the fusion nucleic acid molecule comprises detecting a portion of the fusion polypeptide that is encoded by a fragment of the fusion nucleic acid molecule that comprises a breakpoint or a fusion junction.

In some embodiments, which may be combined with any of the preceding aspects or embodiments, the fusion polypeptide is detected in the sample by one or more of: immunoblotting, enzyme linked immunosorbent assay (ELISA), immunohistochemistry, or mass spectrometry.

In some embodiments, which may be combined with any of the preceding aspects or embodiments, the method further comprises selectively enriching for one or more nucleic acids in the sample comprising nucleotide sequences corresponding to the fusion nucleic acid molecule; wherein the selectively enriching produces an enriched sample. In some embodiments, the selectively enriching comprises: (a) combining one or more bait molecules with the sample, thereby hybridizing the one or more bait molecules to one or more nucleic acids in the sample comprising nucleotide sequences corresponding to the fusion nucleic acid molecule and producing nucleic acid hybrids; and (b) isolating the nucleic acid hybrids to produce the enriched sample.

In some embodiments, which may be combined with any of the preceding aspects or embodiments, the one or more bait molecules comprise a capture nucleic acid molecule configured to hybridize to a nucleotide sequence corresponding to the fusion nucleic acid molecule. In some embodiments, the capture nucleic acid molecule comprises between about 10 and about 30 nucleotides, between about 50 and about 1000 nucleotides, between about 100 and about 500 nucleotides, between about 100 and about 300 nucleotides, or between about 100 and about 200 nucleotides. In some embodiments, the one or more bait molecules are conjugated to an affinity reagent or to a detection reagent. In some embodiments, the affinity reagent is an antibody, an antibody fragment, or biotin, or wherein the detection reagent is a fluorescent marker. In some embodiments, the capture nucleic acid molecule comprises a DNA, RNA, or mixed DNA/RNA molecule.

In some embodiments, which may be combined with any of the preceding aspects or embodiments, the selectively enriching comprises amplifying the one or more nucleic acids comprising nucleotide sequences corresponding to the fusion nucleic acid molecule using a polymerase chain reaction (PCR) to produce an enriched sample.

In some embodiments, which may be combined with any of the preceding aspects or embodiments, the method further comprises sequencing the enriched sample.

In some embodiments, which may be combined with any of the preceding aspects or embodiments, the individual is a human.

In another aspect, provided herein is a kit comprising a probe or bait for detecting: (i) an ALK, BRAF, EGFR, ERBB2, FGFR1, FGFR2, FGFR3, MET, RAF1, RET, or ROS1 fusion nucleic acid molecule listed in any of Tables 1 and 3-5, or a fragment thereof comprising a breakpoint or fusion junction; or (ii) an ALK, BRAF, ERBB2, FGFR1, FGFR2, FGFR3, MET, NTRK1, RAF1, RET, or ROS1 fusion nucleic acid molecule listed in Tables 2 or 6, or a fragment thereof comprising a breakpoint or fusion junction, in a sample from an individual having a cancer corresponding to the ALK, BRAF, ERBB2, FGFR1, FGFR2, FGFR3, MET, NTRK1, RAF1, RET, or ROS1 fusion nucleic acid molecule as listed in Tables 2 or 6.

In another aspect, provided herein is a nucleic acid molecule comprising an ALK, BRAF, EGFR, ERBB2, FGFR1, FGFR2, FGFR3, MET, RAF1, RET, or ROS1 fusion nucleic acid molecule listed in any of Tables 1 and 3-5, or a fragment thereof comprising a breakpoint or fusion junction.

In another aspect, provided herein is a vector comprising a nucleic acid molecule described herein.

In another aspect, provided herein is a host cell comprising a vector provided herein.

In another aspect, provided herein is an antibody or antibody fragment that specifically binds to a fusion polypeptide, or to a portion thereof, encoded by an ALK, BRAF, EGFR, ERBB2, FGFR1, FGFR2, FGFR3, MET, RAF1, RET, or ROS1 fusion nucleic acid molecule listed in any of Tables 1 and 3-5, or a fragment thereof comprising a breakpoint or fusion junction.

In another aspect, provided herein is a kit comprising an antibody or antibody fragment for detecting: (i) a fusion polypeptide, or a portion thereof, encoded by an ALK, BRAF, EGFR, ERBB2, FGFR1, FGFR2, FGFR3, MET, RAF1, RET, or ROS1 fusion nucleic acid molecule listed in any of Tables 1 and 3-5, or a fragment thereof comprising a breakpoint or fusion junction; or (ii) a fusion polypeptide, or a portion thereof, encoded by an ALK, BRAF, ERBB2, FGFR1, FGFR2, FGFR3, MET, NTRK1, RAF1, RET, or ROS1 fusion nucleic acid molecule listed in Tables 2 or 6, or a fragment thereof comprising a breakpoint or fusion junction, in a sample from an individual having a cancer corresponding to the ALK, BRAF, ERBB2, FGFR1, FGFR2, FGFR3, MET, NTRK1, RAF1, RET, or ROS1 fusion nucleic acid molecule as listed in Tables 2 or 6.

In another aspect, provided herein is an in vitro use of one or more oligonucleotides for detecting: (i) an ALK, BRAF, EGFR, ERBB2, FGFR1, FGFR2, FGFR3, MET, RAF1, RET, or ROS1 fusion nucleic acid molecule listed in any of Tables 1 and 3-5, or a fragment thereof comprising a breakpoint or fusion junction; or (ii) an ALK, BRAF, ERBB2, FGFR1, FGFR2, FGFR3, MET, NTRK1, RAF1, RET, or ROS1 fusion nucleic acid molecule listed in Tables 2 or 6, or a fragment thereof comprising a breakpoint or fusion junction, in a sample from an individual having a cancer corresponding to the ALK, BRAF, ERBB2, FGFR1, FGFR2, FGFR3, MET, NTRK1, RAF1, RET, or ROS1 fusion nucleic acid molecule as listed in Tables 2 or 6.

In another aspect, provided herein is a kit comprising one or more oligonucleotides for detecting: (i) an ALK, BRAF, EGFR, ERBB2, FGFR1, FGFR2, FGFR3, MET, RAF1, RET, or ROS1 fusion nucleic acid molecule listed in any of Tables 1 and 3-5, or a fragment thereof comprising a breakpoint or fusion junction; or (ii) an ALK, BRAF, ERBB2, FGFR1, FGFR2, FGFR3, MET, NTRK1, RAF1, RET, or ROS1 fusion nucleic acid molecule listed in Tables 2 or 6, or a fragment thereof comprising a breakpoint or fusion junction, in a sample from an individual having a cancer corresponding to the ALK, BRAF, ERBB2, FGFR1, FGFR2, FGFR3, MET, NTRK1, RAF1, RET, or ROS1 fusion nucleic acid molecule as listed in Tables 2 or 6.

In another aspect, provided herein is a system, comprising: a memory configured to store one or more program instructions; and one or more processors configured to execute the one or more program instructions, the one or more program instructions when executed by the one or more processors are configured to: (a) obtain a plurality of sequence reads of one or more nucleic acid molecules, wherein the one or more nucleic acid molecules are derived from a sample obtained from an individual; (b) analyze the plurality of sequence reads for the presence of a fusion nucleic acid molecule, wherein the fusion nucleic acid molecule is an ALK, BRAF, EGFR, ERBB2, FGFR1, FGFR2, FGFR3, MET, RAF1, RET, or ROS1 fusion nucleic acid molecule listed in Table 1; and (c) detect, based on the analyzing, the fusion nucleic acid molecule in the sample.

In another aspect, provided herein is a non-transitory computer readable storage medium comprising one or more programs executable by one or more computer processors for performing a method, comprising: (a) obtaining, using the one or more processors, a plurality of sequence reads of one or more nucleic acid molecules, wherein the one or more nucleic acid molecules are derived from a sample obtained from an individual; (b) analyzing, using the one or more processors, the plurality of sequence reads for the presence of a fusion nucleic acid molecule, wherein the fusion nucleic acid molecule is an ALK, BRAF, EGFR, ERBB2, FGFR1, FGFR2, FGFR3, MET, RAF1, RET, or ROS1 fusion nucleic acid molecule listed in Table 1; and (c) detecting, using the one or more processors and based on the analyzing, the fusion nucleic acid molecule in the sample.

In some embodiments, which may be combined with any of the preceding aspects or embodiments, the sample is from an individual having a cancer. In some embodiments, the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, comprising or resulting from a Breakpoint 1 and/or a Breakpoint 2 corresponding to the ALK, BRAF, EGFR, ERBB2, FGFR1, FGFR2, FGFR3, MET, RAF1, RET, or ROS1 fusion nucleic acid molecule as listed in Table 3. In some embodiments, the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, and the cancer is a carcinoma, a sarcoma, a lymphoma, a leukemia, a myeloma, a germ cell cancer, or a blastoma. In some embodiments, the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, and the cancer is a solid tumor. In some embodiments, the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, and the cancer is a hematologic malignancy. In some embodiments, the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, and the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor. In some embodiments, the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, and the cancer is appendix adenocarcinoma, bladder adenocarcinoma, bladder urothelial (transitional cell) carcinoma, breast cancer not otherwise specified NOS, breast carcinoma NOS, breast invasive ductal carcinoma (IDC), breast invasive lobular carcinoma (ILC), cervix squamous cell carcinoma (SCC), colon adenocarcinoma (CRC), esophagus adenocarcinoma, esophagus carcinoma NOS, esophagus squamous cell carcinoma (SCC), eye intraocular melanoma, gallbladder adenocarcinoma, gastroesophageal junction adenocarcinoma, intra-hepatic cholangiocarcinoma, kidney cancer NOS, liver hepatocellular carcinoma (HCC), lung cancer NOS, lung adenocarcinoma, lung large cell carcinoma, lung non-small cell lung carcinoma (NSCLC) NOS, lung small cell undifferentiated carcinoma, lung squamous cell carcinoma (SCC), ovary cancer NOS, pancreas cancer NOS, pancreas ductal adenocarcinoma, pancreatobiliary carcinoma, prostate cancer NOS, prostate acinar adenocarcinoma, prostate ductal adenocarcinoma, rectum adenocarcinoma (CRC), skin melanoma, small intestine adenocarcinoma, soft tissue sarcoma NOS, stomach adenocarcinoma NOS, unknown primary cancer NOS, unknown primary adenocarcinoma, unknown primary carcinoma (CUP) NOS, unknown primary neuroendocrine tumor, unknown primary squamous cell carcinoma (SCC), or uterus endometrial adenocarcinoma NOS. In some embodiments, (a) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, and the cancer is the cancer corresponding to the ALK, BRAF, EGFR, ERBB2, FGFR1, FGFR2, FGFR3, MET, RAF1, RET, or ROS1 fusion nucleic acid molecule as listed in Table 4; or (b) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, the cancer is the cancer corresponding to the ALK, BRAF, EGFR, ERBB2, FGFR1, FGFR2, FGFR3, MET, RAF1, RET, or ROS1 fusion nucleic acid molecule as listed in Table 5, and the fusion nucleic acid molecule comprises or results from a Breakpoint 1 and/or a Breakpoint 2 corresponding to the ALK, BRAF, EGFR, ERBB2, FGFR1, FGFR2, FGFR3, MET, RAF1, RET, or ROS1 fusion nucleic acid molecule as listed in Table 5.

In another aspect, provided herein is a system, comprising: a memory configured to store one or more program instructions; and one or more processors configured to execute the one or more program instructions, the one or more program instructions when executed by the one or more processors are configured to: (a) obtain a plurality of sequence reads of one or more nucleic acid molecules, wherein the one or more nucleic acid molecules are derived from a sample obtained from an individual having a cancer; (b) analyze the plurality of sequence reads for the presence of a fusion nucleic acid molecule, wherein the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2; and (c) detect, based on the analyzing, the fusion nucleic acid molecule in the sample.

In another aspect, provided herein is a non-transitory computer readable storage medium comprising one or more programs executable by one or more computer processors for performing a method, comprising: (a) obtaining, using the one or more processors, a plurality of sequence reads of one or more nucleic acid molecules, wherein the one or more nucleic acid molecules are derived from a sample obtained from an individual having a cancer; (b) analyzing, using the one or more processors, the plurality of sequence reads for the presence of a fusion nucleic acid molecule, wherein the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2; and (c) detecting, using the one or more processors and based on the analyzing, the fusion nucleic acid molecule in the sample.

In some embodiments, which may be combined with any of the preceding aspects or embodiments, the fusion nucleic acid molecule comprises or results from a Breakpoint 1 and/or a Breakpoint 2 corresponding to the ALK, BRAF, ERBB2, FGFR1, FGFR2, FGFR3, MET, NTRK1, RAF1, RET, or ROS1 fusion nucleic acid molecule as listed in Table 6.

In some embodiments, which may be combined with any of the preceding aspects or embodiments, the plurality of sequence reads is obtained by sequencing; optionally wherein the sequencing comprises use of a massively parallel sequencing (MPS) technique, whole genome sequencing (WGS), whole exome sequencing, targeted sequencing, direct sequencing, or a Sanger sequencing technique; and optionally wherein the massively parallel sequencing technique comprises next generation sequencing (NGS).

In some embodiments, which may be combined with any of the preceding aspects or embodiments, the one or more program instructions when executed by the one or more processors are further configured to generate, based at least in part on the detecting, a genomic profile for the sample.

In some embodiments, which may be combined with any of the preceding aspects or embodiments, the method further comprises generating, based at least in part on the detecting, a genomic profile for the sample.

In some embodiments, which may be combined with any of the preceding aspects or embodiments, the individual is administered a treatment based at least in part on the genomic profile.

In some embodiments, which may be combined with any of the preceding aspects or embodiments, the genomic profile further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, which may be combined with any of the preceding aspects or embodiments, the genomic profile further comprises results from a nucleic acid sequencing-based test.

In another aspect, provided herein is an anti-cancer therapy for use in a method of treating or delaying progression of cancer, wherein the method comprises administering the anti-cancer therapy to an individual, wherein: (a) an ALK, BRAF, EGFR, ERBB2, FGFR1, FGFR2, FGFR3, MET, RAF1, RET, or ROS1 fusion nucleic acid molecule listed in Table 1 or a fragment thereof comprising a breakpoint or fusion junction, or a fusion polypeptide encoded by the fusion nucleic acid molecule, is detected in a sample obtained from the individual; or (b) an ALK, BRAF, ERBB2, FGFR1, FGFR2, FGFR3, MET, NTRK1, RAF1, RET, or ROS1 fusion nucleic acid molecule listed in Table 2 or a fragment thereof comprising a breakpoint or fusion junction, or a fusion polypeptide encoded by the fusion nucleic acid molecule, is detected in a sample obtained from the individual, wherein the individual has a cancer corresponding to the ALK, BRAF, ERBB2, FGFR1, FGFR2, FGFR3, MET, NTRK1, RAF1, RET, or ROS1 fusion nucleic acid molecule as listed in Table 2.

In another aspect, provided herein is an anti-cancer therapy for use in the manufacture of a medicament for treating or delaying progression of cancer, wherein the medicament is to be administered to an individual, wherein: (a) an ALK, BRAF, EGFR, ERBB2, FGFR1, FGFR2, FGFR3, MET, RAF1, RET, or ROS1 fusion nucleic acid molecule listed in Table 1 or a fragment thereof comprising a breakpoint or fusion junction, or a fusion polypeptide encoded by the fusion nucleic acid molecule, is detected in a sample obtained from the individual; or (b) an ALK, BRAF, ERBB2, FGFR1, FGFR2, FGFR3, MET, NTRK1, RAF1, RET, or ROS1 fusion nucleic acid molecule listed in Table 2 or a fragment thereof comprising a breakpoint or fusion junction, or a fusion polypeptide encoded by the fusion nucleic acid molecule, is detected in a sample obtained from the individual, wherein the individual has a cancer corresponding to the ALK, BRAF, ERBB2, FGFR1, FGFR2, FGFR3, MET, NTRK1, RAF1, RET, or ROS1 fusion nucleic acid molecule as listed in Table 2.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIGS. 1A-1B depict the results of hybrid-capture based comprehensive genomic profiling (CGP) assays to detect kinase fusions in circulating tumor DNA (ctDNA) across diverse cancer types, as described in Examples 1-3. FIG. 1A shows the frequency of kinase fusions (percentage, as shown on the x-axis) detected in ctDNA in each of the cancer types indicated on the y-axis. The numbers on each bar show the total number of unique samples with a kinase fusion in that tumor type. FIG. 1B shows a heatmap of kinase fusions detected in the indicated cancer types. Shading in the figure legend on the right (“Fusion Count”) indicates the number of fusions for each of the kinases on the vertical axis identified in the cancer types on the horizontal axis. Cholangio=cholangiocarcinoma; NSCLC=non-small cell lung cancer; CUP=carcinoma of unknown primary; CRC=colorectal cancer; NOS=not otherwise specified.

FIGS. 2A-2B provide an overview of the most frequent kinase fusion partners identified in ctDNA across diverse cancer types, along with identified fusion breakpoint locations. FIG. 2A shows pie charts representing the most frequent fusions identified in each of the indicated cancer types. FIG. 2B shows a lollipop plot of fusion breakpoint locations identified in the indicated kinases.

FIG. 3 shows the frequency of kinase fusions involving the indicated kinases (FGFR2, BRAF, FGFR3, ROS1, RET, and ALK) identified in tissue biopsies (percentage, as shown on the y-axis) and liquid biopsies (percentage, as shown on x-axis) in NSCLC. Arrows indicate statistical significance (p≤0.05).

FIG. 4 provides an overview of an analysis of concordance between kinase fusions identified in ctDNA (liquid biopsy; n=571) and in tissue (tissue biopsy; n=7,599), as described in Example 2.

FIG. 5 shows sensitivity (percent of positive agreement [PPA]) for detecting kinase fusions (y-axis) in cases with both tissue and liquid biopsy results for each of the groups on the x-axis. Of 4,722 tissue-ctDNA matched pairs, 169 pairs harbored a fusion in either the tissue or liquid specimen. PPA for disease and kinase-specific subsets with at least 20 pairs are shown. BBLB1=blood-based liquid biopsy assay #1.

FIG. 6 depicts the impact of ctDNA fraction on concordance of kinase fusions identified in liquid and tissue biopsies. The y-axis shows the estimated ctDNA fraction (as percentage; calculated as described in Example 2) for each of the groups described in the x-axis. “Concordant” refers to cases in which the same kinase fusion was identified in both liquid and tissue biopsies; “Tissue-negative” refers to cases in which a kinase fusion was identified in liquid biopsies only (and not in tissue biopsies); and “Liquid-negative” refers to cases in which a kinase fusion was identified in tissue biopsies only (and not in liquid biopsies). Arrows indicate the median ctDNA fraction.

FIGS. 7A-7B depict sensitivity (percent of positive agreement [PPA]) for kinase fusion detection in cases with both tissue and liquid biopsy CGP results. FIG. 7A shows sensitivity (PPA) for detecting kinase fusions in ctDNA (y-axis) at each estimated ctDNA fraction in the liquid biopsy as indicated on the x-axis in any cancer type (“All pairs”; n=169) or in NSCLC (n=103). The numbers on top of each bar show the number of tissue and liquid biopsy pairs with a fusion identified in the tissue or liquid biopsy. FIG. 7B shows sensitivity (PPA) for detecting kinase fusions in ctDNA (y-axis) at each timeframe between tissue and liquid specimen collection indicated on the x-axis (<1 year or ≥1 year between collection of the tissue and liquid biopsy samples) in any cancer type (“All pairs”; n=106) or in NSCLC (n=59). The numbers on top of each bar show the number of tissue and liquid biopsy pairs with a fusion identified in the tissue or liquid biopsy.

FIG. 8 depicts the impact of time between specimen collection on concordance between kinase fusions identified in liquid and in tissue biopsies. The y-axis shows the number of days between liquid and tissue biopsy specimen collection, calculated as described in Example 2, for each of the groups described in the x-axis. “Concordant” refers to cases in which the same kinase fusion was identified in both liquid and tissue biopsies; “Tissue-negative” refers to cases in which a kinase fusion was identified in liquid biopsies only (and not in tissue biopsies); and “Liquid-negative” refers to cases in which a kinase fusion was identified in tissue biopsies only (and not in liquid biopsies). Arrows indicate the median number of days between specimen collection.

FIG. 9 shows ALK fusions identified in liquid biopsy specimens with a known ALK resistance mutation. The legend and the top of the figure indicates the gene fusion partner (e.g., “EML4” indicates an ALK-EML4 kinase fusion). ALK mutations identified in samples comprising each of the indicated ALK fusions are shown as shaded boxes. The asterisk indicates the presence of an EGFR L858R mutation.

FIG. 10 depicts an exemplary device, in accordance with some embodiments.

FIG. 11 depicts an exemplary system, in accordance with some embodiments.

FIG. 12 depicts a block diagram of an exemplary process for detecting a fusion nucleic acid molecule, in accordance with some embodiments.

### DETAILED DESCRIPTION

The present disclosure relates generally to detecting kinase fusions in cancer, as well as methods of treatment, and uses related thereto.

Kinase fusions are an important class of targetable oncogenic driver variants. The present disclosure describes a study of a real-world dataset comprising high-quality, validated hybrid capture-based next-generation sequencing (NGS) results that characterized the pan-cancer landscape of kinase fusions involving the ALK, BRAF, EGFR, ERBB2, FGFR1/2/3, MET, NTRK1/2/3, PDGFRA/B, RAF1, RET, and ROS1 kinases in circulating tumor DNA (ctDNA) samples and tumor tissue samples. As described herein, Applicants discovered a multitude of kinase fusions spanning a diversity of cancer types, oncogenes, and breakpoints, and unexpectedly found at least 571 kinase fusions in ctDNA samples. See, e.g., Example 1. Advantageously, genomic profiling of ctDNA closely recapitulated the results of tissue-based testing, and the majority of discordances between tissue and ctDNA results were attributed to a combination of biological and/or analytical factors. See, e.g., Example 2. Applicant further discovered that, unexpectedly, analysis of ctDNA, e.g., by liquid biopsy, identified targetable kinase fusions that were associated with acquired resistance to anti-cancer therapies. See, e.g., Example 3. Accordingly, without wishing to be bound by theory, it is thought that the presence of a kinase fusion described herein in a sample, e.g., a liquid biopsy sample comprising ctDNA and/or a tissue sample such as a tumor biopsy, from individuals having cancer may identify cancer patients who are likely to respond to treatment with an anti-cancer therapy such as a targeted anti-cancer therapy, e.g., as described herein.

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### II. Definitions

As used in this specification and the appended claims, the singular forms “a”, “an” and “the” include plural referents unless the context clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

The terms “cancer” and “cancerous” refer to or describe the physiological condition in mammals that is typically characterized by unregulated cell growth. Included in this definition are benign and malignant cancers.

The term “tumor,” as used herein, refers to all neoplastic cell growth and proliferation, whether malignant or benign, and all pre-cancerous and cancerous cells and tissues. The terms “cancer,” “cancerous,” and “tumor” are not mutually exclusive as referred to herein.

“Polynucleotide,” “nucleic acid,” or “nucleic acid molecule” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs.

A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally-occurring nucleotides with an analog, internucleotide modifications such as, for example, those with uncharged linkages (e.g., methyl phosphonates, phosphotriesters, phosphoamidates, carbamates, and the like) and with charged linkages (e.g., phosphorothioates, phosphorodithioates, and the like), those containing pendant moieties, such as, for example, proteins (e.g., nucleases, toxins, antibodies, signal peptides, poly-L-lysine, and the like), those with intercalators (e.g., acridine, psoralen, and the like), those containing chelators (e.g., metals, radioactive metals, boron, oxidative metals, and the like), those containing alkylators, those with modified linkages (e.g., alpha anomeric nucleic acids), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2-O-methyl-, 2-O-allyl-, 2′-fluoro-, or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs, and abasic nucleoside analogs such as methyl riboside. One or more phosphodiester linkages may be replaced by alternative linking groups. These alternative linking groups include, but are not limited to, embodiments wherein phosphate is replaced by P(0)S (“thioate”), P(S)S (“dithioate”), “(0)NR₂ (“amidate”), P(O)R, P(0)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. A polynucleotide can contain one or more different types of modifications as described herein and/or multiple modifications of the same type. The preceding description applies to all polynucleotides referred to herein, including RNA and DNA.

“Oligonucleotide,” as used herein, generally refers to short, single stranded, polynucleotides that are, but not necessarily, less than about 250 nucleotides in length. Oligonucleotides may be synthetic. The terms “oligonucleotide” and “polynucleotide” are not mutually exclusive. The description above for polynucleotides is equally and fully applicable to oligonucleotides.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “isolated” antibody is one which has been identified and separated and/or recovered from a component of its natural environment. Contaminant components of its natural environment are materials which would interfere with research, diagnostic, and/or therapeutic uses for the antibody, and may include enzymes, hormones, and other proteinaceous or nonproteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. An isolated antibody includes the antibody in situ within recombinant cells since at least one component of the antibody's natural environment will not be present. Ordinarily, however, an isolated antibody will be prepared by at least one purification step.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The “light chains” of antibodies (immunoglobulins) from any mammalian species can be assigned to one of two clearly distinct types, called kappa (“κ”) and lambda (“λ”), based on the amino acid sequences of their constant domains.

The term “constant domain” refers to the portion of an immunoglobulin molecule having a more conserved amino acid sequence relative to the other portion of the immunoglobulin, the variable domain, which contains the antigen binding site. The constant domain contains the CH1, CH2, and CH3 domains (collectively, CH) of the heavy chain and the CHL (or CL) domain of the light chain.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

The term “hypervariable region,” “HVR,” or “HV,” as used herein, refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, for example, Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, for example, Hamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B (Kabat numbering) |
| H1 | H31-H35 | H26-H35 | H26-H32 | H30-H35 (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

“Framework” or “FR” residues are those variable domain residues other than the HVR residues as herein defined.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-1 13 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human lgG1 EU antibody.

The terms “full-length antibody,” “intact antibody,” and “whole antibody” are used herein interchangeably to refer to an antibody in its substantially intact form, not antibody fragments as defined below. The terms particularly refer to an antibody with heavy chains that contain an Fc region.

“Antibody fragments” comprise a portion of an intact antibody comprising the antigen-binding region thereof. In some embodiments, the antibody fragment described herein is an antigen-binding fragment. Examples of antibody fragments include Fab, Fab′, F(ab′)₂, and Fv fragments; diabodies; linear antibodies; single-chain antibody molecules; and multispecific antibodies formed from antibody fragments.

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target-binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target-binding sequence can be further altered, for example, to improve affinity for the target, to humanize the target-binding sequence, to improve its production in cell culture, to reduce its immunogenicity in vivo, to create a multispecific antibody, etc., and that an antibody comprising the altered target-binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the invention may be made by a variety of techniques, including, for example, the hybridoma method (e.g., Kohler and Milstein, Nature 256:495-97 (1975); Hongo et al., Hybridoma 14 (3): 253-260 (1995), Harlow et al., Antibodies: A Laboratory Manual (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101 (34): 12467-12472 (2004); and Lee et al., J. Immunol. Methods 284(1-2): 119-132 (2004)), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg et al., Intern. Rev. Immunol. 13: 65-93 (1995)).

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human framework regions (FRs). In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody.

A “humanized form” of an antibody, e.g., a non-human antibody, refers to an antibody that has undergone humanization.

A “blocking” antibody or an “antagonist” antibody is one which inhibits or reduces biological activity of the antigen it binds. For example, blocking antibodies or antagonist antibodies substantially or completely inhibit the biological activity of the antigen.

As used herein, the term “binds”, “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of <1 μM, <100 nM, <10 nM, <1 nM, or <0.1 nM. In certain embodiments, an antibody specifically binds to an epitope on a protein that is conserved among the protein from different species. In another embodiment, specific binding can include, but does not require exclusive binding.

“Percent (%) amino acid sequence identity” with respect to the polypeptide sequences identified herein is defined as the percentage of amino acid residues in a candidate sequence that are identical with the amino acid residues in the polypeptide being compared, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2, or Megalign (DNASTAR) software. Those skilled in the art can determine appropriate parameters for measuring alignment, including any algorithms needed to achieve maximal alignment over the full-length of the sequences being compared.

The term “detection” includes any means of detecting, including direct and indirect detection. The term “biomarker” as used herein (e.g., a “biomarker” such as a kinase fusion or a fusion nucleic acid molecule or polypeptide described herein) refers to an indicator, e.g., predictive, diagnostic, and/or prognostic, which can be detected in a sample. The biomarker may serve as an indicator of a particular subtype of a disease or disorder (e.g., cancer) characterized by certain, molecular, pathological, histological, and/or clinical features (e.g., responsiveness to therapy including a checkpoint inhibitor). In some embodiments, a biomarker is a collection of genes or a collective number of mutations/alterations (e.g., somatic mutations) in a collection of genes. Biomarkers include, but are not limited to, polynucleotides (e.g., DNA and/or RNA), polynucleotide alterations (e.g., polynucleotide copy number alterations, e.g., DNA copy number alterations), polypeptides, polypeptide and polynucleotide modifications (e.g., post-translational modifications), carbohydrates, and/or glycolipid-based molecular markers.

“Amplification,” as used herein generally refers to the process of producing multiple copies of a desired sequence. “Multiple copies” mean at least two copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

The technique of “polymerase chain reaction” or “PCR” as used herein generally refers to a procedure wherein minute amounts of a specific piece of nucleic acid, RNA and/or DNA, are amplified as described, for example, in U.S. Pat. No. 4,683,195. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage, or plasmid sequences, etc. See generally Mullis et al., Cold Spring Harbor Symp. Quant. Biol. 51:263 (1987) and Erlich, ed., PCR Technology (Stockton Press, NY, 1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid which is complementary to a particular nucleic acid.

The term “diagnosis” is used herein to refer to the identification or classification of a molecular or pathological state, disease or condition (e.g., cancer). For example, “diagnosis” may refer to identification of a particular type of cancer. “Diagnosis” may also refer to the classification of a particular subtype of cancer, for instance, by histopathological criteria, or by molecular features (e.g., a subtype characterized by expression of one or a combination of biomarkers (e.g., particular genes or proteins encoded by said genes)).

The term “aiding diagnosis” is used herein to refer to methods that assist in making a clinical determination regarding the presence, or nature, of a particular type of symptom or condition of a disease or disorder (e.g., cancer). For example, a method of aiding diagnosis of a disease or condition (e.g., cancer) can comprise measuring certain somatic mutations in a biological sample from an individual.

The term “sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, plasma, serum, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof. In some instances, the sample is a whole blood sample, a plasma sample, a serum sample, or a combination thereof. In some embodiments, the sample is from a tumor (e.g., a “tumor sample”), such as from a biopsy. In some embodiments, the sample is a formalin-fixed paraffin-embedded (FFPE) sample.

A “tumor cell” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein.

A “reference sample,” “reference cell,” “reference tissue,” “control sample,” “control cell,” or “control tissue,” as used herein, refer to a sample, cell, tissue, standard, or level that is used for comparison purposes.

By “correlate” or “correlating” is meant comparing, in any way, the performance and/or results of a first analysis or protocol with the performance and/or results of a second analysis or protocol. For example, one may use the results of a first analysis or protocol in carrying out a second protocol and/or one may use the results of a first analysis or protocol to determine whether a second analysis or protocol should be performed. With respect to the embodiment of polypeptide analysis or protocol, one may use the results of the polypeptide expression analysis or protocol to determine whether a specific therapeutic regimen should be performed. With respect to the embodiment of polynucleotide analysis or protocol, one may use the results of the polynucleotide expression analysis or protocol to determine whether a specific therapeutic regimen should be performed.

“Individual response” or “response” can be assessed using any endpoint indicating a benefit to the individual, including, without limitation, (1) inhibition, to some extent, of disease progression (e.g., cancer progression), including slowing down or complete arrest; (2) a reduction in tumor size; (3) inhibition (i.e., reduction, slowing down, or complete stopping) of cancer cell infiltration into adjacent peripheral organs and/or tissues; (4) inhibition (i.e. reduction, slowing down, or complete stopping) of metastasis; (5) relief, to some extent, of one or more symptoms associated with the disease or disorder (e.g., cancer); (6) increase or extension in the length of survival, including overall survival and progression free survival; and/or (7) decreased mortality at a given point of time following treatment.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and/or progression-free survival); resulting in an objective response (including a complete response or a partial response); or improving signs or symptoms of cancer.

An “effective amount” refers to an amount of a therapeutic agent to treat or prevent a disease or disorder in a mammal. In the case of cancers, the therapeutically effective amount of the therapeutic agent may reduce the number of cancer cells; reduce the primary tumor size; inhibit (i.e., slow to some extent and in some embodiments stop) cancer cell infiltration into peripheral organs; inhibit (i.e., slow to some extent and in some embodiments stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), response rates (e.g., CR and PR), duration of response, and/or quality of life.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the patient herein is a human.

As used herein, “administering” is meant a method of giving a dosage of an agent or a pharmaceutical composition (e.g., a pharmaceutical composition including the agent) to a subject (e.g., a patient). Administering can be by any suitable means, including parenteral, intrapulmonary, and intranasal, and, if desired for local treatment, intralesional administration. Parenteral infusions include, for example, intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

The term “concurrently” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

The term “package insert” is used to refer to instructions customarily included in commercial packages of therapeutic products, that contain information about the indications, usage, dosage, administration, combination therapy, contraindications, and/or warnings concerning the use of such therapeutic products.

An “article of manufacture” is any manufacture (e.g., a package or container) or kit comprising at least one reagent, e.g., a medicament for treatment of a disease or disorder (e.g., cancer), or a reagent for specifically detecting a biomarker (e.g., a kinase fusion or a fusion nucleic acid molecule or polypeptide described herein) described herein. In certain embodiments, the manufacture or kit is promoted, distributed, or sold as a unit for performing the methods described herein.

The phrase “based on” when used herein means that the information about one or more biomarkers (e.g., a kinase fusion or a fusion nucleic acid molecule or polypeptide described herein) is used to inform a treatment decision, information provided on a package insert, or marketing/promotional guidance, etc.

The terms “allele frequency” and “allele fraction” are used interchangeably herein and refer to the fraction of sequence reads corresponding to a particular allele relative to the total number of sequence reads for a genomic locus. The terms “variant allele frequency” and “variant allele fraction” are used interchangeably herein and refer to the fraction of sequence reads corresponding to a particular variant allele relative to the total number of sequence reads for a genomic locus.

### III. Methods, Systems, and Devices

In certain aspects, provided herein are methods for selecting a treatment for an individual having a cancer; methods for identifying one or more treatment options for an individual having a cancer; methods for predicting survival of an individual having a cancer; methods for treating or delaying progression of cancer; methods for monitoring, evaluating or screening an individual having a cancer; methods for assessing an ALK, BRAF, EGFR, ERBB2, FGFR1, FGFR2, FGFR3, MET, NTRK1, RAF1, RET, or ROS1 fusion nucleic acid molecule or a fusion polypeptide encoded by the fusion nucleic acid molecule in a cancer in an individual; methods for detecting an ALK, BRAF, EGFR, ERBB2, FGFR1, FGFR2, FGFR3, MET, NTRK1, RAF1, RET, or ROS1 fusion nucleic acid molecule or a fusion polypeptide encoded by the fusion nucleic acid molecule; methods for detecting the presence or absence of a cancer in an individual; methods for monitoring progression or recurrence of a cancer in an individual; methods for identifying a candidate treatment for a cancer in an individual in need thereof; methods for identifying an individual having a cancer who may benefit from a treatment comprising an anti-cancer therapy; and methods for predicting survival of an individual having a cancer treated with a treatment comprising an anti-cancer therapy.

In some embodiments, the methods provided herein comprise detecting in a sample from an individual, e.g., an individual having cancer, suspected of having cancer, being treated for cancer, or being tested for cancer, an ALK, BRAF, EGFR, ERBB2, FGFR1, FGFR2, FGFR3, MET, NTRK1, RAF1, RET, or ROS1 fusion nucleic acid molecule, or a fusion polypeptide encoded by the fusion nucleic acid molecule. In some embodiments, detection of the fusion nucleic acid molecule, or the fusion polypeptide encoded by the fusion nucleic acid molecule, in the sample identifies the individual as one who may benefit from a treatment comprising an anti-cancer therapy, e.g., as described herein. In some embodiments, the methods comprise selecting an anti-cancer therapy as a treatment for an individual having cancer, e.g., responsive to detection of an ALK, BRAF, EGFR, ERBB2, FGFR1, FGFR2, FGFR3, MET, NTRK1, RAF1, RET, or ROS1 fusion nucleic acid molecule, or a fusion polypeptide encoded by the fusion nucleic acid molecule, in the sample. In some embodiments, the methods comprise generating a report comprising one or more treatment options identified for an individual based at least in part on detection of an ALK, BRAF, EGFR, ERBB2, FGFR1, FGFR2, FGFR3, MET, NTRK1, RAF1, RET, or ROS1 fusion nucleic acid molecule, or a fusion polypeptide encoded by the fusion nucleic acid molecule, in a sample from the individual. In some embodiments, the one or more treatment options comprise an anti-cancer therapy as described herein. In some embodiments, the methods comprise administering to an individual an effective amount of a treatment that comprises an anti-cancer therapy, e.g., as described herein, responsive to detecting an ALK, BRAF, EGFR, ERBB2, FGFR1, FGFR2, FGFR3, MET, NTRK1, RAF1, RET, or ROS1 fusion nucleic acid molecule, or a fusion polypeptide encoded by the fusion nucleic acid molecule, in a sample from the individual. In some embodiments, responsive to detection of an ALK, BRAF, EGFR, ERBB2, FGFR1, FGFR2, FGFR3, MET, NTRK1, RAF1, RET, or ROS1 fusion nucleic acid molecule, or a fusion polypeptide encoded by the fusion nucleic acid molecule, in a sample from an individual, the individual is predicted to have longer survival when treated with a treatment comprising an anti-cancer therapy, e.g., as described herein, as compared to survival of an individual whose cancer does not comprise the fusion nucleic acid molecule or fusion polypeptide encoded by the fusion nucleic acid molecule. In some embodiments, the methods comprise providing an assessment of an ALK, BRAF, EGFR, ERBB2, FGFR1, FGFR2, FGFR3, MET, NTRK1, RAF1, RET, or ROS1 fusion nucleic acid molecule, or a fusion polypeptide encoded by the fusion nucleic acid molecule, e.g., responsive to detecting the presence or absence of the fusion nucleic acid molecule, or the fusion polypeptide encoded by the fusion nucleic acid molecule, in a sample. In some embodiments, the methods comprise detecting or acquiring knowledge of the presence or absence of a cancer in a sample from the individual. In some embodiments, the methods comprise detecting, in a first sample obtained from an individual at a first time point, the presence or absence of an ALK, BRAF, EGFR, ERBB2, FGFR1, FGFR2, FGFR3, MET, NTRK1, RAF1, RET, or ROS1 fusion nucleic acid molecule, or a fusion polypeptide encoded by the fusion nucleic acid molecule; detecting, in a second sample obtained from the individual at a second time point after the first time point, the presence or absence of an ALK, BRAF, EGFR, ERBB2, FGFR1, FGFR2, FGFR3, MET, NTRK1, RAF1, RET, or ROS1 fusion nucleic acid molecule, or a fusion polypeptide encoded by the fusion nucleic acid molecule; and providing an assessment of cancer progression or cancer recurrence in the individual based, at least in part, on the presence or absence of the fusion nucleic acid molecule, or the fusion polypeptide encoded by the fusion nucleic acid molecule, in the first sample and/or in the second sample. In some embodiments, the methods comprise performing DNA sequencing on a sample obtained from an individual to determine a sequencing mutation profile on a gene, wherein the sequencing mutation profile identifies the presence or absence of an ALK, BRAF, EGFR, ERBB2, FGFR1, FGFR2, FGFR3, MET, NTRK1, RAF1, RET, or ROS1 fusion nucleic acid molecule. In some embodiments, the methods comprise identifying a candidate treatment based, at least in part, on a sequencing mutation profile on a gene. In some embodiments, the candidate treatment comprises an anti-cancer therapy described herein. In some embodiments, the candidate treatment is identified based, at least in part, on the presence of an ALK, BRAF, EGFR, ERBB2, FGFR1, FGFR2, FGFR3, MET, NTRK1, RAF1, RET, or ROS1 fusion nucleic acid molecule as identified in the sequencing mutation profile.

In some embodiments, the methods provided herein comprise acquiring knowledge of an ALK, BRAF, EGFR, ERBB2, FGFR1, FGFR2, FGFR3, MET, NTRK1, RAF1, RET, or ROS1 fusion nucleic acid molecule, or a fusion polypeptide encoded by the fusion nucleic acid molecule, in a sample from an individual, e.g., an individual having cancer, suspected of having cancer, being treated for cancer, or being tested for cancer. In some embodiments, knowledge of the presence of an ALK, BRAF, EGFR, ERBB2, FGFR1, FGFR2, FGFR3, MET, NTRK1, RAF1, RET, or ROS1 fusion nucleic acid molecule, or a fusion polypeptide encoded by the fusion nucleic acid molecule, in a sample from an individual identifies the individual as one who may benefit from a treatment comprising an anti-cancer therapy, e.g., a described herein. In some embodiments, the methods comprise selecting an anti-cancer therapy, e.g., as described herein, as a treatment for an individual having cancer, e.g., responsive to knowledge of the presence of an ALK, BRAF, EGFR, ERBB2, FGFR1, FGFR2, FGFR3, MET, NTRK1, RAF1, RET, or ROS1 fusion nucleic acid molecule, or a fusion polypeptide encoded by the fusion nucleic acid molecule, in a sample from the individual. In some embodiments, the methods comprise generating a report comprising one or more treatment options identified for an individual based at least in part on knowledge of the presence of an ALK, BRAF, EGFR, ERBB2, FGFR1, FGFR2, FGFR3, MET, NTRK1, RAF1, RET, or ROS1 fusion nucleic acid molecule, or a fusion polypeptide encoded by the fusion nucleic acid molecule in sample from the individual. In some embodiments, the one or more treatment options comprise an anti-cancer therapy described herein. In some embodiments, responsive to acquisition of knowledge of the presence of an ALK, BRAF, EGFR, ERBB2, FGFR1, FGFR2, FGFR3, MET, NTRK1, RAF1, RET, or ROS1 fusion nucleic acid molecule, or a fusion polypeptide encoded by the fusion nucleic acid molecule, in a sample from an individual, the individual is classified as a candidate to receive a treatment comprising an anti-cancer therapy, e.g., as described herein. In some embodiments, responsive to acquisition of knowledge of the presence of an ALK, BRAF, EGFR, ERBB2, FGFR1, FGFR2, FGFR3, MET, NTRK1, RAF1, RET, or ROS1 fusion nucleic acid molecule, or a fusion polypeptide encoded by the fusion nucleic acid molecule, in a sample from an individual, the individual is identified as likely to respond to a treatment that comprises an anti-cancer therapy, e.g., as described herein. In some embodiments, responsive to acquisition of knowledge of the presence of an ALK, BRAF, EGFR, ERBB2, FGFR1, FGFR2, FGFR3, MET, NTRK1, RAF1, RET, or ROS1 fusion nucleic acid molecule, or a fusion polypeptide encoded by the fusion nucleic acid molecule, in a sample from an individual, the individual is predicted to have longer survival when treated with a treatment comprising an anti-cancer therapy, e.g., as compared to survival of an individual whose cancer does not comprise the fusion nucleic acid molecule, or the fusion polypeptide encoded by the fusion nucleic acid molecule. In some embodiments, responsive to acquisition of knowledge of the presence of an ALK, BRAF, EGFR, ERBB2, FGFR1, FGFR2, FGFR3, MET, NTRK1, RAF1, RET, or ROS1 fusion nucleic acid molecule, or a fusion polypeptide encoded by the fusion nucleic acid molecule, in a sample from an individual, the individual is predicted to have longer survival when treated with a treatment comprising an anti-cancer therapy, as compared to an individual whose cancer does not exhibit the fusion nucleic acid molecule, or the fusion polypeptide encoded by the fusion nucleic acid molecule. In some embodiments, the methods comprise administering to an individual an effective amount of a treatment that comprises an anti-cancer therapy, e.g., as described herein, responsive to acquiring knowledge of an ALK, BRAF, EGFR, ERBB2, FGFR1, FGFR2, FGFR3, MET, NTRK1, RAF1, RET, or ROS1 fusion nucleic acid molecule, or a fusion polypeptide encoded by the fusion nucleic acid molecule, in a sample from the individual. In some embodiments, responsive to acquiring knowledge of an ALK, BRAF, EGFR, ERBB2, FGFR1, FGFR2, FGFR3, MET, NTRK1, RAF1, RET, or ROS1 fusion nucleic acid molecule, or a fusion polypeptide encoded by the fusion nucleic acid molecule, in a sample from an individual, the individual is predicted to have an improved response to treatment with an anti-cancer therapy as compared to an individual whose cancer does not comprise an ALK, BRAF, EGFR, ERBB2, FGFR1, FGFR2, FGFR3, MET, NTRK1, RAF1, RET, or ROS1 fusion nucleic acid molecule, or a fusion polypeptide encoded by the fusion nucleic acid molecule. In some embodiments, the methods comprise providing an assessment of an ALK, BRAF, EGFR, ERBB2, FGFR1, FGFR2, FGFR3, MET, NTRK1, RAF1, RET, or ROS1 fusion nucleic acid molecule, or a fusion polypeptide encoded by the fusion nucleic acid molecule, e.g., responsive to acquiring knowledge of the presence or absence of the fusion nucleic acid molecule, or the fusion polypeptide encoded by the fusion nucleic acid molecule, in a sample from an individual. In some embodiments, the methods comprise detecting or acquiring knowledge of the presence or absence of a cancer in a sample from an individual.

In other aspects, provided herein are systems. In some embodiments, a system of the disclosure comprises a memory configured to store one or more program instructions; and one or more processors configured to execute the one or more program instructions. In some embodiments, the one or more program instructions when executed by the one or more processors are configured to: (a) obtain a plurality of sequence reads of one or more nucleic acid molecules, wherein the one or more nucleic acid molecules are derived from a sample obtained from an individual; (b) analyze the plurality of sequence reads for the presence of an ALK, BRAF, EGFR, ERBB2, FGFR1, FGFR2, FGFR3, MET, NTRK1, RAF1, RET, or ROS1 fusion nucleic acid molecule; and (c) detect, based on the analyzing, the fusion nucleic acid molecule in the sample. In some embodiments, the sample is from an individual having a cancer, suspected of having cancer, being treated for cancer, or being tested for cancer.

In other aspects, provided herein are non-transitory computer readable storage media. In some embodiments, a non-transitory computer readable storage medium of the disclosure comprises one or more programs executable by one or more computer processors for performing a method. In some embodiments, the method comprises (a) obtaining, using the one or more processors, a plurality of sequence reads of one or more nucleic acid molecules, wherein the one or more nucleic acid molecules are derived from a sample obtained from an individual; (b) analyzing, using the one or more processors, the plurality of sequence reads for the presence of an ALK, BRAF, EGFR, ERBB2, FGFR1, FGFR2, FGFR3, MET, NTRK1, RAF1, RET, or ROS1 fusion nucleic acid molecule; and (c) detecting, using the one or more processors and based on the analyzing, the fusion nucleic acid molecule in the sample. In some embodiments, the sample is from an individual having a cancer, suspected of having cancer, being treated for cancer, or being tested for cancer.

In some embodiments of any of the methods, systems, or non-transitory computer readable storage media provided herein, the fusion nucleic acid molecule is an ALK, BRAF, EGFR, ERBB2, FGFR1, FGFR2, FGFR3, MET, RAF1, RET, or ROS1 fusion nucleic acid molecule listed in Table 1. In some embodiments, the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, comprising or resulting from a Breakpoint 1 and/or a Breakpoint 2 corresponding to the ALK, BRAF, EGFR, ERBB2, FGFR1, FGFR2, FGFR3, MET, RAF1, RET, or ROS1 fusion nucleic acid molecule as listed in Table 3. In some embodiments, the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, and the cancer is a carcinoma, a sarcoma, a lymphoma, a leukemia, a myeloma, a germ cell cancer, or a blastoma. In some embodiments, the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, and the cancer is a solid tumor. In some embodiments, the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, and the cancer is a hematologic malignancy. In some embodiments, the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, and the cancer is a B cell cancer, a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor. In some embodiments, the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, and the cancer is appendix adenocarcinoma, bladder adenocarcinoma, bladder urothelial (transitional cell) carcinoma, breast cancer not otherwise specified (NOS), breast carcinoma NOS, breast invasive ductal carcinoma (IDC), breast invasive lobular carcinoma (ILC), cervix squamous cell carcinoma (SCC), colon adenocarcinoma (CRC), esophagus adenocarcinoma, esophagus carcinoma NOS, esophagus squamous cell carcinoma (SCC), eye intraocular melanoma, gallbladder adenocarcinoma, gastroesophageal junction adenocarcinoma, intra-hepatic cholangiocarcinoma, kidney cancer NOS, liver hepatocellular carcinoma (HCC), lung cancer NOS, lung adenocarcinoma, lung large cell carcinoma, lung non-small cell lung carcinoma (NSCLC) NOS, lung small cell undifferentiated carcinoma, lung squamous cell carcinoma (SCC), ovary cancer NOS, pancreas cancer NOS, pancreas ductal adenocarcinoma, pancreatobiliary carcinoma, prostate cancer NOS, prostate acinar adenocarcinoma, prostate ductal adenocarcinoma, rectum adenocarcinoma (CRC), skin melanoma, small intestine adenocarcinoma, soft tissue sarcoma NOS, stomach adenocarcinoma NOS, unknown primary cancer NOS, unknown primary adenocarcinoma, unknown primary carcinoma (CUP) NOS, unknown primary neuroendocrine tumor, unknown primary squamous cell carcinoma (SCC), or uterus endometrial adenocarcinoma NOS. In some embodiments, the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, and the cancer is the cancer corresponding to the ALK, BRAF, EGFR, ERBB2, FGFR1, FGFR2, FGFR3, MET, RAF1, RET, or ROS1 fusion nucleic acid molecule as listed in Table 4. In some embodiments, the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, the cancer is the cancer corresponding to the ALK, BRAF, EGFR, ERBB2, FGFR1, FGFR2, FGFR3, MET, RAF1, RET, or ROS1 fusion nucleic acid molecule as listed in Table 5, and the fusion nucleic acid molecule comprises or results from a Breakpoint 1 and/or a Breakpoint 2 corresponding to the ALK, BRAF, EGFR, ERBB2, FGFR1, FGFR2, FGFR3, MET, RAF1, RET, or ROS1 fusion nucleic acid molecule as listed in Table 5.

In some embodiments of any of the methods, systems, or non-transitory computer readable storage media provided herein, the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2. In some embodiments, the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2, and the fusion nucleic acid molecule comprises or results from a Breakpoint 1 and/or a Breakpoint 2 corresponding to the ALK, BRAF, ERBB2, FGFR1, FGFR2, FGFR3, MET, NTRK1, RAF1, RET, or ROS1 fusion nucleic acid molecule as listed in Table 6.

### A. Kinase Fusions

Certain aspects of the present disclosure relate to genomic rearrangements involving a gene encoding a kinase, such as an ALK, BRAF, EGFR, ERBB2, FGFR1, FGFR2, FGFR3, MET, NTRK1, RAF1, RET, or ROS1 gene. An ALK, BRAF, EGFR, ERBB2, FGFR1, FGFR2, FGFR3, MET, NTRK1, RAF1, RET, or ROS1 rearrangement of the present disclosure may relate to any chromosomal translocation, fusion, or rearrangement involving the locus of an ALK, BRAF, EGFR, ERBB2, FGFR1, FGFR2, FGFR3, MET, NTRK1, RAF1, RET, or ROS1 gene. In some embodiments, the rearrangements of the disclosure result in a fusion nucleic acid molecule that comprises at least a portion of an ALK, BRAF, EGFR, ERBB2, FGFR1, FGFR2, FGFR3, MET, NTRK1, RAF1, RET, or ROS1 gene fused to at least a portion of another gene. Accordingly, certain aspects of the present disclosure relate to ALK, BRAF, EGFR, ERBB2, FGFR1, FGFR2, FGFR3, MET, NTRK1, RAF1, RET, or ROS1 fusion nucleic acid molecules, as well as to ALK, BRAF, EGFR, ERBB2, FGFR1, FGFR2, FGFR3, MET, NTRK1, RAF1, RET, or ROS1 fusion polypeptides encoded by such fusion nucleic acid molecules.

In some aspects, provided herein are rearrangements involving an ALK gene, as well as ALK fusion nucleic acid molecules and polypeptides.

As used herein “anaplastic lymphoma kinase” or “ALK” refer to a gene encoding an ALK mRNA or polypeptide. The ALK gene encodes the ALK receptor tyrosine kinase protein. ALK is also known as CD246, NBLST3, anaplastic lymphoma receptor tyrosine kinase, and ALK receptor tyrosine kinase. In some embodiments, an ALK gene is a human ALK gene. An exemplary ALK gene is represented by NCBI Gene ID No. 238. An exemplary ALK mRNA sequence is represented by NCBI Ref. Seq. NM_004304, provided below as SEQ ID NO: 1. An exemplary amino acid sequence of an ALK polypeptide is represented by NCBI Ref. Seq. NP_004295.

(SEQ ID NO: 1)
AGATGCGATCCAGCGGCTCTGGGGGCGGCAGCGGTGGTAGCAGCTGGTACCTCCCGCCGCCTCTGTTC
GGAGGGTCGCGGGGCACCGAGGTGCTTTCCGGCCGCCCTCTGGTCGGCCACCCAAAGCCGCGGGCGCT
GATGATGGGTGAGGAGGGGGCGGCAAGATTTCGGGCGCCCCTGCCCTGAACGCCCTCAGCTGCTGCCG
CCGGGGCCGCTCCAGTGCCTGCGAACTCTGAGGAGCCGAGGCGCCGGTGAGAGCAAGGACGCTGCAAA
CTTGCGCAGCGCGGGGGCTGGGATTCACGCCCAGAAGTTCAGCAGGCAGACAGTCCGAAGCCTTCCCG
CAGCGGAGAGATAGCTTGAGGGTGCGCAAGACGGCAGCCTCCGCCCTCGGTTCCCGCCCAGACCGGGC
AGAAGAGCTTGGAGGAGCCAAAAGGAACGCAAAAGGCGGCCAGGACAGCGTGCAGCAGCTGGGAGCCG
CCGTTCTCAGCCTTAAAAGTTGCAGAGATTGGAGGCTGCCCCGAGAGGGGACAGACCCCAGCTCCGAC
TGCGGGGGGCAGGAGAGGACGGTACCCAACTGCCACCTCCCTTCAACCATAGTAGTTCCTCTGTACCG
AGCGCAGCGAGCTACAGACGGGGGCGCGGCACTCGGCGCGGAGAGCGGGAGGCTCAAGGTCCCAGCCA
GTGAGCCCAGTGTGCTTGAGTGTCTCTGGACTCGCCCCTGAGCTTCCAGGTCTGTTTCATTTAGACTC
CTGCTCGCCTCCGTGCAGTTGGGGGAAAGCAAGAGACTTGCGCGCACGCACAGTCCTCTGGAGATCAG
GTGGAAGGAGCCGCTGGGTACCAAGGACTGTTCAGAGCCTCTTCCCATCTCGGGGAGAGCGAAGGGTG
AGGCTGGGCCCGGAGAGCAGTGTAAACGGCCTCCTCCGGCGGGATGGGAGCCATCGGGCTCCTGTGGC
TCCTGCCGCTGCTGCTTTCCACGGCAGCTGTGGGCTCCGGGATGGGGACCGGCCAGCGCGCGGGCTCC
CCAGCTGCGGGGCCGCCGCTGCAGCCCCGGGAGCCACTCAGCTACTCGCGCCTGCAGAGGAAGAGTCT
GGCAGTTGACTTCGTGGTGCCCTCGCTCTTCCGTGTCTACGCCCGGGACCTACTGCTGCCACCATCCT
CCTCGGAGCTGAAGGCTGGCAGGCCCGAGGCCCGCGGCTCGCTAGCTCTGGACTGCGCCCCGCTGCTC
AGGTTGCTGGGGCCGGCGCCGGGGGTCTCCTGGACCGCCGGTTCACCAGCCCCGGCAGAGGCCCGGAC
GCTGTCCAGGGTGCTGAAGGGCGGCTCCGTGCGCAAGCTCCGGCGTGCCAAGCAGTTGGTGCTGGAGC
TGGGCGAGGAGGCGATCTTGGAGGGTTGCGTCGGGCCCCCCGGGGAGGCGGCTGTGGGGCTGCTCCAG
TTCAATCTCAGCGAGCTGTTCAGTTGGTGGATTCGCCAAGGCGAAGGGCGACTGAGGATCCGCCTGAT
GCCCGAGAAGAAGGCGTCGGAAGTGGGCAGAGAGGGAAGGCTGTCCGCGGCAATTCGCGCCTCCCAGC
CCCGCCTTCTCTTCCAGATCTTCGGGACTGGTCATAGCTCCTTGGAATCACCAACAAACATGCCTTCT
CCTTCTCCTGATTATTTTACATGGAATCTCACCTGGATAATGAAAGACTCCTTCCCTTTCCTGTCTCA
TCGCAGCCGATATGGTCTGGAGTGCAGCTTTGACTTCCCCTGTGAGCTGGAGTATTCCCCTCCACTGC
ATGACCTCAGGAACCAGAGCTGGTCCTGGCGCCGCATCCCCTCCGAGGAGGCCTCCCAGATGGACTTG
CTGGATGGGCCTGGGGCAGAGCGTTCTAAGGAGATGCCCAGAGGCTCCTTTCTCCTTCTCAACACCTC
AGCTGACTCCAAGCACACCATCCTGAGTCCGTGGATGAGGAGCAGCAGTGAGCACTGCACACTGGCCG
TCTCGGTGCACAGGCACCTGCAGCCCTCTGGAAGGTACATTGCCCAGCTGCTGCCCCACAACGAGGCT
GCAAGAGAGATCCTCCTGATGCCCACTCCAGGGAAGCATGGTTGGACAGTGCTCCAGGGAAGAATCGG
GCGTCCAGACAACCCATTTCGAGTGGCCCTGGAATACATCTCCAGTGGAAACCGCAGCTTGTCTGCAG
TGGACTTCTTTGCCCTGAAGAACTGCAGTGAAGGAACATCCCCAGGCTCCAAGATGGCCCTGCAGAGC
TCCTTCACTTGTTGGAATGGGACAGTCCTCCAGCTTGGGCAGGCCTGTGACTTCCACCAGGACTGTGC
CCAGGGAGAAGATGAGAGCCAGATGTGCCGGAAACTGCCTGTGGGTTTTTACTGCAACTTTGAAGATG
GCTTCTGTGGCTGGACCCAAGGCACACTGTCACCCCACACTCCTCAATGGCAGGTCAGGACCCTAAAG
GATGCCCGGTTCCAGGACCACCAAGACCATGCTCTATTGCTCAGTACCACTGATGTCCCCGCTTCTGA
AAGTGCTACAGTGACCAGTGCTACGTTTCCTGCACCGATCAAGAGCTCTCCATGTGAGCTCCGAATGT
CCTGGCTCATTCGTGGAGTCTTGAGGGGAAACGTGTCCTTGGTGCTAGTGGAGAACAAAACCGGGAAG
GAGCAAGGCAGGATGGTCTGGCATGTCGCCGCCTATGAAGGCTTGAGCCTGTGGCAGTGGATGGTGTT
GCCTCTCCTCGATGTGTCTGACAGGTTCTGGCTGCAGATGGTCGCATGGTGGGGACAAGGATCCAGAG
CCATCGTGGCTTTTGACAATATCTCCATCAGCCTGGACTGCTACCTCACCATTAGCGGAGAGGACAAG
ATCCTGCAGAATACAGCACCCAAATCAAGAAACCTGTTTGAGAGAAACCCAAACAAGGAGCTGAAACC
CGGGGAAAATTCACCAAGACAGACCCCCATCTTTGACCCTACAGTTCATTGGCTGTTCACCACATGTG
GGGCCAGCGGGCCCCATGGCCCCACCCAGGCACAGTGCAACAACGCCTACCAGAACTCCAACCTGAGC
GTGGAGGTGGGGAGCGAGGGCCCCCTGAAAGGCATCCAGATCTGGAAGGTGCCAGCCACCGACACCTA
CAGCATCTCGGGCTACGGAGCTGCTGGCGGGAAAGGCGGGAAGAACACCATGATGCGGTCCCACGGCG
TGTCTGTGCTGGGCATCTTCAACCTGGAGAAGGATGACATGCTGTACATCCTGGTTGGGCAGCAGGGA
GAGGACGCCTGCCCCAGTACAAACCAGTTAATCCAGAAAGTCTGCATTGGAGAGAACAATGTGATAGA
AGAAGAAATCCGTGTGAACAGAAGCGTGCATGAGTGGGCAGGAGGCGGAGGAGGAGGGGGTGGAGCCA
CCTACGTATTTAAGATGAAGGATGGAGTGCCGGTGCCCCTGATCATTGCAGCCGGAGGTGGTGGCAGG
GCCTACGGGGCCAAGACAGACACGTTCCACCCAGAGAGACTGGAGAATAACTCCTCGGTTCTAGGGCT
AAACGGCAATTCCGGAGCCGCAGGTGGTGGAGGTGGCTGGAATGATAACACTTCCTTGCTCTGGGCCG
GAAAATCTTTGCAGGAGGGTGCCACCGGAGGACATTCCTGCCCCCAGGCCATGAAGAAGTGGGGGTGG
GAGACAAGAGGGGGTTTCGGAGGGGGTGGAGGGGGGTGCTCCTCAGGTGGAGGAGGCGGAGGATATAT
AGGCGGCAATGCAGCCTCAAACAATGACCCCGAAATGGATGGGGAAGATGGGGTTTCCTTCATCAGTC
CACTGGGCATCCTGTACACCCCAGCTTTAAAAGTGATGGAAGGCCACGGGGAAGTGAATATTAAGCAT
TATCTAAACTGCAGTCACTGTGAGGTAGACGAATGTCACATGGACCCTGAAAGCCACAAGGTCATCTG
CTTCTGTGACCACGGGACGGTGCTGGCTGAGGATGGCGTCTCCTGCATTGTGTCACCCACCCCGGAGC
CACACCTGCCACTCTCGCTGATCCTCTCTGTGGTGACCTCTGCCCTCGTGGCCGCCCTGGTCCTGGCT
TTCTCCGGCATCATGATTGTGTACCGCCGGAAGCACCAGGAGCTGCAAGCCATGCAGATGGAGCTGCA
GAGCCCTGAGTACAAGCTGAGCAAGCTCCGCACCTCGACCATCATGACCGACTACAACCCCAACTACT
GCTTTGCTGGCAAGACCTCCTCCATCAGTGACCTGAAGGAGGTGCCGCGGAAAAACATCACCCTCATT
CGGGGTCTGGGCCATGGCGCCTTTGGGGAGGTGTATGAAGGCCAGGTGTCCGGAATGCCCAACGACCC
AAGCCCCCTGCAAGTGGCTGTGAAGACGCTGCCTGAAGTGTGCTCTGAACAGGACGAACTGGATTTCC
TCATGGAAGCCCTGATCATCAGCAAATTCAACCACCAGAACATTGTTCGCTGCATTGGGGTGAGCCTG
CAATCCCTGCCCCGGTTCATCCTGCTGGAGCTCATGGCGGGGGGAGACCTCAAGTCCTTCCTCCGAGA
GACCCGCCCTCGCCCGAGCCAGCCCTCCTCCCTGGCCATGCTGGACCTTCTGCACGTGGCTCGGGACA
TTGCCTGTGGCTGTCAGTATTTGGAGGAAAACCACTTCATCCACCGAGACATTGCTGCCAGAAACTGC
CTCTTGACCTGTCCAGGCCCTGGAAGAGTGGCCAAGATTGGAGACTTCGGGATGGCCCGAGACATCTA
CAGGGCGAGCTACTATAGAAAGGGAGGCTGTGCCATGCTGCCAGTTAAGTGGATGCCCCCAGAGGCCT
TCATGGAAGGAATATTCACTTCTAAAACAGACACATGGTCCTTTGGAGTGCTGCTATGGGAAATCTTT
TCTCTTGGATATATGCCATACCCCAGCAAAAGCAACCAGGAAGTTCTGGAGTTTGTCACCAGTGGAGG
CCGGATGGACCCACCCAAGAACTGCCCTGGGCCTGTATACCGGATAATGACTCAGTGCTGGCAACATC
AGCCTGAAGACAGGCCCAACTTTGCCATCATTTTGGAGAGGATTGAATACTGCACCCAGGACCCGGAT
GTAATCAACACCGCTTTGCCGATAGAATATGGTCCACTTGTGGAAGAGGAAGAGAAAGTGCCTGTGAG
GCCCAAGGACCCTGAGGGGGTTCCTCCTCTCCTGGTCTCTCAACAGGCAAAACGGGAGGAGGAGCGCA
GCCCAGCTGCCCCACCACCTCTGCCTACCACCTCCTCTGGCAAGGCTGCAAAGAAACCCACAGCTGCA
GAGATCTCTGTTCGAGTCCCTAGAGGGCCGGCCGTGGAAGGGGGACACGTGAATATGGCATTCTCTCA
GTCCAACCCTCCTTCGGAGTTGCACAAGGTCCACGGATCCAGAAACAAGCCCACCAGCTTGTGGAACC
CAACGTACGGCTCCTGGTTTACAGAGAAACCCACCAAAAAGAATAATCCTATAGCAAAGAAGGAGCCA
CACGACAGGGGTAACCTGGGGCTGGAGGGAAGCTGTACTGTCCCACCTAACGTTGCAACTGGGAGACT
TCCGGGGGCCTCACTGCTCCTAGAGCCCTCTTCGCTGACTGCCAATATGAAGGAGGTACCTCTGTTCA
GGCTACGTCACTTCCCTTGTGGGAATGTCAATTACGGCTACCAGCAACAGGGCTTGCCCTTAGAAGCC
GCTACTGCCCCTGGAGCTGGTCATTACGAGGATACCATTCTGAAAAGCAAGAATAGCATGAACCAGCC
TGGGCCCTGAGCTCGGTCGCACACTCACTTCTCTTCCTTGGGATCCCTAAGACCGTGGAGGAGAGAGA
GGCAATGGCTCCTTCACAAACCAGAGACCAAATGTCACGTTTTGTTTTGTGCCAACCTATTTTGAAGT
ACCACCAAAAAAGCTGTATTTTGAAAATGCTTTAGAAAGGTTTTGAGCATGGGTTCATCCTATTCTTT
CGAAAGAAGAAAATATCATAAAAATGAGTGATAAATACAAGGCCCAGATGTGGTTGCATAAGGTTTTT
ATGCATGTTTGTTGTATACTTCCTTATGCTTCTTTCAAATTGTGTGTGCTCTGCTTCAATGTAGTCAG
AATTAGCTGCTTCTATGTTTCATAGTTGGGGTCATAGATGTTTCCTTGCCTTGTTGATGTGGACATGA
GCCATTTGAGGGGAGAGGGAACGGAAATAAAGGAGTTATTTGTAATGACTAA

In some aspects, provided herein are rearrangements involving a BRAF gene, as well as BRAF fusion nucleic acid molecules and polypeptides.

As used herein “B-Raf proto-oncogene, serine/threonine kinase” or “BRAF” refer to a gene encoding a BRAF mRNA or polypeptide. The BRAF gene encodes the BRAF serine/threonine kinase protein. BRAF is also known as NS7, B-raf, BRAF1, RAFB1, B-RAF1, and B-Raf proto-oncogene, serine/threonine kinase. In some embodiments, a BRAF gene is a human BRAF gene. An exemplary BRAF gene is represented by NCBI Gene ID No. 673. An exemplary BRAF mRNA sequence is represented by NCBI Ref. Seq. NM_004333, provided below as SEQ ID NO: 2. An exemplary amino acid sequence of a BRAF polypeptide is represented by NCBI Ref. Seq. NP_004324.

(SEQ ID NO: 2)
CTTCCCCCAATCCCCTCAGGCTCGGCTGCGCCCGGGGCCGCGGGCCGGTA
CCTGAGGTGGCCCAGGCGCCCTCCGCCCGCGGCGCCGCCCGGGCCGCTCC
TCCCCGCGCCCCCCGCGCCCCCCGCTCCTCCGCCTCCGCCTCCGCCTCCG
CCTCCCCCAGCTCTCCGCCTCCCTTCCCCCTCCCCGCCCGACAGCGGCCG
CTCGGGCCCCGGCTCTCGGTTATAAGATGGCGGCGCTGAGCGGTGGCGGT
GGTGGCGGCGCGGAGCCGGGCCAGGCTCTGTTCAACGGGGACATGGAGCC
CGAGGCCGGCGCCGGCGCCGGCGCCGCGGCCTCTTCGGCTGCGGACCCTG
CCATTCCGGAGGAGGTGTGGAATATCAAACAAATGATTAAGTTGACACAG
GAACATATAGAGGCCCTATTGGACAAATTTGGTGGGGAGCATAATCCACC
ATCAATATATCTGGAGGCCTATGAAGAATACACCAGCAAGCTAGATGCAC
TCCAACAAAGAGAACAACAGTTATTGGAATCTCTGGGGAACGGAACTGAT
TTTTCTGTTTCTAGCTCTGCATCAATGGATACCGTTACATCTTCTTCCTC
TTCTAGCCTTTCAGTGCTACCTTCATCTCTTTCAGTTTTTCAAAATCCCA
CAGATGTGGCACGGAGCAACCCCAAGTCACCACAAAAACCTATCGTTAGA
GTCTTCCTGCCCAACAAACAGAGGACAGTGGTACCTGCAAGGTGTGGAGT
TACAGTCCGAGACAGTCTAAAGAAAGCACTGATGATGAGAGGTCTAATCC
CAGAGTGCTGTGCTGTTTACAGAATTCAGGATGGAGAGAAGAAACCAATT
GGTTGGGACACTGATATTTCCTGGCTTACTGGAGAAGAATTGCATGTGGA
AGTGTTGGAGAATGTTCCACTTACAACACACAACTTTGTACGAAAAACGT
TTTTCACCTTAGCATTTTGTGACTTTTGTCGAAAGCTGCTTTTCCAGGGT
TTCCGCTGTCAAACATGTGGTTATAAATTTCACCAGCGTTGTAGTACAGA
AGTTCCACTGATGTGTGTTAATTATGACCAACTTGATTTGCTGTTTGTCT
CCAAGTTCTTTGAACACCACCCAATACCACAGGAAGAGGCGTCCTTAGCA
GAGACTGCCCTAACATCTGGATCATCCCCTTCCGCACCCGCCTCGGACTC
TATTGGGCCCCAAATTCTCACCAGTCCGTCTCCTTCAAAATCCATTCCAA
TTCCACAGCCCTTCCGACCAGCAGATGAAGATCATCGAAATCAATTTGGG
CAACGAGACCGATCCTCATCAGCTCCCAATGTGCATATAAACACAATAGA
ACCTGTCAATATTGATGACTTGATTAGAGACCAAGGATTTCGTGGTGATG
GAGGATCAACCACAGGTTTGTCTGCTACCCCCCCTGCCTCATTACCTGGC
TCACTAACTAACGTGAAAGCCTTACAGAAATCTCCAGGACCTCAGCGAGA
AAGGAAGTCATCTTCATCCTCAGAAGACAGGAATCGAATGAAAACACTTG
GTAGACGGGACTCGAGTGATGATTGGGAGATTCCTGATGGGCAGATTACA
GTGGGACAAAGAATTGGATCTGGATCATTTGGAACAGTCTACAAGGGAAA
GTGGCATGGTGATGTGGCAGTGAAAATGTTGAATGTGACAGCACCTACAC
CTCAGCAGTTACAAGCCTTCAAAAATGAAGTAGGAGTACTCAGGAAAACA
CGACATGTGAATATCCTACTCTTCATGGGCTATTCCACAAAGCCACAACT
GGCTATTGTTACCCAGTGGTGTGAGGGCTCCAGCTTGTATCACCATCTCC
ATATCATTGAGACCAAATTTGAGATGATCAAACTTATAGATATTGCACGA
CAGACTGCACAGGGCATGGATTACTTACACGCCAAGTCAATCATCCACAG
AGACCTCAAGAGTAATAATATATTTCTTCATGAAGACCTCACAGTAAAAA
TAGGTGATTTTGGTCTAGCTACAGTGAAATCTCGATGGAGTGGGTCCCAT
CAGTTTGAACAGTTGTCTGGATCCATTTTGTGGATGGCACCAGAAGTCAT
CAGAATGCAAGATAAAAATCCATACAGCTTTCAGTCAGATGTATATGCAT
TTGGAATTGTTCTGTATGAATTGATGACTGGACAGTTACCTTATTCAAAC
ATCAACAACAGGGACCAGATAATTTTTATGGTGGGACGAGGATACCTGTC
TCCAGATCTCAGTAAGGTACGGAGTAACTGTCCAAAAGCCATGAAGAGAT
TAATGGCAGAGTGCCTCAAAAAGAAAAGAGATGAGAGACCACTCTTTCCC
CAAATTCTCGCCTCTATTGAGCTGCTGGCCCGCTCATTGCCAAAAATTCA
CCGCAGTGCATCAGAACCCTCCTTGAATCGGGCTGGTTTCCAAACAGAGG
ATTTTAGTCTATATGCTTGTGCTTCTCCAAAAACACCCATCCAGGCAGGG
GGATATGGTGCGTTTCCTGTCCACTGAAACAAATGAGTGAGAGAGTTCAG
GAGAGTAGCAACAAAAGGAAAATAAATGAACATATGTTTGCTTATATGTT
AAATTGAATAAAATACTCTCTTTTTTTTTAAGGTGAACCAAAGAACACTT
GTGTGGTTAAAGACTAGATATAATTTTTCCCCAAACTAAAATTTATACTT
AACATTGGATTTTTAACATCCAAGGGTTAAAATACATAGACATTGCTAAA
AATTGGCAGAGCCTCTTCTAGAGGCTTTACTTTCTGTTCCGGGTTTGTAT
CATTCACTTGGTTATTTTAAGTAGTAAACTTCAGTTTCTCATGCAACTTT
TGTTGCCAGCTATCACATGTCCACTAGGGACTCCAGAAGAAGACCCTACC
TATGCCTGTGTTTGCAGGTGAGAAGTTGGCAGTCGGTTAGCCTGGGTTAG
ATAAGGCAAACTGAACAGATCTAATTTAGGAAGTCAGTAGAATTTAATAA
TTCTATTATTATTCTTAATAATTTTTCTATAACTATTTCTTTTTATAACA
ATTTGGAAAATGTGGATGTCTTTTATTTCCTTGAAGCAATAAACTAAGTT
TCTTTTTATAAATTTTGAGTGCAGGTGACCAAAAATATTGCTGAGGAGTG
GCACGTTTGACATGAGTAAAATGTCTTAACTTCGGATTTTTAGCGGGAAA
ATGTTATAAATTGGAGTTTCTTTTAAATAGCTTTTTTTAAAATACATTAA
GGATGTCTCGCTCATGTAGAAGTCAAATTTTGTTGCAAACGCATTGCTCC
CTTCACACCCAATCTCTCCCCTGCAAAAAATCTTCACAGAATTCTGTGAG
AACTTTTAGGTGTGTTTTTCTTTGAGATACCTCTGGTTGCCAAACACCAG
GTAATAGATTTTTTAAAGTTGTTATTAGATTATTCTTACCTCTCATGATG
CATATTTTAGCAATCACCTTATCATTGTGTCTCATGTTCTGTCCTCCTTA
TATTCTTTGCCCAGCAAGATTCTACTTATGATGAATGAATGCTCTTCTCC
TTTTTTCATTCAATGGTATGAAGTATTTGTTAGGGTTCTTTAGTACTTAC
ACTTTGTTGTGTAGAAAATGACTGTAATGTGGTGGTCAGTGTATTCTTAC
TGTGATTCAGAGGGAATCAAAAGTAGAAAGCAACAGCACGTGGTCCTATC
AAAGATTTGGCCATCTCTGCTTCACTGTCAGCCTCTTAACTATATCTTCA
CTTACTCAATTTGGTTTTGTCATGATTTTTAAATGTAGCCAATAGATCAA
GGTTCTTCCAGTAAACACATATCTGCATAAATGCCTCCTTGAAGTCAATA
AAGAAGGAAATTGAGAAGACTTTAAATTAATGATAATTTAGTTTTTAAGT
ACCCACAAATAAATTTTTGAAACATTTTCTTTATTTGAATACTTAGATGT
CATCCAGGAAAATCACTCAATAATAATTACGGCAAATCTTTAACCCCTCA
TTTGGGTAGCTTAAGATAAGTAATGCCATTATGAATCAGAATTGATTCAT
GACTTTAGTTAAGAAAATGAAAAGGAACATTTCACGTATTTTTAAAAATG
ATACTAAGGAATAAAGAAGTACAACTATTGGAAAATATCTAAGTATATGA
TTTTTAAATCCTCCAGTGGCATTAAATATATGATTATTAGTAATTGTTAG
ATAGGGTTTTATTCATTCACAAATAGAAGACTAGCAAGCATGTAACTAAC
AAAGTTTTTACAAAATTGACTTTGTGGAATGCTCCAAATGTTTGGCCATT
TTGAGGCACAAGGTCAGGGGTCTCTTTATTGATAGAGCTCCTTCTATAAT
TTCCCAGCATACCTGCCTCACAGTTATCTTCCTTTCATTGTTCACTCTCT
TTTTCTTCTCAATGCCATCCTGCCTAGGCTCCCATCATCTGCATCTGACA
CCTTTCCTTTCTTTCTTTACTAGTCTCCTTTGCGATGGGTGTGGCTAAGC
TCTGTAGAGCCACTCAGAAACTCATTGTTCCATTCTGTAGCCAGTAAAAC
ATGCCTCCAAAGTGTCACAGAGTAATTCTACTCTCTCTTTTAAATTAGGT
CCACCGGAAATGTTAGTGAAAGGACATTAAAAATGTGACAGGTGACATGT
TTAGCTAACATGGATCTGGAGAAATAGGAAGCAGTAGAATTAAATGTTTC
CCTTTCAGGTTTAATTGTATTTGTTCTTGGGTTTTGTTTTATACTGAGTT
TTAAATATATTCTCCAAATAAAAACATTATTTTTTCTAACCATATGTAGA
GTTAATCTCTTTGACTAAGTAATTGAAACAAAAGAACATTTGTTCTTTTG
TGACTGCTTTTTTCCTAAAACCTGAGCCCTCTTTTTTTTTTTTGAAATTA
AAGTTGATTTCCTTTTTTTTTTTTTTTTTTTTTTTTTTTTTTTTTTTTTT
TTTGAGACAGAGTCTCGCTCTGTCGCCCAGGCTGGAGTGCAGTGGCGGGA
TCTCGGCTCACTGCAAGCTCCGCCTCCCGGGTTCACGCCATTCTCCTGCC
TCAGCCTCCCAAGTAGCTGGGACTACAGGCGCCCGCCACTACGCCCGGCT
AATTTTTTGTATTTTTAGTAGAGACGGGGTTTCACCGTTTTAGCCGGGAT
GGTCTCGATCTCCTGACCTCGTGATCCGCCCGCCTCGGCCTCCCAAAGTG
CTGGGATTACAGGCGTGAGCCACCGCGCCCGGCCTTAAAAGTTGATTTCC
TTCTTCAGTAAGGAAACCTTTTTATAAATTTGTTTTGCATTTTAAAAGTT
TTACTAATCAATGATGAGGAAAAAGATTTGTCTTCTTGATTTTAAATAGT
TTCAGGATCACAGGATGTAATCAGATGCTTCCAGTTTATTTATTTTCAGG
TATTACACTAGCCATTTAATCTTTTTTATTTATTTATTTTCTTCCTGCCC
CTCGGATGGCATATACCAGCCATTTAGATACTAAACTCTAATAGTTAAAC
CAATAGTTAAAATTGTCCTCTCTAAAACATTGGCTATTTAATATACCAGC
TTAAATGGCCTTTCTCTCAAGTGAGTCACTCTTAGTTTAAGAAAATTATG
TGCCTTTTTAAAAAATATTATGAAATGGTACTTCATGACAGAAACATTTT
ATCAGTTATAGTCTTATTTGATTGAAAATTGTTGAGCATTTCTGTAAAAC
TTTTTACTTTACTAAATATTTCATCTTTCCTGTGACTGTTTTCTCAAAGA
ATTTAAAAGACTCGATGTGTCTATGCCAGAATGTTTCTCATCCTTTTGAA
ACTGCCTGGGCCAGGCGTAGTGGCTCACGCTGTAATCCCAGCACTTTTGG
AGGCCAAGGTGGGCAGATCGCGTGAGCCCAGGAGTTTGAGACCAGCCTGG
ACAACATGGCGAAACGGTGTCTCTACAGAAAAATTTAAAAATTAGCCAAG
CATAGTGGTGCACAACTGTAGCCCCAGCCACTCGGGAGGCTGACGTGGGA
GGATCCCTTGAACCTGGGGGCGGAGGCTGCTGTGAGCCTTCATCATGCCA
CTGCACTCCAGCCTGGGCAACAAAGCAAAACCCTGTCTCAAAAAAAGAAA
AGAAAAAAAGAAACTGCTTGAAAGTCATGACGAAGAATGTCAGGAGGGGA
CTTATTCTGGCTGCAGTTGACTTTCTCCTTAAATGTCAAGTAGTGATTGA
TTTGGATAAGAAGTAAACTGTTACTTTTCATAACATACTTTAAGGAATTT
ATCAAATTCTATGTATAATGCCCATTAAAATATACTCCATTCTGGAGTAA
AGGGTAAGAGTAATATTTTTAAACTAGTTAATAAAGTCTTTAGCTTTCAC
ATAAACCATGATATTTGAGGTGTCTAAAATCACAGGGTCTTTTTTTTTTT
TTTCAGTCTTCCCAGTTGTTCTCTGCTCTATTCCTAAATAAAGTTAACTT
GAAAATGCA

In some aspects, provided herein are rearrangements involving an EGFR gene, as well as EGFR fusion nucleic acid molecules and polypeptides.

As used herein “epidermal growth factor receptor” or “EGFR” refer to a gene encoding an EGFR mRNA or polypeptide. The EGFR gene encodes the EGFR receptor tyrosine kinase protein. EGFR is also known as ERBB, ERRP, HER1, mENA, ERBB1, PIG61, NISBD2, epidermal growth factor receptor, and EGFR receptor tyrosine kinase. In some embodiments, an EGFR gene is a human EGFR gene. An exemplary EGFR gene is represented by NCBI Gene ID No. 1956. An exemplary EGFR mRNA sequence is represented by NCBI Ref. Seq. NM_005228, provided below as SEQ ID NO: 3. An exemplary amino acid sequence of an EGFR polypeptide is represented by NCBI Ref. Seq. NP_005219.

(SEQ ID NO: 3)
AGACGTCCGGGCAGCCCCCGGCGCAGCGCGGCCGCAGCAGCCTCCGCCCC
CCGCACGGTGTGAGCGCCCGACGCGGCCGAGGCGGCCGGAGTCCCGAGCT
AGCCCCGGCGGCCGCCGCCGCCCAGACCGGACGACAGGCCACCTCGTCGG
CGTCCGCCCGAGTCCCCGCCTCGCCGCCAACGCCACAACCACCGCGCACG
GCCCCCTGACTCCGTCCAGTATTGATCGGGAGAGCCGGAGCGAGCTCTTC
GGGGAGCAGCGATGCGACCCTCCGGGACGGCCGGGGCAGCGCTCCTGGCG
CTGCTGGCTGCGCTCTGCCCGGCGAGTCGGGCTCTGGAGGAAAAGAAAGT
TTGCCAAGGCACGAGTAACAAGCTCACGCAGTTGGGCACTTTTGAAGATC
ATTTTCTCAGCCTCCAGAGGATGTTCAATAACTGTGAGGTGGTCCTTGGG
AATTTGGAAATTACCTATGTGCAGAGGAATTATGATCTTTCCTTCTTAAA
GACCATCCAGGAGGTGGCTGGTTATGTCCTCATTGCCCTCAACACAGTGG
AGCGAATTCCTTTGGAAAACCTGCAGATCATCAGAGGAAATATGTACTAC
GAAAATTCCTATGCCTTAGCAGTCTTATCTAACTATGATGCAAATAAAAC
CGGACTGAAGGAGCTGCCCATGAGAAATTTACAGGAAATCCTGCATGGCG
CCGTGCGGTTCAGCAACAACCCTGCCCTGTGCAACGTGGAGAGCATCCAG
TGGCGGGACATAGTCAGCAGTGACTTTCTCAGCAACATGTCGATGGACTT
CCAGAACCACCTGGGCAGCTGCCAAAAGTGTGATCCAAGCTGTCCCAATG
GGAGCTGCTGGGGTGCAGGAGAGGAGAACTGCCAGAAACTGACCAAAATC
ATCTGTGCCCAGCAGTGCTCCGGGCGCTGCCGTGGCAAGTCCCCCAGTGA
CTGCTGCCACAACCAGTGTGCTGCAGGCTGCACAGGCCCCCGGGAGAGCG
ACTGCCTGGTCTGCCGCAAATTCCGAGACGAAGCCACGTGCAAGGACACC
TGCCCCCCACTCATGCTCTACAACCCCACCACGTACCAGATGGATGTGAA
CCCCGAGGGCAAATACAGCTTTGGTGCCACCTGCGTGAAGAAGTGTCCCC
GTAATTATGTGGTGACAGATCACGGCTCGTGCGTCCGAGCCTGTGGGGCC
GACAGCTATGAGATGGAGGAAGACGGCGTCCGCAAGTGTAAGAAGTGCGA
AGGGCCTTGCCGCAAAGTGTGTAACGGAATAGGTATTGGTGAATTTAAAG
ACTCACTCTCCATAAATGCTACGAATATTAAACACTTCAAAAACTGCACC
TCCATCAGTGGCGATCTCCACATCCTGCCGGTGGCATTTAGGGGTGACTC
CTTCACACATACTCCTCCTCTGGATCCACAGGAACTGGATATTCTGAAAA
CCGTAAAGGAAATCACAGGGTTTTTGCTGATTCAGGCTTGGCCTGAAAAC
AGGACGGACCTCCATGCCTTTGAGAACCTAGAAATCATACGCGGCAGGAC
CAAGCAACATGGTCAGTTTTCTCTTGCAGTCGTCAGCCTGAACATAACAT
CCTTGGGATTACGCTCCCTCAAGGAGATAAGTGATGGAGATGTGATAATT
TCAGGAAACAAAAATTTGTGCTATGCAAATACAATAAACTGGAAAAAACT
GTTTGGGACCTCCGGTCAGAAAACCAAAATTATAAGCAACAGAGGTGAAA
ACAGCTGCAAGGCCACAGGCCAGGTCTGCCATGCCTTGTGCTCCCCCGAG
GGCTGCTGGGGCCCGGAGCCCAGGGACTGCGTCTCTTGCCGGAATGTCAG
CCGAGGCAGGGAATGCGTGGACAAGTGCAACCTTCTGGAGGGTGAGCCAA
GGGAGTTTGTGGAGAACTCTGAGTGCATACAGTGCCACCCAGAGTGCCTG
CCTCAGGCCATGAACATCACCTGCACAGGACGGGGACCAGACAACTGTAT
CCAGTGTGCCCACTACATTGACGGCCCCCACTGCGTCAAGACCTGCCCGG
CAGGAGTCATGGGAGAAAACAACACCCTGGTCTGGAAGTACGCAGACGCC
GGCCATGTGTGCCACCTGTGCCATCCAAACTGCACCTACGGATGCACTGG
GCCAGGTCTTGAAGGCTGTCCAACGAATGGGCCTAAGATCCCGTCCATCG
CCACTGGGATGGTGGGGGCCCTCCTCTTGCTGCTGGTGGTGGCCCTGGGG
ATCGGCCTCTTCATGCGAAGGCGCCACATCGTTCGGAAGCGCACGCTGCG
GAGGCTGCTGCAGGAGAGGGAGCTTGTGGAGCCTCTTACACCCAGTGGAG
AAGCTCCCAACCAAGCTCTCTTGAGGATCTTGAAGGAAACTGAATTCAAA
AAGATCAAAGTGCTGGGCTCCGGTGCGTTCGGCACGGTGTATAAGGGACT
CTGGATCCCAGAAGGTGAGAAAGTTAAAATTCCCGTCGCTATCAAGGAAT
TAAGAGAAGCAACATCTCCGAAAGCCAACAAGGAAATCCTCGATGAAGCC
TACGTGATGGCCAGCGTGGACAACCCCCACGTGTGCCGCCTGCTGGGCAT
CTGCCTCACCTCCACCGTGCAGCTCATCACGCAGCTCATGCCCTTCGGCT
GCCTCCTGGACTATGTCCGGGAACACAAAGACAATATTGGCTCCCAGTAC
CTGCTCAACTGGTGTGTGCAGATCGCAAAGGGCATGAACTACTTGGAGGA
CCGTCGCTTGGTGCACCGCGACCTGGCAGCCAGGAACGTACTGGTGAAAA
CACCGCAGCATGTCAAGATCACAGATTTTGGGCTGGCCAAACTGCTGGGT
GCGGAAGAGAAAGAATACCATGCAGAAGGAGGCAAAGTGCCTATCAAGTG
GATGGCATTGGAATCAATTTTACACAGAATCTATACCCACCAGAGTGATG
TCTGGAGCTACGGGGTGACTGTTTGGGAGTTGATGACCTTTGGATCCAAG
CCATATGACGGAATCCCTGCCAGCGAGATCTCCTCCATCCTGGAGAAAGG
AGAACGCCTCCCTCAGCCACCCATATGTACCATCGATGTCTACATGATCA
TGGTCAAGTGCTGGATGATAGACGCAGATAGTCGCCCAAAGTTCCGTGAG
TTGATCATCGAATTCTCCAAAATGGCCCGAGACCCCCAGCGCTACCTTGT
CATTCAGGGGGATGAAAGAATGCATTTGCCAAGTCCTACAGACTCCAACT
TCTACCGTGCCCTGATGGATGAAGAAGACATGGACGACGTGGTGGATGCC
GACGAGTACCTCATCCCACAGCAGGGCTTCTTCAGCAGCCCCTCCACGTC
ACGGACTCCCCTCCTGAGCTCTCTGAGTGCAACCAGCAACAATTCCACCG
TGGCTTGCATTGATAGAAATGGGCTGCAAAGCTGTCCCATCAAGGAAGAC
AGCTTCTTGCAGCGATACAGCTCAGACCCCACAGGCGCCTTGACTGAGGA
CAGCATAGACGACACCTTCCTCCCAGTGCCTGAATACATAAACCAGTCCG
TTCCCAAAAGGCCCGCTGGCTCTGTGCAGAATCCTGTCTATCACAATCAG
CCTCTGAACCCCGCGCCCAGCAGAGACCCACACTACCAGGACCCCCACAG
CACTGCAGTGGGCAACCCCGAGTATCTCAACACTGTCCAGCCCACCTGTG
TCAACAGCACATTCGACAGCCCTGCCCACTGGGCCCAGAAAGGCAGCCAC
CAAATTAGCCTGGACAACCCTGACTACCAGCAGGACTTCTTTCCCAAGGA
AGCCAAGCCAAATGGCATCTTTAAGGGCTCCACAGCTGAAAATGCAGAAT
ACCTAAGGGTCGCGCCACAAAGCAGTGAATTTATTGGAGCATGACCACGG
AGGATAGTATGAGCCCTAAAAATCCAGACTCTTTCGATACCCAGGACCAA
GCCACAGCAGGTCCTCCATCCCAACAGCCATGCCCGCATTAGCTCTTAGA
CCCACAGACTGGTTTTGCAACGTTTACACCGACTAGCCAGGAAGTACTTC
CACCTCGGGCACATTTTGGGAAGTTGCATTCCTTTGTCTTCAAACTGTGA
AGCATTTACAGAAACGCATCCAGCAAGAATATTGTCCCTTTGAGCAGAAA
TTTATCTTTCAAAGAGGTATATTTGAAAAAAAAAAAAAGTATATGTGAGG
ATTTTTATTGATTGGGGATCTTGGAGTTTTTCATTGTCGCTATTGATTTT
TACTTCAATGGGCTCTTCCAACAAGGAAGAAGCTTGCTGGTAGCACTTGC
TACCCTGAGTTCATCCAGGCCCAACTGTGAGCAAGGAGCACAAGCCACAA
GTCTTCCAGAGGATGCTTGATTCCAGTGGTTCTGCTTCAAGGCTTCCACT
GCAAAACACTAAAGATCCAAGAAGGCCTTCATGGCCCCAGCAGGCCGGAT
CGGTACTGTATCAAGTCATGGCAGGTACAGTAGGATAAGCCACTCTGTCC
CTTCCTGGGCAAAGAAGAAACGGAGGGGATGGAATTCTTCCTTAGACTTA
CTTTTGTAAAAATGTCCCCACGGTACTTACTCCCCACTGATGGACCAGTG
GTTTCCAGTCATGAGCGTTAGACTGACTTGTTTGTCTTCCATTCCATTGT
TTTGAAACTCAGTATGCTGCCCCTGTCTTGCTGTCATGAAATCAGCAAGA
GAGGATGACACATCAAATAATAACTCGGATTCCAGCCCACATTGGATTCA
TCAGCATTTGGACCAATAGCCCACAGCTGAGAATGTGGAATACCTAAGGA
TAGCACCGCTTTTGTTCTCGCAAAAACGTATCTCCTAATTTGAGGCTCAG
ATGAAATGCATCAGGTCCTTTGGGGCATAGATCAGAAGACTACAAAAATG
AAGCTGCTCTGAAATCTCCTTTAGCCATCACCCCAACCCCCCAAAATTAG
TTTGTGTTACTTATGGAAGATAGTTTTCTCCTTTTACTTCACTTCAAAAG
CTTTTTACTCAAAGAGTATATGTTCCCTCCAGGTCAGCTGCCCCCAAACC
CCCTCCTTACGCTTTGTCACACAAAAAGTGTCTCTGCCTTGAGTCATCTA
TTCAAGCACTTACAGCTCTGGCCACAACAGGGCATTTTACAGGTGCGAAT
GACAGTAGCATTATGAGTAGTGTGGAATTCAGGTAGTAAATATGAAACTA
GGGTTTGAAATTGATAATGCTTTCACAACATTTGCAGATGTTTTAGAAGG
AAAAAAGTTCCTTCCTAAAATAATTTCTCTACAATTGGAAGATTGGAAGA
TTCAGCTAGTTAGGAGCCCACCTTTTTTCCTAATCTGTGTGTGCCCTGTA
ACCTGACTGGTTAACAGCAGTCCTTTGTAAACAGTGTTTTAAACTCTCCT
AGTCAATATCCACCCCATCCAATTTATCAAGGAAGAAATGGTTCAGAAAA
TATTTTCAGCCTACAGTTATGTTCAGTCACACACACATACAAAATGTTCC
TTTTGCTTTTAAAGTAATTTTTGACTCCCAGATCAGTCAGAGCCCCTACA
GCATTGTTAAGAAAGTATTTGATTTTTGTCTCAATGAAAATAAAACTATA
TTCATTTCCACTCTATTATGCTCTCAAATACCCCTAAGCATCTATACTAG
CCTGGTATGGGTATGAAAGATACAAAGATAAATAAAACATAGTCCCTGAT
TCTAAGAAATTCACAATTTAGCAAAGGAAATGGACTCATAGATGCTAACC
TTAAAACAACGTGACAAATGCCAGACAGGACCCATCAGCCAGGCACTGTG
AGAGCACAGAGCAGGGAGGTTGGGTCCTGCCTGAGGAGACCTGGAAGGGA
GGCCTCACAGGAGGATGACCAGGTCTCAGTCAGCGGGGAGGTGGAAAGTG
CAGGTGCATCAGGGGCACCCTGACCGAGGAAACAGCTGCCAGAGGCCTCC
ACTGCTAAAGTCCACATAAGGCTGAGGTCAGTCACCCTAAACAACCTGCT
CCCTCTAAGCCAGGGGATGAGCTTGGAGCATCCCACAAGTTCCCTAAAAG
TTGCAGCCCCCAGGGGGATTTTGAGCTATCATCTCTGCACATGCTTAGTG
AGAAGACTACACAACATTTCTAAGAATCTGAGATTTTATATTGTCAGTTA
ACCACTTTCATTATTCATTCACCTCAGGACATGCAGAAATATTTCAGTCA
GAACTGGGAAACAGAAGGACCTACATTCTGCTGTCACTTATGTGTCAAGA
AGCAGATGATCGATGAGGCAGGTCAGTTGTAAGTGAGTCACATTGTAGCA
TTAAATTCTAGTATTTTTGTAGTTTGAAACAGTAACTTAATAAAAGAGCA
AAAGCTATTCTAGCTTTCTTCTTCATATTTTAATTTTCCACCATAAAGTT
TAGTTGCTAAATTCTATTAATTTTAAGATTGTGCTTCCCAAAATAGTTCT
CACTTCATCTGTCCAGGGAGGCACAGTTCTGTCTGGTAGAAGCCGCAAAG
CCCTTAGCCTCTTCACGGATCTGGCGACTGTGATGGGCAGGTCAGGAGAG
GAGCTGCCCAAAGTCCCATGATTTTCACCTAACAGCCCTGATCAGTCAGT
ACTCAAAGCTTGGACTCCATCCCTGAAGGTCTTCCTGATTGATAGCCTGG
CCTTAATACCCTACAGAAAGCCTGTCCATTGGCTGTTTCTTCCTCAGTCA
GTTCCTGGAAGACCTTACCCCATGACCCCAGCTTCAGATGTGGTCTTTGG
AAACAGAGGTCGAAGGAAAGTAAGGAGCTGAGAGCTCACATTCATAGGTG
CCGCCAGCCTTCGTGCATCTTCTTGCATCATCTCTAAGGAGCTCCTCTAA
TTACACCATGCCCGTCACCCCATGAGGGATCAGAGAAGGGATGAGTCTTC
TAAACTCTATATTCGCTGTGAGTCCAGGTTGTAAGGGGGAGCACTGTGGA
TGCATCCTATTGCACTCCAGCTGATGACACCAAAGCTTAGGTGTTTGCTG
AAAGTTCTTGATGTTGTGACTTACCACCCCTGCCTCACAACTGCAGACAT
AAGGGGACTATGGATTGCTTAGCAGGAAAGGCACTGGTTCTCAAGGGCGG
CTGCCCTTGGGAATCTTCTGGTCCCAACCAGAAAGACTGTGGCTTGATTT
TCTCAGGTGCAGCCCAGCCGTAGGGCCTTTTCAGAGCACCCCCTGGTTAT
TGCAACATTCATCAAAGTTTCTAGAACCTCTGGCCTAAAGGAAGGGCCTG
GTGGGATCTACTTGGCACTCGCTGGGGGGCCACCCCCCAGTGCCACTCTC
ACTAGGCCTCTGATTGCACTTGTGTAGGATGAAGCTGGTGGGTGATGGGA
ACTCAGCACCTCCCCTCAGGCAGAAAAGAATCATCTGTGGAGCTTCAAAA
GAAGGGGCCTGGAGTCTCTGCAGACCAATTCAACCCAAATCTCGGGGGCT
CTTTCATGATTCTAATGGGCAACCAGGGTTGAAACCCTTATTTCTAGGGT
CTTCAGTTGTACAAGACTGTGGGTCTGTACCAGAGCCCCCGTCAGAGTAG
AATAAAAGGCTGGGTAGGGTAGAGATTCCCATGTGCAGTGGAGAGAACAA
TCTGCAGTCACTGATAAGCCTGAGACTTGGCTCATTTCAAAAGCGTTCAA
TTCATCCTCACCAGCAGTTCAGCTGGAAAGGGGCAAATACCCCCACCTGA
GCTTTGAAAACGCCCTGGGACCCTCTGCATTCTCTAAGTAAGTTATAGAA
ACCAGTCTCTTCCCTCCTTTGTGAGTGAGCTGCTATTCCACGTAGGCAAC
ACCTGTTGAAATTGCCCTCAATGTCTACTCTGCATTTCTTTCTTGTGATA
AGCACACACTTTTATTGCAACATAATGATCTGCTCACATTTCCTTGCCTG
GGGGCTGTAAAACCTTACAGAACAGAAATCCTTGCCTCTTTCACCAGCCA
CACCTGCCATACCAGGGGTACAGCTTTGTACTATTGAAGACACAGACAGG
ATTTTTAAATGTAAATCTATTTTTGTAACTTTGTTGCGGGATATAGTTCT
CTTTATGTAGCACTGAACTTTGTACAATATATTTTTAGAAACTCATTTTT
CTACTAAAACAAACACAGTTTACTTTAGAGAGACTGCAATAGAATCAAAA
TTTGAAACTGAAATCTTTGTTTAAAAGGGTTAAGTTGAGGCAAGAGGAAA
GCCCTTTCTCTCTCTTATAAAAAGGCACAACCTCATTGGGGAGCTAAGCT
AGGTCATTGTCATGGTGAAGAAGAGAAGCATCGTTTTTATATTTAGGAAA
TTTTAAAAGATGATGGAAAGCACATTTAGCTTGGTCTGAGGCAGGTTCTG
TTGGGGCAGTGTTAATGGAAAGGGCTCACTGTTGTTACTACTAGAAAAAT
CCAGTTGCATGCCATACTCTCATCATCTGCCAGTGTAACCCTGTACATGT
AAGAAAAGCAATAACATAGCACTTTGTTGGTTTATATATATAATGTGACT
TCAATGCAAATTTTATTTTTATATTTACAATTGATATGCATTTACCAGTA
TAAACTAGACATGTCTGGAGAGCCTAATAATGTTCAGCACACTTTGGTTA
GTTCACCAACAGTCTTACCAAGCCTGGGCCCAGCCACCCTAGAGAAGTTA
TTCAGCCCTGGCTGCAGTGACATCACCTGAGGAGCTTTTAAAAGCTTGAA
GCCCAGCTACACCTCAGACCGATTAAACGCAAATCTCTGGGGCTGAAACC
CAAGCATTCGTAGTTTTTAAAGCTCCTGAGGTCATTCCAATGTGCGGCCA
AAGTTGAGAACTACTGGCCTAGGGATTAGCCACAAGGACATGGACTTGGA
GGCAAATTCTGCAGGTGTATGTGATTCTCAGGCCTAGAGAGCTAAGACAC
AAAGACCTCCACATCTGTCGCTGAGAGTCAAGAACCTGAACAGAGTTTCC
ATGAAGGTTCTCCAAGCACTAGAAGGGAGAGTGTCTAAACAATGGTTGAA
AAGCAAAGGAAATATAAAACAGACACCTCTTTCCATTTCCTAAGGTTTCT
CTCTTTATTAAGGGTGGACTAGTAATAAAATATAATATTCTTGCTGCTTA
TGCAGCTGACATTGTTGCCCTCCCTAAAGCAACCAAGTAGCCTTTATTTC
CCACAGTGAAAGAAAACGCTGGCCTATCAGTTACATTACAAAAGGCAGAT
TTCAAGAGGATTGAGTAAGTAGTTGGATGGCTTTCATAAAAACAAGAATT
CAAGAAGAGGATTCATGCTTTAAGAAACATTTGTTATACATTCCTCACAA
ATTATACCTGGGATAAAAACTATGTAGCAGGCAGTGTGTTTTCCTTCCAT
GTCTCTCTGCACTACCTGCAGTGTGTCCTCTGAGGCTGCAAGTCTGTCCT
ATCTGAATTCCCAGCAGAAGCACTAAGAAGCTCCACCCTATCACCTAGCA
GATAAAACTATGGGGAAAACTTAAATCTGTGCATACATTTCTGGATGCAT
TTACTTATCTTTAAAAAAAAAGGAATCCTATGACCTGATTTGGCCACAAA
AATAATCTTGCTGTACAATACAATCTCTTGGAAATTAAGAGATCCTATGG
ATTTGATGACTGGTATTAGAGGTGACAATGTAACCGATTAACAACAGACA
GCAATAACTTCGTTTTAGAAACATTCAAGCAATAGCTTTATAGCTTCAAC
ATATGGTACGTTTTAACCTTGAAAGTTTTGCAATGATGAAAGCAGTATTT
GTACAAATGAAAAGCAGAATTCTCTTTTATATGGTTTATACTGTTGATCA
GAAATGTTGATTGTGCATTGAGTATTAAAAAATTAGATGTATATTATTCA
TTGTTCTTTACTCCTGAGTACCTTATAATAATAATAATGTATTCTTTGTT
AACAA

In some aspects, provided herein are rearrangements involving an ERBB2 gene, as well as ERBB2 fusion nucleic acid molecules and polypeptides.

As used herein “erb-b2 receptor tyrosine kinase 2” or “ERBB2” refer to a gene encoding an ERBB2 mRNA or polypeptide. The ERBB2 gene encodes the ERBB2 receptor tyrosine kinase protein. ERBB2 is also known as NEU, NGL, HER2, TKR1, CD340, HER-2, VSCN2, MLN 19, HER-2/neu, epidermal growth factor, and ERBB2 receptor tyrosine kinase. In some embodiments, an ERBB2 gene is a human ERBB2 gene. An exemplary ERBB2 gene is represented by NCBI Gene ID No. 2064. An exemplary ERBB2 mRNA sequence is represented by NCBI Ref. Seq. NM_004448, provided below as SEQ ID NO: 4. An exemplary amino acid sequence of an ERBB2 polypeptide is represented by NCBI Ref. Seq. NP_004439.

(SEQ ID NO: 4)
CCCCTCCATTGGGACCGGAGAAACCAGGGGAGCCCCCCGGGCAGCCGCGC
GCCCCTTCCCACGGGGCCCTTTACTGCGCCGCGCGCCCGGCCCCCACCCC
TCGCAGCACCCCGCGCCCCGCGCCCTCCCAGCCGGGTCCAGCCGGAGCCA
TGGGGCCGGAGCCGCAGTGAGCACCATGGAGCTGGCGGCCTTGTGCCGCT
GGGGGCTCCTCCTCGCCCTCTTGCCCCCCGGAGCCGCGAGCACCCAAGTG
TGCACCGGCACAGACATGAAGCTGCGGCTCCCTGCCAGTCCCGAGACCCA
CCTGGACATGCTCCGCCACCTCTACCAGGGCTGCCAGGTGGTGCAGGGAA
ACCTGGAACTCACCTACCTGCCCACCAATGCCAGCCTGTCCTTCCTGCAG
GATATCCAGGAGGTGCAGGGCTACGTGCTCATCGCTCACAACCAAGTGAG
GCAGGTCCCACTGCAGAGGCTGCGGATTGTGCGAGGCACCCAGCTCTTTG
AGGACAACTATGCCCTGGCCGTGCTAGACAATGGAGACCCGCTGAACAAT
ACCACCCCTGTCACAGGGGCCTCCCCAGGAGGCCTGCGGGAGCTGCAGCT
TCGAAGCCTCACAGAGATCTTGAAAGGAGGGGTCTTGATCCAGCGGAACC
CCCAGCTCTGCTACCAGGACACGATTTTGTGGAAGGACATCTTCCACAAG
AACAACCAGCTGGCTCTCACACTGATAGACACCAACCGCTCTCGGGCCTG
CCACCCCTGTTCTCCGATGTGTAAGGGCTCCCGCTGCTGGGGAGAGAGTT
CTGAGGATTGTCAGAGCCTGACGCGCACTGTCTGTGCCGGTGGCTGTGCC
CGCTGCAAGGGGCCACTGCCCACTGACTGCTGCCATGAGCAGTGTGCTGC
CGGCTGCACGGGCCCCAAGCACTCTGACTGCCTGGCCTGCCTCCACTTCA
ACCACAGTGGCATCTGTGAGCTGCACTGCCCAGCCCTGGTCACCTACAAC
ACAGACACGTTTGAGTCCATGCCCAATCCCGAGGGCCGGTATACATTCGG
CGCCAGCTGTGTGACTGCCTGTCCCTACAACTACCTTTCTACGGACGTGG
GATCCTGCACCCTCGTCTGCCCCCTGCACAACCAAGAGGTGACAGCAGAG
GATGGAACACAGCGGTGTGAGAAGTGCAGCAAGCCCTGTGCCCGAGTGTG
CTATGGTCTGGGCATGGAGCACTTGCGAGAGGTGAGGGCAGTTACCAGTG
CCAATATCCAGGAGTTTGCTGGCTGCAAGAAGATCTTTGGGAGCCTGGCA
TTTCTGCCGGAGAGCTTTGATGGGGACCCAGCCTCCAACACTGCCCCGCT
CCAGCCAGAGCAGCTCCAAGTGTTTGAGACTCTGGAAGAGATCACAGGTT
ACCTATACATCTCAGCATGGCCGGACAGCCTGCCTGACCTCAGCGTCTTC
CAGAACCTGCAAGTAATCCGGGGACGAATTCTGCACAATGGCGCCTACTC
GCTGACCCTGCAAGGGCTGGGCATCAGCTGGCTGGGGCTGCGCTCACTGA
GGGAACTGGGCAGTGGACTGGCCCTCATCCACCATAACACCCACCTCTGC
TTCGTGCACACGGTGCCCTGGGACCAGCTCTTTCGGAACCCGCACCAAGC
TCTGCTCCACACTGCCAACCGGCCAGAGGACGAGTGTGTGGGCGAGGGCC
TGGCCTGCCACCAGCTGTGCGCCCGAGGGCACTGCTGGGGTCCAGGGCCC
ACCCAGTGTGTCAACTGCAGCCAGTTCCTTCGGGGCCAGGAGTGCGTGGA
GGAATGCCGAGTACTGCAGGGGCTCCCCAGGGAGTATGTGAATGCCAGGC
ACTGTTTGCCGTGCCACCCTGAGTGTCAGCCCCAGAATGGCTCAGTGACC
TGTTTTGGACCGGAGGCTGACCAGTGTGTGGCCTGTGCCCACTATAAGGA
CCCTCCCTTCTGCGTGGCCCGCTGCCCCAGCGGTGTGAAACCTGACCTCT
CCTACATGCCCATCTGGAAGTTTCCAGATGAGGAGGGCGCATGCCAGCCT
TGCCCCATCAACTGCACCCACTCCTGTGTGGACCTGGATGACAAGGGCTG
CCCCGCCGAGCAGAGAGCCAGCCCTCTGACGTCCATCATCTCTGCGGTGG
TTGGCATTCTGCTGGTCGTGGTCTTGGGGGTGGTCTTTGGGATCCTCATC
AAGCGACGGCAGCAGAAGATCCGGAAGTACACGATGCGGAGACTGCTGCA
GGAAACGGAGCTGGTGGAGCCGCTGACACCTAGCGGAGCGATGCCCAACC
AGGCGCAGATGCGGATCCTGAAAGAGACGGAGCTGAGGAAGGTGAAGGTG
CTTGGATCTGGCGCTTTTGGCACAGTCTACAAGGGCATCTGGATCCCTGA
TGGGGAGAATGTGAAAATTCCAGTGGCCATCAAAGTGTTGAGGGAAAACA
CATCCCCCAAAGCCAACAAAGAAATCTTAGACGAAGCATACGTGATGGCT
GGTGTGGGCTCCCCATATGTCTCCCGCCTTCTGGGCATCTGCCTGACATC
CACGGTGCAGCTGGTGACACAGCTTATGCCCTATGGCTGCCTCTTAGACC
ATGTCCGGGAAAACCGCGGACGCCTGGGCTCCCAGGACCTGCTGAACTGG
TGTATGCAGATTGCCAAGGGGATGAGCTACCTGGAGGATGTGCGGCTCGT
ACACAGGGACTTGGCCGCTCGGAACGTGCTGGTCAAGAGTCCCAACCATG
TCAAAATTACAGACTTCGGGCTGGCTCGGCTGCTGGACATTGACGAGACA
GAGTACCATGCAGATGGGGGCAAGGTGCCCATCAAGTGGATGGCGCTGGA
GTCCATTCTCCGCCGGCGGTTCACCCACCAGAGTGATGTGTGGAGTTATG
GTGTGACTGTGTGGGAGCTGATGACTTTTGGGGCCAAACCTTACGATGGG
ATCCCAGCCCGGGAGATCCCTGACCTGCTGGAAAAGGGGGAGCGGCTGCC
CCAGCCCCCCATCTGCACCATTGATGTCTACATGATCATGGTCAAATGTT
GGATGATTGACTCTGAATGTCGGCCAAGATTCCGGGAGTTGGTGTCTGAA
TTCTCCCGCATGGCCAGGGACCCCCAGCGCTTTGTGGTCATCCAGAATGA
GGACTTGGGCCCAGCCAGTCCCTTGGACAGCACCTTCTACCGCTCACTGC
TGGAGGACGATGACATGGGGGACCTGGTGGATGCTGAGGAGTATCTGGTA
CCCCAGCAGGGCTTCTTCTGTCCAGACCCTGCCCCGGGCGCTGGGGGCAT
GGTCCACCACAGGCACCGCAGCTCATCTACCAGGAGTGGCGGTGGGGACC
TGACACTAGGGCTGGAGCCCTCTGAAGAGGAGGCCCCCAGGTCTCCACTG
GCACCCTCCGAAGGGGCTGGCTCCGATGTATTTGATGGTGACCTGGGAAT
GGGGGCAGCCAAGGGGCTGCAAAGCCTCCCCACACATGACCCCAGCCCTC
TACAGCGGTACAGTGAGGACCCCACAGTACCCCTGCCCTCTGAGACTGAT
GGCTACGTTGCCCCCCTGACCTGCAGCCCCCAGCCTGAATATGTGAACCA
GCCAGATGTTCGGCCCCAGCCCCCTTCGCCCCGAGAGGGCCCTCTGCCTG
CTGCCCGACCTGCTGGTGCCACTCTGGAAAGGCCCAAGACTCTCTCCCCA
GGGAAGAATGGGGTCGTCAAAGACGTTTTTGCCTTTGGGGGTGCCGTGGA
GAACCCCGAGTACTTGACACCCCAGGGAGGAGCTGCCCCTCAGCCCCACC
CTCCTCCTGCCTTCAGCCCAGCCTTCGACAACCTCTATTACTGGGACCAG
GACCCACCAGAGCGGGGGGCTCCACCCAGCACCTTCAAAGGGACACCTAC
GGCAGAGAACCCAGAGTACCTGGGTCTGGACGTGCCAGTGTGAACCAGAA
GGCCAAGTCCGCAGAAGCCCTGATGTGTCCTCAGGGAGCAGGGAAGGCCT
GACTTCTGCTGGCATCAAGAGGTGGGAGGGCCCTCCGACCACTTCCAGGG
GAACCTGCCATGCCAGGAACCTGTCCTAAGGAACCTTCCTTCCTGCTTGA
GTTCCCAGATGGCTGGAAGGGGTCCAGCCTCGTTGGAAGAGGAACAGCAC
TGGGGAGTCTTTGTGGATTCTGAGGCCCTGCCCAATGAGACTCTAGGGTC
CAGTGGATGCCACAGCCCAGCTTGGCCCTTTCCTTCCAGATCCTGGGTAC
TGAAAGCCTTAGGGAAGCTGGCCTGAGAGGGGAAGCGGCCCTAAGGGAGT
GTCTAAGAACAAAAGCGACCCATTCAGAGACTGTCCCTGAAACCTAGTAC
TGCCCCCCATGAGGAAGGAACAGCAATGGTGTCAGTATCCAGGCTTTGTA
CAGAGTGCTTTTCTGTTTAGTTTTTACTTTTTTTGTTTTGTTTTTTTAAA
GATGAAATAAAGACCCAGGGGGAGAATGGGTGTTGTATGGGGAGGCAAGT
GTGGGGGGTCCTTCTCCACACCCACTTTGTCCATTTGCAAATATATTTTG
GAAAACA

In some aspects, provided herein are rearrangements involving an FGFR1 gene, as well as FGFR1 fusion nucleic acid molecules and polypeptides.

As used herein “Fibroblast growth factor receptor 1” or “FGFR1” refer to a gene encoding an FGFR1 mRNA or polypeptide. The FGFR1 gene encodes the FGFR1 receptor tyrosine kinase protein. FGFR1 is also known as CEK, FLG, HH2, OGD, ECCL, FLT2, KAL2, BFGFR, CD331, FGFBR, FLT-2, HBGFR, N-SAM, FGFR-1, HRTFDS, bFGF-R-1, Fibroblast growth factor receptor 1, and FGFR1 receptor tyrosine kinase. In some embodiments, an FGFR1 gene is a human FGFR1 gene. An exemplary FGFR1 gene is represented by NCBI Gene ID No. 2260. An exemplary FGFR1 mRNA sequence is represented by NCBI Ref. Seq. NM_015850, provided below as SEQ ID NO: 5. An exemplary amino acid sequence of an FGFR1 polypeptide is represented by NCBI Ref. Seq. NP_056934.

(SEQ ID NO: 5)
AGATGCAGGGGCGCAAACGCCAAAGGAGACCAGGCTGTAGGAAGAGAAGG
GCAGAGCGCCGGACAGCTCGGCCCGCTCCCCGTCCTTTGGGGCCGCGGCT
GGGGAACTACAAGGCCCAGCAGGCAGCTGCAGGGGGCGGAGGCGGAGGAG
GGACCAGCGCGGGTGGGAGTGAGAGAGCGAGCCCTCGCGCCCCGCCGGCG
CATAGCGCTCGGAGCGCTCTTGCGGCCACAGGCGCGGCGTCCTCGGCGGC
GGGCGGCAGCTAGCGGGAGCCGGGACGCCGGTGCAGCCGCAGCGCGCGGA
GGAACCCGGGTGTGCCGGGAGCTGGGCGGCCACGTCCGGACGGGACCGAG
ACCCCTCGTAGCGCATTGCGGCGACCTCGCCTTCCCCGGCCGCGAGCGCG
CCGCTGCTTGAAAAGCCGCGGAACCCAAGGACTTTTCTCCGGTCCGAGCT
CGGGGCGCCCCGCAGGGCGCACGGTACCCGTGCTGCAGTCGGGCACGCCG
CGGCGCCGGGGCCTCCGCAGGGCGATGGAGCCCGGTCTGCAAGGAAAGTG
AGGCGCCGCCGCTGCGTTCTGGAGGAGGGGGGCACAAGGTCTGGAGACCC
CGGGTGGCGGACGGGAGCCCTCCCCCCGCCCCGCCTCCGGGGCACCAGCT
CCGGCTCCATTGTTCCCGCCCGGGCTGGAGGCGCCGAGCACCGAGCGCCG
CCGGGAGTCGAGCGCCGGCCGCGGAGCTCTTGCGACCCCGCCAGGACCCG
AACAGAGCCCGGGGGCGGCGGGCCGGAGCCGGGGACGCGGGCACACGCCC
GCTCGCACAAGCCACGGCGGACTCTCCCGAGGCGGAACCTCCACGCCGAG
CGAGGGTCAGTTTGAAAAGGAGGATCGAGCTCACTGTGGAGTATCCATGG
AGATGTGGAGCCTTGTCACCAACCTCTAACTGCAGAACTGGGATGTGGAG
CTGGAAGTGCCTCCTCTTCTGGGCTGTGCTGGTCACAGCCACACTCTGCA
CCGCTAGGCCGTCCCCGACCTTGCCTGAACAAGCCCAGCCCTGGGGAGCC
CCTGTGGAAGTGGAGTCCTTCCTGGTCCACCCCGGTGACCTGCTGCAGCT
TCGCTGTCGGCTGCGGGACGATGTGCAGAGCATCAACTGGCTGCGGGACG
GGGTGCAGCTGGCGGAAAGCAACCGCACCCGCATCACAGGGGAGGAGGTG
GAGGTGCAGGACTCCGTGCCCGCAGACTCCGGCCTCTATGCTTGCGTAAC
CAGCAGCCCCTCGGGCAGTGACACCACCTACTTCTCCGTCAATGTTTCAG
ATGCTCTCCCCTCCTCGGAGGATGATGATGATGATGATGACTCCTCTTCA
GAGGAGAAAGAAACAGATAACACCAAACCAAACCCCGTAGCTCCATATTG
GACATCCCCAGAAAAGATGGAAAAGAAATTGCATGCAGTGCCGGCTGCCA
AGACAGTGAAGTTCAAATGCCCTTCCAGTGGGACCCCAAACCCCACACTG
CGCTGGTTGAAAAATGGCAAAGAATTCAAACCTGACCACAGAATTGGAGG
CTACAAGGTCCGTTATGCCACCTGGAGCATCATAATGGACTCTGTGGTGC
CCTCTGACAAGGGCAACTACACCTGCATTGTGGAGAATGAGTACGGCAGC
ATCAACCACACATACCAGCTGGATGTCGTGGAGCGGTCCCCTCACCGGCC
CATCCTGCAAGCAGGGTTGCCCGCCAACAAAACAGTGGCCCTGGGTAGCA
ACGTGGAGTTCATGTGTAAGGTGTACAGTGACCCGCAGCCGCACATCCAG
TGGCTAAAGCACATCGAGGTGAATGGGAGCAAGATTGGCCCAGACAACCT
GCCTTATGTCCAGATCTTGAAGACTGCTGGAGTTAATACCACCGACAAAG
AGATGGAGGTGCTTCACTTAAGAAATGTCTCCTTTGAGGACGCAGGGGAG
TATACGTGCTTGGCGGGTAACTCTATCGGACTCTCCCATCACTCTGCATG
GTTGACCGTTCTGGAAGCCCTGGAAGAGAGGCCGGCAGTGATGACCTCGC
CCCTGTACCTGGAGATCATCATCTATTGCACAGGGGCCTTCCTCATCTCC
TGCATGGTGGGGTCGGTCATCGTCTACAAGATGAAGAGTGGTACCAAGAA
GAGTGACTTCCACAGCCAGATGGCTGTGCACAAGCTGGCCAAGAGCATCC
CTCTGCGCAGACAGGTAACAGTGTCTGCTGACTCCAGTGCATCCATGAAC
TCTGGGGTTCTTCTGGTTCGGCCATCACGGCTCTCCTCCAGTGGGACTCC
CATGCTAGCAGGGGTCTCTGAGTATGAGCTTCCCGAAGACCCTCGCTGGG
AGCTGCCTCGGGACAGACTGGTCTTAGGCAAACCCCTGGGAGAGGGCTGC
TTTGGGCAGGTGGTGTTGGCAGAGGCTATCGGGCTGGACAAGGACAAACC
CAACCGTGTGACCAAAGTGGCTGTGAAGATGTTGAAGTCGGACGCAACAG
AGAAAGACTTGTCAGACCTGATCTCAGAAATGGAGATGATGAAGATGATC
GGGAAGCATAAGAATATCATCAACCTGCTGGGGGCCTGCACGCAGGATGG
TCCCTTGTATGTCATCGTGGAGTATGCCTCCAAGGGCAACCTGCGGGAGT
ACCTGCAGGCCCGGAGGCCCCCAGGGCTGGAATACTGCTACAACCCCAGC
CACAACCCAGAGGAGCAGCTCTCCTCCAAGGACCTGGTGTCCTGCGCCTA
CCAGGTGGCCCGAGGCATGGAGTATCTGGCCTCCAAGAAGTGCATACACC
GAGACCTGGCAGCCAGGAATGTCCTGGTGACAGAGGACAATGTGATGAAG
ATAGCAGACTTTGGCCTCGCACGGGACATTCACCACATCGACTACTATAA
AAAGACAACCAACGGCCGACTGCCTGTGAAGTGGATGGCACCCGAGGCAT
TATTTGACCGGATCTACACCCACCAGAGTGATGTGTGGTCTTTCGGGGTG
CTCCTGTGGGAGATCTTCACTCTGGGCGGCTCCCCATACCCCGGTGTGCC
TGTGGAGGAACTTTTCAAGCTGCTGAAGGAGGGTCACCGCATGGACAAGC
CCAGTAACTGCACCAACGAGCTGTACATGATGATGCGGGACTGCTGGCAT
GCAGTGCCCTCACAGAGACCCACCTTCAAGCAGCTGGTGGAAGACCTGGA
CCGCATCGTGGCCTTGACCTCCAACCAGGAGTACCTGGACCTGTCCATGC
CCCTGGACCAGTACTCCCCCAGCTTTCCCGACACCCGGAGCTCTACGTGC
TCCTCAGGGGAGGATTCCGTCTTCTCTCATGAGCCGCTGCCCGAGGAGCC
CTGCCTGCCCCGACACCCAGCCCAGCTTGCCAATGGCGGACTCAAACGCC
GCTGACTGCCACCCACACGCCCTCCCCAGACTCCACCGTCAGCTGTAACC
CTCACCCACAGCCCCTGCTGGGCCCACCACCTGTCCGTCCCTGTCCCCTT
TCCTGCTGGCAGGAGCCGGCTGCCTACCAGGGGCCTTCCTGTGTGGCCTG
CCTTCACCCCACTCAGCTCACCTCTCCCTCCACCTCCTCTCCACCTGCTG
GTGAGAGGTGCAAAGAGGCAGATCTTTGCTGCCAGCCACTTCATCCCCTC
CCAGATGTTGGACCAACACCCCTCCCTGCCACCAGGCACTGCCTGGAGGG
CAGGGAGTGGGAGCCAATGAACAGGCATGCAAGTGAGAGCTTCCTGAGCT
TTCTCCTGTCGGTTTGGTCTGTTTTGCCTTCACCCATAAGCCCCTCGCAC
TCTGGTGGCAGGTGCCTTGTCCTCAGGGCTACAGCAGTAGGGAGGTCAGT
GCTTCGTGCCTCGATTGAAGGTGACCTCTGCCCCAGATAGGTGGTGCCAG
TGGCTTATTAATTCCGATACTAGTTTGCTTTGCTGACCAAATGCCTGGTA
CCAGAGGATGGTGAGGCGAAGGCCAGGTTGGGGGCAGTGTTGTGGCCCTG
GGGCCCAGCCCCAAACTGGGGGCTCTGTATATAGCTATGAAGAAAACACA
AAGTGTATAAATCTGAGTATATATTTACATGTCTTTTTAAAAGGGTCGTT
ACCAGAGATTTACCCATCGGGTAAGATGCTCCTGGTGGCTGGGAGGCATC
AGTTGCTATATATTAAAAACAAAAAAGAAAAAAAAGGAAAATGTTTTTAA
AAAGGTCATATATTTTTTGCTACTTTTGCTGTTTTATTTTTTTAAATTAT
GTTCTAAACCTATTTTCAGTTTAGGTCCCTCAATAAAAATTGCTGCTGCT
TCATTTATCTATGGGCTGTATGAAAAGGGTGGGAATGTCCACTGGAAAGA
AGGGACACCCACGGGCCCTGGGGCTAGGTCTGTCCCGAGGGCACCGCATG
CTCCCGGCGCAGGTTCCTTGTAACCTCTTCTTCCTAGGTCCTGCACCCAG
ACCTCACGACGCACCTCCTGCCTCTCCGCTGCTTTTGGAAAGTCAGAAAA
AGAAGATGTCTGCTTCGAGGGCAGGAACCCCATCCATGCAGTAGAGGCGC
TGGGCAGAGAGTCAAGGCCCAGCAGCCATCGACCATGGATGGTTTCCTCC
AAGGAAACCGGTGGGGTTGGGCTGGGGAGGGGGCACCTACCTAGGAATAG
CCACGGGGTAGAGCTACAGTGATTAAGAGGAAAGCAAGGGCGCGGTTGCT
CACGCCTGTAATCCCAGCACTTTGGGACACCGAGGTGGGCAGATCACTTC
AGGTCAGGAGTTTGAGACCAGCCTGGCCAACTTAGTGAAACCCCATCTCT
ACTAAAAATGCAAAAATTATCCAGGCATGGTGGCACACGCCTGTAATCCC
AGCTCCACAGGAGGCTGAGGCAGAATCCCTTGAAGCTGGGAGGCGGAGGT
TGCAGTGAGCCGAGATTGCGCCATTGCACTCCAGCCTGGGCAACAGAGAA
AACAAAAAGGAAAACAAATGATGAAGGTCTGCAGAAACTGAAACCCAGAC
ATGTGTCTGCCCCCTCTATGTGGGCATGGTTTTGCCAGTGCTTCTAAGTG
CAGGAGAACATGTCACCTGAGGCTAGTTTTGCATTCAGGTCCCTGGCTTC
GTTTCTTGTTGGTATGCCTCCCCAGATCGTCCTTCCTGTATCCATGTGAC
CAGACTGTATTTGTTGGGACTGTCGCAGATCTTGGCTTCTTACAGTTCTT
CCTGTCCAAACTCCATCCTGTCCCTCAGGAACGGGGGGAAAATTCTCCGA
ATGTTTTTGGTTTTTTGGCTGCTTGGAATTTACTTCTGCCACCTGCTGGT
CATCACTGTCCTCACTAAGTGGATTCTGGCTCCCCCGTACCTCATGGCTC
AAACTACCACTCCTCAGTCGCTATATTAAAGCTTATATTTTGCTGGATTA
CTGCTAAATACAAAAGAAAGTTCAATATGTTTTCATTTCTGTAGGGAAAA
TGGGATTGCTGCTTTAAATTTCTGAGCTAGGGATTTTTTGGCAGCTGCAG
TGTTGGCGACTATTGTAAAATTCTCTTTGTTTCTCTCTGTAAATAGCACC
TGCTAACATTACAATTTGTATTTATGTTTAAAGAAGGCATCATTTGGTGA
ACAGAACTAGGAAATGAATTTTTAGCTCTTAAAAGCATTTGCTTTGAGAC
CGCACAGGAGTGTCTTTCCTTGTAAAACAGTGATGATAATTTCTGCCTTG
GCCCTACCTTGAAGCAATGTTGTGTGAAGGGATGAAGAATCTAAAAGTCT
TCATAAGTCCTTGGGAGAGGTGCTAGAAAAATATAAGGCACTATCATAAT
TACAGTGATGTCCTTGCTGTTACTACTCAAATCACCCACAAATTTCCCCA
AAGACTGCGCTAGCTGTCAAATAAAAGACAGTGAAATTGACCTGAAAAAA
AAAAAAAAAAA

In some aspects, provided herein are rearrangements involving an FGFR2 gene, as well as FGFR2 fusion nucleic acid molecules and polypeptides.

As used herein “Fibroblast growth factor receptor 2” or “FGFR2” refer to a gene encoding an FGFR2 mRNA or polypeptide. The FGFR2 gene encodes the FGFR2 receptor tyrosine kinase protein. FGFR2 is also known as BBDS, BEK, BFR-1, CD332, CEK3, CFD1, ECT1, JWS, K-SAM, KGFR, TK14, TK25, Fibroblast growth factor receptor 2, and FGFR2 receptor tyrosine kinase. In some embodiments, an FGFR2 gene is a human FGFR2 gene. An exemplary FGFR2 gene is represented by NCBI Gene ID No. 2263. An exemplary FGFR2 mRNA sequence is represented by NCBI Ref. Seq. NM_000141, provided below as SEQ ID NO: 6. An exemplary amino acid sequence of an FGFR2 polypeptide is represented by NCBI Ref. Seq. NP_000132.

(SEQ ID NO: 6)
GAGAGCGCGGTGGAGAGCCGAGCGGGCGGGCGGCGGGTGCGGAGCGGGCG
AGGGAGCGCGCGCGGCCGCCACAAAGCTCGGGCGCCGCGGGGCTGCATGC
GGCGTACCTGGCCCGGCGCGGCGACTGCTCTCCGGGCTGGCGGGGGCCGG
CCGCGAGCCCCGGGGGCCCCGAGGCCGCAGCTTGCCTGCGCGCTCTGAGC
CTTCGCAACTCGCGAGCAAAGTTTGGTGGAGGCAACGCCAAGCCTGAGTC
CTTTCTTCCTCTCGTTCCCCAAATCCGAGGGCAGCCCGCGGGCGTCATGC
CCGCGCTCCTCCGCAGCCTGGGGTACGCGTGAAGCCCGGGAGGCTTGGCG
CCGGCGAAGACCCAAGGACCACTCTTCTGCGTTTGGAGTTGCTCCCCGCA
ACCCCGGGCTCGTCGCTTTCTCCATCCCGACCCACGCGGGGCGCGGGGAC
AACACAGGTCGCGGAGGAGCGTTGCCATTCAAGTGACTGCAGCAGCAGCG
GCAGCGCCTCGGTTCCTGAGCCCACCGCAGGCTGAAGGCATTGCGCGTAG
TCCATGCCCGTAGAGGAAGTGTGCAGATGGGATTAACGTCCACATGGAGA
TATGGAAGAGGACCGGGGATTGGTACCGTAACCATGGTCAGCTGGGGTCG
TTTCATCTGCCTGGTCGTGGTCACCATGGCAACCTTGTCCCTGGCCCGGC
CCTCCTTCAGTTTAGTTGAGGATACCACATTAGAGCCAGAAGAGCCACCA
ACCAAATACCAAATCTCTCAACCAGAAGTGTACGTGGCTGCGCCAGGGGA
GTCGCTAGAGGTGCGCTGCCTGTTGAAAGATGCCGCCGTGATCAGTTGGA
CTAAGGATGGGGTGCACTTGGGGCCCAACAATAGGACAGTGCTTATTGGG
GAGTACTTGCAGATAAAGGGCGCCACGCCTAGAGACTCCGGCCTCTATGC
TTGTACTGCCAGTAGGACTGTAGACAGTGAAACTTGGTACTTCATGGTGA
ATGTCACAGATGCCATCTCATCCGGAGATGATGAGGATGACACCGATGGT
GCGGAAGATTTTGTCAGTGAGAACAGTAACAACAAGAGAGCACCATACTG
GACCAACACAGAAAAGATGGAAAAGCGGCTCCATGCTGTGCCTGCGGCCA
ACACTGTCAAGTTTCGCTGCCCAGCCGGGGGGAACCCAATGCCAACCATG
CGGTGGCTGAAAAACGGGAAGGAGTTTAAGCAGGAGCATCGCATTGGAGG
CTACAAGGTACGAAACCAGCACTGGAGCCTCATTATGGAAAGTGTGGTCC
CATCTGACAAGGGAAATTATACCTGTGTAGTGGAGAATGAATACGGGTCC
ATCAATCACACGTACCACCTGGATGTTGTGGAGCGATCGCCTCACCGGCC
CATCCTCCAAGCCGGACTGCCGGCAAATGCCTCCACAGTGGTCGGAGGAG
ACGTAGAGTTTGTCTGCAAGGTTTACAGTGATGCCCAGCCCCACATCCAG
TGGATCAAGCACGTGGAAAAGAACGGCAGTAAATACGGGCCCGACGGGCT
GCCCTACCTCAAGGTTCTCAAGGCCGCCGGTGTTAACACCACGGACAAAG
AGATTGAGGTTCTCTATATTCGGAATGTAACTTTTGAGGACGCTGGGGAA
TATACGTGCTTGGCGGGTAATTCTATTGGGATATCCTTTCACTCTGCATG
GTTGACAGTTCTGCCAGCGCCTGGAAGAGAAAAGGAGATTACAGCTTCCC
CAGACTACCTGGAGATAGCCATTTACTGCATAGGGGTCTTCTTAATCGCC
TGTATGGTGGTAACAGTCATCCTGTGCCGAATGAAGAACACGACCAAGAA
GCCAGACTTCAGCAGCCAGCCGGCTGTGCACAAGCTGACCAAACGTATCC
CCCTGCGGAGACAGGTAACAGTTTCGGCTGAGTCCAGCTCCTCCATGAAC
TCCAACACCCCGCTGGTGAGGATAACAACACGCCTCTCTTCAACGGCAGA
CACCCCCATGCTGGCAGGGGTCTCCGAGTATGAACTTCCAGAGGACCCAA
AATGGGAGTTTCCAAGAGATAAGCTGACACTGGGCAAGCCCCTGGGAGAA
GGTTGCTTTGGGCAAGTGGTCATGGCGGAAGCAGTGGGAATTGACAAAGA
CAAGCCCAAGGAGGCGGTCACCGTGGCCGTGAAGATGTTGAAAGATGATG
CCACAGAGAAAGACCTTTCTGATCTGGTGTCAGAGATGGAGATGATGAAG
ATGATTGGGAAACACAAGAATATCATAAATCTTCTTGGAGCCTGCACACA
GGATGGGCCTCTCTATGTCATAGTTGAGTATGCCTCTAAAGGCAACCTCC
GAGAATACCTCCGAGCCCGGAGGCCACCCGGGATGGAGTACTCCTATGAC
ATTAACCGTGTTCCTGAGGAGCAGATGACCTTCAAGGACTTGGTGTCATG
CACCTACCAGCTGGCCAGAGGCATGGAGTACTTGGCTTCCCAAAAATGTA
TTCATCGAGATTTAGCAGCCAGAAATGTTTTGGTAACAGAAAACAATGTG
ATGAAAATAGCAGACTTTGGACTCGCCAGAGATATCAACAATATAGACTA
TTACAAAAAGACCACCAATGGGCGGCTTCCAGTCAAGTGGATGGCTCCAG
AAGCCCTGTTTGATAGAGTATACACTCATCAGAGTGATGTCTGGTCCTTC
GGGGTGTTAATGTGGGAGATCTTCACTTTAGGGGGCTCGCCCTACCCAGG
GATTCCCGTGGAGGAACTTTTTAAGCTGCTGAAGGAAGGACACAGAATGG
ATAAGCCAGCCAACTGCACCAACGAACTGTACATGATGATGAGGGACTGT
TGGCATGCAGTGCCCTCCCAGAGACCAACGTTCAAGCAGTTGGTAGAAGA
CTTGGATCGAATTCTCACTCTCACAACCAATGAGGAATACTTGGACCTCA
GCCAACCTCTCGAACAGTATTCACCTAGTTACCCTGACACAAGAAGTTCT
TGTTCTTCAGGAGATGATTCTGTTTTTTCTCCAGACCCCATGCCTTACGA
ACCATGCCTTCCTCAGTATCCACACATAAACGGCAGTGTTAAAACATGAA
TGACTGTGTCTGCCTGTCCCCAAACAGGACAGCACTGGGAACCTAGCTAC
ACTGAGCAGGGAGACCATGCCTCCCAGAGCTTGTTGTCTCCACTTGTATA
TATGGATCAGAGGAGTAAATAATTGGAAAAGTAATCAGCATATGTGTAAA
GATTTATACAGTTGAAAACTTGTAATCTTCCCCAGGAGGAGAAGAAGGTT
TCTGGAGCAGTGGACTGCCACAAGCCACCATGTAACCCCTCTCACCTGCC
GTGCGTACTGGCTGTGGACCAGTAGGACTCAAGGTGGACGTGCGTTCTGC
CTTCCTTGTTAATTTTGTAATAATTGGAGAAGATTTATGTCAGCACACAC
TTACAGAGCACAAATGCAGTATATAGGTGCTGGATGTATGTAAATATATT
CAAATTATGTATAAATATATATTATATATTTACAAGGAGTTATTTTTTGT
ATTGATTTTAAATGGATGTCCCAATGCACCTAGAAAATTGGTCTCTCTTT
TTTTAATAGCTATTTGCTAAATGCTGTTCTTACACATAATTTCTTAATTT
TCACCGAGCAGAGGTGGAAAAATACTTTTGCTTTCAGGGAAAATGGTATA
ACGTTAATTTATTAATAAATTGGTAATATACAAAACAATTAATCATTTAT
AGTTTTTTTTGTAATTTAAGTGGCATTTCTATGCAGGCAGCACAGCAGAC
TAGTTAATCTATTGCTTGGACTTAACTAGTTATCAGATCCTTTGAAAAGA
GAATATTTACAATATATGACTAATTTGGGGAAAATGAAGTTTTGATTTAT
TTGTGTTTAAATGCTGCTGTCAGACGATTGTTCTTAGACCTCCTAAATGC
CCCATATTAAAAGAACTCATTCATAGGAAGGTGTTTCATTTTGGTGTGCA
ACCCTGTCATTACGTCAACGCAACGTCTAACTGGACTTCCCAAGATAAAT
GGTACCAGCGTCCTCTTAAAAGATGCCTTAATCCATTCCTTGAGGACAGA
CCTTAGTTGAAATGATAGCAGAATGTGCTTCTCTCTGGCAGCTGGCCTTC
TGCTTCTGAGTTGCACATTAATCAGATTAGCCTGTATTCTCTTCAGTGAA
TTTTGATAATGGCTTCCAGACTCTTTGGCGTTGGAGACGCCTGTTAGGAT
CTTCAAGTCCCATCATAGAAAATTGAAACACAGAGTTGTTCTGCTGATAG
TTTTGGGGATACGTCCATCTTTTTAAGGGATTGCTTTCATCTAATTCTGG
CAGGACCTCACCAAAAGATCCAGCCTCATACCTACATCAGACAAAATATC
GCCGTTGTTCCTTCTGTACTAAAGTATTGTGTTTTGCTTTGGAAACACCC
ACTCACTTTGCAATAGCCGTGCAAGATGAATGCAGATTACACTGATCTTA
TGTGTTACAAAATTGGAGAAAGTATTTAATAAAACCTGTTAATTTTTATA
CTGACAATAAAAATGTTTCTACAGATATTAATGTTAACAAGACAAAATAA
ATGTCACGCAACTTATTTTTTTAA

In some aspects, provided herein are rearrangements involving an FGFR3 gene, as well as FGFR3 fusion nucleic acid molecules and polypeptides.

As used herein “Fibroblast growth factor receptor 3” or “FGFR3” refer to a gene encoding an FGFR3 mRNA or polypeptide. The FGFR3 gene encodes the FGFR3 receptor tyrosine kinase protein. FGFR3 is also known as ACH, CEK2, JTK4, CD333, HSFGFR3EX, Fibroblast growth factor receptor 3, and FGFR3 receptor tyrosine kinase. In some embodiments, an FGFR3 gene is a human FGFR3 gene. An exemplary FGFR3 gene is represented by NCBI Gene ID No. 2261. An exemplary FGFR3 mRNA sequence is represented by NCBI Ref. Seq. NM_000142, provided below as SEQ ID NO: 7. An exemplary amino acid sequence of an FGFR3 polypeptide is represented by NCBI Ref. Seq. NP_000133.

(SEQ ID NO: 7)
AGTGCGCGGTGGCGGCGGCGTCGCGGGCAGCTGGCGCCGCGCGGTCCTGC
TCTGCCGGTCGCACGGACGCACCGGCGGGCCGCCGGCCGGAGGGACGGGG
GGGGAGCTGGGCCCGCGGACAGCGAGCCGGAGCGGGAGCCGCGCGTAGCG
AGCCGGGCTCCGGCGCTCGCCAGTCTCCCGAGCGGCGCCCGCCTCCCGCC
GGTGCCCGCGCCGGGCCGTGGGGGGCAGCATGCCCGCGCGCGCTGCCTGA
GGACGCCGCGGCCCCCGCCCCCGCCATGGGCGCCCCTGCCTGCGCCCTCG
CGCTCTGCGTGGCCGTGGCCATCGTGGCCGGCGCCTCCTCGGAGTCCTTG
GGGACGGAGCAGCGCGTCGTGGGGCGAGCGGCAGAAGTCCCGGGCCCAGA
GCCCGGCCAGCAGGAGCAGTTGGTCTTCGGCAGCGGGGATGCTGTGGAGC
TGAGCTGTCCCCCGCCCGGGGGTGGTCCCATGGGGCCCACTGTCTGGGTC
AAGGATGGCACAGGGCTGGTGCCCTCGGAGCGTGTCCTGGTGGGGCCCCA
GCGGCTGCAGGTGCTGAATGCCTCCCACGAGGACTCCGGGGCCTACAGCT
GCCGGCAGCGGCTCACGCAGCGCGTACTGTGCCACTTCAGTGTGCGGGTG
ACAGACGCTCCATCCTCGGGAGATGACGAAGACGGGGAGGACGAGGCTGA
GGACACAGGTGTGGACACAGGGGCCCCTTACTGGACACGGCCCGAGCGGA
TGGACAAGAAGCTGCTGGCCGTGCCGGCCGCCAACACCGTCCGCTTCCGC
TGCCCAGCCGCTGGCAACCCCACTCCCTCCATCTCCTGGCTGAAGAACGG
CAGGGAGTTCCGCGGCGAGCACCGCATTGGAGGCATCAAGCTGCGGCATC
AGCAGTGGAGCCTGGTCATGGAAAGCGTGGTGCCCTCGGACCGCGGCAAC
TACACCTGCGTCGTGGAGAACAAGTTTGGCAGCATCCGGCAGACGTACAC
GCTGGACGTGCTGGAGCGCTCCCCGCACCGGCCCATCCTGCAGGCGGGGC
TGCCGGCCAACCAGACGGCGGTGCTGGGCAGCGACGTGGAGTTCCACTGC
AAGGTGTACAGTGACGCACAGCCCCACATCCAGTGGCTCAAGCACGTGGA
GGTGAATGGCAGCAAGGTGGGCCCGGACGGCACACCCTACGTTACCGTGC
TCAAGACGGCGGGCGCTAACACCACCGACAAGGAGCTAGAGGTTCTCTCC
TTGCACAACGTCACCTTTGAGGACGCCGGGGAGTACACCTGCCTGGCGGG
CAATTCTATTGGGTTTTCTCATCACTCTGCGTGGCTGGTGGTGCTGCCAG
CCGAGGAGGAGCTGGTGGAGGCTGACGAGGCGGGCAGTGTGTATGCAGGC
ATCCTCAGCTACGGGGTGGGCTTCTTCCTGTTCATCCTGGTGGTGGCGGC
TGTGACGCTCTGCCGCCTGCGCAGCCCCCCCAAGAAAGGCCTGGGCTCCC
CCACCGTGCACAAGATCTCCCGCTTCCCGCTCAAGCGACAGGTGTCCCTG
GAGTCCAACGCGTCCATGAGCTCCAACACACCACTGGTGCGCATCGCAAG
GCTGTCCTCAGGGGAGGGCCCCACGCTGGCCAATGTCTCCGAGCTCGAGC
TGCCTGCCGACCCCAAATGGGAGCTGTCTCGGGCCCGGCTGACCCTGGGC
AAGCCCCTTGGGGAGGGCTGCTTCGGCCAGGTGGTCATGGCGGAGGCCAT
CGGCATTGACAAGGACCGGGCCGCCAAGCCTGTCACCGTAGCCGTGAAGA
TGCTGAAAGACGATGCCACTGACAAGGACCTGTCGGACCTGGTGTCTGAG
ATGGAGATGATGAAGATGATCGGGAAACACAAAAACATCATCAACCTGCT
GGGCGCCTGCACGCAGGGCGGGCCCCTGTACGTGCTGGTGGAGTACGCGG
CCAAGGGTAACCTGCGGGAGTTTCTGCGGGCGCGGCGGCCCCCGGGCCTG
GACTACTCCTTCGACACCTGCAAGCCGCCCGAGGAGCAGCTCACCTTCAA
GGACCTGGTGTCCTGTGCCTACCAGGTGGCCCGGGGCATGGAGTACTTGG
CCTCCCAGAAGTGCATCCACAGGGACCTGGCTGCCCGCAATGTGCTGGTG
ACCGAGGACAACGTGATGAAGATCGCAGACTTCGGGCTGGCCCGGGACGT
GCACAACCTCGACTACTACAAGAAGACGACCAACGGCCGGCTGCCCGTGA
AGTGGATGGCGCCTGAGGCCTTGTTTGACCGAGTCTACACTCACCAGAGT
GACGTCTGGTCCTTTGGGGTCCTGCTCTGGGAGATCTTCACGCTGGGGGG
CTCCCCGTACCCCGGCATCCCTGTGGAGGAGCTCTTCAAGCTGCTGAAGG
AGGGCCACCGCATGGACAAGCCCGCCAACTGCACACACGACCTGTACATG
ATCATGCGGGAGTGCTGGCATGCCGCGCCCTCCCAGAGGCCCACCTTCAA
GCAGCTGGTGGAGGACCTGGACCGTGTCCTTACCGTGACGTCCACCGACG
AGTACCTGGACCTGTCGGCGCCTTTCGAGCAGTACTCCCCGGGTGGCCAG
GACACCCCCAGCTCCAGCTCCTCAGGGGACGACTCCGTGTTTGCCCACGA
CCTGCTGCCCCCGGCCCCACCCAGCAGTGGGGGCTCGCGGACGTGAAGGG
CCACTGGTCCCCAACAATGTGAGGGGTCCCTAGCAGCCCACCCTGCTGCT
GGTGCACAGCCACTCCCCGGCATGAGACTCAGTGCAGATGGAGAGACAGC
TACACAGAGCTTTGGTCTGTGTGTGTGTGTGTGCGTGTGTGTGTGTGTGT
GTGCACATCCGCGTGTGCCTGTGTGCGTGCGCATCTTGCCTCCAGGTGCA
GAGGTACCCTGGGTGTCCCCGCTGCTGTGCAACGGTCTCCTGACTGGTGC
TGCAGCACCGAGGGGCCTTTGTTCTGGGGGGACCCAGTGCAGAATGTAAG
TGGGCCCACCCGGTGGGACCCCCGTGGGGCAGGGAGCTGGGCCCGACATG
GCTCCGGCCTCTGCCTTTGCACCACGGGACATCACAGGGTGGGCCTCGGC
CCCTCCCACACCCAAAGCTGAGCCTGCAGGGAAGCCCCACATGTCCAGCA
CCTTGTGCCTGGGGTGTTAGTGGCACCGCCTCCCCACCTCCAGGCTTTCC
CACTTCCCACCCTGCCCCTCAGAGACTGAAATTACGGGTACCTGAAGATG
GGAGCCTTTACCTTTTATGCAAAAGGTTTATTCCGGAAACTAGTGTACAT
TTCTATAAATAGATGCTGTGTATATGGTATATATACATATATATATATAA
CATATATGGAAGAGGAAAAGGCTGGTACAACGGAGGCCTGCGACCCTGGG
GGCACAGGAGGCAGGCATGGCCCTGGGCGGGGCGTGGGGGGGCGTGGAGG
GAGGCCCCAGGGGGTCTCACCCATGCAAGCAGAGGACCAGGGCCTTTTCT
GGCACCGCAGTTTTGTTTTAAAACTGGACCTGTATATTTGTAAAGCTATT
TATGGGCCCCTGGCACTCTTGTTCCCACACCCCAACACTTCCAGCATTTA
GCTGGCCACATGGCGGAGAGTTTTAATTTTTAACTTATTGACAACCGAGA
AGGTTTATCCCGCCGATAGAGGGACGGCCAAGAATGTACGTCCAGCCTGC
CCCGGAGCTGGAGGATCCCCTCCAAGCCTAAAAGGTTGTTAATAGTTGGA
GGTGATTCCAGTGAAGATATTTTATTTCCTTTGTCCTTTTTCAGGAGAAT
TAGATTTCTATAGGATTTTTCTTTAGGAGATTTATTTTTTGGACTTCAAA
GCAAGCTGGTATTTTCATACAAATTCTTCTAATTGCTGTGTGTCCCAGGC
AGGGAGACGGTTTCCAGGGAGGGGCCGGCCCTGTGTGCAGGTTCCGATGT
TATTAGATGTTACAAGTTTATATATATCTATATATATAATTTATTGAGTT
TTTACAAGATGTATTTGTTGTAGACTTAACACTTCTTACGCAATGCTTCT
AGAGTTTTATAGCCTGGACTGCTACCTTTCAAAGCTTGGAGGGAAGCCGT
GAATTCAGTTGGTTCGTTCTGTACTGTTACTGGGCCCTGAGTCTGGGCAG
CTGTCCCTTGCTTGCCTGCAGGGCCATGGCTCAGGGTGGTCTCTTCTTGG
GGCCCAGTGCATGGTGGCCAGAGGTGTCACCCAAACCGGCAGGTGCGATT
TTGTTAACCCAGCGACGAACTTTCCGAAAAATAAAGACACCTGGTTGCTA
A

In some aspects, provided herein are rearrangements involving a MET gene, as well as MET fusion nucleic acid molecules and polypeptides.

As used herein “Mesenchymal Epithelial Transition” or “MET” refer to a gene encoding a MET mRNA or polypeptide. The MET gene encodes the MET receptor tyrosine kinase protein. MET is also known as HGFR, AUTS9, RCCP2, c-Met, DFNB97, Mesenchymal Epithelial Transition, and MET receptor tyrosine kinase. In some embodiments, a MET gene is a human MET gene. An exemplary MET gene is represented by NCBI Gene ID No. 4233. An exemplary MET mRNA sequence is represented by NCBI Ref. Seq. NM_000245, provided below as SEQ ID NO: 8. An exemplary amino acid sequence of a MET polypeptide is represented by NCBI Ref. Seq. NP_000236.

(SEQ ID NO: 8)
AGACACGTGCTGGGGGGGGCAGGCGAGCGCCTCAGTCTGGTCGCCTGGCG
GTGCCTCCGGCCCCAACGCGCCCGGGCCGCCGCGGGCCGCGCGCGCCGAT
GCCCGGCTGAGTCACTGGCAGGGCAGCGCGCGTGTGGGAAGGGGCGGAGG
GAGTGCGGCCGGCGGGCGGGCGGGGCGCTGGGCTCAGCCCGGCCGCAGGT
GACCCGGAGGCCCTCGCCGCCCGCGGCGCCCCGAGCGCTTTGTGAGCAGA
TGCGGAGCCGAGTGGAGGGCGCGAGCCAGATGCGGGGCGACAGCTGACTT
GCTGAGAGGAGGCGGGGAGGCGCGGAGCGCGCGTGTGGTCCTTGCGCCGC
TGACTTCTCCACTGGTTCCTGGGCACCGAAAGATAAACCTCTCATAATGA
AGGCCCCCGCTGTGCTTGCACCTGGCATCCTCGTGCTCCTGTTTACCTTG
GTGCAGAGGAGCAATGGGGAGTGTAAAGAGGCACTAGCAAAGTCCGAGAT
GAATGTGAATATGAAGTATCAGCTTCCCAACTTCACCGCGGAAACACCCA
TCCAGAATGTCATTCTACATGAGCATCACATTTTCCTTGGTGCCACTAAC
TACATTTATGTTTTAAATGAGGAAGACCTTCAGAAGGTTGCTGAGTACAA
GACTGGGCCTGTGCTGGAACACCCAGATTGTTTCCCATGTCAGGACTGCA
GCAGCAAAGCCAATTTATCAGGAGGTGTTTGGAAAGATAACATCAACATG
GCTCTAGTTGTCGACACCTACTATGATGATCAACTCATTAGCTGTGGCAG
CGTCAACAGAGGGACCTGCCAGCGACATGTCTTTCCCCACAATCATACTG
CTGACATACAGTCGGAGGTTCACTGCATATTCTCCCCACAGATAGAAGAG
CCCAGCCAGTGTCCTGACTGTGTGGTGAGCGCCCTGGGAGCCAAAGTCCT
TTCATCTGTAAAGGACCGGTTCATCAACTTCTTTGTAGGCAATACCATAA
ATTCTTCTTATTTCCCAGATCATCCATTGCATTCGATATCAGTGAGAAGG
CTAAAGGAAACGAAAGATGGTTTTATGTTTTTGACGGACCAGTCCTACAT
TGATGTTTTACCTGAGTTCAGAGATTCTTACCCCATTAAGTATGTCCATG
CCTTTGAAAGCAACAATTTTATTTACTTCTTGACGGTCCAAAGGGAAACT
CTAGATGCTCAGACTTTTCACACAAGAATAATCAGGTTCTGTTCCATAAA
CTCTGGATTGCATTCCTACATGGAAATGCCTCTGGAGTGTATTCTCACAG
AAAAGAGAAAAAAGAGATCCACAAAGAAGGAAGTGTTTAATATACTTCAG
GCTGCGTATGTCAGCAAGCCTGGGGCCCAGCTTGCTAGACAAATAGGAGC
CAGCCTGAATGATGACATTCTTTTCGGGGTGTTCGCACAAAGCAAGCCAG
ATTCTGCCGAACCAATGGATCGATCTGCCATGTGTGCATTCCCTATCAAA
TATGTCAACGACTTCTTCAACAAGATCGTCAACAAAAACAATGTGAGATG
TCTCCAGCATTTTTACGGACCCAATCATGAGCACTGCTTTAATAGGACAC
TTCTGAGAAATTCATCAGGCTGTGAAGCGCGCCGTGATGAATATCGAACA
GAGTTTACCACAGCTTTGCAGCGCGTTGACTTATTCATGGGTCAATTCAG
CGAAGTCCTCTTAACATCTATATCCACCTTCATTAAAGGAGACCTCACCA
TAGCTAATCTTGGGACATCAGAGGGTCGCTTCATGCAGGTTGTGGTTTCT
CGATCAGGACCATCAACCCCTCATGTGAATTTTCTCCTGGACTCCCATCC
AGTGTCTCCAGAAGTGATTGTGGAGCATACATTAAACCAAAATGGCTACA
CACTGGTTATCACTGGGAAGAAGATCACGAAGATCCCATTGAATGGCTTG
GGCTGCAGACATTTCCAGTCCTGCAGTCAATGCCTCTCTGCCCCACCCTT
TGTTCAGTGTGGCTGGTGCCACGACAAATGTGTGCGATCGGAGGAATGCC
TGAGCGGGACATGGACTCAACAGATCTGTCTGCCTGCAATCTACAAGGTT
TTCCCAAATAGTGCACCCCTTGAAGGAGGGACAAGGCTGACCATATGTGG
CTGGGACTTTGGATTTCGGAGGAATAATAAATTTGATTTAAAGAAAACTA
GAGTTCTCCTTGGAAATGAGAGCTGCACCTTGACTTTAAGTGAGAGCACG
ATGAATACATTGAAATGCACAGTTGGTCCTGCCATGAATAAGCATTTCAA
TATGTCCATAATTATTTCAAATGGCCACGGGACAACACAATACAGTACAT
TCTCCTATGTGGATCCTGTAATAACAAGTATTTCGCCGAAATACGGTCCT
ATGGCTGGTGGCACTTTACTTACTTTAACTGGAAATTACCTAAACAGTGG
GAATTCTAGACACATTTCAATTGGTGGAAAAACATGTACTTTAAAAAGTG
TGTCAAACAGTATTCTTGAATGTTATACCCCAGCCCAAACCATTTCAACT
GAGTTTGCTGTTAAATTGAAAATTGACTTAGCCAACCGAGAGACAAGCAT
CTTCAGTTACCGTGAAGATCCCATTGTCTATGAAATTCATCCAACCAAAT
CTTTTATTAGTGGTGGGAGCACAATAACAGGTGTTGGGAAAAACCTGAAT
TCAGTTAGTGTCCCGAGAATGGTCATAAATGTGCATGAAGCAGGAAGGAA
CTTTACAGTGGCATGTCAACATCGCTCTAATTCAGAGATAATCTGTTGTA
CCACTCCTTCCCTGCAACAGCTGAATCTGCAACTCCCCCTGAAAACCAAA
GCCTTTTTCATGTTAGATGGGATCCTTTCCAAATACTTTGATCTCATTTA
TGTACATAATCCTGTGTTTAAGCCTTTTGAAAAGCCAGTGATGATCTCAA
TGGGCAATGAAAATGTACTGGAAATTAAGGGAAATGATATTGACCCTGAA
GCAGTTAAAGGTGAAGTGTTAAAAGTTGGAAATAAGAGCTGTGAGAATAT
ACACTTACATTCTGAAGCCGTTTTATGCACGGTCCCCAATGACCTGCTGA
AATTGAACAGCGAGCTAAATATAGAGTGGAAGCAAGCAATTTCTTCAACC
GTCCTTGGAAAAGTAATAGTTCAACCAGATCAGAATTTCACAGGATTGAT
TGCTGGTGTTGTCTCAATATCAACAGCACTGTTATTACTACTTGGGTTTT
TCCTGTGGCTGAAAAAGAGAAAGCAAATTAAAGATCTGGGCAGTGAATTA
GTTCGCTACGATGCAAGAGTACACACTCCTCATTTGGATAGGCTTGTAAG
TGCCCGAAGTGTAAGCCCAACTACAGAAATGGTTTCAAATGAATCTGTAG
ACTACCGAGCTACTTTTCCAGAAGATCAGTTTCCTAATTCATCTCAGAAC
GGTTCATGCCGACAAGTGCAGTATCCTCTGACAGACATGTCCCCCATCCT
AACTAGTGGGGACTCTGATATATCCAGTCCATTACTGCAAAATACTGTCC
ACATTGACCTCAGTGCTCTAAATCCAGAGCTGGTCCAGGCAGTGCAGCAT
GTAGTGATTGGGCCCAGTAGCCTGATTGTGCATTTCAATGAAGTCATAGG
AAGAGGGCATTTTGGTTGTGTATATCATGGGACTTTGTTGGACAATGATG
GCAAGAAAATTCACTGTGCTGTGAAATCCTTGAACAGAATCACTGACATA
GGAGAAGTTTCCCAATTTCTGACCGAGGGAATCATCATGAAAGATTTTAG
TCATCCCAATGTCCTCTCGCTCCTGGGAATCTGCCTGCGAAGTGAAGGGT
CTCCGCTGGTGGTCCTACCATACATGAAACATGGAGATCTTCGAAATTTC
ATTCGAAATGAGACTCATAATCCAACTGTAAAAGATCTTATTGGCTTTGG
TCTTCAAGTAGCCAAAGGCATGAAATATCTTGCAAGCAAAAAGTTTGTCC
ACAGAGACTTGGCTGCAAGAAACTGTATGCTGGATGAAAAATTCACAGTC
AAGGTTGCTGATTTTGGTCTTGCCAGAGACATGTATGATAAAGAATACTA
TAGTGTACACAACAAAACAGGTGCAAAGCTGCCAGTGAAGTGGATGGCTT
TGGAAAGTCTGCAAACTCAAAAGTTTACCACCAAGTCAGATGTGTGGTCC
TTTGGCGTGCTCCTCTGGGAGCTGATGACAAGAGGAGCCCCACCTTATCC
TGACGTAAACACCTTTGATATAACTGTTTACTTGTTGCAAGGGAGAAGAC
TCCTACAACCCGAATACTGCCCAGACCCCTTATATGAAGTAATGCTAAAA
TGCTGGCACCCTAAAGCCGAAATGCGCCCATCCTTTTCTGAACTGGTGTC
CCGGATATCAGCGATCTTCTCTACTTTCATTGGGGAGCACTATGTCCATG
TGAACGCTACTTATGTGAACGTAAAATGTGTCGCTCCGTATCCTTCTCTG
TTGTCATCAGAAGATAACGCTGATGATGAGGTGGACACACGACCAGCCTC
CTTCTGGGAGACATCATAGTGCTAGTACTATGTCAAAGCAACAGTCCACA
CTTTGTCCAATGGTTTTTTCACTGCCTGACCTTTAAAAGGCCATCGATAT
TCTTTGCTCTTGCCAAAATTGCACTATTATAGGACTTGTATTGTTATTTA
AATTACTGGATTCTAAGGAATTTCTTATCTGACAGAGCATCAGAACCAGA
GGCTTGGTCCCACAGGCCACGGACCAATGGCCTGCAGCCGTGACAACACT
CCTGTCATATTGGAGTCCAAAACTTGAATTCTGGGTTGAATTTTTTAAAA
ATCAGGTACCACTTGATTTCATATGGGAAATTGAAGCAGGAAATATTGAG
GGCTTCTTGATCACAGAAAACTCAGAAGAGATAGTAATGCTCAGGACAGG
AGCGGCAGCCCCAGAACAGGCCACTCATTTAGAATTCTAGTGTTTCAAAA
CACTTTTGTGTGTTGTATGGTCAATAACATTTTTCATTACTGATGGTGTC
ATTCACCCATTAGGTAAACATTCCCTTTTAAATGTTTGTTTGTTTTTTGA
GACAGGATCTCACTCTGTTGCCAGGGCTGTAGTGCAGTGGTGTGATCATA
GCTCACTGCAACCTCCACCTCCCAGGCTCAAGCCTCCCGAATAGCTGGGA
CTACAGGCGCACACCACCATCCCCGGCTAATTTTTGTATTTTTTGTAGAG
ACGGGGTTTTGCCATGTTGCCAAGGCTGGTTTCAAACTCCTGGACTCAAG
AAATCCACCCACCTCAGCCTCCCAAAGTGCTAGGATTACAGGCATGAGCC
ACTGCGCCCAGCCCTTATAAATTTTTGTATAGACATTCCTTTGGTTGGAA
GAATATTTATAGGCAATACAGTCAAAGTTTCAAAATAGCATCACACAAAA
CATGTTTATAAATGAACAGGATGTAATGTACATAGATGACATTAAGAAAA
TTTGTATGAAATAATTTAGTCATCATGAAATATTTAGTTGTCATATAAAA
ACCCACTGTTTGAGAATGATGCTACTCTGATCTAATGAATGTGAACATGT
AGATGTTTTGTGTGTATTTTTTTAAATGAAAACTCAAAATAAGACAAGTA
ATTTGTTGATAAATATTTTTAAAGATAACTCAGCATGTTTGTAAAGCAGG
ATACATTTTACTAAAAGGTTCATTGGTTCCAATCACAGCTCATAGGTAGA
GCAAAGAAAGGGTGGATGGATTGAAAAGATTAGCCTCTGTCTCGGTGGCA
GGTTCCCACCTCGCAAGCAATTGGAAACAAAACTTTTGGGGAGTTTTATT
TTGCATTAGGGTGTGTTTTATGTTAAGCAAAACATACTTTAGAAACAAAT
GAAAAAGGCAATTGAAAATCCCAGCTATTTCACCTAGATGGAATAGCCAC
CCTGAGCAGAACTTTGTGATGCTTCATTCTGTGGAATTTTGTGCTTGCTA
CTGTATAGTGCATGTGGTGTAGGTTACTCTAACTGGTTTTGTCGACGTAA
ACATTTAAAGTGTTATATTTTTTATAAAAATGTTTATTTTTAATGATATG
AGAAAAATTTTGTTAGGCCACAAAAACACTGCACTGTGAACATTTTAGAA
AAGGTATGTCAGACTGGGATTAATGACAGCATGATTTTCAATGACTGTAA
ATTGCGATAAGGAAATGTACTGATTGCCAATACACCCCACCCTCATTACA
TCATCAGGACTTGAAGCCAAGGGTTAACCCAGCAAGCTACAAAGAGGGTG
TGTCACACTGAAACTCAATAGTTGAGTTTGGCTGTTGTTGCAGGAAAATG
ATTATAACTAAAAGCTCTCTGATAGTGCAGAGACTTACCAGAAGACACAA
GGAATTGTACTGAAGAGCTATTACAATCCAAATATTGCCGTTTCATAAAT
GTAATAAGTAATACTAATTCACAGAGTATTGTAAATGGTGGATGACAAAA
GAAAATCTGCTCTGTGGAAAGAAAGAACTGTCTCTACCAGGGTCAAGAGC
ATGAACGCATCAATAGAAAGAACTCGGGGAAACATCCCATCAACAGGACT
ACACACTTGTATATACATTCTTGAGAACACTGCAATGTGAAAATCACGTT
TGCTATTTATAAACTTGTCCTTAGATTAATGTGTCTGGACAGATTGTGGG
AGTAAGTGATTCTTCTAAGAATTAGATACTTGTCACTGCCTATACCTGCA
GCTGAACTGAATGGTACTTCGTATGTTAATAGTTGTTCTGATAAATCATG
CAATTAAAGTAAAGTGATGCAA

In some aspects, provided herein are rearrangements involving an NTRK1 gene, as well as NTRK1 fusion nucleic acid molecules and polypeptides.

As used herein “Neurotrophic Receptor Tyrosine Kinase 1” or “NTRK1” refer to a gene encoding an NTRK1 mRNA or polypeptide. The NTRK1 gene encodes the NTRK1 tyrosine kinase protein. NTRK1 is also known as MTC, TRK, TRK1, TRKA, Trk-A, p140-TrkA, and Neurotrophic Receptor Tyrosine Kinase 1. In some embodiments, an NTRK1 gene is a human NTRK1 gene. An exemplary NTRK1 gene is represented by NCBI Gene ID No. 4914. An exemplary NTRK1 mRNA sequence is represented by NCBI Ref. Seq. NM_002529, provided below as SEQ ID NO: 12. An exemplary amino acid sequence of an NTRK1 polypeptide is represented by NCBI Ref. Seq. NP_002520.

(SEQ ID NO: 12)
GGAGGCCTGGCAGCTGCAGCTGGGAGCGCACAGACGGCTGCCCCGCCTGA
GCGAGGCGGGCGCCGCCGCGATGCTGCGAGGCGGACGGCGCGGGCAGCTT
GGCTGGCACAGCTGGGCTGCGGGGCCGGGCAGCCTGCTGGCTTGGCTGAT
ACTGGCATCTGCGGGCGCCGCACCCTGCCCCGATGCCTGCTGCCCCCACG
GCTCCTCGGGACTGCGATGCACCCGGGATGGGGCCCTGGATAGCCTCCAC
CACCTGCCCGGCGCAGAGAACCTGACTGAGCTCTACATCGAGAACCAGCA
GCATCTGCAGCATCTGGAGCTCCGTGATCTGAGGGGCCTGGGGGAGCTGA
GAAACCTCACCATCGTGAAGAGTGGTCTCCGTTTCGTGGCGCCAGATGCC
TTCCATTTCACTCCTCGGCTCAGTCGCCTGAATCTCTCCTTCAACGCTCT
GGAGTCTCTCTCCTGGAAAACTGTGCAGGGCCTCTCCTTACAGGAACTGG
TCCTGTCGGGGAACCCTCTGCACTGTTCTTGTGCCCTGCGCTGGCTACAG
CGCTGGGAGGAGGAGGGACTGGGCGGAGTGCCTGAACAGAAGCTGCAGTG
TCATGGGCAAGGGCCCCTGGCCCACATGCCCAATGCCAGCTGTGGTGTGC
CCACGCTGAAGGTCCAGGTGCCCAATGCCTCGGTGGATGTGGGGGACGAC
GTGCTGCTGCGGTGCCAGGTGGAGGGGCGGGGCCTGGAGCAGGCCGGCTG
GATCCTCACAGAGCTGGAGCAGTCAGCCACGGTGATGAAATCTGGGGGTC
TGCCATCCCTGGGGCTGACCCTGGCCAATGTCACCAGTGACCTCAACAGG
AAGAACGTGACGTGCTGGGCAGAGAACGATGTGGGCCGGGCAGAGGTCTC
TGTTCAGGTCAACGTCTCCTTCCCGGCCAGTGTGCAGCTGCACACGGCGG
TGGAGATGCACCACTGGTGCATCCCCTTCTCTGTGGATGGGCAGCCGGCA
CCGTCTCTGCGCTGGCTCTTCAATGGCTCCGTGCTCAATGAGACCAGCTT
CATCTTCACTGAGTTCCTGGAGCCGGCAGCCAATGAGACCGTGCGGCACG
GGTGTCTGCGCCTCAACCAGCCCACCCACGTCAACAACGGCAACTACACG
CTGCTGGCTGCCAACCCCTTCGGCCAGGCCTCCGCCTCCATCATGGCTGC
CTTCATGGACAACCCTTTCGAGTTCAACCCCGAGGACCCCATCCCTGTCT
CCTTCTCGCCGGTGGACACTAACAGCACATCTGGAGACCCGGTGGAGAAG
AAGGACGAAACACCTTTTGGGGTCTCGGTGGCTGTGGGCCTGGCCGTCTT
TGCCTGCCTCTTCCTTTCTACGCTGCTCCTTGTGCTCAACAAATGTGGAC
GGAGAAACAAGTTTGGGATCAACCGCCCGGCTGTGCTGGCTCCAGAGGAT
GGGCTGGCCATGTCCCTGCATTTCATGACATTGGGTGGCAGCTCCCTGTC
CCCCACCGAGGGCAAAGGCTCTGGGCTCCAAGGCCACATCATCGAGAACC
CACAATACTTCAGTGATGCCTGTGTTCACCACATCAAGCGCCGGGACATC
GTGCTCAAGTGGGAGCTGGGGGAGGGCGCCTTTGGGAAGGTCTTCCTTGC
TGAGTGCCACAACCTCCTGCCTGAGCAGGACAAGATGCTGGTGGCTGTCA
AGGCACTGAAGGAGGCGTCCGAGAGTGCTCGGCAGGACTTCCAGCGTGAG
GCTGAGCTGCTCACCATGCTGCAGCACCAGCACATCGTGCGCTTCTTCGG
CGTCTGCACCGAGGGCCGCCCCCTGCTCATGGTCTTTGAGTATATGCGGC
ACGGGGACCTCAACCGCTTCCTCCGATCCCATGGACCTGATGCCAAGCTG
CTGGCTGGTGGGGAGGATGTGGCTCCAGGCCCCCTGGGTCTGGGGCAGCT
GCTGGCCGTGGCTAGCCAGGTCGCTGCGGGGATGGTGTACCTGGCGGGTC
TGCATTTTGTGCACCGGGACCTGGCCACACGCAACTGTCTAGTGGGCCAG
GGACTGGTGGTCAAGATTGGTGATTTTGGCATGAGCAGGGATATCTACAG
CACCGACTATTACCGTGTGGGAGGCCGCACCATGCTGCCCATTCGCTGGA
TGCCGCCCGAGAGCATCCTGTACCGTAAGTTCACCACCGAGAGCGACGTG
TGGAGCTTCGGCGTGGTGCTCTGGGAGATCTTCACCTACGGCAAGCAGCC
CTGGTACCAGCTCTCCAACACGGAGGCAATCGACTGCATCACGCAGGGAC
GTGAGTTGGAGCGGCCACGTGCCTGCCCACCAGAGGTCTACGCCATCATG
CGGGGCTGCTGGCAGCGGGAGCCCCAGCAACGCCACAGCATCAAGGATGT
GCACGCCCGGCTGCAAGCCCTGGCCCAGGCACCTCCTGTCTACCTGGATG
TCCTGGGCTAGGGGGCCGGCCCAGGGGCTGGGAGTGGTTAGCCGGAATAC
TGGGGCCTGCCCTCAGCATCCCCCATAGCTCCCAGCAGCCCCAGGGTGAT
CTCAAAGTATCTAATTCACCCTCAGCATGTGGGAAGGGACAGGTGGGGGC
TGGGAGTAGAGGATGTTCCTGCTTCTCTAGGCAAGGTCCCGTCATAGCAA
TTATATTTATTATCCCTTG

In some aspects, provided herein are rearrangements involving a RAF1 gene, as well as RAF1 fusion nucleic acid molecules and polypeptides.

As used herein “Rapidly Accelerated Fibrosarcoma” or “RAF1” refer to a gene encoding a RAF1 mRNA or polypeptide. The RAF1 gene encodes the RAF1 serine/threonine kinase protein. RAF1 is also known as NS5, CRAF, Raf-1, c-Raf, CMD1NN, and Rapidly Accelerated Fibrosarcoma. In some embodiments, a RAF1 gene is a human RAF1 gene. An exemplary RAF1 gene is represented by NCBI Gene ID No. 5894. An exemplary RAF1 mRNA sequence is represented by NCBI Ref. Seq. NM_002880, provided below as SEQ ID NO: 9. An exemplary amino acid sequence of a RAF1 polypeptide is represented by NCBI Ref. Seq. NP_002871.

(SEQ ID NO: 9)
AATCGCGGGCGCTTGGGCCGCCATCTTAGATGGCGGGAGTAAGAGGAAAA
CGATTGTGAGGCGGGAACGGCTTTCTGCTGCCTTTTTTGGGCCCCGAAAA
GGGTCAGCTGGCCGGGCTTTGGGGCGCGTGCCCTGAGGCGCGGAGCGCGT
TTGCTACGATGCGGGGGCTGCTCGGGGCTCCGTCCCCTGGGCTGGGGACG
CGCCGAATGTGACCGCCTCCCGCTCCCTCACCCGCCGCGGGGAGGAGGAG
CGGGCGAGAAGCTGCCGCCGAACGACAGGACGTTGGGGCGGCCTGGCTCC
CTCAGGTTTAAGAATTGTTTAAGCTGCATCAATGGAGCACATACAGGGAG
CTTGGAAGACGATCAGCAATGGTTTTGGATTCAAAGATGCCGTGTTTGAT
GGCTCCAGCTGCATCTCTCCTACAATAGTTCAGCAGTTTGGCTATCAGCG
CCGGGCATCAGATGATGGCAAACTCACAGATCCTTCTAAGACAAGCAACA
CTATCCGTGTTTTCTTGCCGAACAAGCAAAGAACAGTGGTCAATGTGCGA
AATGGAATGAGCTTGCATGACTGCCTTATGAAAGCACTCAAGGTGAGGGG
CCTGCAACCAGAGTGCTGTGCAGTGTTCAGACTTCTCCACGAACACAAAG
GTAAAAAAGCACGCTTAGATTGGAATACTGATGCTGCGTCTTTGATTGGA
GAAGAACTTCAAGTAGATTTCCTGGATCATGTTCCCCTCACAACACACAA
CTTTGCTCGGAAGACGTTCCTGAAGCTTGCCTTCTGTGACATCTGTCAGA
AATTCCTGCTCAATGGATTTCGATGTCAGACTTGTGGCTACAAATTTCAT
GAGCACTGTAGCACCAAAGTACCTACTATGTGTGTGGACTGGAGTAACAT
CAGACAACTCTTATTGTTTCCAAATTCCACTATTGGTGATAGTGGAGTCC
CAGCACTACCTTCTTTGACTATGCGTCGTATGCGAGAGTCTGTTTCCAGG
ATGCCTGTTAGTTCTCAGCACAGATATTCTACACCTCACGCCTTCACCTT
TAACACCTCCAGTCCCTCATCTGAAGGTTCCCTCTCCCAGAGGCAGAGGT
CGACATCCACACCTAATGTCCACATGGTCAGCACCACCCTGCCTGTGGAC
AGCAGGATGATTGAGGATGCAATTCGAAGTCACAGCGAATCAGCCTCACC
TTCAGCCCTGTCCAGTAGCCCCAACAATCTGAGCCCAACAGGCTGGTCAC
AGCCGAAAACCCCCGTGCCAGCACAAAGAGAGCGGGCACCAGTATCTGGG
ACCCAGGAGAAAAACAAAATTAGGCCTCGTGGACAGAGAGATTCAAGCTA
TTATTGGGAAATAGAAGCCAGTGAAGTGATGCTGTCCACTCGGATTGGGT
CAGGCTCTTTTGGAACTGTTTATAAGGGTAAATGGCACGGAGATGTTGCA
GTAAAGATCCTAAAGGTTGTCGACCCAACCCCAGAGCAATTCCAGGCCTT
CAGGAATGAGGTGGCTGTTCTGCGCAAAACACGGCATGTGAACATTCTGC
TTTTCATGGGGTACATGACAAAGGACAACCTGGCAATTGTGACCCAGTGG
TGCGAGGGCAGCAGCCTCTACAAACACCTGCATGTCCAGGAGACCAAGTT
TCAGATGTTCCAGCTAATTGACATTGCCCGGCAGACGGCTCAGGGAATGG
ACTATTTGCATGCAAAGAACATCATCCATAGAGACATGAAATCCAACAAT
ATATTTCTCCATGAAGGCTTAACAGTGAAAATTGGAGATTTTGGTTTGGC
AACAGTAAAGTCACGCTGGAGTGGTTCTCAGCAGGTTGAACAACCTACTG
GCTCTGTCCTCTGGATGGCCCCAGAGGTGATCCGAATGCAGGATAACAAC
CCATTCAGTTTCCAGTCGGATGTCTACTCCTATGGCATCGTATTGTATGA
ACTGATGACGGGGGAGCTTCCTTATTCTCACATCAACAACCGAGATCAGA
TCATCTTCATGGTGGGCCGAGGATATGCCTCCCCAGATCTTAGTAAGCTA
TATAAGAACTGCCCCAAAGCAATGAAGAGGCTGGTAGCTGACTGTGTGAA
GAAAGTAAAGGAAGAGAGGCCTCTTTTTCCCCAGATCCTGTCTTCCATTG
AGCTGCTCCAACACTCTCTACCGAAGATCAACCGGAGCGCTTCCGAGCCA
TCCTTGCATCGGGCAGCCCACACTGAGGATATCAATGCTTGCACGCTGAC
CACGTCCCCGAGGCTGCCTGTCTTCTAGTTGACTTTGCACCTGTCTTCAG
GCTGCCAGGGGAGGAGGAGAAGCCAGCAGGCACCACTTTTCTGCTCCCTT
TCTCCAGAGGCAGAACACATGTTTTCAGAGAAGCTGCTGCTAAGGACCTT
CTAGACTGCTCACAGGGCCTTAACTTCATGTTGCCTTCTTTTCTATCCCT
TTGGGCCCTGGGAGAAGGAAGCCATTTGCAGTGCTGGTGTGTCCTGCTCC
CTCCCCACATTCCCCATGCTCAAGGCCCAGCCTTCTGTAGATGCGCAAGT
GGATGTTGATGGTAGTACAAAAAGCAGGGGCCCAGCCCCAGCTGTTGGCT
ACATGAGTATTTAGAGGAAGTAAGGTAGCAGGCAGTCCAGCCCTGATGTG
GAGACACATGGGATTTTGGAAATCAGCTTCTGGAGGAATGCATGTCACAG
GCGGGACTTTCTTCAGAGAGTGGTGCAGCGCCAGACATTTTGCACATAAG
GCACCAAACAGCCCAGGACTGCCGAGACTCTGGCCGCCCGAAGGAGCCTG
CTTTGGTACTATGGAACTTTTCTTAGGGGACACGTCCTCCTTTCACAGCT
TCTAAGGTGTCCAGTGCATTGGGATGGTTTTCCAGGCAAGGCACTCGGCC
AATCCGCATCTCAGCCCTCTCAGGGAGCAGTCTTCCATCATGCTGAATTT
TGTCTTCCAGGAGCTGCCCCTATGGGGCGGGGCCGCAGGGCCAGCCTTGT
TTCTCTAACAAACAAACAAACAAACAGCCTTGTTTCTCTAGTCACATCAT
GTGTATACAAGGAAGCCAGGAATACAGGTTTTCTTGATGATTTGGGTTTT
AATTTTGTTTTTATTGCACCTGACAAAATACAGTTATCTGATGGTCCCTC
AATTATGTTATTTTAATAAAATAAATTAAATTTAGGTGTAA

In some aspects, provided herein are rearrangements involving a RET gene, as well as RET fusion nucleic acid molecules and polypeptides.

As used herein “Rearranged During Transfection” or “RET” refer to a gene encoding a RET mRNA or polypeptide. The RET gene encodes the RET receptor tyrosine kinase protein. RET is also known as PTC, MTC1, HSCR1, MEN2A, MEN2B, CDHF12, CDHR16, RET-ELE1, Rearranged During Transfection, and RET receptor tyrosine kinase. In some embodiments, a RET gene is a human RET gene. An exemplary RET gene is represented by NCBI Gene ID No. 5979. An exemplary RET mRNA sequence is represented by NCBI Ref. Seq. NM_020630, provided below as SEQ ID NO: 10. An exemplary amino acid sequence of a RET polypeptide is represented by NCBI Ref. Seq. NP_065681.

(SEQ ID NO: 10)
AGTCCCGCGACCGAAGCAGGGCGCGCAGCAGCGCTGAGTGCCCCGGAACG
TGCGTCGCGCCCCCAGTGTCCGTCGCGTCCGCCGCGCCCCGGGCGGGGAT
GGGGCGGCCAGACTGAGCGCCGCACCCGCCATCCAGACCCGCCGGCCCTA
GCCGCAGTCCCTCCAGCCGTGGCCCCAGCGCGCACGGGCGATGGCGAAGG
CGACGTCCGGTGCCGCGGGGCTGCGTCTGCTGTTGCTGCTGCTGCTGCCG
CTGCTAGGCAAAGTGGCATTGGGCCTCTACTTCTCGAGGGATGCTTACTG
GGAGAAGCTGTATGTGGACCAGGCGGCCGGCACGCCCTTGCTGTACGTCC
ATGCCCTGCGGGACGCCCCTGAGGAGGTGCCCAGCTTCCGCCTGGGCCAG
CATCTCTACGGCACGTACCGCACACGGCTGCATGAGAACAACTGGATCTG
CATCCAGGAGGACACCGGCCTCCTCTACCTTAACCGGAGCCTGGACCATA
GCTCCTGGGAGAAGCTCAGTGTCCGCAACCGCGGCTTTCCCCTGCTCACC
GTCTACCTCAAGGTCTTCCTGTCACCCACATCCCTTCGTGAGGGCGAGTG
CCAGTGGCCAGGCTGTGCCCGCGTATACTTCTCCTTCTTCAACACCTCCT
TTCCAGCCTGCAGCTCCCTCAAGCCCCGGGAGCTCTGCTTCCCAGAGACA
AGGCCCTCCTTCCGCATTCGGGAGAACCGACCCCCAGGCACCTTCCACCA
GTTCCGCCTGCTGCCTGTGCAGTTCTTGTGCCCCAACATCAGCGTGGCCT
ACAGGCTCCTGGAGGGTGAGGGTCTGCCCTTCCGCTGCGCCCCGGACAGC
CTGGAGGTGAGCACGCGCTGGGCCCTGGACCGCGAGCAGCGGGAGAAGTA
CGAGCTGGTGGCCGTGTGCACCGTGCACGCCGGCGCGCGCGAGGAGGTGG
TGATGGTGCCCTTCCCGGTGACCGTGTACGACGAGGACGACTCGGCGCCC
ACCTTCCCCGCGGGCGTCGACACCGCCAGCGCCGTGGTGGAGTTCAAGCG
GAAGGAGGACACCGTGGTGGCCACGCTGCGTGTCTTCGATGCAGACGTGG
TACCTGCATCAGGGGAGCTGGTGAGGCGGTACACAAGCACGCTGCTCCCC
GGGGACACCTGGGCCCAGCAGACCTTCCGGGTGGAACACTGGCCCAACGA
GACCTCGGTCCAGGCCAACGGCAGCTTCGTGCGGGCGACCGTACATGACT
ATAGGCTGGTTCTCAACCGGAACCTCTCCATCTCGGAGAACCGCACCATG
CAGCTGGCGGTGCTGGTCAATGACTCAGACTTCCAGGGCCCAGGAGCGGG
CGTCCTCTTGCTCCACTTCAACGTGTCGGTGCTGCCGGTCAGCCTGCACC
TGCCCAGTACCTACTCCCTCTCCGTGAGCAGGAGGGCTCGCCGATTTGCC
CAGATCGGGAAAGTCTGTGTGGAAAACTGCCAGGCATTCAGTGGCATCAA
CGTCCAGTACAAGCTGCATTCCTCTGGTGCCAACTGCAGCACGCTAGGGG
TGGTCACCTCAGCCGAGGACACCTCGGGGATCCTGTTTGTGAATGACACC
AAGGCCCTGCGGCGGCCCAAGTGTGCCGAACTTCACTACATGGTGGTGGC
CACCGACCAGCAGACCTCTAGGCAGGCCCAGGCCCAGCTGCTTGTAACAG
TGGAGGGGTCATATGTGGCCGAGGAGGCGGGCTGCCCCCTGTCCTGTGCA
GTCAGCAAGAGACGGCTGGAGTGTGAGGAGTGTGGCGGCCTGGGCTCCCC
AACAGGCAGGTGTGAGTGGAGGCAAGGAGATGGCAAAGGGATCACCAGGA
ACTTCTCCACCTGCTCTCCCAGCACCAAGACCTGCCCCGACGGCCACTGC
GATGTTGTGGAGACCCAAGACATCAACATTTGCCCTCAGGACTGCCTCCG
GGGCAGCATTGTTGGGGGACACGAGCCTGGGGAGCCCCGGGGGATTAAAG
CTGGCTATGGCACCTGCAACTGCTTCCCTGAGGAGGAGAAGTGCTTCTGC
GAGCCCGAAGACATCCAGGATCCACTGTGCGACGAGCTGTGCCGCACGGT
GATCGCAGCCGCTGTCCTCTTCTCCTTCATCGTCTCGGTGCTGCTGTCTG
CCTTCTGCATCCACTGCTACCACAAGTTTGCCCACAAGCCACCCATCTCC
TCAGCTGAGATGACCTTCCGGAGGCCCGCCCAGGCCTTCCCGGTCAGCTA
CTCCTCTTCCGGTGCCCGCCGGCCCTCGCTGGACTCCATGGAGAACCAGG
TCTCCGTGGATGCCTTCAAGATCCTGGAGGATCCAAAGTGGGAATTCCCT
CGGAAGAACTTGGTTCTTGGAAAAACTCTAGGAGAAGGCGAATTTGGAAA
AGTGGTCAAGGCAACGGCCTTCCATCTGAAAGGCAGAGCAGGGTACACCA
CGGTGGCCGTGAAGATGCTGAAAGAGAACGCCTCCCCGAGTGAGCTTCGA
GACCTGCTGTCAGAGTTCAACGTCCTGAAGCAGGTCAACCACCCACATGT
CATCAAATTGTATGGGGCCTGCAGCCAGGATGGCCCGCTCCTCCTCATCG
TGGAGTACGCCAAATACGGCTCCCTGCGGGGCTTCCTCCGCGAGAGCCGC
AAAGTGGGGCCTGGCTACCTGGGCAGTGGAGGCAGCCGCAACTCCAGCTC
CCTGGACCACCCGGATGAGCGGGCCCTCACCATGGGCGACCTCATCTCAT
TTGCCTGGCAGATCTCACAGGGGATGCAGTATCTGGCCGAGATGAAGCTC
GTTCATCGGGACTTGGCAGCCAGAAACATCCTGGTAGCTGAGGGGCGGAA
GATGAAGATTTCGGATTTCGGCTTGTCCCGAGATGTTTATGAAGAGGATT
CCTACGTGAAGAGGAGCCAGGGTCGGATTCCAGTTAAATGGATGGCAATT
GAATCCCTTTTTGATCATATCTACACCACGCAAAGTGATGTATGGTCTTT
TGGTGTCCTGCTGTGGGAGATCGTGACCCTAGGGGGAAACCCCTATCCTG
GGATTCCTCCTGAGCGGCTCTTCAACCTTCTGAAGACCGGCCACCGGATG
GAGAGGCCAGACAACTGCAGCGAGGAGATGTACCGCCTGATGCTGCAATG
CTGGAAGCAGGAGCCGGACAAAAGGCCGGTGTTTGCGGACATCAGCAAAG
ACCTGGAGAAGATGATGGTTAAGAGGAGAGACTACTTGGACCTTGCGGCG
TCCACTCCATCTGACTCCCTGATTTATGACGACGGCCTCTCAGAGGAGGA
GACACCGCTGGTGGACTGTAATAATGCCCCCCTCCCTCGAGCCCTCCCTT
CCACATGGATTGAAAACAAACTCTATGGTAGAATTTCCCATGCATTTACT
AGATTCTAGCACCGCTGTCCCCTCTGCACTATCCTTCCTCTCTGTGATGC
TTTTTAAAAATGTTTCTGGTCTGAACAAAACCAAAGTCTGCTCTGAACCT
TTTTATTTGTAAATGTCTGACTTTGCATCCAGTTTACATTTAGGCATTAT
TGCAACTATGTTTTTCTAAAAGGAAGTGAAAATAAGTGTAATTACCACAT
TGCCCAGCAACTTAGGATGGTAGAGGAAAAAACAGATCAGGGCGGAACTC
TCAGGGGAGACCAAGAACAGGTTGAATAAGGCGCTTCTGGGGTGGGAATC
AAGTCATAGTACTTCTACTTTAACTAAGTGGATAAATATACAAATCTGGG
GAGGTATTCAGTTGAGAAAGGAGCCACCAGCACCACTCAGCCTGCACTGG
GAGCACAGCCAGGTTCCCCCAGACCCCTCCTGGGCAGGCAGGTGCCTCTC
AGAGGCCACCCGGCACTGGCGAGCAGCCACTGGCCAAGCCTCAGCCCCAG
TCCCAGCCACATGTCCTCCATCAGGGGTAGCGAGGTTGCAGGAGCTGGCT
GGCCCTGGGAGGACGCACCCCCACTGCTGTTTTCACATCCTTTCCCTTAC
CCACCTTCAGGACGGTTGTCACTTATGAAGTCAGTGCTAAAGCTGGAGCA
GTTGCTTTTTGAAAGAACATGGTCTGTGGTGCTGTGGTCTTACAATGGAC
AGTAAATATGGTTCTTGCCAAAACTCCTTCTTTTGTCTTTGATTAAATAC
TAGAAATTTAAAAAAAAAAAAAAA

In some aspects, provided herein are rearrangements involving a ROS1 gene, as well as ROS1 fusion nucleic acid molecules and polypeptides.

As used herein “c-ros oncogene 1” or “ROS1” refer to a gene encoding a ROS1 mRNA or polypeptide. The ROS1 gene encodes the ROS1 receptor tyrosine kinase protein. ROS1 is also known as ROS, MCF3, c-ros-1, c-ros oncogene 1, and ROS1 receptor tyrosine kinase. In some embodiments, a ROS1 gene is a human ROS1 gene. An exemplary ROS1 gene is represented by NCBI Gene ID No. 6098. An exemplary ROS1 mRNA sequence is represented by NCBI Ref. Seq. NM_002944, provided below as SEQ ID NO: 11. An exemplary amino acid sequence of a ROS1 polypeptide is represented by NCBI Ref. Seq. NP_002935.

(SEQ ID NO: 11)
GCACTTCTAAGAACTAACCTTTAGTCACTGGGTGACTTTATGGGAGTAAA
AGGAAGCTGTTATGAAATAGCTCTTATGGAACTGTTACAAGCTTTCAAGC
ATTCAAAGGTCTAAATGAAAAAGGCTAAGTATTATTTCAAAAGGCAAGTA
TATCCTAATATAGCAAAACAAACAAAGCAAAATCCATCAGCTACTCCTCC
AATTGAAGTGATGAAGCCCAAATAATTCATATAGCAAAATGGAGAAAATT
AGACCGGCCATCTAAAAATCTGCCATTGGTGAAGTGATGAAGAACATTTA
CTGTCTTATTCCGAAGCTTGTCAATTTTGCAACTCTTGGCTGCCTATGGA
TTTCTGTGGTGCAGTGTACAGTTTTAAATAGCTGCCTAAAGTCGTGTGTA
ACTAATCTGGGCCAGCAGCTTGACCTTGGCACACCACATAATCTGAGTGA
ACCGTGTATCCAAGGATGTCACTTTTGGAACTCTGTAGATCAGAAAAACT
GTGCTTTAAAGTGTCGGGAGTCGTGTGAGGTTGGCTGTAGCAGCGCGGAA
GGTGCATATGAAGAGGAAGTACTGGAAAATGCAGACCTACCAACTGCTCC
CTTTGCTTCTTCCATTGGAAGCCACAATATGACATTACGATGGAAATCTG
CAAACTTCTCTGGAGTAAAATACATCATTCAGTGGAAATATGCACAACTT
CTGGGAAGCTGGACTTATACTAAGACTGTGTCCAGACCGTCCTATGTGGT
CAAGCCCCTGCACCCCTTCACTGAGTACATTTTCCGAGTGGTTTGGATCT
TCACAGCGCAGCTGCAGCTCTACTCCCCTCCAAGTCCCAGTTACAGGACT
CATCCTCATGGAGTTCCTGAAACTGCACCTTTGATTAGGAATATTGAGAG
CTCAAGTCCCGACACTGTGGAAGTCAGCTGGGATCCACCTCAATTCCCAG
GTGGACCTATTTTGGGTTATAACTTAAGGCTGATCAGCAAAAATCAAAAA
TTAGATGCAGGGACACAGAGAACCAGTTTCCAGTTTTACTCCACTTTACC
AAATACTATCTACAGGTTTTCTATTGCAGCAGTAAATGAAGTTGGTGAGG
GTCCAGAAGCAGAATCTAGTATTACCACTTCATCTTCAGCAGTTCAACAA
GAGGAACAGTGGCTCTTTTTATCCAGAAAAACTTCTCTAAGAAAGAGATC
TTTAAAACATTTAGTAGATGAAGCACATTGCCTTCGGTTGGATGCTATAT
ACCATAATATTACAGGAATATCTGTTGATGTCCACCAGCAAATTGTTTAT
TTCTCTGAAGGAACTCTCATATGGGCGAAGAAGGCTGCCAACATGTCTGA
TGTATCTGACCTGAGAATTTTTTACAGAGGTTCAGGATTAATTTCTTCTA
TCTCCATAGATTGGCTTTATCAAAGAATGTATTTCATCATGGATGAACTG
GTATGTGTCTGTGATTTAGAGAACTGCTCAAACATCGAGGAAATTACTCC
ACCCTCTATTAGTGCACCTCAAAAAATTGTGGCTGATTCATACAATGGGT
ATGTCTTTTACCTCCTGAGAGATGGCATTTATAGAGCAGACCTTCCTGTA
CCATCTGGCCGGTGTGCAGAAGCTGTGCGTATTGTGGAGAGTTGCACGTT
AAAGGACTTTGCAATCAAGCCACAAGCCAAGCGAATCATTTACTTCAATG
ACACTGCCCAAGTCTTCATGTCAACATTTCTGGATGGCTCTGCTTCCCAT
CTCATCCTACCTCGCATCCCCTTTGCTGATGTGAAAAGTTTTGCTTGTGA
AAACAATGACTTTCTTGTCACAGATGGCAAGGTCATTTTCCAACAGGATG
CTTTGTCTTTTAATGAATTCATCGTGGGATGTGACCTGAGTCACATAGAA
GAATTTGGGTTTGGTAACTTGGTCATCTTTGGCTCATCCTCCCAGCTGCA
CCCTCTGCCAGGCCGCCCGCAGGAGCTTTCGGTGCTGTTTGGCTCTCACC
AGGCTCTTGTTCAATGGAAGCCTCCTGCCCTTGCCATAGGAGCCAATGTC
ATCCTGATCAGTGATATTATTGAACTCTTTGAATTAGGCCCTTCTGCCTG
GCAGAACTGGACCTATGAGGTGAAAGTATCCACCCAAGACCCTCCTGAAG
TCACTCATATTTTCTTGAACATAAGTGGAACCATGCTGAATGTACCTGAG
CTGCAGAGTGCTATGAAATACAAGGTTTCTGTGAGAGCAAGTTCTCCAAA
GAGGCCAGGCCCCTGGTCAGAGCCCTCAGTGGGTACTACCCTGGTGCCAG
CTAGTGAACCACCATTTATCATGGCTGTGAAAGAAGATGGGCTTTGGAGT
AAACCATTAAATAGCTTTGGCCCAGGAGAGTTCTTATCCTCTGATATAGG
AAATGTGTCAGACATGGATTGGTATAACAACAGCCTCTACTACAGTGACA
CGAAAGGCGACGTTTTTGTGTGGCTGCTGAATGGGACGGATATCTCAGAG
AATTATCACCTACCCAGCATTGCAGGAGCAGGGGCTTTAGCTTTTGAGTG
GCTGGGTCACTTTCTCTACTGGGCTGGAAAGACATATGTGATACAAAGGC
AGTCTGTGTTGACGGGACACACAGACATTGTTACCCACGTGAAGCTATTG
GTGAATGACATGGTGGTGGATTCAGTTGGTGGATATCTCTACTGGACCAC
ACTCTATTCAGTGGAAAGCACCAGACTAAATGGGGAAAGTTCCCTTGTAC
TACAGACACAGCCTTGGTTTTCTGGGAAAAAGGTAATTGCTCTAACTTTA
GACCTCAGTGATGGGCTCCTGTATTGGTTGGTTCAAGACAGTCAATGTAT
TCACCTGTACACAGCTGTTCTTCGGGGACAGAGCACTGGGGATACCACCA
TCACAGAATTTGCAGCCTGGAGTACTTCTGAAATTTCCCAGAATGCACTG
ATGTACTATAGTGGTCGGCTGTTCTGGATCAATGGCTTTAGGATTATCAC
AACTCAAGAAATAGGTCAGAAAACCAGTGTCTCTGTTTTGGAACCAGCCA
GATTTAATCAGTTCACAATTATTCAGACATCCCTTAAGCCCCTGCCAGGG
AACTTTTCCTTTACCCCTAAGGTTATTCCAGATTCTGTTCAAGAGTCTTC
ATTTAGGATTGAAGGAAATGCTTCAAGTTTTCAAATCCTGTGGAATGGTC
CCCCTGCGGTAGACTGGGGTGTAGTTTTCTACAGTGTAGAATTTAGTGCT
CATTCTAAGTTCTTGGCTAGTGAACAACACTCTTTACCTGTATTTACTGT
GGAAGGACTGGAACCTTATGCCTTATTTAATCTTTCTGTCACTCCTTATA
CCTACTGGGGAAAGGGCCCCAAAACATCTCTGTCACTTCGAGCACCTGAA
ACAGTTCCATCAGCACCAGAGAACCCCAGAATATTTATATTACCAAGTGG
AAAATGCTGCAACAAGAATGAAGTTGTGGTGGAATTTAGGTGGAACAAAC
CTAAGCATGAAAATGGGGTGTTAACAAAATTTGAAATTTTCTACAATATA
TCCAATCAAAGTATTACAAACAAAACATGTGAAGACTGGATTGCTGTCAA
TGTCACTCCCTCAGTGATGTCTTTTCAACTTGAAGGCATGAGTCCCAGAT
GCTTTATTGCCTTCCAGGTTAGGGCCTTTACATCTAAGGGGCCAGGACCA
TATGCTGACGTTGTAAAGTCTACAACATCAGAAATCAACCCATTTCCTCA
CCTCATAACTCTTCTTGGTAACAAGATAGTTTTTTTAGATATGGATCAAA
ATCAAGTTGTGTGGACGTTTTCAGCAGAAAGAGTTATCAGTGCCGTTTGC
TACACAGCTGATAATGAGATGGGATATTATGCTGAAGGGGACTCACTCTT
TCTTCTGCACTTGCACAATCGCTCTAGCTCTGAGCTTTTCCAAGATTCAC
TGGTTTTTGATATCACAGTTATTACAATTGACTGGATTTCAAGGCACCTC
TACTTTGCACTGAAAGAATCACAAAATGGAATGCAAGTATTTGATGTTGA
TCTTGAACACAAGGTGAAATATCCCAGAGAGGTGAAGATTCACAATAGGA
ATTCAACAATAATTTCTTTTTCTGTATATCCTCTTTTAAGTCGCTTGTAT
TGGACAGAAGTTTCCAATTTTGGCTACCAGATGTTCTACTACAGTATTAT
CAGTCACACCTTGCACCGAATTCTGCAACCCACAGCTACAAACCAACAAA
ACAAAAGGAATCAATGTTCTTGTAATGTGACTGAATTTGAGTTAAGTGGA
GCAATGGCTATTGATACCTCTAACCTAGAGAAACCATTGATATACTTTGC
CAAAGCACAAGAGATCTGGGCAATGGATCTGGAAGGCTGTCAGTGTTGGA
GAGTTATCACAGTACCTGCTATGCTCGCAGGAAAAACCCTTGTTAGCTTA
ACTGTGGATGGAGATCTTATATACTGGATCATCACAGCAAAGGACAGCAC
ACAGATTTATCAGGCAAAGAAAGGAAATGGGGCCATCGTTTCCCAGGTGA
AGGCCCTAAGGAGTAGGCATATCTTGGCTTACAGTTCAGTTATGCAGCCT
TTTCCAGATAAAGCGTTTCTGTCTCTAGCTTCAGACACTGTGGAACCAAC
TATACTTAATGCCACTAACACTAGCCTCACAATCAGATTACCTCTGGCCA
AGACAAACCTCACATGGTATGGCATCACCAGCCCTACTCCAACATACCTG
GTTTATTATGCAGAAGTTAATGACAGGAAAAACAGCTCTGACTTGAAATA
TAGAATTCTGGAATTTCAGGACAGTATAGCTCTTATTGAAGATTTACAAC
CATTTTCAACATACATGATACAGATAGCTGTAAAAAATTATTATTCAGAT
CCTTTGGAACATTTACCACCAGGAAAAGAGATTTGGGGAAAAACTAAAAA
TGGAGTACCAGAGGCAGTGCAGCTCATTAATACAACTGTGCGGTCAGACA
CCAGCCTCATTATATCTTGGAGAGAATCTCACAAGCCAAATGGACCTAAA
GAATCAGTCCGTTATCAGTTGGCAATCTCACACCTGGCCCTAATTCCTGA
AACTCCTCTAAGACAAAGTGAATTTCCAAATGGAAGGCTCACTCTCCTTG
TTACTAGACTGTCTGGTGGAAATATTTATGTGTTAAAGGTTCTTGCCTGC
CACTCTGAGGAAATGTGGTGTACAGAGAGTCATCCTGTCACTGTGGAAAT
GTTTAACACACCAGAGAAACCTTATTCCTTGGTTCCAGAGAACACTAGTT
TGCAATTTAATTGGAAGGCTCCATTGAATGTTAACCTCATCAGATTTTGG
GTTGAGCTACAGAAGTGGAAATACAATGAGTTTTACCATGTTAAAACTTC
ATGCAGCCAAGGTCCTGCTTATGTCTGTAATATCACAAATCTACAACCTT
ATACTTCATATAATGTCAGAGTAGTGGTGGTTTATAAGACGGGAGAAAAT
AGCACCTCACTTCCAGAAAGCTTTAAGACAAAAGCTGGAGTCCCAAATAA
ACCAGGCATTCCCAAATTACTAGAAGGGAGTAAAAATTCAATACAGTGGG
AGAAAGCTGAAGATAATGGATGTAGAATTACATACTATATCCTTGAGATA
AGAAAGAGCACTTCAAATAATTTACAGAACCAGAATTTAAGGTGGAAGAT
GACATTTAATGGATCCTGCAGTAGTGTTTGCACATGGAAGTCCAAAAACC
TGAAAGGAATATTTCAGTTCAGAGTAGTAGCTGCAAATAATCTAGGGTTT
GGTGAATATAGTGGAATCAGTGAGAATATTATATTAGTTGGAGATGATTT
TTGGATACCAGAAACAAGTTTCATACTTACTATTATAGTTGGAATATTTC
TGGTTGTTACAATCCCACTGACCTTTGTCTGGCATAGAAGATTAAAGAAT
CAAAAAAGTGCCAAGGAAGGGGTGACAGTGCTTATAAACGAAGACAAAGA
GTTGGCTGAGCTGCGAGGTCTGGCAGCCGGAGTAGGCCTGGCTAATGCCT
GCTATGCAATACATACTCTTCCAACCCAAGAGGAGATTGAAAATCTTCCT
GCCTTCCCTCGGGAAAAACTGACTCTGCGTCTCTTGCTGGGAAGTGGAGC
CTTTGGAGAAGTGTATGAAGGAACAGCAGTGGACATCTTAGGAGTTGGAA
GTGGAGAAATCAAAGTAGCAGTGAAGACTTTGAAGAAGGGTTCCACAGAC
CAGGAGAAGATTGAATTCCTGAAGGAGGCACATCTGATGAGCAAATTTAA
TCATCCCAACATTCTGAAGCAGCTTGGAGTTTGTCTGCTGAATGAACCCC
AATACATTATCCTGGAACTGATGGAGGGAGGAGACCTTCTTACTTATTTG
CGTAAAGCCCGGATGGCAACGTTTTATGGTCCTTTACTCACCTTGGTTGA
CCTTGTAGACCTGTGTGTAGATATTTCAAAAGGCTGTGTCTACTTGGAAC
GGATGCATTTCATTCACAGGGATCTGGCAGCTAGAAATTGCCTTGTTTCC
GTGAAAGACTATACCAGTCCACGGATAGTGAAGATTGGAGACTTTGGACT
CGCCAGAGACATCTATAAAAATGATTACTATAGAAAGAGAGGGGAAGGCC
TGCTCCCAGTTCGGTGGATGGCTCCAGAAAGTTTGATGGATGGAATCTTC
ACTACTCAATCTGATGTATGGTCTTTTGGAATTCTGATTTGGGAGATTTT
AACTCTTGGTCATCAGCCTTATCCAGCTCATTCCAACCTTGATGTGTTAA
ACTATGTGCAAACAGGAGGGAGACTGGAGCCACCAAGAAATTGTCCTGAT
GATCTGTGGAATTTAATGACCCAGTGCTGGGCTCAAGAACCCGACCAAAG
ACCTACTTTTCATAGAATTCAGGACCAACTTCAGTTATTCAGAAATTTTT
TCTTAAATAGCATTTATAAGTCCAGAGATGAAGCAAACAACAGTGGAGTC
ATAAATGAAAGCTTTGAAGGTGAAGATGGCGATGTGATTTGTTTGAATTC
AGATGACATTATGCCAGTTGCTTTAATGGAAACGAAGAACCGAGAAGGGT
TAAACTATATGGTACTTGCTACAGAATGTGGCCAAGGTGAAGAAAAGTCT
GAGGGTCCTCTAGGCTCCCAGGAATCTGAATCTTGTGGTCTGAGGAAAGA
AGAGAAGGAACCACATGCAGACAAAGATTTCTGCCAAGAAAAACAAGTGG
CTTACTGCCCTTCTGGCAAGCCTGAAGGCCTGAACTATGCCTGTCTCACT
CACAGTGGATATGGAGATGGGTCTGATTAATAGCGTTGTTTGGGAAATAG
AGAGTTGAGATAAACACTCTCATTCAGTAGTTACTGAAAGAAAACTCTGC
TAGAATGATAAATGTCATGGTGGTCTATAACTCCAAATAAACAATGCAAC
GTTCCTGATTTCTAATCTTGGTTCTGAGAGCCATTTGGTTTCAGTTGTAG
CAATCCCCATACCAGCTGCCTGACTTTCAGTAGAATTATGAGATGAACAC
TAAGCATGTGGAAAGCTTAGGAAGACTCAGAAGTCTGGAAGGGAAACACT
GCTCTCCCTTCTCCCTTGAGGTGCTTTAGGCTCTTACCCACCTTTCAGTT
TGGGCTGTAATAAAAATATCTTGGCCACATGTTTAGAGACAGAATAGGTG
TGTTCAGCGATATAAAGAAGAGGCTAAGGAGTAGGCTCAGGGGGGTCAAC
TGAACTACAGATAATCTCAAATGGGACCAAGGAAATGAGAAATAATTTCA
CACATACAGAAGAAACCAGCACCTGTGACTTGAGAAATCACTTGGAAAGC
TGTTACTGCAATGATATATATATTATCTTTTTTTAATTTTTTTTTTTTTT
TTTTGAGACGAAGTCTTGCTCTGTTGCCCAGGCTGGAGTTCAATGGCACG
ATCTCGGCACTGCAAACTCCACCTCCTGGGTTCAAGCAATTCTCGTGCCT
CAGCCTCCTAAGTAGCTGGGATTACAGGCGTGTGCCACCACGCCCGGCTA
ATTTTTGTATTTTTAGTAGAGATGAGGTTTCACCATATTGGCCAGGCTGG
TCTAGAACTCCTGACCTCGGGATCCACCTGCCTTGGCCTCCCAAAGTGCT
GGATTACAGGTGTGAGCCACCATGCCTAGCCGATATATATTGTCTTTAAT
CACTACTGTAAAATATTTTGTAGTTTTGAGGCTTACAACAGTAGATTCAG
TCATGTTGAAAATAAGACTGTGAAGATCTTTTAAGTCCTGAAGTTTTGCA
TTCTGTAATCTTCAGTTGTATAAAATCACTCTGACTTGTGTGCTATTATG
GAAATTAACTAGTATAAAGATTGCTATTTGCCATATCTATTTTATGTATA
AAATACTTAAGATTACATTTTGTATCAAATTATGCTTAAAATTAAATATA
AATGATTATACAATGTTAA

### (i) Kinase Fusion Nucleic Acid Molecules

In some embodiments, an ALK, BRAF, EGFR, ERBB2, FGFR1, FGFR2, FGFR3, MET, NTRK1, RAF1, RET, or ROS1 rearrangement results in a gene fusion, resulting in a fusion nucleic acid molecule comprising at least a portion of an ALK, BRAF, EGFR, ERBB2, FGFR1, FGFR2, FGFR3, MET, NTRK1, RAF1, RET, or ROS1 gene, and at least a portion of another gene.

In some aspects, provided herein are ALK fusion nucleic acid molecules comprising at least a portion of ALK and at least a portion of another gene.

In some embodiments, an ALK fusion nucleic acid molecule of the disclosure comprises at least a portion of ALK and at least a portion of AGAP1, ARHGEF7, BRE, EPS8, GPR113, HDAC9, MIPOL1, PELI1, SLC39A10, VKORC1L1, PLEKHA7, SPINK5, GCC2, HIP1, KANK1, KLC1, PPFIBP1, SORBS1, TFG, or TPM3. For example, in some embodiments, the ALK fusion nucleic acid molecule is selected from AGAP1-ALK, ARHGEF7-ALK, BRE-ALK, EPS8-ALK, GPR113-ALK, HDAC9-ALK, MIPOL1-ALK, PELI1-ALK, SLC39A10-ALK, VKORC1L1-ALK, ALK-SORBS1, ALK-SPINK5, GCC2-ALK, HIP1-ALK, KANK1-ALK, PLEKHA7-ALK, KLC1-ALK, TFG-ALK, TPM3-ALK, or PPFIBP1-ALK, wherein the order of the genes is in the 5′ to 3′ direction. Exemplary and non-limiting ALK fusion nucleic acid molecules are described herein and/or in any of Tables 1-6, and/or in the Examples herein.

As used herein “AGAP1” refers to a gene encoding an AGAP1 mRNA or polypeptide. The AGAP1 gene encodes the Arf-GAP with GTPase, ANK repeat and PH domain-containing protein 1. AGAP1 is also known as CENTG2 and KIAA1099. In some embodiments, an AGAP1 gene is a human AGAP1 gene. An exemplary AGAP1 gene is represented by NCBI Gene ID No. 116987. An exemplary AGAP1 mRNA sequence is represented by NCBI Ref. Seq. NM_014914. An exemplary amino acid sequence of an AGAP1 polypeptide is represented by NCBI Ref. Seq. NP_055729.

As used herein “ARHGEF7” refers to a gene encoding an ARHGEF7 mRNA or polypeptide. The ARHGEF7 gene encodes the Rho Guanine Nucleotide Exchange Factor 7 protein. ARHGEF7 is also known as P50, P85, PAK3, PIXB, COOL1, P50BP, COOL-1, P85SPR, BETA-PIX, P85COOL1, and Nbla10314. In some embodiments, an ARHGEF7 gene is a human ARHGEF7 gene. An exemplary ARHGEF7 gene is represented by NCBI Gene ID No. 8874. An exemplary ARHGEF7 mRNA sequence is represented by NCBI Ref. Seq. NM_145735. An exemplary amino acid sequence of an ARHGEF7 polypeptide is represented by NCBI Ref. Seq. NP_663788.

As used herein “BRE” refers to a gene encoding a BRE mRNA or polypeptide. The BRE gene encodes the brain and reproductive organ-expressed protein. BRE is also known as BABAM2, BRCC4, and BRCC45. In some embodiments, a BRE gene is a human BRE gene. An exemplary BRE gene is represented by NCBI Gene ID No. 9577. An exemplary BRE mRNA sequence is represented by NCBI Ref. Seq. NM_004899. An exemplary amino acid sequence of a BRE polypeptide is represented by NCBI Ref. Seq. NP_004890.

As used herein “EPS8” refers to a gene encoding an EPS8 mRNA or polypeptide. The EPS8 gene encodes the epidermal growth factor receptor pathway substrate 8 protein. EPS8 is also known as DFNB102. In some embodiments, an EPS8 gene is a human EPS8 gene. An exemplary EPS8 gene is represented by NCBI Gene ID No. 2059. An exemplary EPS8 mRNA sequence is represented by NCBI Ref. Seq. NM_004447. An exemplary amino acid sequence of an EPS8 polypeptide is represented by NCBI Ref. Seq. NP_004438.

As used herein “GPR113” refers to a gene encoding a GPR113 mRNA or polypeptide. The GPR113 gene encodes the G-protein coupled receptor 113 protein. GPR113 is also known as ADGRF3 and PGR23. In some embodiments, a GPR113 gene is a human GPR113 gene. An exemplary GPR113 gene is represented by NCBI Gene ID No. 165082. An exemplary GPR113 mRNA sequence is represented by NCBI Ref. Seq. NM_153835. An exemplary amino acid sequence of a GPR113 polypeptide is represented by NCBI Ref. Seq. NP_722577.

As used herein “HDAC9” refers to a gene encoding an HDAC9 mRNA or polypeptide. The HDAC9 gene encodes the histone deacetylase 9 protein. HDAC9 is also known as HD7, HD9, HD7b, HDAC, HDRP, MITR, HDAC7, HDAC7B, HDAC9B, and HDAC9FL. In some embodiments, an HDAC9 gene is a human HDAC9 gene. An exemplary HDAC9 gene is represented by NCBI Gene ID No. 9734. An exemplary HDAC9 mRNA sequence is represented by NCBI Ref. Seq. NM_058176. An exemplary amino acid sequence of an HDAC9 polypeptide is represented by NCBI Ref. Seq. NP_478056.

As used herein “MIPOL1” refers to a gene encoding a MIPOL1 mRNA or polypeptide. The MIPOL1 gene encodes the mirror-image polydactyly 1 protein. MIPOL1 is also known as CCDC193. In some embodiments, a MIPOL1 gene is a human MIPOL1 gene. An exemplary MIPOL1 gene is represented by NCBI Gene ID No. 145282. An exemplary MIPOL1 mRNA sequence is represented by NCBI Ref. Seq. NM_138731. An exemplary amino acid sequence of a MIPOL1 polypeptide is represented by NCBI Ref. Seq. NP_620059.

As used herein “PELI1” refers to a gene encoding a PELI1 mRNA or polypeptide. The PELI1 gene encodes the mirror-image polydactyly 1 protein. In some embodiments, a PELI1 gene is a human PELI1 gene. An exemplary PELI1 gene is represented by NCBI Gene ID No. 57162. An exemplary PELI1 mRNA sequence is represented by NCBI Ref. Seq. NM_020651. An exemplary amino acid sequence of a PELI1 polypeptide is represented by NCBI Ref. Seq. NP_065702.

As used herein “SLC39A10” refers to a gene encoding a SLC39A10 mRNA or polypeptide. The SLC39A10 gene encodes the solute carrier family 39 member 10 protein. SLC39A10 is also known as LZT-Hs2. In some embodiments, an SLC39A10 gene is a human SLC39A10 gene. An exemplary SLC39A10 gene is represented by NCBI Gene ID No. 57181. An exemplary SLC39A10 mRNA sequence is represented by NCBI Ref. Seq. NM_020342. An exemplary amino acid sequence of an SLC39A10 polypeptide is represented by NCBI Ref. Seq. NP_065075.

As used herein “VKORCIL1” refers to a gene encoding a VKORC1L1 mRNA or polypeptide. The VKORC1L1 gene encodes the vitamin K epoxide reductase complex subunit 1 like 1 protein. In some embodiments, a VKORC1L1 gene is a human VKORC1L1 gene. An exemplary VKORC1L1 gene is represented by NCBI Gene ID No. 154807. An exemplary VKORC1L1 mRNA sequence is represented by NCBI Ref. Seq. NM_173517. An exemplary amino acid sequence of a VKORC1L1 polypeptide is represented by NCBI Ref. Seq. NP_775788.

As used herein “SORBS1” refers to a gene encoding a SORBS1 mRNA or polypeptide. The SORBS1 gene encodes the sorbin and SH3 domain containing 1 protein. SORBS1 is also known as CAP, FLAF2, R85FL, SH3D5, SORB1, and SH3P12. In some embodiments, a SORBS1 gene is a human SORBS1 gene. An exemplary SORBS1 gene is represented by NCBI Gene ID No. 10580. An exemplary SORBS1 mRNA sequence is represented by NCBI Ref. Seq. NM_006434. An exemplary amino acid sequence of a SORBS1 polypeptide is represented by NCBI Ref. Seq. NP_006425.

As used herein “SPINK5” refers to a gene encoding a SPINK5 mRNA or polypeptide. The SPINK5 gene encodes the serine peptidase inhibitor Kazal type 5 protein. SPINK5 is also known as NS, NETS, LEKTI, LETKI, and VAKTI. In some embodiments, a SPINK5 gene is a human SPINK5 gene. An exemplary SPINK5 gene is represented by NCBI Gene ID No. 11005. An exemplary SPINK5 mRNA sequence is represented by NCBI Ref. Seq. NM_006846. An exemplary amino acid sequence of a SPINK5 polypeptide is represented by NCBI Ref. Seq. NP_006837.

As used herein “GCC2” refers to a gene encoding a GCC2 mRNA or polypeptide. The GCC2 gene encodes the GRIP and coiled-coil domain containing 2 protein. GCC2 is also known as REN53, GCC185, and RANBP2L4. In some embodiments, a GCC2 gene is a human GCC2 gene. An exemplary GCC2 gene is represented by NCBI Gene ID No. 9648. An exemplary GCC2 mRNA sequence is represented by NCBI Ref. Seq. NM_181453. An exemplary amino acid sequence of a GCC2 polypeptide is represented by NCBI Ref. Seq. NP_852118.

As used herein “HIP1” refers to a gene encoding a HIP1 mRNA or polypeptide. The HIP1 gene encodes the huntingtin interacting protein 1 protein. HIP1 is also known as SHON, HIP-I, ILWEQ, SHONbeta, and SHONgamma. In some embodiments, a HIP1 gene is a human HIP1 gene. An exemplary HIP1 gene is represented by NCBI Gene ID No. 3092. An exemplary HIP1 mRNA sequence is represented by NCBI Ref. Seq. NM_005338. An exemplary amino acid sequence of a HIP1 polypeptide is represented by NCBI Ref. Seq. NP_005329.

As used herein “KANK1” refers to a gene encoding a KANK1 mRNA or polypeptide. The KANK1 gene encodes the KN motif and ankyrin repeat domains 1 protein. KANK1 is also known as KANK, CPSQ2, and ANKRD15. In some embodiments, a KANK1 gene is a human KANK1 gene. An exemplary KANK1 gene is represented by NCBI Gene ID No. 23189. An exemplary KANK1 mRNA sequence is represented by NCBI Ref. Seq. NM_015158. An exemplary amino acid sequence of a KANK1 polypeptide is represented by NCBI Ref. Seq. NP_055973.

As used herein “PLEKHA7” refers to a gene encoding a PLEKHA7 mRNA or polypeptide. The PLEKHA7 gene encodes the pleckstrin homology domain containing A7 protein. PLEKHA7 is also known as DKFZp686M22243. In some embodiments, a PLEKHA7 gene is a human PLEKHA7 gene. An exemplary PLEKHA7 gene is represented by NCBI Gene ID No. 144100. An exemplary PLEKHA7 mRNA sequence is represented by NCBI Ref. Seq. NM_001329630. An exemplary amino acid sequence of a PLEKHA7 polypeptide is represented by NCBI Ref. Seq. NP_001316559.

As used herein “KLC1” refers to a gene encoding a KLC1 mRNA or polypeptide. The KLC1 gene encodes the kinesin light chain 1 protein. KLC1 is also known as KLC, KNS2, and KNS2A. In some embodiments, a KLC1 gene is a human KLC1 gene. An exemplary KLC1 gene is represented by NCBI Gene ID No. 3831. An exemplary KLC1 mRNA sequence is represented by NCBI Ref. Seq. NM_005552. An exemplary amino acid sequence of a KLC1 polypeptide is represented by NCBI Ref. Seq. NP_005543.

As used herein “TFG” refers to a gene encoding a TFG mRNA or polypeptide. The TFG gene encodes the trafficking from ER to golgi regulator protein. TFG is also known as TF6, HMSNP, SPG57, and TRKT3. In some embodiments, a TFG gene is a human TFG gene. An exemplary TFG gene is represented by NCBI Gene ID No. 10342. An exemplary TFG mRNA sequence is represented by NCBI Ref. Seq. NM_006070. An exemplary amino acid sequence of a TFG polypeptide is represented by NCBI Ref. Seq. NP_006061.

As used herein “TPM3” refers to a gene encoding a TPM3 mRNA or polypeptide. The TPM3 gene encodes tropomyosin 3 protein. TPM3 is also known as TM3, TM5, TRK, CFTD, NEM1, TM-5, TM30, CAPM1, TM30 nm, TPM3nu, TPMsk3, hscp30, HEL-189, HEL-S-82p, and OK/SW-cl.5. In some embodiments, a TPM3 gene is a human TPM3 gene. An exemplary TPM3 gene is represented by NCBI Gene ID No. 7170. An exemplary TPM3 mRNA sequence is represented by NCBI Ref. Seq. NM_152263. An exemplary amino acid sequence of a TPM3 polypeptide is represented by NCBI Ref. Seq. NP_689476.

As used herein “PPFIBP1” refers to a gene encoding a PPFIBP1 mRNA or polypeptide. The PPFIBP1 gene encodes PPFIA binding protein 1 protein. PPFIBP1 is also known as L2, SGT2, hSGT2, and hSgt2p. In some embodiments, a PPFIBP1 gene is a human PPFIBP1 gene. An exemplary PPFIBP1 gene is represented by NCBI Gene ID No. 8496. An exemplary PPFIBP1 mRNA sequence is represented by NCBI Ref. Seq. NM_003622. An exemplary amino acid sequence of a PPFIBP1 polypeptide is represented by NCBI Ref. Seq. NP_003613.

In some aspects, provided herein are BRAF fusion nucleic acid molecules comprising at least a portion of BRAF and at least a portion of another gene.

In some embodiments, a BRAF fusion nucleic acid molecule comprises at least a portion of BRAF and at least a portion of CCDC88C, COBLL1, CREB3L2, DLC1, GOLGA3, MS12, TNS3, DOCK4, RAD51, AKAP9, ARMC10, DENND2A, JHDM1D, KIAA1549, MKRN1, NRF1, SLC45A3, SND1, ZC3HAV1, ZNF277, or TRIM24. For example, in some embodiments, the BRAF fusion nucleic acid molecule is selected from CCDC88C-BRAF, COBLL1-BRAF, CREB3L2-BRAF, DLC1-BRAF, GOLGA3-BRAF, MSI2-BRAF, TNS3-BRAF, BRAF-DOCK4, BRAF-RAD51, AKAP9-BRAF, ARMC10-BRAF, DENND2A-BRAF, JHDM1D-BRAF, KIAA1549-BRAF, MKRN1-BRAF, NRF1-BRAF, SLC45A3-BRAF, SND1-BRAF, BRAF-TRIM24, ZC3HAV1-BRAF, or ZNF277-BRAF, wherein the order of the genes is in the 5′ to 3′ direction. Exemplary and non-limiting BRAF fusion nucleic acid molecules are described herein and/or in any of Tables 1-6, and/or in the Examples herein.

As used herein “CCDC88C” refers to a gene encoding a CCDC88C mRNA or polypeptide. The CCDC88C gene encodes coiled-coil domain containing 88C protein. CCDC88C is also known as HYC1, DAPLE, HKRP2, SCA40, and KIAA1509. In some embodiments, a CCDC88C gene is a human CCDC88C gene. An exemplary CCDC88C gene is represented by NCBI Gene ID No. 440193. An exemplary CCDC88C mRNA sequence is represented by NCBI Ref. Seq. NM_001080414. An exemplary amino acid sequence of a CCDC88C polypeptide is represented by NCBI Ref. Seq. NP_001073883.

As used herein “COBLL1” refers to a gene encoding a COBLL1 mRNA or polypeptide. The COBLL1 gene encodes cordon-bleu WH2 repeat protein like 1 protein. COBLL1 is also known as COBLR1 and KIAA0977. In some embodiments, a COBLL1 gene is a human COBLL1 gene. An exemplary COBLL1 gene is represented by NCBI Gene ID No. 22837. An exemplary COBLL1 mRNA sequence is represented by NCBI Ref. Seq. NM_014900. An exemplary amino acid sequence of a COBLL1 polypeptide is represented by NCBI Ref. Seq. NP_055715.

As used herein “CREB3L2” refers to a gene encoding a CREB3L2 mRNA or polypeptide. The CREB3L2 gene encodes the cAMP responsive element binding protein 3 like 2 protein. CREB3L2 is also known as BBF2H7 and TCAG_1951439. In some embodiments, a CREB3L2 gene is a human CREB3L2 gene. An exemplary CREB3L2 gene is represented by NCBI Gene ID No. 64764. An exemplary CREB3L2 mRNA sequence is represented by NCBI Ref. Seq. NM_194071. An exemplary amino acid sequence of a CREB3L2 polypeptide is represented by NCBI Ref. Seq. NP_919047.

As used herein “DLC1” refers to a gene encoding a DLC1 mRNA or polypeptide. The DLC1 gene encodes the DLC1 Rho GTPase activating protein. DLC1 is also known as HP, ARHGAP7, STARD12, and p122-RhoGAP. In some embodiments, a DLC1 gene is a human DLC1 gene. An exemplary DLC1 gene is represented by NCBI Gene ID No. 10395. An exemplary DLC1 mRNA sequence is represented by NCBI Ref. Seq. NM_024767. An exemplary amino acid sequence of a DLC1 polypeptide is represented by NCBI Ref. Seq. NP_079043.

As used herein “GOLGA3” refers to a gene encoding a GOLGA3 mRNA or polypeptide. The GOLGA3 gene encodes the golgin A3 protein. GOLGA3 is also known as MEA-2 and GCP170. In some embodiments, a GOLGA3 gene is a human GOLGA3 gene. An exemplary GOLGA3 gene is represented by NCBI Gene ID No. 2802. An exemplary GOLGA3 mRNA sequence is represented by NCBI Ref. Seq. NM_005895. An exemplary amino acid sequence of a GOLGA3 polypeptide is represented by NCBI Ref. Seq. NP_005886.

As used herein “MS12” refers to a gene encoding a MS12 mRNA or polypeptide. The MS12 gene encodes the musashi RNA binding protein 2 protein. MS12 is also known as MSI2H. In some embodiments, a MS12 gene is a human MS12 gene. An exemplary MS12 gene is represented by NCBI Gene ID No. 124540. An exemplary MS12 mRNA sequence is represented by NCBI Ref. Seq. NM_138962. An exemplary amino acid sequence of a MS12 polypeptide is represented by NCBI Ref. Seq. NP_620412.

As used herein “TNS3” refers to a gene encoding a TNS3 mRNA or polypeptide. The TNS3 gene encodes the tensin 3 protein. TNS3 is also known as TEM6, H_NH0549123.2, FLJ13732, and TENS1. In some embodiments, a TNS3 gene is a human TNS3 gene. An exemplary TNS3 gene is represented by NCBI Gene ID No. 64759. An exemplary TNS3 mRNA sequence is represented by NCBI Ref. Seq. NM_022748. An exemplary amino acid sequence of a TNS3 polypeptide is represented by NCBI Ref. Seq. NP_073585.

As used herein “DOCK4” refers to a gene encoding a DOCK4 mRNA or polypeptide. The DOCK4 gene encodes the dedicator of cytokinesis 4 protein. DOCK4 is also known as FLJ34238 and KIAA0716. In some embodiments, a DOCK4 gene is a human DOCK4 gene. An exemplary DOCK4 gene is represented by NCBI Gene ID No. 9732. An exemplary DOCK4 mRNA sequence is represented by NCBI Ref. Seq. NM_014705. An exemplary amino acid sequence of a DOCK4 polypeptide is represented by NCBI Ref. Seq. NP_055520.

As used herein “RAD51” refers to a gene encoding a RAD51 mRNA or polypeptide. The RAD51 gene encodes the RAD51 recombinase protein. RAD51 is also known as RECA, BRCC5, FANCR, MRMV2, HRAD51, RAD51A, HsRad51, and HsT16930. In some embodiments, a RAD51 gene is a human RAD51 gene. An exemplary RAD51 gene is represented by NCBI Gene ID No. 5888. An exemplary RAD51 mRNA sequence is represented by NCBI Ref. Seq. NM_002875. An exemplary amino acid sequence of a RAD51 polypeptide is represented by NCBI Ref. Seq. NP_002866.

As used herein “AKAP9” refers to a gene encoding an AKAP9 mRNA or polypeptide. The AKAP9 gene encodes the A-kinase anchoring protein 9 protein. AKAP9 is also known as LQT11, PRKA9, AKAP-9, CG-NAP, YOTIAO, AKAP350, AKAP450, PPP1R45, HYPERION, and MU-RMS-40.16A. In some embodiments, an AKAP9 gene is a human AKAP9 gene. An exemplary AKAP9 gene is represented by NCBI Gene ID No. 10142. An exemplary AKAP9 mRNA sequence is represented by NCBI Ref. Seq. NM_005751. An exemplary amino acid sequence of an AKAP9 polypeptide is represented by NCBI Ref. Seq. NP_005742.

As used herein “ARMC10” refers to a gene encoding an ARMC10 mRNA or polypeptide. The ARMC10 gene encodes the armadillo repeat containing 10 protein. ARMC10 is also known as SVH, PNAS 112, PNAS-112, and PSEC0198. In some embodiments, an ARMC10 gene is a human ARMC10 gene. An exemplary ARMC10 gene is represented by NCBI Gene ID No. 83787. An exemplary ARMC10 mRNA sequence is represented by NCBI Ref. Seq. NM_031905. An exemplary amino acid sequence of an ARMC10 polypeptide is represented by NCBI Ref. Seq. NP_114111.

As used herein “DENND2A” refers to a gene encoding a DENND2A mRNA or polypeptide. The DENND2A gene encodes the DENN domain containing 2A protein. DENND2A is also known as FAM31D and KIAA1277. In some embodiments, a DENND2A gene is a human DENND2A gene. An exemplary DENND2A gene is represented by NCBI Gene ID No. 27147. An exemplary DENND2A mRNA sequence is represented by NCBI Ref. Seq. NM_015689. An exemplary amino acid sequence of a DENND2A polypeptide is represented by NCBI Ref. Seq. NP_056504.

As used herein “JHDM1D” refers to a gene encoding a JHDM1D mRNA or polypeptide. The JHDM1D gene encodes the jumonji C domain containing histone demethylase 1 homolog D protein. JHDM1D is also known as KDM7A. In some embodiments, a JHDM1D gene is a human JHDM1D gene. An exemplary JHDM1D gene is represented by NCBI Gene ID No. 80853. An exemplary JHDM1D mRNA sequence is represented by NCBI Ref. Seq. NM_030647. An exemplary amino acid sequence of a JHDM1D polypeptide is represented by NCBI Ref. Seq. NP_085150.

As used herein “KIAA1549” refers to a gene encoding a KIAA1549 mRNA or polypeptide. The KIAA1549 gene encodes the KIAA1549 protein. KIAA1549 is also known as RP86. In some embodiments, a KIAA1549 gene is a human KIAA1549 gene. An exemplary KIAA1549 gene is represented by NCBI Gene ID No. 57670. An exemplary KIAA1549 mRNA sequence is represented by NCBI Ref. Seq. NM_020910. An exemplary amino acid sequence of a KIAA1549 polypeptide is represented by NCBI Ref. Seq. NP_065961.

As used herein “MKRN1” refers to a gene encoding a MKRN1 mRNA or polypeptide. The MKRN1 gene encodes the makorin ring finger protein 1 protein. MKRN1 is also known as RNF61. In some embodiments, a MKRN1 gene is a human MKRN1 gene. An exemplary MKRN1 gene is represented by NCBI Gene ID No. 23608. An exemplary MKRN1 mRNA sequence is represented by NCBI Ref. Seq. NM_013446. An exemplary amino acid sequence of a MKRN1 polypeptide is represented by NCBI Ref. Seq. NP_038474.

As used herein “NRF1” refers to a gene encoding a NRF1 mRNA or polypeptide. The NRF1 gene encodes the nuclear respiratory factor 1 protein. NRF1 is also known as ALPHA-PAL and EWG. In some embodiments, a NRF1 gene is a human NRF1 gene. An exemplary NRF1 gene is represented by NCBI Gene ID No. 4899. An exemplary NRF1 mRNA sequence is represented by NCBI Ref. Seq. NM_005011. An exemplary amino acid sequence of a NRF1 polypeptide is represented by NCBI Ref. Seq. NP_005002.

As used herein “SLC45A3” refers to a gene encoding a SLC45A3 mRNA or polypeptide. The SLC45A3 gene encodes the solute carrier family 45 member 3 protein. SLC45A3 is also known as PRST, IPCA6, IPCA-2, IPCA-6, IPCA-8, PCANAP2, PCANAP6, and PCANAP8. In some embodiments, a SLC45A3 gene is a human SLC45A3 gene. An exemplary SLC45A3 gene is represented by NCBI Gene ID No. 85414. An exemplary SLC45A3 mRNA sequence is represented by NCBI Ref. Seq. NM_033102. An exemplary amino acid sequence of a SLC45A3 polypeptide is represented by NCBI Ref. Seq. NP_149093.

As used herein “SND1” refers to a gene encoding a SND1 mRNA or polypeptide. The SND1 gene encodes the staphylococcal nuclease and tudor domain containing 1 protein. SND1 is also known as p100, TDRD11, p100 EBNA2 co-activator, and Tudor-SN. In some embodiments, a SND1 gene is a human SND1 gene. An exemplary SND1 gene is represented by NCBI Gene ID No. 27044. An exemplary SND1 mRNA sequence is represented by NCBI Ref. Seq. NM_014390. An exemplary amino acid sequence of a SND1 polypeptide is represented by NCBI Ref. Seq. NP_055205.

As used herein “TRIM24” refers to a gene encoding a TRIM24 mRNA or polypeptide. The TRIM24 gene encodes the tripartite motif containing 24 protein. TRIM24 is also known as PTC6, TF1A, TIF1, RNF82, TIF1A, hTIF1, and TIF1ALPHA. In some embodiments, a TRIM24 gene is a human TRIM24 gene. An exemplary TRIM24 gene is represented by NCBI Gene ID No. 8805. An exemplary TRIM24 mRNA sequence is represented by NCBI Ref. Seq. NM_003852. An exemplary amino acid sequence of a TRIM24 polypeptide is represented by NCBI Ref. Seq. NP_003843.

As used herein “ZC3HAV1” refers to a gene encoding a ZC3HAV1 mRNA or polypeptide. The ZC3HAV1 gene encodes the zinc finger CCCH-type containing, antiviral 1 protein. ZC3HAV1 is also known as ZAP, ZC3H2, ARTD13, PARP13, FLB6421, and ZC3HDC2. In some embodiments, a ZC3HAV1 gene is a human ZC3HAV1 gene. An exemplary ZC3HAV1 gene is represented by NCBI Gene ID No. 56829. An exemplary ZC3HAV1 mRNA sequence is represented by NCBI Ref. Seq. NM_020119. An exemplary amino acid sequence of a ZC3HAV1 polypeptide is represented by NCBI Ref. Seq. NP_064504.

As used herein “ZNF277” refers to a gene encoding a ZNF277 mRNA or polypeptide. The ZNF277 gene encodes the zinc finger protein 277 protein. ZNF277 is also known as NRIF4 and ZNF277P. In some embodiments, a ZNF277 gene is a human ZNF277 gene. An exemplary ZNF277 gene is represented by NCBI Gene ID No. 11179. An exemplary ZNF277 mRNA sequence is represented by NCBI Ref. Seq. NM_021994. An exemplary amino acid sequence of a ZNF277 polypeptide is represented by NCBI Ref. Seq. NP_068834.

In some aspects, provided herein are EGFR fusion nucleic acid molecules comprising at least a portion of EGFR and at least a portion of another gene.

In some embodiments, an EGFR fusion nucleic acid molecule comprises at least a portion of EGFR and at least a portion of ABCB1, PDE7A, EZH2, FLJ45974, or ZNF479. For example, in some embodiments, the EGFR fusion nucleic acid molecule is selected from ABCB1-EGFR, PDE7A-EGFR, EGFR-EZH2, EGFR-FLJ45974, or EGFR-ZNF479, wherein the order of the genes is in the 5′ to 3′ direction. Exemplary and non-limiting EGFR fusion nucleic acid molecules are described herein and/or in Tables 1 and 3-5, and/or in the Examples herein.

As used herein “ABCB1” refers to a gene encoding an ABCB1 mRNA or polypeptide. The ABCB1 gene encodes the ATP binding cassette subfamily B member 17 protein. ABCB1 is also known as CLCS, MDR1, P-GP, PGY1, ABC20, CD243, GP170, and p-170. In some embodiments, an ABCB1 gene is a human ABCB1 gene. An exemplary ABCB1 gene is represented by NCBI Gene ID No. 5243. An exemplary ABCB1 mRNA sequence is represented by NCBI Ref. Seq. NM_000927. An exemplary amino acid sequence of an ABCB1 polypeptide is represented by NCBI Ref. Seq. NP_000918.

As used herein “PDE7A” refers to a gene encoding a PDE7A mRNA or polypeptide. The PDE7A gene encodes the phosphodiesterase 7A protein. PDE7A is also known as HCP1 and PDE7. In some embodiments, a PDE7A gene is a human PDE7A gene. An exemplary PDE7A gene is represented by NCBI Gene ID No. 5150. An exemplary PDE7A mRNA sequence is represented by NCBI Ref. Seq. NM_002603. An exemplary amino acid sequence of a PDE7A polypeptide is represented by NCBI Ref. Seq. NP_002594.

As used herein “EZH2” refers to a gene encoding an EZH2 mRNA or polypeptide. The EZH2 gene encodes the enhancer of zeste 2 polycomb repressive complex 2 subunit protein. EZH2 is also known as EZH1, WVS, ENX1, KMT6, WVS2, ENX-1, EZH2b, and KMT6A. In some embodiments, an EZH2 gene is a human EZH2 gene. An exemplary EZH2 gene is represented by NCBI Gene ID No. 2146. An exemplary EZH2 mRNA sequence is represented by NCBI Ref. Seq. NM_004456. An exemplary amino acid sequence of an EZH2 polypeptide is represented by NCBI Ref. Seq. NP_004447.

As used herein “FLJ45974” refers to a gene encoding a FLJ45974 ncRNA. The FLJ45974 gene encodes the long intergenic non-protein coding RNA 1446. FLJ45974 is also known as LINC01446. In some embodiments, an FLJ45974 gene is a human FLJ45974 gene. An exemplary FLJ45974 gene is represented by NCBI Gene ID No. 401337. An exemplary FLJ45974 ncRNA sequence is represented by NCBI Ref. Seq. NR_038371.

As used herein “ZNF479” refers to a gene encoding a ZNF479 mRNA or polypeptide. The ZNF479 gene encodes the zinc finger protein 479 protein. ZNF479 is also known as KR19 and HKr19. In some embodiments, a ZNF479 gene is a human ZNF479 gene. An exemplary ZNF479 gene is represented by NCBI Gene ID No. 90827. An exemplary ZNF479 mRNA sequence is represented by NCBI Ref. Seq. NM_033273. An exemplary amino acid sequence of a ZNF479 polypeptide is represented by NCBI Ref. Seq. NP_150376.

In some aspects, provided herein are ERBB2 fusion nucleic acid molecules comprising at least a portion of ERBB2 and at least a portion of another gene.

In some embodiments, an ERBB2 fusion nucleic acid molecule comprises at least a portion of ERBB2 and at least a portion of FBXL20, GRB7, MS12, RANBP10, SEC14L1, WIPF2, PRKCA, or PPP1R1B. For example, in some embodiments, the ERBB2 fusion nucleic acid molecule is selected from FBXL20-ERBB2, GRB7-ERBB2, MSI2-ERBB2, RANBP10-ERBB2, SEC14L1-ERBB2, WIPF2-ERBB2, ERBB2-GRB7, ERBB2-PRKCA, or ERBB2-PPP1R1B, wherein the order of the genes is in the 5′ to 3′ direction. Exemplary and non-limiting ERBB2 fusion nucleic acid molecules are described herein and/or in Tables 1-6, and/or in the Examples herein.

As used herein “FBXL20” refers to a gene encoding a FBXL20 mRNA or polypeptide. The FBXL20 gene encodes the F-box and leucine rich repeat protein 20 protein. FBXL20 is also known as Fbl2 and Fbl20. In some embodiments, a FBXL20 gene is a human FBXL20 gene. An exemplary FBXL20 gene is represented by NCBI Gene ID No. 84961. An exemplary FBXL20 mRNA sequence is represented by NCBI Ref. Seq. NM_032875. An exemplary amino acid sequence of a FBXL20 polypeptide is represented by NCBI Ref. Seq. NP_116264.

As used herein “MSI2” refers to a gene encoding a MSI2 mRNA or polypeptide. The MSI2 gene encodes the musashi RNA binding protein 2 protein. MSI2 is also known as MSI2H. In some embodiments, a MSI2 gene is a human MSI2 gene. An exemplary MSI2 gene is represented by NCBI Gene ID No. 124540. An exemplary MSI2 mRNA sequence is represented by NCBI Ref. Seq. NM_138962. An exemplary amino acid sequence of a MSI2 polypeptide is represented by NCBI Ref. Seq. NP_620412.

As used herein “RANBP10” refers to a gene encoding a RANBP10 mRNA or polypeptide. The RANBP10 gene encodes the RAN binding protein 10 protein. RANBP10 is also known as KIAA1464. In some embodiments, a RANBP10 gene is a human RANBP10 gene. An exemplary RANBP10 gene is represented by NCBI Gene ID No. 57610. An exemplary RANBP10 mRNA sequence is represented by NCBI Ref. Seq. NM_020850. An exemplary amino acid sequence of a RANBP10 polypeptide is represented by NCBI Ref. Seq. NP_065901.

As used herein “SEC14L1” refers to a gene encoding a SEC14L1 mRNA or polypeptide. The SEC14L1 gene encodes the SEC14 like lipid binding 1 protein. SEC14L1 is also known as SEC14L and PRELID4A. In some embodiments, a SEC14L1 gene is a human SEC14L1 gene. An exemplary SEC14L1 gene is represented by NCBI Gene ID No. 6397. An exemplary SEC14L1 mRNA sequence is represented by NCBI Ref. Seq. NM_003003. An exemplary amino acid sequence of a SEC14L1 polypeptide is represented by NCBI Ref. Seq. NP_002994.

As used herein “WIPF2” refers to a gene encoding a WIPF2 mRNA or polypeptide. The WIPF2 gene encodes the WAS/WASL interacting protein family member 2 protein. WIPF2 is also known as WICH and WIRE. In some embodiments, a WIPF2 gene is a human WIPF2 gene. An exemplary WIPF2 gene is represented by NCBI Gene ID No. 147179. An exemplary WIPF2 mRNA sequence is represented by NCBI Ref. Seq. NM_133264. An exemplary amino acid sequence of a WIPF2 polypeptide is represented by NCBI Ref. Seq. NP_57357.

As used herein “GRB7” refers to a gene encoding a GRB7 mRNA or polypeptide. The GRB7 gene encodes the growth factor receptor bound protein 7 protein. In some embodiments, a GRB7 gene is a human GRB7 gene. An exemplary GRB7 gene is represented by NCBI Gene ID No. 2886. An exemplary GRB7 mRNA sequence is represented by NCBI Ref. Seq. NM_005310. An exemplary amino acid sequence of a GRB7 polypeptide is represented by NCBI Ref. Seq. NP_005301.

As used herein “PRKCA” refers to a gene encoding a PRKCA mRNA or polypeptide. The PRKCA gene encodes the protein kinase C alpha protein. PRKCA is also known as AAG6, PKCA, PRKACA, PKCI+/−, PKCα, and PKC-α. In some embodiments, a PRKCA gene is a human PRKCA gene. An exemplary PRKCA gene is represented by NCBI Gene ID No. 5578. An exemplary PRKCA mRNA sequence is represented by NCBI Ref. Seq. NM_002737. An exemplary amino acid sequence of a PRKCA polypeptide is represented by NCBI Ref. Seq. NP_002728.

As used herein “PPP1R1B” refers to a gene encoding a PPP1R1B mRNA or polypeptide. The PPP1R1B gene encodes the protein phosphatase 1 regulatory inhibitor subunit 1B protein. PPP1R1B is also known as DARPP32, DARPP-32, and FLJ20940. In some embodiments, PPP1R1B gene is a human PPP1R1B gene. An exemplary PPP1R1B gene is represented by NCBI Gene ID No. 84152. An exemplary PPP1R1B mRNA sequence is represented by NCBI Ref. Seq. NM_032192. An exemplary amino acid sequence of a PPP1R1B polypeptide is represented by NCBI Ref. Seq. NP_115568.

In some aspects, provided herein are FGFR1 fusion nucleic acid molecules comprising at least a portion of FGFR1 and at least a portion of another gene.

In some embodiments, an FGFR1 fusion nucleic acid molecule comprises at least a portion of FGFR1 and at least a portion of ADAM32, SLC12A8, ADAM18, BAG4, or TACC1. For example, in some embodiments, the FGFR1 fusion nucleic acid molecule is selected from FGFR1-ADAM32, FGFR1-SLC12A8, ADAM18-FGFR1, BAG4-FGFR1, or FGFR1-TACC1, wherein the order of the genes is in the 5′ to 3′ direction. Exemplary and non-limiting FGFR1 fusion nucleic acid molecules are described herein and/or in Tables 1-6, and/or in the Examples herein.

As used herein “ADAM32” refers to a gene encoding an ADAM32 mRNA or polypeptide. The ADAM32 gene encodes the ADAM metallopeptidase domain 32 protein. In some embodiments, ADAM32 gene is a human ADAM32 gene. An exemplary ADAM32 gene is represented by NCBI Gene ID No. 203102. An exemplary ADAM32 mRNA sequence is represented by NCBI Ref. Seq. NM_145004. An exemplary amino acid sequence of an ADAM32 polypeptide is represented by NCBI Ref. Seq. NP_659441.

As used herein “SLC12A8” refers to a gene encoding an SLC12A8 mRNA or polypeptide. The SLC12A8 gene encodes the solute carrier family 12 member 8 protein. SLC12A8 is also known as CCC9. In some embodiments, SLC12A8 gene is a human SLC12A8. An exemplary SLC12A8 gene is represented by NCBI Gene ID No. 84561. An exemplary SLC12A8 mRNA sequence is represented by NCBI Ref. Seq. NM_024628. An exemplary amino acid sequence of an SLC12A8 polypeptide is represented by NCBI Ref. Seq. NP_78904.

As used herein “ADAM18” refers to a gene encoding an ADAM18 mRNA or polypeptide. The ADAM18 gene encodes ADAM metallopeptidase domain 18 protein. ADAM18 is also known as ADAM27 and tMDCIII. In some embodiments, ADAM18 gene is a human ADAM18. An exemplary ADAM18 gene is represented by NCBI Gene ID No. 8749. An exemplary ADAM18 mRNA sequence is represented by NCBI Ref. Seq. NM_14237. An exemplary amino acid sequence of an ADAM18 polypeptide is represented by NCBI Ref. Seq. NP_055052.

As used herein “BAG4” refers to a gene encoding a BAG4 mRNA or polypeptide. The BAG4 gene encodes BAG chaperone 4 protein. BAG4 is also known as SODD and BAG-4. In some embodiments, BAG4 gene is a human BAG4. An exemplary BAG4 gene is represented by NCBI Gene ID No. 9530. An exemplary BAG4 mRNA sequence is represented by NCBI Ref. Seq. NM_004874. An exemplary amino acid sequence of a BAG4 polypeptide is represented by NCBI Ref. Seq. NP_004865.

As used herein “TACC1” refers to a gene encoding a TACC1 mRNA or polypeptide. The TACC1 gene encodes transforming acidic coiled-coil containing protein 1 protein. TACC1 is also known as Ga55. In some embodiments, TACC1 gene is a human TACC1. An exemplary TACC1 gene is represented by NCBI Gene ID No. 6867. An exemplary TACC1 mRNA sequence is represented by NCBI Ref. Seq. NM_006283. An exemplary amino acid sequence of a TACC1 polypeptide is represented by NCBI Ref. Seq. NP_006274.

In some aspects, provided herein are FGFR2 fusion nucleic acid molecules comprising at least a portion of FGFR2 and at least a portion of another gene.

In some embodiments, an FGFR2 fusion nucleic acid molecule comprises at least a portion of FGFR2 and at least a portion of AARSD1, ARMS2, ATF7, BAIAP2L1, CCAR1, CCSER2, CGNL1, EBF1, FANK1, FOXP1, CAMK2G, FLJ40288, GUCY2D, IQGAP2, PAWR, FLNB, IKZF2, KHDRBS1, MYOZ1, PCDH15, PRKAR1A, PRRC2A, RABGAP1, SCIN, STAU1, STK4, TIFA, TLK1, TRIM54, APIP, ATE1, BICC1, TFEC, GRB2, KIAA1217, KIAA1598, MACF1, MYH9, NRAP, RBM20, SPICE1, TACC2, VTI1A, WAC, WARS, or ZMYM4. For example, in some embodiments, the FGFR2 fusion nucleic acid molecule is selected from FGFR2-AARSD1, FGFR2-ARMS2, FGFR2-ATF7, FGFR2-BAIAP2L1, FGFR2-CCAR1, FGFR2-CCSER2, FGFR2-CGNL1, FGFR2-EBF1, FGFR2-FANK1, CAMK2G-FGFR2, FLJ40288-FGFR2, GUCY2D-FGFR2, IQGAP2-FGFR2, PAWR-FGFR2, FGFR2-FLNB, FGFR2-FOXP1, FGFR2-IKZF2, FGFR2-KHDRBS1, FGFR2-MYOZ1, FGFR2-PCDH15, FGFR2-PRKAR1A, FGFR2-PRRC2A, FGFR2-RABGAP1, FGFR2-SCIN, FGFR2-STAU1, FGFR2-STK4, FGFR2-TIFA, FGFR2-TLK1, FGFR2-TRIM54, FGFR2-APIP, FGFR2-ATE1, FGFR2-BICC1, TFEC-FGFR2, FGFR2-GRB2, FGFR2-KIAA1217, FGFR2-KIAA1598, FGFR2-MACF1, FGFR2-MYH9, FGFR2-NRAP, FGFR2-RBM20, FGFR2-SPICE1, FGFR2-TACC2, FGFR2-VTI1A, FGFR2-WAC, FGFR2-WARS, or FGFR2-ZMYM4, wherein the order of the genes is in the 5′ to 3′ direction. Exemplary and non-limiting FGFR2 fusion nucleic acid molecules are described herein and/or in Tables 1-6, and/or in the Examples herein.

As used herein “AARSD1” refers to a gene encoding an AARSD1 mRNA or polypeptide. The AARSD1 gene encodes alanyl-tRNA synthetase domain containing 1 protein. AARSD1 is also known as MGC2744 and AlaXp. In some embodiments, AARSD1 gene is a human AARSD1. An exemplary AARSD1 gene is represented by NCBI Gene ID No. 80755. An exemplary AARSD1 mRNA sequence is represented by NCBI Ref. Seq. NM_001261434. An exemplary amino acid sequence of a AARSD1 polypeptide is represented by NCBI Ref. Seq. NP_001248363.

As used herein “ARMS2” refers to a gene encoding an ARMS2 mRNA or polypeptide. The ARMS2 gene encodes age-related maculopathy susceptibility 2 protein. ARMS2 is also known as ARMD8 and LOC387715. In some embodiments, ARMS2 gene is a human ARMS2. An exemplary ARMS2 gene is represented by NCBI Gene ID No. 387715. An exemplary ARMS2 mRNA sequence is represented by NCBI Ref. Seq. NM_001099667. An exemplary amino acid sequence of an ARMS2 polypeptide is represented by NCBI Ref. Seq. NP_001093137.

As used herein “ATF7” refers to a gene encoding an ATF7 mRNA or polypeptide. The ATF7 gene encodes activating transcription factor 7 protein. ATF7 is also known as ATFA. In some embodiments, ATF7 gene is a human ATF7. An exemplary ATF7 gene is represented by NCBI Gene ID No. 11016. An exemplary ATF7 mRNA sequence is represented by NCBI Ref. Seq. NM_006856. An exemplary amino acid sequence of an ATF7 polypeptide is represented by NCBI Ref. Seq. NP_006847.

As used herein “BAIAP2L1” refers to a gene encoding a BAIAP2L1 mRNA or polypeptide. The BAIAP2L1 gene encodes BAR/IMD domain containing adaptor protein 2 like 1 protein. BAIAP2L1 is also known as IRTKS. In some embodiments, BAIAP2L1 gene is a human BAIAP2L1. An exemplary BAIAP2L1 gene is represented by NCBI Gene ID No. 55971. An exemplary BAIAP2L1 mRNA sequence is represented by NCBI Ref. Seq. NM_018842. An exemplary amino acid sequence of a BAIAP2L1 polypeptide is represented by NCBI Ref. Seq. NP_061330.

As used herein “CCAR1” refers to a gene encoding a CCAR1 mRNA or polypeptide. The CCAR1 gene encodes cell division cycle and apoptosis regulator 1 protein. CCAR1 is also known as FLJ10590, CARP-1, and CARPI. In some embodiments, CCAR1 gene is a human CCAR1. An exemplary CCAR1 gene is represented by NCBI Gene ID No. 55749. An exemplary CCAR1 mRNA sequence is represented by NCBI Ref. Seq. NM_018237. An exemplary amino acid sequence of a CCAR1 polypeptide is represented by NCBI Ref. Seq. NP_060707.

As used herein “CCSER2” refers to a gene encoding a CCSER2 mRNA or polypeptide. The CCSER2 gene encodes coiled-coil serine rich protein 2 protein. CCSER2 is also known as Gcap14, FAM190B, KIAA1128, and bA486022.1. In some embodiments, CCSER2 gene is a human CCSER2. An exemplary CCSER2 gene is represented by NCBI Gene ID No. 54462. An exemplary CCSER2 mRNA sequence is represented by NCBI Ref. Seq. NM_018999. An exemplary amino acid sequence of a CCSER2 polypeptide is represented by NCBI Ref. Seq. NP_061872.

As used herein “CGNL1” refers to a gene encoding a CGNL1 mRNA or polypeptide. The CGNL1 gene encodes cingulin like 1 protein. CGNL1 is also known as JACOP, FLJ14957, KIAA1749, and PCING. In some embodiments, CGNL1 gene is a human CGNL1. An exemplary CGNL1 gene is represented by NCBI Gene ID No. 84952. An exemplary CGNL1 mRNA sequence is represented by NCBI Ref. Seq. NM_032866. An exemplary amino acid sequence of a CGNL1 polypeptide is represented by NCBI Ref. Seq. NP_116255.

As used herein “EBF1” refers to a gene encoding an EBF1 mRNA or polypeptide. The EBF1 gene encodes EBF transcription factor 1 protein. EBF1 is also known as EBF, COE1, OLF1, and O/E-1. In some embodiments, EBF1 gene is a human EBF1. An exemplary EBF1 gene is represented by NCBI Gene ID No. 1879. An exemplary EBF1 mRNA sequence is represented by NCBI Ref. Seq. NM_024007. An exemplary amino acid sequence of an EBF1 polypeptide is represented by NCBI Ref. Seq. NP_076870.

As used herein “FANK1” refers to a gene encoding a FANK1 mRNA or polypeptide. The FANK1 gene encodes fibronectin type III and ankyrin repeat domains 1 protein. FANK1 is also known as HSD13. In some embodiments, FANK1 gene is a human FANK1. An exemplary FANK1 gene is represented by NCBI Gene ID No. 92565. An exemplary FANK1 mRNA sequence is represented by NCBI Ref. Seq. NM_145235. An exemplary amino acid sequence of a FANK1 polypeptide is represented by NCBI Ref. Seq. NP_660278.

As used herein “FOXP1” refers to a gene encoding a FOXP1 mRNA or polypeptide. The FOXP1 gene encodes forkhead box P1 protein. FOXP1 is also known as MFH, QRF1, 12CC4, hFKH1B, and HSPC215. In some embodiments, FOXP1 gene is a human FOXP1. An exemplary FOXP1 gene is represented by NCBI Gene ID No. 27086. An exemplary FOXP1 mRNA sequence is represented by NCBI Ref. Seq. NM_032682. An exemplary amino acid sequence of a FOXP1 polypeptide is represented by NCBI Ref. Seq. NP_116071.

As used herein “CAMK2G” refers to a gene encoding a CAMK2G mRNA or polypeptide. The CAMK2G gene encodes calcium/calmodulin dependent protein kinase II gamma protein. CAMK2G is also known as CAMK, CAMKG, MRD59, and CAMK-II. In some embodiments, CAMK2G gene is a human CAMK2G. An exemplary CAMK2G gene is represented by NCBI Gene ID No. 818. An exemplary CAMK2G mRNA sequence is represented by NCBI Ref. Seq. NM_001222. An exemplary amino acid sequence of a CAMK2G polypeptide is represented by NCBI Ref. Seq. NP_001213.

As used herein “FLJ40288” refers to a gene encoding a FLJ40288 ncRNA. In some embodiments, an FLJ40288 gene is a human FLJ40288 gene. An exemplary FLJ40288 gene is represented by NCBI Gene ID No. 286023. An exemplary FLJ40288 ncRNA sequence is represented by NCBI Ref. Seq. NR_046323.

As used herein “GUCY2D” refers to a gene encoding a GUCY2D mRNA or polypeptide. The GUCY2D gene encodes guanylate cyclase 2D protein. GUCY2D is also known as LCA, CG-E, CYGD, LCA1, RCD2, CACD1, CORDS, CORD6, GUC2D, ROSGC, retGC, CSNB11, GUC1A4, RETGC-1, and ROS-GC1. In some embodiments, GUCY2D gene is a human GUCY2D. An exemplary GUCY2D gene is represented by NCBI Gene ID No. 3000. An exemplary GUCY2D mRNA sequence is represented by NCBI Ref. Seq. NM_000180. An exemplary amino acid sequence of a GUCY2D polypeptide is represented by NCBI Ref. Seq. NP_000171.

As used herein “IQGAP2” refers to a gene encoding an IQGAP2 mRNA or polypeptide. The IQGAP2 gene encodes IQ motif containing GTPase activating protein 2 protein. IQGAP2 is also known as LCA, CG-E, CYGD, LCA1, RCD2, CACD1, CORDS, CORD6, GUC2D, ROSGC, retGC, CSNB11, GUC1A4, RETGC-1, and ROS-GC1. In some embodiments, IQGAP2 gene is a human IQGAP2. An exemplary IQGAP2 gene is represented by NCBI Gene ID No. 10788. An exemplary IQGAP2 mRNA sequence is represented by NCBI Ref. Seq. NM_006633. An exemplary amino acid sequence of an IQGAP2 polypeptide is represented by NCBI Ref. Seq. NP_006624.

As used herein “PAWR” refers to a gene encoding a PAWR mRNA or polypeptide. The PAWR gene encodes pro-apoptotic WT1 regulator protein. PAWR is also known as PAR4 and Par-4. In some embodiments, PAWR gene is a human PAWR. An exemplary PAWR gene is represented by NCBI Gene ID No. 5074. An exemplary PAWR mRNA sequence is represented by NCBI Ref. Seq. NM_002583. An exemplary amino acid sequence of a PAWR polypeptide is represented by NCBI Ref. Seq. NP_002574.

As used herein “FLNB” refers to a gene encoding a FLNB mRNA or polypeptide. The FLNB gene encodes filamin B protein. FLNB is also known as AOI, FH1, SCT, TAP, LRS1, TABP, FLN-B, FLN1L, ABP-278, and ABP-280. In some embodiments, FLNB gene is a human FLNB. An exemplary FLNB gene is represented by NCBI Gene ID No. 2317. An exemplary FLNB mRNA sequence is represented by NCBI Ref. Seq. NM_001457. An exemplary amino acid sequence of a FLNB polypeptide is represented by NCBI Ref. Seq. NP_001448.

As used herein “IKZF2” refers to a gene encoding an IKZF2 mRNA or polypeptide. The IKZF2 gene encodes IKAROS family zinc finger 2 protein. IKZF2 is also known as ANF1A2, HELIOS, ZNF1A2, and ZNFN1A2. In some embodiments, IKZF2 gene is a human IKZF2. An exemplary IKZF2 gene is represented by NCBI Gene ID No. 22807. An exemplary IKZF2 mRNA sequence is represented by NCBI Ref. Seq. NM_001079526. An exemplary amino acid sequence of an IKZF2 polypeptide is represented by NCBI Ref. Seq. NP_001072994.

As used herein “KHDRBS1” refers to a gene encoding a KHDRBS1 mRNA or polypeptide. The KHDRBS1 gene encodes KH RNA binding domain containing, signal transduction associated 1 protein. KHDRBS1 is also known as p62, p68, and Sam68. In some embodiments, KHDRBS1 gene is a human KHDRBS1. An exemplary KHDRBS1 gene is represented by NCBI Gene ID No. 10657. An exemplary KHDRBS1 mRNA sequence is represented by NCBI Ref. Seq. NM_006559. An exemplary amino acid sequence of a KHDRBS1 polypeptide is represented by NCBI Ref. Seq. NP_006550.

As used herein “MYOZ1” refers to a gene encoding a MYOZ1 mRNA or polypeptide. The MYOZ1 gene encodes myozenin 1 protein. MYOZ1 is also known as p62, p68, and Sam68. In some embodiments, MYOZ1 gene is a human MYOZ1. An exemplary MYOZ1 gene is represented by NCBI Gene ID No. 58529. An exemplary MYOZ1 mRNA sequence is represented by NCBI Ref. Seq. NM_021245. An exemplary amino acid sequence of a MYOZ1 polypeptide is represented by NCBI Ref. Seq. NP_067068.

As used herein “PCDH15” refers to a gene encoding a PCDH15 mRNA or polypeptide. The PCDH15 gene encodes protocadherin related 15 protein. PCDH15 is also known as USH1F, CDHR15, and DFNB23. In some embodiments, PCDH15 gene is a human PCDH15. An exemplary PCDH15 gene is represented by NCBI Gene ID No. 65217. An exemplary PCDH15 mRNA sequence is represented by NCBI Ref. Seq. NM_033056. An exemplary amino acid sequence of a PCDH15 polypeptide is represented by NCBI Ref. Seq. NP_149045.

As used herein “PRKAR1A” refers to a gene encoding a PRKAR1A mRNA or polypeptide. The PRKAR1A gene encodes protein kinase cAMP-dependent type I regulatory subunit alpha protein. PRKAR1A is also known as CAR, CNC, CNC1, PKR1, TSE1, ADOHR, PPNAD1, PRKAR1, and ACRDYS1. In some embodiments, PRKAR1A gene is a human PRKAR1A. An exemplary PRKAR1A gene is represented by NCBI Gene ID No. 5573. An exemplary PRKAR1A mRNA sequence is represented by NCBI Ref. Seq. NM_001278433. An exemplary amino acid sequence of a PRKAR1A polypeptide is represented by NCBI Ref. Seq. NP_001265362.

As used herein “PRRC2A” refers to a gene encoding a PRRC2A mRNA or polypeptide. The PRRC2A gene encodes proline rich coiled-coil 2A protein. PRRC2A is also known as CAR, CNC, CNC1, PKR1, TSE1, ADOHR, PPNAD1, PRKAR1, and ACRDYS1. In some embodiments, PRRC2A gene is a human PRRC2A. An exemplary PRRC2A gene is represented by NCBI Gene ID No. 7916. An exemplary PRRC2A mRNA sequence is represented by NCBI Ref. Seq. NM_004638. An exemplary amino acid sequence of a PRRC2A polypeptide is represented by NCBI Ref. Seq. NP_004629.

As used herein “RABGAP1” refers to a gene encoding a RABGAP1 mRNA or polypeptide. The RABGAP1 gene encodes RAB GTPase activating protein 1 protein. RABGAP1 is also known as GAPCENA and TBC1D11. In some embodiments, RABGAP1 gene is a human RABGAP1. An exemplary RABGAP1 gene is represented by NCBI Gene ID No. 23637. An exemplary RABGAP1 mRNA sequence is represented by NCBI Ref. Seq. NM_012197. An exemplary amino acid sequence of a RABGAP1 polypeptide is represented by NCBI Ref. Seq. NP_036329.

As used herein “SCIN” refers to a gene encoding a SCIN mRNA or polypeptide. The SCIN gene encodes scinderin protein. SCIN is also known as KIAA1905. In some embodiments, SCIN gene is a human SCIN. An exemplary SCIN gene is represented by NCBI Gene ID No. 85477. An exemplary SCIN mRNA sequence is represented by NCBI Ref. Seq. NM_033128. An exemplary amino acid sequence of a SCIN polypeptide is represented by NCBI Ref. Seq. NP_149119.

As used herein “STAU1” refers to a gene encoding a STAU1 mRNA or polypeptide. The STAU1 gene encodes staufen double-stranded RNA binding protein 1 protein. STAU1 is also known as STAU and PPP1R150. In some embodiments, STAU1 gene is a human STAU1. An exemplary STAU1 gene is represented by NCBI Gene ID No. 6780. An exemplary STAU1 mRNA sequence is represented by NCBI Ref. Seq. NM_004602. An exemplary amino acid sequence of a STAU1 polypeptide is represented by NCBI Ref. Seq. NP_004593.

As used herein “STK4” refers to a gene encoding a STK4 mRNA or polypeptide. The STK4 gene encodes serine/threonine kinase 4 protein. STK4 is also known as KRS2, MST1, and YSK3. In some embodiments, STK4 gene is a human STK4. An exemplary STK4 gene is represented by NCBI Gene ID No. 6789. An exemplary STK4 mRNA sequence is represented by NCBI Ref. Seq. NM_006282. An exemplary amino acid sequence of a STK4 polypeptide is represented by NCBI Ref. Seq. NP_006273.

As used herein “TIFA” refers to a gene encoding a TIFA mRNA or polypeptide. The TIFA gene encodes the TRAF interacting protein with forkhead associated domain protein. TIFA is also known as T2BP, T6BP, and TIFAA. In some embodiments, TIFA gene is a human TIFA. An exemplary TIFA gene is represented by NCBI Gene ID No. 92610. An exemplary TIFA mRNA sequence is represented by NCBI Ref. Seq. NM_052864. An exemplary amino acid sequence of a TIFA polypeptide is represented by NCBI Ref. Seq. NP_443096.

As used herein “TLK1” refers to a gene encoding a TLK1 mRNA or polypeptide. The TLK1 gene encodes the tousled like kinase 1 protein. TLK1 is also known as PKU-beta. In some embodiments, TLK1 gene is a human TLK1. An exemplary TLK1 gene is represented by NCBI Gene ID No. 9874. An exemplary TLK1 mRNA sequence is represented by NCBI Ref. Seq. NM_012290. An exemplary amino acid sequence of a TLK1 polypeptide is represented by NCBI Ref. Seq. NP_036422.

As used herein “TRIM54” refers to a gene encoding a TRIM54 mRNA or polypeptide. The TRIM54 gene encodes the tripartite motif containing 54 protein. TRIM54 is also known as MURF, MURF-3, RNF30, and muRF3. In some embodiments, TRIM54 gene is a human TRIM54. An exemplary TRIM54 gene is represented by NCBI Gene ID No. 57159. An exemplary TRIM54 mRNA sequence is represented by NCBI Ref. Seq. NM_032546. An exemplary amino acid sequence of a TRIM54 polypeptide is represented by NCBI Ref. Seq. NP_115935.

As used herein “APIP” refers to a gene encoding an APIP mRNA or polypeptide. The APIP gene encodes the APAF1 interacting protein protein. APIP is also known as APIP2, CGI-29, CGI29, MMRP19, and hAPIP. In some embodiments, APIP gene is a human APIP. An exemplary APIP gene is represented by NCBI Gene ID No. 51074. An exemplary APIP mRNA sequence is represented by NCBI Ref. Seq. NM_015957. An exemplary amino acid sequence of an APIP polypeptide is represented by NCBI Ref. Seq. NP_057041.

As used herein “ATE1” refers to a gene encoding an ATE1 mRNA or polypeptide. The ATE1 gene encodes the arginyltransferase 1 protein. ATE1 is also known as APIP2, CGI-29, CGI29, MMRP19, and hAPIP. In some embodiments, ATE1 gene is a human ATE1. An exemplary ATE1 gene is represented by NCBI Gene ID No. 11101. An exemplary ATE1 mRNA sequence is represented by NCBI Ref. Seq. NM_007041. An exemplary amino acid sequence of an ATE1 polypeptide is represented by NCBI Ref. Seq. NP_008972.

As used herein “BICC1” refers to a gene encoding a BICC1 mRNA or polypeptide. The BICC1 gene encodes the BicC family RNA binding protein 1 protein. BICC1 is also known as BICC and CYSRD. In some embodiments, BICC1 gene is a human BICC1. An exemplary BICC1 gene is represented by NCBI Gene ID No. 80114. An exemplary BICC1 mRNA sequence is represented by NCBI Ref. Seq. NM_001080512. An exemplary amino acid sequence of a BICC1 polypeptide is represented by NCBI Ref. Seq. NP_001073981.

As used herein “TFEC” refers to a gene encoding a TFEC mRNA or polypeptide. The TFEC gene encodes the transcription factor EC protein. TFEC is also known as TCFEC, TFE-C, TFEC-L, TFECL, bHLHe34, and hTFEC-L. In some embodiments, TFEC gene is a human TFEC. An exemplary TFEC gene is represented by NCBI Gene ID No. 22797. An exemplary TFEC mRNA sequence is represented by NCBI Ref. Seq. NM_012252. An exemplary amino acid sequence of a TFEC polypeptide is represented by NCBI Ref. Seq. NP_036384.

As used herein “GRB2” refers to a gene encoding a GRB2 mRNA or polypeptide. The GRB2 gene encodes the growth factor receptor bound protein 2 protein. GRB2 is also known as ASH, EGFRBP-GRB2, Grb3-3, MST084, MSTP084, and NCKAP2. In some embodiments, GRB2 gene is a human GRB2. An exemplary GRB2 gene is represented by NCBI Gene ID No. 2885. An exemplary GRB2 mRNA sequence is represented by NCBI Ref. Seq. NM_002086. An exemplary amino acid sequence of a GRB2 polypeptide is represented by NCBI Ref. Seq. NP_002077.

As used herein “KIAA1217” refers to a gene encoding a KIAA1217 mRNA or polypeptide. The KIAA1217 gene encodes the KIAA1217 protein. KIAA1217 is also known as ETL4 and SKT. In some embodiments, KIAA1217 gene is a human KIAA1217. An exemplary KIAA1217 gene is represented by NCBI Gene ID No. 56243. An exemplary KIAA1217 mRNA sequence is represented by NCBI Ref. Seq. NM_019590. An exemplary amino acid sequence of a KIAA1217 polypeptide is represented by NCBI Ref. Seq. NP_062536.

As used herein “KIAA1598” refers to a gene encoding a KIAA1598 mRNA or polypeptide. The KIAA1598 gene encodes the KIAA1598 protein. KIAA1598 is also known as shootin-1 and SHTN1. In some embodiments, KIAA1598 gene is a human KIAA1598. An exemplary KIAA1598 gene is represented by NCBI Gene ID No. 57698. An exemplary KIAA1598 mRNA sequence is represented by NCBI Ref. Seq. NM_018330. An exemplary amino acid sequence of a KIAA1598 polypeptide is represented by NCBI Ref. Seq. NP_060800.

As used herein “MACF1” refers to a gene encoding a MACF1 mRNA or polypeptide. The MACF1 gene encodes the microtubule actin crosslinking factor 1 protein. MACF1 is also known as ABP620, ACF7, LIS9, Lnc-PMIF, MACF, and OFC4. In some embodiments, MACF1 gene is a human MACF1. An exemplary MACF1 gene is represented by NCBI Gene ID No. 23499. An exemplary MACF1 mRNA sequence is represented by NCBI Ref. Seq. NM_012090. An exemplary amino acid sequence of a MACF1 polypeptide is represented by NCBI Ref. Seq. NP_036222.

As used herein “MYH9” refers to a gene encoding a MYH9 mRNA or polypeptide. The MYH9 gene encodes the myosin heavy chain 9 protein. MYH9 is also known as BDPLT6, DFNA17, EPSTS, FTNS, MATINS, MHA, NMHC-II-A, NMMHC-IIA, and NMMHCA. In some embodiments, MYH9 gene is a human MYH9. An exemplary MYH9 gene is represented by NCBI Gene ID No. 4627. An exemplary MYH9 mRNA sequence is represented by NCBI Ref. Seq. NM_002473. An exemplary amino acid sequence of a MYH9 polypeptide is represented by NCBI Ref. Seq. NP_002464.

As used herein “NRAP” refers to a gene encoding a NRAP mRNA or polypeptide. The NRAP gene encodes the nebulin related anchoring protein protein. NRAP is also known as N-RAP. In some embodiments, NRAP gene is a human NRAP. An exemplary NRAP gene is represented by NCBI Gene ID No. 4892. An exemplary NRAP mRNA sequence is represented by NCBI Ref. Seq. NM_006175. An exemplary amino acid sequence of a NRAP polypeptide is represented by NCBI Ref. Seq. NP_006166.

As used herein “RBM20” refers to a gene encoding a RBM20 mRNA or polypeptide. The RBM20 gene encodes the RNA binding motif protein 20 protein. In some embodiments, RBM20 gene is a human RBM20. An exemplary RBM20 gene is represented by NCBI Gene ID No. 282996. An exemplary RBM20 mRNA sequence is represented by NCBI Ref. Seq. NM_001134363. An exemplary amino acid sequence of a RBM20 polypeptide is represented by NCBI Ref. Seq. NP_001127835.

As used herein “SPICE1” refers to a gene encoding a SPICE1 mRNA or polypeptide. The SPICE1 gene encodes the spindle and centriole associated protein 1 protein. SPICE1 is also known as CCDC52 and SPICE. In some embodiments, SPICE1 gene is a human SPICE1. An exemplary SPICE1 gene is represented by NCBI Gene ID No. 152185. An exemplary SPICE1 mRNA sequence is represented by NCBI Ref. Seq. NM_144718. An exemplary amino acid sequence of a SPICE1 polypeptide is represented by NCBI Ref. Seq. NP_653319.

As used herein “TACC2” refers to a gene encoding a TACC2 mRNA or polypeptide. The TACC2 gene encodes the transforming acidic coiled-coil containing protein 2 protein. TACC2 is also known as AZU-1 and ECTACC. In some embodiments, TACC2 gene is a human TACC2. An exemplary TACC2 gene is represented by NCBI Gene ID No. 10579. An exemplary TACC2 mRNA sequence is represented by NCBI Ref. Seq. NM_006997. An exemplary amino acid sequence of a TACC2 polypeptide is represented by NCBI Ref. Seq. NP_008928.

As used herein “VTI1A” refers to a gene encoding a VTI1A mRNA or polypeptide. The VTI1A gene encodes the vesicle transport through interaction with t-SNAREs 1A protein. VTI1A is also known as MMDS3, MVti1, VTI1RP2, and Vtil-rp2. In some embodiments, VTI1A gene is a human VTI1A. An exemplary VTI1A gene is represented by NCBI Gene ID No. 143187. An exemplary VTI1A mRNA sequence is represented by NCBI Ref. Seq. NM_145206. An exemplary amino acid sequence of a VTI1A polypeptide is represented by NCBI Ref. Seq. NP_660207.

As used herein “WAC” refers to a gene encoding a WAC mRNA or polypeptide. The WAC gene encodes the WW domain containing adaptor with coiled-coil protein. WAC is also known as BM-016, DESSH, PRO1741, and Wwp4. In some embodiments, WAC gene is a human WAC. An exemplary WAC gene is represented by NCBI Gene ID No. 51322. An exemplary WAC mRNA sequence is represented by NCBI Ref. Seq. NM_016628. An exemplary amino acid sequence of a WAC polypeptide is represented by NCBI Ref. Seq. NP_057712.

As used herein “WARS” refers to a gene encoding a WARS mRNA or polypeptide. The WARS gene encodes the tryptophanyl-tRNA synthetase protein. WARS is also known as TrpRS, WRS, and Wars1. In some embodiments, WARS gene is a human WARS. An exemplary WARS gene is represented by NCBI Gene ID No. 7453. An exemplary WARS mRNA sequence is represented by NCBI Ref. Seq. NM_004184. An exemplary amino acid sequence of a WARS polypeptide is represented by NCBI Ref. Seq. NP_004175.

As used herein “ZMYM4” refers to a gene encoding a ZMYM4 mRNA or polypeptide. The ZMYM4 gene encodes the zinc finger MYM-type containing 4 protein. ZMYM4 is also known as CDIR, MYM, ZNF198L3, and ZNF262. In some embodiments, ZMYM4 gene is a human ZMYM4. An exemplary ZMYM4 gene is represented by NCBI Gene ID No. 9202. An exemplary ZMYM4 mRNA sequence is represented by NCBI Ref. Seq. NM_005095. An exemplary amino acid sequence of a ZMYM4 polypeptide is represented by NCBI Ref. Seq. NP_005086.

In some aspects, provided herein are FGFR3 fusion nucleic acid molecules comprising at least a portion of FGFR3 and at least a portion of another gene.

In some embodiments, an FGFR3 fusion nucleic acid molecule comprises at least a portion of FGFR3 and at least a portion of CCT5, CNOT4, TNIP2, IGH, TACC3, ADD1, or WHSC1. For example, in some embodiments, the FGFR3 fusion nucleic acid molecule is selected from FGFR3-CCT5, FGFR3-CNOT4, FGFR3-TNIP2, FGFR3-ADD1, FGFR3-IGH, FGFR3-TACC3, or FGFR3-WHSC1, wherein the order of the genes is in the 5′ to 3′ direction. Exemplary and non-limiting FGFR3 fusion nucleic acid molecules are described herein and/or in Tables 1-6, and/or in the Examples herein.

As used herein “CCT5” refers to a gene encoding a CCT5 mRNA or polypeptide. The CCT5 gene encodes the chaperonin containing TCP1 subunit 5 protein. CCT5 is also known as CCT-epsilon, CCTE, HEL-S-69, PNAS-102, and TCP-1-epsilon. In some embodiments, CCT5 gene is a human CCT5. An exemplary CCT5 gene is represented by NCBI Gene ID No. 22948. An exemplary CCT5 mRNA sequence is represented by NCBI Ref. Seq. NM_012073. An exemplary amino acid sequence of a CCT5 polypeptide is represented by NCBI Ref. Seq. NP_036205.

As used herein “CNOT4” refers to a gene encoding a CNOT4 mRNA or polypeptide. The CNOT4 gene encodes the CCR4-NOT transcription complex subunit 4 protein. CNOT4 is also known as CLONE243, NOT4, and NOT4H. In some embodiments, CNOT4 gene is a human CNOT4. An exemplary CNOT4 gene is represented by NCBI Gene ID No. 4850. An exemplary CNOT4 mRNA sequence is represented by NCBI Ref. Seq. NM_013316. An exemplary amino acid sequence of a CNOT4 polypeptide is represented by NCBI Ref. Seq. NP_037448.

As used herein “TNIP2” refers to a gene encoding a TNIP2 mRNA or polypeptide. The TNIP2 gene encodes the TNFAIP3 interacting protein 2 protein. TNIP2 is also known as ABIN2, FLIP1, and KLIP. In some embodiments, TNIP2 gene is a human TNIP2. An exemplary TNIP2 gene is represented by NCBI Gene ID No. 79155. An exemplary TNIP2 mRNA sequence is represented by NCBI Ref. Seq. NM_024309. An exemplary amino acid sequence of a TNIP2 polypeptide is represented by NCBI Ref. Seq. NP_077285.

As used herein “IGH” refers to a gene encoding an IGH mRNA or polypeptide. The IGH gene encodes the immunoglobulin heavy locus protein. IGH is also known as IGD1, IGH.1 @, IGH@, IGHD@, IGHDY1, IGHJ, IGHJ@, IGHV, and IGHV@. In some embodiments, IGH gene is a human IGH. An exemplary IGH gene is represented by NCBI Gene ID No. 3492. An exemplary IGH DNA sequence is represented by NCBI Ref. Seq. NG_001019.

As used herein “TACC3” refers to a gene encoding a TACC3 mRNA or polypeptide. The TACC3 gene encodes the transforming acidic coiled-coil containing protein 3 protein. TACC3 is also known as ERIC-1, ERIC1, Tacc4, and maskin. In some embodiments, TACC3 gene is a human TACC3. An exemplary TACC3 gene is represented by NCBI Gene ID No. 10460. An exemplary TACC3 mRNA sequence is represented by NCBI Ref. Seq. NM_006342. An exemplary amino acid sequence of a TACC3 polypeptide is represented by NCBI Ref. Seq. NP_006333.

As used herein “ADD1” refers to a gene encoding an ADD1 mRNA or polypeptide. The ADD1 gene encodes the adducing 1 protein. ADD1 is also known as ADDA. In some embodiments, ADD1 gene is a human ADD1. An exemplary ADD1 gene is represented by NCBI Gene ID No. 118. An exemplary ADD1 mRNA sequence is represented by NCBI Ref. Seq. NM_001119. An exemplary amino acid sequence of an ADD1 polypeptide is represented by NCBI Ref. Seq. NP_001110.

As used herein “WHSC1” refers to a gene encoding a WHSC1 mRNA or polypeptide. The WHSC1 gene encodes the Wolf-Hirschhorn syndrome candidate 1 protein. WHSC1 is also known as KMT3F, KMT3G, MMSET, REIIBP, TRX5, WHS, and NSD2. In some embodiments, WHSC1 gene is a human WHSC1. An exemplary WHSC1 gene is represented by NCBI Gene ID No. 7468. An exemplary WHSC1 mRNA sequence is represented by NCBI Ref. Seq. NM_133330. An exemplary amino acid sequence of a WHSC1 polypeptide is represented by NCBI Ref. Seq. NP_579877.

In some aspects, provided herein are MET fusion nucleic acid molecules comprising at least a portion of MET and at least a portion of another gene.

In some embodiments, a MET fusion nucleic acid molecule comprises at least a portion of MET and at least a portion of LDHA, CNTNAP2, HBP1, SNRNP70, CAPZA2, or ST7. For example, in some embodiments, the MET fusion nucleic acid molecule is selected from MET-LDHA, CNTNAP2-MET, HBP1-MET, SNRNP70-MET, MET-CAPZA2, or ST7-MET, wherein the order of the genes is in the 5′ to 3′ direction. Exemplary and non-limiting MET fusion nucleic acid molecules are described herein and/or in Tables 1-6, and/or in the Examples herein.

As used herein “LDHA” refers to a gene encoding a LDHA mRNA or polypeptide. The LDHA gene encodes the lactate dehydrogenase A protein. LDHA is also known as GSD11, HEL-S-133P, LDHM, and PIG19. In some embodiments, LDHA gene is a human LDHA. An exemplary LDHA gene is represented by NCBI Gene ID No. 3939. An exemplary LDHA mRNA sequence is represented by NCBI Ref. Seq. NM_005566. An exemplary amino acid sequence of a LDHA polypeptide is represented by NCBI Ref. Seq. NP_005557.

As used herein “CNTNAP2” refers to a gene encoding a CNTNAP2 mRNA or polypeptide. The CNTNAP2 gene encodes the contacting associated protein 2 protein. CNTNAP2 is also known as AUTS15, CASPR2, CDFE, NRXN4, and PTHSL1. In some embodiments, CNTNAP2 gene is a human CNTNAP2. An exemplary CNTNAP2 gene is represented by NCBI Gene ID No. 26047. An exemplary CNTNAP2 mRNA sequence is represented by NCBI Ref. Seq. NM_014141. An exemplary amino acid sequence of a CNTNAP2 polypeptide is represented by NCBI Ref. Seq. NP_054860.

As used herein “HBP1” refers to a gene encoding a HBP1 mRNA or polypeptide. The HBP1 gene encodes the HMG-box transcription factor 1 protein. In some embodiments, HBP1 gene is a human HBP1. An exemplary HBP1 gene is represented by NCBI Gene ID No. 26959. An exemplary HBP1 mRNA sequence is represented by NCBI Ref. Seq. NM_012257. An exemplary amino acid sequence of a HBP1 polypeptide is represented by NCBI Ref. Seq. NP_036389.

As used herein “SNRNP70” refers to a gene encoding a SNRNP70 mRNA or polypeptide. The SNRNP70 gene encodes the small nuclear ribonucleoprotein U1 subunit 70 protein. SNRNP70 is also known as RNPU1Z, RPU1, SNRP70, Snp1, U1-70K, U170K, U1AP, and U1RNP. In some embodiments, SNRNP70 gene is a human SNRNP70. An exemplary SNRNP70 gene is represented by NCBI Gene ID No. 6625. An exemplary SNRNP70 mRNA sequence is represented by NCBI Ref. Seq. NM_003089. An exemplary amino acid sequence of a SNRNP70 polypeptide is represented by NCBI Ref. Seq. NP_003080.

As used herein “CAPZA2” refers to a gene encoding a CAPZA2 mRNA or polypeptide. The CAPZA2 gene encodes the capping actin protein of muscle Z-line subunit alpha 2 protein. CAPZA2 is also known as CAPPA2 and CAPZ. In some embodiments, CAPZA2 gene is a human CAPZA2. An exemplary CAPZA2 gene is represented by NCBI Gene ID No. 830. An exemplary CAPZA2 mRNA sequence is represented by NCBI Ref. Seq. NM_006136. An exemplary amino acid sequence of a CAPZA2 polypeptide is represented by NCBI Ref. Seq. NP_006127.

As used herein “ST7” refers to a gene encoding a ST7 mRNA or polypeptide. The ST7 gene encodes the suppression of tumorigenicity 7 protein. ST7 is also known as ETS7q, FAM4A, FAM4A1, HELG, RAY1, SEN4, and TSG7. In some embodiments, ST7 gene is a human ST7. An exemplary ST7 gene is represented by NCBI Gene ID No. 7982. An exemplary ST7 mRNA sequence is represented by NCBI Ref. Seq. NM_018412. An exemplary amino acid sequence of a ST7 polypeptide is represented by NCBI Ref. Seq. NP_060882.

In some aspects, provided herein are NTRK1 fusion nucleic acid molecules comprising at least a portion of NTRK1 and at least a portion of another gene.

In some embodiments, an NTRK1 fusion nucleic acid molecule comprises at least a portion of NTRK1 and at least a portion of MEF2D. For example, in some embodiments, the NTRK1 fusion nucleic acid molecule is an NTRK1-MEF2D fusion nucleic acid molecule, wherein the order of the genes is in the 5′ to 3′ direction. Exemplary and non-limiting NTRK1 fusion nucleic acid molecules are described herein and/or in Tables 2 and 6, and/or in the Examples herein.

As used herein “MEF2D” refers to a gene encoding an MEF2D mRNA or polypeptide. The MEF2D gene encodes the myocyte enhancer factor 2D protein. In some embodiments, MEF2D gene is a human MEF2D. An exemplary MEF2D gene is represented by NCBI Gene ID No. 4209. An exemplary MEF2D mRNA sequence is represented by NCBI Ref. Seq. NM_005920. An exemplary amino acid sequence of an MEF2D polypeptide is represented by NCBI Ref. Seq. NP_005911.

In some aspects, provided herein are RAF1 fusion nucleic acid molecules comprising at least a portion of RAF1 and at least a portion of another gene.

In some embodiments, a RAF1 fusion nucleic acid molecule comprises at least a portion of RAF1 and at least a portion of POC1A, SYN2, TRAK1, or ZFYVE20. For example, in some embodiments, the RAF1 fusion nucleic acid molecule is selected from POC1A-RAF1, SYN2-RAF1, ZFYVE20-RAF1, or RAF1-TRAK1, wherein the order of the genes is in the 5′ to 3′ direction. Exemplary and non-limiting RAF1 fusion nucleic acid molecules are described herein and/or in Tables 1-6, and/or in the Examples herein.

As used herein “POC1A” refers to a gene encoding a POC1A mRNA or polypeptide. The POC1A gene encodes the POC1 centriolar protein A protein. POC1A is also known as PIX2, SOFT, and WDR51A. In some embodiments, POC1A gene is a human POC1A. An exemplary POC1A gene is represented by NCBI Gene ID No. 25886. An exemplary POC1A mRNA sequence is represented by NCBI Ref. Seq. NM_015426. An exemplary amino acid sequence of a POC1A polypeptide is represented by NCBI Ref. Seq. NP_056241.

As used herein “SYN2” refers to a gene encoding a SYN2 mRNA or polypeptide. The SYN2 gene encodes the synapsin II protein. SYN2 is also known as SYNII. In some embodiments, SYN2 gene is a human SYN2. An exemplary SYN2 gene is represented by NCBI Gene ID No. 6854. An exemplary SYN2 mRNA sequence is represented by NCBI Ref. Seq. NM_003178. An exemplary amino acid sequence of a SYN2 polypeptide is represented by NCBI Ref. Seq. NP_003169.

As used herein “TRAK1” refers to a gene encoding a TRAK1 mRNA or polypeptide. The TRAK1 gene encodes the trafficking kinesin protein 1 protein. TRAK1 is also known as DEE68, EIEE68, MILT1, and OIP106. In some embodiments, TRAK1 gene is a human TRAK1. An exemplary TRAK1 gene is represented by NCBI Gene ID No. 22906. An exemplary TRAK1 mRNA sequence is represented by NCBI Ref. Seq. NM_014965. An exemplary amino acid sequence of a TRAK1 polypeptide is represented by NCBI Ref. Seq. NP_055780.

As used herein “ZFYVE20” refers to a gene encoding a ZFYVE20 mRNA or polypeptide. The ZFYVE20 gene encodes the Rabenosyn-5 protein. ZFYVE20 is also known as Rabenosyn-5 and RBSN. In some embodiments, ZFYVE20 gene is a human ZFYVE20. An exemplary ZFYVE20 gene is represented by NCBI Gene ID No. 64145. An exemplary ZFYVE20 mRNA sequence is represented by NCBI Ref. Seq. NM_022340. An exemplary amino acid sequence of a ZFYVE20 polypeptide is represented by NCBI Ref. Seq. NP_071735.

In some aspects, provided herein are RET fusion nucleic acid molecules comprising at least a portion of RET and at least a portion of another gene.

In some embodiments, a RET fusion nucleic acid molecule comprises at least a portion of RET and at least a portion of ADCY1, NPY4R, PAWR, ALOX5, ARID5B, DHX32, PDE5A, ZNF365, BAIAP2L1, CSGALNACT2, GPHN, NCOA4, RASGEF1A, KIAA1217, CCDC6, ERC1, KIF5B, TRIM24, or VCL. For example, in some embodiments, the RET fusion nucleic acid molecule is selected from RET-ADCY1, RET-NPY4R, RET-PAWR, ALOX5-RET, ARID5B-RET, DHX32-RET, PDE5A-RET, ZNF365-RET, BAIAP2L1-RET, RET-CSGALNACT2, RET-GPHN, NCOA4-RET, RET-RASGEF1A, KIAA1217-RET, CCDC6-RET, ERC1-RET, KIF5B-RET, TRIM24-RET, or VCL-RET, wherein the order of the genes is in the 5′ to 3′ direction. Exemplary and non-limiting RET fusion nucleic acid molecules are described herein and/or in Tables 1-6, and/or in the Examples herein.

As used herein “ADCY1” refers to a gene encoding an ADCY1 mRNA or polypeptide. The ADCY1 gene encodes the adenylate cyclase 1 protein. ADCY1 is also known as AC1 and DFNB44. In some embodiments, ADCY1 gene is a human ADCY1. An exemplary ADCY1 gene is represented by NCBI Gene ID No. 107. An exemplary ADCY1 mRNA sequence is represented by NCBI Ref. Seq. NM_021116. An exemplary amino acid sequence of an ADCY1 polypeptide is represented by NCBI Ref. Seq. NP_066939.

As used herein “NPY4R” refers to a gene encoding a NPY4R mRNA or polypeptide. The NPY4R gene encodes the neuropeptide Y receptor Y4 protein. NPY4R is also known as NPY4-R, PP1, PPYR1, and Y4. In some embodiments, NPY4R gene is a human NPY4R. An exemplary NPY4R gene is represented by NCBI Gene ID No. 5540. An exemplary NPY4R mRNA sequence is represented by NCBI Ref. Seq. NM_005972. An exemplary amino acid sequence of a NPY4R polypeptide is represented by NCBI Ref. Seq. NP_005963.

As used herein “PAWR” refers to a gene encoding a PAWR mRNA or polypeptide. The PAWR gene encodes pro-apoptotic WT1 regulator protein. PAWR is also known as PAR4 and Par-4. In some embodiments, PAWR gene is a human PAWR. An exemplary PAWR gene is represented by NCBI Gene ID No. 5074. An exemplary PAWR mRNA sequence is represented by NCBI Ref. Seq. NM_002583. An exemplary amino acid sequence of a PAWR polypeptide is represented by NCBI Ref. Seq. NP_002574.

As used herein “ALOX5” refers to a gene encoding an ALOX5 mRNA or polypeptide. The ALOX5 gene encodes arachidonate 5-lipoxygenase protein. ALOX5 is also known as 5-LO, 5-LOX, 5LPG, and LOG5. In some embodiments, ALOX5 gene is a human ALOX5. An exemplary ALOX5 gene is represented by NCBI Gene ID No. 240. An exemplary ALOX5 mRNA sequence is represented by NCBI Ref. Seq. NM_000698. An exemplary amino acid sequence of an ALOX5 polypeptide is represented by NCBI Ref. Seq. NP_000689.

As used herein “ARID5B” refers to a gene encoding an ARID5B mRNA or polypeptide. The ARID5B gene encodes AT-rich interaction domain 5B protein. ARID5B is also known as 5 DESRT, MRF-2, and MRF2. In some embodiments, ARID5B gene is a human ARID5B. An exemplary ARID5B gene is represented by NCBI Gene ID No. 84159. An exemplary ARID5B mRNA sequence is represented by NCBI Ref. Seq. NM_032199. An exemplary amino acid sequence of an ARID5B polypeptide is represented by NCBI Ref. Seq. NP_115575.

As used herein “DHX32” refers to a gene encoding a DHX32 mRNA or polypeptide. The DHX32 gene encodes DEAH-box helicase 32 protein. DHX32 is also known as DDX32 and DHLP1. In some embodiments, DHX32 gene is a human DHX32. An exemplary DHX32 gene is represented by NCBI Gene ID No. 55760. An exemplary DHX32 mRNA sequence is represented by NCBI Ref. Seq. NM_018180. An exemplary amino acid sequence of a DHX32 polypeptide is represented by NCBI Ref. Seq. NP_060650.

As used herein “PDE5A” refers to a gene encoding a PDE5A mRNA or polypeptide. The PDE5A gene encodes phosphodiesterase 5A protein. PDE5A is also known as CGB-PDE, CN5A, and PDE5. In some embodiments, PDE5A gene is a human PDE5A. An exemplary PDE5A gene is represented by NCBI Gene ID No. 8654. An exemplary PDE5A mRNA sequence is represented by NCBI Ref. Seq. NM_001083. An exemplary amino acid sequence of a PDE5A polypeptide is represented by NCBI Ref. Seq. NP_001074.

As used herein “ZNF365” refers to a gene encoding a ZNF365 mRNA or polypeptide. The ZNF365 gene encodes zinc finger protein 365 protein. ZNF365 is also known as Su48, UAN, and ZNF365D. In some embodiments, ZNF365 gene is a human ZNF365. An exemplary ZNF365 gene is represented by NCBI Gene ID No. 22891. An exemplary ZNF365 mRNA sequence is represented by NCBI Ref. Seq. NM_014951. An exemplary amino acid sequence of a ZNF365 polypeptide is represented by NCBI Ref. Seq. NP_055766.

As used herein “BAIAP2L1” refers to a gene encoding a BAIAP2L1 mRNA or polypeptide. The BAIAP2L1 gene encodes BAR/IMD domain containing adaptor protein 2 like 1 protein. BAIAP2L1 is also known as IRTKS. In some embodiments, BAIAP2L1 gene is a human BAIAP2L1. An exemplary BAIAP2L1 gene is represented by NCBI Gene ID No. 55971. An exemplary BAIAP2L1 mRNA sequence is represented by NCBI Ref. Seq. NM_018842. An exemplary amino acid sequence of a BAIAP2L1 polypeptide is represented by NCBI Ref. Seq. NP_061330.

As used herein “CSGALNACT2” refers to a gene encoding a CSGALNACT2 mRNA or polypeptide. The CSGALNACT2 gene encodes chondroitin sulfate N-acetylgalactosaminyltransferase 2 protein. CSGALNACT2 is also known as CHGN2, ChGn-2, GALNACT-2, GALNACT2, PRO0082, and beta4GalNAcT. In some embodiments, CSGALNACT2 gene is a human CSGALNACT2. An exemplary CSGALNACT2 gene is represented by NCBI Gene ID No. 55454. An exemplary CSGALNACT2 mRNA sequence is represented by NCBI Ref. Seq. NM_018590. An exemplary amino acid sequence of a CSGALNACT2 polypeptide is represented by NCBI Ref. Seq. NP_061060.

As used herein “GPHN” refers to a gene encoding a GPHN mRNA or polypeptide. The GPHN gene encodes gephyrin protein. GPHN is also known as GEPH, GPH, GPHRYN, HKPX1, and MOCODC. In some embodiments, GPHN gene is a human GPHN. An exemplary GPHN gene is represented by NCBI Gene ID No. 10243. An exemplary GPHN mRNA sequence is represented by NCBI Ref. Seq. NM_020806. An exemplary amino acid sequence of a GPHN polypeptide is represented by NCBI Ref. Seq. NP_065857.

As used herein “NCOA4” refers to a gene encoding a NCOA4 mRNA or polypeptide. The NCOA4 gene encodes nuclear receptor coactivator 4 protein. NCOA4 is also known as ARA70, ELE1, PTC3, and RFG. In some embodiments, NCOA4 gene is a human NCOA4. An exemplary NCOA4 gene is represented by NCBI Gene ID No. 8031. An exemplary NCOA4 mRNA sequence is represented by NCBI Ref. Seq. NM_005437. An exemplary amino acid sequence of a NCOA4 polypeptide is represented by NCBI Ref. Seq. NP_005428.

As used herein “RASGEF1A” refers to a gene encoding a RASGEF1A mRNA or polypeptide. The RASGEF1A gene encodes the RasGEF domain family member 1A protein. RASGEF1A is also known as CG4853. In some embodiments, RASGEF1A gene is a human RASGEF1A. An exemplary RASGEF1A gene is represented by NCBI Gene ID No. 221002. An exemplary RASGEF1A mRNA sequence is represented by NCBI Ref. Seq. NM_145313. An exemplary amino acid sequence of a RASGEF1A polypeptide is represented by NCBI Ref. Seq. NP_660356.

As used herein “CCDC6” refers to a gene encoding a CCDC6 mRNA or polypeptide. The CCDC6 gene encodes the coiled-coil domain containing 6 protein. CCDC6 is also known as D10S170, H4, PTC, TPC, and TST1. In some embodiments, CCDC6 gene is a human CCDC6. An exemplary CCDC6 gene is represented by NCBI Gene ID No. 8030. An exemplary CCDC6 mRNA sequence is represented by NCBI Ref. Seq. NM_005436. An exemplary amino acid sequence of a CCDC6 polypeptide is represented by NCBI Ref. Seq. NP_005427.

As used herein “ERC1” refers to a gene encoding an ERC1 mRNA or polypeptide. The ERC1 gene encodes the coiled-coil domain containing 6 protein. ERC1 is also known as Cast2, ELKS, ERC-1, and RAB6IP2. In some embodiments, ERC1 gene is a human ERC1. An exemplary ERC1 gene is represented by NCBI Gene ID No. 23085. An exemplary ERC1 mRNA sequence is represented by NCBI Ref. Seq. NM_178039. An exemplary amino acid sequence of an ERC1 polypeptide is represented by NCBI Ref. Seq. NP_829883.

As used herein “KIAA1217” refers to a gene encoding a KIAA1217 mRNA or polypeptide. The KIAA1217 gene encodes the KIAA1217 protein. KIAA1217 is also known as ETL4 and SKT. In some embodiments, KIAA1217 gene is a human KIAA1217. An exemplary KIAA1217 gene is represented by NCBI Gene ID No. 56243. An exemplary KIAA1217 mRNA sequence is represented by NCBI Ref. Seq. NM_019590. An exemplary amino acid sequence of a KIAA1217 polypeptide is represented by NCBI Ref. Seq. NP_062536.

As used herein “KIF5B” refers to a gene encoding a KIF5B mRNA or polypeptide. The KIF5B gene encodes the kinesin family member 5B protein. KIF5B is also known as HEL-S-61, KINH, KNS, KNS1, and UKHC. In some embodiments, KIF5B gene is a human KIF5B. An exemplary KIF5B gene is represented by NCBI Gene ID No. 3799. An exemplary KIF5B mRNA sequence is represented by NCBI Ref. Seq. NM_004521. An exemplary amino acid sequence of a KIF5B polypeptide is represented by NCBI Ref. Seq. NP_004512.

As used herein “TRIM24” refers to a gene encoding a TRIM24 mRNA or polypeptide. The TRIM24 gene encodes the tripartite motif containing 24 protein. TRIM24 is also known as PTC6, TF1A, TIF1, RNF82, TIF1A, hTIF1, and TIF1ALPHA. In some embodiments, a TRIM24 gene is a human TRIM24 gene. An exemplary TRIM24 gene is represented by NCBI Gene ID No. 8805. An exemplary TRIM24 mRNA sequence is represented by NCBI Ref. Seq. NM_003852. An exemplary amino acid sequence of a TRIM24 polypeptide is represented by NCBI Ref. Seq. NP_003843.

As used herein “VCL” refers to a gene encoding a VCL mRNA or polypeptide. The VCL gene encodes the vinculin protein. VCL is also known as CMD1W, CMH15, HEL114, MV, and MVCL. In some embodiments, a VCL gene is a human VCL gene. An exemplary VCL gene is represented by NCBI Gene ID No. 7414. An exemplary VCL mRNA sequence is represented by NCBI Ref. Seq. NM_003373. An exemplary amino acid sequence of a VCL polypeptide is represented by NCBI Ref. Seq. NP_003364.

In some aspects, provided herein are ROS1 fusion nucleic acid molecules comprising at least a portion of ROS1 and at least a portion of another gene.

In some embodiments, a ROS1 fusion nucleic acid molecule comprises at least a portion of ROS1 and at least a portion of ABR, ASCC3, ELOVL4, QKI, REV3L, MED23, SLC30A8, SLC38A11, TLN1, SLC26A2, SYNGR1, EZR, GOPC, MYO5C, TPD52L1, or TRPC6. For example, in some embodiments, the ROS1 fusion nucleic acid molecule is selected from ROS1-ABR, ROS1-ASCC3, ROS1-ELOVL4, ROS1-QKI, ROS1-REV3L, MED23-ROS1, SLC30A8-ROS1, SLC38A11-ROS1, TLN1-ROS1, ROS1-SLC26A2, ROS1-SYNGR1, ROS1-TRPC6, EZR-ROS1, GOPC-ROS1, MYO5C-ROS1, or ROS1-TPD52L1, wherein the order of the genes is in the 5′ to 3′ direction. Exemplary and non-limiting ROS1 fusion nucleic acid molecules are described herein and/or in Tables 1-6, and/or in the Examples herein.

As used herein “ABR” refers to a gene encoding an ABR mRNA or polypeptide. The ABR gene encodes the ABR activator of RhoGEF and GTPase protein. ABR is also known as MDB. In some embodiments, an ABR gene is a human ABR gene. An exemplary ABR gene is represented by NCBI Gene ID No. 29. An exemplary ABR mRNA sequence is represented by NCBI Ref. Seq. NM_001092. An exemplary amino acid sequence of an ABR polypeptide is represented by NCBI Ref. Seq. NP_001083.

As used herein “ASCC3” refers to a gene encoding an ASCC3 mRNA or polypeptide. The ASCC3 gene encodes the activating signal cointegrator 1 complex subunit 3 protein. ASCC3 is also known as ASC1p200, HELIC1, and RNAH. In some embodiments, an ASCC3 gene is a human ASCC3 gene. An exemplary ASCC3 gene is represented by NCBI Gene ID No. 10973. An exemplary ASCC3 mRNA sequence is represented by NCBI Ref. Seq. NM_006828. An exemplary amino acid sequence of an ASCC3 polypeptide is represented by NCBI Ref. Seq. NP_006819.

As used herein “ELOVL4” refers to a gene encoding an ELOVL4 mRNA or polypeptide. The ELOVL4 gene encodes the ELOVL fatty acid elongase 4 protein. ELOVL4 is also known as ADMD, CT118, ISQMR, SCA34, STGD2, and STGD3. In some embodiments, an ELOVL4 gene is a human ELOVL4 gene. An exemplary ELOVL4 gene is represented by NCBI Gene ID No. 6785. An exemplary ELOVL4 mRNA sequence is represented by NCBI Ref. Seq. NM_022726. An exemplary amino acid sequence of an ELOVL4 polypeptide is represented by NCBI Ref. Seq. NP_073563.

As used herein “QKI” refers to a gene encoding a QKI mRNA or polypeptide. The QKI gene encodes the QKI, KH domain containing RNA binding protein. QKI is also known as Hqk, QK, QK1, QK3, and hqkI. In some embodiments, a QKI gene is a human QKI gene. An exemplary QKI gene is represented by NCBI Gene ID No. 9444. An exemplary QKI mRNA sequence is represented by NCBI Ref. Seq. NM_006775. An exemplary amino acid sequence of a QKI polypeptide is represented by NCBI Ref. Seq. NP_006766.

As used herein “REV3L” refers to a gene encoding a REV3L mRNA or polypeptide. The REV3L gene encodes the REV3 like, DNA directed polymerase zeta catalytic subunit protein. REV3L is also known as POLZ and REV3. In some embodiments, a REV3L gene is a human REV3L gene. An exemplary REV3L gene is represented by NCBI Gene ID No. 5980. An exemplary REV3L mRNA sequence is represented by NCBI Ref. Seq. NM_002912. An exemplary amino acid sequence of a REV3L polypeptide is represented by NCBI Ref. Seq. NP_002903.

As used herein “MED23” refers to a gene encoding a MED23 mRNA or polypeptide. The MED23 gene encodes the MED23 like, DNA directed polymerase zeta catalytic subunit protein. MED23 is also known as ARC130, CRSP130, CRSP133, CRSP3, DRIP130, MRT18, SUR-2, and SUR2. In some embodiments, a MED23 gene is a human MED23 gene. An exemplary MED23 gene is represented by NCBI Gene ID No. 9439. An exemplary MED23 mRNA sequence is represented by NCBI Ref. Seq. NM_004830. An exemplary amino acid sequence of a MED23 polypeptide is represented by NCBI Ref. Seq. NP_004821.

As used herein “SLC30A8” refers to a gene encoding a SLC30A8 mRNA or polypeptide. The SLC30A8 gene encodes the solute carrier family 30 member 8 protein. SLC30A8 is also known as ZNT8 and ZnT-8. In some embodiments, a SLC30A8 gene is a human SLC30A8 gene. An exemplary SLC30A8 gene is represented by NCBI Gene ID No. 169026. An exemplary SLC30A8 mRNA sequence is represented by NCBI Ref. Seq. NM_001172811. An exemplary amino acid sequence of a SLC30A8 polypeptide is represented by NCBI Ref. Seq. NP_001166282.

As used herein “SLC38A11” refers to a gene encoding a SLC38A11 mRNA or polypeptide. The SLC38A11 gene encodes the solute carrier family 38 member 11 protein. SLC38A11 is also known as AVT2. In some embodiments, a SLC38A11 gene is a human SLC38A11 gene. An exemplary SLC38A11 gene is represented by NCBI Gene ID No. 151258. An exemplary SLC38A11 mRNA sequence is represented by NCBI Ref. Seq. NM_173512. An exemplary amino acid sequence of a SLC38A11 polypeptide is represented by NCBI Ref. Seq. NP_775783.

As used herein “TLN1” refers to a gene encoding a TLN1 mRNA or polypeptide. The TLN1 gene encodes talin 1 protein. TLN1 is also known as ILWEQ, TLN, and talin-1. In some embodiments, a TLN1 gene is a human TLN1 gene. An exemplary TLN1 gene is represented by NCBI Gene ID No. 7094. An exemplary TLN1 mRNA sequence is represented by NCBI Ref. Seq. NM_006289. An exemplary amino acid sequence of a TLN1 polypeptide is represented by NCBI Ref. Seq. NP_006280.

As used herein “SLC26A2” refers to a gene encoding a SLC26A2 mRNA or polypeptide. The SLC26A2 gene encodes the solute carrier family 26 member 2 protein. SLC26A2 is also known as D5S1708, DTD, DTDST, EDM4, MST153, and MSTP157. In some embodiments, a SLC26A2 gene is a human SLC26A2 gene. An exemplary SLC26A2 gene is represented by NCBI Gene ID No. 1836. An exemplary SLC26A2 mRNA sequence is represented by NCBI Ref. Seq. NM_000112. An exemplary amino acid sequence of a SLC26A2 polypeptide is represented by NCBI Ref. Seq. NP_000103.

As used herein “SYNGR1” refers to a gene encoding a SYNGR1 mRNA or polypeptide. The SYNGR1 gene encodes the synaptogyrin 1 protein. In some embodiments, a SYNGR1 gene is a human SYNGR1 gene. An exemplary SYNGR1 gene is represented by NCBI Gene ID No. 9145. An exemplary SYNGR1 mRNA sequence is represented by NCBI Ref. Seq. NM_004711. An exemplary amino acid sequence of a SYNGR1 polypeptide is represented by NCBI Ref. Seq. NP_004702.

As used herein “EZR” refers to a gene encoding an EZR mRNA or polypeptide. The EZR gene encodes the synaptogyrin 1 protein. EZR is also known as CVIL, CVL, HEL-S-105, and VIL2. In some embodiments, an EZR gene is a human EZR gene. An exemplary EZR gene is represented by NCBI Gene ID No. 7430. An exemplary EZR mRNA sequence is represented by NCBI Ref. Seq. NM_003379. An exemplary amino acid sequence of an EZR polypeptide is represented by NCBI Ref. Seq. NP_003370.

As used herein “GOPC” refers to a gene encoding a GOPC mRNA or polypeptide. The GOPC gene encodes the golgi associated PDZ and coiled-coil motif containing protein. GOPC is also known as CAL, FIG, GOPC1, PIST, and dJ94G16.2. In some embodiments, a GOPC gene is a human GOPC gene. An exemplary GOPC gene is represented by NCBI Gene ID No. 57120. An exemplary GOPC mRNA sequence is represented by NCBI Ref. Seq. NM_020399. An exemplary amino acid sequence of a GOPC polypeptide is represented by NCBI Ref. Seq. NP_065132.

As used herein “MYO5C” refers to a gene encoding a MYO5C mRNA or polypeptide. The MYO5C gene encodes the myosin VC protein. In some embodiments, a MYO5C gene is a human MYO5C gene. An exemplary MYO5C gene is represented by NCBI Gene ID No. 55930. An exemplary MYO5C mRNA sequence is represented by NCBI Ref. Seq. NM_018728. An exemplary amino acid sequence of a MYO5C polypeptide is represented by NCBI Ref. Seq. NP_061198.

As used herein “TPD52L1” refers to a gene encoding a TPD52L1 mRNA or polypeptide. The TPD52L1 gene encodes the TPD52 like 1 protein. TPD52L1 is also known as D53 and TPD53. In some embodiments, a TPD52L1 gene is a human TPD52L1 gene. An exemplary TPD52L1 gene is represented by NCBI Gene ID No. 7164. An exemplary TPD52L1 mRNA sequence is represented by NCBI Ref. Seq. NM_003287. An exemplary amino acid sequence of a TPD52L1 polypeptide is represented by NCBI Ref. Seq. NP_003278.

As used herein “TRPC6” refers to a gene encoding a TRPC6 mRNA or polypeptide. The TRPC6 gene encodes the transient receptor potential cation channel subfamily C member 6 protein. TRPC6 is also known as FSGS2 and TRP6. In some embodiments, a TRPC6 gene is a human TRPC6 gene. An exemplary TRPC6 gene is represented by NCBI Gene ID No. 7225. An exemplary TRPC6 mRNA sequence is represented by NCBI Ref. Seq. NM_004621. An exemplary amino acid sequence of a TRPC6 polypeptide is represented by NCBI Ref. Seq. NP_004612.

Exemplary ALK, BRAF, EGFR, ERBB2, FGFR1, FGFR2, FGFR3, MET, NTRK1, RAF1, RET, and ROS1 fusion nucleic acid molecules are provided in Tables 1 and 2, below.

TABLE 1
Exemplary ALK, BRAF, EGFR, ERBB2, FGFR1, FGFR2, FGFR3,
MET, RAF1, RET, and ROS1 fusion nucleic acid molecules.

| ALK Fusion Nucleic Acid Molecule | 5′ Gene | 3′ Gene |
|---|---|---|
| ALK-AGAP1 | AGAP1 | ALK |
| ALK-ARHGEF7 | ARHGEF7 | ALK |
| ALK-BRE | BRE | ALK |
| ALK-EPS8 | EPS8 | ALK |
| ALK-GPR113 | GPR113 | ALK |
| ALK-HDAC9 | HDAC9 | ALK |
| ALK-MIPOL1 | MIPOL1 | ALK |
| ALK-PELI1 | PELI1 | ALK |
| ALK-SLC39A10 | SLC39A10 | ALK |
| ALK-VKORCIL1 | VKORC1L1 | ALK |
| ALK-SORBS1 | ALK | SORBS1 |
| ALK-SPINK5 | ALK | SPINK5 |
| BRAF Fusion Nucleic Acid Molecule | 5′ Gene | 3′ Gene |
| BRAF-CCDC88C | CCDC88C | BRAF |
| BRAF-COBLL1 | COBLL1 | BRAF |
| BRAF-CREB3L2 | CREB3L2 | BRAF |
| BRAF-DLC1 | DLC1 | BRAF |
| BRAF-GOLGA3 | GOLGA3 | BRAF |
| BRAF-MSI2 | MSI2 | BRAF |
| BRAF-TNS3 | TNS3 | BRAF |
| BRAF-DOCK4 | BRAF | DOCK4 |
| BRAF-RAD51 | BRAF | RAD51 |
| EGFR Fusion Nucleic Acid Molecule | 5′ Gene | 3′ Gene |
| EGFR-ABCB1 | ABCB1 | EGFR |
| EGFR-PDE7A | PDE7A | EGFR |
| EGFR-EZH2 | EGFR | EZH2 |
| EGFR-FLJ45974 | EGFR | FLJ45974 |
| EGFR-ZNF479 | EGFR | ZNF479 |
| ERBB2 Fusion Nucleic Acid Molecule | 5′ Gene | 3′ Gene |
| ERBB2-FBXL20 | FBXL20 | ERBB2 |
| ERBB2-GRB7 | GRB7 | ERBB2 |
| ERBB2-MSI2 | MSI2 | ERBB2 |
| ERBB2-RANBP10 | RANBP10 | ERBB2 |
| ERBB2-SEC14L1 | SEC14L1 | ERBB2 |
| ERBB2-WIPF2 | WIPF2 | ERBB2 |
| ERBB2-GRB7 | ERBB2 | GRB7 |
| ERBB2-PRKCA | ERBB2 | PRKCA |
| FGFR1 Fusion Nucleic Acid Molecule | 5′ Gene | 3′ Gene |
| FGFR1-ADAM32 | FGFR1 | ADAM32 |
| FGFR1-SLC12A8 | FGFR1 | SLC12A8 |
| FGFR2 Fusion Nucleic Acid Molecule | 5′ Gene | 3′ Gene |
| FGFR2-AARSD1 | FGFR2 | AARSD1 |
| FGFR2-ARMS2 | FGFR2 | ARMS2 |
| FGFR2-ATF7 | FGFR2 | ATF7 |
| FGFR2-BAIAP2L1 | FGFR2 | BAIAP2L1 |
| FGFR2-CCAR1 | FGFR2 | CCAR1 |
| FGFR2-CCSER2 | FGFR2 | CCSER2 |
| FGFR2-CGNL1 | FGFR2 | CGNL1 |
| FGFR2-EBF1 | FGFR2 | EBF1 |
| FGFR2-FANK1 | FGFR2 | FANK1 |
| FGFR2-FOXP1 | FGFR2 | FOXP1 |
| FGFR2-CAMK2G | CAMK2G | FGFR2 |
| FGFR2-FLJ40288 | FLJ40288 | FGFR2 |
| FGFR2-GUCY2D | GUCY2D | FGFR2 |
| FGFR2-IQGAP2 | IQGAP2 | FGFR2 |
| FGFR2-PAWR | PAWR | FGFR2 |
| FGFR2-FLNB | FGFR2 | FLNB |
| FGFR2-FOXP1 | FGFR2 | FOXP1 |
| FGFR2-IKZF2 | FGFR2 | IKZF2 |
| FGFR2-KHDRBS1 | FGFR2 | KHDRBS1 |
| FGFR2-MYOZ1 | FGFR2 | MYOZ1 |
| FGFR2-PCDH15 | FGFR2 | PCDH15 |
| FGFR2-PRKAR1A | FGFR2 | PRKAR1A |
| FGFR2-PRRC2A | FGFR2 | PRRC2A |
| FGFR2-RABGAP1 | FGFR2 | RABGAP1 |
| FGFR2-SCIN | FGFR2 | SCIN |
| FGFR2-STAU1 | FGFR2 | STAU1 |
| FGFR2-STK4 | FGFR2 | STK4 |
| FGFR2-TIFA | FGFR2 | TIFA |
| FGFR2-TLK1 | FGFR2 | TLK1 |
| FGFR2-TRIM54 | FGFR2 | TRIM54 |
| FGFR3 Fusion Nucleic Acid Molecule | 5′ Gene | 3′ Gene |
| FGFR3-CCT5 | FGFR3 | CCT5 |
| FGFR3-CNOT4 | FGFR3 | CNOT4 |
| FGFR3-TNIP2 | FGFR3 | TNIP2 |
| MET Fusion Nucleic Acid Molecule | 5′ Gene | 3′ Gene |
| MET-LDHA | MET | LDHA |
| MET-CNTNAP2 | CNTNAP2 | MET |
| MET-HBP1 | HBP1 | MET |
| MET-SNRNP70 | SNRNP70 | MET |
| RAF1 Fusion Nucleic Acid Molecule | 5′ Gene | 3′ Gene |
| RAF1-POC1A | POC1A | RAF1 |
| RAF1-SYN2 | SYN2 | RAF1 |
| RAF1-ZFYVE20 | ZFYVE20 | RAF1 |
| RET Fusion Nucleic Acid Molecule | 5′ Gene | 3′ Gene |
| RET-ADCY1 | RET | ADCY1 |
| RET-BAIAP2L1 | BAIAP2L1 | RET |
| RET-NPY4R | RET | NPY4R |
| RET-PAWR | RET | PAWR |
| RET-ALOX5 | ALOX5 | RET |
| RET-ARID5B | ARID5B | RET |
| RET-DHX32 | DHX32 | RET |
| RET-PDE5A | PDE5A | RET |
| RET-ZNF365 | ZNF365 | RET |
| ROS1 Fusion Nucleic Acid Molecule | 5′ Gene | 3′ Gene |
| ROS1-ABR | ROS1 | ABR |
| ROS1-ASCC3 | ROS1 | ASCC3 |
| ROS1-ELOVL4 | ROS1 | ELOVL4 |
| ROS1-QKI | ROS1 | QKI |
| ROS1-REV3L | ROS1 | REV3L |
| ROS1-MED23 | MED23 | ROS1 |
| ROS1-SLC30A8 | SLC30A8 | ROS1 |
| ROS1-SLC38A11 | SLC38A11 | ROS1 |
| ROS1-TLN1 | TLN1 | ROS1 |
| ROS1-SLC26A2 | ROS1 | SLC26A2 |
| ROS1-SYNGR1 | ROS1 | SYNGR1 |
| ROS1-TRPC6 | ROS1 | TRPC6 |

TABLE 2
Exemplary ALK, BRAF, ERBB2, FGFR1, FGFR2, FGFR3, MET, NTRK1, RAF1,
RET, and ROS1 fusion nucleic acid molecules identified in the indicated cancers.

| ALK Fusion | | | |
|---|---|---|---|
| Nucleic Acid | | | |
| Molecule | Cancer | 5′ Gene | 3′ Gene |
| ALK-GCC2 | lung squamous cell carcinoma (SCC) | GCC2 | ALK |
| ALK-HIP1 | unknown primary carcinoma (CUP) (NOS) | HIP1 | ALK |
| ALK | pancreas (NOS) | KANK1 | ALK |
| KANK1 | | | |
| ALK-KLC1 | unknown primary adenocarcinoma | KLC1 | ALK |
| ALK | unknown primary (NOS) | PPFIBP1 | ALK |
| PPFIBP1 | | | |
| ALK | lung non-small cell lung carcinoma (NSCLC) | PLEKHA7 | ALK |
| PLEKHA7 | (NOS) | | |
| ALK-TFG | breast carcinoma (NOS) | TFG | ALK |
| ALK-TPM3 | pancreas ductal adenocarcinoma | TPM3 | ALK |
| BRAF Fusion | | | |
| Nucleic Acid | | | |
| Molecule | Cancer | 5′ Gene | 3′ Gene |
| BRAF- | colon adenocarcinoma (CRC) | AKAP9 | BRAF |
| AKAP9 | | | |
| BRAF- | esophagus adenocarcinoma | ARMC10 | BRAF |
| ARMC10 | | | |
| BRAF- | colon adenocarcinoma (CRC) | DENND2A | BRAF |
| DENND2A | | | |
| BRAF- | prostate acinar adenocarcinoma | JHDM1D | BRAF |
| JHDM1D | | | |
| BRAF- | prostate acinar adenocarcinoma | KIAA1549 | BRAF |
| KIAA1549 | | | |
| BRAF- | rectum adenocarcinoma (CRC) | MKRN1 | BRAF |
| MKRN1 | | | |
| BRAF-NRF1 | colon adenocarcinoma (CRC) | NRF1 | BRAF |
| BRAF- | prostate acinar adenocarcinoma | SLC45A3 | BRAF |
| SLC45A3 | | | |
| BRAF-SND1 | prostate acinar adenocarcinoma | SND1 | BRAF |
| | prostate ductal adenocarcinoma | | |
| | colon adenocarcinoma (CRC) | | |
| BRAF- | rectum adenocarcinoma (CRC) | BRAF | TRIM24 |
| TRIM24 | | | |
| BRAF- | colon adenocarcinoma (CRC) | ZC3HAV1 | BRAF |
| ZC3HAV1 | | | |
| BRAF- | lung adenocarcinoma | ZNF277 | BRAF |
| ZNF277 | | | |
| ERBB2 | | | |
| Fusion | | | |
| Nucleic Acid | | | |
| Molecule | Cancer | 5′ Gene | 3′ Gene |
| ERBB2- | colon adenocarcinoma (CRC) | ERBB2 | PPP1R1B |
| PPP1R1B | | | |
| FGFR1 | | | |
| Fusion | | | |
| Nucleic Acid | | | |
| Molecule | Cancer | 5′ Gene | 3′ Gene |
| FGFR1- | cervix squamous cell carcinoma (SCC) | ADAM18 | FGFR1 |
| ADAM18 | | | |
| FGFR1 | breast (NOS) | BAG4 | FGFR1 |
| BAG4 | | | |
| FGFR1- | breast invasive lobular carcinoma (ILC) | FGFR1 | TACC1 |
| TACC1 | | | |
| FGFR2 | | | |
| Fusion | | | |
| Nucleic Acid | | | |
| Molecule | Cancer | 5′ Gene | 3′ Gene |
| FGFR2-APIP | lung large cell carcinoma | FGFR2 | APIP |
| FGFR2- | stomach adenocarcinoma (NOS) | FGFR2 | ATE1 |
| ATE1 | | | |
| FGFR2- | unknown primary carcinoma (CUP) (NOS) | FGFR2 | BICC1 |
| BICC1 | pancreatobiliary carcinoma | | |
| | lung adenocarcinoma | | |
| FGFR2 | prostate (NOS) | TFEC | FGFR2 |
| TFEC | | | |
| FGFR2 | intra-hepatic cholangiocarcinoma | FGFR2 | GRB2 |
| GRB2 | breast invasive ductal carcinoma (IDC) | | |
| FGFR2- | gallbladder adenocarcinoma | FGFR2 | KIAA1217 |
| KIAA1217 | | | |
| FGFR2- | lung adenocarcinoma | FGFR2 | KIAA1598 |
| KIAA1598 | | | |
| FGFR2- | pancreatobiliary carcinoma | FGFR2 | MACF1 |
| MACF1 | | | |
| FGFR2- | unknown primary adenocarcinoma | FGFR2 | MYH9 |
| MYH9 | | | |
| FGFR2 | lung squamous cell carcinoma (SCC) | FGFR2 | NRAP |
| NRAP | | | |
| FGFR2 | breast carcinoma (NOS) | FGFR2 | RBM20 |
| RBM20 | | | |
| FGFR2- | intra-hepatic cholangiocarcinoma | FGFR2 | SPICE1 |
| SPICE1 | | | |
| FGFR2 | pancreatobiliary carcinoma | FGFR2 | TACC2 |
| TACC2 | unknown primary adenocarcinoma | | |
| | lung small cell undifferentiated carcinoma | | |
| | breast invasive ductal carcinoma (IDC) | | |
| FGFR2- | stomach adenocarcinoma (NOS) | FGFR2 | VTI1A |
| VTI1A | | | |
| FGFR2- | lung adenocarcinoma | FGFR2 | WAC |
| WAC | | | |
| FGFR2- | intra-hepatic cholangiocarcinoma | FGFR2 | WARS |
| WARS | lung non-small cell lung carcinoma (NSCLC) | | |
| | (NOS) | | |
| FGFR2- | unknown primary carcinoma (CUP) (NOS) | FGFR2 | ZMYM4 |
| ZMYM4 | | | |
| FGFR3 | | | |
| Fusion | | | |
| Nucleic Acid | | | |
| Molecule | Cancer | 5′ Gene | 3′ Gene |
| FGFR3- | lung non-small cell lung carcinoma (NSCLC) | FGFR3 | ADD1 |
| ADD1 | (NOS) | | |
| FGFR3-IGH | pancreas (NOS) | FGFR3 | IGH |
| FGFR3- | small intestine adenocarcinoma | FGFR3 | TACC3 |
| TACC3 | prostate acinar adenocarcinoma | | |
| | colon adenocarcinoma (CRC) | | |
| | unknown primary squamous cell carcinoma | | |
| | (SCC) | | |
| | breast invasive lobular carcinoma (ILC) | | |
| | eye intraocular melanoma | | |
| | bladder adenocarcinoma | | |
| | lung small cell undifferentiated carcinoma | | |
| | kidney (NOS) | | |
| FGFR3- | prostate acinar adenocarcinoma | FGFR3 | WHSC1 |
| WHSC1 | | | |
| MET Fusion | | | |
| Nucleic Acid | | | |
| Molecule | Cancer | 5′ Gene | 3′ Gene |
| MET- | colon adenocarcinoma (CRC) | MET | CAPZA2 |
| CAPZA2 | | | |
| MET-ST7 | colon adenocarcinoma (CRC) | ST7 | MET |
| | skin melanoma | | |
| NTRK1 | Cancer | 5′ Gene | 3′ Gene |
| Fusion | | | |
| Nucleic Acid | | | |
| Molecule | | | |
| NTRK1 | prostate (NOS) | NTRK1 | MEF2D |
| MEF2D | | | |
| RAF1 Fusion | | | |
| Nucleic Acid | | | |
| Molecule | Cancer | 5′ Gene | 3′ Gene |
| RAF1 | colon adenocarcinoma (CRC) | RAF1 | TRAK1 |
| TRAK1 | | | |
| RET Fusion | | | |
| Nucleic Acid | | | |
| Molecule | Cancer | 5′ Gene | 3′ Gene |
| RET- | esophagus squamous cell carcinoma (SCC) | RET | CSGALNACT2 |
| CSGALNACT2 | | | |
| RET-GPHN | lung non-small cell lung carcinoma (NSCLC) | RET | GPHN |
| | (NOS) | | |
| RET- | bladder urothelial (transitional cell) carcinoma | NCOA4 | RET |
| NCOA4 | unknown primary carcinoma (CUP) (NOS) | | |
| RET- | colon adenocarcinoma (CRC) | RET | RASGEF1A |
| RASGEF1A | | | |
| RET-CCDC6 | colon adenocarcinoma (CRC) | CCDC6 | RET |
| | rectum adenocarcinoma (CRC) | | |
| RET-ERC1 | soft tissue sarcoma (NOS) | ERC1 | RET |
| RET- | colon adenocarcinoma (CRC) | KIAA1217 | RET |
| KIAA1217 | esophagus adenocarcinoma | | |
| | lung non-small cell lung carcinoma (NSCLC) | | |
| | (NOS) | | |
| RET-KIF5B | breast (NOS) | KIF5B | RET |
| | lung squamous cell carcinoma (SCC) | | |
| | unknown primary adenocarcinoma | | |
| RET- | lung adenocarcinoma | TRIM24 | RET |
| TRIM24 | | | |
| RET-VCL | lung adenocarcinoma | VCL | RET |
| ROS1 Fusion | | | |
| Nucleic Acid | | | |
| Molecule | Cancer | 5′ Gene | 3′ Gene |
| ROS1-EZR | unknown primary carcinoma (CUP) (NOS) | EZR | ROS1 |
| ROS1-GOPC | appendix adenocarcinoma | GOPC | ROS1 |
| | liver hepatocellular carcinoma (HCC) | | |
| | rectum adenocarcinoma (CRC) | | |
| | unknown primary neuroendocrine tumor | | |
| ROS1- | lung adenocarcinoma | MYO5C | ROS1 |
| MYO5C | | | |
| ROS1- | prostate acinar adenocarcinoma | ROS1 | TPD52L1 |
| TPD52L1 | | | |

SCC: squamous cell carcinoma;
CUP: carcinoma of unknown primary;
NOS: not otherwise specified;
NSCLC: non-small cell lung cancer;
CRC: colorectal cancer;
ILC: invasive lobular breast cancer;
IDC: invasive ductal carcinoma;
HCC: hepatocellular carcinoma

In some aspects, provided herein are ALK fusion nucleic acid molecules comprising at least a portion of ALK and at least a portion of another gene. In some embodiments, an ALK fusion nucleic acid molecule provided herein is an AGAP1-ALK fusion nucleic acid molecule, in the 5′ to 3′ direction, comprising or resulting from a breakpoint within chromosomal coordinates chr2:29446320 and/or chr2:236984410. In some embodiments, an ALK fusion nucleic acid molecule provided herein is an ARHGEF7-ALK fusion nucleic acid molecule, in the 5′ to 3′ direction, comprising or resulting from a breakpoint within chromosomal coordinates chr2:29448192 and/or chr13:111796690. In some embodiments, an ALK fusion nucleic acid molecule provided herein is a BRE-ALK fusion nucleic acid molecule, in the 5′ to 3′ direction, comprising or resulting from a breakpoint within chromosomal coordinates chr2:29448094-29448239 and/or chr2:28431656-28431790. In some embodiments, an ALK fusion nucleic acid molecule provided herein is an EPS8-ALK fusion nucleic acid molecule, in the 5′ to 3′ direction, comprising or resulting from a breakpoint within chromosomal coordinates chr2:29448033 and/or chr12:15811779. In some embodiments, an ALK fusion nucleic acid molecule provided herein is a GPR113-ALK fusion nucleic acid molecule, in the 5′ to 3′ direction, comprising or resulting from a breakpoint within chromosomal coordinates chr2:29448344 and/or chr2:26546664. In some embodiments, an ALK fusion nucleic acid molecule provided herein is an HDAC9-ALK fusion nucleic acid molecule, in the 5′ to 3′ direction, comprising or resulting from a breakpoint within chromosomal coordinates chr2:29420593 and/or chr7:18908783. In some embodiments, an ALK fusion nucleic acid molecule provided herein is a MIPOL1-ALK fusion nucleic acid molecule, in the 5′ to 3′ direction, comprising or resulting from a breakpoint within chromosomal coordinates chr2:29446669 and/or chr14:37906366. In some embodiments, an ALK fusion nucleic acid molecule provided herein is an PELI1-ALK fusion nucleic acid molecule, in the 5′ to 3′ direction, comprising or resulting from a breakpoint within chromosomal coordinates chr2:29448116 and/or chr2:64360629. In some embodiments, an ALK fusion nucleic acid molecule provided herein is an SLC39A10-ALK fusion nucleic acid molecule, in the 5′ to 3′ direction, comprising or resulting from a breakpoint within chromosomal coordinates chr2:29448047 and/or chr2:196583393. In some embodiments, an ALK fusion nucleic acid molecule provided herein is an VKORC1L1-ALK fusion nucleic acid molecule, in the 5′ to 3′ direction, comprising or resulting from a breakpoint within chromosomal coordinates chr2:29447360-29447477 and/or chr7:65386539-65386741. In some embodiments, an ALK fusion nucleic acid molecule provided herein is an ALK-SORBS1 fusion nucleic acid molecule, in the 5′ to 3′ direction, comprising or resulting from a breakpoint within chromosomal coordinates chr2:29447840 and/or chr10:97287128. In some embodiments, an ALK fusion nucleic acid molecule provided herein is an ALK-SPINK5 fusion nucleic acid molecule, in the 5′ to 3′ direction, comprising or resulting from a breakpoint within chromosomal coordinates chr2:29448698 and/or chr5:147513014. In some embodiments, an ALK fusion nucleic acid molecule provided herein is a GCC2-ALK fusion nucleic acid molecule, in the 5′ to 3′ direction, comprising or resulting from a breakpoint within chromosomal coordinates chr2:29447157 and/or chr2:109110640. In some embodiments, an ALK fusion nucleic acid molecule provided herein is a HIP1-ALK fusion nucleic acid molecule, in the 5′ to 3′ direction, comprising or resulting from a breakpoint within chromosomal coordinates chr2:29447938-29448138 and/or chr7:75171619-75171750. In some embodiments, an ALK fusion nucleic acid molecule provided herein is a KANK1-ALK fusion nucleic acid molecule, in the 5′ to 3′ direction, comprising or resulting from a breakpoint within chromosomal coordinates chr2:29449486-29449674 and/or chr9:723093-723285. In some embodiments, an ALK fusion nucleic acid molecule provided herein is a KLC1-ALK fusion nucleic acid molecule, in the 5′ to 3′ direction, comprising or resulting from a breakpoint within chromosomal coordinates chr2:29448020-29448230 and/or chr14:104141458-104141626. In some embodiments, an ALK fusion nucleic acid molecule provided herein is a PPFIBP1-ALK fusion nucleic acid molecule, in the 5′ to 3′ direction, comprising or resulting from a breakpoint within chromosomal coordinates chr2:29447358-29447625 and/or chr12:27813429-27813685. In some embodiments, an ALK fusion nucleic acid molecule provided herein is a PLEKHA7-ALK fusion nucleic acid molecule, in the 5′ to 3′ direction, comprising or resulting from a breakpoint within chromosomal coordinates chr2:29448437-29448765 and/or chr11:16803216-16803516. In some embodiments, an ALK fusion nucleic acid molecule provided herein is a TFG-ALK fusion nucleic acid molecule, in the 5′ to 3′ direction, comprising or resulting from a breakpoint within chromosomal coordinates chr2:29449097 and/or chr3:100450538. In some embodiments, an ALK fusion nucleic acid molecule provided herein is a TPM3-ALK fusion nucleic acid molecule, in the 5′ to 3′ direction, comprising or resulting from a breakpoint within chromosomal coordinates chr2:29448196 and/or chr1:154135477. In some embodiments, an ALK fusion nucleic acid molecule provided herein is an ALK fusion nucleic acid molecule listed in any of Tables 3, 5, or 6, comprising or resulting from a Breakpoint 1 and/or Breakpoint 2 within the corresponding chromosomal coordinates as listed in any of Tables 3, 5, or 6.

In some aspects, provided herein are BRAF fusion nucleic acid molecules comprising at least a portion of BRAF and at least a portion of another gene. In some embodiments, a BRAF fusion nucleic acid molecule provided herein is a CCDC88C-BRAF fusion nucleic acid molecule, in the 5′ to 3′ direction, comprising or resulting from a breakpoint within chromosomal coordinates chr7:140489237 and/or chr14:91742455. In some embodiments, a BRAF fusion nucleic acid molecule provided herein is a COBLL1-BRAF fusion nucleic acid molecule, in the 5′ to 3′ direction, comprising or resulting from a breakpoint within chromosomal coordinates chr7:140489195 and/or chr2:165542493. In some embodiments, a BRAF fusion nucleic acid molecule provided herein is a CREB3L2-BRAF fusion nucleic acid molecule, in the 5′ to 3′ direction, comprising or resulting from a breakpoint within chromosomal coordinates chr7:140482515 and/or chr7:137655487. In some embodiments, a BRAF fusion nucleic acid molecule provided herein is a DLC1-BRAF fusion nucleic acid molecule, in the 5′ to 3′ direction, comprising or resulting from a breakpoint within chromosomal coordinates chr7:140434573 and/or chr8:13242758. In some embodiments, a BRAF fusion nucleic acid molecule provided herein is a GOLGA3-BRAF fusion nucleic acid molecule, in the 5′ to 3′ direction, comprising or resulting from a breakpoint within chromosomal coordinates chr7:140494157 and/or chr12:133360988. In some embodiments, a BRAF fusion nucleic acid molecule provided herein is an MSI2-BRAF fusion nucleic acid molecule, in the 5′ to 3′ direction, comprising or resulting from a breakpoint within chromosomal coordinates chr7:140484314 and/or chr17:55727394. In some embodiments, a BRAF fusion nucleic acid molecule provided herein is a TNS3-BRAF fusion nucleic acid molecule, in the 5′ to 3′ direction, comprising or resulting from a breakpoint within chromosomal coordinates chr7:140483070 and/or chr7:47407649. In some embodiments, a BRAF fusion nucleic acid molecule provided herein is a BRAF-DOCK4 fusion nucleic acid molecule, in the 5′ to 3′ direction, comprising or resulting from a breakpoint within chromosomal coordinates chr7:140482165 and/or chr7:111379645. In some embodiments, a BRAF fusion nucleic acid molecule provided herein is a BRAF-RAD51 fusion nucleic acid molecule, in the 5′ to 3′ direction, comprising or resulting from a breakpoint within chromosomal coordinates chr15:41023127 and/or chr7:140501694. In some embodiments, a BRAF fusion nucleic acid molecule provided herein is an AKAP9-BRAF fusion nucleic acid molecule, in the 5′ to 3′ direction, comprising or resulting from a breakpoint within chromosomal coordinates chr7:140493457 and/or chr7:91701220. In some embodiments, a BRAF fusion nucleic acid molecule provided herein is an ARMC10-BRAF fusion nucleic acid molecule, in the 5′ to 3′ direction, comprising or resulting from a breakpoint within chromosomal coordinates chr7:140481643 and/or chr7:102730767. In some embodiments, a BRAF fusion nucleic acid molecule provided herein is a DENND2A-BRAF fusion nucleic acid molecule, in the 5′ to 3′ direction, comprising or resulting from a breakpoint within chromosomal coordinates chr7:140487304 and/or chr7:140243015. In some embodiments, a BRAF fusion nucleic acid molecule provided herein is a JHDM1D-BRAF fusion nucleic acid molecule, in the 5′ to 3′ direction, comprising or resulting from a breakpoint within chromosomal coordinates chr7:140488072 and/or chr7:139800753. In some embodiments, a BRAF fusion nucleic acid molecule provided herein is a KIAA1549-BRAF fusion nucleic acid molecule, in the 5′ to 3′ direction, comprising or resulting from a breakpoint within chromosomal coordinates chr7:140485311 and/or chr7:138564061. In some embodiments, a BRAF fusion nucleic acid molecule provided herein is an MKRN1-BRAF fusion nucleic acid molecule, in the 5′ to 3′ direction, comprising or resulting from a breakpoint within chromosomal coordinates chr7:140481605 and/or chr7:140157871. In some embodiments, a BRAF fusion nucleic acid molecule provided herein is a NRF1-BRAF fusion nucleic acid molecule, in the 5′ to 3′ direction, comprising or resulting from a breakpoint within chromosomal coordinates chr7:140485113 and/or chr7:129373037. In some embodiments, a BRAF fusion nucleic acid molecule provided herein is an SLC45A3-BRAF fusion nucleic acid molecule, in the 5′ to 3′ direction, comprising or resulting from a breakpoint within chromosomal coordinates chr7:140499628 and/or chr1:205639559. In some embodiments, a BRAF fusion nucleic acid molecule provided herein is an SND1-BRAF fusion nucleic acid molecule, in the 5′ to 3′ direction, comprising or resulting from a breakpoint within chromosomal coordinates chr7:140487680 and/or chr7:127715943. In some embodiments, a BRAF fusion nucleic acid molecule provided herein is an SND1-BRAF fusion nucleic acid molecule, in the 5′ to 3′ direction, comprising or resulting from a breakpoint within chromosomal coordinates chr7:140491295 and/or chr7:127399380. In some embodiments, a BRAF fusion nucleic acid molecule provided herein is an SND1-BRAF fusion nucleic acid molecule, in the 5′ to 3′ direction, comprising or resulting from a breakpoint within chromosomal coordinates chr7:140481458-140481757 and/or chr7:127356978-127357015. In some embodiments, a BRAF fusion nucleic acid molecule provided herein is an BRAF-TRIM24 fusion nucleic acid molecule, in the 5′ to 3′ direction, comprising or resulting from a breakpoint within chromosomal coordinates chr7:140490521 and/or chr7:138224175. In some embodiments, a BRAF fusion nucleic acid molecule provided herein is a ZC3HAV1-BRAF fusion nucleic acid molecule, in the 5′ to 3′ direction, comprising or resulting from a breakpoint within chromosomal coordinates chr7:140485942 and/or chr7:138765898. In some embodiments, a BRAF fusion nucleic acid molecule provided herein is a ZNF277-BRAF fusion nucleic acid molecule, in the 5′ to 3′ direction, comprising or resulting from a breakpoint within chromosomal coordinates chr7:140481495 and/or chr7:111929341. In some embodiments, a BRAF fusion nucleic acid molecule provided herein is a BRAF fusion nucleic acid molecule listed in any of Tables 3, 5, or 6, comprising or resulting from a Breakpoint 1 and/or Breakpoint 2 within the corresponding chromosomal coordinates as listed in any of Tables 3, 5, or 6.

In some aspects, provided herein are EGFR fusion nucleic acid molecules comprising at least a portion of EGFR and at least a portion of another gene. In some embodiments, an EGFR fusion nucleic acid molecule provided herein is an ABCB1-EGFR fusion nucleic acid molecule, in the 5′ to 3′ direction, comprising or resulting from a breakpoint within chromosomal coordinates chr7:55269027 and/or chr7:87226070. In some embodiments, an EGFR fusion nucleic acid molecule provided herein is a PDE7A-EGFR fusion nucleic acid molecule, in the 5′ to 3′ direction, comprising or resulting from a breakpoint within chromosomal coordinates chr7:55222346 and/or chr8:66656455. In some embodiments, an EGFR fusion nucleic acid molecule provided herein is an EGFR-EZH2 fusion nucleic acid molecule, in the 5′ to 3′ direction, comprising or resulting from a breakpoint within chromosomal coordinates chr7:55268176 and/or chr7:148565978. In some embodiments, an EGFR fusion nucleic acid molecule provided herein is an EGFR-FLJ45974 fusion nucleic acid molecule, in the 5′ to 3′ direction, comprising or resulting from a breakpoint within chromosomal coordinates chr7:55268138 and/or chr7:53830747. In some embodiments, an EGFR fusion nucleic acid molecule provided herein is an EGFR-ZNF479 fusion nucleic acid molecule, in the 5′ to 3′ direction, comprising or resulting from a breakpoint within chromosomal coordinates chr7:55269394 and/or chr7:57202770. In some embodiments, an EGFR fusion nucleic acid molecule provided herein is an EGFR fusion nucleic acid molecule listed in Table 3 or 5, comprising or resulting from a Breakpoint 1 and/or Breakpoint 2 within the corresponding chromosomal coordinates as listed in any of Tables 3 or 5.

In some aspects, provided herein are ERBB2 fusion nucleic acid molecules comprising at least a portion of ERBB2 and at least a portion of another gene. In some embodiments, an ERBB2 fusion nucleic acid molecule provided herein is an FBXL20-ERBB2 fusion nucleic acid molecule, in the 5′ to 3′ direction, comprising or resulting from a breakpoint within chromosomal coordinates chr17:37876145-37876247 and/or chr17:37472082-37472213. In some embodiments, an ERBB2 fusion nucleic acid molecule provided herein is a GRB7-ERBB2 fusion nucleic acid molecule, in the 5′ to 3′ direction, comprising or resulting from a breakpoint within chromosomal coordinates chr17:37881861-37882182 and/or chr17:37896410-37896567. In some embodiments, an ERBB2 fusion nucleic acid molecule provided herein is an MSI2-ERBB2 fusion nucleic acid molecule, in the 5′ to 3′ direction, comprising or resulting from a breakpoint within chromosomal coordinates chr17:37872528 and/or chr17:55725868. In some embodiments, an ERBB2 fusion nucleic acid molecule provided herein is a RANBP10-ERBB2 fusion nucleic acid molecule, in the 5′ to 3′ direction, comprising or resulting from a breakpoint within chromosomal coordinates chr17:37865475 and/or chr16:67790631. In some embodiments, an ERBB2 fusion nucleic acid molecule provided herein is an SEC14L1-ERBB2 fusion nucleic acid molecule, in the 5′ to 3′ direction, comprising or resulting from a breakpoint within chromosomal coordinates chr17:37879770 and/or chr17:75181466. In some embodiments, an ERBB2 fusion nucleic acid molecule provided herein is a WIPF2-ERBB2 fusion nucleic acid molecule, in the 5′ to 3′ direction, comprising or resulting from a breakpoint within chromosomal coordinates chr17:37881750 and/or chr17:38419094. In some embodiments, an ERBB2 fusion nucleic acid molecule provided herein is an ERBB2-GRB7 fusion nucleic acid molecule, in the 5′ to 3′ direction, comprising or resulting from a breakpoint within chromosomal coordinates chr17:37881373-37881485 and/or chr17:37902966-37903081. In some embodiments, an ERBB2 fusion nucleic acid molecule provided herein is an ERBB2-PRKCA fusion nucleic acid molecule, in the 5′ to 3′ direction, comprising or resulting from a breakpoint within chromosomal coordinates chr17:37872883 and/or chr17:64588788. In some embodiments, an ERBB2 fusion nucleic acid molecule provided herein is an ERBB2-PPP1R1B fusion nucleic acid molecule, in the 5′ to 3′ direction, comprising or resulting from a breakpoint within chromosomal coordinates chr17:37883936 and/or chr17:37789550. In some embodiments, an ERBB2 fusion nucleic acid molecule provided herein is an ERBB2 fusion nucleic acid molecule listed in any of Tables 3, 5, or 6, comprising or resulting from a Breakpoint 1 and/or Breakpoint 2 within the corresponding chromosomal coordinates as listed in any of Tables 3, 5, or 6.

In some aspects, provided herein are FGFR1 fusion nucleic acid molecules comprising at least a portion of FGFR1 and at least a portion of another gene. In some embodiments, an FGFR1 fusion nucleic acid molecule provided herein is an FGFR1-ADAM32 fusion nucleic acid molecule, in the 5′ to 3′ direction, comprising or resulting from a breakpoint within chromosomal coordinates chr8:38271185 and/or chr8:39031195. In some embodiments, an FGFR1 fusion nucleic acid molecule provided herein is an FGFR1-SLC12A8 fusion nucleic acid molecule, in the 5′ to 3′ direction, comprising or resulting from a breakpoint within chromosomal coordinates chr8:38285341 and/or chr3:124897236. In some embodiments, an FGFR1 fusion nucleic acid molecule provided herein is an ADAM18-FGFR1 fusion nucleic acid molecule, in the 5′ to 3′ direction, comprising or resulting from a breakpoint within chromosomal coordinates chr8:38271241 and/or chr8:39537661. In some embodiments, an FGFR1 fusion nucleic acid molecule provided herein is a BAG4-FGFR1 fusion nucleic acid molecule, in the 5′ to 3′ direction, comprising or resulting from a breakpoint within chromosomal coordinates chr8:38272085-38272258 and/or chr8:38036460-38036578. In some embodiments, an FGFR1 fusion nucleic acid molecule provided herein is an FGFR1-TACC1 fusion nucleic acid molecule, in the 5′ to 3′ direction, comprising or resulting from a breakpoint within chromosomal coordinates chr8:38273440 and/or chr8:38622825. In some embodiments, an FGFR1 fusion nucleic acid molecule provided herein is an FGFR1 fusion nucleic acid molecule listed in any of Tables 3, 5, or 6, comprising or resulting from a Breakpoint 1 and/or Breakpoint 2 within the corresponding chromosomal coordinates as listed in any of Tables 3, 5, or 6.

In some aspects, provided herein are FGFR2 fusion nucleic acid molecules comprising at least a portion of FGFR2 and at least a portion of another gene. In some embodiments, an FGFR2 fusion nucleic acid molecule provided herein is an FGFR2-AARSD1 fusion nucleic acid molecule, in the 5′ to 3′ direction, comprising or resulting from a breakpoint within chromosomal coordinates chr10:123240088 and/or chr17:41114482. In some embodiments, an FGFR2 fusion nucleic acid molecule provided herein is an FGFR2-ARMS2 fusion nucleic acid molecule, in the 5′ to 3′ direction, comprising or resulting from a breakpoint within chromosomal coordinates chr10:123240028 and/or chr10:124215085. In some embodiments, an FGFR2 fusion nucleic acid molecule provided herein is an FGFR2-ATF7 fusion nucleic acid molecule, in the 5′ to 3′ direction, comprising or resulting from a breakpoint within chromosomal coordinates chr10:123241999 and/or chr12:53951727. In some embodiments, an FGFR2 fusion nucleic acid molecule provided herein is an FGFR2-BAIAP2L1 fusion nucleic acid molecule, in the 5′ to 3′ direction, comprising or resulting from a breakpoint within chromosomal coordinates chr10:123243016-123243349 and/or chr7:98025564-98025703. In some embodiments, an FGFR2 fusion nucleic acid molecule provided herein is an FGFR2-CCAR1 fusion nucleic acid molecule, in the 5′ to 3′ direction, comprising or resulting from a breakpoint within chromosomal coordinates chr10:123240191 and/or chr10:70499696. In some embodiments, an FGFR2 fusion nucleic acid molecule provided herein is an FGFR2-CCSER2 fusion nucleic acid molecule, in the 5′ to 3′ direction, comprising or resulting from a breakpoint within chromosomal coordinates chr10:123241043 and/or chr10:86165140. In some embodiments, an FGFR2 fusion nucleic acid molecule provided herein is an FGFR2-CGNL1 fusion nucleic acid molecule, in the 5′ to 3′ direction, comprising or resulting from a breakpoint within chromosomal coordinates chr10:123242868 and/or chr15:57824138. In some embodiments, an FGFR2 fusion nucleic acid molecule provided herein is an FGFR2-EBF1 fusion nucleic acid molecule, in the 5′ to 3′ direction, comprising or resulting from a breakpoint within chromosomal coordinates chr10:123239923-123240116 and/or chr5:158518930-158519130. In some embodiments, an FGFR2 fusion nucleic acid molecule provided herein is an FGFR2-FANK1 fusion nucleic acid molecule, in the 5′ to 3′ direction, comprising or resulting from a breakpoint within chromosomal coordinates chr10:123241189 and/or chr10:127587833. In some embodiments, an FGFR2 fusion nucleic acid molecule provided herein is an FGFR2-FOXP1 fusion nucleic acid molecule, in the 5′ to 3′ direction, comprising or resulting from a breakpoint within chromosomal coordinates chr3:71189036-71189121 and/or chr10:123242151-123242274. In some embodiments, an FGFR2 fusion nucleic acid molecule provided herein is a CAMK2G-FGFR2 fusion nucleic acid molecule, in the 5′ to 3′ direction, comprising or resulting from a breakpoint within chromosomal coordinates chr10:123241878-123241987 and/or chr10:75603526-75603626. In some embodiments, an FGFR2 fusion nucleic acid molecule provided herein is an FLJ40288-FGFR2 fusion nucleic acid molecule, in the 5′ to 3′ direction, comprising or resulting from a breakpoint within chromosomal coordinates chr10:123242192 and/or chr7:132389880. In some embodiments, an FGFR2 fusion nucleic acid molecule provided herein is a GUCY2D-FGFR2 fusion nucleic acid molecule, in the 5′ to 3′ direction, comprising or resulting from a breakpoint within chromosomal coordinates chr10:123242050-123242262 and/or chr17:7910782-7911004. In some embodiments, an FGFR2 fusion nucleic acid molecule provided herein is an IQGAP2-FGFR2 fusion nucleic acid molecule, in the 5′ to 3′ direction, comprising or resulting from a breakpoint within chromosomal coordinates chr10:123241258 and/or chr5:75977315. In some embodiments, an FGFR2 fusion nucleic acid molecule provided herein is a PAWR-FGFR2 fusion nucleic acid molecule, in the 5′ to 3′ direction, comprising or resulting from a breakpoint within chromosomal coordinates chr10:123241205 and/or chr12:80080770. In some embodiments, an FGFR2 fusion nucleic acid molecule provided herein is an FGFR2-FLNB fusion nucleic acid molecule, in the 5′ to 3′ direction, comprising or resulting from a breakpoint within chromosomal coordinates chr10:123242707-123242915 and/or chr3:58121075-58121276. In some embodiments, an FGFR2 fusion nucleic acid molecule provided herein is an FGFR2-FLNB fusion nucleic acid molecule, in the 5′ to 3′ direction, comprising or resulting from a breakpoint within chromosomal coordinates chr10:123240801 and/or chr3:58143720. In some embodiments, an FGFR2 fusion nucleic acid molecule provided herein is an FGFR2-FOXP1 fusion nucleic acid molecule, in the 5′ to 3′ direction, comprising or resulting from a breakpoint within chromosomal coordinates chr10:123242151-123242274 and/or chr3:71189036-71189121. In some embodiments, an FGFR2 fusion nucleic acid molecule provided herein is an FGFR2-IKZF2 fusion nucleic acid molecule, in the 5′ to 3′ direction, comprising or resulting from a breakpoint within chromosomal coordinates chr10:123241884 and/or chr2:214016023. In some embodiments, an FGFR2 fusion nucleic acid molecule provided herein is an FGFR2-KHDRBS1 fusion nucleic acid molecule, in the 5′ to 3′ direction, comprising or resulting from a breakpoint within chromosomal coordinates chr10:123242626 and/or chr1:32497117. In some embodiments, an FGFR2 fusion nucleic acid molecule provided herein is an FGFR2-MYOZ1 fusion nucleic acid molecule, in the 5′ to 3′ direction, comprising or resulting from a breakpoint within chromosomal coordinates chr10:123242901 and/or chr10:75400338. In some embodiments, an FGFR2 fusion nucleic acid molecule provided herein is an FGFR2-PCDH15 fusion nucleic acid molecule, in the 5′ to 3′ direction, comprising or resulting from a breakpoint within chromosomal coordinates chr10:123257992-123258171 and/or chr10:55668437-55668600. In some embodiments, an FGFR2 fusion nucleic acid molecule provided herein is an FGFR2-PRKAR1A fusion nucleic acid molecule, in the 5′ to 3′ direction, comprising or resulting from a breakpoint within chromosomal coordinates chr10:123240937 and/or chr17:66496703. In some embodiments, an FGFR2 fusion nucleic acid molecule provided herein is an FGFR2-PRRC2A fusion nucleic acid molecule, in the 5′ to 3′ direction, comprising or resulting from a breakpoint within chromosomal coordinates chr10:123242694 and/or chr6:31595369. In some embodiments, an FGFR2 fusion nucleic acid molecule provided herein is an FGFR2-RABGAP1 fusion nucleic acid molecule, in the 5′ to 3′ direction, comprising or resulting from a breakpoint within chromosomal coordinates chr10:123241892 and/or chr9:125850348. In some embodiments, an FGFR2 fusion nucleic acid molecule provided herein is an FGFR2-SCIN fusion nucleic acid molecule, in the 5′ to 3′ direction, comprising or resulting from a breakpoint within chromosomal coordinates chr10:123239291 and/or chr7:12640299. In some embodiments, an FGFR2 fusion nucleic acid molecule provided herein is an FGFR2-STAU1 fusion nucleic acid molecule, in the 5′ to 3′ direction, comprising or resulting from a breakpoint within chromosomal coordinates chr10:123240764 and/or chr20:47762803. In some embodiments, an FGFR2 fusion nucleic acid molecule provided herein is an FGFR2-STK4 fusion nucleic acid molecule, in the 5′ to 3′ direction, comprising or resulting from a breakpoint within chromosomal coordinates chr10:123239784 and/or chr20:43702262. In some embodiments, an FGFR2 fusion nucleic acid molecule provided herein is an FGFR2-TIFA fusion nucleic acid molecule, in the 5′ to 3′ direction, comprising or resulting from a breakpoint within chromosomal coordinates chr10:123242222 and/or chr4:113200693. In some embodiments, an FGFR2 fusion nucleic acid molecule provided herein is an FGFR2-TLK1 fusion nucleic acid molecule, in the 5′ to 3′ direction, comprising or resulting from a breakpoint within chromosomal coordinates chr10:123239464 and/or chr2:171931100. In some embodiments, an FGFR2 fusion nucleic acid molecule provided herein is an FGFR2-TRIM54 fusion nucleic acid molecule, in the 5′ to 3′ direction, comprising or resulting from a breakpoint within chromosomal coordinates chr10:123240717 and/or chr2:27516917. In some embodiments, an FGFR2 fusion nucleic acid molecule provided herein is an FGFR2-APIP fusion nucleic acid molecule, in the 5′ to 3′ direction, comprising or resulting from a breakpoint within chromosomal coordinates chr10:123240839 and/or chr11:34921475. In some embodiments, an FGFR2 fusion nucleic acid molecule provided herein is an FGFR2-ATE1 fusion nucleic acid molecule, in the 5′ to 3′ direction, comprising or resulting from a breakpoint within chromosomal coordinates chr10:123241676 and/or chr10:123551234. In some embodiments, an FGFR2 fusion nucleic acid molecule provided herein is an FGFR2-BICC1 fusion nucleic acid molecule, in the 5′ to 3′ direction, comprising or resulting from a breakpoint within chromosomal coordinates chr10:123241383 and/or chr10:60410103. In some embodiments, an FGFR2 fusion nucleic acid molecule provided herein is an FGFR2-BICC1 fusion nucleic acid molecule, in the 5′ to 3′ direction, comprising or resulting from a breakpoint within chromosomal coordinates chr10:123241708 and/or chr10:60428801. In some embodiments, an FGFR2 fusion nucleic acid molecule provided herein is an FGFR2-BICC1 fusion nucleic acid molecule, in the 5′ to 3′ direction, comprising or resulting from a breakpoint within chromosomal coordinates chr10:123239702 and/or chr10:60429695. In some embodiments, an FGFR2 fusion nucleic acid molecule provided herein is a TFEC-FGFR2 fusion nucleic acid molecule, in the 5′ to 3′ direction, comprising or resulting from a breakpoint within chromosomal coordinates chr10:123240329 and/or chr7:115606014. In some embodiments, an FGFR2 fusion nucleic acid molecule provided herein is an FGFR2-GRB2 fusion nucleic acid molecule, in the 5′ to 3′ direction, comprising or resulting from a breakpoint within chromosomal coordinates chr10:123241875 and/or chr17:73384322. In some embodiments, an FGFR2 fusion nucleic acid molecule provided herein is an FGFR2-GRB2 fusion nucleic acid molecule, in the 5′ to 3′ direction, comprising or resulting from a breakpoint within chromosomal coordinates chr10:123242159 and/or chr17:73365008. In some embodiments, an FGFR2 fusion nucleic acid molecule provided herein is an FGFR2-KIAA1217 fusion nucleic acid molecule, in the 5′ to 3′ direction, comprising or resulting from a breakpoint within chromosomal coordinates chr10:123241180 and/or chr10:24605241. In some embodiments, an FGFR2 fusion nucleic acid molecule provided herein is an FGFR2-KIAA1598 fusion nucleic acid molecule, in the 5′ to 3′ direction, comprising or resulting from a breakpoint within chromosomal coordinates chr10:123242758-123242929 and/or chr10:118708900-118709045. In some embodiments, an FGFR2 fusion nucleic acid molecule provided herein is an FGFR2-MACF1 fusion nucleic acid molecule, in the 5′ to 3′ direction, comprising or resulting from a breakpoint within chromosomal coordinates chr10:123241285 and/or chr1:39918104. In some embodiments, an FGFR2 fusion nucleic acid molecule provided herein is an FGFR2-MYH9 fusion nucleic acid molecule, in the 5′ to 3′ direction, comprising or resulting from a breakpoint within chromosomal coordinates chr10:123240474-123240649 and/or chr22:36695796-36695923. In some embodiments, an FGFR2 fusion nucleic acid molecule provided herein is an FGFR2-NRAP fusion nucleic acid molecule, in the 5′ to 3′ direction, comprising or resulting from a breakpoint within chromosomal coordinates chr10:123241635 and/or chr10:115380780. In some embodiments, an FGFR2 fusion nucleic acid molecule provided herein is an FGFR2-RBM20 fusion nucleic acid molecule, in the 5′ to 3′ direction, comprising or resulting from a breakpoint within chromosomal coordinates chr10:123241293 and/or chr10:112580773. In some embodiments, an FGFR2 fusion nucleic acid molecule provided herein is an FGFR2-SPICE1 fusion nucleic acid molecule, in the 5′ to 3′ direction, comprising or resulting from a breakpoint within chromosomal coordinates chr10:123239840-123239995 and/or chr3:113207848-113207958. In some embodiments, an FGFR2 fusion nucleic acid molecule provided herein is an FGFR2-TACC2 fusion nucleic acid molecule, in the 5′ to 3′ direction, comprising or resulting from a breakpoint within chromosomal coordinates chr10:123239675-123239796 and/or chr10:123988441-123988546. In some embodiments, an FGFR2 fusion nucleic acid molecule provided herein is an FGFR2-TACC2 fusion nucleic acid molecule, in the 5′ to 3′ direction, comprising or resulting from a breakpoint within chromosomal coordinates chr10:123241654-123241788 and/or chr10:123989561-123989655. In some embodiments, an FGFR2 fusion nucleic acid molecule provided herein is an FGFR2-TACC2 fusion nucleic acid molecule, in the 5′ to 3′ direction, comprising or resulting from a breakpoint within chromosomal coordinates chr10:123239631 and/or chr10:123988734. In some embodiments, an FGFR2 fusion nucleic acid molecule provided herein is an FGFR2-TACC2 fusion nucleic acid molecule, in the 5′ to 3′ direction, comprising or resulting from a breakpoint within chromosomal coordinates chr10:123241742 and/or chr10:123987953. In some embodiments, an FGFR2 fusion nucleic acid molecule provided herein is an FGFR2-VTI1A fusion nucleic acid molecule, in the 5′ to 3′ direction, comprising or resulting from a breakpoint within chromosomal coordinates chr10:123239845 and/or chr10:114543839. In some embodiments, an FGFR2 fusion nucleic acid molecule provided herein is an FGFR2-WAC fusion nucleic acid molecule, in the 5′ to 3′ direction, comprising or resulting from a breakpoint within chromosomal coordinates chr10:123242151-123242331 and/or chr10:28905673-28905852. In some embodiments, an FGFR2 fusion nucleic acid molecule provided herein is an FGFR2-WARS fusion nucleic acid molecule, in the 5′ to 3′ direction, comprising or resulting from a breakpoint within chromosomal coordinates chr10:123239968 and/or chr14:100834590. In some embodiments, an FGFR2 fusion nucleic acid molecule provided herein is an FGFR2-WARS fusion nucleic acid molecule, in the 5′ to 3′ direction, comprising or resulting from a breakpoint within chromosomal coordinates chr10:123242027-123242147 and/or chr14:100829090-100829181. In some embodiments, an FGFR2 fusion nucleic acid molecule provided herein is an FGFR2-ZMYM4 fusion nucleic acid molecule, in the 5′ to 3′ direction, comprising or resulting from a breakpoint within chromosomal coordinates chr10:123241846 and/or chr1:35851725. In some embodiments, an FGFR2 fusion nucleic acid molecule provided herein is an FGFR2 fusion nucleic acid molecule listed in any of Tables 3, 5, or 6, comprising or resulting from a Breakpoint 1 and/or Breakpoint 2 within the corresponding chromosomal coordinates as listed in any of Tables 3, 5, or 6.

In some aspects, provided herein are FGFR3 fusion nucleic acid molecules comprising at least a portion of FGFR3 and at least a portion of another gene. In some embodiments, an FGFR3 fusion nucleic acid molecule provided herein is an FGFR3-ADD1 fusion nucleic acid molecule, in the 5′ to 3′ direction, comprising or resulting from a breakpoint within chromosomal coordinates chr4:1808801 and/or chr4:2882826. In some embodiments, an FGFR3 fusion nucleic acid molecule provided herein is an FGFR3-CCT5 fusion nucleic acid molecule, in the 5′ to 3′ direction, comprising or resulting from a breakpoint within chromosomal coordinates chr4:1808575-1808796 and/or chr5:10252744-10252900. In some embodiments, an FGFR3 fusion nucleic acid molecule provided herein is an FGFR3-CNOT4 fusion nucleic acid molecule, in the 5′ to 3′ direction, comprising or resulting from a breakpoint within chromosomal coordinates chr4:1805827-1805962 and/or chr7:135079969-135080300. In some embodiments, an FGFR3 fusion nucleic acid molecule provided herein is an FGFR3-TNIP2 fusion nucleic acid molecule, in the 5′ to 3′ direction, comprising or resulting from a breakpoint within chromosomal coordinates chr4:1808845 and/or chr4:2754775. In some embodiments, an FGFR3 fusion nucleic acid molecule provided herein is an FGFR3-IGH fusion nucleic acid molecule, in the 5′ to 3′ direction, comprising or resulting from a breakpoint within chromosomal coordinates chr4:1808551 and/or chr14:106005444. In some embodiments, an FGFR3 fusion nucleic acid molecule provided herein is an FGFR3-TACC3 fusion nucleic acid molecule, in the 5′ to 3′ direction, comprising or resulting from a breakpoint within chromosomal coordinates chr4:1808563-1808711 and/or chr4:1730156-1730288. In some embodiments, an FGFR3 fusion nucleic acid molecule provided herein is an FGFR3-TACC3 fusion nucleic acid molecule, in the 5′ to 3′ direction, comprising or resulting from a breakpoint within chromosomal coordinates chr4:1808845 and/or chr4:1739165. In some embodiments, an FGFR3 fusion nucleic acid molecule provided herein is an FGFR3-TACC3 fusion nucleic acid molecule, in the 5′ to 3′ direction, comprising or resulting from a breakpoint within chromosomal coordinates chr4:1808751 and/or chr4:1739667. In some embodiments, an FGFR3 fusion nucleic acid molecule provided herein is an FGFR3-TACC3 fusion nucleic acid molecule, in the 5′ to 3′ direction, comprising or resulting from a breakpoint within chromosomal coordinates chr4:1808710 and/or chr4:1740755. In some embodiments, an FGFR3 fusion nucleic acid molecule provided herein is an FGFR3-TACC3 fusion nucleic acid molecule, in the 5′ to 3′ direction, comprising or resulting from a breakpoint within chromosomal coordinates chr4:1808729 and/or chr4:1739043. In some embodiments, an FGFR3 fusion nucleic acid molecule provided herein is an FGFR3-TACC3 fusion nucleic acid molecule, in the 5′ to 3′ direction, comprising or resulting from a breakpoint within chromosomal coordinates chr4:1808705 and/or chr4:1739261. In some embodiments, an FGFR3 fusion nucleic acid molecule provided herein is an FGFR3-TACC3 fusion nucleic acid molecule, in the 5′ to 3′ direction, comprising or resulting from a breakpoint within chromosomal coordinates chr4:1808678 and/or chr4:1738998. In some embodiments, an FGFR3 fusion nucleic acid molecule provided herein is an FGFR3-TACC3 fusion nucleic acid molecule, in the 5′ to 3′ direction, comprising or resulting from a breakpoint within chromosomal coordinates chr4:1808911 and/or chr4:1739886. In some embodiments, an FGFR3 fusion nucleic acid molecule provided herein is an FGFR3-TACC3 fusion nucleic acid molecule, in the 5′ to 3′ direction, comprising or resulting from a breakpoint within chromosomal coordinates chr4:1808671 and/or chr4:1741144. In some embodiments, an FGFR3 fusion nucleic acid molecule provided herein is an FGFR3-WHSC1 fusion nucleic acid molecule, in the 5′ to 3′ direction, comprising or resulting from a breakpoint within chromosomal coordinates chr4:1808442-1808709 and/or chr4:1949176-1949445. In some embodiments, an FGFR3 fusion nucleic acid molecule provided herein is an FGFR3 fusion nucleic acid molecule listed in any of Tables 3, 5, or 6, comprising or resulting from a Breakpoint 1 and/or Breakpoint 2 within the corresponding chromosomal coordinates as listed in any of Tables 3, 5, or 6.

In some aspects, provided herein are MET fusion nucleic acid molecules comprising at least a portion of MET and at least a portion of another gene. In some embodiments, a MET fusion nucleic acid molecule provided herein is a MET-LDHA fusion nucleic acid molecule, in the 5′ to 3′ direction, comprising or resulting from a breakpoint within chromosomal coordinates chr7:116411832-116411915 and/or chr11:18429175-18429254. In some embodiments, a MET fusion nucleic acid molecule provided herein is a CNTNAP2-MET fusion nucleic acid molecule, in the 5′ to 3′ direction, comprising or resulting from a breakpoint within chromosomal coordinates chr7:116398519 and/or chr7:145963989. In some embodiments, a MET fusion nucleic acid molecule provided herein is an HBP1-MET fusion nucleic acid molecule, in the 5′ to 3′ direction, comprising or resulting from a breakpoint within chromosomal coordinates chr7:116339469-116339610 and/or chr7:106814702-106814833. In some embodiments, a MET fusion nucleic acid molecule provided herein is an SNRNP70-MET fusion nucleic acid molecule, in the 5′ to 3′ direction, comprising or resulting from a breakpoint within chromosomal coordinates chr7:116412044-116412245 and/or chr19:49598459-49598574. In some embodiments, a MET fusion nucleic acid molecule provided herein is an MET-CAPZA2 fusion nucleic acid molecule, in the 5′ to 3′ direction, comprising or resulting from a breakpoint within chromosomal coordinates chr7:116435955 and/or chr7:116506109. In some embodiments, a MET fusion nucleic acid molecule provided herein is a ST7-MET fusion nucleic acid molecule, in the 5′ to 3′ direction, comprising or resulting from a breakpoint within chromosomal coordinates chr7:116399431 and/or chr7:116726687. In some embodiments, a MET fusion nucleic acid molecule provided herein is a ST7-MET fusion nucleic acid molecule, in the 5′ to 3′ direction, comprising or resulting from a breakpoint within chromosomal coordinates chr7:116399373 and/or chr7:116803090. In some embodiments, a MET fusion nucleic acid molecule provided herein is a MET fusion nucleic acid molecule listed in any of Tables 3, 5, or 6, comprising or resulting from a Breakpoint 1 and/or Breakpoint 2 within the corresponding chromosomal coordinates as listed in any of Tables 3, 5, or 6.

In some aspects, provided herein are NTRK1 fusion nucleic acid molecules comprising at least a portion of NTRK1 and at least a portion of another gene. In some embodiments, an NTRK1 fusion nucleic acid molecule provided herein is an NTRK1-MEF2D fusion nucleic acid molecule, in the 5′ to 3′ direction, comprising or resulting from a breakpoint within chromosomal coordinates chr1:156843853 and/or chr1:156441602. In some embodiments, an NTRK1 fusion nucleic acid molecule provided herein is an NTRK1 fusion nucleic acid molecule listed in Table 6, comprising or resulting from a Breakpoint 1 and/or Breakpoint 2 within the corresponding chromosomal coordinates as listed in Table 6.

In some aspects, provided herein are RAF1 fusion nucleic acid molecules comprising at least a portion of RAF1 and at least a portion of another gene. In some embodiments, a RAF1 fusion nucleic acid molecule provided herein is a POC1A-RAF1 fusion nucleic acid molecule, in the 5′ to 3′ direction, comprising or resulting from a breakpoint within chromosomal coordinates chr3:12642793 and/or chr3:52175928. In some embodiments, a RAF1 fusion nucleic acid molecule provided herein is a SYN2-RAF1 fusion nucleic acid molecule, in the 5′ to 3′ direction, comprising or resulting from a breakpoint within chromosomal coordinates chr3:12645756 and/or chr3:12210717. In some embodiments, a RAF1 fusion nucleic acid molecule provided herein is a ZFYVE20-RAF1 fusion nucleic acid molecule, in the 5′ to 3′ direction, comprising or resulting from a breakpoint within chromosomal coordinates chr3:12641467 and/or chr3:15112103. In some embodiments, a RAF1 fusion nucleic acid molecule provided herein is a RAF1-TRAK1 fusion nucleic acid molecule, in the 5′ to 3′ direction, comprising or resulting from a breakpoint within chromosomal coordinates chr3:12645483 and/or chr3:42231967. In some embodiments, a RAF1 fusion nucleic acid molecule provided herein is a RAF1 fusion nucleic acid molecule listed in any of Tables 3, 5, or 6, comprising or resulting from a Breakpoint 1 and/or Breakpoint 2 within the corresponding chromosomal coordinates as listed in any of Tables 3, 5, or 6.

In some aspects, provided herein are RET fusion nucleic acid molecule comprising at least a portion of RET and at least a portion of another gene. In some embodiments, a RET fusion nucleic acid molecule provided herein is an RET-ADCY1 fusion nucleic acid molecule, in the 5′ to 3′ direction, comprising or resulting from a breakpoint within chromosomal coordinates chr10:43610900-43611038 and/or chr7:45724501-45724758. In some embodiments, a RET fusion nucleic acid molecule provided herein is an BAIAP2L1-RET fusion nucleic acid molecule, in the 5′ to 3′ direction, comprising or resulting from a breakpoint within chromosomal coordinates chr10:43610040-43610258 and/or chr7:97943769-97943957. In some embodiments, a RET fusion nucleic acid molecule provided herein is an RET-NPY4R fusion nucleic acid molecule, in the 5′ to 3′ direction, comprising or resulting from a breakpoint within chromosomal coordinates chr10:43611694 and/or chr10:47084409. In some embodiments, a RET fusion nucleic acid molecule provided herein is an RET-PAWR fusion nucleic acid molecule, in the 5′ to 3′ direction, comprising or resulting from a breakpoint within chromosomal coordinates chr10:43610049-43610142 and/or chr12:80001152-80001299. In some embodiments, a RET fusion nucleic acid molecule provided herein is an ALOX5-RET fusion nucleic acid molecule, in the 5′ to 3′ direction, comprising or resulting from a breakpoint within chromosomal coordinates chr10:43610115 and/or chr10:45884309. In some embodiments, a RET fusion nucleic acid molecule provided herein is an ARID5B-RET fusion nucleic acid molecule, in the 5′ to 3′ direction, comprising or resulting from a breakpoint within chromosomal coordinates chr10:43610699 and/or chr10:63843922. In some embodiments, a RET fusion nucleic acid molecule provided herein is a DHX32-RET fusion nucleic acid molecule, in the 5′ to 3′ direction, comprising or resulting from a breakpoint within chromosomal coordinates chr10:43610814-43610918 and/or chr10:127554102-127554221. In some embodiments, a RET fusion nucleic acid molecule provided herein is a PDE5A-RET fusion nucleic acid molecule, in the 5′ to 3′ direction, comprising or resulting from a breakpoint within chromosomal coordinates chr10:43611716 and/or chr4:120458646. In some embodiments, a RET fusion nucleic acid molecule provided herein is a ZNF365-RET fusion nucleic acid molecule, in the 5′ to 3′ direction, comprising or resulting from a breakpoint within chromosomal coordinates chr10:43610404 and/or chr10:64337727. In some embodiments, a RET fusion nucleic acid molecule provided herein is a RET-CSGALNACT2 fusion nucleic acid molecule, in the 5′ to 3′ direction, comprising or resulting from a breakpoint within chromosomal coordinates chr10:43609615-43609862 and/or chr10:43651937-43652250. In some embodiments, a RET fusion nucleic acid molecule provided herein is a RET-GPHN fusion nucleic acid molecule, in the 5′ to 3′ direction, comprising or resulting from a breakpoint within chromosomal coordinates chr10:43611481 and/or chr14:67585496. In some embodiments, a RET fusion nucleic acid molecule provided herein is an NCOA4-RET fusion nucleic acid molecule, in the 5′ to 3′ direction, comprising or resulting from a breakpoint within chromosomal coordinates chr10:43611605-43611729 and/or chr10:51586055-51586121. In some embodiments, a RET fusion nucleic acid molecule provided herein is an NCOA4-RET fusion nucleic acid molecule, in the 5′ to 3′ direction, comprising or resulting from a breakpoint within chromosomal coordinates chr10:43611515 and/or chr10:51588839. In some embodiments, a RET fusion nucleic acid molecule provided herein is a RET-RASGEF1A fusion nucleic acid molecule, in the 5′ to 3′ direction, comprising or resulting from a breakpoint within chromosomal coordinates chr10:43597874 and/or chr10:43705242. In some embodiments, a RET fusion nucleic acid molecule provided herein is a KIAA1217-RET fusion nucleic acid molecule, in the 5′ to 3′ direction, comprising or resulting from a breakpoint within chromosomal coordinates chr10:43610121 and/or chr10:24816841. In some embodiments, a RET fusion nucleic acid molecule provided herein is a CCDC6-RET fusion nucleic acid molecule, in the 5′ to 3′ direction, comprising or resulting from a breakpoint within chromosomal coordinates chr10:43610721 and/or chr10:61653627. In some embodiments, a RET fusion nucleic acid molecule provided herein is a CCDC6-RET fusion nucleic acid molecule, in the 5′ to 3′ direction, comprising or resulting from a breakpoint within chromosomal coordinates chr10:43610931 and/or chr10:61639079. In some embodiments, a RET fusion nucleic acid molecule provided herein is an ERC1-RET fusion nucleic acid molecule, in the 5′ to 3′ direction, comprising or resulting from a breakpoint within chromosomal coordinates chr10:43610879 and/or chr12:1346557. In some embodiments, a RET fusion nucleic acid molecule provided herein is a KIAA1217-RET fusion nucleic acid molecule, in the 5′ to 3′ direction, comprising or resulting from a breakpoint within chromosomal coordinates chr10:43610513-43610815 and/or chr10:24813557-24813833. In some embodiments, a RET fusion nucleic acid molecule provided herein is a KIAA1217-RET fusion nucleic acid molecule, in the 5′ to 3′ direction, comprising or resulting from a breakpoint within chromosomal coordinates chr10:43611407 and/or chr10:24816256. In some embodiments, a RET fusion nucleic acid molecule provided herein is a KIFSB-RET fusion nucleic acid molecule, in the 5′ to 3′ direction, comprising or resulting from a breakpoint within chromosomal coordinates chr10:43610514-43610659 and/or chr10:32313549-32313707. In some embodiments, a RET fusion nucleic acid molecule provided herein is a KIFSB-RET fusion nucleic acid molecule, in the 5′ to 3′ direction, comprising or resulting from a breakpoint within chromosomal coordinates chr10:43609418 and/or chr10:32304634. In some embodiments, a RET fusion nucleic acid molecule provided herein is a KIFSB-RET fusion nucleic acid molecule, in the 5′ to 3′ direction, comprising or resulting from a breakpoint within chromosomal coordinates chr10:43611132-43611473 and/or chr10:32312409-32312729. In some embodiments, a RET fusion nucleic acid molecule provided herein is a TRIM24-RET fusion nucleic acid molecule, in the 5′ to 3′ direction, comprising or resulting from a breakpoint within chromosomal coordinates chr10:43611376-43611543 and/or chr7:138250812-138251052. In some embodiments, a RET fusion nucleic acid molecule provided herein is a VCL-RET fusion nucleic acid molecule, in the 5′ to 3′ direction, comprising or resulting from a breakpoint within chromosomal coordinates chr10:43611112-43611316 and/or chr10:75861515-75861716. In some embodiments, a RET fusion nucleic acid molecule provided herein is a RET fusion nucleic acid molecule listed in any of Tables 3, 5, or 6, comprising or resulting from a Breakpoint 1 and/or Breakpoint 2 within the corresponding chromosomal coordinates as listed in any of Tables 3, 5, or 6.

In some aspects, provided herein are ROS1 fusion nucleic acid molecules comprising at least a portion of ROS1 and at least a portion of another gene. In some embodiments, a ROS1 fusion nucleic acid molecule provided herein is a ROS1-ABR fusion nucleic acid molecule, in the 5′ to 3′ direction, comprising or resulting from a breakpoint within chromosomal coordinates chr6:117650357-117650571 and/or chr17:939714-939902. In some embodiments, a ROS1 fusion nucleic acid molecule provided herein is a ROS1-ASCC3 fusion nucleic acid molecule, in the 5′ to 3′ direction, comprising or resulting from a breakpoint within chromosomal coordinates chr6:117648934 and/or chr6:101139960. In some embodiments, a ROS1 fusion nucleic acid molecule provided herein is a ROS1-ELOVL4 fusion nucleic acid molecule, in the 5′ to 3′ direction, comprising or resulting from a breakpoint within chromosomal coordinates chr6:117647991 and/or chr6:80634367. In some embodiments, a ROS1 fusion nucleic acid molecule provided herein is a ROS1-QKI fusion nucleic acid molecule, in the 5′ to 3′ direction, comprising or resulting from a breakpoint within chromosomal coordinates chr6:117646322 and/or chr6:163894840. In some embodiments, a ROS1 fusion nucleic acid molecule provided herein is a ROS1-REV3L fusion nucleic acid molecule, in the 5′ to 3′ direction, comprising or resulting from a breakpoint within chromosomal coordinates chr6:117647122 and/or chr6:111770010. In some embodiments, a ROS1 fusion nucleic acid molecule provided herein is a MED23-ROS1 fusion nucleic acid molecule, in the 5′ to 3′ direction, comprising or resulting from a breakpoint within chromosomal coordinates chr6:117644495 and/or chr6:131914175. In some embodiments, a ROS1 fusion nucleic acid molecule provided herein is a SLC30A8-ROS1 fusion nucleic acid molecule, in the 5′ to 3′ direction, comprising or resulting from a breakpoint within chromosomal coordinates chr6:117645825 and/or chr8:118126691. In some embodiments, a ROS1 fusion nucleic acid molecule provided herein is an SLC38A11-ROS1 fusion nucleic acid molecule, in the 5′ to 3′ direction, comprising or resulting from a breakpoint within chromosomal coordinates chr6:117644675 and/or chr2:165777489. In some embodiments, a ROS1 fusion nucleic acid molecule provided herein is a TLN1-ROS1 fusion nucleic acid molecule, in the 5′ to 3′ direction, comprising or resulting from a breakpoint within chromosomal coordinates chr6:117642549 and/or chr9:35698416. In some embodiments, a ROS1 fusion nucleic acid molecule provided herein is a ROS1-SLC26A2 fusion nucleic acid molecule, in the 5′ to 3′ direction, comprising or resulting from a breakpoint within chromosomal coordinates chr6:117646090 and/or chr5:149340716. In some embodiments, a ROS1 fusion nucleic acid molecule provided herein is a ROS1-SYNGR1 fusion nucleic acid molecule, in the 5′ to 3′ direction, comprising or resulting from a breakpoint within chromosomal coordinates chr6:117649561 and/or chr22:39776088. In some embodiments, a ROS1 fusion nucleic acid molecule provided herein is a ROS1-TRPC6 fusion nucleic acid molecule, in the 5′ to 3′ direction, comprising or resulting from a breakpoint within chromosomal coordinates chr6:117647981 and/or chr11:101444124. In some embodiments, a ROS1 fusion nucleic acid molecule provided herein is an EZR-ROS1 fusion nucleic acid molecule, in the 5′ to 3′ direction, comprising or resulting from a breakpoint within chromosomal coordinates chr6:117645920-117646133 and/or chr6:159191019-159191124. In some embodiments, a ROS1 fusion nucleic acid molecule provided herein is a GOPC-ROS1 fusion nucleic acid molecule, in the 5′ to 3′ direction, comprising or resulting from a breakpoint within chromosomal coordinates chr6:117643144 and/or chr6:117886533. In some embodiments, a ROS1 fusion nucleic acid molecule provided herein is a GOPC-ROS1 fusion nucleic acid molecule, in the 5′ to 3′ direction, comprising or resulting from a breakpoint within chromosomal coordinates chr6:117643807 and/or chr6:117885081. In some embodiments, a ROS1 fusion nucleic acid molecule provided herein is a GOPC-ROS1 fusion nucleic acid molecule, in the 5′ to 3′ direction, comprising or resulting from a breakpoint within chromosomal coordinates chr6:117641972 and/or chr6:117894977. In some embodiments, a ROS1 fusion nucleic acid molecule provided herein is a GOPC-ROS1 fusion nucleic acid molecule, in the 5′ to 3′ direction, comprising or resulting from a breakpoint within chromosomal coordinates chr6:117645121 and/or chr6:117885917. In some embodiments, a ROS1 fusion nucleic acid molecule provided herein is a MYO5C-ROS1 fusion nucleic acid molecule, in the 5′ to 3′ direction, comprising or resulting from a breakpoint within chromosomal coordinates chr6:117642709-117642959 and/or chr15:52512166-52512328. In some embodiments, a ROS1 fusion nucleic acid molecule provided herein is a ROS1-TPD52L1 fusion nucleic acid molecule, in the 5′ to 3′ direction, comprising or resulting from a breakpoint within chromosomal coordinates chr6:117647493 and/or chr6:125568358. In some embodiments, a ROS1 fusion nucleic acid molecule provided herein is a ROS1 fusion nucleic acid molecule listed in any of Tables 3, 5, or 6, comprising or resulting from a Breakpoint 1 and/or Breakpoint 2 within the corresponding chromosomal coordinates as listed in any of Tables 3, 5, or 6.

TABLE 3
Exemplary ALK, BRAF, EGFR, ERBB2, FGFR1, FGFR2,
FGFR3, MET, RAF1, RET, and ROS1 fusion nucleic
acid molecules and the corresponding breakpoints.

| ALK | | | | |
|---|---|---|---|---|
| Fusion | | | | |
| Nucleic | | | | |
| Acid | | | | |
| Molecule | Breakpoint 1 | Breakpoint 2 | 5′ Gene | 3′ Gene |
| ALK- | chr2:29446320 | chr2:236984410 | AGAP1 | ALK |
| AGAP1 | | | | |
| ALK- | chr2:29448192 | chr13:111796690 | ARHGEF7 | ALK |
| ARHGEF7 | | | | |
| ALK-BRE | chr2:29448094- | chr2:28431656- | BRE | ALK |
| | 29448239 | 28431790 | | |
| ALK- | chr2:29448033 | chr12:15811779 | EPS8 | ALK |
| EPS8 | | | | |
| ALK- | chr2:29448344 | chr2:26546664 | GPR113 | ALK |
| GPR113 | | | | |
| ALK- | chr2:29420593 | chr7:18908783 | HDAC9 | ALK |
| HDAC9 | | | | |
| ALK- | chr2:29446669 | chr14:37906366 | MIPOL1 | ALK |
| MIPOL1 | | | | |
| ALK- | chr2:29448116 | chr2:64360629 | PELI1 | ALK |
| PELI1 | | | | |
| ALK- | chr2:29448047 | chr2:196583393 | SLC39A10 | ALK |
| SLC39A10 | | | | |
| ALK- | chr2:29447360- | chr7:65386539- | VKORCIL1 | ALK |
| VKORCIL1 | 29447477 | 65386741 | | |
| ALK- | chr2:29447840 | chr10:97287128 | ALK | SORBS1 |
| SORBS1 | | | | |
| ALK- | chr2:29448698 | chr5:147513014 | ALK | SPINK5 |
| SPINK5 | | | | |
| BRAF | | | | |
| Fusion | | | | |
| Nucleic | | | | |
| Acid | | | | |
| Molecule | Breakpoint 1 | Breakpoint 2 | 5′ Gene | 3′ Gene |
| BRAF- | chr7:140489237 | chr14:91742455 | CCDC88C | BRAF |
| CCDC88C | | | | |
| BRAF- | chr7:140489195 | chr2:165542493 | COBLL1 | BRAF |
| COBLL1 | | | | |
| BRAF- | chr7:140482515 | chr7:137655487 | CREB3L2 | BRAF |
| CREB3L2 | | | | |
| BRAF- | chr7:140434573 | chr8:13242758 | DLC1 | BRAF |
| DLC1 | | | | |
| BRAF- | chr7:140494157 | chr12:133360988 | GOLGA3 | BRAF |
| GOLGA3 | | | | |
| BRAF- | chr7:140484314 | chr17:55727394 | MSI2 | BRAF |
| MSI2 | | | | |
| BRAF- | chr7:140483070 | chr7:47407649 | TNS3 | BRAF |
| TNS3 | | | | |
| BRAF- | chr7:140482165 | chr7:111379645 | BRAF | DOCK4 |
| DOCK4 | | | | |
| BRAF- | chr15:41023127 | chr7:140501694 | BRAF | RAD51 |
| RAD51 | | | | |
| EGFR | | | | |
| Fusion | | | | |
| Nucleic | | | | |
| Acid | | | | |
| Molecule | Breakpoint 1 | Breakpoint 2 | 5′ Gene | 3′ Gene |
| EGFR- | chr7:55269027 | chr7:87226070 | ABCB1 | EGFR |
| ABCB1 | | | | |
| EGFR | chr7:55222346 | chr8:66656455 | PDE7A | EGFR |
| PDE7A | | | | |
| EGFR | chr7:55268176 | chr7:148565978 | EGFR | EZH2 |
| EZH2 | | | | |
| EGFR | chr7:55268138 | chr7:53830747 | EGFR | FLJ45974 |
| FLJ45974 | | | | |
| EGFR- | chr7:55269394 | chr7:57202770 | EGFR | ZNF479 |
| ZNF479 | | | | |
| ERBB2 | | | | |
| Fusion | | | | |
| Nucleic | | | | |
| Acid | | | | |
| Molecule | Breakpoint 1 | Breakpoint 2 | 5′ Gene | 3′ Gene |
| ERBB2- | chr17:37876145- | chr17:37472082- | FBXL20 | ERBB2 |
| FBXL20 | 37876247 | 37472213 | | |
| ERBB2- | chr17:37881861- | chr17:37896410- | GRB7 | ERBB2 |
| GRB7 | 37882182 | 37896567 | | |
| ERBB2- | chr17:37872528 | chr17:55725868 | MSI2 | ERBB2 |
| MSI2 | | | | |
| ERBB2- | chr17:37865475 | chr16:67790631 | RANBP10 | ERBB2 |
| RANBP10 | | | | |
| ERBB2- | chr17:37879770 | chr17:75181466 | SEC14L1 | ERBB2 |
| SEC14L1 | | | | |
| ERBB2- | chr17:37881750 | chr17:38419094 | WIPF2 | ERBB2 |
| WIPF2 | | | | |
| ERBB2- | chr17:37881373- | chr17:37902966- | ERBB2 | GRB7 |
| GRB7 | 37881485 | 37903081 | | |
| ERBB2- | chr17:37872883 | chr17:64588788 | ERBB2 | PRKCA |
| PRKCA | | | | |
| FGFR1 | | | | |
| Fusion | | | | |
| Nucleic | | | | |
| Acid | | | | |
| Molecule | Breakpoint 1 | Breakpoint 2 | 5′ Gene | 3′ Gene |
| FGFR1- | chr8:38271185 | chr8:39031195 | FGFR1 | ADAM32 |
| ADAM32 | | | | |
| FGFR1- | chr8:38285341 | chr3:124897236 | FGFR1 | SLC12A8 |
| SLC12A8 | | | | |
| FGFR2 | | | | |
| Fusion | | | | |
| Nucleic | | | | |
| Acid | | | | |
| Molecule | Breakpoint 1 | Breakpoint 2 | 5′ Gene | 3′ Gene |
| FGFR2- | chr10:123240088 | chr17:41114482 | FGFR2 | AARSD1 |
| AARSD1 | | | | |
| FGFR2 | chr10:123240028 | chr10:124215085 | FGFR2 | ARMS2 |
| ARMS2 | | | | |
| FGFR2 | chr10:123241999 | chr12:53951727 | FGFR2 | ATF7 |
| ATF7 | | | | |
| FGFR2- | chr10:123243016- | chr7:98025564- | FGFR2 | BAIAP2L1 |
| BAIAP2L1 | 123243349 | 98025703 | | |
| FGFR2 | chr10:123240191 | chr10:70499696 | FGFR2 | CCAR1 |
| CCAR1 | | | | |
| FGFR2- | chr10:123241043 | chr10:86165140 | FGFR2 | CCSER2 |
| CCSER2 | | | | |
| FGFR2- | chr10:123242868 | chr15:57824138 | FGFR2 | CGNL1 |
| CGNL1 | | | | |
| FGFR2- | chr10:123239923- | chr5:158518930- | FGFR2 | EBF1 |
| EBF1 | 123240116 | 158519130 | | |
| FGFR2 | chr10:123241189 | chr10:127587833 | FGFR2 | FANK1 |
| FANK1 | | | | |
| FGFR2 | chr3:71189036- | chr10:123242151- | FGFR2 | FOXP1 |
| FOXP1 | 71189121 | 123242274 | | |
| FGFR2 | chr10:123241878- | chr10:75603526- | CAMK2G | FGFR2 |
| CAMK2G | 123241987 | 75603626 | | |
| FGFR2 | chr10:123242192 | chr7:132389880 | FLJ40288 | FGFR2 |
| FLJ40288 | | | | |
| FGFR2- | chr10:123242050- | chr17:7910782- | GUCY2D | FGFR2 |
| GUCY2D | 123242262 | 7911004 | | |
| FGFR2 | chr10:123241258 | chr5:75977315 | IQGAP2 | FGFR2 |
| IQGAP2 | | | | |
| FGFR2- | chr10:123241205 | chr12:80080770 | PAWR | FGFR2 |
| PAWR | | | | |
| FGFR2- | chr10:123242707- | chr3:58121075- | FGFR2 | FLNB |
| | 123242915 | 58121276 | | |
| FLNB | chr10:123240801 | chr3:58143720 | | |
| FGFR2 | chr10:123242151- | chr3:71189036- | FGFR2 | FOXP1 |
| FOXP1 | 123242274 | 71189121 | | |
| FGFR2 | chr10:123241884 | chr2:214016023 | FGFR2 | IKZF2 |
| IKZF2 | | | | |
| FGFR2 | chr10:123242626 | chr1:32497117 | FGFR2 | KHDRBS1 |
| KHDRBS1 | | | | |
| FGFR2- | chr10:123242901 | chr10:75400338 | FGFR2 | MYOZ1 |
| MYOZ1 | | | | |
| FGFR2- | chr10:123257992- | chr10:55668437- | FGFR2 | PCDH15 |
| PCDH15 | 123258171 | 55668600 | | |
| FGFR2- | chr10:123240937 | chr17:66496703 | FGFR2 | PRKAR1A |
| PRKARIA | | | | |
| FGFR2- | chr10:123242694 | chr6:31595369 | FGFR2 | PRRC2A |
| PRRC2A | | | | |
| FGFR2- | chr10:123241892 | chr9:125850348 | FGFR2 | RABGAP1 |
| RABGAP1 | | | | |
| FGFR2- | chr10:123239291 | chr7:12640299 | FGFR2 | SCIN |
| SCIN | | | | |
| FGFR2 | chr10:123240764 | chr20:47762803 | FGFR2 | STAU1 |
| STAU1 | | | | |
| FGFR2 | chr10:123239784 | chr20:43702262 | FGFR2 | STK4 |
| STK4 | | | | |
| FGFR2- | chr10:123242222 | chr4:113200693 | FGFR2 | TIFA |
| TIFA | | | | |
| FGFR2 | chr10:123239464 | chr2:171931100 | FGFR2 | TLK1 |
| TLK1 | | | | |
| FGFR2- | chr10:123240717 | chr2:27516917 | FGFR2 | TRIM54 |
| TRIM54 | | | | |
| FGFR3 | | | | |
| Fusion | | | | |
| Nucleic | | | | |
| Acid | | | | |
| Molecule | Breakpoint 1 | Breakpoint 2 | 5′ Gene | 3′ Gene |
| FGFR3 | chr4:1808575- | chr5:10252744- | FGFR3 | CCT5 |
| CCT5 | 1808796 | 10252900 | | |
| FGFR3- | chr4:1805827- | chr7:135079969- | FGFR3 | CNOT4 |
| CNOT4 | 1805962 | 135080300 | | |
| FGFR3 | chr4:1808845 | chr4:2754775 | FGFR3 | TNIP2 |
| TNIP2 | | | | |
| MET | | | | |
| Fusion | | | | |
| Nucleic | | | | |
| Acid | | | | |
| Molecule | Breakpoint 1 | Breakpoint 2 | 5′ Gene | 3′ Gene |
| MET- | chr7:116411832- | chr11:18429175- | MET | LDHA |
| LDHA | 116411915 | 18429254 | | |
| MET- | chr7:116398519 | chr7:145963989 | CNTNAP2 | MET |
| CNTNAP2 | | | | |
| MET- | chr7:116339469- | chr7:106814702- | HBP1 | MET |
| HBP1 | 116339610 | 106814833 | | |
| MET- | chr7:116412044- | chr19:49598459- | SNRNP70 | MET |
| SNRNP70 | 116412245 | 49598574 | | |
| RAF1 | | | | |
| Fusion | | | | |
| Nucleic | | | | |
| Acid | | | | |
| Molecule | Breakpoint 1 | Breakpoint 2 | 5′ Gene | 3′ Gene |
| RAF1- | chr3:12642793 | chr3:52175928 | POC1A | RAF1 |
| POC1A | | | | |
| RAF1- | chr3:12645756 | chr3:12210717 | SYN2 | RAF1 |
| SYN2 | | | | |
| RAF1- | chr3:12641467 | chr3:15112103 | ZFYVE20 | RAF1 |
| ZFYVE20 | | | | |
| RET | | | | |
| Fusion | | | | |
| Nucleic | | | | |
| Acid | | | | |
| Molecule | Breakpoint 1 | Breakpoint 2 | 5′ Gene | 3′ Gene |
| RET- | chr10:43610900- | chr7:45724501- | RET | ADCY1 |
| ADCY1 | 43611038 | 45724758 | | |
| RET- | chr10:43610040- | chr7:97943769- | BAIAP2L1 | RET |
| BAIAP2L1 | 43610258 | 97943957v | | |
| RET- | chr10:43611694 | chr10:47084409 | RET | NPY4R |
| NPY4R | | | | |
| RET- | chr10:43610049- | chr12:80001152- | RET | PAWR |
| PAWR | 43610142 | 80001299 | | |
| RET- | chr10:43610115 | chr10:45884309 | ALOX5 | RET |
| ALOX5 | | | | |
| RET- | chr10:43610699 | chr10:63843922 | ARID5B | RET |
| ARID5B | | | | |
| RET- | chr10:43610814- | chr10:127554102- | DHX32 | RET |
| DHX32 | 43610918 | 127554221 | | |
| RET- | chr10:43611716 | chr4:120458646 | PDE5A | RET |
| PDE5A | | | | |
| RET- | chr10:43610404 | chr10:64337727 | ZNF365 | RET |
| ZNF365 | | | | |
| ROS1 | | | | |
| Fusion | | | | |
| Nucleic | | | | |
| Acid | | | | |
| Molecule | Breakpoint 1 | Breakpoint 2 | 5′ Gene | 3′ Gene |
| ROS1- | chr6:117650357- | chr17:939714- | ROS1 | ABR |
| ABR | 117650571 | 939902 | | |
| ROS1- | chr6:117648934 | chr6:101139960 | ROS1 | ASCC3 |
| ASCC3 | | | | |
| ROS1- | chr6:117647991 | chr6:80634367 | ROS1 | ELOVL4 |
| ELOVL4 | | | | |
| ROS1- | chr6:117646322 | chr6:163894840 | ROS1 | QKI |
| QKI | | | | |
| ROS1- | chr6:117647122 | chr6:111770010 | ROS1 | REV3L |
| REV3L | | | | |
| ROS1- | chr6:117644495 | chr6:131914175 | MED23 | ROS1 |
| MED23 | | | | |
| ROS1- | chr6:117645825 | chr8:118126691 | SLC30A8 | ROS1 |
| SLC30A8 | | | | |
| ROS1- | chr6:117644675 | chr2:165777489 | SLC38A11 | ROS1 |
| SLC38A11 | | | | |
| ROS1- | chr6:117642549 | chr9:35698416 | TLN1 | ROS1 |
| TLN1 | | | | |
| ROS1- | chr6:117646090 | chr5:149340716 | ROS1 | SLC26A2 |
| SLC26A2 | | | | |
| ROS1- | chr6:117649561 | chr22:39776088 | ROS1 | SYNGR1 |
| SYNGR1 | | | | |
| ROS1- | chr6:117647981 | chr11:101444124 | ROS1 | TRPC6 |
| TRPC6 | | | | |

TABLE 4
Exemplary ALK, BRAF, EGFR, ERBB2, FGFR1, FGFR2, FGFR3, MET, RAF1, RET,
and ROS1 fusion nucleic acid molecules identified in the indicated cancers.

| ALK Fusion Nucleic Acid | | | |
|---|---|---|---|
| Molecule | Cancer | 5′ Gene | 3′ Gene |
| ALK-AGAP1 | prostate acinar | AGAP1 | ALK |
| | adenocarcinoma | | |
| ALK-ARHGEF7 | esophagus carcinoma (NOS) | ARHGEF7 | ALK |
| ALK-BRE | lung non-small cell lung | BRE | ALK |
| | carcinoma (NSCLC) (NOS) | | |
| ALK-EPS8 | lung adenocarcinoma | EPS8 | ALK |
| ALK-GPR113 | prostate acinar | GPR113 | ALK |
| | adenocarcinoma | | |
| ALK-HDAC9 | unknown primary carcinoma | HDAC9 | ALK |
| | (CUP) (NOS) | | |
| ALK-MIPOL1 | colon adenocarcinoma (CRC) | MIPOL1 | ALK |
| ALK-PELI1 | breast (NOS) | PELI1 | ALK |
| ALK-SLC39A10 | breast (NOS) | SLC39A10 | ALK |
| ALK-VKORCIL1 | lung (NOS) | VKORCIL1 | ALK |
| ALK-SORBS1 | lung adenocarcinoma | ALK | SORBS1 |
| ALK-SPINK5 | prostate (NOS) | ALK | SPINK5 |
| BRAF Fusion Nucleic Acid | | | |
| Molecule | Cancer | 5′ Gene | 3′ Gene |
| BRAF-CCDC88C | lung non-small cell lung | CCDC88C | BRAF |
| | carcinoma (NSCLC) (NOS) | | |
| BRAF-COBLL1 | pancreas ductal | COBLL1 | BRAF |
| | adenocarcinoma | | |
| BRAF-CREB3L2 | prostate (NOS) | CREB3L2 | BRAF |
| BRAF-DLC1 | colon adenocarcinoma (CRC) | DLC1 | BRAF |
| BRAF-GOLGA3 | colon adenocarcinoma (CRC) | GOLGA3 | BRAF |
| BRAF-MSI2 | breast (NOS) | MSI2 | BRAF |
| BRAF-TNS3 | soft tissue sarcoma (NOS) | TNS3 | BRAF |
| BRAF-DOCK4 | prostate acinar | BRAF | DOCK4 |
| | adenocarcinoma | | |
| BRAF-RAD51 | prostate acinar | BRAF | RAD51 |
| | adenocarcinoma | | |
| EGFR Fusion Nucleic Acid | | | |
| Molecule | Cancer | 5′ Gene | 3′ Gene |
| EGFR-ABCB1 | lung non-small cell lung | ABCB1 | EGFR |
| | carcinoma (NSCLC) (NOS) | | |
| EGFR-PDE7A | colon adenocarcinoma (CRC) | PDE7A | EGFR |
| EGFR-EZH2 | lung adenocarcinoma | EGFR | EZH2 |
| EGFR-FLJ45974 | colon adenocarcinoma (CRC) | EGFR | FLJ45974 |
| EGFR-ZNF479 | lung adenocarcinoma | EGFR | ZNF479 |
| ERBB2 Fusion Nucleic Acid | | | |
| Molecule | Cancer | 5′ Gene | 3′ Gene |
| ERBB2-FBXL20 | uterus endometrial | FBXL20 | ERBB2 |
| | adenocarcinoma (NOS) | | |
| ERBB2-GRB7 | breast invasive ductal | GRB7 | ERBB2 |
| | carcinoma (IDC) | | |
| ERBB2-MSI2 | gastroesophageal junction | MSI2 | ERBB2 |
| | adenocarcinoma | | |
| ERBB2-RANBP10 | pancreas ductal | RANBP10 | ERBB2 |
| | adenocarcinoma | | |
| ERBB2-SEC14L1 | lung non-small cell lung | SEC14L1 | ERBB2 |
| | carcinoma (NSCLC) (NOS) | | |
| ERBB2-WIPF2 | ovary (NOS) | WIPF2 | ERBB2 |
| ERBB2-GRB7 | breast (NOS) | ERBB2 | GRB7 |
| ERBB2-PRKCA | breast carcinoma (NOS) | ERBB2 | PRKCA |
| FGFR1 Fusion Nucleic Acid | | | |
| Molecule | Cancer | 5′ Gene | 3′ Gene |
| FGFR1-ADAM32 | breast (NOS) | FGFR1 | ADAM32 |
| FGFR1-SLC12A8 | lung non-small cell lung | FGFR1 | SLC12A8 |
| | carcinoma (NSCLC) (NOS) | | |
| FGFR2 Fusion Nucleic Acid | | | |
| Molecule | Cancer | 5′ Gene | 3′ Gene |
| FGFR2-AARSD1 | intra-hepatic | FGFR2 | AARSD1 |
| | cholangiocarcinoma | | |
| FGFR2-ARMS2 | stomach adenocarcinoma | FGFR2 | ARMS2 |
| | (NOS) | | |
| FGFR2-ATF7 | breast (NOS) | FGFR2 | ATF7 |
| FGFR2-BAIAP2L1 | esophagus squamous cell | FGFR2 | BAIAP2L1 |
| | carcinoma (SCC) | | |
| FGFR2-CCAR1 | intra-hepatic | FGFR2 | CCAR1 |
| | cholangiocarcinoma | | |
| FGFR2-CCSER2 | breast invasive ductal | FGFR2 | CCSER2 |
| | carcinoma (IDC) | | |
| FGFR2-CGNL1 | intra-hepatic | FGFR2 | CGNL1 |
| | cholangiocarcinoma | | |
| FGFR2-EBF1 | breast invasive ductal | FGFR2 | EBF1 |
| | carcinoma (IDC) | | |
| FGFR2-FANK1 | stomach adenocarcinoma | FGFR2 | FANK1 |
| | (NOS) | | |
| FGFR2-FOXP1 | ovary (NOS) | FGFR2 | FOXP1 |
| FGFR2-CAMK2G | unknown primary carcinoma | CAMK2G | FGFR2 |
| | (CUP) (NOS) | | |
| FGFR2-FLJ40288 | gastroesophageal junction | FLJ40288 | FGFR2 |
| | adenocarcinoma | | |
| FGFR2-GUCY2D | lung non-small cell lung | GUCY2D | FGFR2 |
| | carcinoma (NSCLC) (NOS) | | |
| FGFR2-IQGAP2 | unknown primary carcinoma | IQGAP2 | FGFR2 |
| | (CUP) (NOS) | | |
| FGFR2-PAWR | unknown primary carcinoma | PAWR | FGFR2 |
| | (CUP) (NOS); unknown | | |
| | primary adenocarcinoma | | |
| FGFR2-FLNB | pancreatobiliary carcinoma | FGFR2 | FLNB |
| | intra-hepatic | | |
| | cholangiocarcinoma | | |
| FGFR2-FOXP1 | ovary (NOS) | FGFR2 | FOXP1 |
| FGFR2-IKZF2 | intra-hepatic | FGFR2 | IKZF2 |
| | cholangiocarcinoma | | |
| FGFR2-KHDRBS1 | intra-hepatic | FGFR2 | KHDRBS1 |
| | cholangiocarcinoma | | |
| FGFR2-MYOZ1 | ovary (NOS) | FGFR2 | MYOZ1 |
| FGFR2-PCDH15 | prostate (NOS) | FGFR2 | PCDH15 |
| FGFR2-PRKAR1A | intra-hepatic | FGFR2 | PRKARIA |
| | cholangiocarcinoma | | |
| FGFR2-PRRC2A | breast carcinoma (NOS) | FGFR2 | PRRC2A |
| FGFR2-RABGAP1 | unknown primary | FGFR2 | RABGAP1 |
| | adenocarcinoma | | |
| FGFR2-SCIN | breast (NOS) | FGFR2 | SCIN |
| FGFR2-STAU1 | intra-hepatic | FGFR2 | STAU1 |
| | cholangiocarcinoma | | |
| FGFR2-STK4 | colon adenocarcinoma (CRC) | FGFR2 | STK4 |
| FGFR2-TIFA | breast invasive ductal | FGFR2 | TIFA |
| | carcinoma (IDC) | | |
| FGFR2-TLK1 | breast carcinoma (NOS) | FGFR2 | TLK1 |
| FGFR2-TRIM54 | unknown primary carcinoma | FGFR2 | TRIM54 |
| | (CUP) (NOS) | | |
| FGFR3 Fusion Nucleic Acid | | | |
| Molecule | Cancer | 5′ Gene | 3′ Gene |
| FGFR3-CCT5 | breast (NOS) | FGFR3 | CCT5 |
| FGFR3-CNOT4 | breast carcinoma (NOS) | FGFR3 | CNOT4 |
| FGFR3-TNIP2 | lung (NOS) | FGFR3 | TNIP2 |
| MET Fusion Nucleic Acid | | | |
| Molecule | Cancer | 5′ Gene | 3′ Gene |
| MET-LDHA | lung non-small cell lung | MET | LDHA |
| | carcinoma (NSCLC) (NOS) | | |
| MET-CNTNAP2 | colon adenocarcinoma (CRC) | CNTNAP2 | MET |
| MET-HBP1 | prostate (NOS) | HBP1 | MET |
| MET-SNRNP70 | colon adenocarcinoma (CRC) | SNRNP70 | MET |
| RAF1 Fusion Nucleic Acid | | | |
| Molecule | Cancer | 5′ Gene | 3′ Gene |
| RAF1-POC1A | prostate acinar | POCIA | RAF1 |
| | adenocarcinoma | | |
| RAF1-SYN2 | colon adenocarcinoma (CRC) | SYN2 | RAF1 |
| RAF1-ZFYVE20 | prostate acinar | ZFYVE20 | RAF1 |
| | adenocarcinoma | | |
| RET Fusion Nucleic Acid | | | |
| Molecule | Cancer | 5′ Gene | 3′ Gene |
| RET-ADCY1 | breast carcinoma (NOS) | RET | ADCY1 |
| RET-BAIAP2L1 | breast (NOS) | BAIAP2L1 | RET |
| RET-NPY4R | colon adenocarcinoma (CRC) | RET | NPY4R |
| RET-PAWR | prostate (NOS) | RET | PAWR |
| RET-ALOX5 | prostate acinar | ALOX5 | RET |
| | adenocarcinoma | | |
| RET-ARID5B | prostate (NOS) | ARID5B | RET |
| RET-DHX32 | lung adenocarcinoma | DHX32 | RET |
| RET-PDE5A | intra-hepatic | PDE5A | RET |
| | cholangiocarcinoma | | |
| RET-ZNF365 | lung (NOS) | ZNF365 | RET |
| ROS1 Fusion Nucleic Acid | | | |
| Molecule | Cancer | 5′ Gene | 3′ Gene |
| ROS1-ABR | unknown primary | ROS1 | ABR |
| | neuroendocrine tumor | | |
| ROS1-ASCC3 | breast (NOS) | ROS1 | ASCC3 |
| ROS1-ELOVL4 | prostate (NOS) | ROS1 | ELOVL4 |
| ROS1-QKI | lung adenocarcinoma | ROS1 | QKI |
| ROS1-REV3L | prostate acinar | ROS1 | REV3L |
| | adenocarcinoma | | |
| ROS1-MED23 | unknown primary carcinoma | MED23 | ROS1 |
| | (CUP) (NOS) | | |
| ROS1-SLC30A8 | lung adenocarcinoma | SLC30A8 | ROS1 |
| ROS1-SLC38A11 | prostate (NOS) | SLC38A11 | ROS1 |
| ROS1-TLN1 | lung non-small cell lung | TLN1 | ROS1 |
| | carcinoma (NSCLC) (NOS) | | |
| ROS1-SLC26A2 | lung adenocarcinoma | ROS1 | SLC26A2 |
| ROS1-SYNGR1 | lung adenocarcinoma | ROS1 | SYNGR1 |
| ROS1-TRPC6 | prostate acinar | ROS1 | TRPC6 |
| | adenocarcinoma | | |

SCC: squamous cell carcinoma;
CUP: carcinoma of unknown primary;
NOS: not otherwise specified;
NSCLC: non-small cell lung cancer;
CRC: colorectal cancer;
IDC: invasive ductal carcinoma

TABLE 5
Exemplary ALK, BRAF, EGFR, ERBB2, FGFR1, FGFR2, FGFR3, MET, RAF1, RET,
and ROS1 fusion nucleic acid molecules and corresponding breakpoints, identified in the
indicated cancers.
ALK Fusions

| ALK | | | | | |
|---|---|---|---|---|---|
| Fusion | | | | | |
| Nucleic | | | | | |
| Acid | | | | | |
| Molecule | Cancer | Breakpoint 1 | Breakpoint 2 | 5′ Gene | 3′ Gene |
| ALK- | prostate acinar | chr2:29446320 | chr2:236984410 | AGAP1 | ALK |
| AGAP1 | adenocarcinoma | | | | |
| ALK | esophagus | chr2:29448192 | chr13:111796690 | ARHGEF7 | ALK |
| ARHGEF7 | carcinoma (NOS) | | | | |
| ALK- | lung non-small | chr2:29448094- | chr2:28431656- | BRE | ALK |
| BRE | cell lung | 29448239 | 28431790 | | |
| | carcinoma | | | | |
| | (NSCLC) (NOS) | | | | |
| ALK- | lung | chr2:29448033 | chr12:15811779 | EPS8 | ALK |
| EPS8 | adenocarcinoma | | | | |
| ALK | prostate acinar | chr2:29448344 | chr2:26546664 | GPR113 | ALK |
| GPR113 | adenocarcinoma | | | | |
| ALK- | unknown primary | chr2:29420593 | chr7:18908783 | HDAC9 | ALK |
| HDAC9 | carcinoma (CUP) | | | | |
| | (NOS) | | | | |
| ALK- | colon | chr2:29446669 | chr14:37906366 | MIPOL1 | ALK |
| MIPOL 1 | adenocarcinoma | | | | |
| | (CRC) | | | | |
| ALK | breast (NOS) | chr2:29448116 | chr2:64360629 | PELI1 | ALK |
| PELI1 | | | | | |
| ALK- | breast (NOS) | chr2:29448047 | chr2:196583393 | SLC39A10 | ALK |
| SLC39A10 | | | | | |
| ALK- | lung (NOS) | chr2:29447360- | chr7:65386539- | VKORC1L1 | ALK |
| VKORCIL | | 29447477 | 65386741 | | |
| 1 | | | | | |
| ALK- | lung | chr2:29447840 | chr10:97287128 | ALK | SORBS1 |
| SORBS1 | adenocarcinoma | | | | |
| ALK- | prostate (NOS) | chr2:29448698 | chr5:147513014 | ALK | SPINK5 |
| SPINK5 | | | | | |

BRAF Fusions

| BRAF | | | | | |
|---|---|---|---|---|---|
| Fusion | | | | | |
| Nucleic | | | | | |
| Acid | | | | | |
| Molecule | Cancer | Breakpoint 1 | Breakpoint 2 | 5′ Gene | 3′ Gene |
| BRAF- | lung non-small | chr7:140489237 | chr14:91742455 | CCDC88C | BRAF |
| CCDC88C | cell lung | | | | |
| | carcinoma | | | | |
| | (NSCLC) (NOS) | | | | |
| BRAF- | pancreas ductal | chr7:140489195 | chr2:165542493 | COBLL1 | BRAF |
| COBLL1 | adenocarcinoma | | | | |
| BRAF- | prostate (NOS) | chr7:140482515 | chr7:137655487 | CREB3L2 | BRAF |
| CREB3L2 | | | | | |
| BRAF | colon | chr7:140434573 | chr8:13242758 | DLC1 | BRAF |
| DLC1 | adenocarcinoma | | | | |
| | (CRC) | | | | |
| BRAF- | colon | chr7:140494157 | chr12:133360988 | GOLGA3 | BRAF |
| GOLGA3 | adenocarcinoma | | | | |
| | (CRC) | | | | |
| BRAF. | breast (NOS) | chr7:140484314 | chr17:55727394 | MSI2 | BRAF |
| MSI2 | | | | | |
| BRAF- | soft tissue | chr7:140483070 | chr7:47407649 | TNS3 | BRAF |
| TNS3 | sarcoma (NOS) | | | | |
| BRAF- | prostate acinar | chr7:140482165 | chr7:111379645 | BRAF | DOCK4 |
| DOCK4 | adenocarcinoma | | | | |
| BRAF- | prostate acinar | chr15:41023127 | chr7:140501694 | BRAF | RAD51 |
| RAD51 | adenocarcinoma | | | | |

EGFR Fusions

| EGFR | | | | | |
|---|---|---|---|---|---|
| Fusion | | | | | |
| Nucleic | | | | | |
| Acid | | | | | |
| Molecule | Cancer | Breakpoint 1 | Breakpoint 2 | 5′ Gene | 3′ Gene |
| EGFR- | lung non-small | chr7:55269027 | chr7:87226070 | ABCB1 | EGFR |
| ABCB1 | cell lung | | | | |
| | carcinoma | | | | |
| | (NSCLC) (NOS) | | | | |
| EGFR- | colon | chr7:55222346 | chr8:66656455 | PDE7A | EGFR |
| PDE7A | adenocarcinoma | | | | |
| | (CRC) | | | | |
| EGFR- | lung | chr7:55268176 | chr7:148565978 | EGFR | EZH2 |
| EZH2 | adenocarcinoma | | | | |
| EGFR- | colon | chr7:55268138 | chr7:53830747 | EGFR | FLJ45974 |
| FLJ45974 | adenocarcinoma | | | | |
| | (CRC) | | | | |
| EGFR- | lung | chr7:55269394 | chr7:57202770 | EGFR | ZNF479 |
| ZNF479 | adenocarcinoma | | | | |

ERBB2 Fusions

| ERBB2 | | | | | |
|---|---|---|---|---|---|
| Fusion | | | | | |
| Nucleic | | | | | |
| Acid | | | | | |
| Molecule | Cancer | Breakpoint 1 | Breakpoint 2 | 5′ Gene | 3′ Gene |
| ERBB2- | uterus endometrial | chr17:37876145- | chr17:37472082- | FBXL20 | ERBB2 |
| FBXL20 | adenocarcinoma | 37876247 | 37472213 | | |
| | (NOS) | | | | |
| ERBB2- | breast invasive | chr17:37881861- | chr17:37896410- | GRB7 | ERBB2 |
| GRB7 | ductal carcinoma | 37882182 | 37896567 | | |
| | (IDC) | | | | |
| ERBB2- | gastroesophageal | chr17:37872528 | chr17:55725868 | MSI2 | ERBB2 |
| MSI2 | junction | | | | |
| | adenocarcinoma | | | | |
| ERBB2- | pancreas ductal | chr17:37865475 | chr16:67790631 | RANBP10 | ERBB2 |
| RANBP10 | adenocarcinoma | | | | |
| ERBB2- | lung non-small | chr17:37879770 | chr17:75181466 | SEC14L1 | ERBB2 |
| SEC14L1 | cell lung | | | | |
| | carcinoma | | | | |
| | (NSCLC) (NOS) | | | | |
| ERBB2- | ovary (NOS) | chr17:37881750 | chr17:38419094 | WIPF2 | ERBB2 |
| WIPF2 | | | | | |
| ERBB2 | breast (NOS) | chr17:37881373- | chr17:37902966- | ERBB2 | GRB7 |
| GRB7 | | 37881485 | 37903081 | | |
| ERBB2- | breast carcinoma | chr17:37872883 | chr17:64588788 | ERBB2 | PRKCA |
| PRKCA | (NOS) | | | | |

FGFR1 Fusions

| FGFR1 | | | | | |
|---|---|---|---|---|---|
| Fusion | | | | | |
| Nucleic | | | | | |
| Acid | | | | | |
| Molecule | Cancer | Breakpoint 1 | Breakpoint 2 | 5′ Gene | 3′ Gene |
| FGFR1- | breast (NOS) | chr8:38271185 | chr8:39031195 | FGFR1 | ADAM32 |
| ADAM32 | | | | | |
| FGFR1- | lung non-small | chr8:38285341 | chr3:124897236 | FGFR1 | SLC12A8 |
| SLC12A8 | cell lung | | | | |
| | carcinoma | | | | |
| | (NSCLC) (NOS) | | | | |

FGFR2 Fusions

| FGFR2 | | | | | |
|---|---|---|---|---|---|
| Fusion | | | | | |
| Nucleic | | | | | |
| Acid | | | | | |
| Molecule | Cancer | Breakpoint 1 | Breakpoint 2 | 5′ Gene | 3′ Gene |
| FGFR2- | intra-hepatic | chr10:123240088 | chr17:41114482 | FGFR2 | AARSD1 |
| AARSD1 | cholangiocarcino | | | | |
| | ma | | | | |
| FGFR2- | stomach | chr10:123240028 | chr10:124215085 | FGFR2 | ARMS2 |
| ARMS2 | adenocarcinoma | | | | |
| | (NOS) | | | | |
| FGFR2- | breast (NOS) | chr10:123241999 | chr12:53951727 | FGFR2 | ATF7 |
| ATF7 | | | | | |
| FGFR2- | esophagus | chr10:123243016- | chr7:98025564- | FGFR2 | BAIAP2L1 |
| BAIAP2L1 | squamous cell | 123243349 | 98025703 | | |
| | carcinoma (SCC) | | | | |
| FGFR2- | intra-hepatic | chr10:123240191 | chr10:70499696 | FGFR2 | CCAR1 |
| CCAR1 | cholangiocarcinoma | | | | |
| FGFR2- | breast invasive | chr10:123241043 | chr10:86165140 | FGFR2 | CCSER2 |
| CCSER2 | ductal carcinoma | | | | |
| | (IDC) | | | | |
| FGFR2- | intra-hepatic | chr10:123242868 | chr15:57824138 | FGFR2 | CGNL1 |
| CGNL1 | cholangiocarcinoma | | | | |
| FGFR2- | breast invasive | chr10:123239923- | chr5:158518930- | FGFR2 | EBF1 |
| EBF1 | ductal carcinoma | 123240116 | 158519130 | | |
| | (IDC) | | | | |
| FGFR2- | stomach | chr10:123241189 | chr10:127587833 | FGFR2 | FANK1 |
| FANK1 | adenocarcinoma | | | | |
| | (NOS) | | | | |
| FGFR2- | ovary (NOS) | chr3:71189036- | chr10:123242151- | FGFR2 | FOXP1 |
| FOXP1 | | 71189121 | 123242274 | | |
| FGFR2- | unknown primary | chr10:123241878- | chr10:75603526- | CAMK2G | FGFR2 |
| CAMK2G | carcinoma (CUP) | 123241987 | 75603626 | | |
| | (NOS) | | | | |
| FGFR2- | gastroesophageal | chr10:123242192 | chr7:132389880 | FLJ40288 | FGFR2 |
| FLJ40288 | junction | | | | |
| | adenocarcinoma | | | | |
| FGFR2- | lung non-small | chr10:123242050- | chr17:7910782- | GUCY2D | FGFR2 |
| GUCY2D | cell lung | 123242262 | 7911004 | | |
| | carcinoma | | | | |
| | (NSCLC) (NOS) | | | | |
| FGFR2- | unknown primary | chr10:123241258 | chr5:75977315 | IQGAP2 | FGFR2 |
| IQGAP2 | carcinoma (CUP) | | | | |
| | (NOS) | | | | |
| FGFR2- | unknown primary | chr10:123241205 | chr12:80080770 | PAWR | FGFR2 |
| PAWR | carcinoma (CUP) | | | | |
| | (NOS); unknown | | | | |
| | primary | | | | |
| | adenocarcinoma | | | | |
| FGFR2- | pancreatobiliary | chr10:123242707- | chr3:58121075- | FGFR2 | FLNB |
| FLNB | carcinoma | 123242915 | 58121276 | | |
| | intra-hepatic | chr10:123240801 | chr3:58143720 | | |
| | cholangiocarcinoma | | | | |
| FGFR2- | ovary (NOS) | chr10:123242151- | chr3:71189036- | FGFR2 | FOXP1 |
| FOXP1 | | 123242274 | 71189121 | | |
| FGFR2- | intra-hepatic | chr10:123241884 | chr2:214016023 | FGFR2 | IKZF2 |
| IKZF2 | cholangiocarcinoma | | | | |
| FGFR2- | intra-hepatic | chr10:123242626 | chr1:32497117 | FGFR2 | KHDRBS1 |
| KHDRBS1 | cholangiocarcinoma | | | | |
| FGFR2- | ovary (NOS) | chr10:123242901 | chr10:75400338 | FGFR2 | MYOZ1 |
| MYOZ1 | | | | | |
| FGFR2- | prostate (NOS) | chr10:123257992- | chr10:55668437- | FGFR2 | PCDH15 |
| PCDH15 | | 123258171 | 55668600 | | |
| FGFR2- | intra-hepatic | chr10:123240937 | chr17:66496703 | FGFR2 | PRKAR1A |
| PRKAR1A | cholangiocarcinoma | | | | |
| FGFR2- | breast carcinoma | chr10:123242694 | chr6:31595369 | FGFR2 | PRRC2A |
| PRRC2A | (NOS) | | | | |
| FGFR2- | unknown primary | chr10:123241892 | chr9:125850348 | FGFR2 | RABGAP1 |
| RABGAP1 | adenocarcinoma | | | | |
| FGFR2- | breast (NOS) | chr10:123239291 | chr7:12640299 | FGFR2 | SCIN |
| SCIN | | | | | |
| FGFR2 | intra-hepatic | chr10:123240764 | chr20:47762803 | FGFR2 | STAU1 |
| STAU1 | cholangiocarcinoma | | | | |
| FGFR2- | colon | chr10:123239784 | chr20:43702262 | FGFR2 | STK4 |
| STK4 | adenocarcinoma | | | | |
| | (CRC) | | | | |
| FGFR2 | breast invasive | chr10:123242222 | chr4:113200693 | FGFR2 | TIFA |
| TIFA | ductal carcinoma | | | | |
| | (IDC) | | | | |
| FGFR2 | breast carcinoma | chr10:123239464 | chr2:171931100 | FGFR2 | TLK1 |
| TLK1 | (NOS) | | | | |
| FGFR2 | unknown primary | chr10:123240717 | chr2:27516917 | FGFR2 | TRIM54 |
| TRIM54 | carcinoma (CUP) | | | | |
| | (NOS) | | | | |

FGFR3 Fusions

| FGFR3 | | | | | |
|---|---|---|---|---|---|
| Fusion | | | | | |
| Nucleic | | | | | |
| Acid | | | | | |
| Molecule | Cancer | Breakpoint 1 | Breakpoint 2 | 5′ Gene | 3′ Gene |
| FGFR3 | breast (NOS) | chr4:1808575- | chr5:10252744- | FGFR3 | CCT5 |
| CCT5 | | 1808796 | 10252900 | | |
| FGFR3- | breast carcinoma | chr4:1805827- | chr7:135079969- | FGFR3 | CNOT4 |
| CNOT4 | (NOS) | 1805962 | 135080300 | | |
| FGFR3 | lung (NOS) | chr4:1808845 | chr4:2754775 | FGFR3 | TNIP2 |
| TNIP2 | | | | | |

MET Fusions

| MET | | | | | |
|---|---|---|---|---|---|
| Fusion | | | | | |
| Nucleic | | | | | |
| Acid | | | | | |
| Molecule | Cancer | Breakpoint 1 | Breakpoint 2 | 5′ Gene | 3′ Gene |
| MET- | lung non-small | chr7:116411832- | chr11:18429175- | MET | LDHA |
| LDHA | cell lung | 116411915 | 18429254 | | |
| | carcinoma | | | | |
| | (NSCLC) (NOS) | | | | |
| MET- | colon | chr7:116398519 | chr7:145963989 | CNTNAP2 | MET |
| CNTNAP2 | adenocarcinoma | | | | |
| | (CRC) | | | | |
| MET- | prostate (NOS) | chr7:116339469- | chr7:106814702- | HBP1 | MET |
| HBP1 | | 116339610 | 106814833 | | |
| MET- | colon | chr7:116412044- | chr19:49598459- | SNRNP70 | MET |
| SNRNP70 | adenocarcinoma | 116412245 | 49598574 | | |
| | (CRC) | | | | |

RAF1 Fusions

| RAF1 | | | | | |
|---|---|---|---|---|---|
| Fusion | | | | | |
| Nucleic | | | | | |
| Acid | | | | | |
| Molecule | Cancer | Breakpoint 1 | Breakpoint 2 | 5′ Gene | 3′ Gene |
| RAF1- | prostate acinar | chr3:12642793 | chr3:52175928 | POC1A | RAF1 |
| POC1A | adenocarcinoma | | | | |
| RAF1- | colon | chr3:12645756 | chr3:12210717 | SYN2 | RAF1 |
| SYN2 | adenocarcinoma | | | | |
| | (CRC) | | | | |
| RAF1- | prostate acinar | chr3:12641467 | chr3:15112103 | ZFYVE20 | RAF1 |
| ZFYVE20 | adenocarcinoma | | | | |

RET Fusions

| RET | | | | | |
|---|---|---|---|---|---|
| Fusion | | | | | |
| Nucleic | | | | | |
| Acid | | | | | |
| Molecule | Cancer | Breakpoint 1 | Breakpoint 2 | 5′ Gene | 3′ Gene |
| RET | breast carcinoma | chr10:43610900- | chr7:45724501- | RET | ADCY1 |
| ADCY1 | (NOS) | 43611038 | 45724758 | | |
| RET- | breast (NOS) | chr10:43610040- | chr7:97943769- | BAIAP2L1 | RET |
| BAIAP2L1 | | 43610258 | 97943957 | | |
| RET- | colon | chr10:43611694 | chr10:47084409 | RET | NPY4R |
| NPY4R | adenocarcinoma | | | | |
| | (CRC) | | | | |
| RET- | prostate (NOS) | chr10:43610049- | chr12:80001152- | RET | PAWR |
| PAWR | | 43610142 | 80001299 | | |
| RET- | prostate acinar | chr10:43610115 | chr10:45884309 | ALOX5 | RET |
| ALOX5 | adenocarcinoma | | | | |
| RET- | prostate (NOS) | chr10:43610699 | chr10:63843922 | ARID5B | RET |
| ARID5B | | | | | |
| RET- | lung | chr10:43610814- | chr10:127554102- | DHX32 | RET |
| DHX32 | adenocarcinoma | 43610918 | 127554221 | | |
| RET- | intra-hepatic | chr10:43611716 | chr4:120458646 | PDE5A | RET |
| PDE5A | cholangiocarcinoma | | | | |
| RET- | lung (NOS) | chr10:43610404 | chr10:64337727 | ZNF365 | RET |
| ZNF365 | | | | | |

ROS1 Fusions

| ROS1 | | | | | |
|---|---|---|---|---|---|
| Fusion | | | | | |
| Nucleic | | | | | |
| Acid | | | | | |
| Molecule | Cancer | Breakpoint 1 | Breakpoint 2 | 5′ Gene | 3′ Gene |
| ROS1- | unknown primary | chr6:117650357- | chr17:939714- | ROS1 | ABR |
| ABR | neuroendocrine | 117650571 | 939902 | | |
| | tumor | | | | |
| ROS1- | breast (NOS) | chr6:117648934 | chr6:101139960 | ROS1 | ASCC3 |
| ASCC3 | | | | | |
| ROS1- | prostate (NOS) | chr6:117647991 | chr6:80634367 | ROS1 | ELOVL4 |
| ELOVL4 | | | | | |
| ROS1- | lung | chr6:117646322 | chr6:163894840 | ROS1 | QKI |
| QKI | adenocarcinoma | | | | |
| ROS1- | prostate acinar | chr6:117647122 | chr6:111770010 | ROS1 | REV3L |
| REV3L | adenocarcinoma | | | | |
| ROS1- | unknown primary | chr6:117644495 | chr6:131914175 | MED23 | ROS1 |
| MED23 | carcinoma (CUP) | | | | |
| | (NOS) | | | | |
| ROS1- | lung | chr6:117645825 | chr8:118126691 | SLC30A8 | ROS1 |
| SLC30A8 | adenocarcinoma | | | | |
| ROS1- | prostate (NOS) | chr6:117644675 | chr2:165777489 | SLC38A11 | ROS1 |
| SLC38A11 | | | | | |
| ROS1- | lung non-small | chr6:117642549 | chr9:35698416 | TLN1 | ROS1 |
| TLN1 | cell lung | | | | |
| | carcinoma | | | | |
| | (NSCLC) (NOS) | | | | |
| ROS1- | lung | chr6:117646090 | chr5:149340716 | ROS1 | SLC26A2 |
| SLC26A2 | adenocarcinoma | | | | |
| ROS1- | lung | chr6:117649561 | chr22:39776088 | ROS1 | SYNGR1 |
| SYNGR1 | adenocarcinoma | | | | |
| ROS1- | prostate acinar | chr6:117647981 | chr11:101444124 | ROS1 | TRPC6 |
| TRPC6 | adenocarcinoma | | | | |

SCC: squamous cell carcinoma;
CUP: carcinoma of unknown primary;
NOS: not otherwise specified;
NSCLC: non-small cell lung cancer;
CRC: colorectal cancer;
IDC: invasive ductal carcinoma

TABLE 6
Exemplary ALK, BRAF, ERBB2, FGFR1, FGFR2, FGFR3, MET,
NTRK1, RAF1, RET, and ROS1 fusion nucleic acid molecules and
corresponding breakpoints, identified in the indicated cancer types.
ALK Fusions

| ALK Fusion | | | | | |
|---|---|---|---|---|---|
| Nucleic Acid | | | | | |
| Molecule | Cancer | Breakpoint 1 | Breakpoint 2 | 5′ Gene | 3′ Gene |
| ALK - | lung squamous | chr2: 29447157 | chr2: 109110640 | GCC2 | ALK |
| GCC2 | cell carcinoma | | | | |
| | (SCC) | | | | |
| ALK - HIP1 | unknown | chr2: 29447938- | chr7: 75171619- | HIP1 | ALK |
| | primary | 29448138 | 75171750 | | |
| | carcinoma | | | | |
| | (CUP) (NOS) | | | | |
| ALK - | pancreas (NOS) | chr2: 29449486- | chr9: 723093- | KANK1 | ALK |
| KANK1 | | 29449674 | 723285 | | |
| ALK - KLC1 | unknown | chr2: 29448020- | chr14: 104141458- | KLC1 | ALK |
| | primary | 29448230 | 104141626 | | |
| | adenocarcinoma | | | | |
| ALK - | unknown | chr2: 29447358- | chr12: 27813429- | PPFIBP1 | ALK |
| PPFIBP1 | primary (NOS) | 29447625 | 27813685 | | |
| ALK - | lung non-small | chr2: 29448437- | chr11: 16803216- | PLEKHA7 | ALK |
| PLEKHA7 | cell lung | 29448765 | 16803516 | | |
| | carcinoma | | | | |
| | (NSCLC) (NOS) | | | | |
| ALK - TFG | breast carcinoma | chr2: 29449097 | chr3: 100450538 | TFG | ALK |
| | (NOS) | | | | |
| ALK - | pancreas ductal | chr2: 29448196 | chr1: 154135477 | TPM3 | ALK |
| TPM3 | adenocarcinoma | | | | |

BRAF Fusions

| BRAF | | | | | |
|---|---|---|---|---|---|
| Fusion | | | | | |
| Nucleic Acid | | | | | |
| Molecule | Cancer | Breakpoint 1 | Breakpoint 2 | 5′ Gene | 3′ Gene |
| BRAF - | colon | chr7: 140493457 | chr7: 91701220 | AKAP9 | BRAF |
| AKAP9 | adenocarcinoma | | | | |
| | (CRC) | | | | |
| BRAF - | esophagus | chr7: 140481643 | chr7: 102730767 | ARMC10 | BRAF |
| ARMC10 | adenocarcinoma | | | | |
| BRAF | colon | chr7: 140487304 | chr7: 140243015 | DENND2A | BRAF |
| DENND2A | adenocarcinoma | | | | |
| | (CRC) | | | | |
| BRAF - | prostate acinar | chr7: 140488072 | chr7: 139800753 | JHDM1D | BRAF |
| JHDM1D | adenocarcinoma | | | | |
| BRAF - | prostate acinar | chr7: 140485311 | chr7: 138564061 | KIAA1549 | BRAF |
| KIAA1549 | adenocarcinoma | | | | |
| BRAF | rectum | chr7: 140481605 | chr7: 140157871 | MKRN1 | BRAF |
| MKRN1 | adenocarcinoma | | | | |
| | (CRC) | | | | |
| BRAF - | colon | chr7: 140485113 | chr7: 129373037 | NRF1 | BRAF |
| NRF1 | adenocarcinoma | | | | |
| | (CRC) | | | | |
| BRAF - | prostate acinar | chr7: 140499628 | chr1: 205639559 | SLC45A3 | BRAF |
| SLC45A3 | adenocarcinoma | | | | |
| BRAF | prostate acinar | chr7: 140487680 | chr7: 127715943 | SND1 | BRAF |
| SND1 | adenocarcinoma | | | | |
| | prostate ductal | chr7: 140491295 | chr7: 127399380 | | |
| | adenocarcinoma | | | | |
| | colon | chr7: 140481458- | chr7: 127356978- | | |
| | adenocarcinoma | 140481757 | 127357015 | | |
| | (CRC) | | | | |
| BRAF - | rectum | chr7: 140490521 | chr7: 138224175 | BRAF | TRIM24 |
| TRIM24 | adenocarcinoma | | | | |
| | (CRC) | | | | |
| BRAF - | colon | chr7: 140485942 | chr7: 138765898 | ZC3HAV1 | BRAF |
| ZC3HAV1 | adenocarcinoma | | | | |
| | (CRC) | | | | |
| BRAF - | lung | chr7: 140481495 | chr7: 111929341 | ZNF277 | BRAF |
| ZNF277 | adenocarcinoma | | | | |

ERBB2 Fusions

| ERBB2 | | | | | |
|---|---|---|---|---|---|
| Fusion | | | | | |
| Nucleic Acid | | | | | |
| Molecule | Cancer | Breakpoint 1 | Breakpoint 2 | 5′ Gene | 3′ Gene |
| ERBB2 - | colon | chr17: 37883936 | chr17: 37789550 | ERBB2 | PPP1R1B |
| PPP1R1B | adenocarcinoma | | | | |
| | (CRC) | | | | |

FGFR1 Fusions

| FGFR1 | | | | | |
|---|---|---|---|---|---|
| Fusion | | | | | |
| Nucleic Acid | | | | | |
| Molecule | Cancer | Breakpoint 1 | Breakpoint 2 | 5′ Gene | 3′ Gene |
| FGFR1 - | cervix squamous | chr8: 38271241 | chr8: 39537661 | ADAM18 | FGFR1 |
| ADAM18 | cell carcinoma | | | | |
| | (SCC) | | | | |
| FGFR1 - | breast (NOS) | chr8: 38272085- | chr8: 38036460- | BAG4 | FGFR1 |
| BAG4 | | 38272258 | 38036578 | | |
| FGFR1 - | breast invasive | chr8: 38273440 | chr8: 38622825 | FGFR1 | TACC1 |
| TACC1 | lobular | | | | |
| | carcinoma (ILC) | | | | |

FGFR2 Fusions

| FGFR2 | | | | | |
|---|---|---|---|---|---|
| Fusion | | | | | |
| Nucleic Acid | | | | | |
| Molecule | Cancer | Breakpoint 1 | Breakpoint 2 | 5′ Gene | 3′ Gene |
| FGFR2 - | lung large cell | chr10: 123240839 | chr11: 34921475 | FGFR2 | APIP |
| APIP | carcinoma | | | | |
| FGFR2 - | stomach | chr10: 123241676 | chr10: 123551234 | FGFR2 | ATE1 |
| ATE1 | adenocarcinoma | | | | |
| | (NOS) | | | | |
| FGFR2 - | unknown | chr10: 123241383 | chr10: 60410103 | FGFR2 | BICC1 |
| BICC1 | primary | | | | |
| | carcinoma | | | | |
| | (CUP) (NOS) | | | | |
| | pancreatobiliary | chr10: 123241708 | chr10: 60428801 | | |
| | carcinoma | | | | |
| | lung | chr10: 123239702 | chr10: 60429695 | | |
| | adenocarcinoma | | | | |
| FGFR2 | prostate (NOS) | chr10: 123240329 | chr7: 115606014 | TFEC | FGFR2 |
| TFEC | | | | | |
| FGFR2 - | intra-hepatic | chr10: 123241875 | chr17: 73384322 | FGFR2 | GRB2 |
| GRB2 | cholangiocar- | | | | |
| | cinoma | | | | |
| | breast invasive | chr10: 123242159 | chr17: 73365008 | | |
| | ductal carcinoma | | | | |
| | (IDC) | | | | |
| FGFR2 - | gallbladder | chr10: 123241180 | chr10: 24605241 | FGFR2 | KIAA1217 |
| KIAA1217 | adenocarcinoma | | | | |
| FGFR2 - | lung | chr10: 123242758- | chr10: 118708900- | FGFR2 | KIAA1598 |
| KIAA1598 | adenocarcinoma | 123242929 | 118709045 | | |
| FGFR2 - | pancreatobiliary | chr10: 123241285 | chr1: 39918104 | FGFR2 | MACF1 |
| MACF1 | carcinoma | | | | |
| FGFR2- | unknown | chr10: 123240474- | chr22: 36695796- | FGFR2 | MYH9 |
| MYH9 | primary | 123240649 | 36695923 | | |
| | adenocarcinoma | | | | |
| FGFR2 - | lung squamous | chr10: 123241635 | chr10: 115380780 | FGFR2 | NRAP |
| NRAP | cell carcinoma | | | | |
| | (SCC) | | | | |
| FGFR2 - | breast carcinoma | chr10: 123241293 | chr10: 112580773 | FGFR2 | RBM20 |
| RBM20 | (NOS) | | | | |
| FGFR2 - | intra-hepatic | chr10: 123239840- | chr3: 113207848- | FGFR2 | SPICE1 |
| SPICE1 | cholangio- | 123239995 | 113207958 | | |
| | carcinoma | | | | |
| FGFR2 - | pancreatobiliary | chr10: 123239675- | chr10: 123988441- | FGFR2 | TACC2 |
| TACC2 | carcinoma | 123239796 | 123988546 | | |
| | unknown | chr10: 123241654- | chr10: 123989561- | | |
| | primary | 123241788 | 123989655 | | |
| | adenocarcinoma | | | | |
| | lung small cell | chr10: 123239631 | chr10: 123988734 | | |
| | undifferentiated | | | | |
| | carcinoma | | | | |
| | breast invasive | chr10: 123241742 | chr10: 123987953 | | |
| | ductal carcinoma | | | | |
| | (IDC) | | | | |
| FGFR2 | stomach | chr10: 123239845 | chr10: 114543839 | FGFR2 | VTI1A |
| VTI1A | adenocarcinoma | | | | |
| | (NOS) | | | | |
| FGFR2 - | lung | chr10: 123242151- | chr10: 28905673- | FGFR2 | WAC |
| WAC | adenocarcinoma | 123242331 | 28905852 | | |
| FGFR2 - | intra-hepatic | chr10: 123239968 | chr14: 100834590 | FGFR2 | WARS |
| WARS | cholangio- | | | | |
| | carcinoma | | | | |
| | lung non-small | chr10: 123242027- | chr14: 100829090- | | |
| | cell lung | 123242147 | 100829181 | | |
| | carcinoma | | | | |
| | (NSCLC) (NOS) | | | | |
| FGFR2 - | unknown | chr10: 123241846 | chr1: 35851725 | FGFR2 | ZMYM4 |
| ZMYM4 | primary | | | | |
| | carcinoma | | | | |
| | (CUP) (NOS) | | | | |

FGFR3 Fusions

| FGFR3 | | | | | |
|---|---|---|---|---|---|
| Fusion | | | | | |
| Nucleic Acid | | | | | |
| Molecule | Cancer | Breakpoint 1 | Breakpoint 2 | 5′ Gene | 3′ Gene |
| FGFR3 - | lung non-small | chr4: 1808801 | chr4: 2882826 | FGFR3 | ADD1 |
| ADD1 | cell lung | | | | |
| | carcinoma | | | | |
| | (NSCLC) (NOS) | | | | |
| FGFR3 - | pancreas (NOS) | chr4: 1808551 | chr14: 106005444 | FGFR3 | IGH |
| IGH | | | | | |
| FGFR3 - | small intestine | chr4: 1808563- | chr4: 1730156- | FGFR3 | TACC3 |
| TACC3 | adenocarcinoma | 1808711 | 1730288 | | |
| | prostate acinar | chr4: 1808845 | chr4: 1739165 | | |
| | adenocarcinoma | | | | |
| | colon | chr4: 1808751 | chr4: 1739667 | | |
| | adenocarcinoma | | | | |
| | (CRC) | | | | |
| | unknown | chr4: 1808710 | chr4: 1740755 | | |
| | primary | | | | |
| | squamous cell | | | | |
| | carcinoma (SCC) | | | | |
| | breast invasive | chr4: 1808729 | chr4: 1739043 | | |
| | lobular | | | | |
| | carcinoma (ILC) | | | | |
| | eye intraocular | chr4: 1808705 | chr4: 1739261 | | |
| | melanoma | | | | |
| | bladder | chr4: 1808678 | chr4: 1738998 | | |
| | adenocarcinoma | | | | |
| | lung small cell | chr4: 1808911 | chr4: 1739886 | | |
| | undifferentiated | | | | |
| | carcinoma | | | | |
| | kidney (NOS) | chr4: 1808671 | chr4: 1741144 | | |
| FGFR3 - | prostate acinar | chr4: 1808442- | chr4: 1949176- | FGFR3 | WHSC1 |
| WHSC1 | adenocarcinoma | 1808709 | 1949445 | | |

MET Fusions

| MET Fusion | | | | | |
|---|---|---|---|---|---|
| Nucleic Acid | | | | | |
| Molecule | Cancer | Breakpoint 1 | Breakpoint 2 | 5′ Gene | 3′ Gene |
| MET - | colon | chr7: 116435955 | chr7: 116506109 | MET | CAPZA2 |
| CAPZA2 | adenocarcinoma | | | | |
| | (CRC) | | | | |
| MET - ST7 | colon | chr7: 116399431 | chr7: 116726687 | ST7 | MET |
| | adenocarcinoma | | | | |
| | (CRC) | | | | |
| | skin melanoma | chr7: 116399373 | chr7: 116803090 | | |

NTRK1 Fusions

| NTRK1 | | | | | |
|---|---|---|---|---|---|
| Fusion | | | | | |
| Nucleic Acid | | | | | |
| Molecule | Cancer | Breakpoint 1 | Breakpoint 2 | 5′ Gene | 3′ Gene |
| NTRK1 - | prostate (NOS) | chr1: 156843853 | chr1: 156441602 | NTRK1 | MEF2D |
| MEF2D | | | | | |

RAF1 Fusions

| RAF1 | | | | | |
|---|---|---|---|---|---|
| Fusion | | | | | |
| Nucleic Acid | | | | | |
| Molecule | Cancer | Breakpoint 1 | Breakpoint 2 | 5′ Gene | 3′ Gene |
| RAF1 - | colon | chr3: 12645483 | chr3: 42231967 | RAF1 | TRAK1 |
| TRAK1 | adenocarcinoma | | | | |
| | (CRC) | | | | |

RET Fusions

| RET Fusion | | | | | |
|---|---|---|---|---|---|
| Nucleic Acid | | | | | |
| Molecule | Cancer | Breakpoint 1 | Breakpoint 2 | 5′ Gene | 3′ Gene |
| RET - | esophagus | chr10: 43609615- | chr10: 43651937- | RET | CSGALNACT2 |
| CSGALNAC | squamous cell | 43609862 | 43652250 | | |
| T2 | carcinoma (SCC) | | | | |
| RET - | lung non-small | chr10: 43611481 | chr14: 67585496 | RET | GPHN |
| GPHN | cell lung | | | | |
| | carcinoma | | | | |
| | (NSCLC) (NOS) | | | | |
| RET - | bladder | chr10: 43611605- | chr10: 51586055- | NCOA4 | RET |
| NCOA4 | urothelial | 43611729 | 51586121 | | |
| | (transitional cell) | | | | |
| | carcinoma | | | | |
| | unknown | chr10: 43611515 | chr10: 51588839 | | |
| | primary | | | | |
| | carcinoma | | | | |
| | (CUP) (NOS) | | | | |
| RET - | colon | chr10: 43597874 | chr10: 43705242 | RET | RASGEF1A |
| RASGEF1A | adenocarcinoma | | | | |
| | (CRC) | | | | |
| RET - | colon | chr10: 43610721 | chr10: 61653627 | CCDC6 | RET |
| CCDC6 | adenocarcinoma | | | | |
| | (CRC) | | | | |
| | rectum | chr10: 43610931 | chr10: 61639079 | | |
| | adenocarcinoma | | | | |
| | (CRC) | | | | |
| RET - ERC1 | soft tissue | chr10: 43610879 | chr12: 1346557 | ERC1 | RET |
| | sarcoma (NOS) | | | | |
| RET - | colon | chr10: 43610513- | chr10: 24813557- | KIAA1217 | RET |
| KIAA1217 | adenocarcinoma | 43610815 | 24813833 | | |
| | (CRC) | | | | |
| | esophagus | chr10: 43611407 | chr10: 24816256 | | |
| | adenocarcinoma | | | | |
| | lung non-small | chr10: 43610121 | chr10: 24816841 | | |
| | cell lung | | | | |
| | carcinoma | | | | |
| | (NSCLC) (NOS) | | | | |
| RET - | breast (NOS) | chr10: 43610514- | chr10: 32313549- | KIF5B | RET |
| KIF5B | | 43610659 | 32313707 | | |
| | lung squamous | chr10: 43609418 | chr10: 32304634 | | |
| | cell carcinoma | | | | |
| | (SCC) | | | | |
| | unknown | chr10: 43611132- | chr10: 32312409- | | |
| | primary | 43611473 | 32312729 | | |
| | adenocarcinoma | | | | |
| RET - | lung | chr10: 43611376- | chr7: 138250812- | TRIM24 | RET |
| TRIM24 | adenocarcinoma | 43611543 | 138251052 | | |
| RET - VCL | lung | chr10: 43611112- | chr10: 75861515- | VCL | RET |
| | adenocarcinoma | 43611316 | 75861716 | | |

ROS1 Fusions

| ROS1 | | | | | |
|---|---|---|---|---|---|
| Fusion | | | | | |
| Nucleic Acid | | | | | |
| Molecule | Cancer | Breakpoint 1 | Breakpoint 2 | 5′ Gene | 3′ Gene |
| ROS1 - EZR | unknown | chr6: 117645920- | chr6: 159191019- | EZR | ROS1 |
| | primary | 117646133 | 159191124 | | |
| | carcinoma | | | | |
| | (CUP) (NOS) | | | | |
| ROS1 - | appendix | chr6: 117643144 | chr6: 117886533 | GOPC | ROS1 |
| GOPC | adenocarcinoma | | | | |
| | liver | chr6: 117643807 | chr6: 117885081 | | |
| | hepatocellular | | | | |
| | carcinoma | | | | |
| | (HCC) | | | | |
| | rectum | chr6: 117641972 | chr6: 117894977 | | |
| | adenocarcinoma | | | | |
| | (CRC) | | | | |
| | unknown | chr6: 117645121 | chr6: 117885917 | | |
| | primary | | | | |
| | neuroendocrine | | | | |
| | tumor | | | | |
| ROS1 - | lung | chr6: 117642709- | chr15: 52512166- | MYO5C | ROS1 |
| MYO5C | adenocarcinoma | 117642959 | 52512328 | | |
| ROS1 - | prostate acinar | chr6: 117647493 | chr6: 125568358 | ROS1 | TPD52L1 |
| TPD52L1 | adenocarcinoma | | | | |

SCC: squamous cell carcinoma;
CUP: carcinoma of unknown primary;
NOS: not otherwise specified;
NSCLC: non-small cell lung cancer;
CRC: colorectal cancer;
ILC: invasive lobular breast cancer;
IDC: invasive ductal carcinoma;
HCC: hepatocellular carcinoma

In some embodiments of any of the fusion nucleic acid molecules provided herein, the fusion nucleic acid molecule is a genomic nucleic acid molecule (i.e., genomic DNA or fragments thereof), or a transcribed nucleic acid molecule, e.g., an RNA such as mRNA, or a cDNA, or fragments thereof.

In some embodiments of any of the fusion nucleic acid molecules provided herein, the chromosomal coordinates corresponding to any of the breakpoints described herein correspond to Homo sapiens (human) genome assembly GRCh37 (hg19).

### (ii) Kinase Fusion Polypeptides

In certain aspects, provided herein are ALK, BRAF, EGFR, ERBB2, FGFR1, FGFR2, FGFR3, MET, NTRK1, RAF1, RET, or ROS1 fusion polypeptides which comprise at least a portion of an ALK, BRAF, EGFR, ERBB2, FGFR1, FGFR2, FGFR3, MET, NTRK1, RAF1, RET, or ROS1 polypeptide and at least a portion of a polypeptide encoded by another gene. In some embodiments, a fusion polypeptide of the disclosure is a fusion polypeptide encoded by any of the ALK, BRAF, EGFR, ERBB2, FGFR1, FGFR2, FGFR3, MET, NTRK1, RAF1, RET, or ROS1 fusion nucleic acid molecules provided herein, or a portion thereof.

In some aspects, provided herein are ALK fusion polypeptides that comprise at least a portion of an ALK polypeptide and at least a portion of a polypeptide encoded by another gene, e.g., an AGAP1, ARHGEF7, BRE, EPS8, GPR113, HDAC9, MIPOL1, PELI1, SLC39A10, VKORC1L1, PLEKHA7, SPINK5, GCC2, HIP1, KANK1, KLC1, PPFIBP1, SORBS1, TFG, or TPM3 polypeptide. For example, in some embodiments, provided herein are fusion polypeptides encoded by an AGAP1-ALK, ARHGEF7-ALK, BRE-ALK, EPS8-ALK, GPR113-ALK, HDAC9-ALK, MIPOL1-ALK, PELI1-ALK, SLC39A10-ALK, VKORC1L1-ALK, ALK-SORBS1, ALK-SPINK5, GCC2-ALK, HIP1-ALK, KANK1-ALK, PLEKHA7-ALK, KLC1-ALK, TFG-ALK, TPM3-ALK, or PPFIBP1-ALK fusion nucleic acid molecule of the disclosure, wherein the order of the genes is in the 5′ to 3′ direction. In some embodiments, provided herein are fusion polypeptides encoded by any of the ALK fusion nucleic acid molecules as described herein and/or in any of Tables 1-6, and/or in the Examples herein. In some embodiments, the ALK fusion polypeptide comprises an ALK kinase domain, or a fragment of an ALK kinase domain having ALK kinase activity. In some embodiments, the ALK fusion polypeptide has ALK kinase activity. In some embodiments, the kinase activity is constitutive. In some embodiments, the ALK fusion polypeptide is oncogenic. In some embodiments, the ALK fusion polypeptide promotes cancer cell survival, angiogenesis, cancer cell proliferation, and any combination thereof.

In some aspects, provided herein are BRAF fusion polypeptides that comprise at least a portion of a BRAF polypeptide and at least a portion of a polypeptide encoded by another gene, e.g., a CCDC88C, COBLL1, CREB3L2, DLC1, GOLGA3, MSI2, TNS3, DOCK4, RAD51, AKAP9, ARMC10, DENND2A, JHDM1D, KIAA1549, MKRN1, NRF1, SLC45A3, SND1, ZC3HAV1, ZNF277, or TRIM24 polypeptide. For example, in some embodiments, provided herein are fusion polypeptides encoded by a CCDC88C-BRAF, COBLL1-BRAF, CREB3L2-BRAF, DLC1-BRAF, GOLGA3-BRAF, MSI2-BRAF, TNS3-BRAF, BRAF-DOCK4, BRAF-RAD51, AKAP9-BRAF, ARMC10-BRAF, DENND2A-BRAF, JHDM1D-BRAF, KIAA1549-BRAF, MKRN1-BRAF, NRF1-BRAF, SLC45A3-BRAF, SND1-BRAF, BRAF-TRIM24, ZC3HAV1-BRAF, or ZNF277-BRAF fusion nucleic acid molecule of the disclosure, wherein the order of the genes is in the 5′ to 3′ direction. In some embodiments, provided herein are fusion polypeptides encoded by any of the BRAF fusion nucleic acid molecules as described herein and/or in any of Tables 1-6, and/or in the Examples herein. In some embodiments, the BRAF fusion polypeptide comprises a BRAF kinase domain, or a fragment of a BRAF kinase domain having BRAF kinase activity. In some embodiments, the BRAF fusion polypeptide has BRAF kinase activity. In some embodiments, the kinase activity is constitutive. In some embodiments, the BRAF fusion polypeptide is oncogenic. In some embodiments, the BRAF fusion polypeptide promotes cancer cell survival, angiogenesis, cancer cell proliferation, and any combination thereof.

In some aspects, provided herein are EGFR fusion polypeptides that comprise at least a portion of an EGFR polypeptide and at least a portion of a polypeptide encoded by another gene, e.g., an ABCB1, PDE7A, EZH2, FLJ45974, or ZNF479 polypeptide. For example, in some embodiments, provided herein are fusion polypeptides encoded by an ABCB1-EGFR, PDE7A-EGFR, EGFR-EZH2, EGFR-FLJ45974, or EGFR-ZNF479 fusion nucleic acid molecule of the disclosure, wherein the order of the genes is in the 5′ to 3′ direction. In some embodiments, provided herein are fusion polypeptides encoded by any of the EGFR fusion nucleic acid molecules as described herein and/or in any of Tables 1 and 3-5, and/or in the Examples herein. In some embodiments, the EGFR fusion polypeptide comprises an EGFR kinase domain, or a fragment of an EGFR kinase domain having EGFR kinase activity. In some embodiments, the EGFR fusion polypeptide has EGFR kinase activity. In some embodiments, the kinase activity is constitutive. In some embodiments, the EGFR fusion polypeptide is oncogenic. In some embodiments, the EGFR fusion polypeptide promotes cancer cell survival, angiogenesis, cancer cell proliferation, and any combination thereof.

In some aspects, provided herein are ERBB2 fusion polypeptides that comprise at least a portion of an ERBB2 polypeptide and at least a portion of a polypeptide encoded by another gene, e.g., an FBXL20, GRB7, MSI2, RANBP10, SEC14L1, WIPF2, PRKCA, or PPP1R1B polypeptide. For example, in some embodiments, provided herein are fusion polypeptides encoded by an FBXL20-ERBB2, GRB7-ERBB2, MSI2-ERBB2, RANBP10-ERBB2, SEC14L1-ERBB2, WIPF2-ERBB2, ERBB2-GRB7, ERBB2-PRKCA, or ERBB2-PPP1R1B fusion nucleic acid molecule of the disclosure, wherein the order of the genes is in the 5′ to 3′ direction. In some embodiments, provided herein are fusion polypeptides encoded by any of the ERBB2 fusion nucleic acid molecules as described herein and/or in any of Tables 1-6, and/or in the Examples herein. In some embodiments, the ERBB2 fusion polypeptide comprises an ERBB2 kinase domain, or a fragment of an ERBB2 kinase domain having ERBB2 kinase activity. In some embodiments, the ERBB2 fusion polypeptide has ERBB2 kinase activity. In some embodiments, the kinase activity is constitutive. In some embodiments, the ERBB2 fusion polypeptide is oncogenic. In some embodiments, the ERBB2 fusion polypeptide promotes cancer cell survival, angiogenesis, cancer cell proliferation, and any combination thereof.

In some aspects, provided herein are FGFR1 fusion polypeptides that comprise at least a portion of an FGFR1 polypeptide and at least a portion of a polypeptide encoded by another gene, e.g., an ADAM32, SLC12A8, ADAM18, BAG4, or TACC1 polypeptide. For example, in some embodiments, provided herein are fusion polypeptides encoded by an FGFR1-ADAM32, FGFR1-SLC12A8, ADAM18-FGFR1, BAG4-FGFR1, or FGFR1-TACC1 fusion nucleic acid molecule of the disclosure, wherein the order of the genes is in the 5′ to 3′ direction. In some embodiments, provided herein are fusion polypeptides encoded by any of the FGFR1 fusion nucleic acid molecules as described herein and/or in any of Tables 1-6, and/or in the Examples herein. In some embodiments, the FGFR1 fusion polypeptide comprises an FGFR1 kinase domain, or a fragment of an FGFR1 kinase domain having FGFR1 kinase activity. In some embodiments, the FGFR1 fusion polypeptide has FGFR1 kinase activity. In some embodiments, the kinase activity is constitutive. In some embodiments, the FGFR1 fusion polypeptide is oncogenic. In some embodiments, the FGFR1 fusion polypeptide promotes cancer cell survival, angiogenesis, cancer cell proliferation, and any combination thereof.

In some aspects, provided herein are FGFR2 fusion polypeptides that comprise at least a portion of an FGFR2 polypeptide and at least a portion of a polypeptide encoded by another gene, e.g., an AARSD1, ARMS2, ATF7, BAIAP2L1, CCAR1, CCSER2, CGNL1, EBF1, FANK1, FOXP1, CAMK2G, FLJ40288, GUCY2D, IQGAP2, PAWR, FLNB, IKZF2, KHDRBS1, MYOZ1, PCDH15, PRKAR1A, PRRC2A, RABGAP1, SCIN, STAU1, STK4, TIFA, TLK1, TRIM54, APIP, ATE1, BICC1, TFEC, GRB2, KIAA1217, KIAA1598, MACF1, MYH9, NRAP, RBM20, SPICE1, TACC2, VTI1A, WAC, WARS, or ZMYM4 polypeptide. For example, in some embodiments, provided herein are fusion polypeptides encoded by an FGFR2-AARSD1, FGFR2-ARMS2, FGFR2-ATF7, FGFR2-BAIAP2L1, FGFR2-CCAR1, FGFR2-CCSER2, FGFR2-CGNL1, FGFR2-EBF1, FGFR2-FANK1, FGFR2-FOXP1, CAMK2G-FGFR2, FLJ40288-FGFR2, GUCY2D-FGFR2, IQGAP2-FGFR2, PAWR-FGFR2, FGFR2-FLNB, FGFR2-IKZF2, FGFR2-KHDRBS1, FGFR2-MYOZ1, FGFR2-PCDH15, FGFR2-PRKAR1A, FGFR2-PRRC2A, FGFR2-RABGAP1, FGFR2-SCIN, FGFR2-STAU1, FGFR2-STK4, FGFR2-TIFA, FGFR2-TLK1, FGFR2-TRIM54, FGFR2-APIP, FGFR2-ATE1, FGFR2-BICC1, TFEC-FGFR2, FGFR2-GRB2, FGFR2-KIAA1217, FGFR2-KIAA1598, FGFR2-MACF1, FGFR2-MYH9, FGFR2-NRAP, FGFR2-RBM20, FGFR2-SPICE1, FGFR2-TACC2, FGFR2-VTI1A, FGFR2-WAC, FGFR2-WARS, or FGFR2-ZMYM4 fusion nucleic acid molecule of the disclosure, wherein the order of the genes is in the 5′ to 3′ direction. In some embodiments, provided herein are fusion polypeptides encoded by any of the FGFR2 fusion nucleic acid molecules as described herein and/or in any of Tables 1-6, and/or in the Examples herein. In some embodiments, the FGFR2 fusion polypeptide comprises an FGFR2 kinase domain, or a fragment of an FGFR2 kinase domain having FGFR2 kinase activity. In some embodiments, the FGFR2 fusion polypeptide has FGFR2 kinase activity. In some embodiments, the kinase activity is constitutive. In some embodiments, the FGFR2 fusion polypeptide is oncogenic. In some embodiments, the FGFR2 fusion polypeptide promotes cancer cell survival, angiogenesis, cancer cell proliferation, and any combination thereof.

In some aspects, provided herein are FGFR3 fusion polypeptides that comprise at least a portion of an FGFR3 polypeptide and at least a portion of a polypeptide encoded by another gene, e.g., a CCT5, CNOT4, TNIP2, IGH, TACC3, ADD1, or WHSC1 polypeptide. For example, in some embodiments, provided herein are fusion polypeptides encoded by an FGFR3-CCT5, FGFR3-CNOT4, FGFR3-TNIP2, FGFR3-ADD1, FGFR3-IGH, FGFR3-TACC3, or FGFR3-WHSC1 fusion nucleic acid molecule of the disclosure, wherein the order of the genes is in the 5′ to 3′ direction. In some embodiments, provided herein are fusion polypeptides encoded by any of the FGFR3 fusion nucleic acid molecules as described herein and/or in any of Tables 1-6, and/or in the Examples herein. In some embodiments, the FGFR3 fusion polypeptide comprises an FGFR3 kinase domain, or a fragment of an FGFR3 kinase domain having FGFR3 kinase activity. In some embodiments, the FGFR3 fusion polypeptide has FGFR3 kinase activity. In some embodiments, the kinase activity is constitutive. In some embodiments, the FGFR3 fusion polypeptide is oncogenic. In some embodiments, the FGFR3 fusion polypeptide promotes cancer cell survival, angiogenesis, cancer cell proliferation, and any combination thereof.

In some aspects, provided herein are MET fusion polypeptides that comprise at least a portion of a MET polypeptide and at least a portion of a polypeptide encoded by another gene, e.g., an LDHA, CNTNAP2, HBP1, SNRNP70, CAPZA2, or ST7 polypeptide. For example, in some embodiments, provided herein are fusion polypeptides encoded by a MET-LDHA, CNTNAP2-MET, HBP1-MET, SNRNP70-MET, MET-CAPZA2, or ST7-MET fusion nucleic acid molecule of the disclosure, wherein the order of the genes is in the 5′ to 3′ direction. In some embodiments, provided herein are fusion polypeptides encoded by any of the MET fusion nucleic acid molecules as described herein and/or in any of Tables 1-6, and/or in the Examples herein. In some embodiments, the MET fusion polypeptide comprises a MET kinase domain, or a fragment of a MET kinase domain having MET kinase activity. In some embodiments, the MET fusion polypeptide has MET kinase activity. In some embodiments, the kinase activity is constitutive. In some embodiments, the MET fusion polypeptide is oncogenic. In some embodiments, the MET fusion polypeptide promotes cancer cell survival, angiogenesis, cancer cell proliferation, and any combination thereof.

In some aspects, provided herein are NTRK1 fusion polypeptides that comprise at least a portion of an NTRK1 polypeptide and at least a portion of a polypeptide encoded by another gene, e.g., an MEF2D polypeptide. For example, in some embodiments, provided herein are fusion polypeptides encoded by an NTRK1-MEF2D fusion nucleic acid molecule of the disclosure, wherein the order of the genes is in the 5′ to 3′ direction. In some embodiments, provided herein are fusion polypeptides encoded by any of the NTRK1 fusion nucleic acid molecules as described herein and/or in any of Tables 2 and 6, and/or in the Examples herein. In some embodiments, the NTRK1 fusion polypeptide comprises an NTRK1 kinase domain, or a fragment of an NTRK1 kinase domain having NTRK1 kinase activity. In some embodiments, the NTRK1 fusion polypeptide has NTRK1 kinase activity. In some embodiments, the kinase activity is constitutive. In some embodiments, the NTRK1 fusion polypeptide is oncogenic. In some embodiments, the NTRK1 fusion polypeptide promotes cancer cell survival, angiogenesis, cancer cell proliferation, and any combination thereof.

In some aspects, provided herein are RAF1 fusion polypeptides that comprise at least a portion of a RAF1 polypeptide and at least a portion of a polypeptide encoded by another gene, e.g., a POC1A, SYN2, TRAK1, or ZFYVE20 polypeptide. For example, in some embodiments, provided herein are fusion polypeptides encoded by a POC1A-RAF1, SYN2-RAF1, ZFYVE20-RAF1, or RAF1-TRAK1 fusion nucleic acid molecule of the disclosure, wherein the order of the genes is in the 5′ to 3′ direction. In some embodiments, provided herein are fusion polypeptides encoded by any of the RAF1 fusion nucleic acid molecules as described herein and/or in any of Tables 1-6, and/or in the Examples herein. In some embodiments, the RAF1 fusion polypeptide comprises a RAF1 kinase domain, or a fragment of a RAF1 kinase domain having RAF1 kinase activity. In some embodiments, the RAF1 fusion polypeptide has RAF1 kinase activity. In some embodiments, the kinase activity is constitutive. In some embodiments, the RAF1 fusion polypeptide is oncogenic. In some embodiments, the RAF1 fusion polypeptide promotes cancer cell survival, angiogenesis, cancer cell proliferation, and any combination thereof.

In some aspects, provided herein are RET fusion polypeptides that comprise at least a portion of a RET polypeptide and at least a portion of a polypeptide encoded by another gene, e.g., a ADCY1, NPY4R, PAWR, ALOX5, ARID5B, DHX32, PDE5A, ZNF365, BAIAP2L1, CSGALNACT2, GPHN, NCOA4, RASGEF1A, KIAA1217, CCDC6, ERC1, KIF5B, TRIM24, or VCL polypeptide. For example, in some embodiments, provided herein are fusion polypeptides encoded by a RET-ADCY1, RET-NPY4R, RET-PAWR, ALOX5-RET, ARID5B-RET, DHX32-RET, PDE5A-RET, ZNF365-RET, BAIAP2L1-RET, RET-CSGALNACT2, RET-GPHN, NCOA4-RET, RET-RASGEF1A, KIAA1217-RET, CCDC6-RET, ERC1-RET, KIF5B-RET, TRIM24-RET, or VCL-RET fusion nucleic acid molecule, wherein the order of the genes is in the 5′ to 3′ direction. In some embodiments, provided herein are fusion polypeptides encoded by any of the RET fusion nucleic acid molecules as described herein and/or in any of Tables 1-6, and/or in the Examples herein. In some embodiments, the RET fusion polypeptide comprises a RET kinase domain, or a fragment of a RET kinase domain having RET kinase activity. In some embodiments, the RET fusion polypeptide has RET kinase activity. In some embodiments, the kinase activity is constitutive. In some embodiments, the RET fusion polypeptide is oncogenic. In some embodiments, the RET fusion polypeptide promotes cancer cell survival, angiogenesis, cancer cell proliferation, and any combination thereof.

In some aspects, provided herein are ROS1 fusion polypeptides that comprise at least a portion of a ROS1 polypeptide and at least a portion of a polypeptide encoded by another gene, e.g., an ABR, ASCC3, ELOVL4, QKI, REV3L, MED23, SLC30A8, SLC38A11, TLN1, SLC26A2, SYNGR1, EZR, GOPC, MYO5C, TPD52L1, or TRPC6 polypeptide. For example, in some embodiments, provided herein are fusion polypeptides encoded by a ROS1-ABR, ROS1-ASCC3, ROS1-ELOVL4, ROS1-QKI, ROS1-REV3L, MED23-ROS1, SLC30A8-ROS1, SLC38A11-ROS1, TLN1-ROS1, ROS1-SLC26A2, ROS1-SYNGR1, ROS1-TRPC6, EZR-ROS1, GOPC-ROS1, MYO5C-ROS1, or ROS1-TPD52L1 fusion nucleic acid molecule of the disclosure, wherein the order of the genes is in the 5′ to 3′ direction. In some embodiments, provided herein are fusion polypeptides encoded by any of the ROS1 fusion nucleic acid molecules as described herein and/or in any of Tables 1-6, and/or in the Examples herein. In some embodiments, the ROS1 fusion polypeptide comprises a ROS1 kinase domain, or a fragment of a ROS1 kinase domain having ROS1 kinase activity. In some embodiments, the ROS1 fusion polypeptide has ROS1 kinase activity. In some embodiments, the kinase activity is constitutive. In some embodiments, the ROS1 fusion polypeptide is oncogenic. In some embodiments, the ROS1 fusion polypeptide promotes cancer cell survival, angiogenesis, cancer cell proliferation, and any combination thereof.

### (iii) Cancers and Methods Related Thereto

Certain aspects of the present disclosure relate to methods for identifying an individual having a cancer who may benefit from a treatment comprising an anti-cancer therapy; selecting a treatment for an individual having a cancer; identifying one or more treatment options for an individual having a cancer; predicting survival of an individual having a cancer; treating or delaying progression of cancer; monitoring, evaluating or screening an individual having a cancer; assessing a fusion nucleic acid molecule or polypeptide in a cancer in an individual; detecting the presence or absence of a cancer in an individual; monitoring progression or recurrence of a cancer in an individual; or identifying a candidate treatment for a cancer in an individual in need thereof.

In some embodiments, of any of the methods provide herein, the methods comprise acquiring knowledge of or detecting in a sample from an individual having a cancer a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein and/or in Tables 1-6, and/or in the Examples herein. In other embodiments, the methods comprise acquiring knowledge of or detecting in a sample from an individual having a cancer a fusion polypeptide of the disclosure, e.g., a fusion polypeptide encoded by an ALK, BRAF, EGFR, ERBB2, FGFR1, FGFR2, FGFR3, MET, NTRK1, RAF1, RET, or ROS1 fusion nucleic acid molecule described herein and/or in Tables 1-6, and/or in the Examples herein.

In some embodiments of any of the methods provided herein, detection of the fusion nucleic acid molecule or polypeptide of the disclosure (e.g., an ALK, BRAF, EGFR, ERBB2, FGFR1, FGFR2, FGFR3, MET, NTRK1, RAF1, RET, or ROS1 fusion nucleic acid molecule described herein and/or in Tables 1-6, and/or in the Examples herein; or a fusion polypeptide encoded by such a fusion nucleic acid molecule) in the sample identifies the individual as one who may benefit from a treatment comprising the anti-cancer therapy, such as an anti-cancer therapy provided herein.

In some embodiments, the methods comprise detecting, in a first sample obtained from the individual at a first time point, the presence or absence of a fusion nucleic acid molecule or polypeptide of the disclosure (e.g., an ALK, BRAF, EGFR, ERBB2, FGFR1, FGFR2, FGFR3, MET, NTRK1, RAF1, RET, or ROS1 fusion nucleic acid molecule described herein and/or in Tables 1-6, and/or in the Examples herein; or a fusion polypeptide encoded by such a fusion nucleic acid molecule). In some embodiments, the methods further comprise detecting, in a second sample obtained from the individual at a second time point after the first time point, the presence or absence of a fusion nucleic acid molecule or polypeptide of the disclosure (e.g., an ALK, BRAF, EGFR, ERBB2, FGFR1, FGFR2, FGFR3, MET, NTRK1, RAF1, RET, or ROS1 fusion nucleic acid molecule described herein and/or in Tables 1-6, and/or in the Examples herein; or a fusion polypeptide encoded by such a fusion nucleic acid molecule). In some embodiments, the methods further comprise providing an assessment of cancer progression or cancer recurrence in the individual based, at least in part, on the presence or absence of the fusion nucleic acid molecule or polypeptide in the first sample and/or in the second sample. In some embodiments, the presence of the fusion nucleic acid molecule or polypeptide in the first sample and/or in the second sample identifies the individual as having increased risk of cancer progression or cancer recurrence. In some embodiments, the methods further comprise selecting a treatment, administering a treatment, adjusting a treatment, adjusting the dose of a treatment, or applying a treatment to the individual based, at least in part, on detecting the presence of the fusion nucleic acid molecule or polypeptide in the first sample and/or in the second sample, wherein the treatment comprises an anti-cancer therapy, such as an anti-cancer therapy provided herein.

In some embodiments, the methods comprise performing DNA sequencing on a sample obtained from the individual to determine a sequencing mutation profile on a group of genes comprising one or more of ALK, BRAF, EGFR, ERBB2, FGFR1, FGFR2, FGFR3, MET, NTRK1, RAF1, RET, or ROS1, or any combination thereof, wherein the sequencing mutation profile identifies the presence or absence of a fusion nucleic acid molecule of the disclosure (e.g., an ALK, BRAF, EGFR, ERBB2, FGFR1, FGFR2, FGFR3, MET, NTRK1, RAF1, RET, or ROS1 fusion nucleic acid molecule described herein and/or in Tables 1-6, and/or in the Examples herein). In some embodiments, the methods further comprise identifying a candidate treatment for a cancer in an individual, based at least in part on the sequencing mutation profile. In some embodiments, the candidate treatment comprises an anti-cancer therapy, such as an anti-cancer therapy provided herein. In some embodiments, the sequencing mutation profile identifies the presence or absence of a fragment of the fusion nucleic acid molecule comprising a breakpoint or fusion junction, e.g., one or more of the corresponding breakpoints described herein. In some embodiments, the presence of the fusion nucleic acid molecule in the sample identifies the individual as one who may benefit from a treatment comprising an anti-cancer therapy, e.g., an anti-cancer therapy provided herein. In some embodiments, the presence of the fusion nucleic acid molecule in the sample predicts the individual to have longer survival when treated with a treatment comprising an anti-cancer therapy (e.g., an anti-cancer therapy provided herein), as compared to survival of an individual whose cancer does not comprise the fusion nucleic acid molecule.

In some embodiments of any of the methods provided herein, the methods further comprise generating a report comprising one or more treatment options identified for the individual based at least in part on detection of the fusion nucleic acid molecule or polypeptide in the sample, wherein the one or more treatment options comprise an anti-cancer therapy, such as an anti-cancer therapy provided herein.

In some embodiments of any of the methods provided herein, responsive to acquisition of knowledge of a fusion nucleic acid molecule or polypeptide of the disclosure (e.g., an ALK, BRAF, EGFR, ERBB2, FGFR1, FGFR2, FGFR3, MET, NTRK1, RAF1, RET, or ROS1 fusion nucleic acid molecule described herein and/or in Tables 1-6, and/or in the Examples herein; or a fusion polypeptide encoded by such a fusion nucleic acid molecule) in a sample from the individual: (i) the individual is classified as a candidate to receive a treatment comprising an anti-cancer therapy, such as an anti-cancer therapy provided herein; and/or (ii) the individual is identified as likely to respond to a treatment that comprises an anti-cancer therapy, such as an anti-cancer therapy provided herein. In some embodiments, responsive to acquisition of knowledge of the fusion nucleic acid molecule or polypeptide in a sample from the individual, the individual is predicted to have longer survival when treated with a treatment comprising an anti-cancer therapy, such as an anti-cancer therapy provided herein, as compared to survival of an individual whose cancer does not comprise or exhibit the fusion nucleic acid molecule or polypeptide. In some embodiments, responsive to acquisition of knowledge of the fusion nucleic acid molecule or polypeptide in a sample from the individual, the individual is predicted to have acquired resistance to a prior anti-cancer therapy administered to the individual, the individual is predicted to respond to an anti-cancer therapy (e.g., an anti-cancer therapy provided herein), and/or the individual is predicted to have poor prognosis, as compared to an individual whose cancer does not comprise the fusion nucleic acid molecule or polypeptide.

In some embodiments, responsive to acquisition of knowledge of a fusion nucleic acid molecule or polypeptide (e.g., an ALK, BRAF, EGFR, ERBB2, FGFR1, FGFR2, FGFR3, MET, NTRK1, RAF1, RET, or ROS1 fusion nucleic acid molecule described herein and/or in Tables 1-6, and/or in the Examples herein; or a fusion polypeptide encoded by such a fusion nucleic acid molecule) in a sample from the individual, the methods comprise administering to the individual an effective amount of a treatment that comprises an anti-cancer therapy, such as an anti-cancer therapy provided herein.

In some embodiments of any of the methods provided herein, the methods further comprise generating a report comprising one or more treatment options identified for the individual based at least in part on knowledge of a fusion nucleic acid molecule or polypeptide (e.g., an ALK, BRAF, EGFR, ERBB2, FGFR1, FGFR2, FGFR3, MET, NTRK1, RAF1, RET, or ROS1 fusion nucleic acid molecule described herein and/or in Tables 1-6, and/or in the Examples herein; or a fusion polypeptide encoded by such a fusion nucleic acid molecule) in a sample from the individual, wherein the one or more treatment options comprise an anti-cancer therapy, such as an anti-cancer therapy provided herein.

In some embodiments, acquiring knowledge of a fusion nucleic acid molecule or polypeptide of the disclosure (e.g., an ALK, BRAF, EGFR, ERBB2, FGFR1, FGFR2, FGFR3, MET, NTRK1, RAF1, RET, or ROS1 fusion nucleic acid molecule described herein and/or in Tables 1-6, and/or in the Examples herein; or a fusion polypeptide encoded by such a fusion nucleic acid molecule) in a sample comprises detecting the fusion nucleic acid molecule or polypeptide in the sample.

In some embodiments of any of the methods provided herein, detecting a fusion nucleic acid molecule of the disclosure comprises detecting a fragment of the fusion nucleic acid molecule comprising a breakpoint or fusion junction, e.g., one or more of the corresponding breakpoints described herein.

In some embodiments of any of the methods provided herein, detecting a fusion polypeptide of the disclosure comprises detecting a portion of the fusion polypeptide that is encoded by a fragment of the fusion nucleic acid molecule that comprises a breakpoint or a fusion junction, e.g., one or more of the corresponding breakpoints described herein.

In some embodiments, the methods further comprise providing an assessment of the fusion nucleic acid molecule or polypeptide of the disclosure (e.g., an ALK, BRAF, EGFR, ERBB2, FGFR1, FGFR2, FGFR3, MET, NTRK1, RAF1, RET, or ROS1 fusion nucleic acid molecule described herein and/or in Tables 1-6, and/or in the Examples herein; or a fusion polypeptide encoded by such a fusion nucleic acid molecule).

In some embodiments of any of the methods provided herein, the anti-cancer therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for cancer comprising the fusion nucleic acid molecule or polypeptide, a treatment for cancer being tested in a clinical trial, a targeted therapy, a treatment being tested in a clinical trial for cancer comprising the fusion nucleic acid molecule or polypeptide, or any combination thereof, e.g., a described in further detail below. In some embodiments, the anti-cancer therapy is a kinase inhibitor, such as a kinase inhibitor described herein or known in the art. In some embodiments, the kinase inhibitor is a multi-kinase inhibitor or an ALK-, BRAF-, EGFR-, ERBB2-, FGFR1-, FGFR2-, FGFR3-, MET-, NTRK1-, RAF1-, RET-, or ROS1-specific inhibitor known in the art or described herein. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy. In some embodiments, the nucleic acid inhibits the expression of a fusion nucleic acid molecule or polypeptide of the disclosure (e.g., an ALK, BRAF, EGFR, ERBB2, FGFR1, FGFR2, FGFR3, MET, NTRK1, RAF1, RET, or ROS1 fusion nucleic acid molecule described herein and/or in Tables 1-6, and/or in the Examples herein; or a fusion polypeptide encoded by such a fusion nucleic acid molecule). In some embodiments, the nucleic acid comprises a double-stranded RNA (dsRNA), a small interfering RNA (siRNA), or a small hairpin RNA (shRNA), e.g., as described herein.

In some embodiments of any of the methods provided herein, the methods further comprise acquiring knowledge of or detecting in a sample from the individual a base substitution, a short insertion/deletion (indel), a copy number alteration, or a genomic rearrangement in one or more genes.

In some embodiments of any of the methods provided herein, the fusion nucleic acid molecule or polypeptide is an ALK fusion nucleic acid molecule or polypeptide of the disclosure, e.g., as described herein and/or as listed in any of Tables 1-6, and/or in the Examples herein. In some embodiments, the methods further comprise acquiring knowledge of or detecting, in a sample from the individual, the presence of a mutation in an EGFR gene. In some embodiments, the EGFR mutation is a deletion of exon 19 of EGFR or a portion thereof. In some embodiments, the EGFR mutation results in an L858R, R748K, T790M, C797S, and/or D761N amino acid substitution in an encoded EGFR polypeptide. In some embodiments, the EGFR mutation is an EGFR gene amplification. In some embodiments, the methods further comprise acquiring knowledge of or detecting, in a sample from the individual, the presence of a mutation in a BRAF gene. In some embodiments, the BRAF mutation is a mutation resulting in a V600E amino acid substitution in an encoded BRAF polypeptide. In some embodiments, the methods further comprise acquiring knowledge of or detecting, in a sample from the individual, the presence of a mutation in an NRAS gene. In some embodiments, the NRAS mutation is a mutation resulting in a Q61H amino acid substitution in an encoded NRAS polypeptide. In some embodiments, the methods further comprise acquiring knowledge of or detecting, in a sample from the individual, the presence of a mutation in a MET gene. In some embodiments, the MET mutation is a MET gene amplification. In some embodiments, the MET mutation is a mutation resulting in a D1228H amino acid substitution in an encoded MET polypeptide. In some embodiments, the methods further comprise acquiring knowledge of or detecting, in a sample from the individual, the presence of a mutation in an NF1 gene. In some embodiments, the NF1 mutation is an NF1 truncation. In some embodiments, the methods further comprise acquiring knowledge of or detecting, in a sample from the individual, the presence of a wild type KRAS gene. In some embodiments, the methods further comprise acquiring knowledge of or detecting, in a sample from the individual, the presence of a mutation in a KRAS gene. In some embodiments, the KRAS mutation results in a G12V and/or A146P amino acid substitution in an encoded KRAS polypeptide. In some embodiments, the methods further comprise acquiring knowledge of or detecting, in a sample from the individual, the presence of a mutation in a MAP2K1 gene. In some embodiments, the MAP2K1 mutation is a mutation resulting in a I103_K104del mutation in an encoded MAP2K1 polypeptide. In some embodiments, the methods further comprise acquiring knowledge of or detecting, in a sample from the individual, the presence of an ALK mutation. In some embodiments, the ALK mutation is an ALK resistance mutation. In some embodiments, the ALK resistance mutation results in a G1269A, G1202R, I1171S, I1171T, L1196M, T1151M, S1206Y, 11171N, D1203N, F1174C, L1152R, F1174L, L1198F, C1156Y, T1151_L1152insT, V1180L, G1202L, and/or S1206A amino acid substitution in an encoded ALK polypeptide, or any combination thereof. In some embodiments, the ALK resistance mutation results in a V1180L, 11171N, L1196M, D1203N, or 11171T amino acid substitution in an encoded ALK polypeptide, or any combination thereof. In some embodiments, the methods further comprise acquiring knowledge of or detecting, in a sample from the individual, the presence of one or more ALK gene mutations that result in a V1180L and I1171N amino acid substitution in an encoded ALK polypeptide. In some embodiments, the methods further comprise acquiring knowledge of or detecting, in a sample from the individual, the presence of a D1203N and I1171T amino acid substitution in an encoded ALK polypeptide. In some embodiments of any of the methods provided herein, the ALK fusion nucleic acid molecule or polypeptide of the disclosure confers resistance of the cancer to an EGFR-targeted anti-cancer therapy, such as a first-, second-, or third-generation EGFR tyrosine kinase inhibitor. In some embodiments, the ALK fusion nucleic acid molecule or polypeptide of the disclosure confers resistance of the cancer to an EGFR-targeted anti-cancer therapy, such as cetuximab, panitumumab, lapatinib, gefitinib, vandetanib, dacomitinib, icotinib, osimertinib (AZD9291), afatanib, olmutinib, EGF816 (nazartinib), avitinib (AC00010), rociletinib (CO-1686), BMS-690514, YH5448, PF-06747775, ASP8273, PF299804, AP26113, necitumumab (e.g., Portrazza®), or erlotinib. In some embodiments, the ALK fusion nucleic acid molecule or polypeptide of the disclosure confers resistance of the cancer to an NF1-targeted anti-cancer therapy. In some embodiments, the fusion nucleic acid molecule or polypeptide confers resistance of the cancer to folinic acid, fluorouracil (5-FU), and oxaliplatin (FOLFOX), capecitabine, lonsurf, ramucirumab, bevacizumab, and/or panitumumab. In some embodiments of any of the methods provided herein, the anti-cancer therapy is an ALK-targeted therapy, e.g., as described herein or known in the art. In some embodiments, the ALK-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for ALK-positive or ALK-rearranged cancer, an ALK-targeted therapy being tested in a clinical trial, a treatment for ALK-positive or ALK-rearranged cancer being tested in a clinical trial, or any combination thereof. In some embodiments, the ALK-targeted therapy is a kinase inhibitor known in the art or described herein. In some embodiments, the ALK-targeted therapy is a tyrosine kinase inhibitor known in the art or described herein. In some embodiments, the ALK-targeted therapy is a multi-kinase inhibitor or an ALK-specific inhibitor known in the art or described herein. In some embodiments, the kinase inhibitor inhibits the kinase activity of an ALK polypeptide. In some embodiments, the ALK-targeted therapy comprises one or more of crizotinib, alectinib, ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, TAE684 (NVP-TAE684), CT-707, WX-0593, alkotinib, SIM1803-1A, PLB1003, SAF-189s, PF03446962, TQ-B3101, APG-2449, X-376, CEP-28122, and GSK1838705A. In some embodiments, the nucleic acid inhibits the expression of the ALK fusion nucleic acid molecule or polypeptide. In some embodiments, the cancer is a non-small cell lung carcinoma. In some embodiments, the cancer is an unknown primary carcinoma. In some embodiments, the cancer was previously treated with erlotinib, afatinib, and/or osimertinib.

In some embodiments of any of the methods provided herein, the fusion nucleic acid molecule or polypeptide is a BRAF fusion nucleic acid molecule or polypeptide of the disclosure, e.g., as described herein and/or as listed in any of Tables 1-6, and/or in the Examples herein. In some embodiments, the methods further comprise acquiring knowledge of or detecting, in a sample from the individual, the presence of an EGFR gene mutation. In some embodiments, the EGFR mutation is an EGFR gene amplification. In some embodiments, the EGFR mutation is a mutation resulting in a V441G, S492R, and/or G465E/R amino acid substitution in an encoded EGFR polypeptide. In some embodiments, the methods further comprise acquiring knowledge of or detecting, in a sample from the individual, the presence of a wild type KRAS gene. In some embodiments, the methods further comprise acquiring knowledge of or detecting, in a sample from the individual, the presence of a mutation in a KRAS gene. In some embodiments, the KRAS mutation results in a G12F, G12V, G12C, G13D and/or Q61H amino acid substitution in an encoded KRAS polypeptide. In some embodiments, the methods further comprise acquiring knowledge of or detecting, in a sample from the individual, the presence of a mutation in an NRAS gene. In some embodiments, the mutation in an NRAS gene results in a G13D and/or Q61K/L amino acid substitution in an encoded NRAS polypeptide. In some embodiments, the methods further comprise acquiring knowledge of or detecting, in a sample from the individual, the presence of a mutation in a MET gene. In some embodiments, the mutation in a MET gene is a MET gene amplification. In some embodiments, the methods further comprise acquiring knowledge of or detecting, in a sample from the individual, the presence of a mutation in a MAP2K1 gene. In some embodiments, the mutation in a MAP2K1 gene results in a Q58del or E102_I103del mutation and/or I111T or K57T amino acid substitution in an encoded MAP2K1 polypeptide. In some embodiments, the methods further comprise acquiring knowledge of or detecting, in a sample from the individual, the presence of a mutation in a MAP2K2 gene. In some embodiments, the mutation in the MAP2K2 gene results in a F57V amino acid substitution in an encoded MAP2K2 polypeptide. In some embodiments, the methods further comprise acquiring knowledge of or detecting, in a sample from the individual, the presence of a mutation in an NF1 gene. In some embodiments, the NF1 gene mutation is a F945fs*9 mutation. In some embodiments, the methods further comprise acquiring knowledge of or detecting, in a sample from the individual, the presence of a mutation in a BRAF gene. In some embodiments, the BRAF gene mutation results in a V600E amino acid substitution in an encoded BRAF polypeptide. In some embodiments, the methods further comprise acquiring knowledge of or detecting, in a sample from the individual, the presence of a mutation in an HRAS gene. In some embodiments, the HRAS gene mutation results in a Q61L amino acid substitution in an encoded HRAS polypeptide. In some embodiments, the methods further comprise acquiring knowledge of or detecting, in a sample from the individual, the presence of an EGFR gene amplification and a wild type KRAS gene or a KRAS gene mutation resulting in a G12F, G12V, G12C, G13D and/or Q61H amino acid substitution in an encoded KRAS polypeptide. In some embodiments, the fusion nucleic acid molecule, and/or the encoded fusion polypeptide, confers resistance of the cancer to an EGFR-targeted anti-cancer therapy, such as a first-, second-, or third-generation EGFR tyrosine kinase inhibitor known in the art or described herein. In some embodiments, the fusion nucleic acid molecule, and/or the encoded fusion polypeptide, confers resistance of the cancer to an EGFR-targeted anti-cancer therapy, such as cetuximab, panitumumab, lapatinib, gefitinib, vandetanib, dacomitinib, icotinib, osimertinib (AZD9291), afatanib, olmutinib, EGF816 (nazartinib), avitinib (AC00010), rociletinib (CO-1686), BMS-690514, YH5448, PF-06747775, ASP8273, PF299804, AP26113, necitumumab (e.g., Portrazza®), or erlotinib. In some embodiments, the fusion nucleic acid molecule or polypeptide, confers resistance of the cancer to 5-FU; folinic acid, 5-FU, and irinotecan (FOLFIRI), e.g., in combination with bevacizumab; bevacizumab; or regorafenib. In some embodiments, the fusion nucleic acid molecule or polypeptide, confers resistance of the cancer to FOLFIRI, e.g., in combination with cetuximab. In some embodiments, the fusion nucleic acid molecule or polypeptide, confers resistance of the cancer to folinic acid, 5-FU, and oxaliplatin (FOLFOX), e.g., in combination with bevacizumab. In some embodiments, the fusion nucleic acid molecule or polypeptide, confers resistance of the cancer to pembrolizumab, e.g., in combination with regorafenib. In some embodiments, the fusion nucleic acid molecule or polypeptide, confers resistance of the cancer to adagrasib and/or cetuximab. In some embodiments, the fusion nucleic acid molecule or polypeptide, confers resistance of the cancer to FOLFOXIRI (fluorouracil, leucovorin, oxaliplatin, and irinotecan), bevacizumab, and/or panitumumab. In some embodiments, the fusion nucleic acid molecule or polypeptide confers resistance of the cancer to adagrasib and/or cetuximab. In some embodiments, the fusion nucleic acid molecule or polypeptide confers resistance of the cancer to folinic acid, fluorouracil (5-FU), and oxaliplatin (FOLFOX); 5-FU; folinic acid, 5-FU, and irinotecan (FOLFIRI); and/or regorafenib. In some embodiments, the fusion nucleic acid molecule or polypeptide confers resistance of the cancer to FOLFIRI in combination with bevacizumab; FOLFIRI in combination with cetuximab; folinic acid, 5-FU, and oxaliplatin (FOLFOX) in combination with bevacizumab; and/or pembrolizumab in combination with regorafenib. In some embodiments, the fusion nucleic acid molecule or polypeptide confers resistance of the cancer to adagrasib or adagrasib, e.g., in combination with cetuximab. In some embodiments of any of the methods provided herein, the anti-cancer therapy is a BRAF-targeted therapy known in the art or described herein. In some embodiments, the BRAF-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for BRAF-rearranged cancer, a BRAF-targeted therapy being tested in a clinical trial, a treatment for BRAF-rearranged cancer being tested in a clinical trial, or any combination thereof. In some embodiments, the BRAF-targeted therapy is a kinase inhibitor known in the art or described herein. In some embodiments, the BRAF-targeted therapy is a serine/threonine kinase inhibitor known in the art or described herein. In some embodiments, the BRAF-targeted therapy is a multi-kinase inhibitor or a BRAF-specific inhibitor known in the art or described herein. In some embodiments, the kinase inhibitor inhibits the kinase activity of a BRAF polypeptide. In some embodiments, the BRAF-targeted therapy comprises one or more of sorafenib, PLX4720, PLX-3603, dabrafenib (GSK2118436), encorafenib (LGX818), GDC-0879, RAF265, XL281, ARQ736, BAY73-4506, vemurafenib (e.g., Zelboraf®), cobimetinib (e.g., Cotellic®), binimetinib (e.g., Mektovi®), regorafenib (e.g., Stivarga®), selumetinib (e.g., Koselugo®), trametinib (e.g., Mekinist®), or BAY 43-9006. In some embodiments, the nucleic acid inhibits the expression of the BRAF fusion nucleic acid molecule or polypeptide. In some embodiments, the cancer is a colorectal cancer. In some embodiments, the cancer was previously treated with folinic acid, fluorouracil (5-FU), and oxaliplatin (FOLFOX); 5-FU; folinic acid, 5-FU, and irinotecan (FOLFIRI); and/or regorafenib. In some embodiments, the cancer was previously treated with 5-FU; folinic acid, 5-FU, and irinotecan (FOLFIRI) in combination with bevacizumab; FOLFIRI in combination with cetuximab; folinic acid, 5-FU, and oxaliplatin (FOLFOX) in combination with bevacizumab; and/or pembrolizumab in combination with regorafenib. In some embodiments, the cancer was previously treated with adagrasib or adagrasib in combination with cetuximab.

In some embodiments of any of the methods provided herein, the fusion nucleic acid molecule or polypeptide is an EGFR fusion nucleic acid molecule or polypeptide of the disclosure, e.g., as described herein and/or as listed in any of Tables 1, 3-5, and/or in the Examples herein. In some embodiments, the methods further comprise acquiring knowledge of or detecting, in a sample from the individual, the presence of a wild type KRAS gene. In some embodiments, the methods further comprise acquiring knowledge of or detecting, in a sample from the individual, the presence of a mutation in a KRAS gene. In some embodiments, the KRAS mutation results in a G12A and/or Q61H amino acid substitution in an encoded KRAS polypeptide. In some embodiments, the methods further comprise acquiring knowledge of or detecting, in a sample from the individual, the presence of a mutation in an NRAS gene. In some embodiments, the mutation in an NRAS gene results in a G12D amino acid substitution in an encoded NRAS polypeptide. In some embodiments, the methods further comprise acquiring knowledge of or detecting, in a sample from the individual, the presence of a mutation in a MAP2K1 gene. In some embodiments, the mutation in a MAP2K1 gene results in a E102_I103del mutation. In some embodiments, the anti-cancer therapy is an EGFR-targeted therapy known in the art or described herein. In some embodiments, the EGFR-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for an EGFR-rearranged cancer, an EGFR-targeted therapy being tested in a clinical trial, a treatment for EGFR-rearranged cancer being tested in a clinical trial, or any combination thereof. In some embodiments, the EGFR-targeted therapy is a kinase inhibitor known in the art or described herein. In some embodiments, the EGFR-targeted therapy is a tyrosine kinase inhibitor known in the art or described herein. In some embodiments, the EGFR-targeted therapy is a multi-kinase inhibitor or an EGFR-specific inhibitor known in the art or described herein. In some embodiments, the kinase inhibitor inhibits the kinase activity of an EGFR polypeptide. In some embodiments, the EGFR-targeted therapy comprises one or more of cetuximab, panitumumab, lapatinib, gefitinib, vandetanib, dacomitinib, icotinib, osimertinib (AZD9291), afatanib, olmutinib, EGF816 (nazartinib), avitinib (AC00010), rociletinib (CO-1686), BMS-690514, YH5448, PF-06747775, ASP8273, PF299804, AP26113, necitumumab (e.g., Portrazza®), or erlotinib. In some embodiments, the nucleic acid inhibits the expression of the EGFR fusion nucleic acid molecule or polypeptide.

In some embodiments of any of the methods provided herein, the fusion nucleic acid molecule or polypeptide is an ERBB2 fusion nucleic acid molecule or polypeptide of the disclosure, e.g., as described herein and/or as listed in any of Tables 1-6, and/or in the Examples herein. In some embodiments, the anti-cancer therapy is an ERBB2-targeted therapy known in the art or described herein. In some embodiments, the ERBB2-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for an ERBB2-rearranged cancer, an ERBB2-targeted therapy being tested in a clinical trial, a treatment for ERBB2-rearranged cancer being tested in a clinical trial, or any combination thereof. In some embodiments, the ERBB2-targeted therapy is a kinase inhibitor known in the art or described herein. In some embodiments, the ERBB2-targeted therapy is a tyrosine kinase inhibitor known in the art or described herein. In some embodiments, the ERBB2-targeted therapy is a multi-kinase inhibitor or an ERBB2-specific inhibitor known in the art or described herein. In some embodiments, the kinase inhibitor inhibits the kinase activity of an ERBB2 polypeptide. In some embodiments, the ERBB2-targeted therapy comprises one or more of afatinib, TAK-285, neratinib, dacomitinib, BMS-690514, BMS-599626, pelitinib, CP-724714, lapatinib, TAK-165, ARRY-380, AZD8931, AV-203, AMG-888, MM-111, MM-121, MM-141, LJM716, REGN1400, MEHD7945A, RG7116, trastuzumab, trastuzumab emtansine (T-DM1), pertuzumab, or APC 8024. In some embodiments, the nucleic acid inhibits the expression of the ERBB2 fusion nucleic acid molecule or polypeptide.

In some embodiments of any of the methods provided herein, the fusion nucleic acid molecule or polypeptide is an FGFR1 fusion nucleic acid molecule or polypeptide of the disclosure, e.g., as described herein and/or as listed in any of Tables 1-6, and/or in the Examples herein. In some embodiments, the anti-cancer therapy is an FGFR1-targeted therapy known in the art or described herein. In some embodiments, the FGFR1-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for an FGFR1-rearranged cancer, an FGFR1-targeted therapy being tested in a clinical trial, a treatment for FGFR1-rearranged cancer being tested in a clinical trial, or any combination thereof. In some embodiments, the FGFR1-targeted therapy is a kinase inhibitor known in the art or described herein. In some embodiments, the FGFR1-targeted therapy is a tyrosine kinase inhibitor known in the art or described herein. In some embodiments, the FGFR1-targeted therapy is a multi-kinase inhibitor or an FGFR1-specific inhibitor known in the art or described herein. In some embodiments, the kinase inhibitor inhibits the kinase activity of an FGFR1 polypeptide. In some embodiments, the FGFR1-targeted therapy comprises one or more of E3810 (lucitanib), AZD4547, Dovitinib (TKI258), Ponatinib, Derazantinib (ARQ 087), Nintendanib (BIBF1120), Rogaratinib (BAY 1163877), 3D185, SOMCL-085, brivanib (BMS582664), lenvatinib (E7080), orantinib (TSU-68), PRN1371, XL-228, AZ12908010 (AZ8010), Debio-1347 (CH5183284), FIIN-2, LY2874455, Infigratinib (BGJ398, NVP-BGJ398), Pemigatinib (e.g., Pemazyre®, INCB054828), Erdafitinib (e.g., JNJ-42756493, Balversa®), ASP5878, TAS-120, PRN1371, pazopanib (e.g., Votrient®), regorafenib (e.g., Stivarga®), or PKC412. In some embodiments, the nucleic acid inhibits the expression of the FGFR1 fusion nucleic acid molecule or polypeptide.

In some embodiments of any of the methods provided herein, the fusion nucleic acid molecule or polypeptide is an FGFR2 fusion nucleic acid molecule or polypeptide of the disclosure, e.g., as described herein and/or as listed in any of Tables 1-6, and/or in the Examples herein. In some embodiments, the methods further comprise acquiring knowledge of or detecting, in a sample from the individual, the presence of an EGFR gene mutation. In some embodiments, the EGFR gene mutation results in an L858R, L833V, and/or T790M amino acid substitution in an encoded EGFR polypeptide. In some embodiments, the fusion nucleic acid molecule or polypeptide confers resistance of the cancer to an EGFR-targeted anti-cancer therapy, such as a first-, second-, or third-generation EGFR tyrosine kinase inhibitor. In some embodiments, the fusion nucleic acid molecule or polypeptide confers resistance to an EGFR-targeted anti-cancer therapy, such as cetuximab, panitumumab, lapatinib, gefitinib, vandetanib, dacomitinib, icotinib, osimertinib (AZD9291), afatanib, olmutinib, EGF816 (nazartinib), avitinib (AC00010), rociletinib (CO-1686), BMS-690514, YH5448, PF-06747775, ASP8273, PF299804, AP26113, necitumumab (e.g., Portrazza®), or erlotinib. In some embodiments, the anti-cancer therapy is an FGFR2-targeted therapy known in the art or described herein. In some embodiments, the FGFR2-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for an FGFR2-rearranged cancer, an FGFR2-targeted therapy being tested in a clinical trial, a treatment for FGFR2-rearranged cancer being tested in a clinical trial, or any combination thereof. In some embodiments, the FGFR2-targeted therapy is a kinase inhibitor known in the art or described herein. In some embodiments, the FGFR2-targeted therapy is a tyrosine kinase inhibitor known in the art or described herein. In some embodiments, the FGFR2-targeted therapy is a multi-kinase inhibitor or an FGFR2-specific inhibitor known in the art or described herein. In some embodiments, the kinase inhibitor inhibits the kinase activity of an FGFR2 polypeptide. In some embodiments, the FGFR2-targeted therapy comprises one or more of E3810 (lucitanib), AZD4547, Dovitinib (TKI258), Ponatinib, Derazantinib (ARQ 087), Nintendanib (BIBF1120), Rogaratinib (BAY 1163877), 3D185, SOMCL-085, brivanib (BMS582664), lenvatinib (E7080), orantinib (TSU-68), PRN1371, XL-228, AZ12908010 (AZ8010), Debio-1347 (CH5183284), FIIN-2, LY2874455, Infigratinib (BGJ398, NVP-BGJ398), Pemigatinib (e.g., Pemazyre®, INCB054828), Erdafitinib (e.g., JNJ-42756493, Balversa®), ASP5878, TAS-120, PRN1371, formononetin, R04383596, Ki23057, SU5402, RLY-4008, pazopanib (e.g., Votrient®), regorafenib (e.g., Stivarga®), or PKC412. In some embodiments, the nucleic acid inhibits the expression of the FGFR2 fusion nucleic acid molecule or polypeptide. In some embodiments, the cancer was previously treated with erlotinib.

In some embodiments of any of the methods provided herein, the fusion nucleic acid molecule or polypeptide is an FGFR3 fusion nucleic acid molecule or polypeptide of the disclosure, e.g., as described herein and/or as listed in any of Tables 1-6, and/or in the Examples herein. In some embodiments, the methods further comprise acquiring knowledge of or detecting, in a sample from the individual, the presence of a mutation in an EGFR gene. In some embodiments, the EGFR mutation is a deletion of exon 19 of EGFR or a portion thereof. In some embodiments, the EGFR mutation is an EGFR gene amplification. In some embodiments, the EGFR mutation is a mutation resulting in a T790M, C797G, V441G, G465R, E709K, S492R or L858R amino acid substitution in an encoded EGFR polypeptide, or any combination thereof. In some embodiments, the methods further comprise acquiring knowledge of or detecting, in a sample from the individual, the presence of a mutation in a BRAF gene. In some embodiments, the BRAF mutation results in a V600E amino acid substitution in an encoded BRAF polypeptide. In some embodiments, the methods further comprise acquiring knowledge of or detecting, in a sample from the individual, the presence of a wild type KRAS gene. In some embodiments, the methods further comprise acquiring knowledge of or detecting, in a sample from the individual, the presence of a mutation in a KRAS gene. In some embodiments, the KRAS mutation results in a G12C, G13D, and/or Q61H amino acid substitution in an encoded KRAS polypeptide. In some embodiments, the methods further comprise acquiring knowledge of or detecting, in a sample from the individual, the presence of a mutation in an HRAS gene. In some embodiments, the HRAS mutation results in a Q61L amino acid substitution in an encoded HRAS polypeptide. In some embodiments, the methods further comprise acquiring knowledge of or detecting, in a sample from the individual, the presence of a mutation in an NRAS gene. In some embodiments, the NRAS mutation results in a Q61K amino acid substitution in an encoded NRAS polypeptide. In some embodiments, the methods further comprise acquiring knowledge of or detecting, in a sample from the individual, the presence of a mutation in an ESR1 gene. In some embodiments, the ESR1 mutation results in a Y537N and/or D538G amino acid substitution in an encoded ESR1 polypeptide. In some embodiments, the methods further comprise acquiring knowledge of or detecting, in a sample from the individual, the presence of a mutation in an AKT1 gene. In some embodiments, the AKT1 mutation results in an E17K amino acid substitution in an encoded AKT1 polypeptide. In some embodiments, the sample comprises a deletion of exon 19 of EGFR or a portion thereof. In some embodiments, the sample comprises EGFR gene mutations resulting in an L858R and/or E709K amino acid substitution in an encoded EGFR polypeptide. In some embodiments, the sample comprises an EGFR gene amplification, EGFR gene mutations resulting in a V441G and/or G465R amino acid substitution in an encoded EGFR polypeptide, and a KRAS gene mutation resulting in a Q61H amino acid substitution in an encoded KRAS polypeptide. In some embodiments, the sample comprises an EGFR gene amplification, EGFR gene mutations resulting in a V441G and/or G465R amino acid substitution in an encoded EGFR polypeptide, and a wild type KRAS gene. In some embodiments, the methods further comprise acquiring knowledge of or detecting, in a sample from the individual, the presence of a mutation in a MAP2K1 gene. In some embodiments, the MAP2K1 mutation results in a E102_I103del mutation and/or a K57T amino acid substitution in an encoded MAP2K1 polypeptide. In some embodiments, the methods further comprise acquiring knowledge of or detecting, in a sample from the individual, the presence of an SNRNP70-MET gene fusion. In some embodiments, the sample comprises ESR1 gene mutations resulting in a Y537N and/or D538G amino acid substitution in an encoded ESR1 polypeptide, and AKT1 gene mutations resulting in an E17K amino acid substitution in an encoded AKT1 polypeptide. In some embodiments, the sample comprises a deletion of exon 19 of EGFR or a portion thereof, an EGFR gene mutation resulting in a T790M and/or C797G amino acid substitution in an encoded EGFR polypeptide, and a BRAF gene mutation resulting in a V600E amino acid substitution in an encoded BRAF polypeptide. In some embodiments, the fusion nucleic acid molecule or polypeptide confers resistance of the cancer to hormonal anti-cancer therapy. In some embodiments, the fusion nucleic acid molecule or polypeptide confers resistance of the cancer to everolimus, denosumab, and/or fulvestrant. In some embodiments, the fusion nucleic acid molecule or polypeptide confers resistance of the cancer to adagrasib and/or cetuximab. In some embodiments, the fusion nucleic acid molecule or polypeptide confers resistance of the cancer to an EGFR-targeted anti-cancer therapy, such as a first-, second-, or third-generation EGFR tyrosine kinase inhibitor. In some embodiments, the fusion nucleic acid molecule or polypeptide, confers resistance of the cancer to an EGFR-targeted anti-cancer therapy, such as cetuximab, panitumumab, lapatinib, gefitinib, vandetanib, dacomitinib, icotinib, osimertinib (AZD9291), afatanib, olmutinib, EGF816 (nazartinib), avitinib (AC00010), rociletinib (CO-1686), BMS-690514, YH5448, PF-06747775, ASP8273, PF299804, AP26113, necitumumab (e.g., Portrazza®), or erlotinib. In some embodiments, the fusion nucleic acid molecule or polypeptide, confers resistance of the cancer to 5-FU; folinic acid, 5-FU, and irinotecan (FOLFIRI), e.g., in combination with bevacizumab; bevacizumab; or regorafenib. In some embodiments, the fusion nucleic acid molecule or polypeptide, confers resistance of the cancer to FOLFIRI, e.g., in combination with cetuximab. In some embodiments, the fusion nucleic acid molecule or polypeptide, confers resistance of the cancer to folinic acid, 5-FU, and oxaliplatin (FOLFOX), e.g., in combination with bevacizumab. In some embodiments, the fusion nucleic acid molecule or polypeptide, confers resistance of the cancer to pembrolizumab, e.g., in combination with regorafenib. In some embodiments, the fusion nucleic acid molecule or polypeptide, confers resistance of the cancer to adagrasib and/or cetuximab. In some embodiments, the fusion nucleic acid molecule or polypeptide, confers resistance of the cancer to FOLFOXIRI (fluorouracil, leucovorin, oxaliplatin, and irinotecan), bevacizumab, and/or panitumumab. In some embodiments, the anti-cancer therapy is an FGFR3-targeted therapy known in the art or described herein. In some embodiments, the FGFR3-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for an FGFR3-rearranged cancer, an FGFR3-targeted therapy being tested in a clinical trial, a treatment for FGFR3-rearranged cancer being tested in a clinical trial, or any combination thereof. In some embodiments, the FGFR3-targeted therapy is a kinase inhibitor known in the art or described herein. In some embodiments, the FGFR3-targeted therapy is a tyrosine kinase inhibitor known in the art or described herein. In some embodiments, the FGFR3-targeted therapy is a multi-kinase inhibitor or an FGFR3-specific inhibitor known in the art or described herein. In some embodiments, the kinase inhibitor inhibits the kinase activity of an FGFR3 polypeptide. In some embodiments, the FGFR3-targeted therapy comprises one or more of E3810 (lucitanib), AZD4547, Dovitinib (TKI258), Ponatinib, Derazantinib (ARQ 087), Nintendanib (BIBF1120), Rogaratinib (BAY 1163877), 3D185, SOMCL-085, brivanib (BMS582664), lenvatinib (E7080), orantinib (TSU-68), PRN1371, XL-228, AZ12908010 (AZ8010), Debio-1347 (CH5183284), FIIN-2, LY2874455, Infigratinib (BGJ398, NVP-BGJ398), Pemigatinib (e.g., Pemazyre®, INCB054828), Erdafitinib (e.g., JNJ-42756493, Balversa®), ASP5878, TAS-120, PRN1371, PKC412, Vofatamab (B-70), pazopanib (e.g., Votrient®), or MFGR1877S. In some embodiments, the nucleic acid inhibits the expression of the FGFR3 fusion nucleic acid molecule or polypeptide. In some embodiments, the cancer is a non-small cell lung cancer. In some embodiments, the cancer is a colorectal cancer. In some embodiments, the cancer was previously treated with afatinib and/or cetuximab. In some embodiments, the cancer was previously treated with 5-FU; folinic acid, 5-FU, and irinotecan (FOLFIRI) in combination with bevacizumab; FOLFIRI in combination with cetuximab; folinic acid, 5-FU, and oxaliplatin (FOLFOX) in combination with bevacizumab; and/or pembrolizumab in combination with regorafenib. In some embodiments, the cancer was previously treated with adagrasib or adagrasib in combination with cetuximab. In some embodiments, the cancer was previously treated with FOLFOXIRI (fluorouracil, leucovorin, oxaliplatin, and irinotecan), bevacizumab, and/or panitumumab. In some embodiments, the cancer is a breast cancer, e.g., ER+ and/or PR+ breast cancer. In some embodiments, the cancer was previously treated with everolimus, denosumab, and/or fulvestrant. In some embodiments, the cancer is a non-small cell lung cancer. In some embodiments, the cancer was previously treated with osimertinib.

In some embodiments of any of the methods provided herein, the fusion nucleic acid molecule or polypeptide is a MET fusion nucleic acid molecule or polypeptide of the disclosure, e.g., as described herein and/or as listed in any of Tables 1-6, and/or in the Examples herein. In some embodiments, the methods further comprise acquiring knowledge of or detecting, in a sample from the individual, the presence of a mutation in an EGFR gene. In some embodiments, the EGFR mutation is an EGFR gene amplification. In some embodiments, the EGFR mutation is a mutation resulting in a V441G and/or G465R amino acid substitution in an encoded EGFR polypeptide. In some embodiments, the methods further comprise acquiring knowledge of or detecting, in a sample from the individual, the presence of a wild type KRAS gene. In some embodiments, the methods further comprise acquiring knowledge of or detecting, in a sample from the individual, the presence of a mutation in a KRAS gene. In some embodiments, the mutation results in a Q61H amino acid substitution in an encoded KRAS polypeptide. In some embodiments, the methods further comprise acquiring knowledge of or detecting, in a sample from the individual, the presence of an FGFR3-TACC3 gene fusion. In some embodiments, the fusion nucleic acid molecule or fusion polypeptide confers resistance of the cancer to an EGFR-targeted anti-cancer therapy, such as a first-, second-, or third-generation EGFR tyrosine kinase inhibitor. In some embodiments, the fusion nucleic acid molecule or polypeptide, confers resistance of the cancer to an EGFR-targeted anti-cancer therapy, such as cetuximab, panitumumab, lapatinib, gefitinib, vandetanib, dacomitinib, icotinib, osimertinib (AZD9291), afatanib, olmutinib, EGF816 (nazartinib), avitinib (AC00010), rociletinib (CO-1686), BMS-690514, YH5448, PF-06747775, ASP8273, PF299804, AP26113, necitumumab (e.g., Portrazza®), or erlotinib. In some embodiments, the anti-cancer therapy is a MET-targeted therapy known in the art or described herein. In some embodiments, the MET-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for a MET-rearranged cancer, a MET-targeted therapy being tested in a clinical trial, a treatment for MET-rearranged cancer being tested in a clinical trial, or any combination thereof. In some embodiments, the MET-targeted therapy is a kinase inhibitor known in the art or described herein. In some embodiments, the MET-targeted therapy is a tyrosine kinase inhibitor known in the art or described herein. In some embodiments, the MET-targeted therapy is a multi-kinase inhibitor or a MET-specific inhibitor known in the art or described herein. In some embodiments, the kinase inhibitor inhibits the kinase activity of a MET polypeptide. In some embodiments, the MET-targeted therapy comprises PHA-665752, crizotinib, cabozantinib, and/or capmatinib (INC280). In some embodiments, the nucleic acid inhibits the expression of the MET fusion nucleic acid molecule or polypeptide. In some embodiments, the cancer is a colorectal cancer. In some embodiments, the cancer was previously treated with FOLFOXIRI (fluorouracil, leucovorin, oxaliplatin, and irinotecan), bevacizumab, and/or panitumumab.

In some embodiments of any of the methods provided herein, the fusion nucleic acid molecule or polypeptide is an NTRK1 fusion nucleic acid molecule or polypeptide of the disclosure, e.g., as described herein and/or as listed in any of Tables 2 or 6, and/or in the Examples herein. In some embodiments, the anti-cancer therapy is an NTRK1-targeted therapy known in the art or described herein. In some embodiments, the NTRK1-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for an NTRK1-rearranged cancer, an NTRK1-targeted therapy being tested in a clinical trial, a treatment for NTRK1-rearranged cancer being tested in a clinical trial, or any combination thereof. In some embodiments, the NTRK1-targeted therapy is a kinase inhibitor known in the art or described herein. In some embodiments, the NTRK1-targeted therapy is a tyrosine kinase inhibitor known in the art or described herein. In some embodiments, the NTRK1-targeted therapy is a multi-kinase inhibitor or an NTRK1-specific inhibitor known in the art or described herein. In some embodiments, the kinase inhibitor inhibits the kinase activity of an NTRK1 polypeptide. In some embodiments, the NTRK1-targeted therapy comprises one or more of altiratinib (DCC-2701), AG 879 (Tyrphostin AG 879), an anti-TrK antibody, ARRY 954, AR523, AZ-23, AZ623, a benzotriazole, CEP-2563, danusertib (PHA-739358), entrectinib (also known as RXDX-101 or NMS-E628), DS-6051, GNF 5837, GW 441756, indenopyrrolocarboazole 12a, isothiazole 5n, larotrectinib (previously known as LOXO-101 or ARRY-470), lestaurtinib (CEP-701), selitrectinib (LOXO-195), a macrocyclic compound, ONO-5390556, oxindole 3, pegcantratinib (SNA-120), PHA-848125, PLX7486, a pyrazole derivative, a pyrazolo[1; 5a]pyrimidine, a pyridocarbazole, a pyridoquinazolinyl, a pyridotriazole, a pyrrolidinyl thiourea, a pyrrolidinyl urea, a pyrrolo[2; 3-d]pyrimidine, a quinazolinyl, repotrectinib (TPX-0005), Ro 08-2750, a substituted pyrazolo[1; 5a]pyrimidine, sitravatinib (MGCD516), SNA-125, tavilermide, thiazole 20h, F17752, cabozantinib (XL184), merestinib (LY2801653), belizatinib (TSR-011), dovitinib, ONO-7579, and/or VMD-928. In some embodiments, the nucleic acid inhibits the expression of the NTRK1 fusion nucleic acid molecule or polypeptide.

In some embodiments of any of the methods provided herein, the fusion nucleic acid molecule or polypeptide is a RAF1 fusion nucleic acid molecule or polypeptide of the disclosure, e.g., as described herein and/or as listed in any of Tables 1-6, and/or in the Examples herein. In some embodiments, the methods further comprise acquiring knowledge of or detecting, in a sample from the individual, the presence of a mutation in an EGFR gene. In some embodiments, the EGFR mutation is a mutation resulting in a S492R or V441G amino acid substitution in an encoded EGFR polypeptide, or any combination thereof. In some embodiments, the methods further comprise acquiring knowledge of or detecting, in a sample from the individual, the presence of a mutation in a BRAF gene. In some embodiments, the BRAF mutation results in a V600E amino acid substitution in an encoded BRAF polypeptide. In some embodiments, the methods further comprise acquiring knowledge of or detecting, in a sample from the individual, the presence of a wild type KRAS gene. In some embodiments, the methods further comprise acquiring knowledge of or detecting, in a sample from the individual, the presence of a mutation in a KRAS gene. In some embodiments, the KRAS mutation results in a G12C and/or G13D amino acid substitution in an encoded KRAS polypeptide. In some embodiments, the methods further comprise acquiring knowledge of or detecting, in a sample from the individual, the presence of a mutation in an HRAS gene. In some embodiments, the HRAS mutation results in a Q61L amino acid substitution in an encoded HRAS polypeptide. In some embodiments, the methods further comprise acquiring knowledge of or detecting, in a sample from the individual, the presence of a mutation in an NRAS gene. In some embodiments, the NRAS mutation results in a Q61K amino acid substitution in an encoded NRAS polypeptide. In some embodiments, the methods further comprise acquiring knowledge of or detecting, in a sample from the individual, the presence of a mutation in a MAP2K1 gene. In some embodiments, the MAP2K1 mutation results in a E102_I103del mutation and/or a K57T amino acid substitution in an encoded MAP2K1 polypeptide. In some embodiments, the fusion nucleic acid molecule or polypeptide confers resistance of the cancer to adagrasib and/or cetuximab. In some embodiments, the fusion nucleic acid molecule or polypeptide, confers resistance of the cancer to 5-FU; folinic acid, 5-FU, and irinotecan (FOLFIRI), e.g., in combination with bevacizumab; bevacizumab; or regorafenib. In some embodiments, the fusion nucleic acid molecule or polypeptide, confers resistance of the cancer to FOLFIRI, e.g., in combination with cetuximab. In some embodiments, the fusion nucleic acid molecule or polypeptide, confers resistance of the cancer to folinic acid, 5-FU, and oxaliplatin (FOLFOX), e.g., in combination with bevacizumab. In some embodiments, the fusion nucleic acid molecule or polypeptide, confers resistance of the cancer to pembrolizumab, e.g., in combination with regorafenib. In some embodiments, the fusion nucleic acid molecule or polypeptide, confers resistance of the cancer to adagrasib and/or cetuximab. In some embodiments, the fusion nucleic acid molecule or polypeptide, confers resistance of the cancer to FOLFOXIRI (fluorouracil, leucovorin, oxaliplatin, and irinotecan), bevacizumab, and/or panitumumab. In some embodiments, the anti-cancer therapy is a RAF1-targeted therapy known in the art or described herein. In some embodiments, the RAF1-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for a RAF1-rearranged cancer, a RAF1-targeted therapy being tested in a clinical trial, a treatment for RAF1-rearranged cancer being tested in a clinical trial, or any combination thereof. In some embodiments, the RAF1-targeted therapy is a kinase inhibitor known in the art or described herein. In some embodiments, the RAF1-targeted therapy is a serine/threonine kinase inhibitor known in the art or described herein. In some embodiments, the RAF1-targeted therapy is a multi-kinase inhibitor or a RAF1-specific inhibitor known in the art or described herein. In some embodiments, the kinase inhibitor inhibits the kinase activity of a RAF1 polypeptide. In some embodiments, the RAF1-targeted therapy comprises one or more of Sorafenib (BAY49-9006), Binimetinib (e.g., Mektovi®), Cobimetinib (e.g., Cotellic®), Regorafenib (e.g., Stivarga®), Trametinib (e.g., Mekinit®), or RAF265. In some embodiments, the nucleic acid inhibits the expression of the RAF1 fusion nucleic acid molecule or polypeptide. In some embodiments, the cancer is a colorectal cancer. In some embodiments, the cancer was previously treated with 5-FU; folinic acid, 5-FU, and irinotecan (FOLFIRI) in combination with bevacizumab; FOLFIRI in combination with cetuximab; folinic acid, 5-FU, and oxaliplatin (FOLFOX) in combination with bevacizumab; and/or pembrolizumab in combination with regorafenib. In some embodiments, the cancer was previously treated with adagrasib or adagrasib in combination with cetuximab.

In some embodiments of any of the methods provided herein, the fusion nucleic acid molecule or polypeptide is a RET fusion nucleic acid molecule or polypeptide of the disclosure, e.g., as described herein and/or as listed in any of Tables 1-6, and/or in the Examples herein. In some embodiments, the methods further comprise acquiring knowledge of or detecting, in a sample from the individual, the presence of a mutation in an EGFR gene. In some embodiments, the EGFR mutation is a deletion of exon 19 of EGFR or a portion thereof. In some embodiments, the EGFR mutation is a mutation resulting in a T790M and/or L858R amino acid substitution in an encoded EGFR polypeptide. In some embodiments, the methods further comprise acquiring knowledge of or detecting, in a sample from the individual, the presence of a mutation in a PIK3CA gene. In some embodiments, the PIK3CA mutation results in an E542K amino acid substitution in an encoded PIK3CA polypeptide. In some embodiments, the methods further comprise acquiring knowledge of or detecting, in a sample from the individual, the presence of a mutation in a KRAS gene. In some embodiments, the KRAS mutation results in a G12C amino acid substitution in an encoded KRAS polypeptide. In some embodiments, the methods further comprise acquiring knowledge of or detecting, in a sample from the individual, the presence of a mutation in an ESR1 gene. In some embodiments, the ESR1 mutation results in an E380Q amino acid substitution in an encoded ESR1 polypeptide. In some embodiments, the methods further comprise acquiring knowledge of or detecting, in a sample from the individual, the presence of a mutation in a PTEN gene. In some embodiments, the PTEN mutation results in a S59* and/or M134I amino acid substitution in an encoded PTEN polypeptide. In some embodiments, the methods further comprise acquiring knowledge of or detecting, in a sample from the individual, the presence of a deletion of exon 19 of EGFR or a portion thereof, and an EGFR gene mutation resulting in a T790M amino acid substitution in an encoded EGFR polypeptide. In some embodiments, the fusion nucleic acid molecule or polypeptide, confers resistance of the cancer to an EGFR-targeted anti-cancer therapy, such as a first-, second-, or third-generation EGFR tyrosine kinase inhibitor known in the art or described herein. In some embodiments, the fusion nucleic acid molecule or polypeptide, confers resistance of the cancer to an EGFR-targeted anti-cancer therapy such as cetuximab, panitumumab, lapatinib, gefitinib, vandetanib, dacomitinib, icotinib, osimertinib (AZD9291), afatanib, olmutinib, EGF816 (nazartinib), avitinib (AC00010), rociletinib (CO-1686), BMS-690514, YH5448, PF-06747775, ASP8273, PF299804, AP26113, necitumumab (e.g., Portrazza®), or erlotinib. In some embodiments, the methods further comprise acquiring knowledge of or detecting, in a sample from the individual, the presence of a PIK3CA gene mutation resulting in an E542K amino acid substitution in an encoded PIK3CA polypeptide, an ESR1 gene mutation resulting in a E380Q amino acid substitution in an encoded ESR1 polypeptide, a KRAS gene mutation resulting in a G12C amino acid substitution in an encoded KRAS polypeptide, and a PTEN gene mutation resulting in a S59* and/or M134I amino acid substitution in an encoded PTEN polypeptide. In some embodiments, the fusion nucleic acid molecule or polypeptide confers resistance of the cancer to a PI3K-targeted therapy. In some embodiments, the anti-cancer therapy is a RET-targeted therapy known in the art or described herein. In some embodiments, the RET-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for a RET-rearranged cancer, a RET-targeted therapy being tested in a clinical trial, a treatment for RET-rearranged cancer being tested in a clinical trial, or any combination thereof. In some embodiments, the RET-targeted therapy is a kinase inhibitor known in the art or described herein. In some embodiments, the RET-targeted therapy is a tyrosine kinase inhibitor known in the art or described herein. In some embodiments, the RET-targeted therapy is a multi-kinase inhibitor or a RET-specific inhibitor known in the art or described herein. In some embodiments, the kinase inhibitor inhibits the kinase activity of a RET polypeptide. In some embodiments, the RET-targeted therapy comprises one or more of Selpercatinib (e.g., Retevmo®), Pralsetinib (e.g., Gavreto®), Alectinib (e.g., Alecensa®), Cabozantinib (e.g., Cabometyx®), Lenvatinib (e.g., Lenvima®), Ponatinib (e.g., Iclusig®), Regorafenib (e.g., Stivarga®), Sorafenib (e.g., Nexavar®), Sunitinib (e.g., Sutent®), or Vandetanib (e.g., Caprelsa®). In some embodiments, the nucleic acid inhibits the expression of the RET fusion nucleic acid molecule or polypeptide. In some embodiments, the cancer was previously treated with osimertinib. In some embodiments, the cancer is a breast cancer.

In some embodiments of any of the methods provided herein, the fusion nucleic acid molecule or polypeptide is a ROS1 fusion nucleic acid molecule or polypeptide of the disclosure, e.g., as described herein and/or as listed in any of Tables 1-6, and/or in the Examples herein. In some embodiments, the methods further comprise acquiring knowledge of or detecting, in a sample from the individual, the presence of a PIK3CA gene mutation. In some embodiments, the PIK3CA mutation results in an E545K amino acid substitution in an encoded PIK3CA polypeptide. In some embodiments, the fusion nucleic acid molecule or polypeptide confers resistance of the cancer to a PI3K-targeted therapy. In some embodiments, the anti-cancer therapy is a ROS1-targeted therapy known in the art or described herein. In some embodiments, the ROS1-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for a ROS1-rearranged cancer, a ROS1-targeted therapy being tested in a clinical trial, a treatment for ROS1-rearranged cancer being tested in a clinical trial, or any combination thereof. In some embodiments, the ROS1-targeted therapy is a kinase inhibitor known in the art or described herein. In some embodiments, the ROS1-targeted therapy is a tyrosine kinase inhibitor known in the art or described herein. In some embodiments, the ROS1-targeted therapy is a multi-kinase inhibitor or a ROS1-specific inhibitor known in the art or described herein. In some embodiments, the kinase inhibitor inhibits the kinase activity of a ROS1 polypeptide. In some embodiments, the ROS1-targeted therapy comprises one or more of crizotinib (e.g., Xalkori®), lorlatinib (e.g., Lorbrena®), TQ-B3139, repotrectinib (TPX-0005), brigatinib (e.g., Alunbrig®), cabozantinib (e.g., Cabometyx®), ceritinib (e.g., Zykadia®), or entrectinib. In some embodiments, the nucleic acid inhibits the expression of the ROS1 fusion nucleic acid molecule or polypeptide.

In some embodiments of any of the methods provided herein, the treatment or the one or more treatment options further comprise an additional anti-cancer therapy. In some embodiments of any of the methods provided herein, the treatment or the one or more treatment options further comprise administering an additional anti-cancer therapy to the individual. In some embodiments, the additional anti-cancer therapy is any anti-cancer therapy known in the art or described herein. In some embodiments, the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof.

In some embodiments, the individual has been previously treated, or is being treated, for cancer with a treatment for cancer, e.g., an anti-cancer therapy described herein or any other anti-cancer therapy or treatment known in the art. In some embodiments, the fusion nucleic acid molecule, and/or the fusion polypeptide, confers resistance of the cancer to the treatment for cancer.

In some embodiments of any of the methods provided herein, the cancer is a carcinoma, a sarcoma, a lymphoma, a leukemia, a myeloma, a germ cell cancer, or a blastoma. In some embodiments, the cancer is a solid tumor. In some embodiments, the cancer is a hematologic malignancy. In some embodiments, the cancer is a B cell cancer, a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In some embodiments, the cancer is appendix adenocarcinoma, bladder adenocarcinoma, bladder urothelial (transitional cell) carcinoma, breast cancer not otherwise specified (NOS), breast carcinoma NOS, breast invasive ductal carcinoma (IDC), breast invasive lobular carcinoma (ILC), cervix squamous cell carcinoma (SCC), colon adenocarcinoma (CRC), esophagus adenocarcinoma, esophagus carcinoma NOS, esophagus squamous cell carcinoma (SCC), eye intraocular melanoma, gallbladder adenocarcinoma, gastroesophageal junction adenocarcinoma, intra-hepatic cholangiocarcinoma, kidney cancer NOS, liver hepatocellular carcinoma (HCC), lung cancer NOS, lung adenocarcinoma, lung large cell carcinoma, lung non-small cell lung carcinoma (NSCLC) NOS, lung small cell undifferentiated carcinoma, lung squamous cell carcinoma (SCC), ovary cancer NOS, pancreas cancer NOS, pancreas ductal adenocarcinoma, pancreatobiliary carcinoma, prostate cancer NOS, prostate acinar adenocarcinoma, prostate ductal adenocarcinoma, rectum adenocarcinoma (CRC), skin melanoma, small intestine adenocarcinoma, soft tissue sarcoma NOS, stomach adenocarcinoma NOS, unknown primary cancer NOS, unknown primary adenocarcinoma, unknown primary carcinoma (CUP) NOS, unknown primary neuroendocrine tumor, unknown primary squamous cell carcinoma (SCC), or uterus endometrial adenocarcinoma NOS.

In some embodiments, the methods further comprise detecting the presence or absence of a cancer in a sample from the individual. In some embodiments, the methods further comprise administering an effective amount of anti-cancer therapy to the individual, e.g., an anti-cancer therapy described herein.

In some embodiments, any of the cancers described herein comprise any of the fusion nucleic acid molecules of the disclosure, e.g., an ALK, BRAF, EGFR, ERBB2, FGFR1, FGFR2, FGFR3, MET, NTRK1, RAF1, RET, or ROS1 fusion nucleic acid molecule described herein (e.g., in Tables 1-6, and/or in the Examples herein). In other embodiments, any of the cancers described herein comprise any of the fusion polypeptides of the disclosure, e.g., a fusion polypeptide encoded by an ALK, BRAF, EGFR, ERBB2, FGFR1, FGFR2, FGFR3, MET, NTRK1, RAF1, RET, or ROS1 fusion nucleic acid molecule described herein (e.g., in Tables 1-6, and/or in the Examples herein).

In some embodiments, the methods provided herein comprise acquiring knowledge of or detecting any of the fusion nucleic acid molecules of the disclosure, e.g., an ALK, BRAF, EGFR, ERBB2, FGFR1, FGFR2, FGFR3, MET, NTRK1, RAF1, RET, or ROS1 fusion nucleic acid molecule described herein (e.g., in Tables 1-6, and/or in the Examples herein), in a sample from an individual having any cancer known in the art, or any of the cancers described herein. In some embodiments, the methods provided herein comprise acquiring knowledge of or detecting any of the fusion polypeptides of the disclosure, e.g., a fusion polypeptide encoded by an ALK, BRAF, EGFR, ERBB2, FGFR1, FGFR2, FGFR3, MET, NTRK1, RAF1, RET, or ROS1 fusion nucleic acid molecule described herein (e.g., in Tables 1-6, and/or in the Examples herein), in a sample from an individual having any cancer known in the art, or any of the cancers described herein.

In some embodiments, a cancer provided in Table 2 comprises a corresponding ALK, BRAF, ERBB2, FGFR1, FGFR2, FGFR3, MET, NTRK1, RAF1, RET, or ROS1 fusion nucleic acid molecule as listed in Table 2, or a fusion polypeptide encoded by the fusion nucleic acid molecule.

In some embodiments, the methods provided herein comprise acquiring knowledge of or detecting an ALK, BRAF, ERBB2, FGFR1, FGFR2, FGFR3, MET, NTRK1, RAF1, RET, or ROS1 fusion nucleic acid molecule listed in Table 2, or a fusion polypeptide encoded by such a fusion nucleic acid molecule, in a sample from an individual having a cancer corresponding to the ALK, BRAF, ERBB2, FGFR1, FGFR2, FGFR3, MET, NTRK1, RAF1, RET, or ROS1 fusion nucleic acid molecule as listed in Table 2.

In some embodiments, a cancer provided in Table 6 comprises a corresponding ALK, BRAF, ERBB2, FGFR1, FGFR2, FGFR3, MET, NTRK1, RAF1, RET, or ROS1 fusion nucleic acid molecule as listed in Table 6, or a fusion polypeptide encoded by the fusion nucleic acid molecule.

In some embodiments, the methods provided herein comprise acquiring knowledge of or detecting an ALK, BRAF, ERBB2, FGFR1, FGFR2, FGFR3, MET, NTRK1, RAF1, RET, or ROS1 fusion nucleic acid molecule listed in Table 6, or a fusion polypeptide encoded by such a fusion nucleic acid molecule, in a sample from an individual having a cancer corresponding to the ALK, BRAF, ERBB2, FGFR1, FGFR2, FGFR3, MET, NTRK1, RAF1, RET, or ROS1 fusion nucleic acid molecule as listed in Table 6.

In some embodiments, a cancer provided in Table 4 comprises a corresponding ALK, BRAF, EGFR, ERBB2, FGFR1, FGFR2, FGFR3, MET, RAF1, RET, or ROS1 fusion nucleic acid molecule as listed in Table 4, or a fusion polypeptide encoded by the fusion nucleic acid molecule.

In some embodiments, the methods provided herein comprise acquiring knowledge of or detecting an ALK, BRAF, EGFR, ERBB2, FGFR1, FGFR2, FGFR3, MET, RAF1, RET, or ROS1 fusion nucleic acid molecule listed in Table 4, or a fusion polypeptide encoded by such fusion nucleic acid molecule, in a sample from an individual having a cancer corresponding to the ALK, BRAF, EGFR, ERBB2, FGFR1, FGFR2, FGFR3, MET, RAF1, RET, or ROS1 fusion nucleic acid molecule as listed in Table 4.

In some embodiments, a cancer provided in Table 5 comprises a corresponding ALK, BRAF, EGFR, ERBB2, FGFR1, FGFR2, FGFR3, MET, RAF1, RET, or ROS1 fusion nucleic acid molecule as listed in Table 5, or a fusion polypeptide encoded by the fusion nucleic acid molecule.

In some embodiments, the methods provided herein comprise acquiring knowledge of or detecting an ALK, BRAF, EGFR, ERBB2, FGFR1, FGFR2, FGFR3, MET, RAF1, RET, or ROS1 fusion nucleic acid molecule listed in Table 5, or a fusion polypeptide encoded by such fusion nucleic acid molecule, in a sample from an individual having a cancer corresponding to the ALK, BRAF, EGFR, ERBB2, FGFR1, FGFR2, FGFR3, MET, RAF1, RET, or ROS1 fusion nucleic acid molecule as listed in Table 5.

In some embodiments of any of the methods provided herein, the sample is a sample described below. In some embodiments, the sample is obtained from the individual or from the cancer. In some embodiments, the methods further comprise obtaining the sample, e.g., from the individual or from the cancer. In some embodiments, the sample comprises a tissue biopsy sample, a liquid biopsy sample, or a normal control. In some embodiments, the sample is from a tumor biopsy, tumor specimen, or circulating tumor cell. In some embodiments, the sample is a liquid biopsy sample and comprises blood, plasma, cerebrospinal fluid, sputum, stool, urine, or saliva. In some embodiments, the sample comprises cells and/or nucleic acids from the cancer. In some embodiments, the sample comprises mRNA, DNA, circulating tumor DNA (ctDNA), cell-free DNA, or cell-free RNA from the cancer. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof. In some embodiments, the fusion nucleic acid molecule or polypeptide is detected in a tissue biopsy sample, in a liquid biopsy sample, or in both a tissue biopsy sample and a liquid biopsy sample, from the individual.

### B. Detection of Fusion Nucleic Acid Molecules and Polypeptides

Certain aspects of the present disclosure relate to detection of a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein, e.g., a patient sample. In some embodiments, the fusion nucleic acid molecule is detected in vitro.

Other aspects of the present disclosure relate to detection of a fusion polypeptide of the disclosure, e.g., a fusion polypeptide encoded by a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein, e.g., a patient sample. In some embodiments, the fusion polypeptide is detected in vitro.

### (i) Detection of Fusion Nucleic Acid Molecules

Methods for detecting a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein, are known in the art. For example, in some embodiments, a fusion nucleic acid molecule is detected by sequencing part or all of a gene involved in the fusion nucleic acid molecule, e.g., an ALK, BRAF, EGFR, ERBB2, FGFR1, FGFR2, FGFR3, MET, NTRK1, RAF1, RET, or ROS1 gene, and/or a corresponding fusion partner gene described herein (e.g., as described in any of Tables 1-6, and/or in the Examples herein), by next-generation or other sequencing of DNA, RNA, or cDNA. In some embodiments, a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein, is detected by PCR amplification of DNA, RNA, or cDNA. In some embodiments, a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein, is detected by in situ hybridization using one or more polynucleotides that hybridize to a locus involved in the fusion nucleic acid molecule, e.g., an ALK, BRAF, EGFR, ERBB2, FGFR1, FGFR2, FGFR3, MET, NTRK1, RAF1, RET, or ROS1 locus, and/or a corresponding fusion partner gene locus described herein (e.g., in Tables 1-6, and/or in the Examples herein), e.g., using fluorescence in situ hybridization (FISH). In some embodiments, a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein, is detected in a cancer or tumor cell, e.g., using tumor tissue, such as from a tumor biopsy or other tumor specimen; in a circulating cancer or tumor cell, e.g., using a liquid biopsy, such as from blood, plasma, cerebrospinal fluid, sputum, stool, urine, or saliva; or in circulating tumor DNA (ctDNA), e.g., using a liquid biopsy, such as from blood, plasma, cerebrospinal fluid, sputum, stool, urine, or saliva.

Exemplary and non-limiting methods for detecting a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein, are provided below.

In some embodiments, a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein, is detected using any suitable method known in the art, such as a nucleic acid hybridization assay, an amplification-based assay (e.g., polymerase chain reaction, PCR), a PCR-RFLP assay, real-time PCR, sequencing (e.g., Sanger sequencing or next-generation sequencing), a screening analysis (e.g., using karyotype methods), fluorescence in situ hybridization (FISH), break away FISH, spectral karyotyping, multiplex-FISH, comparative genomic hybridization, in situ hybridization, single specific primer-polymerase chain reaction (SSP-PCR), high performance liquid chromatography (HPLC), or mass-spectrometric genotyping. Methods of analyzing samples, e.g., to detect a nucleic acid molecule, are described in U.S. Pat. No. 9,340,830 and in WO2012092426A1, which are hereby incorporated by reference in their entirety. In some embodiments, a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein, is detected by sequencing. In some embodiments, the sequencing comprises a massively parallel sequencing (MPS) technique, whole genome sequencing (WGS), whole exome sequencing, targeted sequencing, direct sequencing, or a Sanger sequencing technique. In some embodiments, the massively parallel sequencing (MPS) technique comprises next-generation sequencing (NGS).

In some embodiments, a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein, is detected using an in situ hybridization method, such as a fluorescence in situ hybridization (FISH) method.

In some embodiments, FISH analysis is used to identify the chromosomal rearrangement resulting in a fusion nucleic acid molecule as described herein. In some embodiments, FISH analysis is used to identify an RNA molecule comprising or encoding a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein. Methods for performing FISH are known in the art and can be used in nearly any type of tissue. In FISH analysis, nucleic acid probes which are detectably labeled, e.g. fluorescently labeled, are allowed to bind to specific regions of DNA, e.g., a chromosome, or an RNA, e.g., an mRNA, and then examined, e.g., through a microscope. See, for example, U.S. Pat. No. 5,776,688. DNA or RNA molecules are first fixed onto a slide, the labeled probe is then hybridized to the DNA or RNA molecules, and then visualization is achieved, e.g., using enzyme-linked label-based detection methods known in the art. Generally, the resolution of FISH analysis is on the order of detection of 60 to 100000 nucleotides, e.g., 60 base pairs (bp) up to 100 kilobase pairs of DNA. Nucleic acid probes used in FISH analysis comprise single stranded nucleic acids. Such probes are typically at least about 50 nucleotides in length. In some embodiments, probes comprise about 100 to about 500 nucleotides. Probes that hybridize with centromeric DNA and locus-specific DNA or RNA are available commercially, for example, from Vysis, Inc. (Downers Grove, Ill.), Molecular Probes, Inc. (Eugene, Oreg.) or from Cytocell (Oxfordshire, UK). Alternatively, probes can be made non-commercially from chromosomal or genomic DNA or other sources of nucleic acids through standard techniques. Examples of probes, labeling and hybridization methods are known in the art.

Several variations of FISH methods are known in the art and are suitable for use according to the methods of the disclosure, including single-molecule RNA FISH, Fiber FISH, Q-FISH, Flow-FISH, MA-FISH, break-away FISH, hybrid fusion-FISH, and multi-fluor FISH or mFISH. In some embodiments, “break-away FISH” is used in the methods provided herein. In break-away FISH, at least one probe targeting a fusion junction or breakpoint and at least one probe targeting an individual gene of the fusion, e.g., at one or more exons and or introns of the gene, are utilized. In normal cells (i.e., cells not having a fusion nucleic acid molecule described herein), both probes are observed (or a secondary color is observed due to the close proximity of the two genes of the gene fusion); and in cells having a fusion nucleic acid molecule described herein, only a single gene probe is observed due to the presence of a rearrangement resulting in the fusion nucleic acid molecule.

In some embodiments, a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein, is detected using an array-based method, such as array-based comparative genomic hybridization (CGH) methods. In array-based CGH methods, a first sample of nucleic acids (e.g., from a sample, such as from a tumor, or a tissue or liquid biopsy) is labeled with a first label, while a second sample of nucleic acids (e.g., a control, such as from a healthy cell/tissue) is labeled with a second label. In some embodiments, equal quantities of the two samples are mixed and co-hybridized to a DNA microarray of several thousand evenly spaced cloned DNA fragments or oligonucleotides, which have been spotted in triplicate on the array. After hybridization, digital imaging systems are used to capture and quantify the relative fluorescence intensities of each of the hybridized fluorophores. The resulting ratio of the fluorescence intensities is proportional to the ratio of the copy numbers of DNA sequences in the two samples. In some embodiments, where there are chromosomal deletions or multiplications, differences in the ratio of the signals from the two labels are detected and the ratio provides a measure of the copy number. Array-based CGH can also be performed with single-color labeling. In single color CGH, a control (e.g., control nucleic acid sample, such as from a healthy cell/tissue) is labeled and hybridized to one array and absolute signals are read, and a test sample (e.g., a nucleic acid sample obtained from an individual or from a tumor, or a tissue or liquid biopsy) is labeled and hybridized to a second array (with identical content) and absolute signals are read. Copy number differences are calculated based on absolute signals from the two arrays.

In some embodiments, a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein, is detected using an amplification-based method. As is known in the art, in such amplification-based methods, a sample of nucleic acids, such as a sample obtained from an individual, a tumor or a tissue or liquid biopsy, is used as a template in an amplification reaction (e.g., Polymerase Chain Reaction (PCR)) using one or more oligonucleotides or primers, e.g., such as one or more oligonucleotides or primers provided herein. The presence of a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein, in the sample can be determined based on the presence or absence of an amplification product. Quantitative amplification methods are also known in the art and may be used according to the methods provided herein. Methods of measurement of DNA copy number at microsatellite loci using quantitative PCR analysis are known in the art. The known nucleotide sequence for genes is sufficient to enable one of skill in the art to routinely select primers to amplify any portion of the gene. Fluorogenic quantitative PCR can also be used. In fluorogenic quantitative PCR, quantitation is based on the amount of fluorescence signals, e.g., TaqMan and Sybr green.

Other amplification methods suitable for use according to the methods provided herein include, e.g., ligase chain reaction (LCR), transcription amplification, self-sustained sequence replication, dot PCR, and linker adapter PCR.

In some embodiments, a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein, is detected using a sequencing method. Any method of sequencing known in the art can be used to detect a fusion nucleic acid molecule provided herein. Exemplary sequencing methods that may be used to detect a fusion nucleic acid molecule provided herein include those based on techniques developed by Maxam and Gilbert or Sanger. Automated sequencing procedures may also be used, e.g., including sequencing by mass spectrometry.

In some embodiments, a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein, is detected using hybrid capture-based sequencing (hybrid capture-based NGS), e.g., using adaptor ligation-based libraries. See, e.g., Frampton, G. M. et al. (2013) Nat. Biotech. 31:1023-1031, which is hereby incorporated by reference. In some embodiments, a fusion nucleic acid molecule of the disclosure is detected using next-generation sequencing (NGS). Next-generation sequencing includes any sequencing method that determines the nucleotide sequence of either individual nucleic acid molecules or clonally expanded proxies for individual nucleic acid molecules in a highly parallel fashion (e.g., greater than 10⁵ molecules may be sequenced simultaneously). Next generation sequencing methods suitable for use according to the methods provided herein are known in the art and include, without limitation, massively parallel short-read sequencing, template-based sequencing, pyrosequencing, real-time sequencing comprising imaging the continuous incorporation of dye-labeling nucleotides during DNA synthesis, nanopore sequencing, sequencing by hybridization, nano-transistor array based sequencing, polony sequencing, scanning tunneling microscopy (STM)-based sequencing, or nanowire-molecule sensor based sequencing. See, e.g., Metzker, M. (2010) Nature Biotechnology Reviews 11:31-46, which is hereby incorporated by reference. Exemplary NGS methods and platforms that may be used to detect a fusion nucleic acid molecule provided herein include, without limitation, the HeliScope Gene Sequencing system from Helicos BioSciences (Cambridge, MA., USA), the PacBio RS system from Pacific Biosciences (Menlo Park, CA, USA), massively parallel short-read sequencing such as the Solexa sequencer and other methods and platforms from Illumina Inc. (San Diego, CA, USA), 454 sequencing from 454 LifeSciences (Branford, CT, USA), Ion Torrent sequencing from ThermoFisher (Waltham, MA, USA), or the SOLiD sequencer from Applied Biosystems (Foster City, CA, USA). Additional exemplary methods and platforms that may be used to detect a fusion nucleic acid molecule provided herein include, without limitation, the Genome Sequencer (GS) FLX System from Roche (Basel, CHE), the G.007 polonator system, the Solexa Genome Analyzer, HiSeq 2500, HiSeq3000, HiSeq 4000, and NovaSeq 6000 platforms from Illumina Inc. (San Diego, CA, USA).

In some embodiments of any of the methods provided herein, the methods may comprise one or more of the steps of: (i) obtaining a sample from an individual (e.g., an individual suspected of having or determined to have cancer), (ii) extracting nucleic acid molecules (e.g., a mixture of tumor or cancer nucleic acid molecules and non-tumor or non-cancer nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) amplifying the nucleic acid molecules (e.g., using a polymerase chain reaction (PCR) amplification technique, a non-PCR amplification technique, or an isothermal amplification technique), (v) capturing nucleic acid molecules from the amplified nucleic acid molecules (e.g., by hybridization to one or more bait molecules, where the bait molecules each comprise one or more nucleic acid molecules (e.g., capture nucleic acid molecules) that each comprise a region that is complementary to a region of a captured nucleic acid molecule), (vi) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, and (vii) generating, displaying, transmitting, and/or delivering a report (e.g., an electronic, web-based, or paper report) to the individual (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in a graphical user interface of an online or web-based healthcare portal. In some instances, the report is transmitted via a computer network or peer-to-peer connection.

In some embodiments of any of the methods provided herein, the methods may comprise one or more of the steps of: (a) providing a plurality of nucleic acid molecules obtained from a sample from an individual (e.g., an individual suspected of having or determined to have cancer), wherein the plurality of nucleic acid molecules comprises nucleic acid molecules corresponding to a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein (e.g., in Tables 1-6, and/or in the Examples herein); (b) ligating one or more adapters onto one or more nucleic acid molecules from the plurality of nucleic acid molecules; (c) amplifying the one or more ligated nucleic acid molecules from the plurality of nucleic acid molecules; (d) capturing amplified nucleic acid molecules from the amplified nucleic acid molecules; (e) sequencing, by a sequencer, the captured nucleic acid molecules to obtain a plurality of sequence reads that represent the captured nucleic acid molecules, wherein one or more of the plurality of sequence reads correspond to the fusion nucleic acid molecule; (f) analyzing the plurality of sequence reads; and (g) based on the analysis, detecting the presence or absence of the fusion nucleic acid molecule in the sample. In some embodiments, the methods further comprise receiving, at one or more processors, sequence read data for the plurality of sequence reads. In some embodiments, the analyzing the plurality of sequence reads comprises identifying, using the one or more processors, the presence or absence of sequence reads corresponding to the fusion nucleic acid molecule. In some embodiments, the amplified nucleic acid molecules are captured by hybridization with one or more bait molecules.

In some embodiments of any of the methods provided herein, the methods may comprise one or more of the steps of: (a) providing a sample from an individual (e.g., an individual suspected of having or determined to have cancer), wherein the sample comprises a plurality of nucleic acid molecules; (b) preparing a nucleic acid sequencing library from the plurality of nucleic acid molecules in the sample; (c) amplifying said library; (d) selectively enriching for one or more nucleic acid molecules comprising nucleotide sequences corresponding to a fusion nucleic acid molecule of the disclosure (e.g., an ALK, BRAF, EGFR, ERBB2, FGFR1, FGFR2, FGFR3, MET, NTRK1, RAF1, RET, or ROS1 fusion nucleic acid molecule described herein (e.g., in Tables 1-6, and/or in the Examples herein) in said library to produce an enriched sample; (e) sequencing the enriched sample, thereby producing a plurality of sequence reads; (f) analyzing the plurality of sequence reads for the presence of the fusion nucleic acid molecule; (g) detecting, based on the analyzing step, the presence or absence of the fusion nucleic acid molecule in the sample from the individual.

In some embodiments of any of the methods provided herein, the plurality of nucleic acid molecules comprises a mixture of cancer nucleic acid molecules and non-cancer nucleic acid molecules. In some embodiments, the cancer nucleic acid molecules are derived from a tumor portion of a heterogeneous tissue biopsy sample, and the non-cancer nucleic acid molecules are derived from a normal portion of the heterogeneous tissue biopsy sample. In some embodiments, the sample comprises a liquid biopsy sample, and the cancer nucleic acid molecules are derived from a circulating tumor DNA (ctDNA) fraction of the liquid biopsy sample; and the non-cancer nucleic acid molecules are derived from a non-tumor fraction of the liquid biopsy sample or a cell-free DNA (cfDNA) fraction of the liquid biopsy sample.

In some embodiments of any of the methods, the one or more adapters comprise amplification primers, flow cell adaptor sequences, substrate adapter sequences, or sample index sequences. In some embodiments, the selectively enriching comprises: (a) combining one or more bait molecules with the library, thereby hybridizing the one or more bait molecules to one or more nucleic acid molecules comprising nucleotide sequences corresponding to the fusion nucleic acid molecule and producing nucleic acid hybrids; and (b) isolating the nucleic acid hybrids to produce the enriched sample. In some embodiments, the captured nucleic acid molecules are captured from the amplified nucleic acid molecules by hybridization to one or more bait molecules. In some embodiments, the amplifying comprises performing a polymerase chain reaction (PCR) amplification technique, a non-PCR amplification technique, or an isothermal amplification technique. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the sequencing comprises a massively parallel sequencing technique, and the massively parallel sequencing technique comprises next generation sequencing (NGS). In some embodiments, the sequencer comprises a next generation sequencer.

In some embodiments of any of the methods provided herein, the methods further comprise selectively enriching for one or more nucleic acids in the sample comprising nucleotide sequences corresponding to the fusion nucleic acid molecule of the disclosure (e.g., an ALK, BRAF, EGFR, ERBB2, FGFR1, FGFR2, FGFR3, MET, NTRK1, RAF1, RET, or ROS1 fusion nucleic acid molecule described herein (e.g., in Tables 1-6, and/or in the Examples herein). In some embodiments, the selectively enriching produces an enriched sample. In some embodiments, the selectively enriching comprises: (a) combining one or more bait molecules with the sample, thereby hybridizing the one or more bait molecules to one or more nucleic acids in the sample comprising nucleotide sequences corresponding to the fusion nucleic acid molecule and producing nucleic acid hybrids; and (b) isolating the nucleic acid hybrids to produce the enriched sample. In some embodiments, the selectively enriching comprises amplifying the one or more nucleic acids comprising nucleotide sequences corresponding to the fusion nucleic acid molecule using a polymerase chain reaction (PCR) to produce an enriched sample. In some embodiments, the methods further comprise sequencing the enriched sample.

In some embodiments of any of the methods provided herein, the methods further comprise generating a genomic profile for the individual or the sample, based, at least in part, on detecting the presence or absence of the fusion nucleic acid molecule. In some embodiments, the genomic profile for the individual or sample further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the genomic profile further comprises results from a nucleic acid sequencing-based test.

In some embodiments of any of the methods provided herein, the methods further comprise selecting a treatment, administering a treatment, or applying a treatment to the individual based on the generated genomic profile, wherein the treatment comprises an anti-cancer therapy, e.g., as described herein.

In some embodiments of any of the methods provided herein, the methods further comprise generating a report indicating the presence or absence of the fusion nucleic acid molecule in the sample. In some embodiments, the methods further comprise generating, by the one or more processors, a report indicating the presence or absence of the fusion nucleic acid molecule in the sample. In some embodiments, the methods further comprise transmitting the report to a healthcare provider. In some embodiments, the report is transmitted via a computer network or a peer-to-peer connection.

In some embodiments of any of the methods provided herein, the methods further comprise acquiring knowledge of or detecting in a sample from the individual a base substitution, a short insertion/deletion (indel), a copy number alteration, or a genomic rearrangement in one or more genes.

The disclosed methods may be used with any of a variety of samples, e.g., as described in further detail below. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the nucleic acid molecules extracted from a sample may comprise a mixture of tumor or cancer nucleic acid molecules and non-tumor or non-cancer nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor or cancer nucleic acid molecules may be derived from a circulating tumor DNA (ctDNA) fraction of the liquid biopsy sample while the non-tumor or non-cancer nucleic acid molecules may be derived from a non-tumor or non-cancer, cell-free DNA (cfDNA) fraction of the liquid biopsy sample.

In some embodiments of any of the methods provided herein, the method further comprises determining the circulating tumor DNA (ctDNA) fraction of a liquid biopsy sample, e.g., as described herein in Example 1.

### (ii) Detection of Fusion Polypeptides

Also provided herein are methods of detecting a fusion polypeptide of the disclosure, or a fragment thereof, e.g., a fusion polypeptide encoded by a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein (e.g., as described in any of Tables 1-6, and/or in the Examples herein), or a fragment thereof.

A fusion polypeptide provided herein, or a fragment thereof, may be detected or measured, e.g., in a sample obtained from an individual, using any method known in the art, such as using antibodies (e.g., an antibody described herein), mass spectrometry (e.g., tandem mass spectrometry), a reporter assay (e.g., a fluorescence-based assay), immunoblots such as a Western blot, immunoassays such as enzyme-linked immunosorbent assays (ELISA), immunohistochemistry, other immunological assays (e.g., fluid or gel precipitin reactions, immunodiffusion, immunoelectrophoresis, radioimmunoassay (RIA), immunofluorescent assays), and analytic biochemical methods (e.g., electrophoresis, capillary electrophoresis, high performance liquid chromatography (HPLC), thin layer chromatography (TLC), hyperdiffusion chromatography).

In some embodiments, a fusion polypeptide provided herein, or a fragment thereof, can be distinguished from a reference polypeptide, e.g., a non-mutant or wild type protein or polypeptide, with an antibody or antibody fragment that reacts differentially with a mutant protein or polypeptide (e.g., a fusion polypeptide provided herein or a fragment thereof) as compared to a reference protein or polypeptide. In some embodiments, a fusion polypeptide of the disclosure, or a fragment thereof, can be distinguished from a reference polypeptide, e.g., a non-mutant or wild type protein or polypeptide, by reaction with a detection reagent, e.g., a substrate, e.g., a substrate for catalytic activity, e.g., phosphorylation.

In some aspects, methods of detection of a fusion polypeptide of the disclosure (e.g., a fusion polypeptide encoded by a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein, e.g., as described in any of Tables 1-6, and/or in the Examples herein), or a fragment thereof, are provided, comprising contacting a sample, e.g., a sample described herein, comprising a fusion polypeptide described herein, with a detection reagent provided herein (e.g., an antibody of the disclosure), and determining if the fusion polypeptide is present in the sample.

### (iii) Detection Reagents

In some aspects, provided herein are reagents for detecting a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein, or a fragment thereof, e.g., according to the methods of detection provided herein. In some embodiments, a detection reagent provided herein comprises a nucleic acid molecule, e.g., a DNA, RNA, or mixed DNA/RNA molecule, comprising a nucleotide sequence that is complementary to a nucleotide sequence on a target nucleic acid molecule, e.g., a nucleic acid molecule that is or comprises a fusion nucleic acid molecule described herein or a fragment or portion thereof.

In other aspects, provided herein are reagents for detecting a fusion polypeptide of the disclosure, or a fragment thereof, e.g., a fusion polypeptide encoded by a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein (e.g., as described in any of Tables 1-6, and/or in the Examples herein), or a fragment thereof, e.g., according to the methods of detection provided herein. In some embodiments, a detection reagent provided herein comprises an antibody or antibody fragment that specifically binds to a fusion polypeptide of the disclosure, or to a fragment thereof

### Baits

In some embodiments, nucleic acids corresponding to a gene involved in a fusion nucleic acid molecule described herein, e.g., an ALK, BRAF, EGFR, ERBB2, FGFR1, FGFR2, FGFR3, MET, NTRK1, RAF1, RET, or ROS1 gene, and/or a corresponding gene fusion partner as described herein (e.g., in Tables 1-6, and/or in the Examples herein), are captured (e.g., from amplified nucleic acids) by hybridization with a bait molecule. Provided herein are bait molecules suitable for the detection of a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein.

In some embodiments, a bait molecule comprises a capture nucleic acid molecule configured to hybridize to a target nucleic acid molecule comprising a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein, or a fragment or portion thereof. In some embodiments, the capture nucleic acid molecule is configured to hybridize to the ALK, BRAF, EGFR, ERBB2, FGFR1, FGFR2, FGFR3, MET, NTRK1, RAF1, RET, or ROS1 fusion nucleic acid molecule of the target nucleic acid molecule.

In some embodiments, the capture nucleic acid molecule is configured to hybridize to a fragment of a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein. In some embodiments, the fragment comprises (or is) between about 5 and about 25 nucleotides, between about 5 and about 300 nucleotides, between about 100 and about 300 nucleotides, between about 130 and about 230 nucleotides, or between about 150 and about 200 nucleotides. In some embodiments, the fragment comprises (or is) about 100 nucleotides, about 125 nucleotides, about 150 nucleotides, about 175 nucleotides, about 200 nucleotides, about 225 nucleotides, about 250 nucleotides, about 275 nucleotides, or about 300 nucleotides in length. In some embodiments, the fragment comprises a breakpoint or fusion junction of a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein.

In some embodiments, the capture nucleic acid molecule comprises (or is) between about 5 and about 25 nucleotides, between about 5 and about 300 nucleotides, between about 100 and about 300 nucleotides, between about 130 and about 230 nucleotides, or between about 150 and about 200 nucleotides. In some embodiments, the capture nucleic acid molecule comprises (or is) about 100 nucleotides, about 125 nucleotides, about 150 nucleotides, about 175 nucleotides, about 200 nucleotides, about 225 nucleotides, about 250 nucleotides, about 275 nucleotides, or about 300 nucleotides in length.

In some embodiments, the capture nucleic acid molecule is configured to hybridize to a breakpoint of a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein, and may further hybridize to between about 10 and about 100 nucleotides or more, e.g., any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides flanking either side of the breakpoint.

In some embodiments, the capture nucleic acid molecule is configured to hybridize to a nucleotide sequence in an intron or an exon of an ALK, BRAF, EGFR, ERBB2, FGFR1, FGFR2, FGFR3, MET, NTRK1, RAF1, RET, or ROS1 gene, or in a breakpoint joining the introns or exons of an ALK, BRAF, EGFR, ERBB2, FGFR1, FGFR2, FGFR3, MET, NTRK1, RAF1, RET, or ROS1 gene (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides) to an intron or exon of another gene (e.g., a corresponding gene fusion partner as described herein, e.g., in Tables 1-6, and/or in the Examples herein).

In some embodiments, the capture nucleic acid molecule is a DNA, RNA, or a DNA/RNA molecule. In some embodiments, the capture nucleic acid molecule comprises any of between about 50 and about 1000 nucleotides, between about 50 and about 500 nucleotides, between about 100 and about 500 nucleotides, between about 100 and about 300 nucleotides, between about 130 and about 230 nucleotides, or between about 150 and about 200 nucleotides. In some embodiments, the capture nucleic acid molecule comprises any of between about 50 nucleotides and about 100 nucleotides, about 100 nucleotides and about 150 nucleotides, about 150 nucleotides and about 200 nucleotides, about 200 nucleotides and about 250 nucleotides, about 250 nucleotides and about 300 nucleotides, about 300 nucleotides and about 350 nucleotides, about 350 nucleotides and about 400 nucleotides, about 400 nucleotides and about 450 nucleotides, about 450 nucleotides and about 500 nucleotides, about 500 nucleotides and about 550 nucleotides, about 550 nucleotides and about 600 nucleotides, about 600 nucleotides and about 650 nucleotides, about 650 nucleotides and about 700 nucleotides, about 700 nucleotides and about 750 nucleotides, about 750 nucleotides and about 800 nucleotides, about 800 nucleotides and about 850 nucleotides, about 850 nucleotides and about 900 nucleotides, about 900 nucleotides and about 950 nucleotides, or about 950 nucleotides and about 1000 nucleotides. In some embodiments, the capture nucleic acid molecule comprises between about 10 and about 30 nucleotides, between about 50 and about 1000 nucleotides, between about 100 and about 500 nucleotides, between about 100 and about 300 nucleotides, or between about 100 and about 200 nucleotides. In some embodiments, the capture nucleic acid molecule comprises about 150 nucleotides. In some embodiments, the capture nucleic acid molecule is about 150 nucleotides. In some embodiments, the capture nucleic acid molecule comprises about 170 nucleotides. In some embodiments, the capture nucleic acid molecule is about 170 nucleotides.

In some embodiments, a bait provided herein comprises a DNA, RNA, or a DNA/RNA molecule. In some embodiments, a bait provided herein includes a label, a tag or detection reagent. In some embodiments, the label, tag or detection reagent is a radiolabel, a fluorescent label, an enzymatic label, a sequence tag, biotin, or another ligand. In some embodiments, a bait provided herein includes a detection reagent such as a fluorescent marker. In some embodiments, a bait provided herein includes (e.g., is conjugated to) an affinity tag or reagent, e.g., that allows capture and isolation of a hybrid formed by a bait and a nucleic acid molecule hybridized to the bait. In some embodiments, the affinity tag or reagent is an antibody, an antibody fragment, biotin, or any other suitable affinity tag or reagent known in the art. In some embodiments, a bait is suitable for solution phase hybridization.

Baits can be produced and used according to methods known in the art, e.g., as described in WO2012092426A1 and/or or in Frampton et al (2013) Nat Biotechnol, 31:1023-1031, incorporated herein by reference. For example, biotinylated baits (e.g., RNA baits) can be produced by obtaining a pool of synthetic long oligonucleotides, originally synthesized on a microarray, and amplifying the oligonucleotides to produce the bait sequences. In some embodiments, the baits are produced by adding an RNA polymerase promoter sequence at one end of the bait sequences, and synthesizing RNA sequences using RNA polymerase. In one embodiment, libraries of synthetic oligodeoxynucleotides can be obtained from commercial suppliers, such as Agilent Technologies, Inc., and amplified using known nucleic acid amplification methods.

In some embodiments, a bait provided herein is between about 100 nucleotides and about 300 nucleotides. In some embodiments, a bait provided herein is between about 130 nucleotides and about 230 nucleotides. In some embodiments, a bait provided herein is between about 150 nucleotides and about 200 nucleotides. In some embodiments, a bait provided herein comprises a target-specific bait sequence (e.g., a capture nucleic acid molecule described herein) and universal tails on each end. In some embodiments, the target-specific sequence, e.g., a capture nucleic acid molecule described herein, is between about 40 nucleotides and about 300 nucleotides. In some embodiments, the target-specific sequence, e.g., a capture nucleic acid molecule described herein, is between about 100 nucleotides and about 200 nucleotides. In some embodiments, the target-specific sequence, e.g., a capture nucleic acid molecule described herein, is between about 120 nucleotides and about 170 nucleotides. In some embodiments, the target-specific sequence, e.g., a capture nucleic acid molecule described herein, is about 150 nucleotides or about 170 nucleotides. In some embodiments, a bait provided herein comprises an oligonucleotide comprising about 200 nucleotides, of which about 150 nucleotides or about 170 nucleotides are target-specific (e.g., a capture nucleic acid molecule described herein), and the other 50 nucleotides or 30 nucleotides (e.g., 25 or 15 nucleotides on each end of the bait) are universal arbitrary tails, e.g., suitable for PCR amplification.

In some embodiments, a bait provided herein hybridizes to a nucleotide sequence corresponding to an intron or an exon of one gene of a fusion molecule described herein (e.g., an ALK, BRAF, EGFR, ERBB2, FGFR1, FGFR2, FGFR3, MET, NTRK1, RAF1, RET, or ROS1 gene), in an intron or an exon of the other gene of a fusion molecule described herein (e.g., a corresponding gene fusion partner as described herein, e.g., in any of Tables 1-6, and/or in the Examples herein), and/or a breakpoint joining the introns and/or exons.

The baits described herein can be used for selection of exons and short target sequences.

In some embodiments, a bait of the disclosure distinguishes a nucleic acid molecule, e.g., a genomic or transcribed nucleic acid molecule, e.g., a cDNA or RNA, having a breakpoint of a fusion nucleic acid molecule described herein, e.g., as described in any of Tables 3 or 5-6, from a reference nucleotide sequence, e.g., a nucleotide sequence not having the breakpoint.

In some embodiments, the bait hybridizes to a breakpoint of a fusion nucleic acid molecule described herein, e.g., as described in any of Tables 3 or 5-6, and a sequence on either side of the breakpoint (e.g., any of 1, 2, 3, 4, 5, 6, 7, 8, 9, or 10 nucleotides on either side of the breakpoint, or any of between 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more nucleotides on either side of the breakpoint).

### Probes

Also provided herein are probes, e.g., nucleic acid molecules, suitable for the detection of a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein (e.g., in Tables 1-6, and/or in the Examples herein). In some embodiments, a probe provided herein comprises a nucleic acid sequence configured to hybridize to a target nucleic acid molecule that is or comprises a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein, or a fragment or portion thereof. In some embodiments, the probe comprises a nucleic acid sequence configured to hybridize to the fusion nucleic acid molecule of the disclosure, or the fragment or portion thereof, of the target nucleic acid molecule. In some embodiments, the probe comprises a nucleic acid sequence configured to hybridize to a fragment or portion of the fusion nucleic acid molecule of the target nucleic acid molecule. In some embodiments, the fragment or portion comprises between about 5 and about 25 nucleotides, between about 5 and about 300 nucleotides, between about 100 and about 300 nucleotides, between about 130 and about 230 nucleotides, or between about 150 and about 200 nucleotides.

In some embodiments, the probe comprises a nucleotide sequence configured to hybridize to a breakpoint of a fusion nucleic acid molecule of the disclosure, e.g., as described in any of Tables 3 or 5-6, and may be further configured to hybridize to between about 10 and about 100 nucleotides or more, e.g., any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides flanking either side of the breakpoint.

In some embodiments, the probe comprises a nucleotide sequence configured to hybridize to a nucleotide sequence in an intron or an exon of a gene involved in a fusion nucleic acid molecule described herein, e.g., an ALK, BRAF, EGFR, ERBB2, FGFR1, FGFR2, FGFR3, MET, NTRK1, RAF1, RET, or ROS1 gene, or in a breakpoint joining the introns or exons of the gene (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides) to an intron or exon of another gene (e.g., a corresponding gene fusion partner as described herein, e.g., in any of Tables 1-6, and/or in the Examples herein).

In some embodiments, the probe comprises a nucleic acid molecule which is a DNA, RNA, or a DNA/RNA molecule. In some embodiments, the probe comprises a nucleic acid molecule comprising any of between about 10 and about 20 nucleotides, between about 12 and about 20 nucleotides, between about 10 and about 1000 nucleotides, between about 50 and about 500 nucleotides, between about 100 and about 500 nucleotides, between about 100 and about 300 nucleotides, between about 130 and about 230 nucleotides, or between about 150 and about 200 nucleotides. In some embodiments, the probe comprises a nucleic acid molecule comprising any of 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides. In some embodiments, the probe comprises a nucleic acid molecule comprising any of between about 40 nucleotides and about 50 nucleotides, about 50 nucleotides and about 100 nucleotides, about 100 nucleotides and about 150 nucleotides, about 150 nucleotides and about 200 nucleotides, about 200 nucleotides and about 250 nucleotides, about 250 nucleotides and about 300 nucleotides, about 300 nucleotides and about 350 nucleotides, about 350 nucleotides and about 400 nucleotides, about 400 nucleotides and about 450 nucleotides, about 450 nucleotides and about 500 nucleotides, about 500 nucleotides and about 550 nucleotides, about 550 nucleotides and about 600 nucleotides, about 600 nucleotides and about 650 nucleotides, about 650 nucleotides and about 700 nucleotides, about 700 nucleotides and about 750 nucleotides, about 750 nucleotides and about 800 nucleotides, about 800 nucleotides and about 850 nucleotides, about 850 nucleotides and about 900 nucleotides, about 900 nucleotides and about 950 nucleotides, or about 950 nucleotides and about 1000 nucleotides. In some embodiments, the probe comprises a nucleic acid molecule comprising between about 12 and about 20 nucleotides.

In some embodiments, a probe provided herein comprises a DNA, RNA, or a DNA/RNA molecule. In some embodiments, a probe provided herein includes a label or a tag. In some embodiments, the label or tag is a radiolabel (e.g., a radioisotope), a fluorescent label (e.g., a fluorescent compound), an enzymatic label, an enzyme co-factor, a sequence tag, biotin, or another ligand. In some embodiments, a probe provided herein includes a detection reagent such as a fluorescent marker. In some embodiments, a probe provided herein includes (e.g., is conjugated to) an affinity tag, e.g., that allows capture and isolation of a hybrid formed by a probe and a nucleic acid molecule hybridized to the probe. In some embodiments, the affinity tag is an antibody, an antibody fragment, biotin, or any other suitable affinity tag or reagent known in the art. In some embodiments, a probe is suitable for solution phase hybridization.

In some embodiments, probes provided herein may be used according to the methods of detection of fusion nucleic acid molecules provided herein. For example, a probe provided herein may be used for detecting a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein, in a sample, e.g., a sample obtained from an individual. In some embodiments, the probe may be used for identifying cells or tissues that express a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein, e.g., by measuring levels of the fusion nucleic acid molecule. In some embodiments, the probe may be used for detecting levels of a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein, e.g., mRNA levels, in a sample of cells from an individual.

In some embodiments, a probe provided herein specifically hybridizes to a nucleic acid molecule comprising a rearrangement (e.g., a deletion, inversion, insertion, duplication, or other rearrangement) resulting in a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein.

In some embodiments, a probe of the disclosure distinguishes a nucleic acid, e.g., a genomic or transcribed nucleic acid, e.g., a cDNA or RNA, having a breakpoint of a fusion nucleic acid molecule of the disclosure, e.g., as described in any of Tables 3 or 5-6, from a reference nucleotide sequence, e.g., a nucleotide sequence not having the breakpoint.

Also provided herein are isolated pairs of allele-specific probes, wherein, for example, the first probe of the pair specifically hybridizes to a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein, and the second probe of the pair specifically hybridizes to a corresponding wild type sequence (e.g., a wild type ALK, BRAF, EGFR, ERBB2, FGFR1, FGFR2, FGFR3, MET, NTRK1, RAF1, RET, or ROS1 nucleic acid molecule; and/or a wild type nucleic acid molecule corresponding to a gene fusion partner as described herein, e.g., in Tables 1-6, and/or in the Examples herein). Probe pairs can be designed and produced for any of the fusion nucleic acid molecules described herein and are useful in detecting a somatic mutation in a sample. In some embodiments, a first probe of a pair specifically hybridizes to a mutation (e.g., the breakpoint of an alteration, rearrangement, inversion, duplication, deletion, insertion or translocation resulting in a fusion nucleic acid molecule described herein), and a second probe of a pair specifically hybridizes to a sequence upstream or downstream of the mutation.

In some embodiments, one or more probes provided herein are suitable for use in in situ hybridization methods, e.g., as described above, such as FISH.

Chromosomal probes, e.g., for use in the FISH methods described herein, are typically about 50 to about 10⁵ nucleotides in length. Longer probes typically comprise smaller fragments of about 100 to about 500 nucleotides. Probes that hybridize with centromeric DNA and locus-specific DNA are available commercially, for example, from Vysis, Inc. (Downers Grove, Ill.), Molecular Probes, Inc. (Eugene, Oreg.) or from Cytocell (Oxfordshire, UK). Alternatively, probes can be made non-commercially from chromosomal or genomic DNA through standard techniques. For example, sources of DNA that can be used include genomic DNA, cloned DNA sequences, somatic cell hybrids that contain one, or a part of one, chromosome (e.g., human chromosome) along with the normal chromosome complement of the host, and chromosomes purified by flow cytometry or microdissection. The region of interest can be isolated through cloning, or by site-specific amplification via the polymerase chain reaction (PCR). Probes of the disclosure may also hybridize to RNA molecules, e.g., mRNA, such as an RNA that is or comprises a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein.

In some embodiments, probes, such as probes for use in the FISH methods described herein, are used for determining whether a cytogenetic abnormality is present in one or more cells, e.g., in a region of a chromosome or an RNA bound by one or more probes provided herein. The cytogenetic abnormality may be a cytogenetic abnormality that results in a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein. Examples of such cytogenetic abnormalities include, without limitation, deletions (e.g., deletions of entire chromosomes or deletions of fragments of one or more chromosomes), duplications (e.g., of entire chromosomes, or of regions smaller than an entire chromosome), translocations (e.g., non-reciprocal translocations, balanced translocations, reciprocal translocations), intra-chromosomal inversions, point mutations, deletions, gene copy number changes, germ-line mutations, and gene expression level changes.

In some embodiments, probes, such as probes for use in the FISH methods described herein, are labeled such that a chromosomal region or a region on an RNA to which the probes hybridize can be detected. Probes typically are directly labeled with a fluorophore, allowing the probe to be visualized without a secondary detection molecule. Probes can also be labeled by nick translation, random primer labeling or PCR labeling. Labeling may be accomplished using fluorescent (direct)-or haptene (indirect)-labeled nucleotides. Representative, non-limiting examples of labels include: AMCA-6-dUTP, CascadeBlue-4-dUTP, Fluorescein-12-dUTP, Rhodamine-6-dUTP, TexasRed-6-dUTP, Cy3-6-dUTP, Cy5-dUTP, Biotin(BIO)-11-dUTP, Digoxygenin(DIG)-11-dUTP and Dinitrophenyl (DNP)-11-dUTP. Probes can also be indirectly labeled with biotin or digoxygenin, or labeled with radioactive isotopes such as ³²P and ³H, and secondary detection molecules may be used, or further processing may be performed, to visualize the probes. For example, a probe labeled with biotin can be detected by avidin conjugated to a detectable marker, e.g., avidin can be conjugated to an enzymatic marker such as alkaline phosphatase or horseradish peroxidase. Enzymatic markers can be detected in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase. Probes can also be prepared such that a fluorescent or other label is added after hybridization of the probe to its target to detect that the probe hybridized to the target. For example, probes can be used that have antigenic molecules incorporated into the nucleotide sequence. After hybridization, these antigenic molecules are detected, for example, using specific antibodies reactive with the antigenic molecules. Such antibodies can, for example, themselves incorporate a fluorochrome, or can be detected using a second antibody with a bound fluorochrome. For fluorescent probes, e.g., used in FISH techniques, fluorescence can be viewed with a fluorescence microscope equipped with an appropriate filter for each fluorophore, or by using dual or triple band-pass filter sets to observe multiple fluorophores. Alternatively, techniques such as flow cytometry can be used to examine the hybridization pattern of the chromosomal probes.

In some embodiments, the probe hybridizes to a breakpoint of a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein (e.g., as described in any of Tables 3 or 5-6), and a sequence on either side of the breakpoint (e.g., any of 1, 2, 3, 4, 5, 6, 7, 8, 9, or 10 nucleotides on either side of the breakpoint, or any of between 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more nucleotides on either side of the breakpoint).

### Oligonucleotides

In some aspects, provided herein are oligonucleotides, e.g., useful as primers. In some embodiments, an oligonucleotide, e.g., a primer, provided herein comprises a nucleotide sequence configured to hybridize to a target nucleic acid molecule that is or comprises a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein (e.g., in any of Tables 1-6, and/or in the Examples herein), or a fragment or portion thereof. In some embodiments, the oligonucleotide comprises a nucleotide sequence configured to hybridize to the fusion nucleic acid molecule of the target nucleic acid molecule. In some embodiments, the oligonucleotide comprises a nucleotide sequence configured to hybridize to a fragment or portion of the fusion nucleic acid molecule of the target nucleic acid molecule.

In some embodiments, the oligonucleotide, e.g., the primer, comprises a nucleotide sequence configured to hybridize to a breakpoint of a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein (e.g., as described in any of Tables 3 or 5-6), and may be further configured to hybridize to between about 10 and about 12, about 12 and about 15, about 15 and about 17, about 17 and about 20, about 20 and about 25, or about 25 and about 30, or more nucleotides flanking either side of the breakpoint.

In some embodiments, the oligonucleotide, e.g., the primer, comprises a nucleotide sequence configured to hybridize to a nucleotide sequence in an intron or an exon of a gene involved in a fusion nucleic acid mole of the disclosure (e.g., an ALK, BRAF, EGFR, ERBB2, FGFR1, FGFR2, FGFR3, MET, NTRK1, RAF1, RET, or ROS1 gene), to a breakpoint of a fusion nucleic acid molecule described herein (e.g., as described in any of Tables 3 or 5-6), and/or to an intron or exon of another gene (e.g., a corresponding gene fusion partner as described herein, e.g., in any of Tables 1-6, and/or in the Examples herein).

In some embodiments, the oligonucleotide comprises a nucleotide sequence corresponding to a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein (e.g., in any of Tables 1-6, and/or in the Examples herein). In some embodiments, the oligonucleotide comprises a nucleotide sequence corresponding to a fragment or a portion of the fusion nucleic acid molecule. In some embodiments, the fragment or portion comprises between about 10 and about 30 nucleotides, between about 12 and about 20 nucleotides, or between about 12 and about 17 nucleotides. In some embodiments, the oligonucleotide comprises a nucleotide sequence complementary to a fusion nucleic acid molecule provided herein. In some embodiments, the oligonucleotide comprises a nucleotide sequence complementary to a fragment or a portion of the fusion nucleic acid molecule provided herein. In some embodiments, the fragment or portion comprises between about 10 and about 30 nucleotides, between about 12 and about 20 nucleotides, or between about 12 and about 17 nucleotides.

In some embodiments, an oligonucleotide, e.g., a primer, provided herein comprises a nucleotide sequence that is sufficiently complementary to its target nucleotide sequence such that the oligonucleotide specifically hybridizes to a nucleic acid molecule comprising the target nucleotide sequence, e.g., under high stringency conditions. In some embodiments, an oligonucleotide, e.g., a primer, provided herein comprises a nucleotide sequence that is sufficiently complementary to its target nucleotide sequence such that the oligonucleotide specifically hybridizes to a nucleic acid molecule comprising the target nucleotide sequence under conditions that allow a polymerization reaction (e.g., PCR) to occur.

In some embodiments, an oligonucleotide, e.g., a primer, provided herein may be useful for initiating DNA synthesis via PCR (polymerase chain reaction) or a sequencing method. In some embodiments, the oligonucleotide may be used to amplify a nucleic acid molecule that is or comprises a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein (e.g., in any of Tables 1-6, and/or in the Examples herein), or a fragment thereof, e.g., using PCR. In some embodiments, the oligonucleotide may be used to sequence a nucleic acid molecule that is or comprises a fusion nucleic acid molecule provided herein, or a fragment thereof. In some embodiments, the oligonucleotide may be used to amplify a nucleic acid molecule comprising a breakpoint of a fusion nucleic acid molecule described herein (e.g., as described in any of Tables 3 or 5-6), e.g., using PCR. In some embodiments, the oligonucleotide may be used to sequence a nucleic acid molecule comprising a breakpoint of a fusion nucleic acid molecule described herein (e.g., as described in any of Tables 3 or 5-6).

In some embodiments, pairs of oligonucleotides, e.g., pairs of primers, are provided herein, which are configured to hybridize to a nucleic acid molecule that is or comprises a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein (e.g., as described in any of Tables 1-6, and/or in the Examples herein), or a fragment thereof. In some embodiments, a pair of oligonucleotides of the disclosure may be used for directing amplification of the fusion nucleic acid molecule or fragment thereof, e.g., using a PCR reaction. In some embodiments, pairs of oligonucleotides, e.g., pairs of primers, are provided herein, which are configured to hybridize to a nucleic acid molecule comprising a breakpoint of a fusion nucleic acid molecule described herein (e.g., as described in any of Tables 3 or 5-6), e.g., for use in directing amplification of the corresponding fusion nucleic acid molecule or fragment thereof, e.g., using a PCR reaction.

In some embodiments, an oligonucleotide, e.g., a primer, provided herein is a single stranded nucleic acid molecule, e.g., for use in sequencing or amplification methods. In some embodiments, an oligonucleotide provided herein is a double stranded nucleic acid molecule. In some embodiments, a double stranded oligonucleotide is treated, e.g., denatured, to separate its two strands prior to use, e.g., in sequencing or amplification methods. Oligonucleotides provided herein comprise a nucleotide sequence of sufficient length to hybridize to their target, e.g., a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein (e.g., as described in any of Tables 1-6, and/or in the Examples herein), or a fragment thereof, and to prime the synthesis of extension products, e.g., during PCR or sequencing.

In some embodiments, an oligonucleotide, e.g., a primer, provided herein comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more deoxyribonucleotides or ribonucleotides. In some embodiments, an oligonucleotide provided herein comprises at least about 8 deoxyribonucleotides or ribonucleotides. In some embodiments, an oligonucleotide provided herein comprises at least about 10 deoxyribonucleotides or ribonucleotides. In some embodiments, an oligonucleotide provided herein comprises at least about 12 deoxyribonucleotides or ribonucleotides. In some embodiments, an oligonucleotide provided herein comprises at least about 15 deoxyribonucleotides or ribonucleotides. In some embodiments, an oligonucleotide provided herein comprises at least about 20 deoxyribonucleotides or ribonucleotides. In some embodiments, an oligonucleotide provided herein comprises at least about 30 deoxyribonucleotides or ribonucleotides. In some embodiments, an oligonucleotide provided herein comprises between about 10 and about 30 deoxyribonucleotides or ribonucleotides. In some embodiments, an oligonucleotide provided herein comprises between about 10 and about 25 deoxyribonucleotides or ribonucleotides. In some embodiments, an oligonucleotide provided herein comprises between about 10 and about 20 deoxyribonucleotides or ribonucleotides. In some embodiments, an oligonucleotide provided herein comprises between about 10 and about 15 deoxyribonucleotides or ribonucleotides. In some embodiments, an oligonucleotide provided herein comprises between about 12 and about 20 deoxyribonucleotides or ribonucleotides. In some embodiments, an oligonucleotide provided herein comprises between about 17 and about 20 deoxyribonucleotides or ribonucleotides. In some embodiments, the length and nucleotide sequence of an oligonucleotide provided herein is determined according to methods known in the art, e.g., based on factors such as the specific application (e.g., PCR, sequencing library preparation, sequencing), reaction conditions (e.g., buffers, temperature), and the nucleotide composition of the nucleotide sequence of the oligonucleotide or of its target complementary sequence.

In some embodiments, an oligonucleotide, e.g., a primer, of the disclosure distinguishes a nucleic acid, e.g., a genomic or transcribed nucleic acid, e.g., a cDNA or RNA, having a breakpoint of a fusion nucleic acid molecule described herein (e.g., as described in any of Tables 3 or 5-6), from a reference nucleotide sequence, e.g., a nucleotide sequence not having the breakpoint.

In one aspect, provided herein is a primer or primer set for amplifying a nucleic acid molecule comprising a cytogenetic abnormality such as an alteration, rearrangement, chromosomal inversion, deletion, translocation, duplication, or other rearrangement resulting in a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein (e.g., as described in any of Tables 1-6, and/or in the Examples herein). In another aspect, provided herein is a primer or primer set for amplifying a nucleic acid molecule comprising an alteration, rearrangement, chromosomal inversion, insertion, deletion, translocation, duplication or other rearrangement resulting in a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein (e.g., as described in any of Tables 1-6, and/or in the Examples herein). In certain aspects, provided herein are allele-specific oligonucleotides, e.g., primers, wherein a first oligonucleotide of a pair specifically hybridizes to a mutation (e.g., a breakpoint of a fusion nucleic acid molecule described herein, e.g., as described in any of Tables 3 or 5-6), and a second oligonucleotide of a pair specifically hybridizes to a sequence upstream or downstream of the mutation. In certain aspects, provided herein are pairs of oligonucleotides, e.g., primers, wherein a first oligonucleotide of a pair specifically hybridizes to a sequence upstream of a mutation (e.g., a breakpoint of a fusion nucleic acid molecule described herein, e.g., as described in any of Tables 3 or 5-6), and a second oligonucleotide of the pair specifically hybridizes to a sequence downstream of the mutation.

In some embodiments, the oligonucleotide, e.g., the primer, hybridizes to a breakpoint of a fusion nucleic acid molecule described herein, e.g., as described in any of Tables 3 or 5-6, and a sequence on either side of the breakpoint (e.g., any of 1, 2, 3, 4, 5, 6, 7, 8, 9, or 10 nucleotides on either side of the breakpoint, or any of between 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more nucleotides on either side of the breakpoint).

### Antibodies

Provided herein are antibodies or antibody fragments that specifically bind to a fusion polypeptide of the disclosure, or a fragment thereof, e.g., a fusion polypeptide encoded by a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein (e.g., as described in any of Tables 1-6, and/or in the Examples herein), or a fragment thereof.

The antibody may be of any suitable type of antibody, including, but not limited to, a monoclonal antibody, a polyclonal antibody, a multi-specific antibody (e.g., a bispecific antibody), or an antibody fragment, so long as the antibody or antibody fragment exhibits a specific antigen binding activity, e.g., binding to a fusion polypeptide of the disclosure, or a fragment thereof, e.g., a fusion polypeptide encoded by a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein (e.g., as described in any of Tables 1-6, and/or in the Examples herein), or a fragment thereof.

In some embodiments, a fusion polypeptide of the disclosure, or a fragment thereof, e.g., a fusion polypeptide encoded by a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein (e.g., as described in any of Tables 1-6, and/or in the Examples herein), or a fragment thereof, is used as an immunogen to generate one or more antibodies of the disclosure, e.g., using standard techniques for polyclonal and monoclonal antibody preparation. In some embodiments, a fusion polypeptide provided herein, is used to provide antigenic peptide fragments (e.g., comprising any of at least about 8, at least about 10, at least about 15, at least about 20, at least about 30 or more amino acids) for use as immunogens to generate one or more antibodies of the disclosure, e.g., using standard techniques for polyclonal and monoclonal antibody preparation. As is known in the art, an antibody of the disclosure may be prepared by immunizing a suitable (i.e., immunocompetent) subject such as a rabbit, goat, mouse, or other mammal or vertebrate. An appropriate immunogenic preparation can contain, for example, recombinantly-expressed or chemically-synthesized polypeptides, e.g., a fusion polypeptide of the disclosure, or a fragment thereof, e.g., a fusion polypeptide encoded by a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein (e.g., as described in any of Tables 1-6, and/or in the Examples herein), or a fragment thereof. The preparation can further include an adjuvant, such as Freund's complete or incomplete adjuvant, or a similar immunostimulatory agent.

In some embodiments, an antibody provided herein is a polyclonal antibody. Methods of producing polyclonal antibodies are known in the art. In some embodiments, an antibody provided herein is a monoclonal antibody, wherein a population of the antibody molecules contain only one species of an antigen binding site capable of immunoreacting or binding with a particular epitope, e.g., an epitope on a fusion polypeptide provided herein. Methods of preparation of monoclonal antibodies are known in the art, e.g., using standard hybridoma techniques originally described by Kohler and Milstein (1975) Nature 256:495-497, human B cell hybridoma techniques (see Kozbor et al., 1983, Immunol. Today 4:72), EBV-hybridoma techniques (see Cole et al., pp. 77-96 In Monoclonal Antibodies and Cancer Therapy, Alan R. Liss, Inc., 1985), or trioma techniques. The technology for producing hybridomas is well known (see generally Current Protocols in Immunology, Coligan et al. ed., John Wiley & Sons, New York, 1994). A monoclonal antibody of the disclosure may also be identified and isolated by screening a recombinant combinatorial immunoglobulin library (e.g., an antibody phage display library) with the polypeptide of interest, e.g., a fusion polypeptide provided herein or a fragment thereof. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display libraries can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; and Griffiths et al. (1993) EMBO J. 12:725-734. In some embodiments, monoclonal antibodies of the disclosure are recombinant antibodies, such as chimeric and humanized monoclonal antibodies, comprising both human and non-human portions. Such chimeric and/or humanized monoclonal antibodies can be produced by recombinant DNA techniques known in the art, for example, using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Nati. Acad. Sci. USA 84:3439-3443; Liu et al. (1987) J. Immunol. 139:3521-3526; Sun et al. (1987) Proc. P Natl. Acad. Sci. USA 84:214-218; Nishimura et al. (1987) Cancer Res. 47:999-1005; Wood et al. (1985) Nature 314:446-449; Shaw et al. (1988) J. Nati. Cancer Inst. 80:1553-1559; Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060. In some embodiments, a monoclonal antibody of the disclosure is a human monoclonal antibody. In some embodiments, human monoclonal antibodies are prepared using methods known in the art, e.g., using transgenic mice which are incapable of expressing endogenous immunoglobulin heavy and light chains genes, but which can express human heavy and light chain genes. For an overview of this technology for producing human antibodies, see Lonberg and Huszar (1995) Int. Rev. Immunol. 13:65-93. For a detailed discussion of this technology for producing human antibodies and human monoclonal antibodies, and protocols for producing such antibodies, see, e.g., U.S. Pat. Nos. 5,625,126; 5,633,425; 5,569,825; 5,661,016; and 5,545,806.

In some embodiments, the antibody or antibody fragment of the disclosure is an isolated antibody or antibody fragment, which has been separated from a component of its natural environment or a cell culture used to produce the antibody or antibody fragment. In some embodiments, an antibody of the disclosure is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods.

In some embodiments, an antibody of the disclosure can be used to isolate a fusion polypeptide provided herein, or a fragment thereof, by standard techniques, such as affinity chromatography or immunoprecipitation. In some embodiments, an antibody of the disclosure can be used to detect a fusion polypeptide provided herein, or a fragment thereof, e.g., in a tissue sample, cellular lysate, or cell supernatant, in order to evaluate the level and/or pattern of expression of the fusion polypeptide. Detection can be facilitated by coupling the antibody to a detectable substance. Thus, in some embodiments, an antibody of the disclosure is coupled to a detectable substance, such as enzymes, prosthetic groups, fluorescent materials, luminescent materials, bioluminescent materials, and radioactive materials. Non-limiting examples of suitable enzymes include, e.g., horseradish peroxidase, alkaline phosphatase, β-galactosidase, or acetylcholinesterase; examples of suitable prosthetic group complexes include, e.g., streptavidin/biotin and avidin/biotin; examples of suitable fluorescent materials include, e.g., umbelliferone, fluorescein, fluorescein isothiocyanate, rhodamine, dichlorotriazinylamine fluorescein, dansyl chloride or phycoerythrin; an example of a luminescent material includes, but is not limited to, luminol; examples of bioluminescent materials include, e.g., luciferase, luciferin, and aequorin; and examples of suitable radioactive materials include, e.g., ¹²⁵I, ¹³¹I, ³⁵S or ³H.

An antibody or antibody fragment of the disclosure may also be used diagnostically, e.g., to detect and/or monitor protein levels (e.g., protein levels of a fusion polypeptide provided herein) in tissues or body fluids (e.g., in a tumor cell-containing tissue or body fluid), e.g., according to the methods provided herein.

In certain embodiments, an antibody provided herein has a dissociation constant (Kd) of ≤1 μM, ≤100 nM, ≤10 nM, ≤1 nM, ≤0.1 nM, ≤0.01 nM, or ≤0.001 nM (e.g., 10⁻⁸M or less, e.g., from 10⁻⁸ M to 10⁻¹³M, e.g., from 10⁻⁹ M to 10⁻¹³ M). Methods of measuring antibody affinity (e.g., Kd) are known in the art, and include, without limitation, a radiolabeled antigen binding assay (RIA) and a BIACORE® surface plasmon resonance assay. In some embodiments, antibody affinity (e.g., Kd) is determined using the Fab version of an antibody of the disclosure and its antigen (e.g., a fusion polypeptide provided herein). In some embodiments, a RIA is performed with the Fab version of an antibody of the disclosure and its antigen (e.g., a fusion polypeptide provided herein).

In certain embodiments, an antibody provided herein is an antibody fragment. Antibody fragments include, but are not limited to, Fab, Fab′, Fab′-SH, F(ab′)₂, Fv, and single-chain antibody molecule (e.g., scFv) fragments, and other fragments described herein or known in the art.

In certain embodiments, an antibody provided herein is a diabody. Diabodies are antibody fragments with two antigen-binding sites that may be bivalent or bispecific. In certain embodiments, an antibody provided herein is a triabody or a tetrabody.

In certain embodiments, an antibody provided herein is a single-domain antibody. Single-domain antibodies are antibody fragments comprising all or a portion of the heavy chain variable domain or all or a portion of the light chain variable domain of an antibody. In certain embodiments, a single-domain antibody is a human single-domain antibody.

Antibody fragments can be made by various techniques, including but not limited to proteolytic digestion of an intact antibody, as well as production by recombinant host cells (e.g., E. coli or phage), as known in the art and as described herein.

In certain embodiments, an antibody provided herein is a chimeric antibody. In one example, a chimeric antibody comprises a non-human variable region (e.g., a variable region derived from a mouse, rat, hamster, rabbit, or non-human primate, such as a monkey), and a human constant region. In a further example, a chimeric antibody is a “class switched” antibody, in which the class or subclass of the antibody has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof), are derived from a non-human antibody, and framework regions (FRs) (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity. Humanized antibodies and methods of making them are known in the art. Human framework regions that may be used for humanization include but are not limited to: framework regions selected using the “best-fit” method; framework regions derived from the consensus sequence of human antibodies of a particular subgroup of light or heavy chain variable regions; human mature (somatically mutated) framework regions or human germline framework regions; and framework regions derived from screening FR libraries.

In certain embodiments, an antibody provided herein is a human antibody. Human antibodies can be produced using various techniques known in the art. For example, human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or are present extrachromosomally or integrated randomly into the animal's chromosomes. In such transgenic animals, e.g., mice, the endogenous immunoglobulin loci have generally been inactivated. Human variable regions from intact antibodies generated by such animals may be further modified, e.g., by combining with a different human constant region. Human antibodies can also be made by hybridoma-based methods known in the art, e.g., using known human myeloma and mouse-human heteromyeloma cell lines for the production of human monoclonal antibodies. Human antibodies may also be generated by isolating Fv clone variable domain sequences selected from human-derived phage display libraries. Such variable domain sequences may then be combined with a desired human constant domain. Techniques for selecting human antibodies from antibody libraries are known in the art and described herein.

Antibodies of the disclosure may be isolated by screening combinatorial libraries for antibodies with the desired activity or activities. For example, a variety of methods are known in the art for generating phage display libraries and screening such libraries for antibodies possessing the desired binding characteristics. In certain phage display methods, repertoires of VH and VL genes are separately cloned by polymerase chain reaction (PCR) and recombined randomly in phage libraries, which can then be screened for antigen-binding phage. Phage typically display antibody fragments, either as single-chain Fv (scFv) fragments or as Fab fragments. Libraries from immunized sources provide high-affinity antibodies to the immunogen without the requirement of constructing hybridomas. Alternatively, a naive antibody repertoire can be cloned (e.g., from human) to provide a single source of antibodies to a wide range of non-self and also self antigens without any immunization. Naive libraries can also be made synthetically by cloning un-rearranged V-gene segments from stem cells, and using PCR primers containing random sequences to amplify the highly variable CDR3 regions and to accomplish rearrangement in vitro. Antibodies or antibody fragments isolated from human antibody libraries are considered human antibodies or human antibody fragments herein.

In certain embodiments, an antibody provided herein is a multispecific antibody, e.g., a bispecific antibody. Multispecific antibodies are monoclonal antibodies that have binding specificities for at least two different sites or at least two different antigens. For example, one of the binding specificities can be to a fusion polypeptide of the disclosure, and the other can be to any other antigen. Multispecific antibodies can be prepared as full length antibodies or as antibody fragments. Techniques for making multispecific antibodies are known in the art and include, but are not limited to, recombinant co-expression of two immunoglobulin heavy chain-light chain pairs having different specificities, and “knob-in-hole” engineering. Multispecific antibodies may also be made by engineering electrostatic steering effects (e.g., by introducing mutations in the constant region) for making heterodimeric Fes; cross-linking two or more antibodies or fragments; using leucine zippers to produce bispecific antibodies; using “diabody” technology for making bispecific antibody fragments; using single-chain Fv (scFv) dimers; and preparing trispecific antibodies. Engineered antibodies with three or more functional antigen binding sites, including “Octopus antibodies,” are also included in the disclosure. Antibodies or antibody fragments of the disclosure also include “Dual Acting FAbs” or “DAF,” e.g., comprising an antigen binding site that binds to a fusion polypeptide of the disclosure as well as another, different antigen.

In certain embodiments, amino acid sequence variants of the antibodies provided herein are contemplated. For example, it may be desirable to improve the binding affinity and/or other biological properties of the antibody. Amino acid sequence variants of an antibody of the disclosure may be prepared by introducing appropriate modifications into the nucleotide sequence encoding the antibody, or by peptide synthesis. Such modifications include, for example, deletions, and/or insertions, and/or substitutions of residues within the amino acid sequences of the antibody. Any combination of deletions, insertions, and substitutions can be made to arrive at the final antibody, provided that the final antibody possesses the desired characteristics, e.g., antigen-binding.

In certain embodiments, antibody variants having one or more amino acid substitutions are provided. Sites of interest for substitutional mutagenesis include the HVRs and FRs. Amino acid substitutions may be introduced into an antibody of interest, and the products may be screened for a desired activity, e.g., retained/improved antigen binding, decreased immunogenicity, or improved or reduced antibody-dependent cell-mediated cytotoxicity (ADCC) and/or complement-dependent cytotoxicity (CDC).

In certain embodiments, an antibody of the present disclosure is altered to increase or to decrease the extent to which the antibody is glycosylated. Addition or deletion of glycosylation sites to an antibody may be conveniently accomplished by altering the amino acid sequence of the antibody, such that one or more glycosylation sites is created or removed. Antibody variants having bisected oligosaccharides are further provided, e.g., in which a biantennary oligosaccharide attached to the Fc region of the antibody is bisected by GlcNAc. In some embodiments, antibody variants of the disclosure may have increased fucosylation. In some embodiments, antibody variants of the disclosure may have reduced fucosylation. In some embodiments, antibody variants of the disclosure may have improved ADCC function. In some embodiments, antibody variants of the disclosure may have decreased ADCC function. Antibody variants with at least one galactose residue in the oligosaccharide attached to the Fc region are also provided. Such antibody variants may have improved CDC function. In some embodiments, antibody variants of the disclosure may have increased CDC function. In some embodiments, antibody variants of the disclosure may have decreased CDC function.

In certain embodiments, one or more amino acid modifications may be introduced into the Fc region of an antibody of the present disclosure, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the present disclosure contemplates an antibody variant that possesses some but not all effector functions, which make it a desirable candidate for applications in which the half-life of the antibody in vivo is important, yet certain effector functions (such as CDC and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks Fc-gamma-R binding (hence likely lacking ADCC activity), but retains FcRn binding ability. The primary cells that mediate ADCC, e.g., NK cells, express Fc-gamma-RIII only, whereas monocytes express Fc-gamma-RI, Fc-gamma-RII and Fc-gamma-RIII. Antibodies with reduced effector function include those with substitution of one or more of Fc region residues 238, 265, 269, 270, 297, 327 and 329. Such Fc mutants include Fc mutants with substitutions at two or more of amino acid positions 265, 269, 270, 297 and 327, including the so-called “DANA” Fc mutant with substitutions of residues 265 and 297 to alanine. Antibody variants with improved or diminished binding to FcRs are also included in the disclosure. In certain embodiments, an antibody variant comprises an Fc region with one or more amino acid substitutions that improve ADCC, e.g., substitutions at positions 298, 333, and/or 334 of the Fc region. In some embodiments, numbering of Fc region residues is according to EU numbering of residues. In some embodiments, alterations are made in the Fc region that result in altered (i.e., either improved or diminished) Clq binding and/or CDC. In some embodiments, antibodies of the disclosure include antibodies with increased half-lives and improved binding to the neonatal Fc receptor (FcRn), e.g., comprising one or more substitutions that improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434. See, also, Duncan & Winter, Nature 322:738-40 (1988); U.S. Pat. Nos. 5,648,260; 5,624,821; and WO 94/29351 for other examples of Fc region variants.

In certain embodiments, an antibody provided herein is a cysteine-engineered antibody, e.g., “thioMAb,” in which one or more residues of the antibody are substituted with cysteine residues. In some embodiments, the substituted residues occur at accessible sites of the antibody. By substituting those residues with cysteine, reactive thiol groups are thereby positioned at accessible sites of the antibody, and may be used to conjugate the antibody to other moieties, such as drug moieties or linker-drug moieties, e.g., to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine-engineered antibodies may be generated using any suitable method known in the art.

In some embodiments, an antibody or antibody fragment provided herein comprises a label or a tag. In some embodiments, the label or tag is a radiolabel, a fluorescent label, an enzymatic label, a sequence tag, biotin, or other ligands. Examples of labels or tags include, but are not limited to, 6xHis-tag, biotin-tag, Glutathione-S-transferase (GST)-tag, green fluorescent protein (GFP)-tag, c-myc-tag, FLAG-tag, Thioredoxin-tag, Glu-tag, Nus-tag, V5-tag, calmodulin-binding protein (CBP)-tag, Maltose binding protein (MBP)-tag, Chitin-tag, alkaline phosphatase (AP)-tag, HRP-tag, Biotin Caboxyl Carrier Protein (BCCP)-tag, Calmodulin-tag, S-tag, Strep-tag, haemoglutinin (HA)-tag, digoxigenin (DIG)-tag, DsRed, RFP, Luciferase, Short Tetracysteine Tags, Halo-tag, and Nus-tag. In some embodiments, the label or tag comprises a detection agent, such as a fluorescent molecule or an affinity reagent or tag.

In some embodiments, an antibody or antibody fragment provided herein is conjugated to a drug molecule, e.g., an anti-cancer agent described herein, or a cytotoxic agent such as mertansine or monomethyl auristatin E (MMAE).

In certain embodiments, an antibody or antibody fragment provided herein may be further modified to contain additional nonproteinaceous moieties. Such moieties may be suitable for derivatization of the antibody, e.g., including but not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyamino acids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, polyethylene glycol propionaldehyde, and mixtures thereof. The polymers may be of any molecular weight, and may be branched or unbranched. The number of polymers attached to the antibody may vary, and if more than one polymer is attached, the polymers can be the same or different molecules. In general, the number and/or type of polymers used for derivatization can be determined based on considerations including, but not limited to, the particular properties or functions of the antibody to be improved, or whether the antibody derivative will be used in a therapy under defined conditions. In some embodiments, provided herein are antibodies conjugated to carbon nanotubes, e.g., for use in methods to selectively heat the antibody using radiation to a temperature at which cells proximal to the antibody are killed.

### (iv) Samples

A variety of materials can be the source of, or serve as, samples for use in any of the methods of the disclosure, such as the methods for detection of a fusion nucleic acid molecule or a fusion polypeptide of the disclosure, or fragments thereof.

For example, the sample can be, or be derived from: solid tissue such as from a fresh, frozen and/or preserved organ, tissue sample, biopsy (e.g., tumor, tissue or liquid biopsy), resection, smear, or aspirate; scrapings; bone marrow or bone marrow specimens; a bone marrow aspirate; blood or any blood constituents; blood cells; bodily fluids such as cerebrospinal fluid, amniotic fluid, urine, saliva, sputum, peritoneal fluid or interstitial fluid; pleural fluid; ascites; tissue or fine needle biopsy samples; surgical specimens; cell-containing body fluids; free-floating nucleic acids; feces; lymph; gynecological fluids; skin swabs; vaginal swabs; oral swabs; nasal swabs; washings or lavages such as ductal lavages or bronchoalveolar lavages; cells from any time in gestation or development of an individual; cells from a cancer or tumor; other body fluids, secretions, and/or excretions, and/or cells therefrom. In some embodiments, a sample is or comprises cells obtained from an individual. In some embodiments, the sample is or is derived from blood or blood constituents, e.g., obtained from a liquid biopsy. In some embodiments, the sample is or is derived from a tumor sample. In some embodiments, the sample is or comprises biological tissue or fluid. In some embodiments, the sample can contain compounds that are not naturally intermixed with the source of the sample in nature, such as preservatives, anticoagulants, buffers, fixatives, nutrients, antibiotics or the like. In some embodiments, the sample is preserved as a frozen sample or as a formaldehyde- or paraformaldehyde-fixed paraffin-embedded (FFPE) tissue preparation. In some embodiments, the sample comprises circulating tumor cells (CTCs).

In one embodiment, the sample comprises one or more cells associated with a tumor, e.g., tumor cells or tumor-infiltrating lymphocytes (TIL). In one embodiment, the sample includes one or more premalignant or malignant cells. In one embodiment, the sample is acquired from a hematologic malignancy (or pre-malignancy), e.g., a hematologic malignancy (or pre-malignancy) described herein. In one embodiment, the sample is acquired from a cancer, such as a cancer described herein. In some embodiments, the sample is acquired from a solid tumor, a soft tissue tumor or a metastatic lesion. In other embodiments, the sample includes tissue or cells from a surgical margin. In one embodiment, the sample is or is acquired from a liquid biopsy of blood, plasma, cerebrospinal fluid, sputum, stool, urine, or saliva. In some embodiments, the sample includes cell-free DNA (cfDNA) and/or circulating tumor DNA (ctDNA), e.g., from a biopsy of blood, plasma, cerebrospinal fluid, sputum, stool, urine, or saliva. In another embodiment, the sample includes one or more circulating tumor cells (CTCs) (e.g., a CTC acquired from a blood sample). In one embodiment, the sample is a cell not associated with a tumor or cancer, e.g., a non-tumor or non-cancer cell or a peripheral blood lymphocyte.

In some embodiments, a sample is a primary sample obtained directly from a source of interest by any appropriate means. For example, in some embodiments, a primary biological sample is obtained by a method chosen from biopsy (e.g., fine needle aspiration or tissue biopsy), surgery, or collection of body fluid (e.g., blood, lymph, or feces). In some embodiments, as will be clear from context, the term “sample” refers to a preparation that is obtained by processing (e.g., by removing one or more components of and/or by adding one or more agents to) a primary sample. Such a processed sample may comprise, for example, nucleic acids (e.g., for use in any of the methods for detection of fusion nucleic acid molecules provided herein) or proteins (e.g., for use in any of the methods for detection of fusion polypeptides provided herein) extracted from a sample or obtained by subjecting a primary sample to techniques such as amplification methods, reverse transcription of mRNA, or isolation and/or purification of certain components such as nucleic acids and/or proteins.

In some embodiments, the sample comprises nucleic acids, e.g., genomic DNA, cDNA, or mRNA. In some embodiments, the sample comprises cell-free DNA (cfDNA). In some embodiments, the sample comprises cell-free RNA (cfRNA). In some embodiments, the sample comprises circulating tumor DNA (ctDNA). In certain embodiments, the nucleic acids are purified or isolated (e.g., removed from their natural state). In some embodiments, the sample comprises tumor or cancer nucleic acids, such as nucleic acids from a tumor or cancer sample, e.g., genomic DNA, RNA, or cDNA derived from RNA, or from a liquid biopsy, e.g., ctDNA from blood, plasma, cerebrospinal fluid, sputum, stool, urine, or saliva. In certain embodiments, a tumor or cancer nucleic acid sample, or a ctDNA sample, is purified or isolated (e.g., it is removed from its natural state).

In some embodiments, the sample comprises tumor or cancer proteins or polypeptides, such as proteins or polypeptides from a tumor or a cancer sample, or from a liquid biopsy, e.g., from blood, plasma, cerebrospinal fluid, sputum, stool, urine, or saliva. In certain embodiments, the proteins or polypeptides are purified or isolated (e.g., removed from their natural state).

In some embodiments, the sample is obtained from an individual having a cancer, such as a cancer described herein. In some embodiments, the sample comprises a fusion nucleic acid molecule or a fusion polypeptide of the disclosure.

In some embodiments, the sample is a control sample or a reference sample, e.g., not containing a fusion nucleic acid molecule or a fusion polypeptide described herein. In certain embodiments, the reference sample is purified or isolated (e.g., it is removed from its natural state). In certain embodiments, the reference or control sample comprises a wild type or a non-mutated nucleic acid molecule or polypeptide counterpart to any of the fusion nucleic acid molecules or fusion polypeptides described herein. In other embodiments, the reference sample is from a non-tumor or cancer sample, e.g., a blood control, a normal adjacent tumor (NAT), or any other non-cancerous sample from the same or a different individual.

In some embodiments, a fusion nucleic acid molecule of the disclosure is detected in a sample comprising genomic or subgenomic DNA fragments, or RNA (e.g., mRNA), isolated from a sample, e.g., a tumor or cancer sample, a normal adjacent tissue (NAT) sample, a tissue sample, or a blood, plasma, cerebrospinal fluid, sputum, stool, urine, or saliva sample obtained from an individual. In some embodiments, the sample comprises cDNA derived from an mRNA sample or from a sample comprising mRNA. In some embodiments, a fusion nucleic acid molecule of the disclosure is detected in a sample comprising cell-free DNA (cfDNA), cell-free RNA, and/or circulating tumor DNA (ctDNA). In some embodiments, a fusion nucleic acid molecule of the disclosure is detected in a sample comprising cell-free DNA (cfDNA) and/or circulating tumor DNA (ctDNA). In some embodiments, a fusion nucleic acid molecule of the disclosure is detected in a sample comprising circulating tumor DNA (ctDNA).

### C. Anti-Cancer Therapies

Certain aspects of the present disclosure relate to anti-cancer therapies, as well as methods for identifying an individual having a cancer who may benefit from a treatment comprising an anti-cancer therapy; selecting a treatment for an individual having a cancer; identifying one or more treatment options for an individual having a cancer; predicting survival of an individual having a cancer; treating or delaying progression of cancer; monitoring, evaluating or screening an individual having a cancer; detecting the presence or absence of a cancer in an individual; monitoring progression or recurrence of a cancer in an individual; or identifying a candidate treatment for a cancer in an individual in need thereof. The present disclosure also provides uses for anti-cancer therapies (e.g., in methods of treating or delaying progression of cancer in an individual, or in methods for manufacturing a medicament for treating or delaying progression of cancer). In some instances, the methods of the disclosure can include administering an anti-cancer therapy or applying an anti-cancer therapy to an individual based on a generated genomic and/or sequencing mutation profile. An anti-cancer therapy can refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

In some embodiments, an anti-cancer therapy of the disclosure is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for cancer comprising a fusion nucleic acid molecule or polypeptide of the disclosure, a treatment for cancer being tested in a clinical trial, a targeted therapy, a treatment being tested in a clinical trial for cancer comprising a fusion nucleic acid molecule or polypeptide of the disclosure, or any combination thereof, e.g., a described in further detail below. In some embodiments, the anti-cancer therapy is a kinase inhibitor, such as a kinase inhibitor described herein or known in the art. In some embodiments, the kinase inhibitor is a multi-kinase inhibitor or an ALK-, BRAF-, EGFR-, ERBB2-, FGFR1-, FGFR2-, FGFR3-, MET-, NTRK1-, RAF1-, RET-, or ROS1-specific inhibitor known in the art or described herein. In some embodiments, the nucleic acid inhibits the expression of a fusion nucleic acid molecule or polypeptide of the disclosure (e.g., an ALK, BRAF, EGFR, ERBB2, FGFR1, FGFR2, FGFR3, MET, NTRK1, RAF1, RET, or ROS1 fusion nucleic acid molecule described herein and/or in Tables 1-6, and/or in the Examples herein; or a fusion polypeptide encoded by such a fusion nucleic acid molecule).

In some embodiments, an anti-cancer therapy of the disclosure is an ALK-targeted therapy, e.g., as described herein or known in the art. In some embodiments, the ALK-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for ALK-positive or ALK-rearranged cancer, an ALK-targeted therapy being tested in a clinical trial, a treatment for ALK-positive or ALK-rearranged cancer being tested in a clinical trial, or any combination thereof. In some embodiments, the ALK-targeted therapy is a kinase inhibitor known in the art or described herein. In some embodiments, the ALK-targeted therapy is a tyrosine kinase inhibitor known in the art or described herein. In some embodiments, the ALK-targeted therapy is a multi-kinase inhibitor or an ALK-specific inhibitor known in the art or described herein. In some embodiments, the kinase inhibitor inhibits the kinase activity of an ALK polypeptide. In some embodiments, the ALK-targeted therapy comprises one or more of crizotinib, alectinib (AF802, CH5424802), ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, TAE684 (NVP-TAE684), CT-707, WX-0593, alkotinib, SIM1803-1A, PLB1003, SAF-189s, PF03446962, TQ-B3101, APG-2449, X-376, CEP-28122, and GSK1838705A. In some embodiments, the nucleic acid inhibits the expression of an ALK fusion nucleic acid molecule or polypeptide of the disclosure. In some embodiments, the ALK-targeted therapy is an ALK kinase inhibitor, e.g., as described in examples 3-39 of WO2005016894, which is incorporated herein by reference.

In some embodiments, an anti-cancer therapy of the disclosure is a BRAF-targeted therapy known in the art or described herein. In some embodiments, the BRAF-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for BRAF-rearranged cancer, a BRAF-targeted therapy being tested in a clinical trial, a treatment for BRAF-rearranged cancer being tested in a clinical trial, or any combination thereof. In some embodiments, the BRAF-targeted therapy is a kinase inhibitor known in the art or described herein. In some embodiments, the BRAF-targeted therapy is a serine/threonine kinase inhibitor known in the art or described herein. In some embodiments, the BRAF-targeted therapy is a multi-kinase inhibitor or a BRAF-specific inhibitor known in the art or described herein. In some embodiments, the kinase inhibitor inhibits the kinase activity of a BRAF polypeptide. In some embodiments, the BRAF-targeted therapy comprises one or more of sorafenib, PLX4720, PLX-3603, dabrafenib (GSK2118436), encorafenib (LGX818), GDC-0879, RAF265, XL281, ARQ736, BAY73-4506, vemurafenib (e.g., Zelboraf®), cobimetinib (e.g., Cotellic®), binimetinib (e.g., Mektovi®), regorafenib (e.g., Stivarga®), selumetinib (e.g., Koselugo®), trametinib (e.g., Mekinist®), or BAY 43-9006. In some embodiments, the nucleic acid inhibits the expression of a BRAF fusion nucleic acid molecule or polypeptide of the disclosure.

In some embodiments, an anti-cancer therapy of the disclosure is an EGFR-targeted therapy known in the art or described herein. In some embodiments, the EGFR-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for an EGFR-rearranged cancer, an EGFR-targeted therapy being tested in a clinical trial, a treatment for EGFR-rearranged cancer being tested in a clinical trial, or any combination thereof. In some embodiments, the EGFR-targeted therapy is a kinase inhibitor known in the art or described herein. In some embodiments, the EGFR-targeted therapy is a tyrosine kinase inhibitor known in the art or described herein. In some embodiments, the EGFR-targeted therapy is a multi-kinase inhibitor or an EGFR-specific inhibitor known in the art or described herein. In some embodiments, the kinase inhibitor inhibits the kinase activity of an EGFR polypeptide. In some embodiments, the EGFR-targeted therapy comprises one or more of cetuximab, panitumumab, lapatinib, gefitinib, vandetanib, dacomitinib, icotinib, osimertinib (AZD9291), afatanib, olmutinib, EGF816 (nazartinib), avitinib (AC00010), rociletinib (CO-1686), BMS-690514, YH5448, PF-06747775, ASP8273, PF299804, AP26113, necitumumab (e.g., Portrazza®), or erlotinib. In some embodiments, the nucleic acid inhibits the expression of an EGFR fusion nucleic acid molecule or polypeptide of the disclosure.

In some embodiments, an anti-cancer therapy of the disclosure is an ERBB2-targeted therapy known in the art or described herein. In some embodiments, the ERBB2-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for an ERBB2-rearranged cancer, an ERBB2-targeted therapy being tested in a clinical trial, a treatment for ERBB2-rearranged cancer being tested in a clinical trial, or any combination thereof. In some embodiments, the ERBB2-targeted therapy is a kinase inhibitor known in the art or described herein. In some embodiments, the ERBB2-targeted therapy is a tyrosine kinase inhibitor known in the art or described herein. In some embodiments, the ERBB2-targeted therapy is a multi-kinase inhibitor or an ERBB2-specific inhibitor known in the art or described herein. In some embodiments, the kinase inhibitor inhibits the kinase activity of an ERBB2 polypeptide. In some embodiments, the ERBB2-targeted therapy comprises one or more of afatinib, TAK-285, neratinib, dacomitinib, BMS-690514, BMS-599626, pelitinib, CP-724714, lapatinib, TAK-165, ARRY-380, AZD8931, AV-203, AMG-888, MM-111, MM-121, MM-141, LJM716, REGN1400, MEHD7945A, RG7116, trastuzumab, trastuzumab emtansine (T-DM1), pertuzumab, canertinib (CI-1033), or APC 8024. In some embodiments, the nucleic acid inhibits the expression of an ERBB2 fusion nucleic acid molecule or polypeptide of the disclosure.

In some embodiments, an anti-cancer therapy of the disclosure is an FGFR1-targeted therapy known in the art or described herein. In some embodiments, the FGFR1-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for an FGFR1-rearranged cancer, an FGFR1-targeted therapy being tested in a clinical trial, a treatment for FGFR1-rearranged cancer being tested in a clinical trial, or any combination thereof. In some embodiments, the FGFR1-targeted therapy is a kinase inhibitor known in the art or described herein. In some embodiments, the FGFR1-targeted therapy is a tyrosine kinase inhibitor known in the art or described herein. In some embodiments, the FGFR1-targeted therapy is a multi-kinase inhibitor or an FGFR1-specific inhibitor known in the art or described herein. In some embodiments, the kinase inhibitor inhibits the kinase activity of an FGFR1 polypeptide. In some embodiments, the FGFR1-targeted therapy comprises one or more of E3810 (lucitanib), AZD4547, Dovitinib (TKI258), Ponatinib, Derazantinib (ARQ 087), Nintendanib (BIBF1120), Rogaratinib (BAY 1163877), 3D185, SOMCL-085, brivanib (BMS582664), lenvatinib (E7080), orantinib (TSU-68), PRN1371, XL-228, AZ12908010 (AZ8010), Debio-1347 (CH5183284), FIIN-2, LY2874455, Infigratinib (BGJ398, NVP-BGJ398), Pemigatinib (e.g., Pemazyre®, INCB054828), Erdafitinib (JNJ-42756493, e.g., Balversa®), ASP5878, TAS-120, PRN1371, pazopanib (e.g., Votrient®), regorafenib (e.g., Stivarga®), or PKC412. In some embodiments, the nucleic acid inhibits the expression of an FGFR1 fusion nucleic acid molecule or polypeptide of the disclosure.

In some embodiments, an anti-cancer therapy of the disclosure is an FGFR2-targeted therapy known in the art or described herein. In some embodiments, the FGFR2-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for an FGFR2-rearranged cancer, an FGFR2-targeted therapy being tested in a clinical trial, a treatment for FGFR2-rearranged cancer being tested in a clinical trial, or any combination thereof. In some embodiments, the FGFR2-targeted therapy is a kinase inhibitor known in the art or described herein. In some embodiments, the FGFR2-targeted therapy is a tyrosine kinase inhibitor known in the art or described herein. In some embodiments, the FGFR2-targeted therapy is a multi-kinase inhibitor or an FGFR2-specific inhibitor known in the art or described herein. In some embodiments, the kinase inhibitor inhibits the kinase activity of an FGFR2 polypeptide. In some embodiments, the FGFR2-targeted therapy comprises one or more of E3810 (lucitanib), AZD4547, Dovitinib (TKI258), Ponatinib, Derazantinib (ARQ 087), Nintendanib (BIBF1120), Rogaratinib (BAY 1163877), 3D185, SOMCL-085, brivanib (BMS582664), lenvatinib (E7080), orantinib (TSU-68), PRN1371, XL-228, AZ12908010 (AZ8010), Debio-1347 (CH5183284), FIIN-2, LY2874455, Infigratinib (BGJ398, NVP-BGJ398), Pemigatinib (e.g., Pemazyre®, INCB054828), Erdafitinib (JNJ-42756493, e.g., Balversa®), ASP5878, TAS-120, PRN1371, formononetin, R04383596, Ki23057, SU5402, RLY-4008, pazopanib (e.g., Votrient®), regorafenib (e.g., Stivarga®), or PKC412. In some embodiments, the nucleic acid inhibits the expression of an FGFR2 fusion nucleic acid molecule or polypeptide of the disclosure.

In some embodiments, an anti-cancer therapy of the disclosure is an FGFR3-targeted therapy known in the art or described herein. In some embodiments, the FGFR3-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for an FGFR3-rearranged cancer, an FGFR3-targeted therapy being tested in a clinical trial, a treatment for FGFR3-rearranged cancer being tested in a clinical trial, or any combination thereof. In some embodiments, the FGFR3-targeted therapy is a kinase inhibitor known in the art or described herein. In some embodiments, the FGFR3-targeted therapy is a tyrosine kinase inhibitor known in the art or described herein. In some embodiments, the FGFR3-targeted therapy is a multi-kinase inhibitor or an FGFR3-specific inhibitor known in the art or described herein. In some embodiments, the kinase inhibitor inhibits the kinase activity of an FGFR3 polypeptide. In some embodiments, the FGFR3-targeted therapy comprises one or more of E3810 (lucitanib), AZD4547, Dovitinib (TKI258), Ponatinib, Derazantinib (ARQ 087), Nintendanib (BIBF1120), Rogaratinib (BAY 1163877), 3D185, SOMCL-085, brivanib (BMS582664), lenvatinib (E7080), orantinib (TSU-68), PRN1371, XL-228, AZ12908010 (AZ8010), Debio-1347 (CH5183284), FIIN-2, LY2874455, Infigratinib (BGJ398, NVP-BGJ398), Pemigatinib (e.g., Pemazyre®, INCB054828), Erdafitinib (JNJ-42756493, e.g., Balversa®), ASP5878, TAS-120, PRN1371, PKC412, Vofatamab (B-70), pazopanib (e.g., Votrient®), or MFGR1877S. In some embodiments, the nucleic acid inhibits the expression of an FGFR3 fusion nucleic acid molecule or polypeptide of the disclosure.

In some embodiments, an anti-cancer therapy of the disclosure is a MET-targeted therapy known in the art or described herein. In some embodiments, the MET-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for a MET-rearranged cancer, a MET-targeted therapy being tested in a clinical trial, a treatment for MET-rearranged cancer being tested in a clinical trial, or any combination thereof. In some embodiments, the MET-targeted therapy is a kinase inhibitor known in the art or described herein. In some embodiments, the MET-targeted therapy is a tyrosine kinase inhibitor known in the art or described herein. In some embodiments, the MET-targeted therapy is a multi-kinase inhibitor or a MET-specific inhibitor known in the art or described herein. In some embodiments, the kinase inhibitor inhibits the kinase activity of a MET polypeptide. In some embodiments, the MET-targeted therapy comprises tivantinib, cabozantinib, crizotinib, PHA-665752 and/or capmatinib (INC280). In some embodiments, the nucleic acid inhibits the expression of a MET fusion nucleic acid molecule or polypeptide of the disclosure.

In some embodiments, an anti-cancer therapy of the disclosure is an NTRK1-targeted therapy known in the art or described herein. In some embodiments, the NTRK1-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for an NTRK1-rearranged cancer, an NTRK1-targeted therapy being tested in a clinical trial, a treatment for NTRK1-rearranged cancer being tested in a clinical trial, or any combination thereof. In some embodiments, the NTRK1-targeted therapy is a kinase inhibitor known in the art or described herein. In some embodiments, the NTRK1-targeted therapy is a tyrosine kinase inhibitor known in the art or described herein. In some embodiments, the NTRK1-targeted therapy is a multi-kinase inhibitor or an NTRK1-specific inhibitor known in the art or described herein. In some embodiments, the kinase inhibitor inhibits the kinase activity of an NTRK1 polypeptide. In some embodiments, the NTRK1-targeted therapy comprises altiratinib (DCC-2701). In some embodiments, the NTRK1-targeted therapy comprises one or more of altiratinib (DCC-2701), AG 879 (Tyrphostin AG 879), an anti-TrK antibody, ARRY 954, AR523, AZ-23, AZ623, a benzotriazole, CEP-2563, danusertib (PHA-739358), entrectinib (also known as RXDX-101 or NMS-E628), DS-6051, GNF 5837, GW 441756, indenopyrrolocarboazole 12a, isothiazole 5n, larotrectinib (previously known as LOXO-101 or ARRY-470), lestaurtinib (CEP-701), selitrectinib (LOXO-195), a macrocyclic compound, ONO-5390556, oxindole 3, pegcantratinib (SNA-120), PHA-848125, PLX7486, a pyrazole derivative, a pyrazolo[1; 5a]pyrimidine, a pyridocarbazole, a pyridoquinazolinyl, a pyridotriazole, a pyrrolidinyl thiourea, a pyrrolidinyl urea, a pyrrolo[2; 3-d]pyrimidine, a quinazolinyl, repotrectinib (TPX-0005), Ro 08-2750, a substituted pyrazolo[1; 5a]pyrimidine, sitravatinib (MGCD516), SNA-125, tavilermide, thiazole 20h, F17752, cabozantinib (XL184), merestinib (LY2801653), belizatinib (TSR-011), dovitinib, ONO-7579, and/or VMD-928.

In some embodiments, an anti-cancer therapy of the disclosure is a RAF1-targeted therapy known in the art or described herein. In some embodiments, the RAF1-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for a RAF1-rearranged cancer, a RAF1-targeted therapy being tested in a clinical trial, a treatment for RAF1-rearranged cancer being tested in a clinical trial, or any combination thereof. In some embodiments, the RAF1-targeted therapy is a kinase inhibitor known in the art or described herein. In some embodiments, the RAF1-targeted therapy is a serine/threonine kinase inhibitor known in the art or described herein. In some embodiments, the RAF1-targeted therapy is a multi-kinase inhibitor or a RAF1-specific inhibitor known in the art or described herein. In some embodiments, the kinase inhibitor inhibits the kinase activity of a RAF1 polypeptide. In some embodiments, the RAF1-targeted therapy comprises one or more of Sorafenib (BAY49-9006), Binimetinib (e.g., Mektovi®), Cobimetinib (e.g., Cotellic®), Regorafenib (e.g., Stivarga®), Trametinib (e.g., Mekinit®), or RAF265. In some embodiments, the nucleic acid inhibits the expression of a RAF1 fusion nucleic acid molecule or polypeptide of the disclosure.

In some embodiments, an anti-cancer therapy of the disclosure is a RET-targeted therapy known in the art or described herein. In some embodiments, the RET-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for a RET-rearranged cancer, a RET-targeted therapy being tested in a clinical trial, a treatment for RET-rearranged cancer being tested in a clinical trial, or any combination thereof. In some embodiments, the RET-targeted therapy is a kinase inhibitor known in the art or described herein. In some embodiments, the RET-targeted therapy is a tyrosine kinase inhibitor known in the art or described herein. In some embodiments, the RET-targeted therapy is a multi-kinase inhibitor or a RET-specific inhibitor known in the art or described herein. In some embodiments, the kinase inhibitor inhibits the kinase activity of a RET polypeptide. In some embodiments, the RET-targeted therapy comprises one or more of Selpercatinib (e.g., Retevmo®), Pralsetinib (e.g., Gavreto®), Alectinib (e.g., Alecensa®), Cabozantinib (e.g., Cabometyx®), Lenvatinib (e.g., Lenvima®), Ponatinib (e.g., Iclusig®), Regorafenib (e.g., Stivarga®), Sorafenib (e.g., Nexavar®), Sunitinib (e.g., Sutent®), or Vandetanib (e.g., Caprelsa®).

In some embodiments, the nucleic acid inhibits the expression of a RET fusion nucleic acid molecule or polypeptide of the disclosure.

In some embodiments, an anti-cancer therapy of the disclosure is a ROS1-targeted therapy known in the art or described herein. In some embodiments, the ROS1-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for a ROS1-rearranged cancer, a ROS1-targeted therapy being tested in a clinical trial, a treatment for ROS1-rearranged cancer being tested in a clinical trial, or any combination thereof. In some embodiments, the ROS1-targeted therapy is a kinase inhibitor known in the art or described herein. In some embodiments, the ROS1-targeted therapy is a tyrosine kinase inhibitor known in the art or described herein. In some embodiments, the ROS1-targeted therapy is a multi-kinase inhibitor or a ROS1-specific inhibitor known in the art or described herein. In some embodiments, the kinase inhibitor inhibits the kinase activity of a ROS1 polypeptide. In some embodiments, the ROS1-targeted therapy comprises one or more of crizotinib (e.g., Xalkori®), lorlatinib (e.g., Lorbrena®), TQ-B3139, repotrectinib (TPX-0005), brigatinib (e.g., Alunbrig®), cabozantinib (e.g., Cabometyx®), ceritinib (e.g., Zykadia®), or entrectinib. In some embodiments, the nucleic acid inhibits the expression of a ROS1 fusion nucleic acid molecule or polypeptide of the disclosure.

In some embodiments, an anti-cancer therapy of the disclosure is administered in combination with an additional anti-cancer therapy. In some embodiments, the additional anti-cancer therapy is any anti-cancer therapy known in the art or described herein. In some embodiments, the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof.

In some embodiments, an anti-cancer therapy of the disclosure comprises a cyclin-dependent kinase (CDK) inhibitor, e.g., alone or in combination with an ALK-, BRAF-, EGFR-, ERBB2-, FGFR1-, FGFR2-, FGFR3-, MET-, NTRK1-, RAF1-, RET-, or ROS1-targeted therapy. In some embodiments, the CDK inhibitor inhibits CDK4. In some embodiments, the CDK inhibitor inhibits Cyclin D/CDK4. In some embodiments, the CDK inhibitor is (a) a small molecule that inhibits one or more enzymatic activities of CDK4, (b) an antibody that inhibits one or more activities of CDK4 (e.g., by binding to and inhibiting one or more activities of CDK4, binding to and inhibiting expression of CDK4, and/or binding to and inhibiting one or more activities of a cell expressing CDK4, such as by inducing antibody-dependent cellular cytotoxicity, ADCC, or phagocytosis, ADCP), or (c) a nucleic acid that inhibits expression of CDK4 (e.g., an antisense oligonucleotide, miRNA, siRNA, morpholino, CRISPR-based therapeutic, and the like). In some embodiments, the CDK inhibitor inhibits CDK4 and CDK6. In some embodiments, the CDK inhibitor is a small molecule inhibitor of CDK4 (e.g., a competitive or non-competitive inhibitor). Non-limiting examples of CDK inhibitors include palbociclib, ribociclib, and abemaciclib, as well as pharmaceutically acceptable salts thereof.

In some embodiments, an anti-cancer therapy of the disclosure comprises a murine double minute 2 homolog (MDM2) inhibitor, e.g., alone or in combination with an ALK-, BRAF-, EGFR-, ERBB2-, FGFR1-, FGFR2-, FGFR3-, MET-, NTRK1-, RAF1-, RET-, or ROS1-targeted therapy. In some embodiments, the MDM2 inhibitor is (a) a small molecule that inhibits one or more activities of MDM2 (e.g., binding to p53), (b) an antibody that inhibits one or more activities of MDM2 (e.g., by binding to and inhibiting one or more activities of MDM2, binding to and inhibiting expression of MDM2, and/or binding to and inhibiting one or more activities of a cell expressing MDM2, such as by inducing antibody-dependent cellular cytotoxicity, ADCC, or phagocytosis, ADCP), or (c) a nucleic acid that inhibits expression of MDM2 (e.g., an antisense oligonucleotide, miRNA, siRNA, morpholino, CRISPR-based therapeutic, and the like). In some embodiments, the MDM2 inhibitor is a small molecule inhibitor of MDM2 (e.g., a competitive or non-competitive inhibitor). Non-limiting examples of MDM2 inhibitors include nutlin-3a, RG7112, idasanutlin (RG7388), AMG-232, MI-63, MI-291, MI-391, MI-77301 (SAR405838), APG-115, DS-3032b, NVP-CGMO97, and HDM-201 (siremadlin), as well as pharmaceutically acceptable salts thereof. In some embodiments, the MDM2 inhibitor inhibits or disrupts interaction between MDM2 and p53.

In some embodiments, an anti-cancer therapy of the disclosure comprises (alone or in combination with an ALK-, BRAF-, EGFR-, ERBB2-, FGFR1-, FGFR2-, FGFR3-, MET-, NTRK1-, RAF1-, RET-, or ROS1-targeted therapy) one or more of an antimetabolite, DNA-damaging agent, or platinum-containing therapeutic (e.g., 5-azacitadine, 5-fluorouracil, acadesine, busulfan, carboplatin, cisplatin, chlorambucil, CPT-11, cytarabine, daunorubicin, decitabine, doxorubicin, etoposide, fludarabine, gemcitabine, idarubicin, radiation, oxaliplatin, temozolomide, topotecan, trabectedin, GSK2830371, or rucaparib); a pro-apoptotic agent (e.g., a BCL2 inhibitor or downregulator, SMAC mimetic, or TRAIL agonist such as ABT-263, ABT-737, oridonin, venetoclax, combination of venetoclax and an anti-CD20 antibody such as obinutuzumab or rituximab, 1396-11, ABT-10, SM-164, D269H/E195R, or rhTRAIL); a tyrosine kinase inhibitor (e.g., as described herein); an inhibitor of RAS, RAF, MEK, or the MAPK pathway (e.g., AZD6244, dabrafenib, LGX818, PD0325901, pimasertib, trametinib, or vemurafenib); an inhibitor of PI3K, mTOR, or Akt (e.g., as described herein); a CDK inhibitor (e.g., as described herein); a PKC inhibitor (e.g., LXS196 or sotrastaurin); an antibody-based therapeutic (e.g., an anti-PD-1 or anti-PDL1 antibody such as atezolizumab, pembrolizumab, nivolumab, or spartalizumab; an anti-CD20 antibody such as obinutuzumab or rituximab; or an anti-DR5 antibody such as drozitumab); a proteasome inhibitor (e.g., bortezomib, carfilzomib, ixazomib, or MG-132); an HDAC inhibitor (e.g., SAHA or VPA); an antibiotic (e.g., actinomycin D); a zinc-containing therapeutic (e.g., zinc or ZMC1); an HSP inhibitor (e.g., geldanamycin); an ATPase inhibitor (e.g., archazolid); a mitotic inhibitor (e.g., paclitaxel or vincristine); metformin; methotrexate; tanshinone IIA; and/or P5091.

In some embodiments, an anti-cancer therapy of the disclosure comprises a tyrosine kinase inhibitor, e.g., alone or in combination with an ALK-, BRAF-, EGFR-, ERBB2-, FGFR1-, FGFR2-, FGFR3-, MET-, NTRK1-, RAF1-, RET-, or ROS1-targeted therapy. In some embodiments, the tyrosine kinase inhibitor is (a) a small molecule that inhibits one or more enzymatic activities of a tyrosine kinase, (b) an antibody that inhibits one or more activities of a tyrosine kinase (e.g., by binding to and inhibiting one or more activities of the tyrosine kinase, binding to and inhibiting expression, such as cell surface expression, of the tyrosine kinase, and/or binding to and inhibiting one or more activities of a cell expressing the tyrosine kinase, such as by inducing antibody-dependent cellular cytotoxicity, ADCC, or phagocytosis, ADCP), or (c) a nucleic acid that inhibits expression of a tyrosine kinase (e.g., an antisense oligonucleotide, miRNA, siRNA, morpholino, CRISPR-based therapeutic, and the like). In some embodiments, the tyrosine kinase inhibitor is a small molecule inhibitor of a tyrosine kinase (e.g., a competitive or non-competitive inhibitor). Non-limiting examples of tyrosine kinase inhibitors include imatinib, crenolanib, linifanib, ninetedanib, axitinib, dasatinib, imetelstat, midostaurin, pazopanib, sorafenib, sunitinb, motesanib, masitinib, vatalanib, cabozanitinib, tivozanib, OSI-930, Ki8751, telatinib, dovitinib, tyrphostin AG 1296, and amuvatinib, as well as pharmaceutically acceptable salts thereof.

In some embodiments, an anti-cancer therapy of the disclosure comprises a mitogen-activated protein kinase (MEK) inhibitor, e.g., alone or in combination with an ALK-, BRAF-, EGFR-, ERBB2-, FGFR1-, FGFR2-, FGFR3-, MET-, NTRK1-, RAF1-, RET-, or ROS1-targeted therapy. In some embodiments, the MEK inhibitor inhibits one or more activities of MEK1 and/or MEK2. In some embodiments, the anti-cancer therapy/MEK inhibitor is (a) a small molecule that inhibits one or more enzymatic activities of MEK, (b) an antibody that inhibits one or more activities of MEK (e.g., by binding to and inhibiting one or more activities of MEK, binding to and inhibiting expression of MEK, and/or binding to and inhibiting one or more activities of a cell expressing MEK, such as by inducing antibody-dependent cellular cytotoxicity, ADCC, or phagocytosis, ADCP), or (c) a nucleic acid that inhibits expression of MEK (e.g., an antisense oligonucleotide, miRNA, siRNA, morpholino, CRISPR-based therapeutic, and the like). In some embodiments, the MEK inhibitor is a small molecule inhibitor of MEK (e.g., a competitive or non-competitive inhibitor). Non-limiting examples of MEK inhibitors include trametinib, cobimetinib, binimetinib, CI-1040, PD0325901, selumetinib, AZD8330, TAK-733, GDC-0623, refametinib, pimasertib, R04987655, R05126766, WX-544, and HL-085, as well as pharmaceutically acceptable salts thereof. In some embodiments, the anti-cancer therapy inhibits one or more activities of the Raf/MEK/ERK pathway, including inhibitors of Raf, MEK, and/or ERK.

In some embodiments, an anti-cancer therapy of the disclosure comprises a mammalian target of rapamycin (mTOR) inhibitor, e.g., alone or in combination with an ALK-, BRAF-, EGFR-, ERBB2-, FGFR1-, FGFR2-, FGFR3-, MET-, NTRK1-, RAF1-, RET-, or ROS1-targeted therapy. In some embodiments, the mTOR inhibitor is (a) a small molecule that inhibits one or more enzymatic activities of mTOR, (b) an antibody that inhibits one or more activities of mTOR (e.g., by binding to and inhibiting one or more activities of mTOR, binding to and inhibiting expression of mTOR, and/or binding to and inhibiting one or more activities of a cell expressing mTOR, such as by inducing antibody-dependent cellular cytotoxicity, ADCC, or phagocytosis, ADCP), or (c) a nucleic acid that inhibits expression of mTOR (e.g., an antisense oligonucleotide, miRNA, siRNA, morpholino, CRISPR-based therapeutic, and the like). In some embodiments, the mTOR inhibitor is a small molecule inhibitor of mTOR (e.g., a competitive inhibitor, such as an ATP-competitive inhibitor, or a non-competitive inhibitor, such as a rapamycin analog). Non-limiting examples of mTOR inhibitors include temsirolimus, everolimus, ridaforolimus, dactolisib, GSK2126458, XL765, AZD8055, AZD2014, MLN128, PP242, NVP-BEZ235, LY3023414, PQR309, PKI587, and OSI027, as well as pharmaceutically acceptable salts thereof. In some embodiments, the anti-cancer therapy inhibits one or more activities of the Akt/mTOR pathway, including inhibitors of Akt and/or mTOR.

In some embodiments, an anti-cancer therapy of the disclosure comprises a PI3K inhibitor or Akt inhibitor, e.g., alone or in combination with an ALK-, BRAF-, EGFR-, ERBB2-, FGFR1-, FGFR2-, FGFR3-, MET-, NTRK1-, RAF1-, RET-, or ROS1-targeted therapy. In some embodiments, the PI3K inhibitor inhibits one or more activities of PI3K. In some embodiments, the anti-cancer therapy/PI3K inhibitor is (a) a small molecule that inhibits one or more enzymatic activities of PI3K, (b) an antibody that inhibits one or more activities of PI3K (e.g., by binding to and inhibiting one or more activities of PI3K, binding to and inhibiting expression of PI3K, and/or binding to and inhibiting one or more activities of a cell expressing PI3K, such as by inducing antibody-dependent cellular cytotoxicity, ADCC, or phagocytosis, ADCP), or (c) a nucleic acid that inhibits expression of PI3K (e.g., an antisense oligonucleotide, miRNA, siRNA, morpholino, CRISPR-based therapeutic, and the like). In some embodiments, the PI3K inhibitor is a small molecule inhibitor of PI3K (e.g., a competitive or non-competitive inhibitor). Non-limiting examples of PI3K inhibitors include GSK2636771, buparlisib (BKM120), AZD8186, copanlisib (BAY80-6946), LY294002, PX-866, TGX115, TGX126, BEZ235, SF1126, idelalisib (GS-1101, CAL-101), pictilisib (GDC-094), GDCO0032, IPI145, INK1117 (MLN1117), SAR260301, KIN-193 (AZD6482), duvelisib, GS-9820, GSK2636771, GDC-0980, AMG319, pazobanib, and alpelisib (BYL719, Piqray), as well as pharmaceutically acceptable salts thereof. In some embodiments, the AKT inhibitor inhibits one or more activities of AKT (e.g., AKT1). In some embodiments, the AKT inhibitor is (a) a small molecule that inhibits one or more enzymatic activities of AKT1, (b) an antibody that inhibits one or more activities of AKT1 (e.g., by binding to and inhibiting one or more activities of AKT1, binding to and inhibiting expression of AKT1, and/or binding to and inhibiting one or more activities of a cell expressing AKT1, such as by inducing antibody-dependent cellular cytotoxicity, ADCC, or phagocytosis, ADCP), or (c) a nucleic acid that inhibits expression of AKT1 (e.g., an antisense oligonucleotide, miRNA, siRNA, morpholino, CRISPR-based therapeutic, and the like). In some embodiments, the AKT1 inhibitor is a small molecule inhibitor of AKT1 (e.g., a competitive or non-competitive inhibitor). Non-limiting examples of AKT1 inhibitors include GSK690693, GSK2141795 (uprosertib), GSK2110183 (afuresertib), AZD5363, GDC-0068 (ipatasertib), AT7867, CCT128930, MK-2206, BAY 1125976, AKT1 and AKT2-IN-1, perifosine, and VIII, as well as pharmaceutically acceptable salts thereof. In some embodiments, the AKT1 inhibitor is a pan-Akt inhibitor.

In some embodiments, an anti-cancer therapy of the disclosure comprises a hedgehog (Hh) inhibitor, e.g., alone or in combination with an ALK-, BRAF-, EGFR-, ERBB2-, FGFR1-, FGFR2-, FGFR3-, MET-, NTRK1-, RAF1-, RET-, or ROS1-targeted therapy. In some embodiments, the Hh inhibitor is (a) a small molecule that inhibits one or more enzymatic activities of Hh, (b) an antibody that inhibits one or more activities of Hh (e.g., by binding to and inhibiting one or more activities of Hh, binding to and inhibiting expression of Hh, and/or binding to and inhibiting one or more activities of a cell expressing Hh, such as by inducing antibody-dependent cellular cytotoxicity, ADCC, or phagocytosis, ADCP), or (c) a nucleic acid that inhibits expression of Hh (e.g., an antisense oligonucleotide, miRNA, siRNA, morpholino, CRISPR-based therapeutic, and the like). In some embodiments, the Hh inhibitor is a small molecule inhibitor of Hh (e.g., a competitive or non-competitive inhibitor). Non-limiting examples of Hh inhibitors include sonidegib, vismodegib, erismodegib, saridegib, BMS833923, PF-04449913, and LY2940680, as well as pharmaceutically acceptable salts thereof.

In some embodiments, an anti-cancer therapy of the disclosure comprises a heat shock protein (HSP) inhibitor, a MYC inhibitor, an HDAC inhibitor, an immunotherapy, a neoantigen, a vaccine, or a cellular therapy, e.g., alone or in combination with an ALK-, BRAF-, EGFR-, ERBB2-, FGFR1-, FGFR2-, FGFR3-, MET-, NTRK1-, RAF1-, RET-, or ROS1-targeted therapy.

In some embodiments, the anti-cancer therapy comprises one or more of an immune checkpoint inhibitor, a chemotherapy, a VEGF inhibitor, an Integrin β3 inhibitor, a statin, an EGFR inhibitor, an mTOR inhibitor, a PI3K inhibitor, a MAPK inhibitor, or a CDK4/6 inhibitor, e.g., alone or in combination with an ALK-, BRAF-, EGFR-, ERBB2-, FGFR1-, FGFR2-, FGFR3-, MET-, NTRK1-, RAF1-, RET-, or ROS1-targeted therapy.

In some embodiments, the anti-cancer therapy comprises a kinase inhibitor, e.g., alone or in combination with an ALK-, BRAF-, EGFR-, ERBB2-, FGFR1-, FGFR2-, FGFR3-, MET-, NTRK1-, RAF1-, RET-, or ROS1-targeted therapy. In some embodiments, the kinase inhibitor is crizotinib, alectinib, ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, or TAE684 (NVP-TAE684). In some embodiments, the kinase inhibitor is an ALK kinase inhibitor, e.g., as described in examples 3-39 of WO2005016894, which is incorporated herein by reference.

In some embodiments, the anti-cancer therapy comprises a heat shock protein (HSP) inhibitor, e.g., alone or in combination with an ALK-, BRAF-, EGFR-, ERBB2-, FGFR1-, FGFR2-, FGFR3-, MET-, NTRK1-, RAF1-, RET-, or ROS1-targeted therapy. In some embodiments, the HSP inhibitor is a Pan-HSP inhibitor, such as KNK423. In some embodiments, the HSP inhibitor is an HSP70 inhibitor, such as cmHsp70.1, quercetin, VER155008, or 17-AAD. In some embodiments, the HSP inhibitor is a HSP90 inhibitor. In some embodiments, the HSP90 inhibitor is 17-AAD, Debio0932, ganetespib (STA-9090), retaspimycin hydrochloride (retaspimycin, IPI-504), AUY922, alvespimycin (KOS-1022, 17-DMAG), tanespimycin (KOS-953, 17-AAG), DS 2248, or AT13387 (onalespib). In some embodiments, the HSP inhibitor is an HSP27 inhibitor, such as Apatorsen (OGX-427).

In some embodiments, the anti-cancer therapy comprises a MYC inhibitor, e.g., alone or in combination with an ALK-, BRAF-, EGFR-, ERBB2-, FGFR1-, FGFR2-, FGFR3-, MET-, NTRK1-, RAF1-, RET-, or ROS1-targeted therapy. In some embodiments, the MYC inhibitor is MYCi361 (NUCC-0196361), MYCi975 (NUCC-0200975), Omomyc (dominant negative peptide), ZINC16293153 (Min9), 10058-F4, JKY-2-169, 7594-0035, or inhibitors of MYC/MAX dimerization and/or MYC/MAX/DNA complex formation.

In some embodiments, the anti-cancer therapy comprises a histone deacetylase (HDAC) inhibitor, e.g., alone or in combination with an ALK-, BRAF-, EGFR-, ERBB2-, FGFR1-, FGFR2-, FGFR3-, MET-, NTRK1-, RAF1-, RET-, or ROS1-targeted therapy. In some embodiments, the HDAC inhibitor is belinostat (PXD101, e.g., Beleodaq®), SAHA (vorinostat, suberoylanilide hydroxamine, e.g., Zolinza®), panobinostat (LBH589, LAQ-824), ACY1215 (Rocilinostat), quisinostat (JNJ-26481585), abexinostat (PCI-24781), pracinostat (SB939), givinostat (ITF2357), resminostat (4SC-201), trichostatin A (TSA), MS-275 (etinostat), Romidepsin (depsipeptide, FK228), MGCD0103 (mocetinostat), BML-210, CAY10603, valproic acid, MC1568, CUDC-907, CI-994 (Tacedinaline), Pivanex (AN-9), AR-42, Chidamide (CS055, HBI-8000), CUDC-101, CHR-3996, MPT0E028, BRD8430, MRLB-223, apicidin, RGFP966, BG45, PCI-34051, C149 (NCC149), TMP269, Cpd2, T247, T326, LMK235, C1A, HPOB, Nexturastat A, Befexamac, CBHA, Phenylbutyrate, MC1568, SNDX275, Scriptaid, Merck60, PX089344, PX105684, PX117735, PX117792, PX117245, PX105844, compound 12 as described by Li et al., Cold Spring Harb Perspect Med (2016) 6(10):a026831, or PX117445.

In some embodiments, the anti-cancer therapy comprises a VEGF inhibitor, e.g., alone or in combination with an ALK-, BRAF-, EGFR-, ERBB2-, FGFR1-, FGFR2-, FGFR3-, MET-, NTRK1-, RAF1-, RET-, or ROS1-targeted therapy. In some embodiments, the VEGF inhibitor is Bevacizumab (e.g., Avastin®), BMS-690514, ramucirumab, pazopanib, sorafenib, sunitinib, golvatinib, vandetanib, cabozantinib, levantinib, axitinib, cediranib, tivozanib, lucitanib, semaxanib, nindentanib, regorafinib, or aflibercept.

In some embodiments, the anti-cancer therapy comprises an integrin β3 inhibitor, e.g., alone or in combination with an ALK-, BRAF-, EGFR-, ERBB2-, FGFR1-, FGFR2-, FGFR3-, MET-, NTRK1-, RAF1-, RET-, or ROS1-targeted therapy. In some embodiments, the integrin β3 inhibitor is anti-avb3 (clone LM609), cilengitide (EMD121974, NSC, 707544), an siRNA, GLPG0187, MK-0429, CNTO95, TN-161, etaracizumab (MEDI-522), intetumumab (CNTO95) (anti-alphaV subunit antibody), abituzumab (EMD 525797/DI17E6) (anti-alphaV subunit antibody), JSM6427, SJ749, BCH-15046, SCH221153, or SC56631. In some embodiments, the anti-cancer therapy comprises an αIIbβ3 integrin inhibitor, e.g., alone or in combination with an ALK-, BRAF-, EGFR-, ERBB2-, FGFR1-, FGFR2-, FGFR3-, MET-, NTRK1-, RAF1-, RET-, or ROS1-targeted therapy. In some embodiments, the αIIbβ3 integrin inhibitor is abciximab, eptifibatide (e.g., Integrilin®), or tirofiban (e.g., Aggrastat®).

In some embodiments, the anti-cancer therapy comprises an mTOR inhibitor, e.g., alone or in combination with an ALK-, BRAF-, EGFR-, ERBB2-, FGFR1-, FGFR2-, FGFR3-, MET-, NTRK1-, RAF1-, RET-, or ROS1-targeted therapy. In some embodiments, the mTOR inhibitor is temsirolimus (CCI-779), KU-006379, PP242, Torin1, Torin2, ICSN3250, Rapalink-1, CC-223, sirolimus (rapamycin), everolimus (RAD001), dactosilib (NVP-BEZ235), GSK2126458, WAY-001, WAY-600, WYE-687, WYE-354, SF1126, XL765, INK128 (MLN012), AZD8055, OSI027, AZD2014, or AP-23573.

In some embodiments, the anti-cancer therapy comprises a statin or a statin-based agent, e.g., alone or in combination with an ALK-, BRAF-, EGFR-, ERBB2-, FGFR1-, FGFR2-, FGFR3-, MET-, NTRK1-, RAF1-, RET-, or ROS1-targeted therapy. In some embodiments, the statin or statin-based agent is simvastatin, atorvastatin, fluvastatin, pitavastatin, pravastatin, rosuvastatin, or cerivastatin.

In some embodiments, the anti-cancer therapy comprises a MAPK inhibitor, e.g., alone or in combination with an ALK-, BRAF-, EGFR-, ERBB2-, FGFR1-, FGFR2-, FGFR3-, MET-, NTRK1-, RAF1-, RET-, or ROS1-targeted therapy. In some embodiments, the MAPK inhibitor is SB203580, SKF-86002, BIRB-796, SC-409, RJW-67657, BIRB-796, VX-745, R03201195, SB-242235, or MW181.

In some embodiments, the anti-cancer therapy comprises an EGFR inhibitor, e.g., alone or in combination with an ALK-, BRAF-, EGFR-, ERBB2-, FGFR1-, FGFR2-, FGFR3-, MET-, NTRK1-, RAF1-, RET-, or ROS1-targeted therapy. In some embodiments, the EGFR inhibitor is cetuximab, panitumumab, lapatinib, gefitinib, vandetanib, dacomitinib, icotinib, osimertinib (AZD9291), afatanib, olmutinib, EGF816 (nazartinib), avitinib (AC00010), rociletinib (CO-1686), BMS-690514, YH5448, PF-06747775, ASP8273, PF299804, AP26113, necitumumab (e.g., Portrazza®), or erlotinib. In some embodiments, the EGFR inhibitor is gefitinib or cetuximab.

In some embodiments, the anti-cancer therapy comprises a cancer immunotherapy, such as a checkpoint inhibitor, cancer vaccine, cell-based therapy, T cell receptor (TCR)-based therapy, adjuvant immunotherapy, cytokine immunotherapy, and oncolytic virus therapy, e.g., alone or in combination with an ALK-, BRAF-, EGFR-, ERBB2-, FGFR1-, FGFR2-, FGFR3-, MET-, NTRK1-, RAF1-, RET-, or ROS1-targeted therapy. In some embodiments, the cancer immunotherapy comprises a small molecule, nucleic acid, polypeptide, carbohydrate, toxin, cell-based agent, or cell-binding agent. Examples of cancer immunotherapies are described in greater detail herein but are not intended to be limiting. In some embodiments, the cancer immunotherapy activates one or more aspects of the immune system to attack a cell (e.g., a tumor cell) that expresses a neoantigen, e.g., a neoantigen corresponding to a fusion nucleic acid molecule or polypeptide of the disclosure. The cancer immunotherapies of the present disclosure are contemplated for use as monotherapies, or in combination approaches comprising two or more in any combination or number, subject to medical judgement. Any of the cancer immunotherapies (optionally as monotherapies or in combination with another cancer immunotherapy or other therapeutic agent described herein) may find use in any of the methods described herein.

In some embodiments, the cancer immunotherapy comprises a cancer vaccine, e.g., alone or in combination with an ALK-, BRAF-, EGFR-, ERBB2-, FGFR1-, FGFR2-, FGFR3-, MET-, NTRK1-, RAF1-, RET-, or ROS1-targeted therapy. A range of cancer vaccines have been tested that employ different approaches to promoting an immune response against a cancer (see, e.g., Emens L A, Expert Opin Emerg Drugs 13(2): 295-308 (2008) and US20190367613). Approaches have been designed to enhance the response of B cells, T cells, or professional antigen-presenting cells against tumors. Exemplary types of cancer vaccines include, but are not limited to, DNA-based vaccines, RNA-based vaccines, virus transduced vaccines, peptide-based vaccines, dendritic cell vaccines, oncolytic viruses, whole tumor cell vaccines, tumor antigen vaccines, etc. In some embodiments, the cancer vaccine can be prophylactic or therapeutic. In some embodiments, the cancer vaccine is formulated as a peptide-based vaccine, a nucleic acid-based vaccine, an antibody based vaccine, or a cell based vaccine. For example, a vaccine composition can include naked cDNA in cationic lipid formulations; lipopeptides (e.g., Vitiello, A. et al, J. Clin. Invest. 95:341, 1995), naked cDNA or peptides, encapsulated e.g., in poly(DL-lactide-co-glycolide) (“PLG”) microspheres (see, e.g., Eldridge, et ah, Molec. Immunol. 28:287-294, 1991: Alonso et al, Vaccine 12:299-306, 1994; Jones et al, Vaccine 13:675-681, 1995); peptide composition contained in immune stimulating complexes (ISCOMS) (e.g., Takahashi et al, Nature 344:873-875, 1990; Hu et al, Clin. Exp. Immunol. 113:235-243, 1998); or multiple antigen peptide systems (MAPs) (see e.g., Tam, J. P., Proc. Natl Acad. Sci. U.S.A. 85:5409-5413, 1988; Tam, J. P., J. Immunol. Methods 196: 17-32, 1996). In some embodiments, a cancer vaccine is formulated as a peptide-based vaccine, or nucleic acid based vaccine in which the nucleic acid encodes the polypeptides. In some embodiments, a cancer vaccine is formulated as an antibody-based vaccine. In some embodiments, a cancer vaccine is formulated as a cell based vaccine. In some embodiments, the cancer vaccine is a peptide cancer vaccine, which in some embodiments is a personalized peptide vaccine. In some embodiments, the cancer vaccine is a multivalent long peptide, a multiple peptide, a peptide mixture, a hybrid peptide, or a peptide pulsed dendritic cell vaccine (see, e.g., Yamada et al, Cancer Sci, 104: 14-21, 2013). In some embodiments, such cancer vaccines augment the anti-cancer response.

In some embodiments, the cancer vaccine comprises a polynucleotide that encodes a neoantigen, e.g., a neoantigen corresponding to a fusion nucleic acid molecule or polypeptide of the disclosure. In some embodiments, the cancer vaccine comprises DNA that encodes a neoantigen, e.g., a neoantigen corresponding to a fusion nucleic acid molecule or polypeptide of the disclosure. In some embodiments, the cancer vaccine comprises RNA that encodes a neoantigen, e.g., a neoantigen corresponding to a fusion nucleic acid molecule or polypeptide of the disclosure. In some embodiments, the cancer vaccine comprises a polynucleotide that encodes a neoantigen, e.g., a neoantigen corresponding to a fusion nucleic acid molecule or polypeptide of the disclosure. In some embodiments, the cancer vaccine further comprises one or more additional antigens, neoantigens, or other sequences that promote antigen presentation and/or an immune response. In some embodiments, the polynucleotide is complexed with one or more additional agents, such as a liposome or lipoplex. In some embodiments, the polynucleotide(s) are taken up and translated by antigen presenting cells (APCs), which then present the neoantigen(s) via MHC class I on the APC cell surface.

In some embodiments, the cancer vaccine is selected from sipuleucel-T (e.g., Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (e.g., Imlygic®, BioVex/Amgen, previously known as T-VEC), a genetically modified oncolytic viral therapy approved for treatment of unresectable cutaneous, subcutaneous and nodal lesions in melanoma. In some embodiments, the cancer vaccine is selected from an oncolytic viral therapy such as pexastimogene devacirepvec (PexaVec/JX-594, SillaJen/formerly Jennerex Biotherapeutics), a thymidine kinase-(TK-) deficient vaccinia virus engineered to express GM-CSF, for hepatocellular carcinoma (NCT02562755) and melanoma (NCT00429312); pelareorep (e.g., Reolysin®, Oncolytics Biotech), a variant of respiratory enteric orphan virus (reovirus) which does not replicate in cells that are not RAS-activated, in numerous cancers, including colorectal cancer (NCT01622543), prostate cancer (NCT01619813), head and neck squamous cell cancer (NCT01166542), pancreatic adenocarcinoma (NCT00998322), and non-small cell lung cancer (NSCLC) (NCT00861627); enadenotucirev (NG-348, PsiOxus, formerly known as ColoAdl), an adenovirus engineered to express a full length CD80 and an antibody fragment specific for the T-cell receptor CD3 protein, in ovarian cancer (NCT02028117), metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676), and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260), fallopian tube cancer, ovarian cancer (NCT02759588); or CG0070 (Cold Genesys), an adenovirus engineered to express GM-CSF in bladder cancer (NCT02365818); anti-gp100; STINGVAX; GVAX; DCVaxL; and DNX-2401. In some embodiments, the cancer vaccine is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TGO1 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFα-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8+ T cell response. In some embodiments, the cancer vaccine comprises a vector-based tumor antigen vaccine. Vector-based tumor antigen vaccines can be used as a way to provide a steady supply of antigens to stimulate an anti-tumor immune response. In some embodiments, vectors encoding for tumor antigens are injected into an individual (possibly with pro-inflammatory or other attractants such as GM-CSF), taken up by cells in vivo to make the specific antigens, which then provoke the desired immune response. In some embodiments, vectors may be used to deliver more than one tumor antigen at a time, to increase the immune response. In addition, recombinant virus, bacteria or yeast vectors can trigger their own immune responses, which may also enhance the overall immune response.

In some embodiments, the cancer vaccine comprises a DNA-based vaccine. In some embodiments, DNA-based vaccines can be employed to stimulate an anti-tumor response. The ability of directly injected DNA that encodes an antigenic protein, to elicit a protective immune response has been demonstrated in numerous experimental systems. Vaccination through directly injecting DNA that encodes an antigenic protein, to elicit a protective immune response often produces both cell-mediated and humoral responses. Moreover, reproducible immune responses to DNA encoding various antigens have been reported in mice that last essentially for the lifetime of the animal (see, e.g., Yankauckas et al. (1993) DNA Cell Biol., 12: 771-776). In some embodiments, plasmid (or other vector) DNA that includes a sequence encoding a protein operably linked to regulatory elements required for gene expression is administered to individuals (e.g. human patients, non-human mammals, etc.). In some embodiments, the cells of the individual take up the administered DNA and the coding sequence is expressed. In some embodiments, the antigen so produced becomes a target against which an immune response is directed.

In some embodiments, the cancer vaccine comprises an RNA-based vaccine. In some embodiments, RNA-based vaccines can be employed to stimulate an anti-tumor response. In some embodiments, RNA-based vaccines comprise a self-replicating RNA molecule. In some embodiments, the self-replicating RNA molecule may be an alphavirus-derived RNA replicon. Self-replicating RNA (or “SAM”) molecules are well known in the art and can be produced by using replication elements derived from, e.g., alphaviruses, and substituting the structural viral proteins with a nucleotide sequence encoding a protein of interest. A self-replicating RNA molecule is typically a +-strand molecule which can be directly translated after delivery to a cell, and this translation provides a RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded polypeptide, or may be transcribed to provide further transcripts with the same sense as the delivered RNA which are translated to provide in situ expression of the antigen.

In some embodiments, the cancer immunotherapy comprises a cell-based therapy. In some embodiments, the cancer immunotherapy comprises a T cell-based therapy. In some embodiments, the cancer immunotherapy comprises an adoptive therapy, e.g., an adoptive T cell-based therapy. In some embodiments, the T cells are autologous or allogeneic to the recipient. In some embodiments, the T cells are CD8+ T cells. In some embodiments, the T cells are CD4+ T cells. Adoptive immunotherapy refers to a therapeutic approach for treating cancer or infectious diseases in which immune cells are administered to a host with the aim that the cells mediate either directly or indirectly specific immunity to (i.e., mount an immune response directed against) cancer cells. In some embodiments, the immune response results in inhibition of tumor and/or metastatic cell growth and/or proliferation, and in related embodiments, results in neoplastic cell death and/or resorption. The immune cells can be derived from a different organism/host (exogenous immune cells) or can be cells obtained from the subject organism (autologous immune cells). In some embodiments, the immune cells (e.g., autologous or allogeneic T cells (e.g., regulatory T cells, CD4+ T cells, CD8+ T cells, or gamma-delta T cells), NK cells, invariant NK cells, or NKT cells) can be genetically engineered to express antigen receptors such as engineered TCRs and/or chimeric antigen receptors (CARs). For example, the host cells (e.g., autologous or allogeneic T-cells) are modified to express a T cell receptor (TCR) having antigenic specificity for a cancer antigen. In some embodiments, NK cells are engineered to express a TCR. The NK cells may be further engineered to express a CAR. Multiple CARs and/or TCRs, such as to different antigens, may be added to a single cell type, such as T cells or NK cells. In some embodiments, the cells comprise one or more nucleic acids/expression constructs/vectors introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g. chimeric). In some embodiments, a population of immune cells can be obtained from a subject in need of therapy or suffering from a disease associated with reduced immune cell activity. Thus, the cells will be autologous to the subject in need of therapy. In some embodiments, a population of immune cells can be obtained from a donor, such as a histocompatibility-matched donor. In some embodiments, the immune cell population can be harvested from the peripheral blood, cord blood, bone marrow, spleen, or any other organ/tissue in which immune cells reside in said subject or donor. In some embodiments, the immune cells can be isolated from a pool of subjects and/or donors, such as from pooled cord blood. In some embodiments, when the population of immune cells is obtained from a donor distinct from the subject, the donor may be allogeneic, provided the cells obtained are subject-compatible, in that they can be introduced into the subject. In some embodiments, allogeneic donor cells may or may not be human-leukocyte-antigen (HLA)-compatible. In some embodiments, to be rendered subject-compatible, allogeneic cells can be treated to reduce immunogenicity.

In some embodiments, the cell-based therapy comprises a T cell-based therapy, such as autologous cells, e.g., tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. Several approaches for the isolation, derivation, engineering or modification, activation, and expansion of functional anti-tumor effector cells have been described in the last two decades and may be used according to any of the methods provided herein. In some embodiments, the T cells are derived from the blood, bone marrow, lymph, umbilical cord, or lymphoid organs. In some embodiments, the cells are human cells. In some embodiments, the cells are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. In some embodiments, the cells may be allogeneic and/or autologous. In some embodiments, such as for off-the-shelf technologies, the cells are pluripotent and/or multipotent, such as stem cells, such as induced pluripotent stem cells (iPSCs).

In some embodiments, the T cell-based therapy comprises a chimeric antigen receptor (CAR)-T cell-based therapy. This approach involves engineering a CAR that specifically binds to an antigen of interest and comprises one or more intracellular signaling domains for T cell activation. The CAR is then expressed on the surface of engineered T cells (CAR-T) and administered to a patient, leading to a T-cell-specific immune response against cancer cells expressing the antigen. In some embodiments, the CAR specifically binds a neoantigen, such as a neoantigen corresponding to a fusion nucleic acid molecule or polypeptide of the disclosure

In some embodiments, the T cell-based therapy comprises T cells expressing a recombinant T cell receptor (TCR). This approach involves identifying a TCR that specifically binds to an antigen of interest, which is then used to replace the endogenous or native TCR on the surface of engineered T cells that are administered to a patient, leading to a T-cell-specific immune response against cancer cells expressing the antigen. In some embodiments, the recombinant TCR specifically binds a neoantigen corresponding to a fusion nucleic acid molecule or polypeptide of the disclosure.

In some embodiments, the T cell-based therapy comprises tumor-infiltrating lymphocytes (TILs). For example, TILs can be isolated from a tumor or cancer of the present disclosure, then isolated and expanded in vitro. Some or all of these TILs may specifically recognize an antigen expressed by the tumor or cancer of the present disclosure. In some embodiments, the TILs are exposed to one or more neoantigens, e.g., a neoantigen corresponding to a fusion nucleic acid molecule or polypeptide of the disclosure, e.g., a neoantigen, in vitro after isolation. TILs are then administered to the patient (optionally in combination with one or more cytokines or other immune-stimulating substances).

In some embodiments, the cell-based therapy comprises a natural killer (NK) cell-based therapy. Natural killer (NK) cells are a subpopulation of lymphocytes that have spontaneous cytotoxicity against a variety of tumor cells, virus-infected cells, and some normal cells in the bone marrow and thymus. NK cells are critical effectors of the early innate immune response toward transformed and virus-infected cells. NK cells can be detected by specific surface markers, such as CD16, CD56, and CD8 in humans. NK cells do not express T-cell antigen receptors, the pan T marker CD3, or surface immunoglobulin B cell receptors. In some embodiments, NK cells are derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well known in the art.

In some embodiments, the cell-based therapy comprises a dendritic cell (DC)-based therapy, e.g., a dendritic cell vaccine. In some embodiments, the DC vaccine comprises antigen-presenting cells that are able to induce specific T cell immunity, which are harvested from the patient or from a donor. In some embodiments, the DC vaccine can then be exposed in vitro to a peptide antigen, for which T cells are to be generated in the patient. In some embodiments, dendritic cells loaded with the antigen are then injected back into the patient. In some embodiments, immunization may be repeated multiple times if desired. Methods for harvesting, expanding, and administering dendritic cells are known in the art; see, e.g., WO2019178081. Dendritic cell vaccines (such as Sipuleucel-T, also known as APC8015 and PROVENGE®) are vaccines that involve administration of dendritic cells that act as APCs to present one or more cancer-specific antigens to the patient's immune system. In some embodiments, the dendritic cells are autologous or allogeneic to the recipient.

In some embodiments, the cancer immunotherapy comprises a TCR-based therapy. In some embodiments, the cancer immunotherapy comprises administration of one or more TCRs or TCR-based therapeutics that specifically bind an antigen expressed by a cancer of the present disclosure, e.g., a neoantigen corresponding to a fusion nucleic acid molecule or polypeptide of the disclosure. In some embodiments, the TCR-based therapeutic may further include a moiety that binds an immune cell (e.g., a T cell), such as an antibody or antibody fragment that specifically binds a T cell surface protein or receptor (e.g., an anti-CD3 antibody or antibody fragment).

In some embodiments, the immunotherapy comprises adjuvant immunotherapy. Adjuvant immunotherapy comprises the use of one or more agents that activate components of the innate immune system, e.g., HILTONOL® (imiquimod), which targets the TLR7 pathway.

In some embodiments, the immunotherapy comprises cytokine immunotherapy. Cytokine immunotherapy comprises the use of one or more cytokines that activate components of the immune system. Examples include, but are not limited to, aldesleukin (e.g., PROLEUKIN®; interleukin-2), interferon alfa-2a (e.g., ROFERON®-A), interferon alfa-2b (e.g., INTRON®-A), and peginterferon alfa-2b (e.g., PEGINTRON®).

In some embodiments, the immunotherapy comprises oncolytic virus therapy. Oncolytic virus therapy uses genetically modified viruses to replicate in and kill cancer cells, leading to the release of antigens that stimulate an immune response. In some embodiments, replication-competent oncolytic viruses expressing a tumor antigen comprise any naturally occurring (e.g., from a “field source”) or modified replication-competent oncolytic virus. In some embodiments, the oncolytic virus, in addition to expressing a tumor antigen, may be modified to increase selectivity of the virus for cancer cells. In some embodiments, replication-competent oncolytic viruses include, but are not limited to, oncolytic viruses that are a member in the family of myoviridae, siphoviridae, podpviridae, teciviridae, corticoviridae, plasmaviridae, lipothrixviridae, fuselloviridae, poxyiridae, iridoviridae, phycodnaviridae, baculoviridae, herpesviridae, adnoviridae, papovaviridae, polydnaviridae, inoviridae, microviridae, geminiviridae, circoviridae, parvoviridae, hcpadnaviridae, retroviridae, cyctoviridae, reoviridae, birnaviridae, paramyxoviridae, rhabdoviridae, filoviridae, orthomyxoviridae, bunyaviridae, arenaviridae, Leviviridae, picornaviridae, sequiviridae, comoviridae, potyviridae, caliciviridae, astroviridae, nodaviridae, tetraviridae, tombusviridae, coronaviridae, glaviviridae, togaviridae, and barnaviridae. In some embodiments, replication-competent oncolytic viruses include adenovirus, retrovirus, reovirus, rhabdovirus, Newcastle Disease virus (NDV), polyoma virus, vaccinia virus (VacV), herpes simplex virus, picornavirus, coxsackie virus and parvovirus. In some embodiments, a replicative oncolytic vaccinia virus expressing a tumor antigen may be engineered to lack one or more functional genes in order to increase the cancer selectivity of the virus. In some embodiments, an oncolytic vaccinia virus is engineered to lack thymidine kinase (TK) activity. In some embodiments, the oncolytic vaccinia virus may be engineered to lack vaccinia virus growth factor (VGF). In some embodiments, an oncolytic vaccinia virus may be engineered to lack both VGF and TK activity. In some embodiments, an oncolytic vaccinia virus may be engineered to lack one or more genes involved in evading host interferon (IFN) response such as E3L, K3L, B18R, or B8R. In some embodiments, a replicative oncolytic vaccinia virus is a Western Reserve, Copenhagen, Lister or Wyeth strain and lacks a functional TK gene. In some embodiments, the oncolytic vaccinia virus is a Western Reserve, Copenhagen, Lister or Wyeth strain lacking a functional B18R and/or B8R gene. In some embodiments, a replicative oncolytic vaccinia virus expressing a tumor antigen may be locally or systemically administered to a subject, e.g. via intratumoral, intraperitoneal, intravenous, intra-arterial, intramuscular, intradermal, intracranial, subcutaneous, or intranasal administration.

In some embodiments, the anti-cancer therapy comprises an immune checkpoint inhibitor, e.g., alone or in combination with an ALK-, BRAF-, EGFR-, ERBB2-, FGFR1-, FGFR2-, FGFR3-, MET-, NTRK1-, RAF1-, RET-, or ROS1-targeted therapy. In some embodiments, the methods provided herein comprise administering to an individual an effective amount of an immune checkpoint inhibitor. As is known in the art, a checkpoint inhibitor targets at least one immune checkpoint protein to alter the regulation of an immune response. Immune checkpoint proteins include, e.g., CTLA4, PD-L1, PD-1, PD-L2, VISTA, B7-H2, B7-H3, B7-H4, B7-H6, 2B4, ICOS, HVEM, CEACAM, LAIR1, CD80, CD86, CD276, VTCN1, MHC class I, MHC class II, GALS, adenosine, TGFR, CSF1R, MICA/B, arginase, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, LAG-3, BTLA, IDO, OX40, and A2aR. In some embodiments, molecules involved in regulating immune checkpoints include, but are not limited to: PD-1 (CD279), PD-L1 (B7-H1, CD274), PD-L2 (B7-CD, CD273), CTLA-4 (CD152), HVEM, BTLA (CD272), a killer-cell immunoglobulin-like receptor (KIR), LAG-3 (CD223), TIM-3 (HAVCR2), CEACAM, CEACAM-1, CEACAM-3, CEACAM-5, GAL9, VISTA (PD-1H), TIGIT, LAIR1, CD160, 2B4, TGFRbeta, A2AR, GITR (CD357), CD80 (B7-1), CD86 (B7-2), CD276 (B7-H3), VTCN1 (B7-H4), MHC class I, MHC class II, GALS, adenosine, TGFR, B7-H1, OX40 (CD134), CD94 (KLRD1), CD137 (4-1BB), CD137L (4-1BBL), CD40, IDO, CSF1R, CD40L, CD47, CD70 (CD27L), CD226, HHLA2, ICOS (CD278), ICOSL (CD275), LIGHT (TNFSF14, CD258), NKG2a, NKG2d, OX40L (CD134L), PVR (NECL5, CD155), SIRPa, MICA/B, and/or arginase. In some embodiments, an immune checkpoint inhibitor (i.e., a checkpoint inhibitor) decreases the activity of a checkpoint protein that negatively regulates immune cell function, e.g., in order to enhance T cell activation and/or an anti-cancer immune response. In other embodiments, a checkpoint inhibitor increases the activity of a checkpoint protein that positively regulates immune cell function, e.g., in order to enhance T cell activation and/or an anti-cancer immune response. In some embodiments, the checkpoint inhibitor is an antibody. Examples of checkpoint inhibitors include, without limitation, a PD-1 axis binding antagonist, a PD-L1 axis binding antagonist (e.g., an anti-PD-L1 antibody, e.g., atezolizumab (MPDL3280A)), an antagonist directed against a co-inhibitory molecule (e.g., a CTLA4 antagonist (e.g., an anti-CTLA4 antibody), a TIM-3 antagonist (e.g., an anti-TIM-3 antibody), or a LAG-3 antagonist (e.g., an anti-LAG-3 antibody)), or any combination thereof. In some embodiments, the immune checkpoint inhibitors comprise drugs such as small molecules, recombinant forms of ligand or receptors, or antibodies, such as human antibodies (see, e.g., International Patent Publication WO2015016718; Pardoll, Nat Rev Cancer, 12(4): 252-64, 2012; both incorporated herein by reference). In some embodiments, known inhibitors of immune checkpoint proteins or analogs thereof may be used, in particular chimerized, humanized or human forms of antibodies may be used.

In some embodiments, the checkpoint inhibitor is a PD-L1 axis binding antagonist. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. PD-L1 (programmed death ligand 1) is also referred to in the art as “programmed cell death 1 ligand 1,” “PDCD1 LG1,” “CD274,” “B7-H,” and “PDL1.” An exemplary human PD-L1 is shown in UniProtKB/Swiss-Prot Accession No.Q9NZQ7.1. PD-L2 (programmed death ligand 2) is also referred to in the art as “programmed cell death 1 ligand 2,” “PDCD1 LG2,” “CD273,” “B7-DC,” “Btdc,” and “PDL2.” An exemplary human PD-L2 is shown in UniProtKB/Swiss-Prot Accession No. Q9BQ51. In some instances, PD-1, PD-L1, and PD-L2 are human PD-1, PD-L1 and PD-L2.

In some instances, the PD-1 binding antagonist is a molecule that inhibits the binding of PD-1 to its ligand binding partners. In a specific embodiment, the PD-1 ligand binding partners are PD-L1 and/or PD-L2. In another instance, a PD-L1 binding antagonist is a molecule that inhibits the binding of PD-L1 to its binding ligands. In a specific embodiment, PD-L1 binding partners are PD-1 and/or B7-1. In another instance, the PD-L2 binding antagonist is a molecule that inhibits the binding of PD-L2 to its ligand binding partners. In a specific embodiment, the PD-L2 binding ligand partner is PD-1. The antagonist may be an antibody, an antigen binding fragment thereof, an immunoadhesin, a fusion protein, or an oligopeptide. In some embodiments, the PD-1 binding antagonist is a small molecule, a nucleic acid, a polypeptide (e.g., antibody), a carbohydrate, a lipid, a metal, or a toxin.

In some instances, the PD-1 binding antagonist is an anti-PD-1 antibody (e.g., a human antibody, a humanized antibody, or a chimeric antibody), for example, as described below. In some instances, the anti-PD-1 antibody is one or more of MDX-1 106 (nivolumab), MK-3475 (pembrolizumab, e.g., Keytruda®), MEDI-0680 (AMP-514), PDR001, REGN2810, MGA-012, JNJ-63723283, BI 754091, or BGB-108. In other instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some instances, the PD-1 binding antagonist is AMP-224. Other examples of anti-PD-1 antibodies include, but are not limited to, MEDI-0680 (AMP-514; AstraZeneca), PDR001 (CAS Registry No. 1859072-53-9; Novartis), REGN2810 (e.g., LIBTAYO® or cemiplimab-rwlc; Regeneron), BGB-108 (BeiGene), BGB-A317 (BeiGene), BI 754091, JS-001 (Shanghai Junshi), STI-A1110 (Sorrento), INCSHR-1210 (Incyte), PF-06801591 (Pfizer), TSR-042 (also known as ANBO11; Tesaro/AnaptysBio), AM0001 (ARMO Biosciences), ENUM 244C8 (Enumeral Biomedical Holdings), or ENUM 388D4 (Enumeral Biomedical Holdings). In some embodiments, the PD-1 axis binding antagonist comprises tislelizumab (BGB-A317), BGB-108, STI-A1110, AM0001, BI 754091, sintilimab (IBI308), cetrelimab (JNJ-63723283), toripalimab (JS-001), camrelizumab (SHR-1210, INCSHR-1210, HR-301210), MEDI-0680 (AMP-514), MGA-012 (INCMGA 0012), nivolumab (BMS-936558, MDX1106, ONO-4538), spartalizumab (PDR001), pembrolizumab (MK-3475, SCH 900475, e.g., Keytruda®), PF-06801591, cemiplimab (REGN-2810, REGEN2810), dostarlimab (TSR-042, ANBO11), FITC-YT-16 (PD-1 binding peptide), APL-501 or CBT-501 or genolimzumab (GB-226), AB-122, AK105, AMG 404, BCD-100, F520, HLX10, HX008, JTX-4014, LZM009, Sym021, PSB205, AMP-224 (fusion protein targeting PD-1), CX-188 (PD-1 probody), AGEN-2034, GLS-010, budigalimab (ABBV-181), AK-103, BAT-1306, CS-1003, AM-0001, TILT-123, BH-2922, BH-2941, BH-2950, ENUM-244C8, ENUM-388D4, HAB-21, H EISCOI 11-003, IKT-202, MCLA-134, MT-17000, PEGMP-7, PRS-332, RXI-762, STI-1110, VXM-10, XmAb-23104, AK-112, HLX-20, SSI-361, AT-16201, SNA-01, AB122, PD1-PIK, PF-06936308, RG-7769, CAB PD-1 Abs, AK-123, MEDI-3387, MEDI-5771, 4H1128Z-E27, REMD-288, SG-001, BY-24.3, CB-201, IBI-319, ONCR-177, Max-1, CS-4100, JBI-426, CCC-0701, or CCX-4503, or derivatives thereof.

In some embodiments, the PD-L1 binding antagonist is a small molecule that inhibits PD-1. In some embodiments, the PD-L1 binding antagonist is a small molecule that inhibits PD-L1. In some embodiments, the PD-L1 binding antagonist is a small molecule that inhibits PD-L1 and VISTA or PD-L1 and TIM3. In some embodiments, the PD-L1 binding antagonist is CA-170 (also known as AUPM-170). In some embodiments, the PD-L1 binding antagonist is an anti-PD-L1 antibody. In some embodiments, the anti-PD-L1 antibody can bind to a human PD-L1, for example a human PD-L1 as shown in UniProtKB/Swiss-Prot Accession No.Q9NZQ7.1, or a variant thereof. In some embodiments, the PD-L1 binding antagonist is a small molecule, a nucleic acid, a polypeptide (e.g., antibody), a carbohydrate, a lipid, a metal, or a toxin.

In some instances, the PD-L1 binding antagonist is an anti-PD-L1 antibody, for example, as described below. In some instances, the anti-PD-L1 antibody is capable of inhibiting the binding between PD-L1 and PD-1, and/or between PD-L1 and B7-1. In some instances, the anti-PD-L1 antibody is a monoclonal antibody. In some instances, the anti-PD-L1 antibody is an antibody fragment selected from a Fab, Fab′-SH, Fv, scFv, or (Fab′)2 fragment. In some instances, the anti-PD-L1 antibody is a humanized antibody. In some instances, the anti-PD-L1 antibody is a human antibody. In some instances, the anti-PD-L1 antibody is selected from YW243.55.S70, MPDL3280A (atezolizumab), MDX-1 105, MED14736 (durvalumab), or MSB0010718C (avelumab). In some embodiments, the PD-L1 axis binding antagonist comprises atezolizumab, avelumab, durvalumab (imfinzi), BGB-A333, SHR-1316 (HTI-1088), CK-301, BMS-936559, envafolimab (KN035, ASC22), CS1001, MDX-1105 (BMS-936559), LY3300054, STI-A1014, FAZ053, CX-072, INCB086550, GNS-1480, CA-170, CK-301, M-7824, HTI-1088 (HTI-131, SHR-1316), MSB-2311, AK-106, AVA-004, BBI-801, CA-327, CBA-0710, CBT-502, FPT-155, IKT-201, IKT-703, 10-103, JS-003, KD-033, KY-1003, MCLA-145, MT-5050, SNA-02, BCD-135, APL-502 (CBT-402 or TQB2450), IMC-001, KD-045, INBRX-105, KN-046, IMC-2102, IMC-2101, KD-005, IMM-2502, 89Zr-CX-072, 89Zr-DFO-6E11, KY-1055, MEDI-1109, MT-5594, SL-279252, DSP-106, Gensci-047, REMD-290, N-809, PRS-344, FS-222, GEN-1046, BH-29xx, or FS-118, or a derivative thereof.

In some embodiments, the checkpoint inhibitor is an antagonist of CTLA4. In some embodiments, the checkpoint inhibitor is a small molecule antagonist of CTLA4. In some embodiments, the checkpoint inhibitor is an anti-CTLA4 antibody. CTLA4 is part of the CD28-B7 immunoglobulin superfamily of immune checkpoint molecules that acts to negatively regulate T cell activation, particularly CD28-dependent T cell responses. CTLA4 competes for binding to common ligands with CD28, such as CD80 (B7-1) and CD86 (B7-2), and binds to these ligands with higher affinity than CD28. Blocking CTLA4 activity (e.g., using an anti-CTLA4 antibody) is thought to enhance CD28-mediated costimulation (leading to increased T cell activation/priming), affect T cell development, and/or deplete Tregs (such as intratumoral Tregs). In some embodiments, the CTLA4 antagonist is a small molecule, a nucleic acid, a polypeptide (e.g., antibody), a carbohydrate, a lipid, a metal, or a toxin. In some embodiments, the CTLA-4 inhibitor comprises ipilimumab (IBI310, BMS-734016, MDX010, MDX-CTLA4, MED14736), tremelimumab (CP-675, CP-675,206), APL-509, AGEN1884, CS1002, AGEN1181, Abatacept (Orencia, BMS-188667, RG2077), BCD-145, ONC-392, ADU-1604, REGN4659, ADG116, KN044, KN046, or a derivative thereof.

In some embodiments, the anti-PD-1 antibody or antibody fragment is MDX-1106 (nivolumab), MK-3475 (pembrolizumab, e.g., Keytruda®), MEDI-0680 (AMP-514), PDR001, REGN2810, MGA-012, JNJ-63723283, BI 754091, BGB-108, BGB-A317, JS-001, STI-A1110, INCSHR-1210, PF-06801591, TSR-042, AM0001, ENUM 244C8, or ENUM 388D4. In some embodiments, the PD-1 binding antagonist is an anti-PD-1 immunoadhesin. In some embodiments, the anti-PD-1 immunoadhesin is AMP-224. In some embodiments, the anti-PD-L1 antibody or antibody fragment is YW243.55.S70, MPDL3280A (atezolizumab), MDX-1105, MEDI4736 (durvalumab), MSB0010718C (avelumab), LY3300054, STI-A1014, KN035, FAZ053, or CX-072.

In some embodiments, the immune checkpoint inhibitor comprises a LAG-3 inhibitor (e.g., an antibody, an antibody conjugate, or an antigen-binding fragment thereof). In some embodiments, the LAG-3 inhibitor comprises a small molecule, a nucleic acid, a polypeptide (e.g., an antibody), a carbohydrate, a lipid, a metal, or a toxin. In some embodiments, the LAG-3 inhibitor comprises a small molecule. In some embodiments, the LAG-3 inhibitor comprises a LAG-3 binding agent. In some embodiments, the LAG-3 inhibitor comprises an antibody, an antibody conjugate, or an antigen-binding fragment thereof. In some embodiments, the LAG-3 inhibitor comprises eftilagimod alpha (IMP321, IMP-321, EDDP-202, EOC-202), relatlimab (BMS-986016), GSK2831781 (IMP-731), LAG525 (IMP701), TSR-033, EVIP321 (soluble LAG-3 protein), BI 754111, IMP761, REGN3767, MK-4280, MGD-013, XmAb22841, INCAGN-2385, ENUM-006, AVA-017, AM-0003, iOnctura anti-LAG-3 antibody, Arcus Biosciences LAG-3 antibody, Sym022, a derivative thereof, or an antibody that competes with any of the preceding.

In some embodiments, the anti-cancer therapy comprises an immunoregulatory molecule or a cytokine, e.g., alone or in combination with an ALK-, BRAF-, EGFR-, ERBB2-, FGFR1-, FGFR2-, FGFR3-, MET-, NTRK1-, RAF1-, RET-, or ROS1-targeted therapy. An immunoregulatory profile is required to trigger an efficient immune response and balance the immunity in a subject. Examples of suitable immunoregulatory cytokines include, but are not limited to, interferons (e.g., IFNα, IFNβ and IFNγ), interleukins (e.g., IL-1, IL-2, IL-3, IL-4, IL-5, IL-6, IL-7, IL-8, IL-9, IL-10, IL-12 and IL-20), tumor necrosis factors (e.g., TNFα and TNFβ), erythropoietin (EPO), FLT-3 ligand, gIp10, TCA-3, MCP-1, MIF, MIP-1α, MIP-10, Rantes, macrophage colony stimulating factor (M-CSF), granulocyte colony stimulating factor (G-CSF), or granulocyte-macrophage colony stimulating factor (GM-CSF), as well as functional fragments thereof. In some embodiments, any immunomodulatory chemokine that binds to a chemokine receptor, i.e., a CXC, CC, C, or CX3C chemokine receptor, can be used in the context of the present disclosure. Examples of chemokines include, but are not limited to, MIP-3a (Lax), MIP-3β, Hcc-1, MPIF-1, MPIF-2, MCP-2, MCP-3, MCP-4, MCP-5, Eotaxin, Tarc, Elc, I309, IL-8, GCP-2 Groα, Gro-β, Nap-2, Ena-78, Ip-10, MIG, I-Tac, SDF-1, or BCA-1 (Blc), as well as functional fragments thereof. In some embodiments, the immunoregulatory molecule is included with any of the treatments provided herein.

In some embodiments, the immune checkpoint inhibitor is monovalent and/or monospecific. In some embodiments, the immune checkpoint inhibitor is multivalent and/or multispecific.

In some embodiments, the anti-cancer therapy comprises an anti-cancer agent that inhibits expression of a nucleic acid that comprises or encodes a fusion nucleic acid molecule of the disclosure or a portion thereof, or a fusion polypeptide of the disclosure, or a portion thereof. In some embodiments, the anti-cancer therapy comprises a nucleic acid molecule, such as a dsRNA, an siRNA, or an shRNA. As is known in the art, dsRNAs having a duplex structure are effective at inducing RNA interference (RNAi). In some embodiments, the anti-cancer therapy comprises a small interfering RNA molecule (siRNA). dsRNAs and siRNAs can be used to silence gene expression in mammalian cells (e.g., human cells). In some embodiments, a dsRNA of the disclosure comprises any of between about 5 and about 10 base pairs, between about 10 and about 12 base pairs, between about 12 and about 15 base pairs, between about 15 and about 20 base pairs, between about 20 and 23 base pairs, between about 23 and about 25 base pairs, between about 25 and about 27 base pairs, or between about 27 and about 30 base pairs. As is known in the art, siRNAs are small dsRNAs that optionally include overhangs. In some embodiments, the duplex region of an siRNA is between about 18 and 25 nucleotides, e.g., any of 18, 19, 20, 21, 22, 23, 24, or 25 nucleotides. siRNAs may also include short hairpin RNAs (shRNAs), e.g., with approximately 29-base-pair stems and 2-nucleotide 3′ overhangs. In some embodiments, a dsRNA, an siRNA, or an shRNA of the disclosure comprises a nucleotide sequence that is configured to hybridize to a nucleic acid that comprises or encodes a fusion nucleic acid molecule of the disclosure or a portion thereof comprising a breakpoint. Methods for designing, optimizing, producing, and using dsRNAs, siRNAs, or shRNAs, are known in the art.

In some embodiments, the anti-cancer therapy comprises a chemotherapy, e.g., alone or in combination with an ALK-, BRAF-, EGFR-, ERBB2-, FGFR1-, FGFR2-, FGFR3-, MET-, NTRK1-, RAF1-, RET-, or ROS1-targeted therapy. Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antibiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenishers such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylomithine (DMFO); retinoids, such as retinoic acid; capecitabine; carboplatin, procarbazine, plicomycin, gemcitabine, navelbine, famesyl-protein tansferase inhibitors, transplatinum, and pharmaceutically acceptable salts, acids, or derivatives of any of the above.

Some non-limiting examples of chemotherapeutic drugs which can be combined with anti-cancer therapies of the present disclosure are carboplatin (Paraplatin), cisplatin (Platinol, Platinol-AQ), cyclophosphamide (Cytoxan, Neosar), docetaxel (Taxotere), doxorubicin (Adriamycin), erlotinib (Tarceva), etoposide (VePesid), fluorouracil (5-FU), gemcitabine (Gemzar), imatinib mesylate (Gleevec), irinotecan (Camptosar), methotrexate (Folex, Mexate, Amethopterin), paclitaxel (Taxol, Abraxane), sorafinib (Nexavar), sunitinib (Sutent), topotecan (Hycamtin), vincristine (Oncovin, Vincasar PFS), and vinblastine (Velban).

In some embodiments, the anti-cancer therapy comprises a kinase inhibitor, e.g., alone or in combination with an ALK-, BRAF-, EGFR-, ERBB2-, FGFR1-, FGFR2-, FGFR3-, MET-, NTRK1-, RAF1-, RET-, or ROS1-targeted therapy. Examples of kinase inhibitors include those that target one or more receptor tyrosine kinases, e.g., BCR-ABL, B-Raf, EGFR, HER-2/ErbB2, IGF-IR, PDGFR-a, PDGFR-β, cKit, Flt-4, Flt3, FGFR1, FGFR2, FGFR3, FGFR4, CSF1R, c-Met, ROS1, RON, c-Ret, or ALK; one or more cytoplasmic tyrosine kinases, e.g., c-SRC, c-YES, Ab1, or JAK-2; one or more serine/threonine kinases, e.g., ATM, Aurora A & B, CDKs, mTOR, PKCi, PLKs, b-Raf, c-Raf, S6K, or STK11/LKB1; or one or more lipid kinases, e.g., PI3K or SKI. Small molecule kinase inhibitors include PHA-739358, nilotinib, dasatinib, PD166326, NSC 743411, lapatinib (GW-572016), canertinib (CI-1033), semaxinib (SU5416), vatalanib (PTK787/ZK222584), sutent (SU1 1248), sorafenib (BAY 43-9006), or leflunomide (SU101). Additional non-limiting examples of tyrosine kinase inhibitors include imatinib (Gleevec/Glivec) and gefitinib (Iressa).

In some embodiments, the anti-cancer therapy comprises an anti-angiogenic agent, e.g., alone or in combination with an ALK-, BRAF-, EGFR-, ERBB2-, FGFR1-, FGFR2-, FGFR3-, MET-, NTRK1-, RAF1-, RET-, or ROS1-targeted therapy. Angiogenesis inhibitors prevent the extensive growth of blood vessels (angiogenesis) that tumors require to survive. Non-limiting examples of angiogenesis-mediating molecules or angiogenesis inhibitors which may be used in the methods of the present disclosure include soluble VEGF (for example: VEGF isoforms, e.g., VEGF121 and VEGF165; VEGF receptors, e.g., VEGFR1, VEGFR2; and co-receptors, e.g., Neuropilin-1 and Neuropilin-2), NRP-1, angiopoietin 2, TSP-1 and TSP-2, angiostatin and related molecules, endostatin, vasostatin, calreticulin, platelet factor-4, TIMP and CDAI, Meth-1 and Meth-2, IFNα, IFN-β and IFN-γ, CXCL10, IL-4, IL-12 and IL-18, prothrombin (kringle domain-2), antithrombin III fragment, prolactin, VEGI, SPARC, osteopontin, maspin, canstatin, proliferin-related protein, restin and drugs such as bevacizumab, itraconazole, carboxyamidotriazole, TNP-470, CM101, IFN-a platelet factor-4, suramin, SU5416, thrombospondin, VEGFR antagonists, angiostatic steroids and heparin, cartilage-derived angiogenesis inhibitory factor, matrix metalloproteinase inhibitors, 2-methoxyestradiol, tecogalan, tetrathiomolybdate, thalidomide, thrombospondin, prolactina v β3 inhibitors, linomide, or tasquinimod. In some embodiments, known therapeutic candidates that may be used according to the methods of the disclosure include naturally occurring angiogenic inhibitors, including without limitation, angiostatin, endostatin, or platelet factor-4. In another embodiment, therapeutic candidates that may be used according to the methods of the disclosure include, without limitation, specific inhibitors of endothelial cell growth, such as TNP-470, thalidomide, and interleukin-12. Still other anti-angiogenic agents that may be used according to the methods of the disclosure include those that neutralize angiogenic molecules, including without limitation, antibodies to fibroblast growth factor, antibodies to vascular endothelial growth factor, antibodies to platelet derived growth factor, or antibodies or other types of inhibitors of the receptors of EGF, VEGF or PDGF. In some embodiments, anti-angiogenic agents that may be used according to the methods of the disclosure include, without limitation, suramin and its analogs, and tecogalan. In other embodiments, anti-angiogenic agents that may be used according to the methods of the disclosure include, without limitation, agents that neutralize receptors for angiogenic factors or agents that interfere with vascular basement membrane and extracellular matrix, including, without limitation, metalloprotease inhibitors and angiostatic steroids. Another group of anti-angiogenic compounds that may be used according to the methods of the disclosure includes, without limitation, anti-adhesion molecules, such as antibodies to integrin alpha v beta 3. Still other anti-angiogenic compounds or compositions that may be used according to the methods of the disclosure include, without limitation, kinase inhibitors, thalidomide, itraconazole, carboxyamidotriazole, CM101, IFN-α, IL-12, SU5416, thrombospondin, cartilage-derived angiogenesis inhibitory factor, 2-methoxyestradiol, tetrathiomolybdate, thrombospondin, prolactin, and linomide. In one particular embodiment, the anti-angiogenic compound that may be used according to the methods of the disclosure is an antibody to VEGF, such as Avastin®/bevacizumab (Genentech).

In some embodiments, the anti-cancer therapy comprises an anti-DNA repair therapy, e.g., alone or in combination with an ALK-, BRAF-, EGFR-, ERBB2-, FGFR1-, FGFR2-, FGFR3-, MET-, NTRK1-, RAF1-, RET-, or ROS1-targeted therapy. In some embodiments, the anti-DNA repair therapy is a PARP inhibitor (e.g., talazoparib, rucaparib, olaparib), a RAD51 inhibitor (e.g., RI-1), or an inhibitor of a DNA damage response kinase, e.g., CHCK1 (e.g., AZD7762), ATM (e.g., KU-55933, KU-60019, NU7026, or VE-821), and ATR (e.g., NU7026).

In some embodiments, the anti-cancer therapy comprises a radiosensitizer, e.g., alone or in combination with an ALK-, BRAF-, EGFR-, ERBB2-, FGFR1-, FGFR2-, FGFR3-, MET-, NTRK1-, RAF1-, RET-, or ROS1-targeted therapy. Exemplary radiosensitizers include hypoxia radiosensitizers such as misonidazole, metronidazole, and trans-sodium crocetinate, a compound that helps to increase the diffusion of oxygen into hypoxic tumor tissue. The radiosensitizer can also be a DNA damage response inhibitor interfering with base excision repair (BER), nucleotide excision repair (NER), mismatch repair (MMR), recombinational repair comprising homologous recombination (HR) and non-homologous end-joining (NHEJ), and direct repair mechanisms. Single strand break (SSB) repair mechanisms include BER, NER, or MMR pathways, while double stranded break (DSB) repair mechanisms consist of HR and NHEJ pathways. Radiation causes DNA breaks that, if not repaired, are lethal. SSBs are repaired through a combination of BER, NER and MMR mechanisms using the intact DNA strand as a template. The predominant pathway of SSB repair is BER, utilizing a family of related enzymes termed poly-(ADP-ribose) polymerases (PARP). Thus, the radiosensitizer can include DNA damage response inhibitors such as PARP inhibitors.

In some embodiments, the anti-cancer therapy comprises an anti-inflammatory agent, e.g., alone or in combination with an ALK-, BRAF-, EGFR-, ERBB2-, FGFR1-, FGFR2-, FGFR3-, MET-, NTRK1-, RAF1-, RET-, or ROS1-targeted therapy. In some embodiments, the anti-inflammatory agent is an agent that blocks, inhibits, or reduces inflammation or signaling from an inflammatory signaling pathway In some embodiments, the anti-inflammatory agent inhibits or reduces the activity of one or more of any of the following: IL-1, IL-2, IL-3, IL-4, IL-5, IL-6, IL-7, IL-8, IL-9, IL-10, IL-12, IL-13, IL-15, IL-18, IL-23; interferons (IFNs), e.g., IFNα, IFNβ, IFNγ, IFN-γ inducing factor (IGIF); transforming growth factor-β (TGF-β); transforming growth factor-α (TGF-α); tumor necrosis factors, e.g., TNF-α, TNF-β, TNF-RI, TNF-RII; CD23; CD30; CD40L; EGF; G-CSF; GDNF; PDGF-BB; RANTES/CCL5; IKK; NF-κB; TLR2; TLR3; TLR4; TL5; TLR6; TLR7; TLR8; TLR8; TLR9; and/or any cognate receptors thereof. In some embodiments, the anti-inflammatory agent is an IL-1 or IL-1 receptor antagonist, such as anakinra (e.g., Kineret®), rilonacept, or canakinumab. In some embodiments, the anti-inflammatory agent is an IL-6 or IL-6 receptor antagonist, e.g., an anti-IL-6 antibody or an anti-IL-6 receptor antibody, such as tocilizumab (e.g., ACTEMRA®), olokizumab, clazakizumab, sarilumab, sirukumab, siltuximab, or ALX-0061. In some embodiments, the anti-inflammatory agent is a TNF-α antagonist, e.g., an anti-TNFα antibody, such as infliximab (Remicade®), golimumab (Simponi®), adalimumab (e.g., Humira®), certolizumab pegol (e.g., Cimzia®) or etanercept. In some embodiments, the anti-inflammatory agent is a corticosteroid. Exemplary corticosteroids include, but are not limited to, cortisone (hydrocortisone, hydrocortisone sodium phosphate, hydrocortisone sodium succinate, e.g., Ala-Cort®, Hydrocort Acetate®, hydrocortone phosphate Lanacort®, Solu-Cortef®), decadron (dexamethasone, dexamethasone acetate, dexamethasone sodium phosphate, e.g., Dexasone®, Diodex®, Hexadrol®, Maxidex®), methylprednisolone (6-methylprednisolone, methylprednisolone acetate, methylprednisolone sodium succinate, e.g., Duralone®, Medralone®, Medrol®, M-Prednisol®, Solu-Medrol®), prednisolone (e.g., Delta-Cortef®, ORAPRED®, Pediapred®, Prezone®), and prednisone (e.g., Deltasone®, Liquid Pred®, Meticorten®, Orasone®), and bisphosphonates (e.g., pamidronate (Aredia®), and zoledronic acid (e.g., Zometac®).

In some embodiments, the anti-cancer therapy comprises an anti-hormonal agent, e.g., alone or in combination with an ALK-, BRAF-, EGFR-, ERBB2-, FGFR1-, FGFR2-, FGFR3-, MET-, NTRK1-, RAF1-, RET-, or ROS1-targeted therapy. Anti-hormonal agents are agents that act to regulate or inhibit hormone action on tumors. Examples of anti-hormonal agents include anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4(5)-imidazoles, aminoglutethimide, MEGACE® megestrol acetate, AROMASIN® exemestane, formestanie, fadrozole, RIVISOR® vorozole, FEMARA® letrozole, and ARIMIDEX® (anastrozole); anti-androgens such as flutamide, nilutamide, bicalutamide, leuprolide, and goserelin; troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

In some embodiments, the anti-cancer therapy comprises an antimetabolite chemotherapeutic agent, e.g., alone or in combination with an ALK-, BRAF-, EGFR-, ERBB2-, FGFR1-, FGFR2-, FGFR3-, MET-, NTRK1-, RAF1-, RET-, or ROS1-targeted therapy. Antimetabolite chemotherapeutic agents are agents that are structurally similar to a metabolite, but cannot be used by the body in a productive manner. Many antimetabolite chemotherapeutic agents interfere with the production of RNA or DNA. Examples of antimetabolite chemotherapeutic agents include gemcitabine (e.g., GEMZAR®), 5-fluorouracil (5-FU), capecitabine (e.g., XELODA™), 6-mercaptopurine, methotrexate, 6-thioguanine, pemetrexed, raltitrexed, arabinosylcytosine ARA-C cytarabine (e.g., CYTOSAR-U®), dacarbazine (DTIC-DOMED), azocytosine, deoxycytosine, pyridmidene, fludarabine (e.g., FLUDARA®), cladrabine, and 2-deoxy-D-glucose. In some embodiments, an antimetabolite chemotherapeutic agent is gemcitabine. Gemcitabine HCl is sold by Eli Lilly under the trademark GEMZAR®.

In some embodiments, the anti-cancer therapy comprises a platinum-based chemotherapeutic agent, e.g., alone or in combination with an ALK-, BRAF-, EGFR-, ERBB2-, FGFR1-, FGFR2-, FGFR3-, MET-, NTRK1-, RAF1-, RET-, or ROS1-targeted therapy. Platinum-based chemotherapeutic agents are chemotherapeutic agents that comprise an organic compound containing platinum as an integral part of the molecule. In some embodiments, a chemotherapeutic agent is a platinum agent. In some such embodiments, the platinum agent is selected from cisplatin, carboplatin, oxaliplatin, nedaplatin, triplatin tetranitrate, phenanthriplatin, picoplatin, or satraplatin.

In some aspects, provided herein are therapeutic formulations comprising an anti-cancer therapy provided herein, and a pharmaceutically acceptable carrier, excipient, or stabilizer. A formulation provided herein may contain more than one active compound, e.g., an anti-cancer therapy provided herein and one or more additional agents (e.g., anti-cancer agents).

Acceptable carriers, excipients, or stabilizers are non-toxic to recipients at the dosages and concentrations employed, and include, for example, one or more of: buffers such as phosphate, citrate, and other organic acids; antioxidants, including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, or m-cresol; low molecular weight polypeptides (e.g., less than about 10 residues); proteins such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); surfactants such as non-ionic surfactants; or polymers such as polyethylene glycol (PEG).

The active ingredients may be entrapped in microcapsules. Such microcapsules may be prepared, for example, by coacervation techniques or by interfacial polymerization, for example, hydroxymethylcellulose or gelatin-microcapsules and poly-(methylmethacylate) microcapsules, respectively; in colloidal drug delivery systems (for example, liposomes, albumin microspheres, microemulsions, nano-particles and nano-capsules); or in macroemulsions. Such techniques are known in the art.

Sustained-release compositions may be prepared. Suitable examples of sustained-release compositions include semi-permeable matrices of solid hydrophobic polymers containing an anti-cancer therapy of the disclosure. Such matrices may be in the form of shaped articles, e.g., films, or microcapsules. Examples of sustained-release matrices include polyesters, hydrogels (for example, poly(2-hydroxyethyl-methacrylate), or poly(vinylalcohol)), polylactides, copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

A formulation provided herein may also contain more than one active compound, for example, those with complementary activities that do not adversely affect each other. The type and effective amounts of such medicaments depend, for example, on the amount and type of active compound(s) present in the formulation, and clinical parameters of the subjects.

For general information concerning formulations, see, e.g., Gilman et al. (eds.) The Pharmacological Bases of Therapeutics, 8th Ed., Pergamon Press, 1990; A. Gennaro (ed.), Remington's Pharmaceutical Sciences, 18th Edition, Mack Publishing Co., Pennsylvania, 1990; Avis et al. (eds.) Pharmaceutical Dosage Forms: Parenteral Medications Dekker, New York, 1993; Lieberman et al. (eds.) Pharmaceutical Dosage Forms: Tablets Dekker, New York, 1990; Lieberman et al. (eds.), Pharmaceutical Dosage Forms: Disperse Systems Dekker, New York, 1990; and Walters (ed.) Dermatological and Transdermal Formulations (Drugs and the Pharmaceutical Sciences), Vol 1 19, Marcel Dekker, 2002.

Formulations to be used for in vivo administration are sterile. This is readily accomplished by filtration through sterile filtration membranes or other methods known in the art.

In some embodiments, an anti-cancer therapy of the disclosure is administered as a monotherapy. In some embodiments, the anti-cancer therapy is administered in combination with one or more additional anti-cancer therapies or treatments, e.g., as described herein. In some embodiments, the one or more additional anti-cancer therapies or treatments include one or more anti-cancer therapies described herein. In some embodiments, the methods of the present disclosure comprise administration of any combination of any of the anti-cancer therapies provided herein. In some embodiments, the additional anti-cancer therapy comprises one or more of surgery, radiotherapy, chemotherapy, anti-angiogenic therapy, anti-DNA repair therapy, and anti-inflammatory therapy. In some embodiments, the additional anti-cancer therapy comprises an anti-neoplastic agent, a chemotherapeutic agent, a growth inhibitory agent, an anti-angiogenic agent, a radiation therapy, a cytotoxic agent, or combinations thereof. In some embodiments, an anti-cancer therapy may be administered in conjunction with a chemotherapy or chemotherapeutic agent. In some embodiments, the chemotherapy or chemotherapeutic agent is a platinum-based agent (including, without limitation cisplatin, carboplatin, oxaliplatin, and staraplatin). In some embodiments, an anti-cancer therapy may be administered in conjunction with a radiation therapy. In some embodiments, the anti-cancer therapy for use in any of the methods described herein (e.g., as monotherapy or in combination with another therapy or treatment) is an anti-cancer therapy or treatment described by Pietrantonio et al., J Natl Cancer Inst (2017) 109(12) and/or by Wang et al., Cancers (2020) 12(2):426, which are hereby incorporated by reference.

### D. Reporting

In some embodiments, the methods provided herein comprise generating a report, and/or providing a report to party.

In some embodiments, a report according to the present disclosure comprises information about one or more of: a fusion nucleic acid molecule or polypeptide of the disclosure, e.g., an ALK, BRAF, EGFR, ERBB2, FGFR1, FGFR2, FGFR3, MET, NTRK1, RAF1, RET, or ROS1 fusion nucleic acid molecule described herein (e.g., in Tables 1-6, and/or in the Examples herein), or a fusion polypeptide encoded by such a fusion nucleic acid molecule; a cancer of the disclosure, e.g., comprising a fusion nucleic acid molecule or polypeptide of the disclosure; or a treatment, a therapy, or one or more treatment options for an individual having a cancer, such as a cancer of the disclosure (e.g., comprising a fusion nucleic acid molecule or polypeptide described herein).

In some embodiments, a report according to the present disclosure comprises information about the presence or absence of a fusion nucleic acid molecule or polypeptide of the disclosure, e.g., an ALK, BRAF, EGFR, ERBB2, FGFR1, FGFR2, FGFR3, MET, NTRK1, RAF1, RET, or ROS1 fusion nucleic acid molecule described herein (e.g., in Tables 1-6, and/or in the Examples herein), or a fusion polypeptide encoded by such a fusion nucleic acid molecule, in a sample obtained from an individual, such as an individual having a cancer, e.g., a cancer provided herein. In one embodiment, a report according to the present disclosure indicates that a fusion nucleic acid molecule or polypeptide of the disclosure is present in a sample obtained from the individual. In one embodiment, a report according to the present disclosure indicates that a fusion nucleic acid molecule or polypeptide of the disclosure is not present in a sample obtained from the individual. In one embodiment, a report according to the present disclosure indicates that a fusion nucleic acid molecule or polypeptide of the disclosure has been detected in a sample obtained from the individual. In one embodiment, a report according to the present disclosure indicates that a fusion nucleic acid molecule or polypeptide of the disclosure has not been detected in a sample obtained from the individual. In some embodiments, the report comprises an identifier for the individual from which the sample was obtained.

In some embodiments, the report includes information on the role of a fusion nucleic acid molecule or polypeptide of the disclosure, e.g., an ALK, BRAF, EGFR, ERBB2, FGFR1, FGFR2, FGFR3, MET, NTRK1, RAF1, RET, or ROS1 fusion nucleic acid molecule described herein (e.g., in Tables 1-6, and/or in the Examples herein), or a fusion polypeptide encoded by such a fusion nucleic acid molecule, or its wild type counterparts, in disease, such as in cancer. Such information can include one or more of: information on prognosis of a cancer, such as a cancer provided herein, e.g., comprising a fusion nucleic acid molecule or polypeptide described herein; information on resistance of a cancer, such as a cancer provided herein, e.g., comprising a fusion nucleic acid molecule or polypeptide described herein, to one or more treatments; information on potential or suggested therapeutic options (e.g., such as an anti-cancer therapy provided herein, or a treatment selected or identified according to the methods provided herein); or information on therapeutic options that should be avoided. In some embodiments, the report includes information on the likely effectiveness, acceptability, and/or advisability of applying a therapeutic option (e.g., such as an anti-cancer therapy provided herein, or a treatment selected or identified according to the methods provided herein) to an individual having a cancer, such as a cancer provided herein, e.g., comprising a fusion nucleic acid molecule or polypeptide described herein and identified in the report. In some embodiments, the report includes information or a recommendation on the administration of a treatment (e.g., an anti-cancer therapy provided herein, or a treatment selected or identified according to the methods provided herein). In some embodiments, the information or recommendation includes the dosage of the treatment and/or a treatment regimen (e.g., in combination with other treatments, such as a second therapeutic agent). In some embodiments, the report comprises information or a recommendation for at least one, at least two, at least three, at least four, at least five, at least six, at least seven, at least eight, at least nine, at least ten, or more treatments.

Also provided herein are methods of generating a report according to the present disclosure. In some embodiments, a report according to the present disclosure is generated by a method comprising one or more of the following steps: obtaining a sample, such as a sample described herein, from an individual, e.g., an individual having a cancer, such as a cancer provided herein; detecting a fusion nucleic acid molecule or polypeptide of the disclosure, e.g., an ALK, BRAF, EGFR, ERBB2, FGFR1, FGFR2, FGFR3, MET, NTRK1, RAF1, RET, or ROS1 fusion nucleic acid molecule described herein (e.g., in Tables 1-6, and/or in the Examples herein), or a fusion polypeptide encoded by such a fusion nucleic acid molecule, in the sample, or acquiring knowledge of the presence of the fusion nucleic acid molecule or polypeptide of the disclosure in the sample; and generating a report. In some embodiments, a report generated according to the methods provided herein comprises one or more of: information about the presence or absence of a fusion nucleic acid molecule or polypeptide of the disclosure, e.g., an ALK, BRAF, EGFR, ERBB2, FGFR1, FGFR2, FGFR3, MET, NTRK1, RAF1, RET, or ROS1 fusion nucleic acid molecule described herein (e.g., in Tables 1-6, and/or in the Examples herein), or a fusion polypeptide encoded by such a fusion nucleic acid molecule, in the sample; an identifier for the individual from which the sample was obtained; information on the role of the fusion nucleic acid molecule or polypeptide of the disclosure, or its wild type counterparts, in disease (e.g., such as in cancer); information on prognosis, resistance, or potential or suggested therapeutic options (such as an anti-cancer therapy provided herein, or a treatment selected or identified according to the methods provided herein); information on the likely effectiveness, acceptability, or the advisability of applying a therapeutic option (such as an anti-cancer therapy provided herein, or a treatment selected or identified according to the methods provided herein) to the individual; a recommendation or information on the administration of a treatment (such as an anti-cancer therapy provided herein, or a treatment selected or identified according to the methods provided herein); or a recommendation or information on the dosage or treatment regimen of a treatment (such as an anti-cancer therapy provided herein, or a treatment selected or identified according to the methods provided herein), e.g., in combination with other treatments (e.g., a second therapeutic agent). In some embodiments, the report generated is a personalized cancer report.

A report according to the present disclosure may be in an electronic, web-based, or paper form. The report may be provided to an individual or a patient (e.g., an individual or a patient with a cancer, such as a cancer provided herein, e.g., comprising a fusion nucleic acid molecule or polypeptide of the disclosure), or to an individual or entity other than the individual or patient (e.g., other than the individual or patient with the cancer), such as one or more of a caregiver, a physician, an oncologist, a hospital, a clinic, a third party payor, an insurance company, or a government entity. In some embodiments, the report is provided or delivered to the individual or entity within any of about 1 day or more, about 7 days or more, about 14 days or more, about 21 days or more, about 30 days or more, about 45 days or more, or about 60 days or more from obtaining a sample from an individual (e.g., an individual having a cancer). In some embodiments, the report is provided or delivered to an individual or entity within any of about 1 day or more, about 7 days or more, about 14 days or more, about 21 days or more, about 30 days or more, about 45 days or more, or about 60 days or more from detecting a fusion nucleic acid molecule or polypeptide of the disclosure in a sample obtained from an individual (e.g., an individual having a cancer). In some embodiments, the report is provided or delivered to an individual or entity within any of about 1 day or more, about 7 days or more, about 14 days or more, about 21 days or more, about 30 days or more, about 45 days or more, or about 60 days or more from acquiring knowledge of the presence of the fusion nucleic acid molecule or polypeptide of the disclosure in a sample obtained from an individual (e.g., an individual having a cancer).

### E. Software, Systems, and Devices

In some other aspects, provided herein are non-transitory computer-readable storage media. In some embodiments, the non-transitory computer-readable storage media comprise one or more programs for execution by one or more processors of a device, the one or more programs including instructions which, when executed by the one or more processors, cause the device to perform the method according to any of the embodiments described herein.

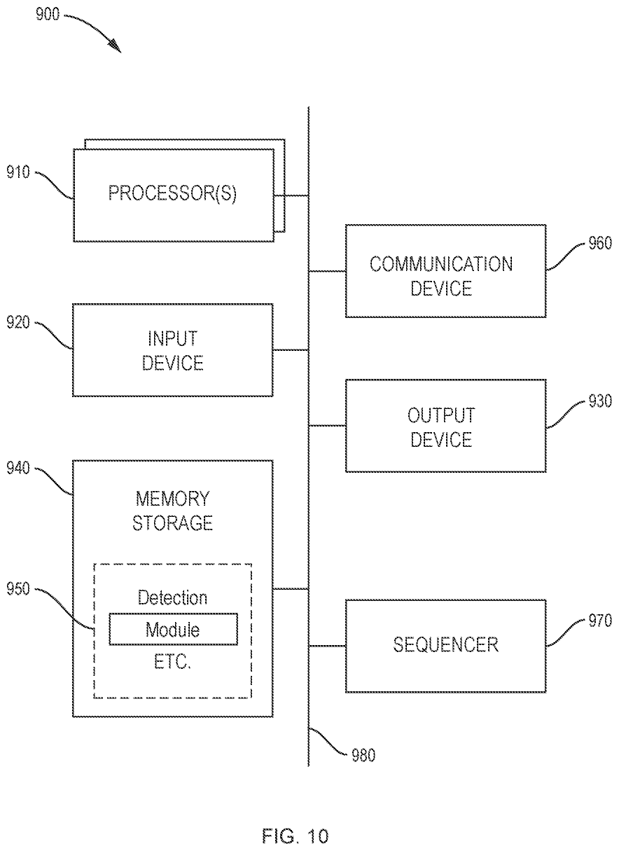

FIG. 10 illustrates an example of a computing device or system in accordance with one embodiment. Device 900 can be a host computer connected to a network. Device 900 can be a client computer or a server. As shown in FIG. 10, device 900 can be any suitable type of microprocessor-based device, such as a personal computer, workstation, server or handheld computing device (portable electronic device) such as a phone or tablet. The device can include, for example, one or more processor(s) 910, input devices 920, output devices 930, memory or storage devices 940, communication devices 960, and nucleic acid sequencers 970. Software 950 residing in memory or storage device 940 may comprise, e.g., an operating system as well as software for executing the methods described herein, e.g., for detecting a fusion nucleic acid molecule of the disclosure. Input device 920 and output device 930 can generally correspond to those described herein, and can either be connectable or integrated with the computer.

Input device 920 can be any suitable device that provides input, such as a touch screen, keyboard or keypad, mouse, or voice-recognition device. Output device 930 can be any suitable device that provides output, such as a touch screen, haptics device, or speaker.

Storage 940 can be any suitable device that provides storage (e.g., an electrical, magnetic or optical memory including a RAM (volatile and non-volatile), cache, hard drive, or removable storage disk). Communication device 960 can include any suitable device capable of transmitting and receiving signals over a network, such as a network interface chip or device. The components of the computer can be connected in any suitable manner, such as via a wired media (e.g., a physical system bus 980, Ethernet connection, or any other wire transfer technology) or wirelessly (e.g., Bluetooth®, Wi-Fi®, or any other wireless technology).

Software module 950, which can be stored as executable instructions in storage 940 and executed by processor(s) 910, can include, for example, an operating system and/or the processes that embody the functionality of the methods of the present disclosure, e.g., for detecting a fusion nucleic acid molecule of the disclosure (e.g., as embodied in the devices as described herein).

Software module 950 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 940, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 950 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

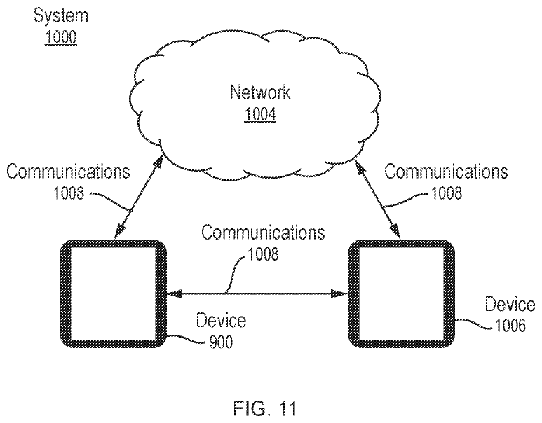

Device 900 may be connected to a network (e.g., network 1004, as shown in FIG. 11 and described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 900 can be implemented using any operating system, e.g., an operating system suitable for operating on the network. Software module 950 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 910.

Device 900 can further include a sequencer 970, which can be any suitable nucleic acid sequencing instrument. Exemplary sequencers can include, without limitation, Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio RS system.

FIG. 11 illustrates an example of a computing system in accordance with one embodiment. In computing system 1000, device 900 (e.g., as described above and illustrated in FIG. 10) is connected to network 1004, which is also connected to device 1006. In some embodiments, device 1006 is a sequencer. Exemplary sequencers can include, without limitation, Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio RS system.

Devices 900 and 1006 may communicate, e.g., using suitable communication interfaces via network 1004, such as a Local Area Network (LAN), Virtual Private Network (VPN), or the Internet. In some embodiments, network 1004 can be, for example, the Internet, an intranet, a virtual private network, a cloud network, a wired network, or a wireless network. Devices 900 and 1006 may communicate, in part or in whole, via wireless or hardwired communications, such as Ethernet, IEEE 802.11b wireless, or the like. Additionally, devices 900 and 1006 may communicate, e.g., using suitable communication interfaces, via a second network, such as a mobile/cellular network. Communication between devices 900 and 1006 may further include or communicate with various servers such as a mail server, mobile server, media server, telephone server, and the like. In some embodiments, devices 900 and 1006 can communicate directly (instead of, or in addition to, communicating via network 1004), e.g., via wireless or hardwired communications, such as Ethernet, IEEE 802.11b wireless, or the like. In some embodiments, devices 900 and 1006 communicate via communications 1008, which can be a direct connection or can occur via a network (e.g., network 1004).

One or all of devices 900 and 1006 generally include logic (e.g., http web server logic) or are programmed to format data, accessed from local or remote databases or other sources of data and content, for providing and/or receiving information via network 1004 according to various examples described herein.

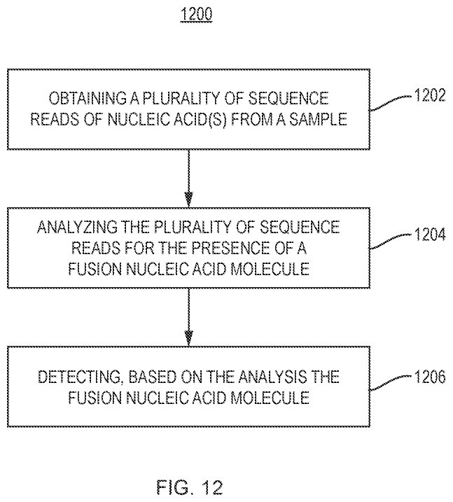

FIG. 12 illustrates an exemplary process 1200 for detecting a fusion nucleic acid molecule of the disclosure in a sample, in accordance with some embodiments of the present disclosure. Process 1200 is performed, for example, using one or more electronic devices implementing a software program. In some examples, process 1200 is performed using a client-server system, and the blocks of process 1200 are divided up in any manner between the server and a client device. In other examples, the blocks of process 1200 are divided up between the server and multiple client devices. Thus, while portions of process 1200 are described herein as being performed by particular devices of a client-server system, it will be appreciated that process 1200 is not so limited. In some embodiments, the executed steps can be executed across many systems, e.g., in a cloud environment. In other examples, process 1200 is performed using only a client device or only multiple client devices. In process 1200, some blocks are, optionally, combined, the order of some blocks is, optionally, changed, and some blocks are, optionally, omitted. In some examples, additional steps may be performed in combination with the process 1200. Accordingly, the operations as illustrated (and described in greater detail below) are exemplary by nature and, as such, should not be viewed as limiting.

At block 1202, a plurality of sequence reads of one or more nucleic acid molecules is obtained, wherein the one or more nucleic acid molecules are derived from a sample obtained from an individual, e.g., as described herein. In some embodiments, the sample is obtained from an individual having a cancer, such as a cancer described herein. In some embodiments, the sequence reads are obtained using a sequencer, e.g., as described herein or otherwise known in the art. In some embodiments, the nucleic acid molecules comprise one or more nucleic acid molecules corresponding to: a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein (e.g., in Tables 1-6, and/or in the Examples herein); or a gene involved in a fusion nucleic acid molecule of the disclosure; or fragments thereof. Optionally, prior to obtaining the sequence reads, the sample is purified, enriched (e.g., for nucleic acid(s) corresponding to: a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein (e.g., in Tables 1-6, and/or in the Examples herein); or a gene involved in a fusion nucleic acid molecule of the disclosure; or fragments thereof), and/or subjected to PCR amplification. At block 1204, an exemplary system (e.g., one or more electronic devices) analyzes the plurality of sequence reads for the presence of a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein (e.g., in Tables 1-6, and/or in the Examples herein), or a fragment thereof. At block 1206, the system detects (e.g., based on the analysis) a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein (e.g., in Tables 1-6, and/or in the Examples herein), or a fragment thereof, in the sample.

In some embodiments, the fusion nucleic acid molecule is an ALK, BRAF, EGFR, ERBB2, FGFR1, FGFR2, FGFR3, MET, RAF1, RET, or ROS1 fusion nucleic acid molecule listed in Table 1. In some embodiments, the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, comprising or resulting from a Breakpoint 1 and/or a Breakpoint 2 corresponding to the ALK, BRAF, EGFR, ERBB2, FGFR1, FGFR2, FGFR3, MET, RAF1, RET, or ROS1 fusion nucleic acid molecule as listed in Table 3. In some embodiments, the cancer is any of the cancers described herein, e.g., above. In some embodiments, the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, and the cancer is the cancer corresponding to the ALK, BRAF, EGFR, ERBB2, FGFR1, FGFR2, FGFR3, MET, RAF1, RET, or ROS1 fusion nucleic acid molecule as listed in Table 4. In some embodiments, the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, the cancer is the cancer corresponding to the ALK, BRAF, EGFR, ERBB2, FGFR1, FGFR2, FGFR3, MET, RAF1, RET, or ROS1 fusion nucleic acid molecule as listed in Table 5, and the fusion nucleic acid molecule comprises or results from a Breakpoint 1 and/or a Breakpoint 2 corresponding to the ALK, BRAF, EGFR, ERBB2, FGFR1, FGFR2, FGFR3, MET, RAF1, RET, or ROS1 fusion nucleic acid molecule as listed in Table 5.

In some embodiments, the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2. In some embodiments, the fusion nucleic acid molecule comprises or results from a Breakpoint 1 and/or a Breakpoint 2 corresponding to the ALK, BRAF, ERBB2, FGFR1, FGFR2, FGFR3, MET, NTRK1, RAF1, RET, or ROS1 fusion nucleic acid molecule as listed in Table 6.

In some embodiments of any of the methods, systems, devices, non-transitory computer readable storage media, or processes of the disclosure, the plurality of sequence reads is obtained by sequencing nucleic acids obtained from any of the samples described herein, e.g., tissue and/or liquid biopsies, etc. In some embodiments, the sample is obtained from the cancer. In some embodiments, the sample comprises a tissue biopsy sample, a liquid biopsy sample, or a normal control. In some embodiments, the sample is from a tumor biopsy, tumor specimen, or circulating tumor cell. In some embodiments, the sample is a liquid biopsy sample and comprises blood, plasma, cerebrospinal fluid, sputum, stool, urine, or saliva. In some embodiments, the sample comprises cells and/or nucleic acids from the cancer. In some embodiments, the sample comprises mRNA, DNA, circulating tumor DNA (ctDNA), cell-free DNA, or cell-free RNA from the cancer. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof.

In some embodiments of any of the methods, systems, devices, non-transitory computer readable storage media, or processes of the disclosure, the plurality of sequence reads is obtained by sequencing. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the massively parallel sequencing technique comprises next generation sequencing (NGS).

In some embodiments of any of the methods, systems, devices, non-transitory computer readable storage media, or processes of the disclosure, the disclosed methods for determining the presence or absence of a fusion nucleic acid molecule of the disclosure may be implemented as part of a genomic profiling process that comprises identification of the presence of variant sequences at one or more gene loci in a sample derived from an individual as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), a next-generation sequencing (NGS) approach used to assess hundreds of genes (including relevant cancer biomarkers) in a single assay. Inclusion of the disclosed methods for determining the presence or absence of a fusion nucleic acid molecule of the disclosure as part of a genomic profiling process can improve the validity of, e.g., disease detection calls, made on the basis of the genomic profile by, for example, independently confirming the presence of the fusion nucleic acid molecule of the disclosure in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the individual may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

Accordingly, in some embodiments of any of the methods, systems, devices, non-transitory computer readable storage media, or processes of the disclosure, a genomic profile for the sample or for the individual is generated based at least in part on detecting a fusion nucleic acid molecule of the disclosure, or a fragment thereof, in the sample. In some embodiments, the individual is administered a treatment based at least in part on the genomic profile, e.g., a treatment described herein. In some embodiments, the genomic profile further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the genomic profile further comprises results from a nucleic acid sequencing-based test. In some embodiments, the genomic profile further comprises/indicates/comprises information on presence or absence of mutations in one or more additional genes, e.g., a panel of known oncogenes and/or tumor suppressors. In some embodiments, the genomic profile is obtained from a genomic profiling assay (such as a cancer- or tumor-related genomic profiling assay), e.g., as obtained using any of the sequencing methodologies described herein. In some embodiments, the genomic profile includes information from whole-genome or whole-exome sequencing. In some embodiments, the genomic profile includes information from targeted sequencing. In some embodiments, the genomic profile includes information from NGS. In some embodiments, the genomic profile comprises/indicates/comprises information on presence or absence of mutations such as short variant alterations (e.g., a base substitution, insertion, or deletion), copy-number alterations (e.g., an amplification or a homozygous deletion), and/or rearrangements (e.g., a gene fusion or other genomic or chromosomal rearrangement) of one or more genes, e.g., a panel of known oncogenes and/or tumor suppressors.

The method steps of the methods described herein are intended to include any suitable method of causing one or more other parties or entities to perform the steps, unless a different meaning is expressly provided or otherwise clear from the context. Such parties or entities need not be under the direction or control of any other party or entity, and need not be located within a particular jurisdiction. Thus, for example, a description or recitation of “adding a first number to a second number” includes causing one or more parties or entities to add the two numbers together. For example, if person X engages in an arm's length transaction with person Y to add the two numbers, and person Y indeed adds the two numbers, then both persons X and Y perform the step as recited: person Y by virtue of the fact that he actually added the numbers, and person X by virtue of the fact that he caused person Y to add the numbers. Furthermore, if person X is located within the United States and person Y is located outside the United States, then the method is performed in the United States by virtue of person X's participation in causing the step to be performed.

### IV. Articles of Manufacture or Kits

Provided herein are kits or articles of manufacture comprising one or more reagents for detecting a fusion nucleic acid molecule of the disclosure in a sample, e.g., an ALK, BRAF, EGFR, ERBB2, FGFR1, FGFR2, FGFR3, MET, NTRK1, RAF1, RET, or ROS1 fusion nucleic acid molecule described herein and/or in Tables 1-6, and/or in the Examples herein; or a fusion polypeptide encoded by a fusion nucleic acid molecule of the disclosure.

In some embodiments, the kits or articles of manufacture comprise one or more probes of the disclosure for detecting a fusion nucleic acid molecule of the disclosure in a sample, e.g., an ALK, BRAF, EGFR, ERBB2, FGFR1, FGFR2, FGFR3, MET, NTRK1, RAF1, RET, or ROS1 fusion nucleic acid molecule described herein and/or in Tables 1-6, and/or in the Examples herein, e.g., according to any detection method known in the art or described herein. In some embodiments, the kits or articles of manufacture comprise one or more baits (e.g., one or more bait molecules) of the disclosure for detecting a fusion nucleic acid molecule of the disclosure in a sample, e.g., an ALK, BRAF, EGFR, ERBB2, FGFR1, FGFR2, FGFR3, MET, NTRK1, RAF1, RET, or ROS1 fusion nucleic acid molecule described herein and/or in Tables 1-6, and/or in the Examples herein, e.g., according to any detection method known in the art or described herein. In some embodiments, the kits or articles of manufacture comprise one or more oligonucleotides (e.g., one or more primers) of the disclosure for detecting a fusion nucleic acid molecule of the disclosure in a sample, e.g., an ALK, BRAF, EGFR, ERBB2, FGFR1, FGFR2, FGFR3, MET, NTRK1, RAF1, RET, or ROS1 fusion nucleic acid molecule described herein and/or in Tables 1-6, and/or in the Examples herein, e.g., according to any detection method known in the art or described herein. In some embodiments of any of the kits or articles of manufacture provided herein, the kit or article of manufacture comprises a reagent (e.g., one or more oligonucleotides, primers, probes or baits of the present disclosure) for detecting a wild-type counterpart of a fusion nucleic acid molecule of the disclosure (e.g., a wild type ALK, BRAF, EGFR, ERBB2, FGFR1, FGFR2, FGFR3, MET, NTRK1, RAF1, RET, or ROS1 gene, and/or a wild type fusion partner gene described herein and/or in Tables 1-6, and/or in the Examples herein). In some embodiments, one or more oligonucleotides, primers, probes or baits are capable of hybridizing to a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein and/or in Tables 1-6, and/or in the Examples herein, or to a wild-type counterpart of the fusion nucleic acid molecule (e.g., a wild type ALK, BRAF, EGFR, ERBB2, FGFR1, FGFR2, FGFR3, MET, NTRK1, RAF1, RET, or ROS1 gene, and/or a wild type fusion partner gene described herein and/or in Tables 1-6, and/or in the Examples herein). In some embodiments, the one or more oligonucleotides, primers, probes or baits of the present disclosure are capable of distinguishing a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein and/or in Tables 1-6, and/or in the Examples herein, from a wild-type counterpart of the fusion nucleic acid molecule (e.g., a wild type ALK, BRAF, EGFR, ERBB2, FGFR1, FGFR2, FGFR3, MET, NTRK1, RAF1, RET, or ROS1 gene, and/or a wild type fusion partner gene described herein and/or in Tables 1-6, and/or in the Examples herein). In some embodiments, the kit is for use according to any method of detecting fusion nucleic acid molecules known in the art or described herein, such as sequencing, PCR, in situ hybridization methods, a nucleic acid hybridization assay, an amplification-based assay, a PCR-RFLP assay, real-time PCR, sequencing, next-generation sequencing, a screening analysis, FISH, spectral karyotyping, MFISH, comparative genomic hybridization, in situ hybridization, sequence-specific priming (SSP) PCR, HPLC, and mass-spectrometric genotyping. In some embodiments, a kit provided herein further comprises instructions for detecting a fusion nucleic acid molecule of the disclosure, e.g., using one or more oligonucleotides, primers, probes or baits of the present disclosure.

In some embodiments, the kits or articles of manufacture comprise one or more antibodies or antibody fragments of the disclosure for detecting a fusion polypeptide encoded by a fusion nucleic acid molecule of the disclosure in a sample, e.g., an ALK, BRAF, EGFR, ERBB2, FGFR1, FGFR2, FGFR3, MET, NTRK1, RAF1, RET, or ROS1 fusion nucleic acid molecule described herein and/or in Tables 1-6, and/or in the Examples herein, e.g., according to any detection method known in the art or described herein. In some embodiments, the kit or article of manufacture comprises a reagent (e.g., one or more antibodies of the present disclosure) for detecting the wild-type counterparts of a fusion polypeptide provided herein (e.g., a wild type ALK, BRAF, EGFR, ERBB2, FGFR1, FGFR2, FGFR3, MET, NTRK1, RAF1, RET, or ROS1 polypeptide, and/or a wild type polypeptide encoded by a fusion partner gene described herein and/or in Tables 1-6, and/or in the Examples herein). In some embodiments, the kits or articles of manufacture comprise one or more antibodies of the present disclosure capable of binding to a fusion polypeptide provided herein, or to wild-type counterparts of the fusion polypeptide provided herein (e.g., a wild type ALK, BRAF, EGFR, ERBB2, FGFR1, FGFR2, FGFR3, MET, NTRK1, RAF1, RET, or ROS1 polypeptide, and/or a wild type polypeptide encoded by a fusion partner gene described herein and/or in Tables 1-6, and/or in the Examples herein). In some embodiments, the kit is for use according to any protein or polypeptide detection assay known in the art or described herein, such as mass spectrometry (e.g., tandem mass spectrometry), a reporter assay (e.g., a fluorescence-based assay), immunoblots such as a Western blot, immunoassays such as enzyme-linked immunosorbent assays (ELISA), immunohistochemistry, other immunological assays (e.g., fluid or gel precipitin reactions, immunodiffusion, immunoelectrophoresis, radioimmunoassay (RIA), immunofluorescent assays), and analytic biochemical methods (e.g., electrophoresis, capillary electrophoresis, high performance liquid chromatography (HPLC), thin layer chromatography (TLC), hyperdiffusion chromatography). In some embodiments, the kit further comprises instructions for detecting a fusion polypeptide of the disclosure, e.g., using one or more antibodies of the present disclosure.

Further provided herein are kits or articles of manufacture comprising an anti-cancer therapy, such as an anti-cancer therapy described herein, and a package insert comprising instructions for using the anti-cancer therapy in a method of treating or delaying progression of cancer, e.g., by administration to an individual from whom a sample comprising a fusion nucleic acid molecule or polypeptide of the disclosure has been obtained. In some embodiments, the anti-cancer therapy is any of the anti-cancer therapies described herein for use in any of the methods for treating or delaying progression of cancer of the disclosure.

The kit or article of manufacture may include, for example, a container and a label or package insert on or associated with the container. Suitable containers include, for example, bottles, vials, syringes, and the like. The container may be formed from a variety of materials such as glass or plastic. The container holds or contains a composition comprising one or more reagents for detecting a fusion nucleic acid molecule or polypeptide of the disclosure (e.g., one or more oligonucleotides, primers, probes, baits, antibodies or antibody fragments of the present disclosure) or one or more anti-cancer therapies of the disclosure. In some embodiments, the container holds or contains a composition comprising one or more anti-cancer therapies of the disclosure and may have a sterile access port (e.g., the container may be an intravenous solution bag or a vial having a stopper pierceable by a hypodermic injection needle).

The kit or article of manufacture may further include a second container comprising a diluent or buffer, e.g., a pharmaceutically-acceptable diluent buffer, such as bacteriostatic water for injection (BWFI), phosphate-buffered saline, Ringer's solution, and/or dextrose solution. The article of manufacture may further include other materials desirable from a commercial and user standpoint, including other buffers, diluents, filters, needles, and syringes.

The kit or article of manufacture of the present disclosure also includes information or instructions, for example in the form of a package insert, indicating that the one or more reagents and/or anti-cancer therapies are used for detecting a fusion nucleic acid molecule or polypeptide of the disclosure, or for treating cancer, as described herein. The insert or label may take any form, such as paper or on electronic media such as a magnetically recorded medium (e.g., floppy disk), a CD-ROM, a Universal Serial Bus (USB) flash drive, and the like. The label or insert may also include other information concerning the pharmaceutical compositions and dosage forms in the kit or article of manufacture.

### V. Expression Vectors, Host Cells and Recombinant Cells

Provided herein are vectors comprising or encoding a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein and/or in Tables 1-6, and/or in the Examples herein, or a bait, a probe, or an oligonucleotide described herein, or fragments thereof.

In some embodiments, a vector provided herein comprises or encodes a fusion nucleic acid molecule of the disclosure, e.g., an ALK, BRAF, EGFR, ERBB2, FGFR1, FGFR2, FGFR3, MET, NTRK1, RAF1, RET, or ROS1 fusion nucleic acid molecule described herein and/or in Tables 1-6, and/or in the Examples herein, or a nucleic acid molecule encoding a fusion polypeptide described herein.

In some embodiments, a vector provided herein is a nucleic acid molecule capable of transporting another nucleic acid to which it has been linked (e.g., fusion nucleic acid molecules, baits, probes, or oligonucleotides described herein, or fragments thereof). In some embodiments, a vector is a plasmid, a cosmid or a viral vector. The vector may be capable of autonomous replication, or it can integrate into a host DNA. Viral vectors (e.g., comprising fusion nucleic acid molecules, baits, probes, or oligonucleotides described herein, or fragments thereof) are also contemplated herein, including, e.g., replication defective retroviruses, adenoviruses and adeno-associated viruses.

In some embodiments, a vector provided herein comprises a fusion nucleic acid molecule, a bait, a probe, or an oligonucleotide of the disclosure in a form suitable for expression thereof in a host cell. In some embodiments, the vector includes one or more regulatory sequences operatively linked to the nucleotide sequence to be expressed. In some embodiments, the one or more regulatory sequences include promoters (e.g., promoters derived from polyoma, Adenovirus 2, cytomegalovirus and Simian Virus 40), enhancers, and other expression control elements (e.g., polyadenylation signals). In some embodiments, a regulatory sequence directs constitutive expression of a nucleotide sequence (e.g., fusion nucleic acid molecules, baits, probes, or oligonucleotides described herein, or fragments thereof). In some embodiments, a regulatory sequence directs tissue-specific expression of a nucleotide sequence (e.g., fusion nucleic acid molecules, baits, probes, or oligonucleotides described herein, or fragments thereof). In some embodiments, a regulatory sequence directs inducible expression of a nucleotide sequence (e.g., fusion nucleic acid molecules, baits, probes, or oligonucleotides described herein, or fragments thereof). Examples of inducible regulatory sequences include, without limitation, promoters regulated by a steroid hormone, by a polypeptide hormone, or by a heterologous polypeptide, such as a tetracycline-inducible promoter. Examples of tissue- or cell-type-specific regulatory sequences include, without limitation, the albumin promoter, lymphoid-specific promoters, promoters of T cell receptors or immunoglobulins, neuron-specific promoters, pancreas-specific promoters, mammary gland-specific promoters, and developmentally-regulated promoters. In some embodiments, a vector provided herein comprises or encodes a fusion nucleic acid molecule, a bait, a probe, or an oligonucleotide of the disclosure in the sense or the anti-sense orientation. In some embodiments, a vector (e.g., an expression vector) provided herein is introduced into host cells to thereby produce a polypeptide, e.g., a fusion polypeptide described herein, or a fragment or mutant form thereof.

In some embodiments, the design of a vector provided herein depends on such factors as the choice of the host cell to be transformed, the level of expression desired, and the like. In some embodiments, expression vectors are designed for the expression of the fusion nucleic acid molecules, baits, probes, or oligonucleotides described herein, or fragments thereof, in prokaryotic or eukaryotic cells, such as E. coli cells, insect cells (e.g., using baculovirus expression vectors), yeast cells, or mammalian cells. In some embodiments, a vector described herein is transcribed and translated in vitro, for example using T7 promoter regulatory sequences and T7 polymerase. In some embodiments, a vector (e.g., an expression vector) provided herein comprises or encodes a fusion nucleic acid molecule described herein, wherein the nucleotide sequence of the fusion nucleic acid molecule described herein has been altered (e.g., codon optimized) so that the individual codons for each encoded amino acid are those preferentially utilized in the host cell.

Also provided herein are host cells, e.g., comprising fusion nucleic acid molecules, fusion polypeptides, baits, probes, vectors, or oligonucleotides of the disclosure. In some embodiments, a host cell (e.g., a recombinant host cell or recombinant cell) comprises a vector described herein (e.g., an expression vector described herein). In some embodiments, a fusion nucleic acid molecule, bait, probe, vector, or oligonucleotide provided herein further includes sequences which allow it to integrate into the host cell's genome (e.g., through homologous recombination at a specific site). In some embodiments, a host cell provided herein is a prokaryotic or eukaryotic cell. Non limiting examples of host cells include, without limitation, bacterial cells (e.g., E. coli), insect cells, yeast cells, or mammalian cells (e.g., human cells, rodent cells, mouse cells, rabbit cells, pig cells, Chinese hamster ovary cells (CHO), or COS cells, e.g., COS-7 cells, CV-1 origin SV40 cells). A host cell described herein includes the particular host cell, as well as the progeny of such a cell. Because certain modifications may occur in succeeding generations due to either mutation or environmental influences, such progeny may not, in fact, be identical to the parent host cell.

Fusion nucleic acid molecules, baits, probes, vectors, or oligonucleotides of the disclosure may be introduced into host cells using any suitable method known in the art, such as conventional transformation or transfection techniques (e.g., using calcium phosphate or calcium chloride co-precipitation, DEAE-dextran-mediated transfection, lipofection, or electroporation).

Also provided herein are methods of producing a fusion polypeptide of the disclosure, e.g., by culturing a host cell described herein (e.g., into which a recombinant expression vector encoding a polypeptide has been introduced) in a suitable medium such that the fusion polypeptide is produced. In another embodiment, the method further includes isolating a fusion polypeptide from the medium or the host cell.

### VI. Exemplary Clauses

The following exemplary clauses are representative of some aspects of the invention: Exemplary Clause 1: A method of identifying an individual having a cancer who may benefit from a treatment comprising an anti-cancer therapy, the method comprising detecting in a sample from the individual a fusion nucleic acid molecule, or a fusion polypeptide encoded by the fusion nucleic acid molecule, wherein:
(a) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or
(b) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2; wherein detection of the fusion nucleic acid molecule or fusion polypeptide encoded by the fusion nucleic acid molecule in the sample identifies the individual as one who may benefit from the treatment comprising the anti-cancer therapy.

Exemplary Clause 2: A method of selecting a treatment for an individual having a cancer, the method comprising detecting in a sample from the individual a fusion nucleic acid molecule, or a fusion polypeptide encoded by the fusion nucleic acid molecule, wherein:
(a) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or
(b) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2, wherein detection of the fusion nucleic acid molecule or fusion polypeptide encoded by the fusion nucleic acid molecule in the sample identifies the individual as one who may benefit from a treatment comprising an anti-cancer therapy.

Exemplary Clause 3: A method of identifying one or more treatment options for an individual having a cancer, the method comprising:
(a) detecting in a sample from the individual a fusion nucleic acid molecule, or a fusion polypeptide encoded by the fusion nucleic acid molecule, wherein:
(i) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or
(ii) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2; and
(b) generating a report comprising one or more treatment options identified for the individual based at least in part on detection of the fusion nucleic acid molecule or fusion polypeptide encoded by the fusion nucleic acid molecule in the sample, wherein the one or more treatment options comprise an anti-cancer therapy.

Exemplary Clause 4: A method of identifying one or more treatment options for an individual having a cancer, the method comprising:
(a) acquiring knowledge of a fusion nucleic acid molecule, or a fusion polypeptide encoded by the fusion nucleic acid molecule, in a sample from the individual, wherein:
(i) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or
(ii) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2; and
(b) generating a report comprising one or more treatment options identified for the individual based at least in part on said knowledge, wherein the one or more treatment options comprise an anti-cancer therapy.

Exemplary Clause 5: A method of selecting a treatment for an individual having cancer, comprising acquiring knowledge of a fusion nucleic acid molecule, or a fusion polypeptide encoded by the fusion nucleic acid molecule, in a sample from the individual, wherein:
(a) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or
(b) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2;
wherein responsive to the acquisition of said knowledge: (i) the individual is classified as a candidate to receive a treatment comprising an anti-cancer therapy; and/or (ii) the individual is identified as likely to respond to a treatment that comprises an anti-cancer therapy.

Exemplary Clause 6: A method of predicting survival of an individual having a cancer, comprising acquiring knowledge of a fusion nucleic acid molecule, or a fusion polypeptide encoded by the fusion nucleic acid molecule, in a sample from the individual, wherein:
(a) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or
(b) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2,
wherein responsive to the acquisition of said knowledge, the individual is predicted to have longer survival when treated with a treatment comprising an anti-cancer therapy, as compared to survival of an individual whose cancer does not comprise the fusion nucleic acid molecule or fusion polypeptide encoded by the fusion nucleic acid molecule.

Exemplary Clause 7: A method of predicting survival of an individual having a cancer treated with a treatment comprising an anti-cancer therapy, the method comprising acquiring knowledge of a fusion nucleic acid molecule, or a fusion polypeptide encoded by the fusion nucleic acid molecule, in a sample from the individual, wherein:
(a) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or
(b) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2,
wherein responsive to the acquisition of said knowledge, the individual is predicted to have longer survival when treated with a treatment comprising an anti-cancer therapy, as compared to an individual whose cancer does not exhibit the fusion nucleic acid molecule or fusion polypeptide encoded by the fusion nucleic acid molecule.

Exemplary Clause 8: A method of treating or delaying progression of cancer, comprising:
(a) acquiring knowledge of a fusion nucleic acid molecule, or a fusion polypeptide encoded by the fusion nucleic acid molecule, in a sample from an individual having a cancer, wherein:
(i) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or
(ii) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2; and
(b) responsive to said knowledge, administering to the individual an effective amount of a treatment that comprises an anti-cancer therapy.

Exemplary Clause 9: A method of treating or delaying progression of cancer, comprising administering to an individual having cancer an effective amount of a treatment that comprises an anti-cancer therapy, wherein the anti-cancer therapy is administered responsive to acquiring knowledge of a fusion nucleic acid molecule, or a fusion polypeptide encoded by the fusion nucleic acid molecule, in a sample from the individual, wherein:
(a) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or
(b) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2.

Exemplary Clause 10: A method of monitoring, evaluating or screening an individual having a cancer, comprising acquiring knowledge of a fusion nucleic acid molecule, or a fusion polypeptide encoded by the fusion nucleic acid molecule, in a sample from the individual, wherein:
(a) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or
(b) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2;
wherein responsive to the acquisition of said knowledge, the individual is predicted to have acquired resistance to a prior anti-cancer therapy administered to the individual, the individual is predicted to respond to an anti-cancer therapy, and/or the individual is predicted to have poor prognosis, as compared to an individual whose cancer does not comprise the fusion nucleic acid molecule or fusion polypeptide encoded by the fusion nucleic acid molecule.

Exemplary Clause 11: A method of assessing a fusion nucleic acid molecule or a fusion polypeptide in a cancer in an individual, the method comprising:
(a) detecting in a sample from the individual a fusion nucleic acid molecule, or a fusion polypeptide encoded by the fusion nucleic acid molecule, wherein:
(i) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or
(ii) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2; and
(b) providing an assessment of the fusion nucleic acid molecule or fusion polypeptide encoded by the fusion nucleic acid molecule.

Exemplary Clause 12: A method of detecting a fusion nucleic acid molecule or a fusion polypeptide, the method comprising detecting in a sample from an individual having a cancer a fusion nucleic acid molecule, or a fusion polypeptide encoded by the fusion nucleic acid molecule, wherein:
(a) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or
(b) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2.

Exemplary Clause 13: A method of detecting the presence or absence of a cancer in an individual, the method comprising:
(a) detecting the presence or absence of a cancer in a sample from the individual; and
(b) detecting in a sample from the individual the presence or absence of a fusion nucleic acid molecule, or a fusion polypeptide encoded by the fusion nucleic acid molecule, wherein:
(i) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or
(ii) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2.

Exemplary Clause 14: The method of clause 13, comprising detecting the presence of the cancer in a sample from the individual.

Exemplary Clause 15: The method of clause 13 or clause 14, comprising detecting the presence of the fusion nucleic acid molecule, or the fusion polypeptide encoded by the fusion nucleic acid molecule, in a sample from the individual.

Exemplary Clause 16: A method for monitoring progression or recurrence of a cancer in an individual, the method comprising:
(a) detecting, in a first sample obtained from the individual at a first time point, the presence or absence of a fusion nucleic acid molecule, or a fusion polypeptide encoded by the fusion nucleic acid molecule;
(b) detecting, in a second sample obtained from the individual at a second time point after the first time point, the presence or absence of a fusion nucleic acid molecule, or a fusion polypeptide encoded by the fusion nucleic acid molecule; and
(c) providing an assessment of cancer progression or cancer recurrence in the individual based, at least in part, on the presence or absence of the fusion nucleic acid molecule or fusion polypeptide encoded by the fusion nucleic acid molecule in the first sample and/or in the second sample;
wherein:
(i) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or
(ii) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2.

Exemplary Clause 17: The method of clause 16, wherein the presence of the fusion nucleic acid molecule or fusion polypeptide encoded by the fusion nucleic acid molecule in the first sample and/or in the second sample identifies the individual as having increased risk of cancer progression or cancer recurrence.

Exemplary Clause 18: The method of clause 16 or clause 17, further comprising selecting a treatment, administering a treatment, adjusting a treatment, adjusting the dose of a treatment, or applying a treatment to the individual based, at least in part, on detecting the presence of the fusion nucleic acid molecule or fusion polypeptide encoded by the fusion nucleic acid molecule in the first sample and/or in the second sample, wherein the treatment comprises an anti-cancer therapy.

Exemplary Clause 19: A method of detecting a fusion nucleic acid molecule, the method comprising:
(a) providing a plurality of nucleic acid molecules obtained from a sample from an individual having a cancer, wherein the plurality of nucleic acid molecules comprises nucleic acid molecules corresponding to a fusion nucleic acid molecule, wherein:
(i) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or
(ii) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2;
(b) optionally, ligating one or more adapters onto one or more nucleic acid molecules from the plurality of nucleic acid molecules;
(c) optionally, amplifying the one or more ligated nucleic acid molecules from the plurality of nucleic acid molecules;
(d) optionally, capturing amplified nucleic acid molecules from the amplified nucleic acid molecules;
(e) sequencing, by a sequencer, the captured nucleic acid molecules to obtain a plurality of sequence reads that represent the captured nucleic acid molecules, wherein one or more of the plurality of sequence reads correspond to the fusion nucleic acid molecule;
(f) analyzing the plurality of sequence reads; and
(g) based on the analysis, detecting the presence or absence of the fusion nucleic acid molecule in the sample.

Exemplary Clause 20: The method of clause 19, further comprising receiving, at one or more processors, sequence read data for the plurality of sequence reads.

Exemplary Clause 21: The method of clause 20, wherein the analyzing the plurality of sequence reads comprises identifying, using the one or more processors, the presence or absence of sequence reads corresponding to the fusion nucleic acid molecule.

Exemplary Clause 22: The method of any one of clauses 19-21, wherein the amplified nucleic acid molecules are captured by hybridization with one or more bait molecules.

Exemplary Clause 23: A method of detecting a fusion nucleic acid molecule, the method comprising:
(a) providing a sample from an individual having a cancer, wherein the sample comprises a plurality of nucleic acid molecules;
(b) preparing a nucleic acid sequencing library from the plurality of nucleic acid molecules in the sample;
(c) amplifying said library;
(d) selectively enriching for one or more nucleic acid molecules comprising nucleotide sequences corresponding to a fusion nucleic acid molecule in said library to produce an enriched sample,
wherein:
(i) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or
(ii) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2;
(e) sequencing the enriched sample, thereby producing a plurality of sequence reads;
(f) analyzing the plurality of sequence reads for the presence of the fusion nucleic acid molecule;
(g) detecting, based on the analyzing step, the presence or absence of the fusion nucleic acid molecule in the sample from the individual.

Exemplary Clause 24: The method of any one of clauses 19-23, wherein the plurality of nucleic acid molecules comprises a mixture of cancer nucleic acid molecules and non-cancer nucleic acid molecules.

Exemplary Clause 25: The method of clause 24, wherein the cancer nucleic acid molecules are derived from a tumor portion of a heterogeneous tissue biopsy sample, and the non-cancer nucleic acid molecules are derived from a normal portion of the heterogeneous tissue biopsy sample.

Exemplary Clause 26: The method of clause 24, wherein the sample comprises a liquid biopsy sample, and wherein the cancer nucleic acid molecules are derived from a circulating tumor DNA (ctDNA) fraction of the liquid biopsy sample, and the non-cancer nucleic acid molecules are derived from a non-tumor and/or cell-free DNA (cfDNA) fraction of the liquid biopsy sample.

Exemplary Clause 27: The method of any one of clauses 19-22 and 24-26, wherein the one or more adapters comprise amplification primers, flow cell adaptor sequences, substrate adapter sequences, or sample index sequences.

Exemplary Clause 28: The method of any one of clauses 23-27, wherein the selectively enriching comprises: (a) combining one or more bait molecules with the library, thereby hybridizing the one or more bait molecules to one or more nucleic acid molecules comprising nucleotide sequences corresponding to the fusion nucleic acid molecule and producing nucleic acid hybrids; and (b) isolating the nucleic acid hybrids to produce the enriched sample.

Exemplary Clause 29: The method of any one of clauses 19-22 and 24-27, wherein the captured nucleic acid molecules are captured from the amplified nucleic acid molecules by hybridization to one or more bait molecules.

Exemplary Clause 30: The method of any one of clauses 19-29, wherein the amplifying comprises performing a polymerase chain reaction (PCR) amplification technique, a non-PCR amplification technique, or an isothermal amplification technique.

Exemplary Clause 31: The method of any one of clauses 19-30, wherein the sequencing comprises use of a massively parallel sequencing (MPS) technique, whole genome sequencing (WGS), whole exome sequencing, targeted sequencing, direct sequencing, or a Sanger sequencing technique.

Exemplary Clause 32: The method of clause 31, wherein the sequencing comprises a massively parallel sequencing technique, and the massively parallel sequencing technique comprises next generation sequencing (NGS).

Exemplary Clause 33: The method of any one of clauses 19-22 and 24-32, wherein the sequencer comprises a next generation sequencer.

Exemplary Clause 34: The method of any one of clauses 19-33, further comprising generating a genomic profile for the individual, based, at least in part, on detecting the presence or absence of the fusion nucleic acid molecule.

Exemplary Clause 35: The method of clause 34, wherein the genomic profile for the individual further comprises results from a comprehensive genomic profiling (CGP) test, a gene expression profiling test, a cancer hotspot panel test, a DNA methylation test, a DNA fragmentation test, an RNA fragmentation test, or any combination thereof.

Exemplary Clause 36: The method of clause 34 or clause 35, wherein the genomic profile for the individual further comprises results from a nucleic acid sequencing-based test.

Exemplary Clause 37: The method of any one of clauses 34-36, further comprising selecting a treatment, administering a treatment, or applying a treatment to the individual based on the generated genomic profile, wherein the treatment comprises an anti-cancer therapy.

Exemplary Clause 38: The method of any one of clauses 19-37, further comprising generating a report indicating the presence or absence of the fusion nucleic acid molecule in the sample.

Exemplary Clause 39: The method of clause 21 or clause 22, further comprising generating, by the one or more processors, a report indicating the presence or absence of the fusion nucleic acid molecule in the sample.

Exemplary Clause 40: The method of clause 38 or clause 39, further comprising transmitting the report to a healthcare provider.

Exemplary Clause 41: The method of clause 40, wherein the report is transmitted via a computer network or a peer-to-peer connection.

Exemplary Clause 42: A method of identifying a candidate treatment for a cancer in an individual in need thereof, comprising performing DNA sequencing on a sample obtained from the individual to determine a sequencing mutation profile on a group of genes comprising one or more of ALK, BRAF, EGFR, ERBB2, FGFR1, FGFR2, FGFR3, MET, NTRK1, RAF1, RET, or ROS1, or any combination thereof, wherein the sequencing mutation profile identifies the presence or absence of a fusion nucleic acid molecule, wherein:
(a) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or
(b) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2.

Exemplary Clause 43: The method of clause 42, wherein the candidate treatment comprises an anti-cancer therapy.

Exemplary Clause 44: The method of clause 43, wherein the presence of the fusion nucleic acid molecule in the sample identifies the individual as one who may benefit from a treatment comprising an anti-cancer therapy.

Exemplary Clause 45: The method of clause 43 or clause 44, wherein the presence of the fusion nucleic acid molecule in the sample predicts the individual to have longer survival when treated with a treatment comprising an anti-cancer therapy, as compared to survival of an individual whose cancer does not comprise the fusion nucleic acid molecule.

Exemplary Clause 46: The method of any one of clauses 42-45, wherein the sequencing comprises use of a massively parallel sequencing (MPS) technique, whole genome sequencing (WGS), whole exome sequencing, targeted sequencing, direct sequencing, or a Sanger sequencing technique.

Exemplary Clause 47: The method of clause 46, wherein the sequencing comprises a massively parallel sequencing technique, and the massively parallel sequencing technique comprises next generation sequencing (NGS).

Exemplary Clause 48: The method of any one of clauses 42-47, wherein the sequencing mutation profile identifies the presence or absence of a fragment of the fusion nucleic acid molecule comprising a breakpoint or fusion junction.

Exemplary Clause 49: A method of treating or delaying progression of cancer, comprising:
(a) detecting in a sample from an individual having a cancer a fusion nucleic acid molecule, or a fusion polypeptide encoded by the fusion nucleic acid molecule, wherein:
(i) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or
(ii) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2; and
(b) administering to the individual an effective amount of a treatment that comprises an anti-cancer therapy.

Exemplary Clause 50: The method of any one of clauses 1-49, wherein the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, comprising or resulting from a Breakpoint 1 and/or a Breakpoint 2 corresponding to the ALK, BRAF, EGFR, ERBB2, FGFR1, FGFR2, FGFR3, MET, RAF1, RET, or ROS1 fusion nucleic acid molecule as listed in Table 3.

Exemplary Clause 51: The method of any one of clauses 1-50, wherein the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, and wherein the cancer is a carcinoma, a sarcoma, a lymphoma, a leukemia, a myeloma, a germ cell cancer, or a blastoma.

Exemplary Clause 52: The method of any one of clauses 1-51, wherein the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, and wherein the cancer is a solid tumor.

Exemplary Clause 53: The method of any one of clauses 1-51, wherein the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, and wherein the cancer is a hematologic malignancy.

Exemplary Clause 54: The method of any one of clauses 1-51, wherein the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, and wherein the cancer is a B cell cancer, a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

Exemplary Clause 55: The method of any one of clauses 1-51, wherein the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, and wherein the cancer is appendix adenocarcinoma, bladder adenocarcinoma, bladder urothelial (transitional cell) carcinoma, breast cancer not otherwise specified (NOS), breast carcinoma NOS, breast invasive ductal carcinoma (IDC), breast invasive lobular carcinoma (ILC), cervix squamous cell carcinoma (SCC), colon adenocarcinoma (CRC), esophagus adenocarcinoma, esophagus carcinoma NOS, esophagus squamous cell carcinoma (SCC), eye intraocular melanoma, gallbladder adenocarcinoma, gastroesophageal junction adenocarcinoma, intra-hepatic cholangiocarcinoma, kidney cancer NOS, liver hepatocellular carcinoma (HCC), lung cancer NOS, lung adenocarcinoma, lung large cell carcinoma, lung non-small cell lung carcinoma (NSCLC) NOS, lung small cell undifferentiated carcinoma, lung squamous cell carcinoma (SCC), ovary cancer NOS, pancreas cancer NOS, pancreas ductal adenocarcinoma, pancreatobiliary carcinoma, prostate cancer NOS, prostate acinar adenocarcinoma, prostate ductal adenocarcinoma, rectum adenocarcinoma (CRC), skin melanoma, small intestine adenocarcinoma, soft tissue sarcoma NOS, stomach adenocarcinoma NOS, unknown primary cancer NOS, unknown primary adenocarcinoma, unknown primary carcinoma (CUP) NOS, unknown primary neuroendocrine tumor, unknown primary squamous cell carcinoma (SCC), or uterus endometrial adenocarcinoma NOS.

Exemplary Clause 56: The method of any one of clauses 1-51, wherein the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, and the cancer is the cancer corresponding to the ALK, BRAF, EGFR, ERBB2, FGFR1, FGFR2, FGFR3, MET, RAF1, RET, or ROS1 fusion nucleic acid molecule as listed in Table 4.

Exemplary Clause 57: The method of any one of clauses 1-51, wherein the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, the cancer is the cancer corresponding to the ALK, BRAF, EGFR, ERBB2, FGFR1, FGFR2, FGFR3, MET, RAF1, RET, or ROS1 fusion nucleic acid molecule as listed in Table 5, and the fusion nucleic acid molecule comprises or results from a Breakpoint 1 and/or a Breakpoint 2 corresponding to the ALK, BRAF, EGFR, ERBB2, FGFR1, FGFR2, FGFR3, MET, RAF1, RET, or ROS1 fusion nucleic acid molecule as listed in Table 5.

Exemplary Clause 58: The method of any one of clauses 1-49, wherein:
(i) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2; and
(ii) the fusion nucleic acid molecule comprises or results from a Breakpoint 1 and/or a Breakpoint 2 corresponding to the ALK, BRAF, ERBB2, FGFR1, FGFR2, FGFR3, MET, NTRK1, RAF1, RET, or ROS1 fusion nucleic acid molecule as listed in Table 6.

Exemplary Clause 59: The method of any one of clauses 1-58, wherein the fusion polypeptide encoded by the fusion nucleic acid molecule is oncogenic.

Exemplary Clause 60: The method of any one of clauses 1-59, wherein the fusion polypeptide encoded by the fusion nucleic acid molecule promotes cancer cell survival, angiogenesis, cancer cell proliferation, and any combination thereof.

Exemplary Clause 61: The method of any one of clauses 1-10, 18, 37-41, and 43-60, wherein the anti-cancer therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for cancer comprising the fusion nucleic acid molecule or the fusion polypeptide encoded by the fusion nucleic acid molecule, a treatment for cancer being tested in a clinical trial, a targeted therapy, a treatment being tested in a clinical trial for cancer comprising the fusion nucleic acid molecule or fusion polypeptide encoded by the fusion nucleic acid molecule, or any combination thereof.

Exemplary Clause 62: The method of any one of clauses 1-10, 18, 37-41, and 43-61, wherein the anti-cancer therapy is a kinase inhibitor.

Exemplary Clause 63: The method of clause 62, wherein the kinase inhibitor is a multi-kinase inhibitor or an ALK-, BRAF-, EGFR-, ERBB2-, FGFR1-, FGFR2-, FGFR3-, MET-, RAF1-, NTRK1-, RET-, or ROS1-specific inhibitor.

Exemplary Clause 64: The method of any one of clauses 61-63, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

Exemplary Clause 65: The method of any one of clauses 61-64, wherein the nucleic acid inhibits the expression of the fusion nucleic acid molecule or fusion polypeptide encoded by the fusion nucleic acid molecule.

Exemplary Clause 66: The method of any one of clauses 61-65, wherein the nucleic acid comprises a double-stranded RNA (dsRNA), a small interfering RNA (siRNA), or a small hairpin RNA (shRNA).

Exemplary Clause 67: The method of any one of clauses 1-66, further comprising acquiring knowledge of or detecting in a sample from the individual a base substitution, a short insertion/deletion (indel), a copy number alteration, or a genomic rearrangement in one or more genes.

Exemplary Clause 68: The method of any one of clauses 1-67, wherein the individual has received a prior anti-cancer treatment or is being treated with an anti-cancer treatment.

Exemplary Clause 69: The method of clause 68, wherein the fusion nucleic acid molecule, and/or the fusion polypeptide encoded by the fusion nucleic acid molecule, confers resistance of the cancer to the anti-cancer treatment.

Exemplary Clause 70: The method of clause 68 or clause 69, wherein the anti-cancer treatment is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for cancer being tested in a clinical trial, an immunotherapy, a chemotherapy, a targeted therapy, or any combination thereof.

Exemplary Clause 71: The method of clause 70, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

Exemplary Clause 72: The method of clause 70, wherein the nucleic acid comprises a double-stranded RNA (dsRNA), a small interfering RNA (siRNA), or a small hairpin RNA (shRNA).

Exemplary Clause 73: The method of any one of clauses 1-72, wherein the fusion nucleic acid molecule is an ALK fusion nucleic acid molecule as listed in any of Tables 1-6.

Exemplary Clause 74: The method of clause 73, wherein the ALK fusion nucleic acid molecule encodes an ALK fusion polypeptide.

Exemplary Clause 75: The method of clause 74, wherein the encoded ALK fusion polypeptide comprises an ALK kinase domain, or a fragment of an ALK kinase domain having ALK kinase activity.

Exemplary Clause 76: The method of clause 74 or clause 75, wherein the encoded ALK fusion polypeptide has ALK kinase activity, optionally wherein the ALK kinase activity is constitutive.

Exemplary Clause 77: The method of any one of clauses 74-76, wherein the encoded ALK fusion polypeptide is oncogenic.

Exemplary Clause 78: The method of any one of clauses 74-77, wherein the encoded ALK fusion polypeptide promotes cancer cell survival, angiogenesis, cancer cell proliferation, and any combination thereof.

Exemplary Clause 79: The method of any one of clauses 73-78, further comprising acquiring knowledge of or detecting, in a sample from the individual, the presence of:
(a) a mutation in an EGFR gene; optionally wherein the mutation is a deletion of exon 19 of EGFR or a portion thereof, a mutation resulting in an L858R, R748K, T790M, C797S, and/or D761N amino acid substitution in an encoded EGFR polypeptide, an EGFR gene amplification, or any combination thereof;
(b) a mutation in a BRAF gene; optionally wherein the mutation is a mutation resulting in a V600E amino acid substitution in an encoded BRAF polypeptide;
(c) a mutation in an NRAS gene; optionally wherein the mutation is a mutation resulting in a Q61H amino acid substitution in an encoded NRAS polypeptide;
(d) a mutation in a MET gene; optionally wherein the mutation is a MET gene amplification, a mutation resulting in a D1228H amino acid substitution in an encoded MET polypeptide, or both;
(e) a mutation in an NF1 gene; optionally wherein the mutation is an NF1 truncation;
(f) a wild type KRAS gene, or a mutation in a KRAS gene; optionally wherein the mutation results in a G12V and/or A146P amino acid substitution in an encoded KRAS polypeptide;
(g) a mutation in a MAP2K1 gene; optionally wherein the mutation is a mutation resulting in a I103_K104del mutation in an encoded MAP2K1 polypeptide;
(h) an ALK mutation; optionally wherein the ALK mutation is an ALK resistance mutation, and optionally wherein the ALK resistance mutation results in a G1269A, G1202R, I1171S, I1171T, L1196M, T1151M, S1206Y, 11171N, D1203N, F1174C, L1152R, F1174L, L1198F, C1156Y, T1151_L1152insT, V1180L, G1202L, and/or S1206A amino acid substitution in an encoded ALK polypeptide, or any combination thereof; or any combination of (a)-(h).

Exemplary Clause 80: The method of any one of clauses 73-78, further comprising acquiring knowledge of or detecting, in a sample from the individual, the presence of a mutation in an EGFR gene, optionally wherein the mutation results in an L858R amino acid substitution in an encoded EGFR polypeptide; wherein the ALK fusion nucleic acid molecule is an ALK-PLEKHA7 fusion nucleic acid molecule as listed in Tables 2 or 6.

Exemplary Clause 81: The method of clause 80, wherein the cancer is a non-small cell lung carcinoma (NSCLC).

Exemplary Clause 82: The method of clause 80 or clause 81, wherein the individual was previously treated for cancer with erlotinib, afatinib, and/or osimertinib.

Exemplary Clause 83: The method of clause 82, wherein the individual exhibited a partial response to treatment with erlotinib; and/or wherein the individual exhibited a partial response to treatment with osimertinib.

Exemplary Clause 84: The method of any one of clauses 73-83, wherein the fusion nucleic acid molecule, and/or the fusion polypeptide encoded by the fusion nucleic acid molecule, confers resistance of the cancer to an EGFR-targeted anti-cancer therapy, optionally wherein the EGFR-targeted anti-cancer therapy is a first-, second-, or third-generation EGFR tyrosine kinase inhibitor.

Exemplary Clause 85: The method of any one of clauses 73-83, wherein the fusion nucleic acid molecule, and/or the fusion polypeptide encoded by the fusion nucleic acid molecule, confers resistance of the cancer to an EGFR-targeted anti-cancer therapy, optionally wherein the EGFR-targeted anti-cancer therapy is cetuximab, panitumumab, lapatinib, gefitinib, vandetanib, dacomitinib, icotinib, osimertinib (AZD9291), afatanib, olmutinib, EGF816 (nazartinib), avitinib (AC00010), rociletinib (CO-1686), BMS-690514, YH5448, PF-06747775, ASP8273, PF299804, AP26113, or erlotinib.

Exemplary Clause 86: The method of any one of clauses 73-85, wherein the fusion nucleic acid molecule, and/or the fusion polypeptide encoded by the fusion nucleic acid molecule, confers resistance of the cancer to an NF1-targeted anti-cancer therapy.

Exemplary Clause 87: The method of any one of clauses 73-78, further comprising acquiring knowledge of or detecting, in a sample from the individual, the presence of:
(a) an ALK resistance mutation; optionally wherein the ALK resistance mutation results in a V1180L, I1171N, L1196M, D1203N, or I1171T amino acid substitution in an encoded ALK polypeptide, or any combination thereof; and/or
(b) a mutation in a KRAS gene; optionally wherein the mutation results in a G12V amino acid substitution in an encoded KRAS polypeptide;
wherein the ALK fusion nucleic acid molecule is an ALK-HIP1 fusion nucleic acid molecule as listed in Tables 2 or 6.

Exemplary Clause 88: The method of clause 87, wherein the sample comprises one or more ALK gene mutations that result in a V1180L and I1171N amino acid substitution in an encoded ALK polypeptide; or a D1203N and I1171T amino acid substitution in an encoded ALK polypeptide.

Exemplary Clause 89: The method of clause 88, wherein the sample comprises a mutation in a KRAS gene; optionally wherein the mutation results in a G12V amino acid substitution in an encoded KRAS polypeptide.

Exemplary Clause 90: The method of any one of clauses 87-89, wherein the cancer is an unknown primary carcinoma.

Exemplary Clause 91: The method of any one of clauses 73-90, wherein the anti-cancer therapy is an ALK-targeted therapy.

Exemplary Clause 92: The method of clause 91, wherein the ALK-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for ALK-positive or ALK-rearranged cancer, an ALK-targeted therapy being tested in a clinical trial, a treatment for ALK-positive or ALK-rearranged cancer being tested in a clinical trial, or any combination thereof.

Exemplary Clause 93: The method of clause 91 or clause 92, wherein the ALK-targeted therapy is a kinase inhibitor.

Exemplary Clause 94: The method of any one of clauses 91-93, wherein the ALK-targeted therapy is a tyrosine kinase inhibitor.

Exemplary Clause 95: The method of clause 93 or clause 94, wherein the ALK-targeted therapy is a multi-kinase inhibitor or an ALK-specific inhibitor.

Exemplary Clause 96: The method of any one of clauses 93-95, wherein the kinase inhibitor inhibits a kinase activity of an ALK polypeptide.

Exemplary Clause 97: The method of any one of clauses 91-96, wherein the ALK-targeted therapy comprises one or more of crizotinib, alectinib, ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, TAE684 (NVP-TAE684), CT-707, WX-0593, alkotinib, SIM1803-1A, PLB1003, SAF-189s, PF03446962, TQ-B3101, APG-2449, X-376, CEP-28122, and GSK1838705A.

Exemplary Clause 98: The method of any one of clauses 92-97, wherein the nucleic acid inhibits the expression of the ALK fusion nucleic acid molecule or fusion polypeptide encoded by the fusion nucleic acid molecule.

Exemplary Clause 99: The method of any one of clauses 92-98, wherein the nucleic acid is a double-stranded RNA (dsRNA), a small interfering RNA (siRNA), or a small hairpin RNA (shRNA).

Exemplary Clause 100: The method of any one of clauses 92-99, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

Exemplary Clause 101: The method of any one of clauses 1-72, wherein the fusion nucleic acid molecule is a BRAF fusion nucleic acid molecule as listed in any of Tables 1-6.

Exemplary Clause 102: The method of clause 101, wherein the BRAF fusion nucleic acid molecule encodes a BRAF fusion polypeptide.

Exemplary Clause 103: The method of clause 102, wherein the encoded BRAF fusion polypeptide comprises a BRAF kinase domain, or a fragment of a BRAF kinase domain having BRAF kinase activity.

Exemplary Clause 104: The method of clause 102 or clause 103, wherein the encoded BRAF fusion polypeptide has BRAF kinase activity, optionally wherein the BRAF kinase activity is constitutive.

Exemplary Clause 105: The method of any one of clauses 102-104, wherein the encoded BRAF fusion polypeptide is oncogenic.

Exemplary Clause 106: The method of any one of clauses 102-105, wherein the encoded BRAF fusion polypeptide promotes cancer cell survival, angiogenesis, cancer cell proliferation, and any combination thereof.

Exemplary Clause 107: The method of any one of clauses 101-106, further comprising acquiring knowledge of or detecting, in a sample from the individual, the presence of:
(a) a mutation in an EGFR gene; optionally wherein the mutation is an EGFR gene amplification, and/or a mutation resulting in a V441G, S492R, and/or G465E/R amino acid substitution in an encoded EGFR polypeptide;
(b) a wild type KRAS gene, or a mutation in a KRAS gene; optionally wherein the mutation results in a G12F, G12V, G12C, G13D and/or Q61H amino acid substitution in an encoded KRAS polypeptide;
(c) a mutation in an NRAS gene; optionally wherein the mutation results in a G13D and/or Q61K/L amino acid substitution in an encoded NRAS polypeptide;
(d) a mutation in a MET gene, optionally where the mutation is a MET gene amplification; (e) a mutation in a MAP2K1 gene, optionally wherein the mutation results in a Q58del or E102_I103del mutation and/or I111T or K57T amino acid substitution in an encoded MAP2K1 polypeptide;
(f) a mutation in a MAP2K2 gene, optionally wherein the mutation results in a F57V amino acid substitution in an encoded MAP2K2 polypeptide;
(g) a mutation in an NF1 gene, optionally wherein the mutation is a F945fs*9 mutation;
(h) a mutation in a BRAF gene, optionally wherein the mutation results in a V600E amino acid substitution in an encoded BRAF polypeptide; and/or
(i) a mutation in an HRAS gene, optionally wherein the mutation results in a Q61L amino acid substitution in an encoded HRAS polypeptide.

Exemplary Clause 108: The method of any one of clauses 101-106, further comprising acquiring knowledge of or detecting, in a sample from the individual, the presence of:
an EGFR gene amplification; and
a wild type KRAS gene, or a KRAS gene mutation resulting in a G12F and/or Q61H amino acid substitution in an encoded KRAS polypeptide;
wherein the BRAF fusion nucleic acid molecule is a BRAF-SND1 fusion nucleic acid molecule listed in Tables 2 or 6.

Exemplary Clause 109: The method of any one of clauses 101-106, further comprising acquiring knowledge of or detecting, in a sample from the individual, the presence of:
a mutation in an EGFR gene resulting in a V441G and/or G465E/R amino acid substitution in an encoded EGFR polypeptide;
a wild type KRAS gene, or a KRAS gene mutation resulting in a G12C amino acid substitution in an encoded KRAS polypeptide;
a mutation in an NRAS gene resulting in a G13D and/or Q61K amino acid substitution in an encoded NRAS polypeptide; and
a MET gene amplification,
wherein the BRAF fusion nucleic acid molecule is a BRAF-ZC3HAV1 fusion nucleic acid molecule listed in Tables 2 or 6.

Exemplary Clause 110: The method of any one of clauses 101-106, further comprising acquiring knowledge of or detecting, in a sample from the individual, the presence of:
a mutation in an EGFR gene resulting in an S492R amino acid substitution in an encoded EGFR polypeptide;
a wild type KRAS gene, or a mutation in a KRAS gene; optionally wherein the mutation results in a G12V and/or Q61H amino acid substitution in an encoded KRAS polypeptide;
a mutation in an NRAS gene resulting in a Q61K/L amino acid substitution in an encoded NRAS polypeptide;
a mutation in a MAP2K1 gene resulting in a Q58del mutation and/or I111T amino acid substitution in an encoded MAP2K1 polypeptide;
a mutation in a MAP2K2 gene resulting in a F57V amino acid substitution in an encoded MAP2K2 polypeptide; and
a F945fs*9 mutation in an NF1 gene,
wherein the BRAF fusion nucleic acid molecule is an BRAF-MKRN1 fusion nucleic acid molecule listed in Tables 2 or 6.

Exemplary Clause 111: The method of any one of clauses 101-106, further comprising acquiring knowledge of or detecting, in a sample from the individual, the presence of:
a mutation in a KRAS gene resulting in a G13D amino acid substitution in an encoded KRAS polypeptide;
wherein the BRAF fusion nucleic acid molecule is a BRAF-DENND2A fusion nucleic acid molecule listed in Tables 2 or 6.

Exemplary Clause 112: The method of clause 111, wherein the cancer was previously treated with folinic acid, fluorouracil (5-FU), and oxaliplatin (FOLFOX); 5-FU; folinic acid, 5-FU, and irinotecan (FOLFIRI); and/or regorafenib.

Exemplary Clause 113: The method of any one of clauses 101-106, further comprising acquiring knowledge of or detecting, in a sample from the individual, the presence of:
a wild type KRAS gene; and
a mutation in an NRAS gene resulting in a Q61K amino acid substitution in an encoded NRAS polypeptide;
wherein the BRAF fusion nucleic acid molecule is an BRAF-TRIM24 fusion nucleic acid molecule listed in Tables 2 or 6.

Exemplary Clause 114: The method of any one of clauses 108-113, wherein the cancer is a colorectal cancer.

Exemplary Clause 115: The method of any one of clauses 101-106, further comprising acquiring knowledge of or detecting, in a sample from the individual, the presence of:
a mutation in a BRAF gene resulting in an V600E amino acid substitution in an encoded BRAF polypeptide;
a mutation in an EGFR gene resulting in a S492R and/or V441G amino acid substitution in an encoded EGFR polypeptide;
a wild type KRAS gene;
a mutation in an HRAS gene resulting in an Q61L amino acid substitution in an encoded HRAS polypeptide;
a mutation in a MAP2K1 gene resulting in a E102_I103del mutation and/or a K57T amino acid substitution in an encoded MAP2K1 polypeptide; and
a mutation in an NRAS gene resulting in a Q61K amino acid substitution in an encoded NRAS polypeptide,
wherein the BRAF fusion nucleic acid molecule is a BRAF-GOLGA3 fusion nucleic acid molecule as listed in any of Tables 1 and 3-5.

Exemplary Clause 116: The method of clause 115, wherein the cancer is a colorectal cancer.

Exemplary Clause 117: The method of clause 115 or clause 116, wherein the cancer was previously treated with 5-FU; folinic acid, 5-FU, and irinotecan (FOLFIRI) in combination with bevacizumab; FOLFIRI in combination with cetuximab; folinic acid, 5-FU, and oxaliplatin (FOLFOX) in combination with bevacizumab; and/or pembrolizumab in combination with regorafenib.

Exemplary Clause 118: The method of any one of clauses 101-106, further comprising acquiring knowledge of or detecting, in a sample from the individual, the presence of:
a mutation in a KRAS gene resulting in a G12C and/or G13D amino acid substitution in an encoded KRAS polypeptide;
a mutation in a MAP2K1 gene resulting in a E102_I103del mutation in an encoded MAP2K1 polypeptide; and
a mutation in an NRAS gene resulting in an Q61K amino acid substitution in an encoded NRAS polypeptide,
wherein the BRAF fusion nucleic acid molecule is an BRAF-AKAP9 fusion nucleic acid molecule as listed in Tables 2 or 6.

Exemplary Clause 119: The method of clause 118, wherein the cancer is a colorectal cancer.

Exemplary Clause 120: The method of clause 118 or clause 119, wherein the cancer was previously treated with adagrasib or adagrasib in combination with cetuximab.

Exemplary Clause 121: The method of any one of clauses 101-120, wherein the fusion nucleic acid molecule, and/or the fusion polypeptide encoded by the fusion nucleic acid molecule, confers resistance of the cancer to an EGFR-targeted anti-cancer therapy, optionally wherein the EGFR-targeted anti-cancer therapy is a first-, second-, or third-generation EGFR tyrosine kinase inhibitor.

Exemplary Clause 122: The method of any one of clauses 101-120, wherein the fusion nucleic acid molecule, and/or the fusion polypeptide encoded by the fusion nucleic acid molecule, confers resistance of the cancer to an EGFR-targeted anti-cancer therapy, optionally wherein the EGFR-targeted anti-cancer therapy is cetuximab, panitumumab, lapatinib, gefitinib, vandetanib, dacomitinib, icotinib, osimertinib (AZD9291), afatanib, olmutinib, EGF816 (nazartinib), avitinib (AC00010), rociletinib (CO-1686), BMS-690514, YH5448, PF-06747775, ASP8273, PF299804, AP26113, or erlotinib.

Exemplary Clause 123: The method of any one of clauses 101-122, wherein the anti-cancer therapy is a BRAF-targeted therapy.

Exemplary Clause 124: The method of clause 123, wherein the BRAF-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for BRAF-rearranged cancer, a BRAF-targeted therapy being tested in a clinical trial, a treatment for BRAF-rearranged cancer being tested in a clinical trial, or any combination thereof.

Exemplary Clause 125: The method of clause 123 or clause 124, wherein the BRAF-targeted therapy is a kinase inhibitor.

Exemplary Clause 126: The method of any one of clauses 123-125, wherein the BRAF-targeted therapy is a serine/threonine kinase inhibitor.

Exemplary Clause 127: The method of any one of clauses 123-126, wherein the BRAF-targeted therapy is a multi-kinase inhibitor or a BRAF-specific inhibitor.

Exemplary Clause 128: The method of any one of clauses 125-127, wherein the kinase inhibitor inhibits a kinase activity of a BRAF polypeptide.

Exemplary Clause 129: The method of any one of clauses 123-128, wherein the BRAF-targeted therapy comprises one or more of sorafenib, PLX4720, PLX-3603, dabrafenib (GSK2118436), encorafenib (LGX818), GDC-0879, RAF265, XL281, ARQ736, BAY73-4506, vemurafenib, cobimetinib, binimetinib, regorafenib, selumetinib, trametinib, or BAY 43-9006.

Exemplary Clause 130: The method of any one of clauses 124-129, wherein the nucleic acid inhibits the expression of the BRAF fusion nucleic acid molecule or fusion polypeptide encoded by the fusion nucleic acid molecule.

Exemplary Clause 131: The method of any one of clauses 124-130, wherein the nucleic acid is a double-stranded RNA (dsRNA), a small interfering RNA (siRNA), or a small hairpin RNA (shRNA).

Exemplary Clause 132: The method of any one of clauses 124-131, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

Exemplary Clause 133: The method of any one of clauses 1-72, wherein the fusion nucleic acid molecule is an EGFR fusion nucleic acid molecule as listed in any of Tables 1 and 3-5.

Exemplary Clause 134: The method of clause 133, wherein the EGFR fusion nucleic acid molecule encodes an EGFR fusion polypeptide.

Exemplary Clause 135: The method of clause 134, wherein the encoded EGFR fusion polypeptide comprises an EGFR kinase domain, or a fragment of an EGFR kinase domain having EGFR kinase activity.

Exemplary Clause 136: The method of clause 134 or clause 135, wherein the encoded EGFR fusion polypeptide has EGFR kinase activity, optionally wherein the EGFR kinase activity is constitutive.

Exemplary Clause 137: The method of any one of clauses 134-136, wherein the encoded EGFR fusion polypeptide is oncogenic.

Exemplary Clause 138: The method of any one of clauses 134-137, wherein the encoded EGFR fusion polypeptide promotes cancer cell survival, angiogenesis, cancer cell proliferation, and any combination thereof.

Exemplary Clause 139: The method of any one of clauses 133-138, further comprising acquiring knowledge of or detecting, in a sample from the individual, the presence of:
(a) a wild type KRAS gene, or a mutation in a KRAS gene; optionally wherein the mutation results in a G12A, and/or Q61H amino acid substitution in an encoded KRAS polypeptide;
(b) a mutation in an NRAS gene; optionally wherein the mutation results in a G12D amino acid substitution in an encoded NRAS polypeptide; and/or
(c) a mutation in a MAP2K1 gene, optionally wherein the mutation results in a E102_I103del mutation in an encoded MAP2K1 polypeptide.

Exemplary Clause 140: The method of any one of clauses 133-138, further comprising acquiring knowledge of or detecting, in a sample from the individual, the presence of:
a wild type KRAS gene, or a mutation in a KRAS gene resulting in a G12A, and/or Q61H amino acid substitution in an encoded KRAS polypeptide;
a mutation in an NRAS gene resulting in a G12D amino acid substitution in an encoded NRAS polypeptide; and/or
a mutation in a MAP2K1 gene resulting in a E102_I103del mutation in an encoded MAP2K1 polypeptide,
wherein the fusion nucleic acid molecule is an EGFR-PDE7A fusion nucleic acid molecule listed in any of Tables 1 and 3-5.

Exemplary Clause 141: The method of any one of clauses 133-140, wherein the anti-cancer therapy is an EGFR-targeted therapy.

Exemplary Clause 142: The method of clause 141, wherein the EGFR-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for an EGFR-rearranged cancer, an EGFR-targeted therapy being tested in a clinical trial, a treatment for EGFR-rearranged cancer being tested in a clinical trial, or any combination thereof.

Exemplary Clause 143: The method of clause 141 or clause 142, wherein the EGFR-targeted therapy is a kinase inhibitor.

Exemplary Clause 144: The method of any one of clauses 141-143, wherein the EGFR-targeted therapy is a tyrosine kinase inhibitor.

Exemplary Clause 145: The method of any one of clauses 141-144, wherein the EGFR-targeted therapy is a multi-kinase inhibitor or an EGFR-specific inhibitor.

Exemplary Clause 146: The method of any one of clauses 143-145, wherein the kinase inhibitor inhibits a kinase activity of an EGFR polypeptide.

Exemplary Clause 147: The method of any one of clauses 141-146, wherein the EGFR-targeted therapy comprises one or more of cetuximab, panitumumab, lapatinib, gefitinib, vandetanib, dacomitinib, icotinib, osimertinib (AZD9291), afatanib, olmutinib, EGF816 (nazartinib), avitinib (AC00010), rociletinib (CO-1686), BMS-690514, YH5448, PF-06747775, ASP8273, PF299804, AP26113, necitumumab, or erlotinib.

Exemplary Clause 148: The method of any one of clauses 142-147, wherein the nucleic acid inhibits the expression of the EGFR fusion nucleic acid molecule or fusion polypeptide encoded by the fusion nucleic acid molecule.

Exemplary Clause 149: The method of any one of clauses 142-148, wherein the nucleic acid is a double-stranded RNA (dsRNA), a small interfering RNA (siRNA), or a small hairpin RNA (shRNA).

Exemplary Clause 150: The method of any one of clauses 142-149, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

Exemplary Clause 151: The method of any one of clauses 1-72, wherein the fusion nucleic acid molecule is an ERBB2 fusion nucleic acid molecule as listed in any of Tables 1-6.

Exemplary Clause 152: The method of clause 151, wherein the ERBB2 fusion nucleic acid molecule encodes an ERBB2 fusion polypeptide.

Exemplary Clause 153: The method of clause 152, wherein the encoded ERBB2 fusion polypeptide comprises an ERBB2 kinase domain, or a fragment of an ERBB2 kinase domain having ERBB2 kinase activity.

Exemplary Clause 154: The method of clause 152 or clause 153, wherein the encoded ERBB2 fusion polypeptide has ERBB2 kinase activity, optionally wherein the ERBB2 kinase activity is constitutive.

Exemplary Clause 155: The method of any one of clauses 152-154, wherein the encoded ERBB2 fusion polypeptide is oncogenic.

Exemplary Clause 156: The method of any one of clauses 152-155, wherein the encoded ERBB2 fusion polypeptide promotes cancer cell survival, angiogenesis, cancer cell proliferation, and any combination thereof.

Exemplary Clause 157: The method of any one of clauses 151-156, wherein the anti-cancer therapy is an ERBB2-targeted therapy.

Exemplary Clause 158: The method of clause 157, wherein the ERBB2-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for an ERBB2-rearranged cancer, an ERBB2-targeted therapy being tested in a clinical trial, a treatment for ERBB2-rearranged cancer being tested in a clinical trial, or any combination thereof.

Exemplary Clause 159: The method of clause 157 or clause 158, wherein the ERBB2-targeted therapy is a kinase inhibitor.

Exemplary Clause 160: The method of any one of clauses 157-159, wherein the ERBB2-targeted therapy is a tyrosine kinase inhibitor.

Exemplary Clause 161: The method of any one of clauses 157-160, wherein the ERBB2-targeted therapy is a multi-kinase inhibitor or an ERBB2-specific inhibitor.

Exemplary Clause 162: The method of any one of clauses 159-161, wherein the kinase inhibitor inhibits a kinase activity of an ERBB2 polypeptide.

Exemplary Clause 163: The method of any one of clauses 157-162, wherein the ERBB2-targeted therapy comprises one or more of afatinib, TAK-285, neratinib, dacomitinib, BMS-690514, BMS-599626, pelitinib, CP-724714, lapatinib, TAK-165, ARRY-380, AZD8931, AV-203, AMG-888, MM-111, MM-121, MM-141, LJM716, REGN1400, MEHD7945A, RG7116, trastuzumab, trastuzumab emtansine (T-DM1), pertuzumab, or APC 8024.

Exemplary Clause 164: The method of any one of clauses 158-163, wherein the nucleic acid inhibits the expression of the ERBB2 fusion nucleic acid molecule or fusion polypeptide encoded by the fusion nucleic acid molecule.

Exemplary Clause 165: The method of any one of clauses 158-164, wherein the nucleic acid is a double-stranded RNA (dsRNA), a small interfering RNA (siRNA), or a small hairpin RNA (shRNA).

Exemplary Clause 166: The method of any one of clauses 158-165, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

Exemplary Clause 167: The method of any one of clauses 1-72, wherein the fusion nucleic acid molecule is an FGFR1 fusion nucleic acid molecule as listed in any of Tables 1-6.

Exemplary Clause 168: The method of clause 167, wherein the FGFR1 fusion nucleic acid molecule encodes an FGFR1 fusion polypeptide.

Exemplary Clause 169: The method of clause 168, wherein the encoded FGFR1 fusion polypeptide comprises an FGFR1 kinase domain, or a fragment of an FGFR1 kinase domain having FGFR1 kinase activity.

Exemplary Clause 170: The method of clause 168 or clause 169, wherein the encoded FGFR1 fusion polypeptide has FGFR1 kinase activity, optionally wherein the FGFR1 kinase activity is constitutive.

Exemplary Clause 171: The method of any one of clauses 168-170, wherein the encoded FGFR1 fusion polypeptide is oncogenic.

Exemplary Clause 172: The method of any one of clauses 168-171, wherein the encoded FGFR1 fusion polypeptide promotes cancer cell survival, angiogenesis, cancer cell proliferation, and any combination thereof.

Exemplary Clause 173: The method of any one of clauses 167-172, wherein the anti-cancer therapy is an FGFR1-targeted therapy.

Exemplary Clause 174: The method of clause 173, wherein the FGFR1-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for an FGFR1-rearranged cancer, an FGFR1-targeted therapy being tested in a clinical trial, a treatment for FGFR1-rearranged cancer being tested in a clinical trial, or any combination thereof.

Exemplary Clause 175: The method of clause 173 or clause 174, wherein the FGFR1-targeted therapy is a kinase inhibitor.

Exemplary Clause 176: The method of any one of clauses 173-175, wherein the FGFR1-targeted therapy is a tyrosine kinase inhibitor.

Exemplary Clause 177: The method of any one of clauses 173-176, wherein the FGFR1-targeted therapy is a multi-kinase inhibitor or an FGFR1-specific inhibitor.

Exemplary Clause 178: The method of any one of clauses 175-177, wherein the kinase inhibitor inhibits a kinase activity of an FGFR1 polypeptide.

Exemplary Clause 179: The method of any one of clauses 173-178, wherein the FGFR1-targeted therapy comprises one or more of E3810 (lucitanib), AZD4547, Dovitinib (TKI258), Ponatinib, Derazantinib (ARQ 087), Nintendanib (BIBF1120), Rogaratinib (BAY 1163877), 3D185, SOMCL-085, brivanib (BMS582664), lenvatinib (E7080), orantinib (TSU-68), PRN1371, XL-228, AZ12908010 (AZ8010), Debio-1347 (CH5183284), FIIN-2, LY2874455, Infigratinib (BGJ398, NVP-BGJ398), Pemigatinib, Erdafitinib (JNJ-42756493), ASP5878, TAS-120, PRN1371, pazopanib, regorafenib, or PKC412.

Exemplary Clause 180: The method of any one of clauses 174-179, wherein the nucleic acid inhibits the expression of the FGFR1 fusion nucleic acid molecule or fusion polypeptide encoded by the fusion nucleic acid molecule.

Exemplary Clause 181: The method of any one of clauses 174-180, wherein the nucleic acid is a double-stranded RNA (dsRNA), a small interfering RNA (siRNA), or a small hairpin RNA (shRNA).

Exemplary Clause 182: The method of any one of clauses 174-181, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

Exemplary Clause 183: The method of any one of clauses 1-72, wherein the fusion nucleic acid molecule is an FGFR2 fusion nucleic acid molecule as listed in any of Tables 1-6.

Exemplary Clause 184: The method of clause 183, wherein the FGFR2 fusion nucleic acid molecule encodes an FGFR2 fusion polypeptide.

Exemplary Clause 185: The method of clause 184, wherein the encoded FGFR2 fusion polypeptide comprises an FGFR2 kinase domain, or a fragment of an FGFR2 kinase domain having FGFR2 kinase activity.

Exemplary Clause 186: The method of clause 184 or clause 185, wherein the encoded FGFR2 fusion polypeptide has FGFR2 kinase activity, optionally wherein the FGFR2 kinase activity is constitutive.

Exemplary Clause 187: The method of any one of clauses 184-186, wherein the encoded FGFR2 fusion polypeptide is oncogenic.

Exemplary Clause 188: The method of any one of clauses 184-187, wherein the encoded FGFR2 fusion polypeptide promotes cancer cell survival, angiogenesis, cancer cell proliferation, and any combination thereof.

Exemplary Clause 189: The method of any one of clauses 183-188, further comprising acquiring knowledge of or detecting, in a sample from the individual, the presence of an EGFR gene mutation; optionally wherein the EGFR gene mutation results in an L858R, L833V, and/or T790M amino acid substitution in an encoded EGFR polypeptide.

Exemplary Clause 190: The method of clause 189, wherein the individual has been previously treated for cancer with erlotinib.

Exemplary Clause 191: The method of any one of clauses 183-190, wherein the fusion nucleic acid molecule, and/or the fusion polypeptide encoded by the fusion nucleic acid molecule, confers resistance of the cancer to an EGFR-targeted anti-cancer therapy, optionally wherein the EGFR-targeted anti-cancer therapy is a first-, second-, or third-generation EGFR tyrosine kinase inhibitor.

Exemplary Clause 192: The method of any one of clauses 183-190, wherein the fusion nucleic acid molecule, and/or the encoded fusion polypeptide, confers resistance to an EGFR-targeted anti-cancer therapy, optionally wherein the EGFR-targeted anti-cancer therapy is cetuximab, panitumumab, lapatinib, gefitinib, vandetanib, dacomitinib, icotinib, osimertinib (AZD9291), afatanib, olmutinib, EGF816 (nazartinib), avitinib (AC00010), rociletinib (CO-1686), BMS-690514, YH5448, PF-06747775, ASP8273, PF299804, AP26113, or erlotinib.

Exemplary Clause 193: The method of any one of clauses 183-192, wherein the anti-cancer therapy is an FGFR2-targeted therapy.

Exemplary Clause 194: The method of clause 193, wherein the FGFR2-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for an FGFR2-rearranged cancer, an FGFR2-targeted therapy being tested in a clinical trial, a treatment for FGFR2-rearranged cancer being tested in a clinical trial, or any combination thereof.

Exemplary Clause 195: The method of clause 193 or clause 194, wherein the FGFR2-targeted therapy is a kinase inhibitor.

Exemplary Clause 196: The method of any one of clauses 193-195, wherein the FGFR2-targeted therapy is a tyrosine kinase inhibitor.

Exemplary Clause 197: The method of any one of clauses 193-196, wherein the FGFR2-targeted therapy is a multi-kinase inhibitor or an FGFR2-specific inhibitor.

Exemplary Clause 198: The method of any one of clauses 195-197, wherein the kinase inhibitor inhibits a kinase activity of an FGFR2 polypeptide.

Exemplary Clause 199: The method of any one of clauses 193-198, wherein the FGFR2-targeted therapy comprises one or more of E3810 (lucitanib), AZD4547, Dovitinib (TKI258), Ponatinib, Derazantinib (ARQ 087), Nintendanib (BIBF1120), Rogaratinib (BAY 1163877), 3D185, SOMCL-085, brivanib (BMS582664), lenvatinib (E7080), orantinib (TSU-68), PRN1371, XL-228, AZ12908010 (AZ8010), Debio-1347 (CH5183284), FIIN-2, LY2874455, Infigratinib (BGJ398, NVP-BGJ398), Pemigatinib, Erdafitinib, ASP5878, TAS-120, PRN1371, formononetin, R04383596, Ki23057, SU5402, RLY-4008, pazopanib, regorafenib, or PKC412.

Exemplary Clause 200: The method of any one of clauses 194-199, wherein the nucleic acid inhibits the expression of the FGFR2 fusion nucleic acid molecule or fusion polypeptide encoded by the fusion nucleic acid molecule.

Exemplary Clause 201: The method of any one of clauses 194-200, wherein the nucleic acid is a double-stranded RNA (dsRNA), a small interfering RNA (siRNA), or a small hairpin RNA (shRNA).

Exemplary Clause 202: The method of any one of clauses 194-201, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

Exemplary Clause 203: The method of any one of clauses 1-72, wherein the fusion nucleic acid molecule is an FGFR3 fusion nucleic acid molecule as listed in any of Tables 1-6.

Exemplary Clause 204: The method of clause 203, wherein the FGFR3 fusion nucleic acid molecule encodes an FGFR3 fusion polypeptide.

Exemplary Clause 205: The method of clause 204, wherein the encoded FGFR3 fusion polypeptide comprises an FGFR3 kinase domain, or a fragment of an FGFR3 kinase domain having FGFR3 kinase activity.

Exemplary Clause 206: The method of clause 204 or clause 205, wherein the encoded FGFR3 fusion polypeptide has FGFR3 kinase activity, optionally wherein the FGFR3 kinase activity is constitutive.

Exemplary Clause 207: The method of any one of clauses 204-206, wherein the encoded FGFR3 fusion polypeptide is oncogenic.

Exemplary Clause 208: The method of any one of clauses 204-207, wherein the encoded FGFR3 fusion polypeptide promotes cancer cell survival, angiogenesis, cancer cell proliferation, and any combination thereof.

Exemplary Clause 209: The method of any one of clauses 203-208, further comprising acquiring knowledge of or detecting, in a sample from the individual, the presence of:
(a) a mutation in an EGFR gene; optionally wherein the mutation is a deletion of exon 19 of EGFR or a portion thereof, an EGFR gene amplification, or a mutation resulting in a T790M, C797G, V441G, G465R, E709K or L858R amino acid substitution in an encoded EGFR polypeptide, or any combination thereof;
(b) a mutation in a BRAF gene; optionally wherein the mutation results in a V600E amino acid substitution in an encoded BRAF polypeptide;
(c) a wild type KRAS gene, or a mutation in a KRAS gene; optionally wherein the mutation results in a Q61H amino acid substitution in an encoded KRAS polypeptide;
(d) a mutation in an ESR1 gene; optionally wherein the mutation results in a Y537N and/or D538G amino acid substitution in an encoded ESR1 polypeptide;
(e) a mutation in an AKT1 gene; optionally wherein the mutation results in an E17K amino acid substitution in an encoded AKT1 polypeptide; or any combination of (a)-(e).

Exemplary Clause 210: The method of any one of clauses 203-208, further comprising acquiring knowledge of or detecting, in a sample from the individual, the presence of:
(a) a mutation in an EGFR gene; optionally wherein the mutation is a deletion of exon 19 of EGFR or a portion thereof, an EGFR gene amplification, or a mutation resulting in a S492R, V441G, G465R, E709K or L858R amino acid substitution in an encoded EGFR polypeptide, or any combination thereof;
(b) a wild type KRAS gene, or a mutation in a KRAS gene; optionally wherein the mutation results in a G12C, G13D, and/or Q61H amino acid substitution in an encoded KRAS polypeptide;
(c) a mutation in an ESR1 gene; optionally wherein the mutation results in a Y537N and/or D538G amino acid substitution in an encoded ESR1 polypeptide;
(d) a mutation in an AKT1 gene; optionally wherein the mutation results in an E17K amino acid substitution in an encoded AKT1 polypeptide;
(e) a mutation in a BRAF gene; optionally wherein the mutation results in an V600E amino acid substitution in an encoded BRAF polypeptide;
(f) a mutation in an HRAS gene; optionally wherein the mutation results in an Q61L amino acid substitution in an encoded HRAS polypeptide;
(g) a mutation in a MAP2K1 gene; optionally wherein the mutation results in a E102_I103del mutation and/or a K57T amino acid substitution in an encoded MAP2K1 polypeptide;
(h) a mutation in an NRAS gene; optionally wherein the mutation results in an Q61K amino acid substitution in an encoded NRAS polypeptide; or any combination of (a)-(h);
wherein the FGFR3 fusion nucleic acid molecule is an FGFR3-TACC3 fusion nucleic acid molecule as listed in Tables 2 or 6.

Exemplary Clause 211: The method of clause 210, wherein the cancer is a colorectal cancer, a non-small cell lung cancer, or a breast cancer.

Exemplary Clause 212: The method of clause 210 or clause 211, wherein the sample comprises a deletion of exon 19 of EGFR or a portion thereof.

Exemplary Clause 213: The method of clause 210 or clause 211, wherein the sample comprises EGFR gene mutations resulting in an L858R and/or E709K amino acid substitution in an encoded EGFR polypeptide.

Exemplary Clause 214: The method of any one of clauses 210-213, wherein the individual was previously treated for cancer with afatinib and/or cetuximab.

Exemplary Clause 215: The method of clause 214, wherein the individual experienced stable disease during or after treatment with afatinib and cetuximab.

Exemplary Clause 216: The method of any one of clauses 203-208, further comprising acquiring knowledge of or detecting, in a sample from the individual, the presence of:
a mutation in a BRAF gene resulting in an V600E amino acid substitution in an encoded BRAF polypeptide;
a mutation in an EGFR gene resulting in a S492R and/or V441G amino acid substitution in an encoded EGFR polypeptide;
a wild type KRAS gene;
a mutation in an HRAS gene resulting in an Q61L amino acid substitution in an encoded HRAS polypeptide;
a mutation in a MAP2K1 gene resulting in a E102_I103del mutation and/or a K57T amino acid substitution in an encoded MAP2K1 polypeptide; and
a mutation in an NRAS gene resulting in an Q61K amino acid substitution in an encoded NRAS polypeptide,
wherein the FGFR3 fusion nucleic acid molecule is an FGFR3-TACC3 fusion nucleic acid molecule as listed in Tables 2 or 6.

Exemplary Clause 217: The method of clause 216, wherein the cancer is a colorectal cancer.

Exemplary Clause 218: The method of clause 216 or clause 217, wherein the cancer was previously treated with 5-FU; folinic acid, 5-FU, and irinotecan (FOLFIRI) in combination with bevacizumab; FOLFIRI in combination with cetuximab; folinic acid, 5-FU, and oxaliplatin (FOLFOX) in combination with bevacizumab; and/or pembrolizumab in combination with regorafenib.

Exemplary Clause 219: The method of any one of clauses 203-208, further comprising acquiring knowledge of or detecting, in a sample from the individual, the presence of:
a mutation in a KRAS gene resulting in a G12C and/or G13D amino acid substitution in an encoded KRAS polypeptide;
a mutation in a MAP2K1 gene resulting in a E102_I103del mutation in an encoded MAP2K1 polypeptide; and
a mutation in an NRAS gene resulting in a Q61K amino acid substitution in an encoded NRAS polypeptide,
wherein the FGFR3 fusion nucleic acid molecule is an FGFR3-TACC3 fusion nucleic acid molecule as listed in Tables 2 or 6.

Exemplary Clause 220: The method of clause 219, wherein the cancer is a colorectal cancer.

Exemplary Clause 221: The method of clause 219 or clause 220, wherein the cancer was previously treated with adagrasib or adagrasib in combination with cetuximab.

Exemplary Clause 222: The method of clause 211, wherein the sample comprises an EGFR gene amplification, EGFR gene mutations resulting in a V441G and/or G465R amino acid substitution in an encoded EGFR polypeptide, and a KRAS gene mutation resulting in a Q61H amino acid substitution in an encoded KRAS polypeptide, and wherein the cancer is a colorectal cancer.

Exemplary Clause 223: The method of clause 211, wherein the sample comprises an EGFR gene amplification, EGFR gene mutations resulting in a V441G and/or G465R amino acid substitution in an encoded EGFR polypeptide, and a wild type KRAS gene, and wherein the cancer is a colorectal cancer.

Exemplary Clause 224: The method of clause 222 or clause 223, wherein the individual was previously treated for cancer with FOLFOXIRI (fluorouracil, leucovorin, oxaliplatin, and irinotecan), bevacizumab, and/or panitumumab.

Exemplary Clause 225: The method of any one of clauses 222-224, further comprising acquiring knowledge of or detecting, in a sample from the individual, the presence of an SNRNP70-MET gene fusion.

Exemplary Clause 226: The method of clause 211, wherein the sample comprises ESR1 gene mutations resulting in a Y537N and/or D538G amino acid substitution in an encoded ESR1 polypeptide, and AKT1 gene mutations resulting in an E17K amino acid substitution in an encoded AKT1 polypeptide, and wherein the cancer is a breast cancer.

Exemplary Clause 227: The method of clause 226, wherein the cancer is estrogen receptor-positive (ER+) and/or progesterone receptor-positive (PR+).

Exemplary Clause 228: The method of clause 226 or clause 227, wherein the cancer was previously treated with everolimus, denosumab, and/or fulvestrant.

Exemplary Clause 229: The method of any one of clauses 203-228, wherein the fusion nucleic acid molecule, and/or the fusion polypeptide encoded by the fusion nucleic acid molecule, confers resistance of the cancer to hormonal anti-cancer therapy.

Exemplary Clause 230: The method of any one of clauses 203-208, further comprising acquiring knowledge of or detecting, in a sample from the individual, the presence of
(a) a mutation in an EGFR gene; optionally wherein the mutation is a deletion of exon 19 of EGFR or a portion thereof, or a mutation resulting in a T790M and/or C797G amino acid substitution in an encoded EGFR polypeptide, or any combination thereof;
(b) a mutation in a BRAF gene; optionally wherein the mutation results in a V600E amino acid substitution in an encoded BRAF polypeptide; or both (a) and (b);
wherein the FGFR3 fusion nucleic acid molecule is an FGFR3-ADD1 fusion nucleic acid molecule as listed in Tables 2 or 6.

Exemplary Clause 231: The method of clause 230, wherein the cancer is a non-small cell lung carcinoma (NSCLC).

Exemplary Clause 232: The method of clause 231, wherein the sample comprises a deletion of exon 19 of EGFR or a portion thereof, an EGFR gene mutation resulting in a T790M and/or C797G amino acid substitution in an encoded EGFR polypeptide, and a BRAF gene mutation resulting in a V600E amino acid substitution in an encoded BRAF polypeptide.

Exemplary Clause 233: The method of clause 232, wherein the individual was previously treated for cancer with osimertinib.

Exemplary Clause 234: The method of clause 233, wherein the individual experienced stable disease during or after treatment with osimertinib.

Exemplary Clause 235: The method of any one of clauses 203-234, wherein the fusion nucleic acid molecule, and/or the fusion polypeptide encoded by the fusion nucleic acid molecule, confers resistance to an EGFR-targeted anti-cancer therapy, optionally wherein the EGFR-targeted anti-cancer therapy is a first-, second-, or third-generation EGFR tyrosine kinase inhibitor.

Exemplary Clause 236: The method of any one of clauses 203-234, wherein the fusion nucleic acid molecule, and/or the fusion polypeptide encoded by the fusion nucleic acid molecule, confers resistance of the cancer to an EGFR-targeted anti-cancer therapy, optionally wherein the EGFR-targeted anti-cancer therapy is cetuximab, panitumumab, lapatinib, gefitinib, vandetanib, dacomitinib, icotinib, osimertinib (AZD9291), afatanib, olmutinib, EGF816 (nazartinib), avitinib (AC0010), rociletinib (CO-1686), BMS-690514, YH5448, PF-06747775, ASP8273, PF299804, AP26113, or erlotinib.

Exemplary Clause 237: The method of any one of clauses 203-236, wherein the anti-cancer therapy is an FGFR3-targeted therapy.

Exemplary Clause 238: The method of clause 237, wherein the FGFR3-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for an FGFR3-rearranged cancer, an FGFR3-targeted therapy being tested in a clinical trial, a treatment for FGFR3-rearranged cancer being tested in a clinical trial, or any combination thereof.

Exemplary Clause 239: The method of clause 237 or clause 238, wherein the FGFR3-targeted therapy is a kinase inhibitor.

Exemplary Clause 240: The method of any one of clauses 237-239, wherein the FGFR3-targeted therapy is a tyrosine kinase inhibitor.

Exemplary Clause 241: The method of any one of clauses 237-240, wherein the FGFR3-targeted therapy is a multi-kinase inhibitor or an FGFR3-specific inhibitor.

Exemplary Clause 242: The method of any one of clauses 239-241, wherein the kinase inhibitor inhibits a kinase activity of an FGFR3 polypeptide.

Exemplary Clause 243: The method of any one of clauses 237-242, wherein the FGFR3-targeted therapy comprises one or more of E3810 (lucitanib), AZD4547, Dovitinib (TKI258), Ponatinib, Derazantinib (ARQ 087), Nintendanib (BIBF1120), Rogaratinib (BAY 1163877), 3D185, SOMCL-085, brivanib (BMS582664), lenvatinib (E7080), orantinib (TSU-68), PRN1371, XL-228, AZ12908010 (AZ8010), Debio-1347 (CH5183284), FIIN-2, LY2874455, Infigratinib (BGJ398, NVP-BGJ398), Pemigatinib, Erdafitinib, ASP5878, TAS-120, PRN1371, PKC412, Vofatamab (B-70), pazopanib, or MFGR1877S.

Exemplary Clause 244: The method of any one of clauses 238-243, wherein the nucleic acid inhibits the expression of the FGFR3 fusion nucleic acid molecule or fusion polypeptide encoded by the fusion nucleic acid molecule.

Exemplary Clause 245: The method of any one of clauses 238-244, wherein the nucleic acid is a double-stranded RNA (dsRNA), a small interfering RNA (siRNA), or a small hairpin RNA (shRNA).

Exemplary Clause 246: The method of any one of clauses 238-245, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

Exemplary Clause 247: The method of any one of clauses 1-72, wherein the fusion nucleic acid molecule is a MET fusion nucleic acid molecule as listed in any of Tables 1-6.

Exemplary Clause 248: The method of clause 247, wherein the MET fusion nucleic acid molecule encodes a MET fusion polypeptide.

Exemplary Clause 249: The method of clause 248, wherein the encoded MET fusion polypeptide comprises a MET kinase domain, or a fragment of a MET kinase domain having MET kinase activity.

Exemplary Clause 250: The method of clause 248 or clause 249, wherein the encoded MET fusion polypeptide has MET kinase activity, optionally wherein the MET kinase activity is constitutive.

Exemplary Clause 251: The method of any one of clauses 248-250, wherein the encoded MET fusion polypeptide is oncogenic.

Exemplary Clause 252: The method of any one of clauses 248-251, wherein the encoded MET fusion polypeptide promotes cancer cell survival, angiogenesis, cancer cell proliferation, and any combination thereof.

Exemplary Clause 253: The method of any one of clauses 247-252, further comprising acquiring knowledge of or detecting, in a sample from the individual, the presence of:
(a) a mutation in an EGFR gene; optionally wherein the mutation is an EGFR gene amplification, or a mutation resulting in a V441G and/or G465R amino acid substitution in an encoded EGFR polypeptide, or any combination thereof; and/or
(b) a wild type KRAS gene, or a mutation in a KRAS gene; optionally wherein the mutation results in a Q61H amino acid substitution in an encoded KRAS polypeptide.

Exemplary Clause 254: The method of any one of clauses 247-252, further comprising acquiring knowledge of or detecting, in a sample from the individual, the presence of:
an EGFR gene amplification; EGFR gene mutations resulting in a V441G and/or G465R amino acid substitution in an encoded EGFR polypeptide; and
a wild type KRAS gene, or a KRAS gene mutation resulting in a Q61H amino acid substitution in an encoded KRAS polypeptide;
wherein the MET fusion nucleic acid molecule is a MET-SNRNP70 fusion nucleic acid molecule listed in any of Tables 1 and 3-5.

Exemplary Clause 255: The method of clause 254, further comprising acquiring knowledge of or detecting, in a sample from the individual, the presence of an FGFR3-TACC3 gene fusion.

Exemplary Clause 256: The method of clause 254 or clause 255, wherein the individual was previously treated for cancer with FOLFOXIRI (fluorouracil, leucovorin, oxaliplatin, and irinotecan), bevacizumab, and/or panitumumab.

Exemplary Clause 257: The method of any one of clauses 247-252, further comprising acquiring knowledge of or detecting, in a sample from the individual, the presence of an EGFR gene amplification, and a wild type KRAS gene, or a KRAS gene mutation resulting in a Q61H amino acid substitution in an encoded KRAS polypeptide; wherein the MET fusion nucleic acid molecule is a MET-CAPZA2 fusion nucleic acid molecule listed in Tables 2 or 6.

Exemplary Clause 258: The method of any one of clauses 254-257, wherein the cancer is a colorectal cancer.

Exemplary Clause 259: The method of any one of clauses 247-258, wherein the fusion nucleic acid molecule, and/or the fusion polypeptide encoded by the fusion nucleic acid molecule, confers resistance of the cancer to an EGFR-targeted anti-cancer therapy, optionally wherein the EGFR-targeted anti-cancer therapy is a first-, second-, or third-generation EGFR tyrosine kinase inhibitor.

Exemplary Clause 260: The method of any one of clauses 247-258, wherein the fusion nucleic acid molecule, and/or the fusion polypeptide encoded by the fusion nucleic acid molecule, confers resistance of the cancer to an EGFR-targeted anti-cancer therapy, optionally wherein the EGFR-targeted anti-cancer therapy is cetuximab, panitumumab, lapatinib, gefitinib, vandetanib, dacomitinib, icotinib, osimertinib (AZD9291), afatanib, olmutinib, EGF816 (nazartinib), avitinib (AC00010), rociletinib (CO-1686), BMS-690514, YH5448, PF-06747775, ASP8273, PF299804, AP26113, or erlotinib.

Exemplary Clause 261: The method of any one of clauses 247-260, wherein the anti-cancer therapy is a MET-targeted therapy.

Exemplary Clause 262: The method of clause 261, wherein the MET-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for a MET-rearranged cancer, a MET-targeted therapy being tested in a clinical trial, a treatment for MET-rearranged cancer being tested in a clinical trial, or any combination thereof.

Exemplary Clause 263: The method of clause 261 or clause 262, wherein the MET-targeted therapy is a kinase inhibitor.

Exemplary Clause 264: The method of any one of clauses 261-263, wherein the MET-targeted therapy is a tyrosine kinase inhibitor.

Exemplary Clause 265: The method of any one of clauses 261-264, wherein the MET-targeted therapy is a multi-kinase inhibitor or a MET-specific inhibitor.

Exemplary Clause 266: The method of any one of clauses 263-265, wherein the kinase inhibitor inhibits a kinase activity of a MET polypeptide.

Exemplary Clause 267: The method of any one of clauses 261-266, wherein the MET-targeted therapy comprises PHA-665752, crizotinib, cabozantinib, or capmatinib (INC280).

Exemplary Clause 268: The method of any one of clauses 262-267, wherein the nucleic acid inhibits the expression of the MET fusion nucleic acid molecule or fusion polypeptide encoded by the fusion nucleic acid molecule.

Exemplary Clause 269: The method of any one of clauses 262-268, wherein the nucleic acid is a double-stranded RNA (dsRNA), a small interfering RNA (siRNA), or a small hairpin RNA (shRNA).

Exemplary Clause 270: The method of any one of clauses 262-269, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

Exemplary Clause 271: The method of any one of clauses 1-72, wherein the fusion nucleic acid molecule is a RAF1 fusion nucleic acid molecule as listed in any of Tables 1-6.

Exemplary Clause 272: The method of clause 271, wherein the RAF1 fusion nucleic acid molecule encodes a RAF1 fusion polypeptide.

Exemplary Clause 273: The method of clause 272, wherein the encoded RAF1 fusion polypeptide comprises a RAF1 kinase domain, or a fragment of a RAF1 kinase domain having RAF1 kinase activity.

Exemplary Clause 274: The method of clause 272 or clause 273, wherein the encoded RAF1 fusion polypeptide has RAF1 kinase activity, optionally wherein the RAF1 kinase activity is constitutive.

Exemplary Clause 275: The method of any one of clauses 272-274, wherein the encoded RAF1 fusion polypeptide is oncogenic.

Exemplary Clause 276: The method of any one of clauses 272-275, wherein the encoded RAF1 fusion polypeptide promotes cancer cell survival, angiogenesis, cancer cell proliferation, and any combination thereof.

Exemplary Clause 277: The method of any one of clauses 271-276, further comprising acquiring knowledge of or detecting, in a sample from the individual, the presence of:
(a) a mutation in a BRAF gene, optionally wherein the mutation results in a V600E amino acid substitution in an encoded BRAF polypeptide;
(b) a mutation in an EGFR gene, optionally wherein the mutation results in a S492R and/or V441G amino acid substitution in an encoded EGFR polypeptide;
(c) a wild type KRAS gene, or a mutation in a KRAS gene, optionally wherein the mutation results in a G12C and/or G13D amino acid substitution in an encoded KRAS polypeptide;
(d) a mutation in an HRAS gene, optionally wherein the mutation results in a Q61L amino acid substitution in an encoded HRAS polypeptide;
(e) a mutation in a MAP2K1 gene, optionally wherein the mutation results in a E102_I103del mutation and/or a K57T amino acid substitution in an encoded MAP2K1 polypeptide; and/or
(f) a mutation in an NRAS gene, optionally wherein the mutation results in a Q61K amino acid substitution in an encoded NRAS polypeptide.

Exemplary Clause 278: The method of clause 277, further comprising acquiring knowledge of or detecting, in a sample from the individual, the presence of:
a mutation in a BRAF gene resulting in an V600E amino acid substitution in an encoded BRAF polypeptide;
a mutation in an EGFR gene resulting in a S492R and/or V441G amino acid substitution in an encoded EGFR polypeptide;
a wild type KRAS gene;
a mutation in an HRAS gene resulting in an Q61L amino acid substitution in an encoded HRAS polypeptide;
a mutation in a MAP2K1 gene resulting in a E102_I103del mutation and/or a K57T amino acid substitution in an encoded MAP2K1 polypeptide; and
a mutation in an NRAS gene resulting in an Q61K amino acid substitution in an encoded NRAS polypeptide,
wherein the RAF1 fusion nucleic acid molecule is a RAF1-SYN2 fusion nucleic acid molecule as listed in any of Tables 1 and 3-5.

Exemplary Clause 279: The method of clause 278, wherein the cancer is a colorectal cancer.

Exemplary Clause 280: The method of clause 278 or clause 279, wherein the cancer was previously treated with 5-FU; folinic acid, 5-FU, and irinotecan (FOLFIRI) in combination with bevacizumab; FOLFIRI in combination with cetuximab; folinic acid, 5-FU, and oxaliplatin (FOLFOX) in combination with bevacizumab; and/or pembrolizumab in combination with regorafenib.

Exemplary Clause 281: The method of clause 277, further comprising acquiring knowledge of or detecting, in a sample from the individual, the presence of:
a mutation in a KRAS gene resulting in a G12C and/or G13D amino acid substitution in an encoded KRAS polypeptide;
a mutation in a MAP2K1 gene resulting in a E102_I103del mutation in an encoded MAP2K1 polypeptide; and
a mutation in an NRAS gene resulting in an Q61K amino acid substitution in an encoded NRAS polypeptide,
wherein the RAF1 fusion nucleic acid molecule is a RAF1-TRAK1 fusion nucleic acid molecule as listed in Tables 2 or 6.

Exemplary Clause 282: The method of clause 281, wherein the cancer is a colorectal cancer.

Exemplary Clause 283: The method of clause 281 or clause 282, wherein the cancer was previously treated with adagrasib or adagrasib in combination with cetuximab.

Exemplary Clause 284: The method of any one of clauses 271-283, wherein the anti-cancer therapy is a RAF1-targeted therapy.

Exemplary Clause 285: The method of clause 284, wherein the RAF1-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for a RAF1-rearranged cancer, a RAF1-targeted therapy being tested in a clinical trial, a treatment for RAF1-rearranged cancer being tested in a clinical trial, or any combination thereof.

Exemplary Clause 286: The method of clause 284 or clause 285, wherein the RAF1-targeted therapy is a kinase inhibitor.

Exemplary Clause 287: The method of any one of clauses 284-286, wherein the RAF1-targeted therapy is a serine/threonine kinase inhibitor.

Exemplary Clause 288: The method of any one of clauses 284-287, wherein the RAF1-targeted therapy is a multi-kinase inhibitor or a RAF1-specific inhibitor.

Exemplary Clause 289: The method of any one of clauses 286-288, wherein the kinase inhibitor inhibits a kinase activity of a RAF1 polypeptide.

Exemplary Clause 290: The method of any one of clauses 284-289, wherein the RAF1-targeted therapy comprises one or more of Sorafenib (BAY49-9006), Binimetinib, Cobimetinib, Regorafenib, Trametinib, or RAF265.

Exemplary Clause 291: The method of any one of clauses 285-290, wherein the nucleic acid inhibits the expression of the RAF1 fusion nucleic acid molecule or fusion polypeptide encoded by the fusion nucleic acid molecule.

Exemplary Clause 292: The method of any one of clauses 285-291, wherein the nucleic acid is a double-stranded RNA (dsRNA), a small interfering RNA (siRNA), or a small hairpin RNA (shRNA).

Exemplary Clause 293: The method of any one of clauses 285-292, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

Exemplary Clause 294: The method of any one of clauses 1-72, wherein the fusion nucleic acid molecule is a RET fusion nucleic acid molecule as listed in any of Tables 1-6.

Exemplary Clause 295: The method of clause 294, wherein the RET fusion nucleic acid molecule encodes a RET fusion polypeptide.

Exemplary Clause 296: The method of clause 295, wherein the encoded RET fusion polypeptide comprises a RET kinase domain, or a fragment of a RET kinase domain having RET kinase activity.

Exemplary Clause 297: The method of clause 295 or clause 296, wherein the encoded RET fusion polypeptide has RET kinase activity, optionally wherein the RET kinase activity is constitutive.

Exemplary Clause 298: The method of any one of clauses 295-297, wherein the encoded RET fusion polypeptide is oncogenic.

Exemplary Clause 299: The method of any one of clauses 295-298, wherein the encoded RET fusion polypeptide promotes cancer cell survival, angiogenesis, cancer cell proliferation, and any combination thereof.

Exemplary Clause 300: The method of any one of clauses 294-299, further comprising acquiring knowledge of or detecting, in a sample from the individual, the presence of:
(a) a mutation in an EGFR gene; optionally wherein the mutation is a deletion of exon 19 of EGFR or a portion thereof, or a mutation resulting in a T790M amino acid substitution in an encoded EGFR polypeptide, or both;
(b) a mutation in a PIK3CA gene; optionally wherein the mutation results in an E542K amino acid substitution in an encoded PIK3CA polypeptide;
(c) a mutation in a KRAS gene; optionally wherein the mutation results in a G12C amino acid substitution in an encoded KRAS polypeptide;
(d) a mutation in an ESR1 gene; optionally wherein the mutation results in an E380Q amino acid substitution in an encoded ESR1 polypeptide;
(e) a mutation in a PTEN gene; optionally wherein the mutation results in a S59* and/or M134I amino acid substitution in an encoded PTEN polypeptide; or any combination of (a)-(e).

Exemplary Clause 301: The method of any one of clauses 294-299, further comprising acquiring knowledge of or detecting, in a sample from the individual, the presence of a deletion of exon 19 of EGFR or a portion thereof; wherein the RET fusion nucleic acid molecule is a RET-ERC1 fusion nucleic acid molecule as listed in Tables 2 or 6.

Exemplary Clause 302: The method of any one of clauses 294-299, further comprising acquiring knowledge of or detecting, in a sample from the individual, the presence of a deletion of exon 19 of EGFR or a portion thereof, and an EGFR gene mutation resulting in a T790M amino acid substitution in an encoded EGFR polypeptide; wherein the RET fusion nucleic acid molecule is a RET-NCOA4 fusion nucleic acid molecule as listed in Tables 2 or 6.

Exemplary Clause 303: The method of clause 302, wherein the individual was previously treated with osimertinib.

Exemplary Clause 304: The method of any one of clauses 294-303, wherein the fusion nucleic acid molecule, and/or the fusion polypeptide encoded by the fusion nucleic acid molecule, confers resistance of the cancer to an EGFR-targeted anti-cancer therapy, optionally wherein the EGFR-targeted anti-cancer therapy is a first-, second-, or third-generation EGFR tyrosine kinase inhibitor.

Exemplary Clause 305: The method of any one of clauses 294-303, wherein the fusion nucleic acid molecule, and/or the fusion polypeptide encoded by the fusion nucleic acid molecule, confers resistance of the cancer to an EGFR-targeted anti-cancer therapy, optionally wherein the EGFR-targeted anti-cancer therapy is cetuximab, panitumumab, lapatinib, gefitinib, vandetanib, dacomitinib, icotinib, osimertinib (AZD9291), afatanib, olmutinib, EGF816 (nazartinib), avitinib (AC00010), rociletinib (CO-1686), BMS-690514, YH5448, PF-06747775, ASP8273, PF299804, AP26113, or erlotinib.

Exemplary Clause 306: The method of any one of clauses 294-299, further comprising acquiring knowledge of or detecting, in a sample from the individual, the presence of: a PIK3CA gene mutation resulting in an E542K amino acid substitution in an encoded PIK3CA polypeptide, an ESR1 gene mutation resulting in a E380Q amino acid substitution in an encoded ESR1 polypeptide, a KRAS gene mutation resulting in a G12C amino acid substitution in an encoded KRAS polypeptide, and a PTEN gene mutation resulting in a S59* and/or M134I amino acid substitution in an encoded PTEN polypeptide; wherein the RET fusion nucleic acid molecule is a RET-BAIAP2L1 fusion nucleic acid molecule as listed in any of Tables 1 and 3-5.

Exemplary Clause 307: The method of clause 306, wherein the cancer is a breast cancer.

Exemplary Clause 308: The method of any one of clauses 294-307, wherein the fusion nucleic acid molecule, and/or the fusion polypeptide encoded by the fusion nucleic acid molecule, confers resistance of the cancer to a PI3K-targeted therapy.

Exemplary Clause 309: The method of any one of clauses 294-299, further comprising acquiring knowledge of or detecting, in a sample from the individual, the presence of an EGFR gene mutation resulting in a T790M and/or L858R amino acid substitution in an encoded EGFR polypeptide; wherein the RET fusion nucleic acid molecule is a RET-CCDC6 fusion nucleic acid molecule as listed in Tables 2 or 6.

Exemplary Clause 310: The method of any one of clauses 294-309, wherein the anti-cancer therapy is a RET-targeted therapy.

Exemplary Clause 311: The method of clause 310, wherein the RET-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for a RET-rearranged cancer, a RET-targeted therapy being tested in a clinical trial, a treatment for RET-rearranged cancer being tested in a clinical trial, or any combination thereof.

Exemplary Clause 312: The method of clause 310 or clause 311, wherein the RET-targeted therapy is a kinase inhibitor.

Exemplary Clause 313: The method of any one of clauses 310-312, wherein the RET-targeted therapy is a tyrosine kinase inhibitor.

Exemplary Clause 314: The method of any one of clauses 310-313, wherein the RET-targeted therapy is a multi-kinase inhibitor or a RET-specific inhibitor.

Exemplary Clause 315: The method of any one of clauses 312-314, wherein the kinase inhibitor inhibits a kinase activity of a RET polypeptide.

Exemplary Clause 316: The method of any one of clauses 310-315, wherein the RET-targeted therapy comprises one or more of Selpercatinib, Pralsetinib, Alectinib, Cabozantinib, Lenvatinib, Ponatinib, Regorafenib, Sorafenib, Sunitinib, or Vandetanib.

Exemplary Clause 317: The method of any one of clauses 311-316, wherein the nucleic acid inhibits the expression of the RET fusion nucleic acid molecule or fusion polypeptide encoded by the fusion nucleic acid molecule.

Exemplary Clause 318: The method of any one of clauses 311-317, wherein the nucleic acid is a double-stranded RNA (dsRNA), a small interfering RNA (siRNA), or a small hairpin RNA (shRNA).

Exemplary Clause 319: The method of any one of clauses 311-318, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

Exemplary Clause 320: The method of any one of clauses 1-72, wherein the fusion nucleic acid molecule is a ROS1 fusion nucleic acid molecule as listed in any of Tables 1-6.

Exemplary Clause 321: The method of clause 320, wherein the ROS1 fusion nucleic acid molecule encodes a ROS1 fusion polypeptide.

Exemplary Clause 322: The method of clause 321, wherein the encoded ROS1 fusion polypeptide comprises a ROS1 kinase domain, or a fragment of a ROS1 kinase domain having ROS1 kinase activity.

Exemplary Clause 323: The method of clause 321 or clause 322, wherein the encoded ROS1 fusion polypeptide has ROS1 kinase activity, optionally wherein the ROS1 kinase activity is constitutive.

Exemplary Clause 324: The method of any one of clauses 321-323, wherein the encoded ROS1 fusion polypeptide is oncogenic.

Exemplary Clause 325: The method of any one of clauses 321-324, wherein the encoded ROS1 fusion polypeptide promotes cancer cell survival, angiogenesis, cancer cell proliferation, and any combination thereof.

Exemplary Clause 326: The method of any one of clauses 320-325, further comprising acquiring knowledge of or detecting, in a sample from the individual, the presence of a PIK3CA gene mutation; optionally wherein the mutation results in an E545K amino acid substitution in an encoded PIK3CA polypeptide.

Exemplary Clause 327: The method of clause 326, wherein the ROS1 fusion nucleic acid molecule is a ROS1-GOPC fusion nucleic acid molecule listed Tables 2 or 6, and wherein the sample comprises a PIK3CA gene mutation resulting in an E545K amino acid substitution in an encoded PIK3CA polypeptide.

Exemplary Clause 328: The method of any one of clauses 320-327, wherein the fusion nucleic acid molecule, and/or the fusion polypeptide encoded by the fusion nucleic acid molecule, confers resistance of the cancer to a PI3K-targeted therapy.

Exemplary Clause 329: The method of any one of clauses 320-328, wherein the anti-cancer therapy is a ROS1-targeted therapy.

Exemplary Clause 330: The method of clause 329, wherein the ROS1-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for a ROS1-rearranged cancer, a ROS1-targeted therapy being tested in a clinical trial, a treatment for ROS1-rearranged cancer being tested in a clinical trial, or any combination thereof.

Exemplary Clause 331: The method of clause 329 or clause 330, wherein the ROS1-targeted therapy is a kinase inhibitor.

Exemplary Clause 332: The method of any one of clauses 329-331, wherein the ROS1-targeted therapy is a tyrosine kinase inhibitor.

Exemplary Clause 333: The method of any one of clauses 329-332, wherein the ROS1-targeted therapy is a multi-kinase inhibitor or a ROS1-specific inhibitor.

Exemplary Clause 334: The method of any one of clauses 331-333, wherein the kinase inhibitor inhibits a kinase activity of a ROS1 polypeptide.

Exemplary Clause 335: The method of any one of clauses 329-334, wherein the ROS1-targeted therapy comprises one or more of crizotinib, lorlatinib, TQ-B3139, repotrectinib (TPX-0005), brigatinib, cabozantinib, ceritinib, or entrectinib.

Exemplary Clause 336: The method of any one of clauses 330-335, wherein the nucleic acid inhibits the expression of the ROS1 fusion nucleic acid molecule or fusion polypeptide encoded by the fusion nucleic acid molecule.

Exemplary Clause 337: The method of any one of clauses 330-336, wherein the nucleic acid is a double-stranded RNA (dsRNA), a small interfering RNA (siRNA), or a small hairpin RNA (shRNA).

Exemplary Clause 338: The method of any one of clauses 330-337, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

Exemplary Clause 339: The method of any one of clauses 1-72, wherein the fusion nucleic acid molecule is an NTRK1 fusion nucleic acid molecule as listed in any of Tables 2 and 6.

Exemplary Clause 340: The method of clause 339, wherein the NTRK1 fusion nucleic acid molecule encodes an NTRK1 fusion polypeptide.

Exemplary Clause 341: The method of clause 340, wherein the encoded NTRK1 fusion polypeptide comprises an NTRK1 kinase domain, or a fragment of an NTRK1 kinase domain having NTRK1 kinase activity.

Exemplary Clause 342: The method of clause 340 or clause 341, wherein the encoded NTRK1 fusion polypeptide has NTRK1 kinase activity, optionally wherein the NTRK1 kinase activity is constitutive.

Exemplary Clause 343: The method of any one of clauses 340-342, wherein the encoded NTRK1 fusion polypeptide is oncogenic.

Exemplary Clause 344: The method of any one of clauses 340-343, wherein the encoded NTRK1 fusion polypeptide promotes cancer cell survival, angiogenesis, cancer cell proliferation, and any combination thereof.

Exemplary Clause 345: The method of any one of clauses 339-344, wherein the anti-cancer therapy is an NTRK1-targeted therapy.

Exemplary Clause 346: The method of clause 345, wherein the NTRK1-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for an NTRK1-rearranged cancer, an NTRK1-targeted therapy being tested in a clinical trial, a treatment for NTRK1-rearranged cancer being tested in a clinical trial, or any combination thereof.

Exemplary Clause 347: The method of clause 345 or clause 346, wherein the NTRK1-targeted therapy is a kinase inhibitor.

Exemplary Clause 348: The method of any one of clauses 345-347, wherein the NTRK1-targeted therapy is a tyrosine kinase inhibitor.

Exemplary Clause 349: The method of any one of clauses 345-348, wherein the NTRK1-targeted therapy is a multi-kinase inhibitor or an NTRK1-specific inhibitor.

Exemplary Clause 350: The method of any one of clauses 347-349, wherein the kinase inhibitor inhibits a kinase activity of an NTRK1 polypeptide.

Exemplary Clause 351: The method of any one of clauses 345-350, wherein the NTRK1-targeted therapy comprises one or more of altiratinib (DCC-2701), AG 879 (Tyrphostin AG 879), an anti-TrK antibody, ARRY 954, AR523, AZ-23, AZ623, a benzotriazole, CEP-2563, danusertib (PHA-739358), entrectinib, DS-6051, GNF 5837, GW 441756, indenopyrrolocarboazole 12a, isothiazole 5n, larotrectinib, lestaurtinib (CEP-701), selitrectinib (LOXO-195), a macrocyclic compound, ONO-5390556, oxindole 3, pegcantratinib (SNA-120), PHA-848125, PLX7486, a pyrazole derivative, a pyrazolo[1; 5a]pyrimidine, a pyridocarbazole, a pyridoquinazolinyl, a pyridotriazole, a pyrrolidinyl thiourea, a pyrrolidinyl urea, a pyrrolo[2; 3-d]pyrimidine, a quinazolinyl, repotrectinib (TPX-0005), Ro 08-2750, a substituted pyrazolo[1; 5a]pyrimidine, sitravatinib (MGCD516), SNA-125, tavilermide, thiazole 20h, F17752, cabozantinib (XL184), merestinib (LY2801653), belizatinib (TSR-011), dovitinib, ONO-7579, or VMD-928.

Exemplary Clause 352: The method of any one of clauses 346-351, wherein the nucleic acid inhibits the expression of the NTRK1 fusion nucleic acid molecule or fusion polypeptide encoded by the fusion nucleic acid molecule.

Exemplary Clause 353: The method of any one of clauses 346-352, wherein the nucleic acid is a double-stranded RNA (dsRNA), a small interfering RNA (siRNA), or a small hairpin RNA (shRNA).

Exemplary Clause 354: The method of any one of clauses 346-353, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

Exemplary Clause 355: The method of any one of clauses 1-9, 18, 37-38, and 40-354, wherein the treatment or the one or more treatment options further comprise an additional anti-cancer therapy.

Exemplary Clause 356: The method of clause 355, wherein the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof.

Exemplary Clause 357: The method of clause 356, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

Exemplary Clause 358: The method of clause 356, wherein the nucleic acid comprises a double-stranded RNA (dsRNA), a small interfering RNA (siRNA), or a small hairpin RNA (shRNA).

Exemplary Clause 359: The method of any one of clauses 1-358, further comprising obtaining the sample from the individual.

Exemplary Clause 360: The method of any one of clauses 1-359, wherein the sample is obtained from the cancer.

Exemplary Clause 361: The method of any one of clauses 1-360, wherein the sample comprises a tissue biopsy sample, a liquid biopsy sample, or a normal control.

Exemplary Clause 362: The method of clause 361, wherein the sample is from a tumor biopsy, tumor specimen, or circulating tumor cell.

Exemplary Clause 363: The method of any one of clauses 1-361, wherein the sample is a liquid biopsy sample and comprises blood, plasma, cerebrospinal fluid, sputum, stool, urine, or saliva.

Exemplary Clause 364: The method of any one of clauses 1-363, wherein the sample comprises cells and/or nucleic acids from the cancer.

Exemplary Clause 365: The method of clause 364, wherein the sample comprises mRNA, DNA, circulating tumor DNA (ctDNA), cell-free DNA, or cell-free RNA from the cancer.

Exemplary Clause 366: The method of clause 363, wherein the sample is a liquid biopsy sample and comprises circulating tumor cells (CTCs).

Exemplary Clause 367: The method of clause 363, wherein the sample is a liquid biopsy sample and comprises cell-free DNA (cfDNA), circulating tumor DNA (ctDNA), or any combination thereof.

Exemplary Clause 368: The method of any one of clauses 1-367, comprising acquiring knowledge of or detecting the fusion nucleic acid molecule or the fusion polypeptide encoded by the fusion nucleic acid molecule in a tissue biopsy sample, in a liquid biopsy sample, or in both a tissue biopsy sample and a liquid biopsy sample, from the individual.

Exemplary Clause 369: The method of any one of clauses 4-10 and 50-368, wherein the acquiring knowledge comprises detecting the fusion nucleic acid molecule, or the polypeptide encoded by the fusion nucleic acid molecule, in the sample.

Exemplary Clause 370: The method of any one of clauses 1-3, 11-41, and 49-369, wherein the detecting comprises detecting a fragment of the fusion nucleic acid molecule comprising a breakpoint or fusion junction.

Exemplary Clause 371: The method of any one of clauses 1-3, 11-41, and 49-370, wherein the fusion nucleic acid molecule is detected in the sample by one or more of: a nucleic acid hybridization assay, an amplification-based assay, a polymerase chain reaction-restriction fragment length polymorphism (PCR-RFLP) assay, real-time PCR, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), mass-spectrometric genotyping, or sequencing.

Exemplary Clause 372: The method of clause 371, wherein the sequencing comprises a massively parallel sequencing (MPS) technique, whole genome sequencing (WGS), whole exome sequencing, targeted sequencing, direct sequencing, or a Sanger sequencing technique; and optionally wherein the massively parallel sequencing (MPS) technique comprises next-generation sequencing (NGS).

Exemplary Clause 373: The method of any one of clauses 1-3, 11-41, and 49-369, wherein detecting the fusion polypeptide encoded by the fusion nucleic acid molecule comprises detecting a portion of the fusion polypeptide that is encoded by a fragment of the fusion nucleic acid molecule that comprises a breakpoint or a fusion junction.

Exemplary Clause 374: The method of any one of clauses 1-3, 11-41, 49-369, and 373, wherein the fusion polypeptide is detected in the sample by one or more of: immunoblotting, enzyme linked immunosorbent assay (ELISA), immunohistochemistry, or mass spectrometry.

Exemplary Clause 375: The method of any one of clauses 1-3, 11-18, and 42-372, further comprising selectively enriching for one or more nucleic acids in the sample comprising nucleotide sequences corresponding to the fusion nucleic acid molecule; wherein the selectively enriching produces an enriched sample.

Exemplary Clause 376: The method of clause 375, wherein the selectively enriching comprises: (a) combining one or more bait molecules with the sample, thereby hybridizing the one or more bait molecules to one or more nucleic acids in the sample comprising nucleotide sequences corresponding to the fusion nucleic acid molecule and producing nucleic acid hybrids; and (b) isolating the nucleic acid hybrids to produce the enriched sample.

Exemplary Clause 377: The method of any one of clauses 22, 28-41, and 376, wherein the one or more bait molecules comprise a capture nucleic acid molecule configured to hybridize to a nucleotide sequence corresponding to the fusion nucleic acid molecule.

Exemplary Clause 378: The method of clause 377, wherein the capture nucleic acid molecule comprises between about 10 and about 30 nucleotides, between about 50 and about 1000 nucleotides, between about 100 and about 500 nucleotides, between about 100 and about 300 nucleotides, or between about 100 and about 200 nucleotides.

Exemplary Clause 379: The method of any one of clauses 22, 28-41, and 376-378, wherein the one or more bait molecules are conjugated to an affinity reagent or to a detection reagent.

Exemplary Clause 380: The method of clause 379, wherein the affinity reagent is an antibody, an antibody fragment, or biotin, or wherein the detection reagent is a fluorescent marker.

Exemplary Clause 381: The method of any one of clauses 377-380, wherein the capture nucleic acid molecule comprises a DNA, RNA, or mixed DNA/RNA molecule.

Exemplary Clause 382: The method of any one of clauses 23-27, 30-41, and 50-375, wherein the selectively enriching comprises amplifying the one or more nucleic acids comprising nucleotide sequences corresponding to the fusion nucleic acid molecule using a polymerase chain reaction (PCR) to produce an enriched sample.

Exemplary Clause 383: The method of any one of clauses 375-382, further comprising sequencing the enriched sample.

Exemplary Clause 384: The method of any one of clauses 1-383, wherein the individual is a human.

Exemplary Clause 385: A kit comprising a probe or bait for detecting:
(i) an ALK, BRAF, EGFR, ERBB2, FGFR1, FGFR2, FGFR3, MET, RAF1, RET, or ROS1 fusion nucleic acid molecule listed in any of Tables 1 and 3-5, or a fragment thereof comprising a breakpoint or fusion junction; or
(ii) an ALK, BRAF, ERBB2, FGFR1, FGFR2, FGFR3, MET, NTRK1, RAF1, RET, or ROS1 fusion nucleic acid molecule listed in Tables 2 or 6, or a fragment thereof comprising a breakpoint or fusion junction, in a sample from an individual having a cancer corresponding to the ALK, BRAF, ERBB2, FGFR1, FGFR2, FGFR3, MET, NTRK1, RAF1, RET, or ROS1 fusion nucleic acid molecule as listed in Tables 2 or 6.

Exemplary Clause 386: A nucleic acid molecule comprising an ALK, BRAF, EGFR, ERBB2, FGFR1, FGFR2, FGFR3, MET, RAF1, RET, or ROS1 fusion nucleic acid molecule listed in any of Tables 1 and 3-5, or a fragment thereof comprising a breakpoint or fusion junction.

Exemplary Clause 387: A vector comprising the nucleic acid molecule of clause 386.

Exemplary Clause 388: A host cell comprising the vector of clause 387.

Exemplary Clause 389: An antibody or antibody fragment that specifically binds to a fusion polypeptide, or to a portion thereof, encoded by an ALK, BRAF, EGFR, ERBB2, FGFR1, FGFR2, FGFR3, MET, RAF1, RET, or ROS1 fusion nucleic acid molecule listed in any of Tables 1 and 3-5, or a fragment thereof comprising a breakpoint or fusion junction.

Exemplary Clause 390: A kit comprising an antibody or antibody fragment for detecting:
(i) a fusion polypeptide, or a portion thereof, encoded by an ALK, BRAF, EGFR, ERBB2, FGFR1, FGFR2, FGFR3, MET, RAF1, RET, or ROS1 fusion nucleic acid molecule listed in any of Tables 1 and 3-5, or a fragment thereof comprising a breakpoint or fusion junction; or
(ii) a fusion polypeptide, or a portion thereof, encoded by an ALK, BRAF, ERBB2, FGFR1, FGFR2, FGFR3, MET, NTRK1, RAF1, RET, or ROS1 fusion nucleic acid molecule listed in Tables 2 or 6, or a fragment thereof comprising a breakpoint or fusion junction, in a sample from an individual having a cancer corresponding to the ALK, BRAF, ERBB2, FGFR1, FGFR2, FGFR3, MET, NTRK1, RAF1, RET, or ROS1 fusion nucleic acid molecule as listed in Tables 2 or 6.

Exemplary Clause 391: In vitro use of one or more oligonucleotides for detecting:
(i) an ALK, BRAF, EGFR, ERBB2, FGFR1, FGFR2, FGFR3, MET, RAF1, RET, or ROS1 fusion nucleic acid molecule listed in any of Tables 1 and 3-5, or a fragment thereof comprising a breakpoint or fusion junction; or
(ii) an ALK, BRAF, ERBB2, FGFR1, FGFR2, FGFR3, MET, NTRK1, RAF1, RET, or ROS1 fusion nucleic acid molecule listed in Tables 2 or 6, or a fragment thereof comprising a breakpoint or fusion junction, in a sample from an individual having a cancer corresponding to the ALK, BRAF, ERBB2, FGFR1, FGFR2, FGFR3, MET, NTRK1, RAF1, RET, or ROS1 fusion nucleic acid molecule as listed in Tables 2 or 6.

Exemplary Clause 392: A kit comprising one or more oligonucleotides for detecting:
(i) an ALK, BRAF, EGFR, ERBB2, FGFR1, FGFR2, FGFR3, MET, RAF1, RET, or ROS1 fusion nucleic acid molecule listed in any of Tables 1 and 3-5, or a fragment thereof comprising a breakpoint or fusion junction; or
(ii) an ALK, BRAF, ERBB2, FGFR1, FGFR2, FGFR3, MET, NTRK1, RAF1, RET, or ROS1 fusion nucleic acid molecule listed in Tables 2 or 6, or a fragment thereof comprising a breakpoint or fusion junction, in a sample from an individual having a cancer corresponding to the ALK, BRAF, ERBB2, FGFR1, FGFR2, FGFR3, MET, NTRK1, RAF1, RET, or ROS1 fusion nucleic acid molecule as listed in Tables 2 or 6.

Exemplary Clause 393: A system, comprising:
a memory configured to store one or more program instructions; and
one or more processors configured to execute the one or more program instructions, the one or more program instructions when executed by the one or more processors are configured to:
(a) obtain a plurality of sequence reads of one or more nucleic acid molecules, wherein the one or more nucleic acid molecules are derived from a sample obtained from an individual;
(b) analyze the plurality of sequence reads for the presence of a fusion nucleic acid molecule, wherein the fusion nucleic acid molecule is an ALK, BRAF, EGFR, ERBB2, FGFR1, FGFR2, FGFR3, MET, RAF1, RET, or ROS1 fusion nucleic acid molecule listed in Table 1; and
(c) detect, based on the analyzing, the fusion nucleic acid molecule in the sample.

Exemplary Clause 394: A non-transitory computer readable storage medium comprising one or more programs executable by one or more computer processors for performing a method, comprising:
(a) obtaining, using the one or more processors, a plurality of sequence reads of one or more nucleic acid molecules, wherein the one or more nucleic acid molecules are derived from a sample obtained from an individual;
(b) analyzing, using the one or more processors, the plurality of sequence reads for the presence of a fusion nucleic acid molecule, wherein the fusion nucleic acid molecule is an ALK, BRAF, EGFR, ERBB2, FGFR1, FGFR2, FGFR3, MET, RAF1, RET, or ROS1 fusion nucleic acid molecule listed in Table 1; and
(c) detecting, using the one or more processors and based on the analyzing, the fusion nucleic acid molecule in the sample.

Exemplary Clause 395: The system of clause 393, or the non-transitory computer readable storage medium of clause 394, wherein the sample is from an individual having a cancer.

Exemplary Clause 396: The system of clause 393 or clause 395, or the non-transitory computer readable storage medium of clause 394 or clause 395, wherein the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, comprising or resulting from a Breakpoint 1 and/or a Breakpoint 2 corresponding to the ALK, BRAF, EGFR, ERBB2, FGFR1, FGFR2, FGFR3, MET, RAF1, RET, or ROS1 fusion nucleic acid molecule as listed in Table 3.

Exemplary Clause 397: The system or the non-transitory computer readable storage medium of clause 395 or clause 396, wherein the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, and the cancer is a carcinoma, a sarcoma, a lymphoma, a leukemia, a myeloma, a germ cell cancer, or a blastoma.

Exemplary Clause 398: The system or the non-transitory computer readable storage medium of any one of clauses 395-397, wherein the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, and the cancer is a solid tumor.

Exemplary Clause 399: The system or the non-transitory computer readable storage medium of any one of clauses 395-397, wherein the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, and the cancer is a hematologic malignancy.

Exemplary Clause 400: The system or the non-transitory computer readable storage medium of any one of clauses 395-397, wherein the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, and the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

Exemplary Clause 401: The system or the non-transitory computer readable storage medium of any one of clauses 395-397, wherein the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, and the cancer is appendix adenocarcinoma, bladder adenocarcinoma, bladder urothelial (transitional cell) carcinoma, breast cancer not otherwise specified NOS, breast carcinoma NOS, breast invasive ductal carcinoma (IDC), breast invasive lobular carcinoma (ILC), cervix squamous cell carcinoma (SCC), colon adenocarcinoma (CRC), esophagus adenocarcinoma, esophagus carcinoma NOS, esophagus squamous cell carcinoma (SCC), eye intraocular melanoma, gallbladder adenocarcinoma, gastroesophageal junction adenocarcinoma, intra-hepatic cholangiocarcinoma, kidney cancer NOS, liver hepatocellular carcinoma (HCC), lung cancer NOS, lung adenocarcinoma, lung large cell carcinoma, lung non-small cell lung carcinoma (NSCLC) NOS, lung small cell undifferentiated carcinoma, lung squamous cell carcinoma (SCC), ovary cancer NOS, pancreas cancer NOS, pancreas ductal adenocarcinoma, pancreatobiliary carcinoma, prostate cancer NOS, prostate acinar adenocarcinoma, prostate ductal adenocarcinoma, rectum adenocarcinoma (CRC), skin melanoma, small intestine adenocarcinoma, soft tissue sarcoma NOS, stomach adenocarcinoma NOS, unknown primary cancer NOS, unknown primary adenocarcinoma, unknown primary carcinoma (CUP) NOS, unknown primary neuroendocrine tumor, unknown primary squamous cell carcinoma (SCC), or uterus endometrial adenocarcinoma NOS.

Exemplary Clause 402: The system or the non-transitory computer readable storage medium of any one of clauses 395-397, wherein:
(a) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, and the cancer is the cancer corresponding to the ALK, BRAF, EGFR, ERBB2, FGFR1, FGFR2, FGFR3, MET, RAF1, RET, or ROS1 fusion nucleic acid molecule as listed in Table 4; or
(b) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, the cancer is the cancer corresponding to the ALK, BRAF, EGFR, ERBB2, FGFR1, FGFR2, FGFR3, MET, RAF1, RET, or ROS1 fusion nucleic acid molecule as listed in Table 5, and the fusion nucleic acid molecule comprises or results from a Breakpoint 1 and/or a Breakpoint 2 corresponding to the ALK, BRAF, EGFR, ERBB2, FGFR1, FGFR2, FGFR3, MET, RAF1, RET, or ROS1 fusion nucleic acid molecule as listed in Table 5.

Exemplary Clause 403: A system, comprising:
a memory configured to store one or more program instructions; and
one or more processors configured to execute the one or more program instructions, the one or more program instructions when executed by the one or more processors are configured to:
(a) obtain a plurality of sequence reads of one or more nucleic acid molecules, wherein the one or more nucleic acid molecules are derived from a sample obtained from an individual having a cancer;
(b) analyze the plurality of sequence reads for the presence of a fusion nucleic acid molecule, wherein the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2; and
(c) detect, based on the analyzing, the fusion nucleic acid molecule in the sample.

Exemplary Clause 404: A non-transitory computer readable storage medium comprising one or more programs executable by one or more computer processors for performing a method, comprising:
(a) obtaining, using the one or more processors, a plurality of sequence reads of one or more nucleic acid molecules, wherein the one or more nucleic acid molecules are derived from a sample obtained from an individual having a cancer;
(b) analyzing, using the one or more processors, the plurality of sequence reads for the presence of a fusion nucleic acid molecule, wherein the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2; and
(c) detecting, using the one or more processors and based on the analyzing, the fusion nucleic acid molecule in the sample.

Exemplary Clause 405: The system of clause 403, or the non-transitory computer readable storage medium of clause 404, wherein the fusion nucleic acid molecule comprises or results from a Breakpoint 1 and/or a Breakpoint 2 corresponding to the ALK, BRAF, ERBB2, FGFR1, FGFR2, FGFR3, MET, NTRK1, RAF1, RET, or ROS1 fusion nucleic acid molecule as listed in Table 6.

Exemplary Clause 406: The system of any one of clauses 393, 395-403, and 405, or the non-transitory computer readable storage medium of any one of clauses 394-402 and 404-405, wherein the plurality of sequence reads is obtained by sequencing; optionally wherein the sequencing comprises use of a massively parallel sequencing (MPS) technique, whole genome sequencing (WGS), whole exome sequencing, targeted sequencing, direct sequencing, or a Sanger sequencing technique; and optionally wherein the massively parallel sequencing technique comprises next generation sequencing (NGS).

Exemplary Clause 407: The system of any one of clauses 393, 395-403, and 405-406, wherein the one or more program instructions when executed by the one or more processors are further configured to generate, based at least in part on the detecting, a genomic profile for the sample.

Exemplary Clause 408: The non-transitory computer readable storage medium of any one of clauses 394-402 and 404-406, wherein the method further comprises generating, based at least in part on the detecting, a genomic profile for the sample.

Exemplary Clause 409: The system of clause 407, or the non-transitory computer readable storage medium of clause 408, wherein the individual is administered a treatment based at least in part on the genomic profile.

Exemplary Clause 410: The system of any one of clauses 407 and 409, or the non-transitory computer readable storage medium of any one of clauses 408-409, wherein the genomic profile further comprises results from a comprehensive genomic profiling (CGP) test, a gene expression profiling test, a cancer hotspot panel test, a DNA methylation test, a DNA fragmentation test, an RNA fragmentation test, or any combination thereof.

Exemplary Clause 411: The system of any one of clauses 407 and 409-410, or the non-transitory computer readable storage medium of any one of clauses 408-410, wherein the genomic profile further comprises results from a nucleic acid sequencing-based test.

Exemplary Clause 412: An anti-cancer therapy for use in a method of treating or delaying progression of cancer, wherein the method comprises administering the anti-cancer therapy to an individual, wherein:
(a) an ALK, BRAF, EGFR, ERBB2, FGFR1, FGFR2, FGFR3, MET, RAF1, RET, or ROS1 fusion nucleic acid molecule listed in Table 1 or a fragment thereof comprising a breakpoint or fusion junction, or a fusion polypeptide encoded by the fusion nucleic acid molecule, is detected in a sample obtained from the individual; or
(b) an ALK, BRAF, ERBB2, FGFR1, FGFR2, FGFR3, MET, NTRK1, RAF1, RET, or ROS1 fusion nucleic acid molecule listed in Table 2 or a fragment thereof comprising a breakpoint or fusion junction, or a fusion polypeptide encoded by the fusion nucleic acid molecule, is detected in a sample obtained from the individual, wherein the individual has a cancer corresponding to the ALK, BRAF, ERBB2, FGFR1, FGFR2, FGFR3, MET, NTRK1, RAF1, RET, or ROS1 fusion nucleic acid molecule as listed in Table 2.

Exemplary Clause 413: An anti-cancer therapy for use in the manufacture of a medicament for treating or delaying progression of cancer, wherein the medicament is to be administered to an individual, wherein:
(a) an ALK, BRAF, EGFR, ERBB2, FGFR1, FGFR2, FGFR3, MET, RAF1, RET, or ROS1 fusion nucleic acid molecule listed in Table 1 or a fragment thereof comprising a breakpoint or fusion junction, or a fusion polypeptide encoded by the fusion nucleic acid molecule, is detected in a sample obtained from the individual; or
(b) an ALK, BRAF, ERBB2, FGFR1, FGFR2, FGFR3, MET, NTRK1, RAF1, RET, or ROS1 fusion nucleic acid molecule listed in Table 2 or a fragment thereof comprising a breakpoint or fusion junction, or a fusion polypeptide encoded by the fusion nucleic acid molecule, is detected in a sample obtained from the individual, wherein the individual has a cancer corresponding to the ALK, BRAF, ERBB2, FGFR1, FGFR2, FGFR3, MET, NTRK1, RAF1, RET, or ROS1 fusion nucleic acid molecule as listed in Table 2.

The method steps of the invention(s) described herein are intended to include any suitable method of causing one or more other parties or entities to perform the steps, unless a different meaning is expressly provided or otherwise clear from the context. Such parties or entities need not be under the direction or control of any other party or entity, and need not be located within a particular jurisdiction. Thus, for example, a description or recitation of “adding a first number to a second number” includes causing one or more parties or entities to add the two numbers together. For example, if person X engages in an arm's length transaction with person Y to add the two numbers, and person Y indeed adds the two numbers, then both persons X and Y perform the step as recited: person Y by virtue of the fact that he actually added the numbers, and person X by virtue of the fact that he caused person Y to add the numbers. Furthermore, if person X is located within the United States and person Y is located outside the United States, then the method is performed in the United States by virtue of person X's participation in causing the step to be performed.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

The specification is considered to be sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes. To the extent that any reference incorporated by reference conflicts with the instant disclosure, the instant disclosure shall control.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Kinase Fusions Detected in Circulating Tumor DNA (ctDNA) from a Diverse Range of Cancer Types

Kinase fusions are an important class of targetable oncogenic driver variants, and can also mediate acquired resistance (AR) to targeted therapies. Kinase fusions are detectable using hybrid-capture DNA sequencing, but can be challenging to detect in circulating tumor DNA (ctDNA). In this Example, next-generation sequencing (NGS) of ctDNA was used to characterize the pan-cancer landscape of kinase fusions.

### Methods

Hybrid-capture based NGS was performed in formalin-fixed paraffin-embedded (FFPE) tumor tissue or blood/plasma samples (i.e., tissue biopsy or liquid biopsy, respectively) prospectively collected from 405,847 patients in the course of clinical care. For tumor tissue samples, DNA was extracted from FFPE specimens and NGS was performed by hybridization-capture, adaptor ligation-based libraries to high, uniform coverage (>500×) for all coding exons of up to 324 cancer-related genes plus selected introns (see, e.g., Frampton et al. (2013) Nat Biotechnol, 31:1023-1031). NGS of ctDNA was performed on ≥20 ng of ctDNA extracted from blood plasma to create adapted sequencing libraries before hybrid capture and sample-multiplexed sequencing to a median unique exon coverage depth of >6,000× for up to 324 genes (see, e.g., Clark et al. (2018) J Mol Diagn 20:686-702; Woodhouse et al. (2020) PLoS One 15:e0237802). Base substitutions, short insertions and deletions (indels), copy number changes, and rearrangements were assessed. All exons plus selected introns of 16 kinases involved in oncogenic fusions (ALK, BRAF, EGFR, ERBB2, FGFR1/2/3, MET, NTRK1/2/3, PDGFRA/B, RAF1, RET, and ROS1) were sequenced to capture fusions.

The ctDNA fraction in plasma cell-free DNA was estimated using two complementary methods. When ctDNA fraction was sufficient, a tumor fraction (TF) estimate was calculated based on a measure of tumor aneuploidy that incorporated observed deviations in coverage across the genome for a given sample. Calculated values for this metric were calibrated against a training set based on samples with well-defined TFs to generate an estimate of TF. When ctDNA content was lower, the maximum somatic allele frequency (MSAF) was determined by calculating the allele fraction for all known somatic, likely somatic, and variant of unknown significance (VUS) base substitutions, excluding certain common and rare germline variants. The estimated ctDNA fraction was based on the TF estimate when available, and was generated from MSAF when lack of tumor aneuploidy limited the ability to return an informative estimate of TF.

A Fisher's exact test was used to assess significance of categorical relationships, and the Benjamin-Hochberg procedure was used to control for false discovery rate. Differences between continuous variables were assessed by the Mann-Whitney U test.

Liquid biopsy results with no evidence of ctDNA variants were excluded from the studies described herein.

Detected kinase fusions were considered in the studies described herein if the fusion product included the full kinase domain of the target oncogene (ALK, BRAF, EGFR, ERBB2, FGFR1/2/3, MET, NTRK1/2/3, PDGFRA/B, RAF1, RET, and ROS1), and the predicted chimeric protein included both an N-terminus and a C-terminus.

### Results

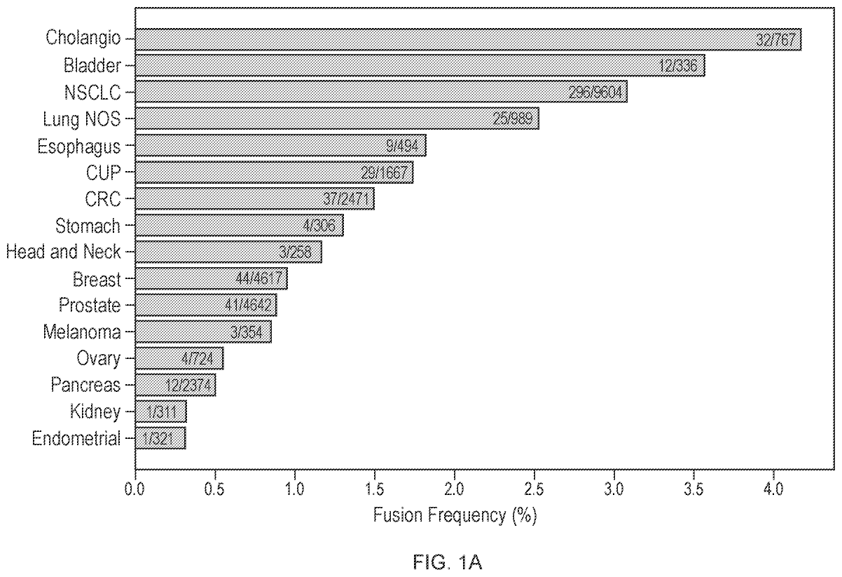

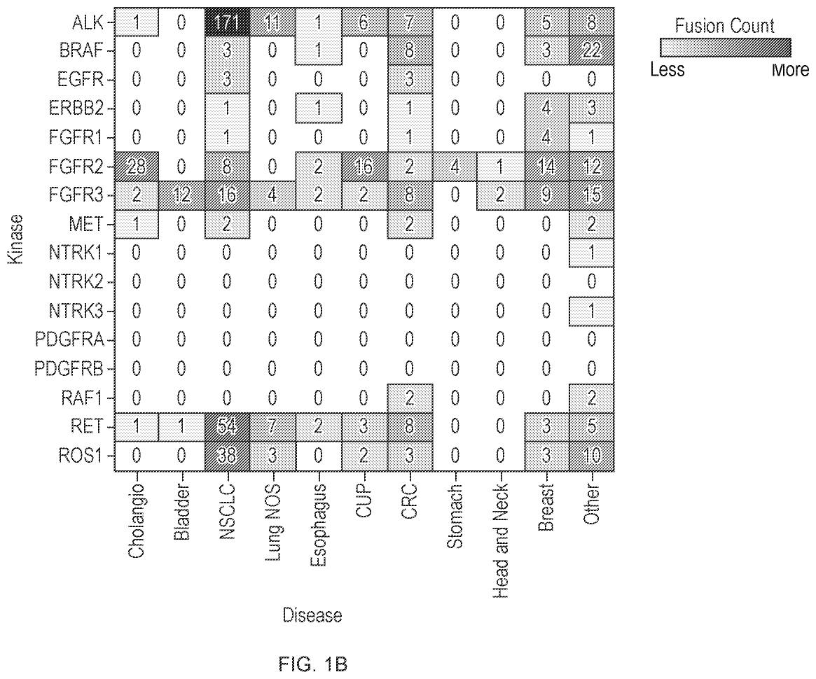

Of 36,916 plasma samples, 32,492 (88%) had detectable ctDNA. Overall, 571 (1.8%) of those samples featured kinase fusions detected in ctDNA (131 using a blood-based assay for circulating tumor DNA [BBACTD], 203 using a blood-based liquid biopsy assay #1 [BBLB1], and 237 using a blood-based liquid biopsy assay #2 [BBLB2]; Tables 7 and 10). Cholangiocarcinoma samples showed the highest frequency of kinase fusions (4.2%), followed by bladder cancer (3.6%), and non-small cell lung cancer (NSCLC, 3.1%) (FIG. 1A and Table 8). Among 297 fusions detected in 296 NSCLC cases, the most frequently rearranged kinases were ALK (171, 58%), RET (54, 18%) and ROS1 (38, 13%) (FIG. 1B). In cholangiocarcinoma, FGFR2 (85%, 28/33) was the most commonly rearranged kinase, while in bladder cancer, FGFR3 was the most commonly rearranged kinase (92%, 12/13) (FIG. 1B).

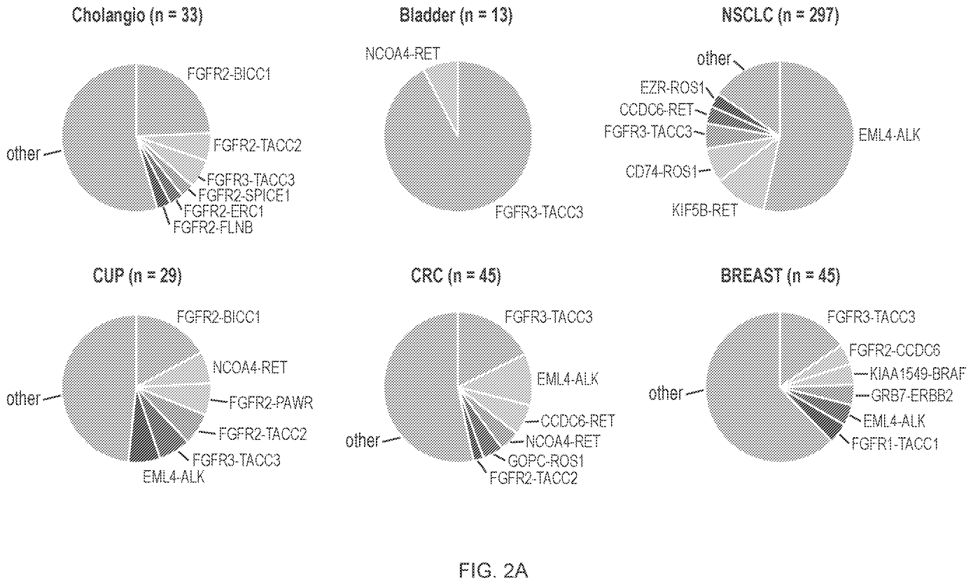

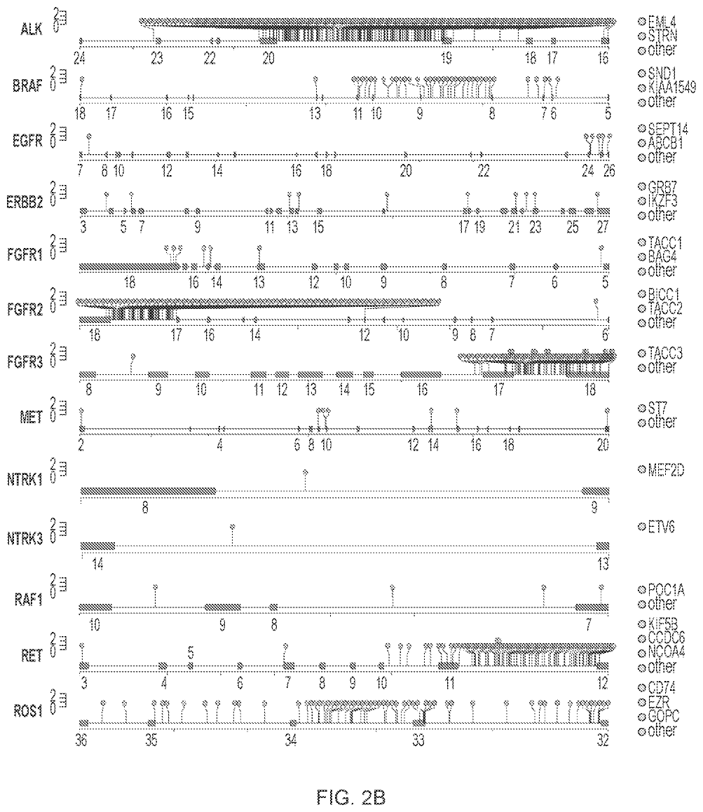

In NSCLC, kinase fusions involved diverse partners, including most commonly EML4-ALK (n=159), KIFSB-RET (n=34), and CD74-ROS1 (n=22), while in bladder cancer, FGFR3 exclusively partnered with TACC3 (FIG. 2A). In cholangiocarcinoma, FGFR2 was most commonly fused to BICC1 (n=8) (FIG. 2A). ALK fusions including EML4-ALK have been identified in multiple tumor types (see, e.g., Lipson et al. (2012) Nat Med 18:382-384; Perot et al. (2014) PLoS One 9:e87170; Debelenko et al. (2011) Mod Pathol 24:430-442; Lin et al. (2009) Mol Cancer Res 7:1466-1476). As shown in Table 9, 9.6% ( 20/209) of ALK fusion-positive cases were non-lung, including one cholangiocarcinoma case with an EML4-ALK fusion. Among 210 ALK fusions, 188 (90%) occurred with a breakpoint in intron 19. Breakpoints were also observed in exon 20 (4.8%), intron 18 (1.9%), and exon 19 (1.9%) (FIG. 2B).

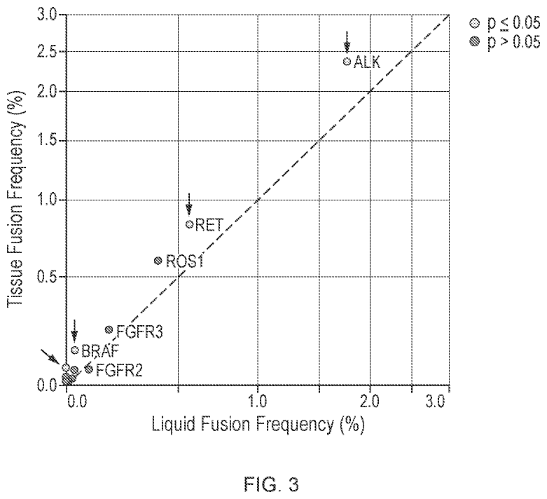

The frequency of 16 kinase fusions detected in ctDNA samples from patients with NSCLC (n=296) was compared to the frequency of the same fusions detected in tissue of patients with matched disease ontology. The overall prevalence of kinase fusions was slightly elevated in tissue samples compared with the prevalence in ctDNA samples for several kinases, including ALK (2.4% vs. 1.8%, p=0.003), RET (0.83% vs. 0.56%, p=0.02), BRAF (0.14% vs. 0.03%, p=0.02), and NTRK1 (0.07% vs. 0%, p=0.02) (FIG. 3). This may be partially attributable to differences in intron baiting between assay versions. The ctDNA assays used in this Example included intronic baiting for nine genes (ALK, RET, ROS1, FGFR2, FGFR3, EGFR, NTRK1, NTRK2, and PDGFRA) such that rearrangements with noncanonical breakpoints in other introns or involving genes without intron baiting may be underrepresented.

TABLE 7
Kinase fusions identified in ctDNA in various cancer types.

| Kinase Fusion | Cancer Type(s) |
|---|---|
| ALK - AGAP1 | Prostate acinar adenocarcinoma |
| ALK - ARHGEF7 | Esophagus carcinoma (not otherwise specified: NOS) |
| ALK - BRE | Lung non-small cell lung carcinoma (NSCLC) (NOS) |
| ALK - EML4 | Lung adenocarcinoma |
| | Lung adenosquamous carcinoma |
| | Lung non-small cell lung carcinoma (NSCLC) (NOS) |
| | Colon adenocarcinoma (colorectal cancer: CRC) |
| | Intra-hepatic cholangiocarcinoma |
| | Lung squamous cell carcinoma (SCC) |
| | Colorectal (CRC) (NOS) |
| | Lung (NOS) |
| | Unknown primary carcinoma (carcinoma of unknown |
| | primary: CUP) (NOS) |
| | Breast carcinoma (NOS) |
| | Breast (NOS) |
| ALK - EPS8 | Lung adenocarcinoma |
| ALK - GCC2 | Lung squamous cell carcinoma (SCC) |
| ALK - GPR113 | Prostate acinar adenocarcinoma |
| ALK - HDAC9 | Unknown primary carcinoma (CUP) (NOS) |
| ALK - HIP1 | Unknown primary carcinoma (CUP) (NOS) |
| | Lung non-small cell lung carcinoma (NSCLC) (NOS) |
| | Lung adenocarcinoma |
| ALK - KANK1 | Pancreas (NOS) |
| ALK - KIF5B | Lung (NOS) |
| ALK - KLC1 | Unknown primary adenocarcinoma |
| ALK - MIPOL1 | Colon adenocarcinoma (CRC) |
| ALK - PELI1 | Breast (NOS) |
| ALK - PPFIBP1 | Unknown primary (NOS) |
| ALK - SLC39A10 | Breast (NOS) |
| ALK - PLEKHA7 | Lung non-small cell lung carcinoma (NSCLC) (NOS) |
| ALK - STRN | Lung adenocarcinoma |
| | Lung non-small cell lung carcinoma (NSCLC) (NOS) |
| | Colon adenocarcinoma (CRC) |
| ALK - TFG | Breast carcinoma (NOS) |
| ALK - TPM3 | Soft tissue sarcoma undifferentiated |
| | Pancreas ductal adenocarcinoma |
| ALK - VKORCIL1 | Lung (NOS) |
| ALK - SORBS1 | Lung adenocarcinoma |
| ALK - SPINK5 | Prostate (NOS) |
| BRAF - AGK | Prostate (NOS) |
| BRAF - AKAP9 | Colon adenocarcinoma (CRC) |
| BRAF - ARMC10 | Esophagus adenocarcinoma |
| BRAF - CCDC88C | Lung non-small cell lung carcinoma (NSCLC) (NOS) |
| BRAF - COBLL1 | Pancreas ductal adenocarcinoma |
| BRAF - CREB3L2 | Prostate (NOS) |
| BRAF - CUL1 | Pancreas ductal adenocarcinoma |
| BRAF - CUX1 | Pancreas (NOS) |
| BRAF - DENND2A | Colon adenocarcinoma (CRC) |
| BRAF -DLC1 | Colon adenocarcinoma (CRC) |
| BRAF - GOLGA3 | Colon adenocarcinoma (CRC) |
| BRAF - JHDM1D | Prostate acinar adenocarcinoma |
| BRAF - KIAA1549 | Prostate acinar adenocarcinoma |
| | Breast carcinoma (NOS) |
| | Prostate (NOS) |
| | Skin melanoma |
| BRAF - MKRN1 | Rectum adenocarcinoma (CRC) |
| BRAF - MSI2 | Breast (NOS) |
| BRAF - NRF1 | Colon adenocarcinoma (CRC) |
| BRAF - SLC45A3 | Prostate acinar adenocarcinoma |
| | Prostate (NOS) |
| BRAF - SND1 | Prostate acinar adenocarcinoma |
| | Prostate (NOS) |
| | Prostate ductal adenocarcinoma |
| | Pancreas (NOS) |
| | Colon adenocarcinoma (CRC) |
| BRAF - TNS3 | Soft tissue sarcoma (NOS) |
| BRAF - TRIM24 | Lung adenocarcinoma |
| | Rectum adenocarcinoma (CRC) |
| BRAF - ZC3HAV1 | Colon adenocarcinoma (CRC) |
| BRAF - ZNF277 | Lung adenocarcinoma |
| BRAF - DOCK4 | Prostate acinar adenocarcinoma |
| EGFR - SEPT14 | Colon adenocarcinoma (CRC) |
| EGFR - ABCB1 | Lung non-small cell lung carcinoma (NSCLC) (NOS) |
| EGFR - PDE7A | Colon adenocarcinoma (CRC) |
| EGFR - EZH2 | Lung adenocarcinoma |
| EGFR - FLJ45974 | Colon adenocarcinoma (CRC) |
| EGFR - ZNF479 | Lung adenocarcinoma |
| ERBB2 - FBXL20 | Uterus endometrial adenocarcinoma (NOS) |
| ERBB2 - GRB7 | Breast invasive ductal carcinoma (IDC) |
| ERBB2 - IKZF3 | Breast (NOS) |
| ERBB2 - MSI2 | Gastroesophageal junction adenocarcinoma |
| ERBB2 - RANBP10 | Pancreas ductal adenocarcinoma |
| ERBB2 -SEC14L1 | Lung non-small cell lung carcinoma (NSCLC) (NOS) |
| ERBB2 - WIPF2 | Ovary (NOS) |
| ERBB2 - GRB7 | Breast (NOS) |
| ERBB2 - PPP1R1B | Colon adenocarcinoma (CRC) |
| ERBB2 - PRKCA | Breast carcinoma (NOS) |
| FGFR1 - ADAM32 | Breast (NOS) |
| FGFR1 -ADAM18 | Cervix squamous cell carcinoma (SCC) |
| FGFR1 - BAG4 | Breast (NOS) |
| | Colon adenocarcinoma (CRC) |
| FGFR1 - SLC12A8 | Lung non-small cell lung carcinoma (NSCLC) (NOS) |
| FGFR1 - TACC1 | Breast invasive lobular carcinoma (ILC) |
| | Breast carcinoma (NOS) |
| FGFR2 - AARSD1 | Intra-hepatic cholangiocarcinoma |
| FGFR2 - APIP | Lung large cell carcinoma |
| FGFR2 -ARMS2 | Stomach adenocarcinoma (NOS) |
| FGFR2 - ATE1 | Breast carcinoma (NOS) |
| | Stomach adenocarcinoma (NOS) |
| FGFR2 - ATF7 | Breast (NOS) |
| FGFR2 - BAIAP2L1 | Esophagus squamous cell carcinoma (SCC) |
| FGFR2 - BICC1 | Unknown primary carcinoma (CUP) (NOS) |
| | Intra-hepatic cholangiocarcinoma |
| | Liver hepatocellular carcinoma (HCC) |
| | Pancreatobiliary carcinoma |
| | Lung adenocarcinoma |
| FGFR2 - CCAR1 | Intra-hepatic cholangiocarcinoma |
| FGFR2 - CCDC6 | Breast (NOS) |
| | Lung non-small cell lung carcinoma (NSCLC) (NOS) |
| FGFR2 - CCSER2 | Breast invasive ductal carcinoma (IDC) |
| FGFR2 - CGNL1 | Intra-hepatic cholangiocarcinoma |
| FGFR2 - CTNNA3 | Intra-hepatic cholangiocarcinoma |
| FGFR2 - EBF1 | Breast invasive ductal carcinoma (IDC) |
| FGFR2 - ERC1 | Extra-hepatic cholangiocarcinoma |
| FGFR2 - FANK1 | Stomach adenocarcinoma (NOS) |
| FGFR2 - CAMK2G | Unknown primary carcinoma (CUP) (NOS) |
| FGFR2 - FLJ40288 | Gastroesophageal junction adenocarcinoma |
| FGFR2 - GUCY2D | Lung non-small cell lung carcinoma (NSCLC) (NOS) |
| FGFR2 - IQGAP2 | Unknown primary carcinoma (CUP) (NOS) |
| FGFR2 - PAWR | Unknown primary carcinoma (CUP) (NOS) |
| | Unknown primary adenocarcinoma |
| FGFR2 -TFEC | Prostate (NOS) |
| FGFR2 - FLNB | Pancreatobiliary carcinoma |
| | Intra-hepatic cholangiocarcinoma |
| FGFR2 - FOXP1 | Ovary (NOS) |
| FGFR2 - GRB2 | Intra-hepatic cholangiocarcinoma |
| | Breast invasive ductal carcinoma (IDC) |
| FGFR2 - IKZF2 | Intra-hepatic cholangiocarcinoma |
| FGFR2 - KCTD1 | Intra-hepatic cholangiocarcinoma |
| FGFR2 - KHDRBS1 | Intra-hepatic cholangiocarcinoma |
| FGFR2 - KIAA1217 | Gallbladder adenocarcinoma |
| | Unknown primary carcinoma (CUP) (NOS) |
| FGFR2 - KIAA1598 | Lung adenocarcinoma |
| | Lung non-small cell lung carcinoma (NSCLC) (NOS) |
| FGFR2 - MACF1 | Pancreatobiliary carcinoma |
| FGFR2 - MYH9 | Unknown primary adenocarcinoma |
| FGFR2 - MYOZ1 | Ovary (NOS) |
| FGFR2 - NOL4 | Intra-hepatic cholangiocarcinoma |
| FGFR2 - NRAP | Breast (NOS) |
| | Lung squamous cell carcinoma (SCC) |
| FGFR2 - NRBF2 | Intra-hepatic cholangiocarcinoma |
| FGFR2 - PCDH15 | Prostate (NOS) |
| FGFR2 - PRKAR1A | Intra-hepatic cholangiocarcinoma |
| FGFR2 - PRRC2A | Breast carcinoma (NOS) |
| FGFR2 - RABGAP1 | Unknown primary adenocarcinoma |
| FGFR2 - RBM20 | Breast carcinoma (NOS) |
| FGFR2 - SCIN | Breast (NOS) |
| FGFR2 - SORBS1 | Intra-hepatic cholangiocarcinoma |
| FGFR2 - SPICE1 | Intra-hepatic cholangiocarcinoma |
| FGFR2 - STAU1 | Intra-hepatic cholangiocarcinoma |
| FGFR2 - STK4 | Colon adenocarcinoma (CRC) |
| FGFR2 - TACC2 | Intra-hepatic cholangiocarcinoma |
| | Pancreatobiliary carcinoma |
| | Unknown primary adenocarcinoma |
| | Lung small cell undifferentiated carcinoma |
| | Breast invasive ductal carcinoma (IDC) |
| | Colon adenocarcinoma (CRC) |
| FGFR2 - TIFA | Breast invasive ductal carcinoma (IDC) |
| FGFR2 - TLK1 | Breast carcinoma (NOS) |
| FGFR2 - TRIM54 | Unknown primary carcinoma (CUP) (NOS) |
| FGFR2 - TRIM8 | Intra-hepatic cholangiocarcinoma |
| FGFR2 - VTI1A | Stomach adenocarcinoma (NOS) |
| FGFR2 - WAC | Lung adenocarcinoma |
| FGFR2 - WARS | Intra-hepatic cholangiocarcinoma |
| | Lung non-small cell lung carcinoma (NSCLC) (NOS) |
| FGFR2 - ZMYM4 | Unknown primary carcinoma (CUP) (NOS) |
| FGFR3 - ADD1 | Lung non-small cell lung carcinoma (NSCLC) (NOS) |
| FGFR3 - CCT5 | Breast (NOS) |
| FGFR3 - CNOT4 | Breast carcinoma (NOS) |
| FGFR3 - IGH | Pancreas (NOS) |
| FGFR3 - TACC3 | Bladder urothelial (transitional cell) carcinoma |
| | Cervix squamous cell carcinoma (SCC) |
| | Esophagus adenocarcinoma |
| | Lung adenocarcinoma |
| | Small intestine adenocarcinoma |
| | Extra-hepatic cholangiocarcinoma |
| | Colorectal (CRC) (NOS) |
| | Lung non-small cell lung carcinoma (NSCLC) (NOS) |
| | Breast (NOS) |
| | Bladder carcinoma (NOS) |
| | Prostate acinar adenocarcinoma |
| | Lung squamous cell carcinoma (SCC) |
| | Colon adenocarcinoma (CRC) |
| | Prostate (NOS) |
| | Cervix adenocarcinoma |
| | Unknown primary squamous cell carcinoma (SCC) |
| | Breast invasive lobular carcinoma (ILC) |
| | Eye intraocular melanoma |
| | Head and neck (NOS) |
| | Bladder adenocarcinoma |
| | Breast invasive ductal carcinoma (IDC) |
| | Lung small cell undifferentiated carcinoma |
| | Colorectal (CRC) (NOS) |
| | Head and neck squamous cell carcinoma (HNSCC) |
| | Breast carcinoma (NOS) |
| | Intra-hepatic cholangiocarcinoma |
| | Kidney (NOS) |
| FGFR3 - TNIP2 | Lung (NOS) |
| FGFR3 - WHSC1 | Prostate acinar adenocarcinoma |
| FOXP1 - FGFR2 | Ovary (NOS) |
| MET - CAPZA2 | Intra-hepatic cholangiocarcinoma |
| | Colon adenocarcinoma (CRC) |
| MET - LDHA | Lung non-small cell lung carcinoma (NSCLC) (NOS) |
| MET - CNTNAP2 | Colon adenocarcinoma (CRC) |
| MET - HBP1 | Prostate (NOS) |
| MET - SPECC1L | Lung adenocarcinoma |
| MET - SNRNP70 | Colon adenocarcinoma (CRC) |
| MET - ST7 | Colon adenocarcinoma (CRC) |
| | Skin melanoma |
| NTRK1 - MEF2D | Prostate (NOS) |
| NTRK3 - ETV6 | Thyroid carcinoma |
| RAD51 - BRAF | Prostate acinar adenocarcinoma |
| RAF1 - POCIA | Prostate acinar adenocarcinoma |
| RAF1 - SYN2 | Colon adenocarcinoma (CRC) |
| RAF1 - ZFYVE20 | Prostate acinar adenocarcinoma |
| RAF1 - TRAK1 | Colon adenocarcinoma (CRC) |
| RET - ADCY1 | Breast carcinoma (NOS) |
| RET - BAIAP2L1 | Breast (NOS) |
| RET - CSGALNACT2 | Esophagus squamous cell carcinoma (SCC) |
| RET - GPHN | Lung non-small cell lung carcinoma (NSCLC) (NOS) |
| RET - NCOA4 | Lung non-small cell lung carcinoma (NSCLC) (NOS) |
| | Colon adenocarcinoma (CRC) |
| | Bladder urothelial (transitional cell) carcinoma |
| | Breast (NOS) |
| | Lung adenocarcinoma |
| | Unknown primary carcinoma (CUP) (NOS) |
| | Unknown primary adenocarcinoma |
| RET - NPY4R | Colon adenocarcinoma (CRC) |
| RET - PAWR | Prostate (NOS) |
| RET - RASGEF1A | Colon adenocarcinoma (CRC) |
| RET - ALOX5 | Prostate acinar adenocarcinoma |
| RET - ARID5B | Prostate (NOS) |
| RET - CCDC6 | Lung adenocarcinoma |
| | Lung non-small cell lung carcinoma (NSCLC) (NOS) |
| | Colon adenocarcinoma (CRC) |
| | Rectum adenocarcinoma (CRC) |
| RET - DHX32 | Lung adenocarcinoma |
| RET - ERC1 | Soft tissue sarcoma (NOS) |
| | Lung non-small cell lung carcinoma (NSCLC) (NOS) |
| RET - KIAA1217 | Colon adenocarcinoma (CRC) |
| | Esophagus adenocarcinoma |
| | Lung non-small cell lung carcinoma (NSCLC) (NOS) |
| RET - KIAA1468 | Lung (NOS) |
| RET - KIF5B | Lung adenocarcinoma |
| | Lung (NOS) |
| | Lung non-small cell lung carcinoma (NSCLC) (NOS) |
| | Breast (NOS) |
| | Lung squamous cell carcinoma (SCC) |
| | Unknown primary adenocarcinoma |
| RET - PDE5A | Intra-hepatic cholangiocarcinoma |
| RET - TRIM24 | Lung adenocarcinoma |
| RET - VCL | Lung adenocarcinoma |
| RET - ZNF365 | Lung (NOS) |
| ROS1 - ABR | Unknown primary neuroendocrine tumor |
| ROS1 - ASCC3 | Breast (NOS) |
| ROS1 - CD74 | Lung adenocarcinoma |
| | Lung non-small cell lung carcinoma (NSCLC) (NOS) |
| ROS1 - ELOVL4 | Prostate (NOS) |
| ROS1 - QKI | Lung adenocarcinoma |
| ROS1 - REV3L | Prostate acinar adenocarcinoma |
| ROS1 - DCBLD1 | Breast invasive ductal carcinoma (IDC) |
| ROS1 - EZR | Lung adenocarcinoma |
| | Lung non-small cell lung carcinoma (NSCLC) (NOS) |
| | Lung (NOS) |
| | Unknown primary carcinoma (CUP) (NOS) |
| ROS1 - GOPC | Colon adenocarcinoma (CRC) |
| | Appendix adenocarcinoma |
| | Ovary serous carcinoma |
| | Liver hepatocellular carcinoma (HCC) |
| | Rectum adenocarcinoma (CRC) |
| | Unknown primary neuroendocrine tumor |
| | Breast invasive ductal carcinoma (IDC) |
| | Melanoma |
| ROS1 - MED23 | Unknown primary carcinoma (CUP) (NOS) |
| ROS1 - MSN | Lung non-small cell lung carcinoma (NSCLC) (NOS) |
| ROS1 - MY05C | Lung adenocarcinoma |
| ROS1 - SLC30A8 | Lung adenocarcinoma |
| ROS1 - SLC38A11 | Prostate (NOS) |
| ROS1 - TLN1 | Lung non-small cell lung carcinoma (NSCLC) (NOS) |
| ROS1 - SLC26A2 | Lung adenocarcinoma |
| ROS1 - SYNGR1 | Lung adenocarcinoma |
| ROS1 - TPD52L1 | Prostate acinar adenocarcinoma |
| ROS1 - TRPC6 | Prostate acinar adenocarcinoma |

TABLE 8
Clinico-genomic characteristics of patients in the studies described herein.

| | All | | | | | | | |
|---|---|---|---|---|---|---|---|---|
| | ctDNA | | | | | | | |
| | cases | | NSCLC | | Breast | | CRC | |
| Total | 36,916 | | 10,754 | | 5,148 | | 2,742 | |
| Cases | | | | | | | | |
| Median age | 68 | | 70 | | 64 | | 63 | |
| (years) | | | | | | | | |
| Gender | 50%:50% | | 47%:53% | | 0.84%:99% | | 57%:43% | |
| (M:F) | | | | | | | | |
| Median | 1.6% | | 1.5% | | 2.2% | | 3.3% | |
| ctDNA | | | | | | | | |
| fraction | | | | | | | | |
| Cases with | 32,492 | (88%) | 9,604 | (89%) | 4,617 | (90%) | 2,471 | (90%) |
| ctDNA | | | | | | | | |
| fraction > | | | | | | | | |
| 0 (%) | | | | | | | | |
| Cases | 30,348 | (93%) | 8,907 | (93%) | 4,413 | (96%) | 2,388 | (97%) |
| with ≥ 1 | | | | | | | | |
| GA (%) | | | | | | | | |
| Avg | 3.3 | | 3.0 | | 3.8 | | 4.2 | |
| GAs/case | | | | | | | | |
| Fusion- | 571 | (1.8%) | 296 | (3.1%) | 44 | (0.95%) | 37 | (1.5%) |
| positive | | | | | | | | |
| cases | | | | | | | | |

| | | | | | Other | |
|---|---|---|---|---|---|---|
| | | | | | cancer | |
| | CUP | | Cholangiocarcinoma | | types | |
| Total | 1,918 | | 901 | | 15,453 | |
| Cases | | | | | | |
| Median age | 69 | | 67 | | 69 | |
| (years) | | | | | | |
| Gender | 50%:50% | | 50%:50% | | 66%:34% | |
| (M:F) | | | | | | |
| Median | 1.6% | | 0.96% | | 1.5% | |
| ctDNA | | | | | | |
| fraction | | | | | | |
| Cases with | 1,667 | (87%) | 767 | (85%) | 13,366 | (86%) |
| ctDNA | | | | | | |
| fraction > | | | | | | |
| 0 (%) | | | | | | |
| Cases | 1,569 | (94%) | 702 | (92%) | 12,369 | (93%) |
| with ≥ 1 | | | | | | |
| GA (%) | | | | | | |
| Avg | 3.1 | | 2.9 | | 3.2 | |
| GAs/case | | | | | | |
| Fusion- | 29 | (1.7%) | 32 | (4.2%) | 133 | (1.0%) |
| positive | | | | | | |
| cases | | | | | | |

GA: genomic alterations; Avg: average, NSCLC: non-small cell lung cancer; CRC: colorectal cancer; CUP: carcinoma of unknown primary.

TABLE 9
ALK fusions detected in ctDNA from
cancer types other than lung cancer.

| Specimen | Disease | ALK fusion |
|---|---|---|
| 1 | Breast | EML4-ALK |
| 2 | Breast | EML4-ALK |
| 3 | Breast | PELI1-ALK |
| 4 | Breast | SLC39A10-ALK |
| 5 | Breast | TFG-ALK |
| 6 | Cholangiocarcinoma | EML4-ALK |
| 7 | CRC | EML4-ALK |
| 8 | CRC | EML4-ALK |
| 9 | CRC | EML4-ALK |
| 10 | CRC | EML4-ALK |
| 11 | CRC | EML4-ALK |
| 12 | CRC | MIPOL1-ALK |
| 13 | CRC | STRN-ALK |
| 14 | Esophagus | ARHGEF7-ALK |
| 15 | Pancreas | ALK-TPM3 |
| 16 | Pancreas | KANK1-ALK |
| 17 | Prostate | AGAP1-ALK |
| 18 | Prostate | ALK-SPINK5 |
| 19 | Prostate | GPR113-ALK |
| 20 | Sarcoma | TPM3-ALK |

CRC: colorectal.

### Conclusions

The results described in this Example show that ctDNA assays reliably identified targetable kinase fusions across cancer types. Thus, genomic profiling of ctDNA may be used to define the molecular drivers of primary tumors and to detect mechanisms of acquired resistance (AR) to therapy.

Analysis of ctDNA identified a large diversity of fusion partners, highlighting the ability to detect diverse kinase fusions with DNA sequencing in ctDNA. In addition, diverse kinase fusions were detected in several tumor types where these are not commonly found, thus demonstrating the importance of genomic profiling to identify uncommon cancer drivers, e.g., by profiling of kinase fusions in ctDNA. Importantly, responses to targeted therapies have been documented in patients with kinase fusions in tumor types where they have not been previously observed, suggesting some kinase fusions may be evaluated for pan-tumor indications (see, e.g., Wang et al. (2017) Oncologist 22:768-773; Singhi et al. (2017) J Natl Compr Canc Netw 15:555-562; Ross et al. (2017) Oncologist 22:1444-1450).

### Example 2: High Concordance for Kinase Fusions Identified in Tissue and Liquid Biopsy Specimens from the Same Patient

This Example describes the results of analyses used to determine the concordance between kinase fusions identified in ctDNA and in tissue biopsies.

### Methods

Hybrid-capture based NGS was performed in FFPE tumor tissue or blood samples as described in Example 1.

For kinase fusion concordance analysis, patients with at least one tumor NGS result and at least one liquid biopsy NGS result (using a blood-based assay for circulating tumor DNA [BBACTD], a blood-based liquid biopsy assay #1 [BBLB1], or a blood-based liquid biopsy assay #2 [BBLB2]; Table 10) in the same disease were identified, both with sufficient tumor content and coverage. Only fusions involving genes and exons covered by the tissue assay and the same exons and genes in the liquid assay were included in the concordance analysis. For patients with >1 liquid specimen available for concordance analysis, the liquid sample with the highest estimated ctDNA fraction was chosen. For patients with >1 tissue specimen available, the tissue specimen collected closest to the time of collection of the liquid specimen was chosen. Manual review of submitted pathology reports was also performed to collect patient treatment history if available.

For sensitivity of kinase fusion detection between tissue and liquid biopsies, percent of positive agreement (PPA) was calculated as true positives/true positives+false negatives, using tissue comprehensive genome profiling (CGP) as the reference. Negative percent agreement (NPA) was calculated as true negatives/true negatives+false positives.

TABLE 10
Liquid biopsy NGS assays included in this study.

| | Blood-based | Blood-based | Blood-based Assay |
|---|---|---|---|
| | Liquid | Liquid | for Circulating |
| | Biopsy Assay # 1 | Biopsy Assay # 2 | Tumor DNA |
| | (BBLB1)^{a} | (BBLB2) | (BBACTD)^{b} |
| Specimen | Peripheral whole | Peripheral whole | Peripheral whole |
| | blood | blood | blood |
| Tumor type | All solid tumors | All solid tumors | All solid tumors |
| # of genes assayed | 324 | 70 | 62 |
| # of genes | 324 | 7 | 6 |
| interrogated for | | | |
| fusions/RE | | | |
| Genome coverage | 0.8 Mb | 0.14 Mb | 0.14 Mb |
| Sequencer | NovaSeq 6000 | HiSeq | HiSeq |
| Variant | SV, CNA, RE, | SV, CNA, RE, MSI- | SV, CNA, RE |
| types/biomarkers | bTMB, MSI-high | high | |
| identified | | | |

SV: short variant point mutations and insertions/deletions;
CNA: copy number alterations;
RE: rearrangements;
bTMB: blood tumor mutational burden;
MSI: microsatellite instability.
^{a}See, e.g., Woodhouse et al., PLoS One. 2020; 15(9):e0237802.
^{b}See, e.g., Clark et al., J Mol Diagn. 2018;20(5):686-702.

### Results

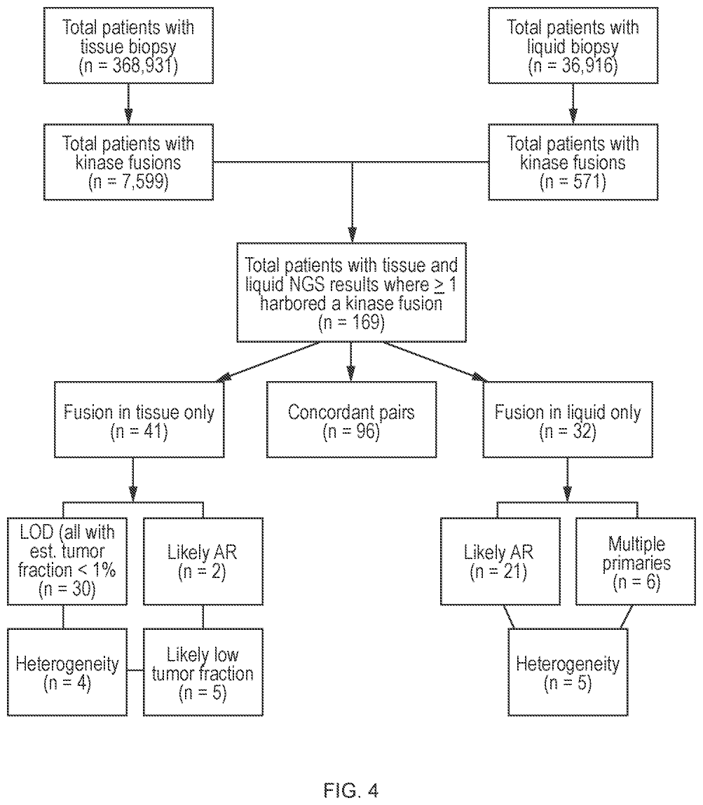

To determine how closely liquid biopsy sequencing of ctDNA recapitulated the kinase fusions identified in tissue samples, 169 cases with both tissue and liquid results available, where at least one sample harbored a kinase fusion, were examined (FIG. 4). Of 137 cases with a fusion detected in tissue, the fusion was also detected in ctDNA in 96 cases (PPA=70%).

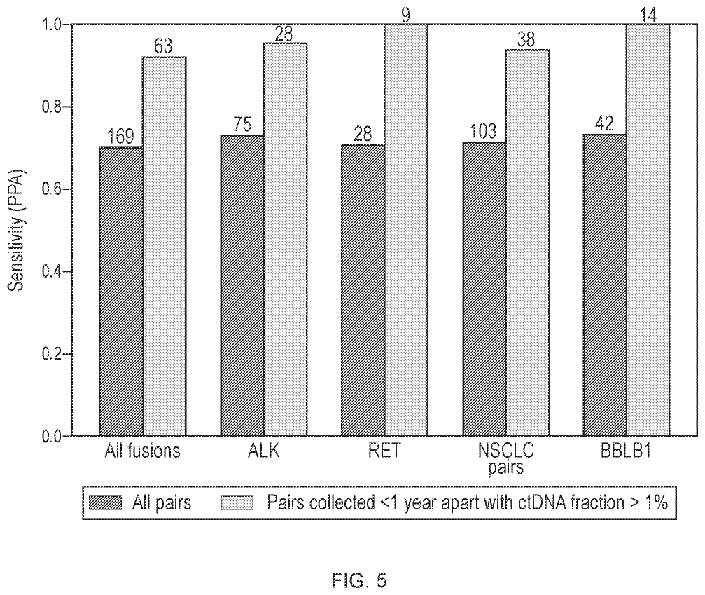

Sensitivity for ALK and RET fusion detection was 73% and 71%, respectively. See, FIG. 5 and Table 11.

ROS1 fusions have been historically challenging to detect in ctDNA, but in this analysis, 10 out of 13 ROS1 fusions identified in tissue samples were also detected in liquid biopsies. In addition, 5 out of 5 ROS1 fusions identified in tissue samples were also detected in liquid biopsies with >1% estimated ctDNA fraction.

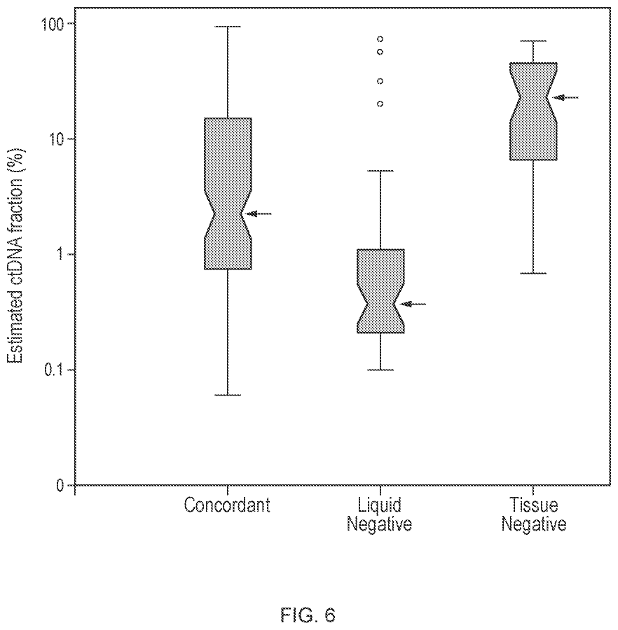

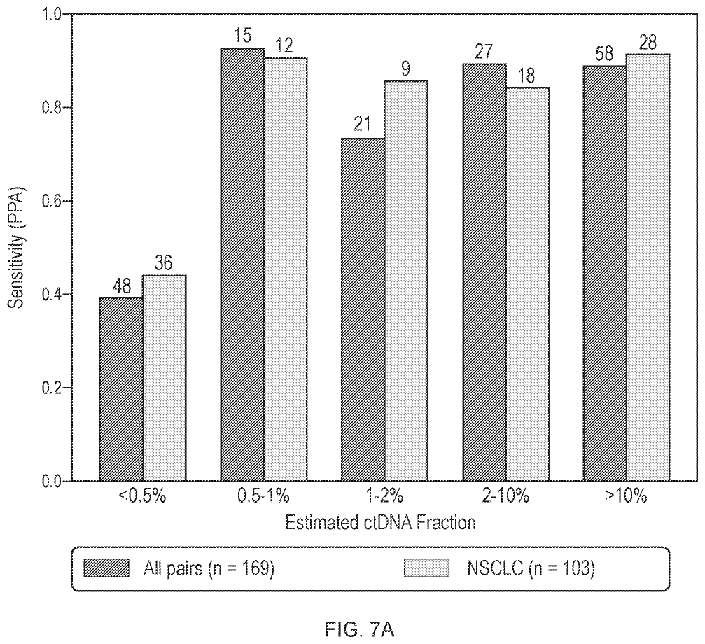

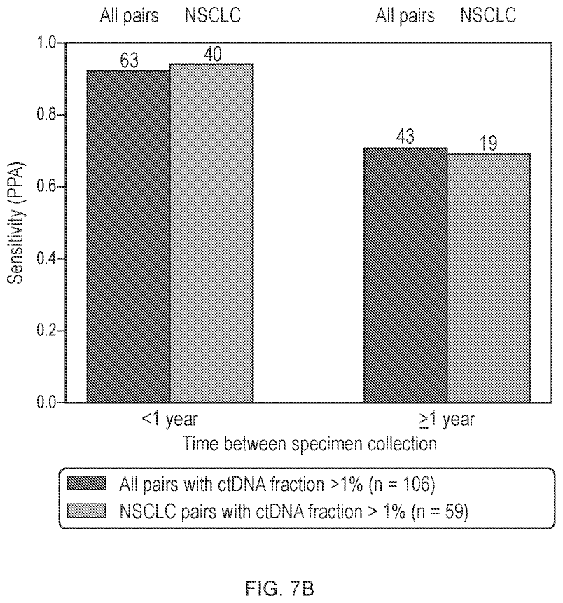

Because variable ctDNA shed is known to impact the sensitivity of ctDNA genotyping (see, e.g., Bieg-Bourne et al. (2020) Mol Oncol 14:1242-1251; Li et al. (2019) J Gastrointest Oncol 10:831-840), whether an estimation of ctDNA fraction influenced sensitivity was evaluated. Median ctDNA fraction was higher in concordant ctDNA samples (2.2%) than in discordant ctDNA samples (0.37%, p<0.001) (FIG. 6). In pairs with tissue-identified fusions, ctDNA detection was improved with higher ctDNA fraction. A direct relationship between PPA and ctDNA fraction of the liquid biopsy specimen was also observed. In liquid specimens with estimated ctDNA fraction <1%, fusions were detected in 32/62 samples with a fusion detected in tissue (PPA=52%), compared to 64/75 samples (PPA=85%) for ctDNA specimens with ctDNA fraction ≥1% (p<0.001) (FIG. 7A).

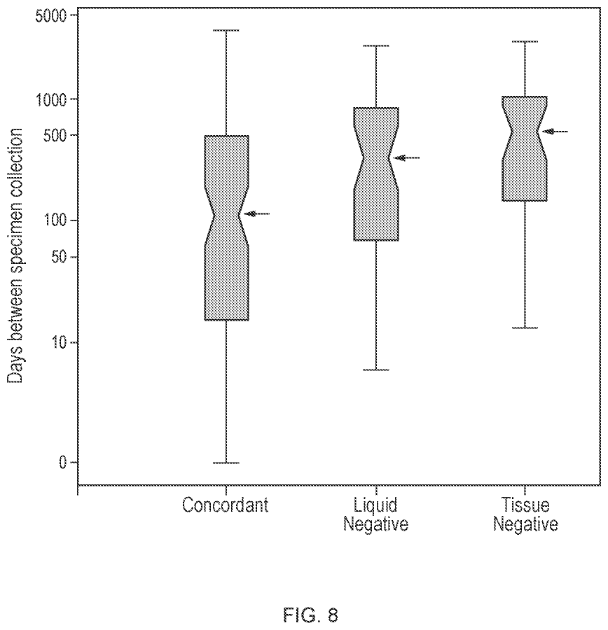

Prolonged time between CGP analyses also has the potential to influence assay concordance (see, e.g., Li et al. (2019) J Gastrointest Oncol 10:831-840). The median time between tissue and liquid sample collection was 206 days (interquartile range: 26-696 days). Among pairs where the estimated plasma ctDNA fraction was >1%, PPA for pairs collected <1 year apart was higher than pairs collected >1 year apart (92% vs. 71%, p=0.03) (FIG. 7B), and the median time between specimen collection for concordant and discordant pairs was 110 and 426 days, respectively (p<0.001) (FIG. 8). Concordant tissue and liquid results were observed more frequently in instances with less time between collection of tissue and liquid specimens. Among temporally matched pairs collected <1 year apart with ctDNA fraction >1%, fusions were detected in 47/51 samples with a fusion in tissue (PPA=92%) (FIG. 5, Table 11).

TABLE 11
Concordance of fusion detection in paired tissue-liquid specimens.

| | | | Paired samples collected <1 year | |
|---|---|---|---|---|
| Paired | All pairs | | apart with ctDNA fraction >1% | |
| cohort | PPA | NPA | PPA | NPA |
| All fusions | 70% | 99% | 92% | 99% |
| | (96/137) | (4,553/4,585) | (47/51) | (1,837/1,849) |
| ALK fusion+ | 73% | 99% | 96% | 99% |
| | (49/67) | (4,553/4,561) | (23/24) | (1,837/1,841) |
| RET fusion+ | 71% | 99% | 100% | 99% |
| | (17/24) | (4,553/4,557) | (8/8) | (1,837/1,838) |
| NSCLC Only | 71% | 99% | 94% | 99% |
| | (65/91) | (1,286/1,298) | (31/33) | (618/623) |
| BBLB1 Only | 73% | 99% | 100% | 99% |
| | (22/30) | (978/990) | (9/9) | (396/401) |

PPA: percent of positive agreement;
NPA: negative percent agreement;
NSCLC: non-small cell lung cancer;
BBLB1: Blood-based Liquid Biopsy Assay # 1.
Disease and kinase-specific subsets with >20 paired specimens are shown.

Next, 41 cases with a fusion detected in tissue but not in liquid were further analyzed (FIG. 4). 30/41 fusions (73%) had likely impaired sensitivity for fusion detection with an estimated tumor fraction of <1%. 5/41 fusions (12%) were absent in samples with suspected false positive estimated tumor fractions of >1% due to use of older assays or presence of possible clonal hematopoiesis. 4/41 discordant fusions (9.8%) were likely due to intratumoral or temporal heterogeneity. 3 of these fusions were detected at low read count (<70 reads) in high tumor content tissue specimens (>50% tumor) and thus may be subclonal events beneath the limit of detection in the liquid biopsy. Furthermore, 2 fusions were not detected in liquid specimens collected >3 years after the primary tissue biopsy. 2/41 fusions (4.9%) were putative acquired resistance (AR) mechanisms detected in tissue after a primary EGFR-mutated liquid specimen (Table 12).

TABLE 12
Fusions detected in tissue biopsy only as
likely acquired resistance mechanisms.

| | | | | | Pre-tissue |
|---|---|---|---|---|---|
| | | Days | | | biopsy |
| | | between | Primary | Secondary | treatment |
| | | specimen | liquid | tissue | and |
| Patient | Disease | collection | biopsy | biopsy | response |
| 1 | NSCLC | 732 | EGFR | EGFR ex19del | Osimertinib |
| | | | ex19del | | |
| | | | EGFR | EGFR T790M | |
| | | | T790M | EML4-ALK fusion | |
| 2 | NSCLC | 1,702 | EGFR | EGFR L858R | NA |
| | | | L858R | | |
| | | | EGFR | EGFR T790M | |
| | | | T790M | CCDC6-RET fusion | |

NA: not available;
NSCLC: non-small cell lung cancer.

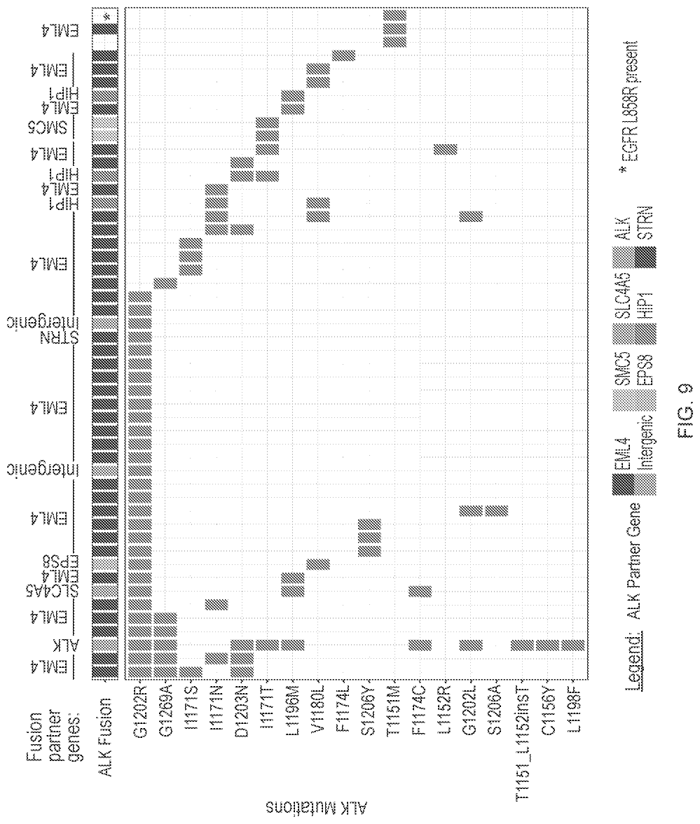

To better understand the risk of false negative results for kinase fusions, results were queried for cases harboring ALK resistance mutations, which should invariably be detected in the presence of a driver ALK fusion. Some reports in the literature have described recurring cases with ALK resistance mutations detected in ctDNA in the absence of the kinase fusion, suggesting gaps in detection of the required prior driver fusion events (see, e.g., Aggarwal et al. (2019) JAMA Oncol 5:173-180; Odegaard et al. (2018) Clin Cancer Res 24:3539-3549; McCoach et al. (2018) Clin Cancer Res 24:2758-2570). 50 cases with a suspected ALK resistance mutation detected (variant allele frequency (VAF) 0.10%-97%) were identified, and in 48 (96%), a corresponding ALK kinase fusion was also identified (FIG. 9 and Table 13). In one case where a fusion was not detected, an EGFR driver mutation was detected, potentially suggesting a subclonal ALK rearrangement event.

TABLE 13
ALK fusions identified in ctDNA specimens harboring known ALK resistance mutations.

| | | Estimated | | ALK resistance | | Other drivers |
|---|---|---|---|---|---|---|
| Specimen | Assay | tumor fraction | ALK fusion | mutations (VAF) | | present (VAF) |
| 1 | BBLB1 | 0.28% | EML4-ALK | G1202R | (0.28%) | |
| 2 | BBLB2 | 0.40% | EML4-ALK | G1269A | (0.28%) | |
| 3 | BBLB2 | 0.45% | EML4-ALK | G1202R | (0.45%) | |
| 4 | BBACTD | 0.50% | EML4-ALK | I1171S | (0.24%) | |
| 5 | BBLB1 | 0.56% | ALK-N/A | G1202R | (0.15%) | |
| 6 | BBLB1 | 0.59% | EML4-ALK | D1203N | (0.26%) | |
| | | | | I1171N | (0.24%) | |
| 7 | BBLB2 | 0.72% | EML4-ALK | G1202R | (0.72%) | |
| | | | | G1269A | (0.70%) | |
| 8 | BBACTD | 0.78% | SMC5-ALK | I1171T | (0.53%) | |
| 9 | BBLB1 | 0.85% | EML4-ALK | G1202R | (0.63%) | |
| 10 | BBACTD | 1.0% | EML4-ALK | G1202R | (0.42%) | |
| | | | | L1196M | (0.24%) | |
| 11 | BBACTD | 1.0% | EML4-ALK | G1202R | (0.68%) | |
| 12 | BBACTD | 1.2% | EML4-ALK | G1202R | (0.38%) | |
| 13 | BBLB1 | 1.3% | EML4-ALK | V1180L | (0.53%) | |
| 14 | BBLB2 | 1.4% | EML4-ALK | G1202R | (1.3%) | |
| 15 | BBACTD | 1.4% | EML4-ALK | G1202R | (0.25%) | |
| 16 | BBLB1 | 1.8% | EML4-ALK | F1174L | (0.30%) | |
| 17 | BBLB1 | 2.6% | EML4-ALK | G1202R | (1.1%) | |
| 18 | BBLB1 | 3.1% | EPS8-ALK | V1180L | (1.8%) | |
| | | | | G1202R | (0.28%) | |
| 19 | BBLB1 | 3.2% | EML4-ALK | I1171S | (3.2%) | |
| 20 | BBLB2 | 3.2% | EML4-ALK | S1206Y | (0.34%) | |
| | | | | G1202R | (0.19%) | |
| 21 | BBLB1 | 3.4% | EML4-ALK | G1202R | (0.21%) | |
| | | | | S1206Y | (0.19%) | |
| 22 | BBLB2 | 3.9% | EML4-ALK | G1202R | (0.19%) | |
| 23 | BBACTD | 4.0% | EML4-ALK | G1202R | (0.64%) | |
| 24 | BBACTD | 4.6% | — | T1151M | (4.6%) | |
| 25 | BBLB2 | 5.3% | EML4-ALK | S1206Y | (0.49%) | |
| | | | | G1202R | (0.39%) | |
| 26 | BBLB2 | 5.9% | EML4-ALK | G1202R | (3.7%) | |
| | | | | G1269A | (3.4%) | |
| 27 | BBLB1 | 6.1% | ALK-SLC4A5 | G1202R | (2.5%) | |
| | | | | L1196M | (1.9%) | |
| | | | | F1174C | (0.23%) | |
| 28 | BBACTD | 6.5% | EML4-ALK | T1151M | (2.9%) | |
| 29 | BBLB1 | 7.7% | EML4-ALK | L1152R | (4.1%) | KRAS A146P |
| | | | | I1171T | (0.20%) | (7.7%) |
| 30 | BBACTD | 10% | EML4-ALK | G1202R | (3.2%) | |
| 31 | BBLB1 | 11% | EML4-ALK | I1171S | (5.0%) | |
| 32 | BBACTD | 11% | EML4-ALK | I1171N | (1.9%) | |
| | | | | G1202R | (0.88%) | |
| 33 | BBACTD | 11% | EML4-ALK | D1203N | (1.7%) | EGFR R748K |
| | | | | | | (1.2%) |
| 34 | BBLB1 | 11% | EML4-ALK | G1269A | (2.4%) | |
| | | | | I1171S | (2.4%) | |
| | | | | G1202R | (2.0%) | |
| | | | | D1203N | (1.7%) | |
| 35 | BBLB2 | 14% | HIP1-ALK | V1180L | (2.4%) | |
| | | | | I1171N | (0.63%) | |
| 36 | BBLB2 | 14% | HIP1-ALK | I1171T | (0.58%) | KRAS G12V |
| | | | | D1203N | (0.42%) | (0.23%) |
| 37 | BBLB2 | 15% | EML4-ALK | V1180L | (1.4%) | |
| | | | | G1202L | (0.94%) | |
| | | | | I1171N | (0.22%) | |
| 38 | BBACTD | 15% | SMC5-ALK | I1171T | (6.9%) | |
| 39 | BBLB2 | 16% | EML4-ALK | L1196M | (0.10%) | |
| 40 | BBLB2 | 17% | EML4-ALK | V1180L | (6.8%) | EGFR D761N |
| | | | | | | (3.5%) |
| 41 | BBACTD | 18% | EML4-ALK | I1171N | (2.7%) | |
| 42 | BBLB2 | 19% | EML4-ALK | G1202L | (6.9%) | |
| | | | | S1206A | (2.9%) | |
| | | | | G1202R | (2.9%) | |
| 43 | BBLB1 | 21% | ALK-ALK | G1202R | (3.9%) | |
| | | | | I1171T | (3.1%) | |
| | | | | G1202L | (1.1%) | |
| | | | | L1198F | (1.0%) | |
| | | | | G1269A | (0.61%) | |
| | | | | G1202R | (0.59%) | |
| | | | | F1174C | (0.36%) | |
| | | | | L1196M | (0.32%) | |
| | | | | C1156Y | (0.29%) | |
| | | | | T1151_L1152insT | (0.16%) | |
| | | | | D1203N | (0.15%) | |
| 44 | BBLB2 | 23% | EML4-ALK | G1202R | (9.9%) | |
| | | | STRN-ALK | | | |
| 45 | BBLB2 | 25% | — | T1151M | (0.22) | EGFR L858R |
| | | | | | | (25%) |
| 46 | BBACTD | 33% | EML4-ALK | I1171N | (11%) | |
| | | | | D1203N | (6.0%) | |
| | | | | G1269A | (4.8%) | |
| | | | | G1202R | (1.0%) | |
| 47 | BBLB2 | 61% | ALK-N/A | G1202R | (0.73%) | |
| 48 | BBACTD | 89% | EML4-ALK | G1202R | (89%) | |
| 49 | BBACTD | 95% | HIP1-ALK | L1196M | (45%) | |
| 50 | BBACTD | 97% | EML4-ALK | G1202R | (97%) | |

VAF: variant allele frequency;
BBACTD: Blood-based Assay for Circulating Tumor DNA;
BBLB2: Blood-based Liquid Biopsy Assay # 2;
BBLB1: Blood-based Liquid Biopsy Assay # 1.

### Conclusions

ctDNA fraction can be impacted by factors that influence tumor DNA shedding including tumor type, location, size, stage or vascularity, which can affect the accessibility of the tumor to circulation (see, e.g., Bettegowda et al. (2014) Sci Transl Med 6: 224ra24; Jahr et al. (2001) Cancer Res 61:1659-1665; Ignatiadis et al. (2014) Ann Oncol 25:2304-2313). These biological factors can affect the release of tumor DNA in the blood, potentially impacting the representation and detectability of kinase fusions in ctDNA (see, e.g., Grasselli et al. (2017) Ann Oncol 6:1294-1301; Bando et al. (2019) Br J Cancer 10:982-986). However, the results described in this Example showed that genomic profiling of ctDNA closely recapitulated the results of tissue-based testing, and the majority of discordances could be attributed to a combination of biological and/or analytical factors (e.g., low ctDNA fraction, temporal factors, evolution of acquired resistance (AR), subclonal mutations, and multiple primary tumors).

### Example 3: Acquired Kinase Fusions in ctDNA May Mediate Drug Resistance

This Example describes an analysis of kinase fusions that were detected only in ctDNA and not in tissue biopsies from the same patient.

### Methods

Hybrid-capture based NGS was performed in FFPE tumor tissue or blood samples as described in Example 1.

### Results

Thirty-two specimens from patients in which a kinase fusion was detected exclusively in liquid biopsy were evaluated (FIG. 4). In 6/32 (19%) of these cases, comparison of the tissue and liquid results raised the possibility of multiple primary tumors as a potential mechanism for discordance. For example, one subject with a clinical diagnosis of perineal sarcoma had tissue testing that revealed an EWSR1-ATF1 fusion, a driver event strongly associated with clear cell sarcomas, whereas liquid testing showed a high confidence EML4-ALK fusion, which is most frequently found in NSCLC.

Four of the 32 fusions (13%) were observed at low read count (<10 reads) and may represent intratumoral heterogeneity captured by liquid biopsy. 1/32 fusions (3.1%) was a RET fusion in a prostate cancer liquid specimen collected >2 years after the primary tissue specimen, and could represent temporal heterogeneity and evolution to neuroendocrine prostate cancer (see, e.g., VanDeusen et al. (2020) Mol Cancer Res 18:1176-1188).

In the remaining 21/32 cases (66%), there was evidence that the fusion represented putative acquired resistance based on the detection of a co-occurring EGFR (n=10) or other driver mutation (n=3), or other co-occurring established resistance mechanisms (n=8) (Table 14). Such acquired kinase fusions have been described as resistance mechanisms to targeted therapies, including EGFR tyrosine kinase inhibitors in NCSLC patients harboring EGFR driver mutations (see, e.g., Xu et al. (2019) Cancer Manag Res 11:6343-6351; Piotrowska et al. (2018) Cancer Discov 8:1529-1539; Schrock et al. (2018) J Thorac Oncol 13:1312-1323; Schrock et al. (2019) J Thorac Oncol 14:255-264).

As shown in Table 14 (lines 1-5, 7, 8, 10, 11, and 13-21), in 18/21 of these cases, the tissue biopsy pre-dated the ctDNA specimen collection date (median 813-day difference, range 75-2,959). The remaining 3/21 cases had a liquid specimen collected prior to the paired tissue biopsy (Table 14, lines 6, 9, and 12) and harbored kinase fusions that co-occurred with an activating driver alteration that was detected by both tissue and liquid CGP. These fusions were all detected at low read count (range 3-62) and may represent subclonal acquired resistance that was unable to be detected by tissue CGP.

As shown in Table 14 (lines 1-10), 10/21 cases were NSCLC specimens, where an activating EGFR mutation co-occurred with a range of ctDNA-detected kinase fusions, including ALK (n=4), FGFR3 (n=3), RET (n=2), and FGFR2 (n=1) (see, e.g., Schrock et al. (2018) J Thorac Oncol 13:1312-1323; Leonetti et al. (2019) Br J Cancer 121:725-737). Importantly, some of these acquired fusions were detected after treatment with first-, second-, or third-generation EGFR TKIs (Schrock et al. Receptor Tyrosine Kinase Fusions and BRAF Kinase Fusions are Rare but Actionable Resistance Mechanisms to EGFR Tyrosine Kinase Inhibitors. J Thorac Oncol. 2018). Moreover, these fusions frequently co-occurred with other mechanisms of resistance. For example, two patients harbored well-characterized EGFR resistance mutations T790M with and without C797G, which co-occurred with FGFR3 and FGFR2 fusions, respectively. One sample additionally acquired a BRAF V600E mutation co-occurring with an FGFR3 fusion in the post-treatment sample. 7/21 of these cases were KRAS wild-type (WT) colorectal carcinoma (CRC) samples. ALK, BRAF, EGFR, FGFR3, MET, and RAF1 fusions were identified as possible acquired resistance to anti-EGFR therapy and occurred alongside other putative resistance alterations including EGFR alterations and RAS mutations (Table 14, lines 11-13, 15-17, and 19) (see, e.g., Clifton et al. (2019) JCO Precis Oncol 3; Stangl et al. (2020) Mol Cancer Res 18:537-548; Zhao et al. (2017) Oncotarget 8:3980-4000). Two of the 21 specimens were KRAS-mutated CRC samples with possible acquired BRAF, FGFR3, and RAF1 fusions, which could mediate resistance to regorafenib and adagrasib (Table 14, lines 14, 18). Two of the 21 specimens were breast samples with acquired RET and FGFR3 fusions. (Table 14, lines 20-21). The FGFR3 fusion was present in a hormone receptor positive specimen and seen alongside two ESR1 mutations, and may represent heterogeneous acquired resistance to hormonal therapy (see, e.g., Liu et al. (2020) Cancer Research 80:5280). The RET fusion was observed alongside an activating PIK3CA mutation and may similarly confer resistance to PI3K targeted therapy. In addition, in 6 cases with ctDNA-only fusions, the kinase fusions were indicative of a possible separate primary, highlighting that ctDNA testing may provide added value beyond tissue testing to detect multiple primary tumors co-existing in a single patient (FIG. 4).

Collectively, these findings highlight the clinical validity of liquid biopsy CGP to identify potentially targetable mechanisms of AR.

TABLE 14
Fusions detected in ctDNA but not in tissue may
represent acquired resistance mechanisms.

| | | Days | | | Pre-liquid |
|---|---|---|---|---|---|
| | | between | | | biopsy |
| | | specimen | | | treatment and |
| Case | Disease | collection | Tissue biopsy | Liquid biopsy | response |
| 1 | NSCLC | 75 | EGFR | EGFR ex19del | Gefitinib |
| | | | ex19del, | EML4-ALK fusion | |
| | | | C797S | | |
| 2 | NSCLC | 196 | EGFR L858R | EGFR L858R | NA |
| | | | | EML4-ALK fusion | |
| 3 | NSCLC | 775 | EGFR ex19del | EGFR ex19del | NA |
| | | | | EML4-ALK fusion | |
| 4 | NSCLC | 894 | EGFR L858R | EGFR L858R | Erlotinib (12 mos) - |
| | | | | PLEKHA7-ALK | PR |
| | | | | fusion | Afatinib (2 mos) |
| | | | | | Osimertinib (12 mos) - |
| | | | | | PR * |
| 5 | NSCLC | 454 | EGFR L858R, | EGFR L858R, | Erlotinib |
| | | | L833V | L833V, T790M | |
| | | | | FGFR2-CCDC6 | |
| | | | | fusion | |
| 6^{#} | NSCLC | 133 | EGFR ex19del | EGFR ex19del | NA |
| | | | | FGFR3-TACC3 | |
| | | | | fusion | |
| 7 | NSCLC | 426 | EGFR L858R, | EGFR L858R | Afatinib + cetuximab |
| | | | E709K | FGFR3-TACC3 | (10 mos) - SD * |
| | | | | fusion | |
| 8 | NSCLC | 464 | EGFR | EGFR ex19del, | Osimertinib (7 mos) - |
| | | | ex19del, | T790M, C797G | SD |
| | | | T790M | BRAF V600E | |
| | | | | FGFR3-ADD1 | |
| | | | | fusion | |
| 9^{#} | NSCLC | 13 | EGFR ex19del | EGFR ex19del | NA |
| | | | | ERC1-RET fusion | |
| 10 | NSCLC | 686 | EGFR | EGFR ex19del | Osimertinib (8 mos) |
| | | | ex19del, | NCOA4-RET | |
| | | | T790M | fusion | |
| 11 | CRC | 850 | KRAS WT | EGFR amp | FOLFOX, |
| | | | | MAP2K1 | Capecitabine, Lonsurf, |
| | | | | I103_K104del | Ramucirumab, |
| | | | | NF1 truncation | Bevacizumab, |
| | | | | EML4-ALK fusion | Panitumumab |
| 12^{#} | CRC | 31 | KRAS WT | EGFR V441G, | NA |
| | | | MET amp | G465E/R | |
| | | | | NRAS G13D, Q61K | |
| | | | | KRAS G12C | |
| | | | | ZC3HAV1-BRAF | |
| | | | | fusion | |
| 13 | CRC | 615 | KRAS WT | EGFR S492R | NA |
| | | | | KRAS G12V, Q61H | |
| | | | | NRAS Q61K/L | |
| | | | | MAP2K1 Q58del, | |
| | | | | I111T | |
| | | | | MAP2K2 F57V | |
| | | | | NF1 F945fs*9 | |
| | | | | MKRN1-BRAF | |
| | | | | fusion | |
| 14 | CRC | 1,014 | KRAS G13D | KRAS G13D | FOLFOX, 5FU |
| | | | | DENND2A-BRAF | maintenance, FOLFIRI, |
| | | | | fusion | regorafenib |
| 15 | CRC | 2,959 | KRAS WT | NRAS Q61K | NA |
| | | | | TRIM24-BRAF | |
| | | | | fusion | |
| 16 | CRC | 150 | KRAS WT | KRAS G12A, Q61H | NA |
| | | | | NRAS G12D | |
| | | | | MAP2K1 | |
| | | | | E102_1103del | |
| | | | | PDE7A-EGFR | |
| | | | | fusion | |
| 17 | CRC | 854 | KRAS WT | BRAF V600E | FOLFIRI + |
| | | | | EGFR V441G, | bevacizumab (4 mos) |
| | | | | S492R | FOLFIRI + cetuximab |
| | | | | HRAS Q61L | (6 mos) |
| | | | | MAP2K1 K57T, | FOLFOX + |
| | | | | E102_I103del | bevacizumab (11 mos) |
| | | | | NRAS Q61K | pembrolizumab + |
| | | | | FGFR3-TACC3 | regorafenib |
| | | | | fusion | |
| | | | | GOLGA3-BRAF | |
| | | | | fusion | |
| | | | | SYN2-RAF1 fusion | |
| 18\hat{ } | CRC | 1,912 | KRAS G12C | KRAS G12C, G13D | Adagrasib |
| | | | | MAP2K1 | Adagrasib + cetuximab |
| | | | | E102_I103del | |
| | | | | NRAS Q61K | |
| | | | | AKAP9-BRAF | |
| | | | | fusion | |
| | | | | FGFR3-TACC3 | |
| | | | | fusion | |
| | | | | RAF1-TRAK1 | |
| | | | | fusion | |
| | | | | RAF1-CCDC176 | |
| | | | | fusion | |
| 19 | CRC | 1,236 | KRAS WT | KRAS Q61H | NA |
| | | | | EGFR amp | |
| | | | | MET-CAPZA2 | |
| | | | | fusion | |
| 20 | Breast | 1,269 | ER+/PR+ | ESR1 Y537N, | Everolimus, |
| | | | | D538G | denosumab, fulvestrant |
| | | | | AKT1 E17K | |
| | | | | FGFR3-TACC3 | |
| | | | | fusion | |
| 21 | Breast | 1,591 | PIK3CA | PIK3CA E542K | NA |
| | | | E542K | ESR1 E380Q | |
| | | | | KRAS G12C | |
| | | | | PTEN S59*, M134I | |
| | | | | BAIAP2L1-RET | |
| | | | | fusion | |

NA: not available;
PR: partial response;
SD: stable disease;
amp: amplification;
WT: wild type;
ER+/PR+: estrogen receptor positive/progesterone receptor positive;
NSCLC: non-small cell lung cancer;
CRC: colorectal.
^{#}These patients had their liquid specimen collected before the tissue specimen and the fusions may represent subclonal resistance not captured in the subsequent tissue biopsy. All other patient pairs had the fusion-negative tissue collected first.
*Schrock et al. Receptor Tyrosine Kinase Fusions and BRAF Kinase Fusions are Rare but Actionable Resistance Mechanisms to EGFR Tyrosine Kinase Inhibitors. J Thorac Oncol. 2018.
\hat{ }Awad et al. Acquired Resistance to KRAS G12C Inhibition in Cancer. New England Journal of Medicine, 2021

### Conclusions

Kinase fusions found only by liquid biopsies, and not in the tissue testing, were often associated with acquired resistance. Thus, liquid biopsies can accurately capture dynamic changes in a patient's tumor over time and illuminate the challenges of overcoming resistance to targeted therapy. Taken together, these results support the clinical validity of ctDNA profiling for both primary and recurrent tumors.

1-7. (canceled)

8. A method of treating or delaying progression of cancer, comprising:
(a) acquiring knowledge of a fusion nucleic acid molecule, or a fusion polypeptide encoded by the fusion nucleic acid molecule, in a sample from an individual having a cancer, wherein:
(i) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or
(ii) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2; and
(b) responsive to said knowledge, administering to the individual an effective amount of a treatment that comprises an anti-cancer therapy.

9. A method of treating or delaying progression of cancer, comprising administering to an individual having cancer an effective amount of a treatment that comprises an anti-cancer therapy, wherein the anti-cancer therapy is administered responsive to acquiring knowledge of a fusion nucleic acid molecule, or a fusion polypeptide encoded by the fusion nucleic acid molecule, in a sample from the individual, wherein:
(a) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or
(b) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2.

10-48. (canceled)

49. A method of treating or delaying progression of cancer, comprising:
(a) detecting in a sample from an individual having a cancer a fusion nucleic acid molecule, or a fusion polypeptide encoded by the fusion nucleic acid molecule, wherein:
(i) the fusion nucleic acid molecule is an ALK, BRAF, EGFR, ERBB2, FGFR1, FGFR2, FGFR3, MET, RAF1, RET, or ROS1 fusion nucleic acid molecule listed in Table 1, or
(ii) the fusion nucleic acid molecule is an ALK, BRAF, ERBB2, FGFR1, FGFR2, FGFR3, MET, NTRK1, RAF1, RET, or ROS1 fusion nucleic acid molecule listed in Table 2, and the cancer is the cancer corresponding to the ALK, BRAF, ERBB2, FGFR1, FGFR2, FGFR3, MET, NTRK1, RAF1, RET, or ROS1 fusion nucleic acid molecule as listed in Table 2; and
(b) administering to the individual an effective amount of a treatment that comprises an anti-cancer therapy.

50. The method of claim 49, wherein the fusion nucleic acid molecule is a ROS1 fusion nucleic acid molecule listed in Table 1, comprising or resulting from a Breakpoint 1 and/or a Breakpoint 2 corresponding to the ROS1 fusion nucleic acid molecule as listed in Table 3.

51-53. (canceled)

54. The method of claim 49, wherein the fusion nucleic acid molecule is a ROS1 fusion nucleic acid molecule listed in Table 1, and wherein the cancer is a B cell cancer, a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

55-57. (canceled)

58. The method of claim 49, wherein:
(i) the fusion nucleic acid molecule is a ROS1 fusion nucleic acid molecule listed in Table 2, and the cancer is the cancer corresponding to the ROS1 fusion nucleic acid molecule as listed in Table 2; and
(ii) the fusion nucleic acid molecule comprises or results from a Breakpoint 1 and/or a Breakpoint 2 corresponding to the ROS1 fusion nucleic acid molecule as listed in Table 6.

59-319. (canceled)

320. The method of claim 49, wherein the fusion nucleic acid molecule is a ROS1 fusion nucleic acid molecule as listed in any of Tables 1-6.

321. (canceled)

322. The method of claim 320, wherein the ROS1 fusion nucleic acid molecule encodes a ROS1 fusion polypeptide that comprises a ROS1 kinase domain, or a fragment of a ROS1 kinase domain having ROS1 kinase activity.

323-325. (canceled)

326. The method of claim 320, further comprising acquiring knowledge of or detecting, in a sample from the individual, the presence of a PIK3CA gene mutation; optionally wherein the mutation results in an E545K amino acid substitution in an encoded PIK3CA polypeptide.

327-329. (canceled)

330. The method of claim 320, wherein the anti-cancer therapy is a ROS1-targeted therapy that comprises a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for a ROS1-rearranged cancer, a ROS1-targeted therapy being tested in a clinical trial, a treatment for ROS1-rearranged cancer being tested in a clinical trial, or any combination thereof.

331. (canceled)

332. The method of claim 330, wherein the ROS1-targeted therapy is a tyrosine kinase inhibitor, a multi-kinase inhibitor, or a ROS1-specific inhibitor.

333-334. (canceled)

335. The method of claim 330, wherein the ROS1-targeted therapy comprises one or more of crizotinib, lorlatinib, TQ-B3139, repotrectinib (TPX-0005), brigatinib, cabozantinib, ceritinib, or entrectinib.

336-361. (canceled)

362. The method of claim 49, wherein the sample is from a tumor biopsy, tumor specimen, or circulating tumor cell.

363. The method of claim 49, wherein the sample is a liquid biopsy sample and comprises blood, plasma, cerebrospinal fluid, sputum, stool, urine, or saliva.

364. The method of claim 49, wherein the sample comprises cells and/or nucleic acids from the cancer.

365-367. (canceled)

368. The method of claim 49, comprising detecting the fusion nucleic acid molecule or the fusion polypeptide encoded by the fusion nucleic acid molecule in a tissue biopsy sample, in a liquid biopsy sample, or in both a tissue biopsy sample and a liquid biopsy sample, from the individual.

369. (canceled)

370. The method of claim 49, wherein the detecting comprises detecting a fragment of the fusion nucleic acid molecule comprising a breakpoint or fusion junction.

371. The method of claim 49, wherein the fusion nucleic acid molecule is detected in the sample by one or more of: a nucleic acid hybridization assay, an amplification-based assay, a polymerase chain reaction-restriction fragment length polymorphism (PCR-RFLP) assay, real-time PCR, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), mass-spectrometric genotyping, or sequencing.

372-374. (canceled)

375. The method of claim 49, further comprising selectively enriching for one or more nucleic acids in the sample comprising nucleotide sequences corresponding to the fusion nucleic acid molecule; wherein the selectively enriching produces an enriched sample.

376-383. (canceled)

384. The method of claim 49, wherein the individual is a human.

385-413. (canceled)

414. The method of claim 54, wherein the cancer is a lung cancer.

415. The method of claim 414, wherein the lung cancer is lung adenocarcinoma or non-small cell lung carcinoma (NSCLC).


SEQUENCE LISTING (12 sequences)

SEQ ID NO: 1 - DNA, 6240 bp, Homo sapiens
  source 1..6240: mol_type = unassigned DNA
agatgcgatccagcggctctgggggcggcagcggtggtagcagctggtacctcccgccgcctctgttcggagggtcgcggggcaccgaggtgctttccggccgccctctggtcggccacccaaagccgcgggcgctgatgatgggtgaggagggggcggcaagatttcgggcgcccctgccctgaacgccctcagctgctgccgccggggccgctccagtgcctgcgaactctgaggagccgaggcgccggtgagagcaaggacgctgcaaacttgcgcagcgcgggggctgggattcacgcccagaagttcagcaggcagacagtccgaagccttcccgcagcggagagatagcttgagggtgcgcaagacggcagcctccgccctcggttcccgcccagaccgggcagaagagcttggaggagccaaaaggaacgcaaaaggcggccaggacagcgtgcagcagctgggagccgccgttctcagccttaaaagttgcagagattggaggctgccccgagaggggacagaccccagctccgactgcggggggcaggagaggacggtacccaactgccacctcccttcaaccatagtagttcctctgtaccgagcgcagcgagctacagacgggggcgcggcactcggcgcggagagcgggaggctcaaggtcccagccagtgagcccagtgtgcttgagtgtctctggactcgcccctgagcttccaggtctgtttcatttagactcctgctcgcctccgtgcagttgggggaaagcaagagacttgcgcgcacgcacagtcctctggagatcaggtggaaggagccgctgggtaccaaggactgttcagagcctcttcccatctcggggagagcgaagggtgaggctgggcccggagagcagtgtaaacggcctcctccggcgggatgggagccatcgggctcctgtggctcctgccgctgctgctttccacggcagctgtgggctccgggatggggaccggccagcgcgcgggctccccagctgcggggccgccgctgcagccccgggagccactcagctactcgcgcctgcagaggaagagtctggcagttgacttcgtggtgccctcgctcttccgtgtctacgcccgggacctactgctgccaccatcctcctcggagctgaaggctggcaggcccgaggcccgcggctcgctagctctggactgcgccccgctgctcaggttgctggggccggcgccgggggtctcctggaccgccggttcaccagccccggcagaggcccggacgctgtccagggtgctgaagggcggctccgtgcgcaagctccggcgtgccaagcagttggtgctggagctgggcgaggaggcgatcttggagggttgcgtcgggccccccggggaggcggctgtggggctgctccagttcaatctcagcgagctgttcagttggtggattcgccaaggcgaagggcgactgaggatccgcctgatgcccgagaagaaggcgtcggaagtgggcagagagggaaggctgtccgcggcaattcgcgcctcccagccccgccttctcttccagatcttcgggactggtcatagctccttggaatcaccaacaaacatgccttctccttctcctgattattttacatggaatctcacctggataatgaaagactccttccctttcctgtctcatcgcagccgatatggtctggagtgcagctttgacttcccctgtgagctggagtattcccctccactgcatgacctcaggaaccagagctggtcctggcgccgcatcccctccgaggaggcctcccagatggacttgctggatgggcctggggcagagcgttctaaggagatgcccagaggctcctttctccttctcaacacctcagctgactccaagcacaccatcctgagtccgtggatgaggagcagcagtgagcactgcacactggccgtctcggtgcacaggcacctgcagccctctggaaggtacattgcccagctgctgccccacaacgaggctgcaagagagatcctcctgatgcccactccagggaagcatggttggacagtgctccagggaagaatcgggcgtccagacaacccatttcgagtggccctggaatacatctccagtggaaaccgcagcttgtctgcagtggacttctttgccctgaagaactgcagtgaaggaacatccccaggctccaagatggccctgcagagctccttcacttgttggaatgggacagtcctccagcttgggcaggcctgtgacttccaccaggactgtgcccagggagaagatgagagccagatgtgccggaaactgcctgtgggtttttactgcaactttgaagatggcttctgtggctggacccaaggcacactgtcaccccacactcctcaatggcaggtcaggaccctaaaggatgcccggttccaggaccaccaagaccatgctctattgctcagtaccactgatgtccccgcttctgaaagtgctacagtgaccagtgctacgtttcctgcaccgatcaagagctctccatgtgagctccgaatgtcctggctcattcgtggagtcttgaggggaaacgtgtccttggtgctagtggagaacaaaaccgggaaggagcaaggcaggatggtctggcatgtcgccgcctatgaaggcttgagcctgtggcagtggatggtgttgcctctcctcgatgtgtctgacaggttctggctgcagatggtcgcatggtggggacaaggatccagagccatcgtggcttttgacaatatctccatcagcctggactgctacctcaccattagcggagaggacaagatcctgcagaatacagcacccaaatcaagaaacctgtttgagagaaacccaaacaaggagctgaaacccggggaaaattcaccaagacagacccccatctttgaccctacagttcattggctgttcaccacatgtggggccagcgggccccatggccccacccaggcacagtgcaacaacgcctaccagaactccaacctgagcgtggaggtggggagcgagggccccctgaaaggcatccagatctggaaggtgccagccaccgacacctacagcatctcgggctacggagctgctggcgggaaaggcgggaagaacaccatgatgcggtcccacggcgtgtctgtgctgggcatcttcaacctggagaaggatgacatgctgtacatcctggttgggcagcagggagaggacgcctgccccagtacaaaccagttaatccagaaagtctgcattggagagaacaatgtgatagaagaagaaatccgtgtgaacagaagcgtgcatgagtgggcaggaggcggaggaggagggggtggagccacctacgtatttaagatgaaggatggagtgccggtgcccctgatcattgcagccggaggtggtggcagggcctacggggccaagacagacacgttccacccagagagactggagaataactcctcggttctagggctaaacggcaattccggagccgcaggtggtggaggtggctggaatgataacacttccttgctctgggccggaaaatctttgcaggagggtgccaccggaggacattcctgcccccaggccatgaagaagtgggggtgggagacaagagggggtttcggagggggtggaggggggtgctcctcaggtggaggaggcggaggatatataggcggcaatgcagcctcaaacaatgaccccgaaatggatggggaagatggggtttccttcatcagtccactgggcatcctgtacaccccagctttaaaagtgatggaaggccacggggaagtgaatattaagcattatctaaactgcagtcactgtgaggtagacgaatgtcacatggaccctgaaagccacaaggtcatctgcttctgtgaccacgggacggtgctggctgaggatggcgtctcctgcattgtgtcacccaccccggagccacacctgccactctcgctgatcctctctgtggtgacctctgccctcgtggccgccctggtcctggctttctccggcatcatgattgtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagagatctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtggaggagagagaggcaatggctccttcacaaaccagagaccaaatgtcacgttttgttttgtgccaacctattttgaagtaccaccaaaaaagctgtattttgaaaatgctttagaaaggttttgagcatgggttcatcctattctttcgaaagaagaaaatatcataaaaatgagtgataaatacaaggcccagatgtggttgcataaggtttttatgcatgtttgttgtatacttccttatgcttctttcaaattgtgtgtgctctgcttcaatgtagtcagaattagctgcttctatgtttcatagttggggtcatagatgtttccttgccttgttgatgtggacatgagccatttgaggggagagggaacggaaataaaggagttatttgtaatgactaa

SEQ ID NO: 2 - DNA, 6459 bp, Homo sapiens
  source 1..6459: mol_type = unassigned DNA
cttcccccaatcccctcaggctcggctgcgcccggggccgcgggccggtacctgaggtggcccaggcgccctccgcccgcggcgccgcccgggccgctcctccccgcgccccccgcgccccccgctcctccgcctccgcctccgcctccgcctcccccagctctccgcctcccttccccctccccgcccgacagcggccgctcgggccccggctctcggttataagatggcggcgctgagcggtggcggtggtggcggcgcggagccgggccaggctctgttcaacggggacatggagcccgaggccggcgccggcgccggcgccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctccaggacctcagcgagaaaggaagtcatcttcatcctcagaagacaggaatcgaatgaaaacacttggtagacgggactcgagtgatgattgggagattcctgatgggcagattacagtgggacaaagaattggatctggatcatttggaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaaacttatagatattgcacgacagactgcacagggcatggattacttacacgccaagtcaatcatccacagagacctcaagagtaataatatatttcttcatgaagacctcacagtaaaaataggtgattttggtctagctacagtgaaatctcgatggagtgggtcccatcagtttgaacagttgtctggatccattttgtggatggcaccagaagtcatcagaatgcaagataaaaatccatacagctttcagtcagatgtatatgcatttggaattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccactgaaacaaatgagtgagagagttcaggagagtagcaacaaaaggaaaataaatgaacatatgtttgcttatatgttaaattgaataaaatactctctttttttttaaggtgaaccaaagaacacttgtgtggttaaagactagatataatttttccccaaactaaaatttatacttaacattggatttttaacatccaagggttaaaatacatagacattgctaaaaattggcagagcctcttctagaggctttactttctgttccgggtttgtatcattcacttggttattttaagtagtaaacttcagtttctcatgcaacttttgttgccagctatcacatgtccactagggactccagaagaagaccctacctatgcctgtgtttgcaggtgagaagttggcagtcggttagcctgggttagataaggcaaactgaacagatctaatttaggaagtcagtagaatttaataattctattattattcttaataatttttctataactatttctttttataacaatttggaaaatgtggatgtcttttatttccttgaagcaataaactaagtttctttttataaattttgagtgcaggtgaccaaaaatattgctgaggagtggcacgtttgacatgagtaaaatgtcttaacttcggatttttagcgggaaaatgttataaattggagtttcttttaaatagctttttttaaaatacattaaggatgtctcgctcatgtagaagtcaaattttgttgcaaacgcattgctcccttcacacccaatctctcccctgcaaaaaatcttcacagaattctgtgagaacttttaggtgtgtttttctttgagatacctctggttgccaaacaccaggtaatagattttttaaagttgttattagattattcttacctctcatgatgcatattttagcaatcaccttatcattgtgtctcatgttctgtcctccttatattctttgcccagcaagattctacttatgatgaatgaatgctcttctccttttttcattcaatggtatgaagtatttgttagggttctttagtacttacactttgttgtgtagaaaatgactgtaatgtggtggtcagtgtattcttactgtgattcagagggaatcaaaagtagaaagcaacagcacgtggtcctatcaaagatttggccatctctgcttcactgtcagcctcttaactatatcttcacttactcaatttggttttgtcatgatttttaaatgtagccaatagatcaaggttcttccagtaaacacatatctgcataaatgcctccttgaagtcaataaagaaggaaattgagaagactttaaattaatgataatttagtttttaagtacccacaaataaatttttgaaacattttctttatttgaatacttagatgtcatccaggaaaatcactcaataataattacggcaaatctttaacccctcatttgggtagcttaagataagtaatgccattatgaatcagaattgattcatgactttagttaagaaaatgaaaaggaacatttcacgtatttttaaaaatgatactaaggaataaagaagtacaactattggaaaatatctaagtatatgatttttaaatcctccagtggcattaaatatatgattattagtaattgttagatagggttttattcattcacaaatagaagactagcaagcatgtaactaacaaagtttttacaaaattgactttgtggaatgctccaaatgtttggccattttgaggcacaaggtcaggggtctctttattgatagagctccttctataatttcccagcatacctgcctcacagttatcttcctttcattgttcactctctttttcttctcaatgccatcctgcctaggctcccatcatctgcatctgacacctttcctttctttctttactagtctcctttgcgatgggtgtggctaagctctgtagagccactcagaaactcattgttccattctgtagccagtaaaacatgcctccaaagtgtcacagagtaattctactctctcttttaaattaggtccaccggaaatgttagtgaaaggacattaaaaatgtgacaggtgacatgtttagctaacatggatctggagaaataggaagcagtagaattaaatgtttccctttcaggtttaattgtatttgttcttgggttttgttttatactgagttttaaatatattctccaaataaaaacattattttttctaaccatatgtagagttaatctctttgactaagtaattgaaacaaaagaacatttgttcttttgtgactgcttttttcctaaaacctgagccctcttttttttttttgaaattaaagttgatttcctttttttttttttttttttttttttttttttttttttttttgagacagagtctcgctctgtcgcccaggctggagtgcagtggcgggatctcggctcactgcaagctccgcctcccgggttcacgccattctcctgcctcagcctcccaagtagctgggactacaggcgcccgccactacgcccggctaattttttgtatttttagtagagacggggtttcaccgttttagccgggatggtctcgatctcctgacctcgtgatccgcccgcctcggcctcccaaagtgctgggattacaggcgtgagccaccgcgcccggccttaaaagttgatttccttcttcagtaaggaaacctttttataaatttgttttgcattttaaaagttttactaatcaatgatgaggaaaaagatttgtcttcttgattttaaatagtttcaggatcacaggatgtaatcagatgcttccagtttatttattttcaggtattacactagccatttaatcttttttatttatttattttcttcctgcccctcggatggcatataccagccatttagatactaaactctaatagttaaaccaatagttaaaattgtcctctctaaaacattggctatttaatataccagcttaaatggcctttctctcaagtgagtcactcttagtttaagaaaattatgtgcctttttaaaaaatattatgaaatggtacttcatgacagaaacattttatcagttatagtcttatttgattgaaaattgttgagcatttctgtaaaactttttactttactaaatatttcatctttcctgtgactgttttctcaaagaatttaaaagactcgatgtgtctatgccagaatgtttctcatccttttgaaactgcctgggccaggcgtagtggctcacgctgtaatcccagcacttttggaggccaaggtgggcagatcgcgtgagcccaggagtttgagaccagcctggacaacatggcgaaacggtgtctctacagaaaaatttaaaaattagccaagcatagtggtgcacaactgtagccccagccactcgggaggctgacgtgggaggatcccttgaacctgggggcggaggctgctgtgagccttcatcatgccactgcactccagcctgggcaacaaagcaaaaccctgtctcaaaaaaagaaaagaaaaaaagaaactgcttgaaagtcatgacgaagaatgtcaggaggggacttattctggctgcagttgactttctccttaaatgtcaagtagtgattgatttggataagaagtaaactgttacttttcataacatactttaaggaatttatcaaattctatgtataatgcccattaaaatatactccattctggagtaaagggtaagagtaatatttttaaactagttaataaagtctttagctttcacataaaccatgatatttgaggtgtctaaaatcacagggtcttttttttttttttcagtcttcccagttgttctctgctctattcctaaataaagttaacttgaaaatgca

SEQ ID NO: 3 - DNA, 9905 bp, Homo sapiens
  source 1..9905: mol_type = unassigned DNA
agacgtccgggcagcccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgactgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctattatgctctcaaatacccctaagcatctatactagcctggtatgggtatgaaagatacaaagataaataaaacatagtccctgattctaagaaattcacaatttagcaaaggaaatggactcatagatgctaaccttaaaacaacgtgacaaatgccagacaggacccatcagccaggcactgtgagagcacagagcagggaggttgggtcctgcctgaggagacctggaagggaggcctcacaggaggatgaccaggtctcagtcagcggggaggtggaaagtgcaggtgcatcaggggcaccctgaccgaggaaacagctgccagaggcctccactgctaaagtccacataaggctgaggtcagtcaccctaaacaacctgctccctctaagccaggggatgagcttggagcatcccacaagttccctaaaagttgcagcccccagggggattttgagctatcatctctgcacatgcttagtgagaagactacacaacatttctaagaatctgagattttatattgtcagttaaccactttcattattcattcacctcaggacatgcagaaatatttcagtcagaactgggaaacagaaggacctacattctgctgtcacttatgtgtcaagaagcagatgatcgatgaggcaggtcagttgtaagtgagtcacattgtagcattaaattctagtatttttgtagtttgaaacagtaacttaataaaagagcaaaagctattctagctttcttcttcatattttaattttccaccataaagtttagttgctaaattctattaattttaagattgtgcttcccaaaatagttctcacttcatctgtccagggaggcacagttctgtctggtagaagccgcaaagcccttagcctcttcacggatctggcgactgtgatgggcaggtcaggagaggagctgcccaaagtcccatgattttcacctaacagccctgatcagtcagtactcaaagcttggactccatccctgaaggtcttcctgattgatagcctggccttaataccctacagaaagcctgtccattggctgtttcttcctcagtcagttcctggaagaccttaccccatgaccccagcttcagatgtggtctttggaaacagaggtcgaaggaaagtaaggagctgagagctcacattcataggtgccgccagccttcgtgcatcttcttgcatcatctctaaggagctcctctaattacaccatgcccgtcaccccatgagggatcagagaagggatgagtcttctaaactctatattcgctgtgagtccaggttgtaagggggagcactgtggatgcatcctattgcactccagctgatgacaccaaagcttaggtgtttgctgaaagttcttgatgttgtgacttaccacccctgcctcacaactgcagacataaggggactatggattgcttagcaggaaaggcactggttctcaagggcggctgcccttgggaatcttctggtcccaaccagaaagactgtggcttgattttctcaggtgcagcccagccgtagggccttttcagagcaccccctggttattgcaacattcatcaaagtttctagaacctctggcctaaaggaagggcctggtgggatctacttggcactcgctggggggccaccccccagtgccactctcactaggcctctgattgcacttgtgtaggatgaagctggtgggtgatgggaactcagcacctcccctcaggcagaaaagaatcatctgtggagcttcaaaagaaggggcctggagtctctgcagaccaattcaacccaaatctcgggggctctttcatgattctaatgggcaaccagggttgaaacccttatttctagggtcttcagttgtacaagactgtgggtctgtaccagagcccccgtcagagtagaataaaaggctgggtagggtagagattcccatgtgcagtggagagaacaatctgcagtcactgataagcctgagacttggctcatttcaaaagcgttcaattcatcctcaccagcagttcagctggaaaggggcaaatacccccacctgagctttgaaaacgccctgggaccctctgcattctctaagtaagttatagaaaccagtctcttccctcctttgtgagtgagctgctattccacgtaggcaacacctgttgaaattgccctcaatgtctactctgcatttctttcttgtgataagcacacacttttattgcaacataatgatctgctcacatttccttgcctgggggctgtaaaaccttacagaacagaaatccttgcctctttcaccagccacacctgccataccaggggtacagctttgtactattgaagacacagacaggatttttaaatgtaaatctatttttgtaactttgttgcgggatatagttctctttatgtagcactgaactttgtacaatatatttttagaaactcatttttctactaaaacaaacacagtttactttagagagactgcaatagaatcaaaatttgaaactgaaatctttgtttaaaagggttaagttgaggcaagaggaaagccctttctctctcttataaaaaggcacaacctcattggggagctaagctaggtcattgtcatggtgaagaagagaagcatcgtttttatatttaggaaattttaaaagatgatggaaagcacatttagcttggtctgaggcaggttctgttggggcagtgttaatggaaagggctcactgttgttactactagaaaaatccagttgcatgccatactctcatcatctgccagtgtaaccctgtacatgtaagaaaagcaataacatagcactttgttggtttatatatataatgtgacttcaatgcaaattttatttttatatttacaattgatatgcatttaccagtataaactagacatgtctggagagcctaataatgttcagcacactttggttagttcaccaacagtcttaccaagcctgggcccagccaccctagagaagttattcagccctggctgcagtgacatcacctgaggagcttttaaaagcttgaagcccagctacacctcagaccgattaaacgcaaatctctggggctgaaacccaagcattcgtagtttttaaagctcctgaggtcattccaatgtgcggccaaagttgagaactactggcctagggattagccacaaggacatggacttggaggcaaattctgcaggtgtatgtgattctcaggcctagagagctaagacacaaagacctccacatctgtcgctgagagtcaagaacctgaacagagtttccatgaaggttctccaagcactagaagggagagtgtctaaacaatggttgaaaagcaaaggaaatataaaacagacacctctttccatttcctaaggtttctctctttattaagggtggactagtaataaaatataatattcttgctgcttatgcagctgacattgttgccctccctaaagcaaccaagtagcctttatttcccacagtgaaagaaaacgctggcctatcagttacattacaaaaggcagatttcaagaggattgagtaagtagttggatggctttcataaaaacaagaattcaagaagaggattcatgctttaagaaacatttgttatacattcctcacaaattatacctgggataaaaactatgtagcaggcagtgtgttttccttccatgtctctctgcactacctgcagtgtgtcctctgaggctgcaagtctgtcctatctgaattcccagcagaagcactaagaagctccaccctatcacctagcagataaaactatggggaaaacttaaatctgtgcatacatttctggatgcatttacttatctttaaaaaaaaaggaatcctatgacctgatttggccacaaaaataatcttgctgtacaatacaatctcttggaaattaagagatcctatggatttgatgactggtattagaggtgacaatgtaaccgattaacaacagacagcaataacttcgttttagaaacattcaagcaatagctttatagcttcaacatatggtacgttttaaccttgaaagttttgcaatgatgaaagcagtatttgtacaaatgaaaagcagaattctcttttatatggtttatactgttgatcagaaatgttgattgtgcattgagtattaaaaaattagatgtatattattcattgttctttactcctgagtaccttataataataataatgtattctttgttaacaa

SEQ ID NO: 4 - DNA, 4557 bp, Homo sapiens
  source 1..4557: mol_type = unassigned DNA
cccctccattgggaccggagaaaccaggggagccccccgggcagccgcgcgccccttcccacggggccctttactgcgccgcgcgcccggcccccacccctcgcagcaccccgcgccccgcgccctcccagccgggtccagccggagccatggggccggagccgcagtgagcaccatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcatctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagatgaaataaagacccagggggagaatgggtgttgtatggggaggcaagtgtggggggtccttctccacacccactttgtccatttgcaaatatattttggaaaaca

SEQ ID NO: 5 - DNA, 5911 bp, Homo sapiens
  source 1..5911: mol_type = unassigned DNA
agatgcaggggcgcaaacgccaaaggagaccaggctgtaggaagagaagggcagagcgccggacagctcggcccgctccccgtcctttggggccgcggctggggaactacaaggcccagcaggcagctgcagggggcggaggcggaggagggaccagcgcgggtgggagtgagagagcgagccctcgcgccccgccggcgcatagcgctcggagcgctcttgcggccacaggcgcggcgtcctcggcggcgggcggcagctagcgggagccgggacgccggtgcagccgcagcgcgcggaggaacccgggtgtgccgggagctgggcggccacgtccggacgggaccgagacccctcgtagcgcattgcggcgacctcgccttccccggccgcgagcgcgccgctgcttgaaaagccgcggaacccaaggacttttctccggtccgagctcggggcgccccgcagggcgcacggtacccgtgctgcagtcgggcacgccgcggcgccggggcctccgcagggcgatggagcccggtctgcaaggaaagtgaggcgccgccgctgcgttctggaggaggggggcacaaggtctggagaccccgggtggcggacgggagccctccccccgccccgcctccggggcaccagctccggctccattgttcccgcccgggctggaggcgccgagcaccgagcgccgccgggagtcgagcgccggccgcggagctcttgcgaccccgccaggacccgaacagagcccgggggcggcgggccggagccggggacgcgggcacacgcccgctcgcacaagccacggcggactctcccgaggcggaacctccacgccgagcgagggtcagtttgaaaaggaggatcgagctcactgtggagtatccatggagatgtggagccttgtcaccaacctctaactgcagaactgggatgtggagctggaagtgcctcctcttctgggctgtgctggtcacagccacactctgcaccgctaggccgtccccgaccttgcctgaacaagcccagccctggggagcccctgtggaagtggagtccttcctggtccaccccggtgacctgctgcagcttcgctgtcggctgcgggacgatgtgcagagcatcaactggctgcgggacggggtgcagctggcggaaagcaaccgcacccgcatcacaggggaggaggtggaggtgcaggactccgtgcccgcagactccggcctctatgcttgcgtaaccagcagcccctcgggcagtgacaccacctacttctccgtcaatgtttcagatgctctcccctcctcggaggatgatgatgatgatgatgactcctcttcagaggagaaagaaacagataacaccaaaccaaaccccgtagctccatattggacatccccagaaaagatggaaaagaaattgcatgcagtgccggctgccaagacagtgaagttcaaatgcccttccagtgggaccccaaaccccacactgcgctggttgaaaaatggcaaagaattcaaacctgaccacagaattggaggctacaaggtccgttatgccacctggagcatcataatggactctgtggtgccctctgacaagggcaactacacctgcattgtggagaatgagtacggcagcatcaaccacacataccagctggatgtcgtggagcggtcccctcaccggcccatcctgcaagcagggttgcccgccaacaaaacagtggccctgggtagcaacgtggagttcatgtgtaaggtgtacagtgacccgcagccgcacatccagtggctaaagcacatcgaggtgaatgggagcaagattggcccagacaacctgccttatgtccagatcttgaagactgctggagttaataccaccgacaaagagatggaggtgcttcacttaagaaatgtctcctttgaggacgcaggggagtatacgtgcttggcgggtaactctatcggactctcccatcactctgcatggttgaccgttctggaagccctggaagagaggccggcagtgatgacctcgcccctgtacctggagatcatcatctattgcacaggggccttcctcatctcctgcatggtggggtcggtcatcgtctacaagatgaagagtggtaccaagaagagtgacttccacagccagatggctgtgcacaagctggccaagagcatccctctgcgcagacaggtaacagtgtctgctgactccagtgcatccatgaactctggggttcttctggttcggccatcacggctctcctccagtgggactcccatgctagcaggggtctctgagtatgagcttcccgaagaccctcgctgggagctgcctcgggacagactggtcttaggcaaacccctgggagagggctgctttgggcaggtggtgttggcagaggctatcgggctggacaaggacaaacccaaccgtgtgaccaaagtggctgtgaagatgttgaagtcggacgcaacagagaaagacttgtcagacctgatctcagaaatggagatgatgaagatgatcgggaagcataagaatatcatcaacctgctgggggcctgcacgcaggatggtcccttgtatgtcatcgtggagtatgcctccaagggcaacctgcgggagtacctgcaggcccggaggcccccagggctggaatactgctacaaccccagccacaacccagaggagcagctctcctccaaggacctggtgtcctgcgcctaccaggtggcccgaggcatggagtatctggcctccaagaagtgcatacaccgagacctggcagccaggaatgtcctggtgacagaggacaatgtgatgaagatagcagactttggcctcgcacgggacattcaccacatcgactactataaaaagacaaccaacggccgactgcctgtgaagtggatggcacccgaggcattatttgaccggatctacacccaccagagtgatgtgtggtctttcggggtgctcctgtgggagatcttcactctgggcggctccccataccccggtgtgcctgtggaggaacttttcaagctgctgaaggagggtcaccgcatggacaagcccagtaactgcaccaacgagctgtacatgatgatgcgggactgctggcatgcagtgccctcacagagacccaccttcaagcagctggtggaagacctggaccgcatcgtggccttgacctccaaccaggagtacctggacctgtccatgcccctggaccagtactcccccagctttcccgacacccggagctctacgtgctcctcaggggaggattccgtcttctctcatgagccgctgcccgaggagccctgcctgccccgacacccagcccagcttgccaatggcggactcaaacgccgctgactgccacccacacgccctccccagactccaccgtcagctgtaaccctcacccacagcccctgctgggcccaccacctgtccgtccctgtcccctttcctgctggcaggagccggctgcctaccaggggccttcctgtgtggcctgccttcaccccactcagctcacctctccctccacctcctctccacctgctggtgagaggtgcaaagaggcagatctttgctgccagccacttcatcccctcccagatgttggaccaacacccctccctgccaccaggcactgcctggagggcagggagtgggagccaatgaacaggcatgcaagtgagagcttcctgagctttctcctgtcggtttggtctgttttgccttcacccataagcccctcgcactctggtggcaggtgccttgtcctcagggctacagcagtagggaggtcagtgcttcgtgcctcgattgaaggtgacctctgccccagataggtggtgccagtggcttattaattccgatactagtttgctttgctgaccaaatgcctggtaccagaggatggtgaggcgaaggccaggttgggggcagtgttgtggccctggggcccagccccaaactgggggctctgtatatagctatgaagaaaacacaaagtgtataaatctgagtatatatttacatgtctttttaaaagggtcgttaccagagatttacccatcgggtaagatgctcctggtggctgggaggcatcagttgctatatattaaaaacaaaaaagaaaaaaaaggaaaatgtttttaaaaaggtcatatattttttgctacttttgctgttttatttttttaaattatgttctaaacctattttcagtttaggtccctcaataaaaattgctgctgcttcatttatctatgggctgtatgaaaagggtgggaatgtccactggaaagaagggacacccacgggccctggggctaggtctgtcccgagggcaccgcatgctcccggcgcaggttccttgtaacctcttcttcctaggtcctgcacccagacctcacgacgcacctcctgcctctccgctgcttttggaaagtcagaaaaagaagatgtctgcttcgagggcaggaaccccatccatgcagtagaggcgctgggcagagagtcaaggcccagcagccatcgaccatggatggtttcctccaaggaaaccggtggggttgggctggggagggggcacctacctaggaatagccacggggtagagctacagtgattaagaggaaagcaagggcgcggttgctcacgcctgtaatcccagcactttgggacaccgaggtgggcagatcacttcaggtcaggagtttgagaccagcctggccaacttagtgaaaccccatctctactaaaaatgcaaaaattatccaggcatggtggcacacgcctgtaatcccagctccacaggaggctgaggcagaatcccttgaagctgggaggcggaggttgcagtgagccgagattgcgccattgcactccagcctgggcaacagagaaaacaaaaaggaaaacaaatgatgaaggtctgcagaaactgaaacccagacatgtgtctgccccctctatgtgggcatggttttgccagtgcttctaagtgcaggagaacatgtcacctgaggctagttttgcattcaggtccctggcttcgtttcttgttggtatgcctccccagatcgtccttcctgtatccatgtgaccagactgtatttgttgggactgtcgcagatcttggcttcttacagttcttcctgtccaaactccatcctgtccctcaggaacggggggaaaattctccgaatgtttttggttttttggctgcttggaatttacttctgccacctgctggtcatcactgtcctcactaagtggattctggctcccccgtacctcatggctcaaactaccactcctcagtcgctatattaaagcttatattttgctggattactgctaaatacaaaagaaagttcaatatgttttcatttctgtagggaaaatgggattgctgctttaaatttctgagctagggattttttggcagctgcagtgttggcgactattgtaaaattctctttgtttctctctgtaaatagcacctgctaacattacaatttgtatttatgtttaaagaaggcatcatttggtgaacagaactaggaaatgaatttttagctcttaaaagcatttgctttgagaccgcacaggagtgtctttccttgtaaaacagtgatgataatttctgccttggccctaccttgaagcaatgttgtgtgaagggatgaagaatctaaaagtcttcataagtccttgggagaggtgctagaaaaatataaggcactatcataattacagtgatgtccttgctgttactactcaaatcacccacaaatttccccaaagactgcgctagctgtcaaataaaagacagtgaaattgacctgaaaaaaaaaaaaaaaaa

SEQ ID NO: 6 - DNA, 4624 bp, Homo sapiens
  source 1..4624: mol_type = unassigned DNA
gagagcgcggtggagagccgagcgggcgggcggcgggtgcggagcgggcgagggagcgcgcgcggccgccacaaagctcgggcgccgcggggctgcatgcggcgtacctggcccggcgcggcgactgctctccgggctggcgggggccggccgcgagccccgggggccccgaggccgcagcttgcctgcgcgctctgagccttcgcaactcgcgagcaaagtttggtggaggcaacgccaagcctgagtcctttcttcctctcgttccccaaatccgagggcagcccgcgggcgtcatgcccgcgctcctccgcagcctggggtacgcgtgaagcccgggaggcttggcgccggcgaagacccaaggaccactcttctgcgtttggagttgctccccgcaaccccgggctcgtcgctttctccatcccgacccacgcggggcgcggggacaacacaggtcgcggaggagcgttgccattcaagtgactgcagcagcagcggcagcgcctcggttcctgagcccaccgcaggctgaaggcattgcgcgtagtccatgcccgtagaggaagtgtgcagatgggattaacgtccacatggagatatggaagaggaccggggattggtaccgtaaccatggtcagctggggtcgtttcatctgcctggtcgtggtcaccatggcaaccttgtccctggcccggccctccttcagtttagttgaggataccacattagagccagaagagccaccaaccaaataccaaatctctcaaccagaagtgtacgtggctgcgccaggggagtcgctagaggtgcgctgcctgttgaaagatgccgccgtgatcagttggactaaggatggggtgcacttggggcccaacaataggacagtgcttattggggagtacttgcagataaagggcgccacgcctagagactccggcctctatgcttgtactgccagtaggactgtagacagtgaaacttggtacttcatggtgaatgtcacagatgccatctcatccggagatgatgaggatgacaccgatggtgcggaagattttgtcagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtagtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggagatagccatttactgcataggggtcttcttaatcgcctgtatggtggtaacagtcatcctgtgccgaatgaagaacacgaccaagaagccagacttcagcagccagccggctgtgcacaagctgaccaaacgtatccccctgcggagacaggtaacagtttcggctgagtccagctcctccatgaactccaacaccccgctggtgaggataacaacacgcctctcttcaacggcagacacccccatgctggcaggggtctccgagtatgaacttccagaggacccaaaatgggagtttccaagagataagctgacactgggcaagcccctgggagaaggttgctttgggcaagtggtcatggcggaagcagtgggaattgacaaagacaagcccaaggaggcggtcaccgtggccgtgaagatgttgaaagatgatgccacagagaaagacctttctgatctggtgtcagagatggagatgatgaagatgattgggaaacacaagaatatcataaatcttcttggagcctgcacacaggatgggcctctctatgtcatagttgagtatgcctctaaaggcaacctccgagaatacctccgagcccggaggccacccgggatggagtactcctatgacattaaccgtgttcctgaggagcagatgaccttcaaggacttggtgtcatgcacctaccagctggccagaggcatggagtacttggcttcccaaaaatgtattcatcgagatttagcagccagaaatgttttggtaacagaaaacaatgtgatgaaaatagcagactttggactcgccagagatatcaacaatatagactattacaaaaagaccaccaatgggcggcttccagtcaagtggatggctccagaagccctgtttgatagagtatacactcatcagagtgatgtctggtccttcggggtgttaatgtgggagatcttcactttagggggctcgccctacccagggattcccgtggaggaactttttaagctgctgaaggaaggacacagaatggataagccagccaactgcaccaacgaactgtacatgatgatgagggactgttggcatgcagtgccctcccagagaccaacgttcaagcagttggtagaagacttggatcgaattctcactctcacaaccaatgaggaatacttggacctcagccaacctctcgaacagtattcacctagttaccctgacacaagaagttcttgttcttcaggagatgattctgttttttctccagaccccatgccttacgaaccatgccttcctcagtatccacacataaacggcagtgttaaaacatgaatgactgtgtctgcctgtccccaaacaggacagcactgggaacctagctacactgagcagggagaccatgcctcccagagcttgttgtctccacttgtatatatggatcagaggagtaaataattggaaaagtaatcagcatatgtgtaaagatttatacagttgaaaacttgtaatcttccccaggaggagaagaaggtttctggagcagtggactgccacaagccaccatgtaacccctctcacctgccgtgcgtactggctgtggaccagtaggactcaaggtggacgtgcgttctgccttccttgttaattttgtaataattggagaagatttatgtcagcacacacttacagagcacaaatgcagtatataggtgctggatgtatgtaaatatattcaaattatgtataaatatatattatatatttacaaggagttattttttgtattgattttaaatggatgtcccaatgcacctagaaaattggtctctctttttttaatagctatttgctaaatgctgttcttacacataatttcttaattttcaccgagcagaggtggaaaaatacttttgctttcagggaaaatggtataacgttaatttattaataaattggtaatatacaaaacaattaatcatttatagttttttttgtaatttaagtggcatttctatgcaggcagcacagcagactagttaatctattgcttggacttaactagttatcagatcctttgaaaagagaatatttacaatatatgactaatttggggaaaatgaagttttgatttatttgtgtttaaatgctgctgtcagacgattgttcttagacctcctaaatgccccatattaaaagaactcattcataggaaggtgtttcattttggtgtgcaaccctgtcattacgtcaacgcaacgtctaactggacttcccaagataaatggtaccagcgtcctcttaaaagatgccttaatccattccttgaggacagaccttagttgaaatgatagcagaatgtgcttctctctggcagctggccttctgcttctgagttgcacattaatcagattagcctgtattctcttcagtgaattttgataatggcttccagactctttggcgttggagacgcctgttaggatcttcaagtcccatcatagaaaattgaaacacagagttgttctgctgatagttttggggatacgtccatctttttaagggattgctttcatctaattctggcaggacctcaccaaaagatccagcctcatacctacatcagacaaaatatcgccgttgttccttctgtactaaagtattgtgttttgctttggaaacacccactcactttgcaatagccgtgcaagatgaatgcagattacactgatcttatgtgttacaaaattggagaaagtatttaataaaacctgttaatttttatactgacaataaaaatgtttctacagatattaatgttaacaagacaaaataaatgtcacgcaacttatttttttaa

SEQ ID NO: 7 - DNA, 4301 bp, Homo sapiens
  source 1..4301: mol_type = unassigned DNA
agtgcgcggtggcggcggcgtcgcgggcagctggcgccgcgcggtcctgctctgccggtcgcacggacgcaccggcgggccgccggccggagggacggggcgggagctgggcccgcggacagcgagccggagcgggagccgcgcgtagcgagccgggctccggcgctcgccagtctcccgagcggcgcccgcctcccgccggtgcccgcgccgggccgtggggggcagcatgcccgcgcgcgctgcctgaggacgccgcggcccccgcccccgccatgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacgaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacgagtacctggacctgtcggcgcctttcgagcagtactccccgggtggccaggacacccccagctccagctcctcaggggacgactccgtgtttgcccacgacctgctgcccccggccccacccagcagtgggggctcgcggacgtgaagggccactggtccccaacaatgtgaggggtccctagcagcccaccctgctgctggtgcacagccactccccggcatgagactcagtgcagatggagagacagctacacagagctttggtctgtgtgtgtgtgtgtgcgtgtgtgtgtgtgtgtgtgcacatccgcgtgtgcctgtgtgcgtgcgcatcttgcctccaggtgcagaggtaccctgggtgtccccgctgctgtgcaacggtctcctgactggtgctgcagcaccgaggggcctttgttctggggggacccagtgcagaatgtaagtgggcccacccggtgggacccccgtggggcagggagctgggcccgacatggctccggcctctgcctttgcaccacgggacatcacagggtgggcctcggcccctcccacacccaaagctgagcctgcagggaagccccacatgtccagcaccttgtgcctggggtgttagtggcaccgcctccccacctccaggctttcccacttcccaccctgcccctcagagactgaaattacgggtacctgaagatgggagcctttaccttttatgcaaaaggtttattccggaaactagtgtacatttctataaatagatgctgtgtatatggtatatatacatatatatatataacatatatggaagaggaaaaggctggtacaacggaggcctgcgaccctgggggcacaggaggcaggcatggccctgggcggggcgtgggggggcgtggagggaggccccagggggtctcacccatgcaagcagaggaccagggccttttctggcaccgcagttttgttttaaaactggacctgtatatttgtaaagctatttatgggcccctggcactcttgttcccacaccccaacacttccagcatttagctggccacatggcggagagttttaatttttaacttattgacaaccgagaaggtttatcccgccgatagagggacggccaagaatgtacgtccagcctgccccggagctggaggatcccctccaagcctaaaaggttgttaatagttggaggtgattccagtgaagatattttatttcctttgtcctttttcaggagaattagatttctataggatttttctttaggagatttattttttggacttcaaagcaagctggtattttcatacaaattcttctaattgctgtgtgtcccaggcagggagacggtttccagggaggggccggccctgtgtgcaggttccgatgttattagatgttacaagtttatatatatctatatatataatttattgagtttttacaagatgtatttgttgtagacttaacacttcttacgcaatgcttctagagttttatagcctggactgctacctttcaaagcttggagggaagccgtgaattcagttggttcgttctgtactgttactgggccctgagtctgggcagctgtcccttgcttgcctgcagggccatggctcagggtggtctcttcttggggcccagtgcatggtggccagaggtgtcacccaaaccggcaggtgcgattttgttaacccagcgacgaactttccgaaaaataaagacacctggttgctaa

SEQ ID NO: 8 - DNA, 6822 bp, Homo sapiens
  source 1..6822: mol_type = unassigned DNA
agacacgtgctggggcgggcaggcgagcgcctcagtctggtcgcctggcggtgcctccggccccaacgcgcccgggccgccgcgggccgcgcgcgccgatgcccggctgagtcactggcagggcagcgcgcgtgtgggaaggggcggagggagtgcggccggcgggcgggcggggcgctgggctcagcccggccgcaggtgacccggaggccctcgccgcccgcggcgccccgagcgctttgtgagcagatgcggagccgagtggagggcgcgagccagatgcggggcgacagctgacttgctgagaggaggcggggaggcgcggagcgcgcgtgtggtccttgcgccgctgacttctccactggttcctgggcaccgaaagataaacctctcataatgaaggcccccgctgtgcttgcacctggcatcctcgtgctcctgtttaccttggtgcagaggagcaatggggagtgtaaagaggcactagcaaagtccgagatgaatgtgaatatgaagtatcagcttcccaacttcaccgcggaaacacccatccagaatgtcattctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggctacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggttttcccaaatagtgcaccccttgaaggagggacaaggctgaccatatgtggctgggactttggatttcggaggaataataaatttgatttaaagaaaactagagttctccttggaaatgagagctgcaccttgactttaagtgagagcacgatgaatacattgaaatgcacagttggtcctgccatgaataagcatttcaatatgtccataattatttcaaatggccacgggacaacacaatacagtacattctcctatgtggatcctgtaataacaagtatttcgccgaaatacggtcctatggctggtggcactttacttactttaactggaaattacctaaacagtgggaattctagacacatttcaattggtggaaaaacatgtactttaaaaagtgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattagtggtgggagcacaataacaggtgttgggaaaaacctgaattcagttagtgtcccgagaatggtcataaatgtgcatgaagcaggaaggaactttacagtggcatgtcaacatcgctctaattcagagataatctgttgtaccactccttccctgcaacagctgaatctgcaactccccctgaaaaccaaagcctttttcatgttagatgggatcctttccaaatactttgatctcatttatgtacataatcctgtgtttaagccttttgaaaagccagtgatgatctcaatgggcaatgaaaatgtactggaaattaagggaaatgatattgaccctgaagcagttaaaggtgaagtgttaaaagttggaaataagagctgtgagaatatacacttacattctgaagccgttttatgcacggtccccaatgacctgctgaaattgaacagcgagctaaatatagagtggaagcaagcaatttcttcaaccgtccttggaaaagtaatagttcaaccagatcagaatttcacaggattgattgctggtgttgtctcaatatcaacagcactgttattactacttgggtttttcctgtggctgaaaaagagaaagcaaattaaagatctgggcagtgaattagttcgctacgatgcaagagtacacactcctcatttggataggcttgtaagtgcccgaagtgtaagcccaactacagaaatggtttcaaatgaatctgtagactaccgagctacttttccagaagatcagtttcctaattcatctcagaacggttcatgccgacaagtgcagtatcctctgacagacatgtcccccatcctaactagtggggactctgatatatccagtccattactgcaaaatactgtccacattgacctcagtgctctaaatccagagctggtccaggcagtgcagcatgtagtgattgggcccagtagcctgattgtgcatttcaatgaagtcataggaagagggcattttggttgtgtatatcatgggactttgttggacaatgatggcaagaaaattcactgtgctgtgaaatccttgaacagaatcactgacataggagaagtttcccaatttctgaccgagggaatcatcatgaaagattttagtcatcccaatgtcctctcgctcctgggaatctgcctgcgaagtgaagggtctccgctggtggtcctaccatacatgaaacatggagatcttcgaaatttcattcgaaatgagactcataatccaactgtaaaagatcttattggctttggtcttcaagtagccaaaggcatgaaatatcttgcaagcaaaaagtttgtccacagagacttggctgcaagaaactgtatgctggatgaaaaattcacagtcaaggttgctgattttggtcttgccagagacatgtatgataaagaatactatagtgtacacaacaaaacaggtgcaaagctgccagtgaagtggatggctttggaaagtctgcaaactcaaaagtttaccaccaagtcagatgtgtggtcctttggcgtgctcctctgggagctgatgacaagaggagccccaccttatcctgacgtaaacacctttgatataactgtttacttgttgcaagggagaagactcctacaacccgaatactgcccagaccccttatatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgtcatcagaagataacgctgatgatgaggtggacacacgaccagcctccttctgggagacatcatagtgctagtactatgtcaaagcaacagtccacactttgtccaatggttttttcactgcctgacctttaaaaggccatcgatattctttgctcttgccaaaattgcactattataggacttgtattgttatttaaattactggattctaaggaatttcttatctgacagagcatcagaaccagaggcttggtcccacaggccacggaccaatggcctgcagccgtgacaacactcctgtcatattggagtccaaaacttgaattctgggttgaattttttaaaaatcaggtaccacttgatttcatatgggaaattgaagcaggaaatattgagggcttcttgatcacagaaaactcagaagagatagtaatgctcaggacaggagcggcagccccagaacaggccactcatttagaattctagtgtttcaaaacacttttgtgtgttgtatggtcaataacatttttcattactgatggtgtcattcacccattaggtaaacattcccttttaaatgtttgtttgttttttgagacaggatctcactctgttgccagggctgtagtgcagtggtgtgatcatagctcactgcaacctccacctcccaggctcaagcctcccgaatagctgggactacaggcgcacaccaccatccccggctaatttttgtattttttgtagagacggggttttgccatgttgccaaggctggtttcaaactcctggactcaagaaatccacccacctcagcctcccaaagtgctaggattacaggcatgagccactgcgcccagcccttataaatttttgtatagacattcctttggttggaagaatatttataggcaatacagtcaaagtttcaaaatagcatcacacaaaacatgtttataaatgaacaggatgtaatgtacatagatgacattaagaaaatttgtatgaaataatttagtcatcatgaaatatttagttgtcatataaaaacccactgtttgagaatgatgctactctgatctaatgaatgtgaacatgtagatgttttgtgtgtatttttttaaatgaaaactcaaaataagacaagtaatttgttgataaatatttttaaagataactcagcatgtttgtaaagcaggatacattttactaaaaggttcattggttccaatcacagctcataggtagagcaaagaaagggtggatggattgaaaagattagcctctgtctcggtggcaggttcccacctcgcaagcaattggaaacaaaacttttggggagttttattttgcattagggtgtgttttatgttaagcaaaacatactttagaaacaaatgaaaaaggcaattgaaaatcccagctatttcacctagatggaatagccaccctgagcagaactttgtgatgcttcattctgtggaattttgtgcttgctactgtatagtgcatgtggtgtaggttactctaactggttttgtcgacgtaaacatttaaagtgttatattttttataaaaatgtttatttttaatgatatgagaaaaattttgttaggccacaaaaacactgcactgtgaacattttagaaaaggtatgtcagactgggattaatgacagcatgattttcaatgactgtaaattgcgataaggaaatgtactgattgccaatacaccccaccctcattacatcatcaggacttgaagccaagggttaacccagcaagctacaaagagggtgtgtcacactgaaactcaatagttgagtttggctgttgttgcaggaaaatgattataactaaaagctctctgatagtgcagagacttaccagaagacacaaggaattgtactgaagagctattacaatccaaatattgccgtttcataaatgtaataagtaatactaattcacagagtattgtaaatggtggatgacaaaagaaaatctgctctgtggaaagaaagaactgtctctaccagggtcaagagcatgaacgcatcaatagaaagaactcggggaaacatcccatcaacaggactacacacttgtatatacattcttgagaacactgcaatgtgaaaatcacgtttgctatttataaacttgtccttagattaatgtgtctggacagattgtgggagtaagtgattcttctaagaattagatacttgtcactgcctatacctgcagctgaactgaatggtacttcgtatgttaatagttgttctgataaatcatgcaattaaagtaaagtgatgcaa

SEQ ID NO: 9 - DNA, 3191 bp, Homo sapiens
  source 1..3191: mol_type = unassigned DNA
aatcgcgggcgcttgggccgccatcttagatggcgggagtaagaggaaaacgattgtgaggcgggaacggctttctgctgccttttttgggccccgaaaagggtcagctggccgggctttggggcgcgtgccctgaggcgcggagcgcgtttgctacgatgcgggggctgctcggggctccgtcccctgggctggggacgcgccgaatgtgaccgcctcccgctccctcacccgccgcggggaggaggagcgggcgagaagctgccgccgaacgacaggacgttggggcggcctggctccctcaggtttaagaattgtttaagctgcatcaatggagcacatacagggagcttggaagacgatcagcaatggttttggattcaaagatgccgtgtttgatggctccagctgcatctctcctacaatagttcagcagtttggctatcagcgccgggcatcagatgatggcaaactcacagatccttctaagacaagcaacactatccgtgttttcttgccgaacaagcaaagaacagtggtcaatgtgcgaaatggaatgagcttgcatgactgccttatgaaagcactcaaggtgaggggcctgcaaccagagtgctgtgcagtgttcagacttctccacgaacacaaaggtaaaaaagcacgcttagattggaatactgatgctgcgtctttgattggagaagaacttcaagtagatttcctggatcatgttcccctcacaacacacaactttgctcggaagacgttcctgaagcttgccttctgtgacatctgtcagaaattcctgctcaatggatttcgatgtcagacttgtggctacaaatttcatgagcactgtagcaccaaagtacctactatgtgtgtggactggagtaacatcagacaactcttattgtttccaaattccactattggtgatagtggagtcccagcactaccttctttgactatgcgtcgtatgcgagagtctgtttccaggatgcctgttagttctcagcacagatattctacacctcacgccttcacctttaacacctccagtccctcatctgaaggttccctctcccagaggcagaggtcgacatccacacctaatgtccacatggtcagcaccaccctgcctgtggacagcaggatgattgaggatgcaattcgaagtcacagcgaatcagcctcaccttcagccctgtccagtagccccaacaatctgagcccaacaggctggtcacagccgaaaacccccgtgccagcacaaagagagcgggcaccagtatctgggacccaggagaaaaacaaaattaggcctcgtggacagagagattcaagctattattgggaaatagaagccagtgaagtgatgctgtccactcggattgggtcaggctcttttggaactgtttataagggtaaatggcacggagatgttgcagtaaagatcctaaaggttgtcgacccaaccccagagcaattccaggccttcaggaatgaggtggctgttctgcgcaaaacacggcatgtgaacattctgcttttcatggggtacatgacaaaggacaacctggcaattgtgacccagtggtgcgagggcagcagcctctacaaacacctgcatgtccaggagaccaagtttcagatgttccagctaattgacattgcccggcagacggctcagggaatggactatttgcatgcaaagaacatcatccatagagacatgaaatccaacaatatatttctccatgaaggcttaacagtgaaaattggagattttggtttggcaacagtaaagtcacgctggagtggttctcagcaggttgaacaacctactggctctgtcctctggatggccccagaggtgatccgaatgcaggataacaacccattcagtttccagtcggatgtctactcctatggcatcgtattgtatgaactgatgacgggggagcttccttattctcacatcaacaaccgagatcagatcatcttcatggtgggccgaggatatgcctccccagatcttagtaagctatataagaactgccccaaagcaatgaagaggctggtagctgactgtgtgaagaaagtaaaggaagagaggcctctttttccccagatcctgtcttccattgagctgctccaacactctctaccgaagatcaaccggagcgcttccgagccatccttgcatcgggcagcccacactgaggatatcaatgcttgcacgctgaccacgtccccgaggctgcctgtcttctagttgactttgcacctgtcttcaggctgccaggggaggaggagaagccagcaggcaccacttttctgctccctttctccagaggcagaacacatgttttcagagaagctgctgctaaggaccttctagactgctcacagggccttaacttcatgttgccttcttttctatccctttgggccctgggagaaggaagccatttgcagtgctggtgtgtcctgctccctccccacattccccatgctcaaggcccagccttctgtagatgcgcaagtggatgttgatggtagtacaaaaagcaggggcccagccccagctgttggctacatgagtatttagaggaagtaaggtagcaggcagtccagccctgatgtggagacacatgggattttggaaatcagcttctggaggaatgcatgtcacaggcgggactttcttcagagagtggtgcagcgccagacattttgcacataaggcaccaaacagcccaggactgccgagactctggccgcccgaaggagcctgctttggtactatggaacttttcttaggggacacgtcctcctttcacagcttctaaggtgtccagtgcattgggatggttttccaggcaaggcactcggccaatccgcatctcagccctctcagggagcagtcttccatcatgctgaattttgtcttccaggagctgcccctatggggcggggccgcagggccagccttgtttctctaacaaacaaacaaacaaacagccttgtttctctagtcacatcatgtgtatacaaggaagccaggaatacaggttttcttgatgatttgggttttaattttgtttttattgcacctgacaaaatacagttatctgatggtccctcaattatgttattttaataaaataaattaaatttaggtgtaa

SEQ ID NO: 10 - DNA, 4174 bp, Homo sapiens
  source 1..4174: mol_type = unassigned DNA
agtcccgcgaccgaagcagggcgcgcagcagcgctgagtgccccggaacgtgcgtcgcgcccccagtgtccgtcgcgtccgccgcgccccgggcggggatggggcggccagactgagcgccgcacccgccatccagacccgccggccctagccgcagtccctccagccgtggccccagcgcgcacgggcgatggcgaaggcgacgtccggtgccgcggggctgcgtctgctgttgctgctgctgctgccgctgctaggcaaagtggcattgggcctctacttctcgagggatgcttactgggagaagctgtatgtggaccaggcggccggcacgcccttgctgtacgtccatgccctgcgggacgcccctgaggaggtgcccagcttccgcctgggccagcatctctacggcacgtaccgcacacggctgcatgagaacaactggatctgcatccaggaggacaccggcctcctctaccttaaccggagcctggaccatagctcctgggagaagctcagtgtccgcaaccgcggctttcccctgctcaccgtctacctcaaggtcttcctgtcacccacatcccttcgtgagggcgagtgccagtggccaggctgtgcccgcgtatacttctccttcttcaacacctcctttccagcctgcagctccctcaagccccgggagctctgcttcccagagacaaggccctccttccgcattcgggagaaccgacccccaggcaccttccaccagttccgcctgctgcctgtgcagttcttgtgccccaacatcagcgtggcctacaggctcctggagggtgagggtctgcccttccgctgcgccccggacagcctggaggtgagcacgcgctgggccctggaccgcgagcagcgggagaagtacgagctggtggccgtgtgcaccgtgcacgccggcgcgcgcgaggaggtggtgatggtgcccttcccggtgaccgtgtacgacgaggacgactcggcgcccaccttccccgcgggcgtcgacaccgccagcgccgtggtggagttcaagcggaaggaggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactataggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccagatcgggaaagtctgtgtggaaaactgccaggcattcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagcttcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctagcaccgctgtcccctctgcactatccttcctctctgtgatgctttttaaaaatgtttctggtctgaacaaaaccaaagtctgctctgaacctttttatttgtaaatgtctgactttgcatccagtttacatttaggcattattgcaactatgtttttctaaaaggaagtgaaaataagtgtaattaccacattgcccagcaacttaggatggtagaggaaaaaacagatcagggcggaactctcaggggagaccaagaacaggttgaataaggcgcttctggggtgggaatcaagtcatagtacttctactttaactaagtggataaatatacaaatctggggaggtattcagttgagaaaggagccaccagcaccactcagcctgcactgggagcacagccaggttcccccagacccctcctgggcaggcaggtgcctctcagaggccacccggcactggcgagcagccactggccaagcctcagccccagtcccagccacatgtcctccatcaggggtagcgaggttgcaggagctggctggccctgggaggacgcacccccactgctgttttcacatcctttcccttacccaccttcaggacggttgtcacttatgaagtcagtgctaaagctggagcagttgctttttgaaagaacatggtctgtggtgctgtggtcttacaatggacagtaaatatggttcttgccaaaactccttcttttgtctttgattaaatactagaaatttaaaaaaaaaaaaaaa

SEQ ID NO: 11 - DNA, 8469 bp, Homo sapiens
  source 1..8469: mol_type = unassigned DNA
gcacttctaagaactaacctttagtcactgggtgactttatgggagtaaaaggaagctgttatgaaatagctcttatggaactgttacaagctttcaagcattcaaaggtctaaatgaaaaaggctaagtattatttcaaaaggcaagtatatcctaatatagcaaaacaaacaaagcaaaatccatcagctactcctccaattgaagtgatgaagcccaaataattcatatagcaaaatggagaaaattagaccggccatctaaaaatctgccattggtgaagtgatgaagaacatttactgtcttattccgaagcttgtcaattttgcaactcttggctgcctatggatttctgtggtgcagtgtacagttttaaatagctgcctaaagtcgtgtgtaactaatctgggccagcagcttgaccttggcacaccacataatctgagtgaaccgtgtatccaaggatgtcacttttggaactctgtagatcagaaaaactgtgctttaaagtgtcgggagtcgtgtgaggttggctgtagcagcgcggaaggtgcatatgaagaggaagtactggaaaatgcagacctaccaactgctccctttgcttcttccattggaagccacaatatgacattacgatggaaatctgcaaacttctctggagtaaaatacatcattcagtggaaatatgcacaacttctgggaagctggacttatactaagactgtgtccagaccgtcctatgtggtcaagcccctgcaccccttcactgagtacattttccgagtggtttggatcttcacagcgcagctgcagctctactcccctccaagtcccagttacaggactcatcctcatggagttcctgaaactgcacctttgattaggaatattgagagctcaagtcccgacactgtggaagtcagctgggatccacctcaattcccaggtggacctattttgggttataacttaaggctgatcagcaaaaatcaaaaattagatgcagggacacagagaaccagtttccagttttactccactttaccaaatactatctacaggttttctattgcagcagtaaatgaagttggtgagggtccagaagcagaatctagtattaccacttcatcttcagcagttcaacaagaggaacagtggctctttttatccagaaaaacttctctaagaaagagatctttaaaacatttagtagatgaagcacattgccttcggttggatgctatataccataatattacaggaatatctgttgatgtccaccagcaaattgtttatttctctgaaggaactctcatatgggcgaagaaggctgccaacatgtctgatgtatctgacctgagaattttttacagaggttcaggattaatttcttctatctccatagattggctttatcaaagaatgtatttcatcatggatgaactggtatgtgtctgtgatttagagaactgctcaaacatcgaggaaattactccaccctctattagtgcacctcaaaaaattgtggctgattcatacaatgggtatgtcttttacctcctgagagatggcatttatagagcagaccttcctgtaccatctggccggtgtgcagaagctgtgcgtattgtggagagttgcacgttaaaggactttgcaatcaagccacaagccaagcgaatcatttacttcaatgacactgcccaagtcttcatgtcaacatttctggatggctctgcttcccatctcatcctacctcgcatcccctttgctgatgtgaaaagttttgcttgtgaaaacaatgactttcttgtcacagatggcaaggtcattttccaacaggatgctttgtcttttaatgaattcatcgtgggatgtgacctgagtcacatagaagaatttgggtttggtaacttggtcatctttggctcatcctcccagctgcaccctctgccaggccgcccgcaggagctttcggtgctgtttggctctcaccaggctcttgttcaatggaagcctcctgcccttgccataggagccaatgtcatcctgatcagtgatattattgaactctttgaattaggcccttctgcctggcagaactggacctatgaggtgaaagtatccacccaagaccctcctgaagtcactcatattttcttgaacataagtggaaccatgctgaatgtacctgagctgcagagtgctatgaaatacaaggtttctgtgagagcaagttctccaaagaggccaggcccctggtcagagccctcagtgggtactaccctggtgccagctagtgaaccaccatttatcatggctgtgaaagaagatgggctttggagtaaaccattaaatagctttggcccaggagagttcttatcctctgatataggaaatgtgtcagacatggattggtataacaacagcctctactacagtgacacgaaaggcgacgtttttgtgtggctgctgaatgggacggatatctcagagaattatcacctacccagcattgcaggagcaggggctttagcttttgagtggctgggtcactttctctactgggctggaaagacatatgtgatacaaaggcagtctgtgttgacgggacacacagacattgttacccacgtgaagctattggtgaatgacatggtggtggattcagttggtggatatctctactggaccacactctattcagtggaaagcaccagactaaatggggaaagttcccttgtactacagacacagccttggttttctgggaaaaaggtaattgctctaactttagacctcagtgatgggctcctgtattggttggttcaagacagtcaatgtattcacctgtacacagctgttcttcggggacagagcactggggataccaccatcacagaatttgcagcctggagtacttctgaaatttcccagaatgcactgatgtactatagtggtcggctgttctggatcaatggctttaggattatcacaactcaagaaataggtcagaaaaccagtgtctctgttttggaaccagccagatttaatcagttcacaattattcagacatcccttaagcccctgccagggaacttttcctttacccctaaggttattccagattctgttcaagagtcttcatttaggattgaaggaaatgcttcaagttttcaaatcctgtggaatggtccccctgcggtagactggggtgtagttttctacagtgtagaatttagtgctcattctaagttcttggctagtgaacaacactctttacctgtatttactgtggaaggactggaaccttatgccttatttaatctttctgtcactccttatacctactggggaaagggccccaaaacatctctgtcacttcgagcacctgaaacagttccatcagcaccagagaaccccagaatatttatattaccaagtggaaaatgctgcaacaagaatgaagttgtggtggaatttaggtggaacaaacctaagcatgaaaatggggtgttaacaaaatttgaaattttctacaatatatccaatcaaagtattacaaacaaaacatgtgaagactggattgctgtcaatgtcactccctcagtgatgtcttttcaacttgaaggcatgagtcccagatgctttattgccttccaggttagggcctttacatctaaggggccaggaccatatgctgacgttgtaaagtctacaacatcagaaatcaacccatttcctcacctcataactcttcttggtaacaagatagtttttttagatatggatcaaaatcaagttgtgtggacgttttcagcagaaagagttatcagtgccgtttgctacacagctgataatgagatgggatattatgctgaaggggactcactctttcttctgcacttgcacaatcgctctagctctgagcttttccaagattcactggtttttgatatcacagttattacaattgactggatttcaaggcacctctactttgcactgaaagaatcacaaaatggaatgcaagtatttgatgttgatcttgaacacaaggtgaaatatcccagagaggtgaagattcacaataggaattcaacaataatttctttttctgtatatcctcttttaagtcgcttgtattggacagaagtttccaattttggctaccagatgttctactacagtattatcagtcacaccttgcaccgaattctgcaacccacagctacaaaccaacaaaacaaaaggaatcaatgttcttgtaatgtgactgaatttgagttaagtggagcaatggctattgatacctctaacctagagaaaccattgatatactttgccaaagcacaagagatctgggcaatggatctggaaggctgtcagtgttggagagttatcacagtacctgctatgctcgcaggaaaaacccttgttagcttaactgtggatggagatcttatatactggatcatcacagcaaaggacagcacacagatttatcaggcaaagaaaggaaatggggccatcgtttcccaggtgaaggccctaaggagtaggcatatcttggcttacagttcagttatgcagccttttccagataaagcgtttctgtctctagcttcagacactgtggaaccaactatacttaatgccactaacactagcctcacaatcagattacctctggccaagacaaacctcacatggtatggcatcaccagccctactccaacatacctggtttattatgcagaagttaatgacaggaaaaacagctctgacttgaaatatagaattctggaatttcaggacagtatagctcttattgaagatttacaaccattttcaacatacatgatacagatagctgtaaaaaattattattcagatcctttggaacatttaccaccaggaaaagagatttggggaaaaactaaaaatggagtaccagaggcagtgcagctcattaatacaactgtgcggtcagacaccagcctcattatatcttggagagaatctcacaagccaaatggacctaaagaatcagtccgttatcagttggcaatctcacacctggccctaattcctgaaactcctctaagacaaagtgaatttccaaatggaaggctcactctccttgttactagactgtctggtggaaatatttatgtgttaaaggttcttgcctgccactctgaggaaatgtggtgtacagagagtcatcctgtcactgtggaaatgtttaacacaccagagaaaccttattccttggttccagagaacactagtttgcaatttaattggaaggctccattgaatgttaacctcatcagattttgggttgagctacagaagtggaaatacaatgagttttaccatgttaaaacttcatgcagccaaggtcctgcttatgtctgtaatatcacaaatctacaaccttatacttcatataatgtcagagtagtggtggtttataagacgggagaaaatagcacctcacttccagaaagctttaagacaaaagctggagtcccaaataaaccaggcattcccaaattactagaagggagtaaaaattcaatacagtgggagaaagctgaagataatggatgtagaattacatactatatccttgagataagaaagagcacttcaaataatttacagaaccagaatttaaggtggaagatgacatttaatggatcctgcagtagtgtttgcacatggaagtccaaaaacctgaaaggaatatttcagttcagagtagtagctgcaaataatctagggtttggtgaatatagtggaatcagtgagaatattatattagttggagatgatttttggataccagaaacaagtttcatacttactattatagttggaatatttctggttgttacaatcccactgacctttgtctggcatagaagattaaagaatcaaaaaagtgccaaggaaggggtgacagtgcttataaacgaagacaaagagttggctgagctgcgaggtctggcagccggagtaggcctggctaatgcctgctatgcaatacatactcttccaacccaagaggagattgaaaatcttcctgccttccctcgggaaaaactgactctgcgtctcttgctgggaagtggagcctttggagaagtgtatgaaggaacagcagtggacatcttaggagttggaagtggagaaatcaaagtagcagtgaagactttgaagaagggttccacagaccaggagaagattgaattcctgaaggaggcacatctgatgagcaaatttaatcatcccaacattctgaagcagcttggagtttgtctgctgaatgaaccccaatacattatcctggaactgatggagggaggagaccttcttacttatttgcgtaaagcccggatggcaacgttttatggtcctttactcaccttggttgaccttgtagacctgtgtgtagatatttcaaaaggctgtgtctacttggaacggatgcatttcattcacagggatctggcagctagaaattgccttgtttccgtgaaagactataccagtccacggatagtgaagattggagactttggactcgccagagacatctataaaaatgattactatagaaagagaggggaaggcctgctcccagttcggtggatggctccagaaagtttgatggatggaatcttcactactcaatctgatgtatggtcttttggaattctgatttgggagattttaactcttggtcatcagccttatccagctcattccaaccttgatgtgttaaactatgtgcaaacaggagggagactggagccaccaagaaattgtcctgatgatctgtggaatttaatgacccagtgctgggctcaagaacccgaccaaagacctacttttcatagaattcaggaccaacttcagttattcagaaattttttcttaaatagcatttataagtccagagatgaagcaaacaacagtggagtcataaatgaaagctttgaaggtgaagatggcgatgtgatttgtttgaattcagatgacattatgccagttgctttaatggaaacgaagaaccgagaagggttaaactatatggtacttgctacagaatgtggccaaggtgaagaaaagtctgagggtcctctaggctcccaggaatctgaatcttgtggtctgaggaaagaagagaaggaaccacatgcagacaaagatttctgccaagaaaaacaagtggcttactgcccttctggcaagcctgaaggcctgaactatgcctgtctcactcacagtggatatggagatgggtctgattaatagcgttgtttgggaaatagagagttgagataaacactctcattcagtagttactgaaagaaaactctgctagaatgataaatgtcatggtggtctataactccaaataaacaatgcaacgttcctgatttctaatcttggttctgagagccatttggtttcagttgtagcaatccccataccagctgcctgactttcagtagaattatgagatgaacactaagcatgtggaaagcttaggaagactcagaagtctggaagggaaacactgctctcccttctcccttgaggtgctttaggctcttacccacctttcagtttgggctgtaataaaaatatcttggccacatgtttagagacagaataggtgtgttcagcgatataaagaagaggctaaggagtaggctcaggggggtcaactgaactacagataatctcaaatgggaccaaggaaatgagaaataatttcacacatacagaagaaaccagcacctgtgacttgagaaatcacttggaaagctgttactgcaatgatatatatattatctttttttaattttttttttttttttttgagacgaagtcttgctctgttgcccaggctggagttcaatggcacgatctcggcactgcaaactccacctcctgggttcaagcaattctcgtgcctcagcctcctaagtagctgggattacaggcgtgtgccaccacgcccggctaatttttgtatttttagtagagatgaggtttcaccatattggccaggctggtctagaactcctgacctcgggatccacctgccttggcctcccaaagtgctggattacaggtgtgagccaccatgcctagccgatatatattgtctttaatcactactgtaaaatattttgtagttttgaggcttacaacagtagattcagtcatgttgaaaataagactgtgaagatcttttaagtcctgaagttttgcattctgtaatcttcagttgtataaaatcactctgacttgtgtgctattatggaaattaactagtataaagattgctatttgccatatctattttatgtataaaatacttaagattacattttgtatcaaattatgcttaaaattaaatataaatgattatacaatgttaa

SEQ ID NO: 12 - DNA, 2669 bp, Homo sapiens
  source 1..2669: mol_type = unassigned DNA
ggaggcctggcagctgcagctgggagcgcacagacggctgccccgcctgagcgaggcgggcgccgccgcgatgctgcgaggcggacggcgcgggcagcttggctggcacagctgggctgcggggccgggcagcctgctggcttggctgatactggcatctgcgggcgccgcaccctgccccgatgcctgctgcccccacggctcctcgggactgcgatgcacccgggatggggccctggatagcctccaccacctgcccggcgcagagaacctgactgagctctacatcgagaaccagcagcatctgcagcatctggagctccgtgatctgaggggcctgggggagctgagaaacctcaccatcgtgaagagtggtctccgtttcgtggcgccagatgccttccatttcactcctcggctcagtcgcctgaatctctccttcaacgctctggagtctctctcctggaaaactgtgcagggcctctccttacaggaactggtcctgtcggggaaccctctgcactgttcttgtgccctgcgctggctacagcgctgggaggaggagggactgggcggagtgcctgaacagaagctgcagtgtcatgggcaagggcccctggcccacatgcccaatgccagctgtggtgtgcccacgctgaaggtccaggtgcccaatgcctcggtggatgtgggggacgacgtgctgctgcggtgccaggtggaggggcggggcctggagcaggccggctggatcctcacagagctggagcagtcagccacggtgatgaaatctgggggtctgccatccctggggctgaccctggccaatgtcaccagtgacctcaacaggaagaacgtgacgtgctgggcagagaacgatgtgggccgggcagaggtctctgttcaggtcaacgtctccttcccggccagtgtgcagctgcacacggcggtggagatgcaccactggtgcatccccttctctgtggatgggcagccggcaccgtctctgcgctggctcttcaatggctccgtgctcaatgagaccagcttcatcttcactgagttcctggagccggcagccaatgagaccgtgcggcacgggtgtctgcgcctcaaccagcccacccacgtcaacaacggcaactacacgctgctggctgccaaccccttcggccaggcctccgcctccatcatggctgccttcatggacaaccctttcgagttcaaccccgaggaccccatccctgtctccttctcgccggtggacactaacagcacatctggagacccggtggagaagaaggacgaaacaccttttggggtctcggtggctgtgggcctggccgtctttgcctgcctcttcctttctacgctgctccttgtgctcaacaaatgtggacggagaaacaagtttgggatcaaccgcccggctgtgctggctccagaggatgggctggccatgtccctgcatttcatgacattgggtggcagctccctgtcccccaccgagggcaaaggctctgggctccaaggccacatcatcgagaacccacaatacttcagtgatgcctgtgttcaccacatcaagcgccgggacatcgtgctcaagtgggagctgggggagggcgcctttgggaaggtcttccttgctgagtgccacaacctcctgcctgagcaggacaagatgctggtggctgtcaaggcactgaaggaggcgtccgagagtgctcggcaggacttccagcgtgaggctgagctgctcaccatgctgcagcaccagcacatcgtgcgcttcttcggcgtctgcaccgagggccgccccctgctcatggtctttgagtatatgcggcacggggacctcaaccgcttcctccgatcccatggacctgatgccaagctgctggctggtggggaggatgtggctccaggccccctgggtctggggcagctgctggccgtggctagccaggtcgctgcggggatggtgtacctggcgggtctgcattttgtgcaccgggacctggccacacgcaactgtctagtgggccagggactggtggtcaagattggtgattttggcatgagcagggatatctacagcaccgactattaccgtgtgggaggccgcaccatgctgcccattcgctggatgccgcccgagagcatcctgtaccgtaagttcaccaccgagagcgacgtgtggagcttcggcgtggtgctctgggagatcttcacctacggcaagcagccctggtaccagctctccaacacggaggcaatcgactgcatcacgcagggacgtgagttggagcggccacgtgcctgcccaccagaggtctacgccatcatgcggggctgctggcagcgggagccccagcaacgccacagcatcaaggatgtgcacgcccggctgcaagccctggcccaggcacctcctgtctacctggatgtcctgggctagggggccggcccaggggctgggagtggttagccggaatactggggcctgccctcagcatcccccatagctcccagcagccccagggtgatctcaaagtatctaattcaccctcagcatgtgggaagggacaggtgggggctgggagtagaggatgttcctgcttctctaggcaaggtcccgtcatagcaattatatttattatcccttg